(19) United States
(12) Patent Application Publication
(10) Pub. No.: US 2025/0287832 A1
(43) Pub. Date: Sep. 11, 2025

(54) ORGANIC ELECTROLUMINESCENT COMPOUND, A PLURALITY OF HOST MATERIALS AND ORGANIC ELECTROLUMINESCENT COMPRISING THE SAME

(71) Applicant: DuPont Specialty Materials Korea Ltd., Chungcheongnam-do (KR)

(72) Inventors: Yoo-Jin DOH, Gyeonggi-do (KR); Chi-Sik KIM, Gyeonggi-do (KR); HaeYeon KIM, Gyeonggi-do (KR); Mi-Ja LEE, Gyeonggi-do (KR); Young-Kwang KIM, Gyeonggi-do (KR)

(21) Appl. No.: 19/068,416

(22) Filed: Mar. 3, 2025

(30) Foreign Application Priority Data
Mar. 11, 2024 (KR) 10-2024-0034124
Jan. 13, 2025 (KR) 10-2025-0004906

(51) Int. Cl. H10K 85/60; C07C 15/28; C07C 15/30; C07D 307/77; C07D 405/10; C07D 409/10; C09K 11/02; C09K 11/06; H10K 50/11; H10K 101/00

(52) U.S. Cl. CPC H10K 85/626; C07C 15/28; C07C 15/30; C07D 307/77; C07D 405/10; C07D 409/10; C09K 11/02; C09K 11/06; H10K 85/615; H10K 85/6574; C09K 2211/1007; C09K 2211/1011; H10K 50/11; H10K 2101/90

(57) ABSTRACT

The present disclosure relates to a plurality of host materials comprising at least one first host compound represented by Formula 1 and at least one second host compound represented by Formula 2, wherein the first host compound and the second compound are different from each other; an organic electroluminescent compound represented by Formula 11; and an organic electroluminescent device comprising the same. It is possible to produce an organic electroluminescent device having improved driving voltage, efficiency, and/or lifetime properties, by comprising the organic electroluminescent compound according to the present disclosure or a specific combination of compounds according to the present disclosure as host materials.

### TECHNICAL FIELD

The present disclosure relates to an organic electroluminescent compound, a plurality of host materials, and an organic electroluminescent device comprising the same.

### BACKGROUND ART

In 1987, Tang et al. of Eastman Kodak first developed a small-molecule green organic electroluminescent device (OLED) of TPD/Alq₃ bilayer consisting of a light-emitting layer and a charge transport layer. Since then, research on OLEDs has been rapidly carried out, and OLEDs have been commercialized. At present, phosphorescent materials, which provide excellent luminous efficiency in realizing panels, are mainly used in OLEDs. In various applications such as TVs and lighting, the lifetime of OLEDs is often insufficient, and higher OLED efficiency is still required. Generally, the lifetime of an OLED decreases as its luminance increases. Thus, OLEDs with high luminous efficiency and/or long lifetime are essential for long-term use and high-resolution displays.

In order to improve luminous efficiency, driving voltage, and/or lifetime, various materials or concepts for an organic layer of an organic electroluminescent device have been proposed, but these have not proved to be satisfactory in practical use. In addition, there is a continuous demand for the development of an organic electroluminescent material with enhanced performance, such as improved driving voltage, luminous efficiency, power efficiency, and/or lifetime properties, as compared to a previously disclosed combination of specific compounds.

Meanwhile, Korean Patent Application Laid-Open No. 2006-0108642 (published on Oct. 18, 2006) discloses asymmetric monoanthracene derivaties, Korean Patent Application Laid-Open No. 2015-0141271 (published on Dec. 18, 2015) discloses an organic electroluminescent device comprising a combination of anthracene derivatives, and Korean Patent Application Laid-Open No. 2020-0034649 (published on Mar. 31, 2020) discloses a compound containing an anthracene structure as a host material. However, the aforementioned references do not specifically disclose a specific compound or the host materials comprising a specific combination of compounds according to the present disclosure. In addition, there is a continuing need to develop light-emitting materials with improved performance, such as improved driving voltage, luminous efficiency, power efficiency, and/or lifetime properties, compared to previously disclosed compounds.

### DISCLOSURE OF INVENTION

### Technical Problem

The objective of the present disclosure is to provide an organic electroluminescent compound having a novel structure suitable for applying to an organic electroluminescent device. Another objective of the present disclosure is to provide a plurality of host materials capable of producing an organic electroluminescent device having low driving voltage, high efficiency, and/or improved lifetime properties. Another objective of the present disclosure is to provide an organic electroluminescent device having low driving voltage, high efficiency, and/or improved lifetime properties by comprising a compound according to the present disclosure as a single host material or a specific combination of compounds according to the present disclosure as a plurality of host materials.

### Solution to Problem

As a result of intensive studies conducted to solve the above technical problems, the present inventors have found that the above objective can be achieved through a plurality of host materials comprising at least one first host compound represented by the following Formula 1 and at least one second host compound represented by the following Formula 2, wherein the first host compound and the second compound are different from each other; or an organic electroluminescent compound represented by the following Formula 11; and a organic electroluminescent device comprising the same.

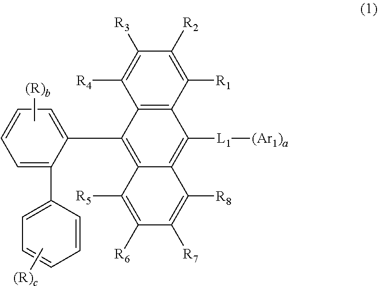

In Formula 1,
L₁ represents a single bond, a phenylene unsubstituted or substituted with deuterium, a naphthylene unsubstituted or substituted with deuterium, or a phenanthrenylene unsubstituted or substituted with deuterium;
Ar₁ each independently represents a substituted or unsubstituted (C₆-C₃₀)aryl, or a substituted or unsubstituted (3- to 30-membered)heteroaryl;
R and R₁ to R₈ each independently represent hydrogen or deuterium; and
a represents an integer of 1 or 2, b represents an integer of 4, and c represents an integer of 5; and each Ar₁ may be the same as or different from each other;
with a proviso that Formula 1 is not represented as

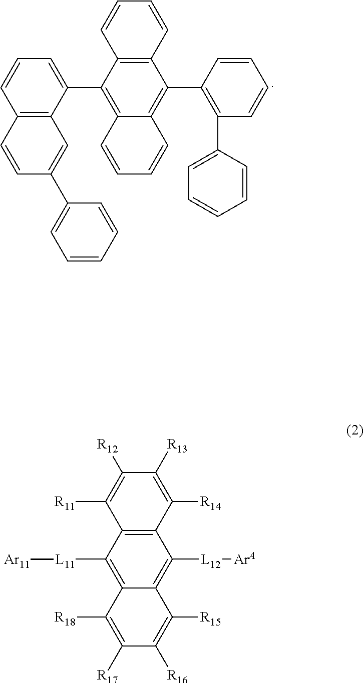

In Formula 2,
Ar^{A} represents a substituted or unsubstituted (C₆-C₃₀)aryl, a substituted or unsubstituted (13- to 30-membered)heteroaryl, or the following Formula A-1:

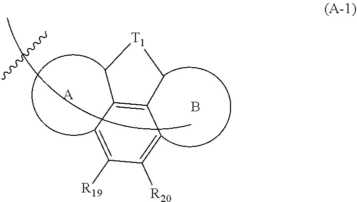

wherein T₁ represents O, S, or CRₐR_{b};
Ring A and Ring B each independently represent a substituted or unsubstituted (C₆-C₃₀)arene, or a substituted or unsubstituted (3- to 30-membered) heteroarene;
Ar₁₁ represents a substituted or unsubstituted (C₆-C₃₀)aryl, or a substituted or unsubstituted (13- to 30-membered)heteroaryl;
R₁₁ to R₁₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄);
R₁₉ and R₂₀ each independently represent the position linked to L₁₂, or represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered) heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄);
Rₐ and R_{b} each independently represent a substituted or unsubstituted (C₁-C₃₀)alkyl, or a substituted or unsubstituted (C₆-C₃₀)aryl, or may be linked to each other to form a ring(s);
L₁₁ to L₁₃ each independently represent a single bond, a substituted or unsubstituted (C₆-C₃₀)arylene, or a substituted or unsubstituted (3- to 30-membered) heteroarylene; and
Ar₁₃ and Ar₁₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring group of a (C₃-C₃₀) aliphatic ring(s) and a (C₆-C₃₀) aromatic ring(s).

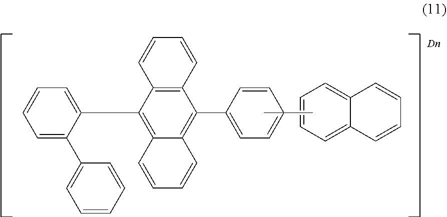

In Formula 11, Dₙ signifies that n hydrogens are replaced with deuterium, and n is an integer from 1 to 28.

### Advantageous Effects of Invention

The organic electroluminescent compound according to the present disclosure exhibits performance suitable for use in an organic electroluminescent device. In addition, by comprising the compound according to the present disclosure as a single host material, or a specific combination of compounds according to the present disclosure as a plurality of host materials, an organic electroluminescent device can be produced which has lower driving voltage, higher efficiency, and/or improved lifetime properties compared to a conventional organic electroluminescent device, and a display device or lighting device using the same can be produced.

### MODE FOR THE INVENTION

Hereinafter, the present disclosure will be described in detail. However, the following description is intended to explain the present disclosure and is not meant in any way to restrict the scope of the present disclosure.

The present disclosure relates to a plurality of host materials comprising at least one first host compound represented by Formula 1 and at least one second host compound represented by Formula 2, wherein the first host compound and the second compound are different from each other; and an organic electroluminescent device comprising the host materials. In addition, the present disclosure relates to an organic electroluminescent compound represented by Formula 11, and an organic electroluminescent device comprising the same. Furthermore, the present disclosure relates to an organic electroluminescent device comprising a compound represented by Formula 1 as a host material(s).

The “organic electroluminescent compound” in the present disclosure is a compound that may be used in an organic electroluminescent device, and may be comprised in any material layer constituting an organic electroluminescent device, as necessary.

The “an organic electroluminescent material” in the present disclosure is a material that may be used in an organic electroluminescent device, and may comprise at least one compound. If necessary, the organic electroluminescent material may be comprised in any layer constituting an organic electroluminescent device. For example, the organic electroluminescent material may be a hole injection material, a hole transport material, a hole auxiliary material, a light-emitting auxiliary material, an electron-blocking material, a light-emitting material (including a host material and a dopant material), an electron buffer material, a hole-blocking material, an electron transport material, an electron injection material, etc.

The “plurality of organic electroluminescent materials” in the present disclosure is an organic electroluminescent material that is a combination of at least two compounds, which may be comprised in any layer constituting an organic electroluminescent device. It may be both a material before being comprised in an organic electroluminescent device (e.g., before vapor deposition) and a material after being comprised in an organic electroluminescent device (e.g., after vapor deposition). For example, the plurality of organic electroluminescent materials may be a combination of at least two compounds, which may be comprised in at least one layer(s) of a hole injection layer, a hole transport layer, a hole auxiliary layer, a light-emitting auxiliary layer, an electron-blocking layer, a light-emitting layer, an electron buffer layer, a hole-blocking layer, an electron transport layer, and an electron injection layer. At least two of these compounds may be comprised in the same layer or different layers, and may be mixture-deposited, co-deposited, or separately deposited.

The “plurality of host materials” in the present disclosure is an organic electroluminescent material in which two or more host materials are combined. It may be both a material before being comprised in an organic electroluminescent device (e.g., before vapor deposition) and a material after being comprised in an organic electroluminescent device (e.g., after vapor deposition). The plurality of host materials of the present disclosure may be comprised in any light-emitting layer constituting an organic electroluminescent device. At least two compounds comprised in the plurality of host materials may be comprised together in one light-emitting layer or may each be comprised in different light-emitting layers. When two or more host materials are comprised in one layer, for example, they may be mixture-evaporated to form a layer, or they may be separately and simultaneously co-evaporated to form a layer.

Herein, the “(C₁-C₃₀)alkyl” is meant to be a linear or branched alkyl having 1 to 30 carbon atoms constituting a chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the “(C₃-C₃₀)cycloalkyl” is meant to be a mono- or polycyclic hydrocarbon having 3 to 30 ring backbone carbon atoms, in which the number of ring backbone carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc.

Herein, the “(C₆-C₃₀)aryl”, “(C₆-C₃₀)arylene”, or “(C₆-C₃₀)arene” is meant to be a monocyclic or fused ring-type radical derived from an aromatic hydrocarbon having 6 to 30 ring backbone carbon atoms that can be partially saturated, in which the number of ring backbone carbon atoms is preferably 6 to 20, and more preferably 6 to 15. The above aryl may comprise a spiro structure. The above aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzoanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluoren]yl, spiro[fluorene-benzofluoren]yl, azulenyl, tetramethyl-dihydrophenanthrenyl, etc. Specifically, the above aryl may include o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-11,11-diphenyl-5-benzo[a]fluorenyl, 11,11-diphenyl-6-benzo[a]fluorenyl, benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-11,11-diphenyl-4-benzo[b]fluorenyl, 11,11-diphenyl-3-benzo[b]fluorenyl, benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the “(3- to 30-membered)heteroaryl”, “(3- to 30-membered) heteroarylene”, or “(3- to 30-membered) heteroarene” is meant to be an aryl or arylene group having 3 to 30 ring backbone atoms, and comprising at least one heteroatom selected from the group consisting of B, N, O, S, Si, and P, in which the number of ring backbone atoms is preferably 3 to 30, and more preferably 5 to 20. The number of the heteroatoms is preferably 1 to 4. The above heteroaryl or heteroarylene may be a monocyclic ring, or a fused ring condensed with at least one benzene ring, and may be partially saturated. In addition, the above heteroaryl or heteroarylene may be one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the above heteroaryl may include 1-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl) pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-4-naphtho-[2,1-b]-benzothiophenyl, 3-naphtho-[2,1-b]-benzothiophenyl, benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc.

Herein, “a fused ring group of a (C₃-C₃₀) aliphatic ring(s) and a (C₆-C₃₀) aromatic ring(s)” is meant to be a functional group of a ring in which at least one aliphatic ring having 3 to 30 ring backbone carbon atoms, preferably 3 to 25 ring backbone carbon atoms, more preferably 3 to 18 ring backbone carbon atoms, is fused with at least one aromatic ring having 6 to 30 ring backbone carbon atoms, preferably 6 to 25 ring backbone carbon atoms, more preferably 6 to 18 ring backbone carbon atoms. Specific examples of the fused ring group include a fused ring group of one or more benzene and one or more cyclohexane, or a fused ring group of one or more naphthalene and one or more cyclopentane, etc. Herein, the carbon atom of the fused ring group of a (C₃-C₃₀) aliphatic ring(s) and a (C₆-C₃₀) aromatic ring(s) may be replaced with one or more heteroatoms selected from B, N, O, S, Si, and P, preferably one or more heteroatoms selected from N, O, and S. Herein, “halogen” includes F, Cl, Br, and I.

In addition, “ortho-” (“o-”), “meta-” (“m-”), and “para-” (“p-”) are prefixes which represent the relative positions of substituents respectively. The prefix “ortho-” indicates that two substituents are adjacent to each other; for example, when two substituents in a benzene derivative occupy positions 1 and 2, this is called an “ortho-” configuration. The prefix “meta-” indicates that two substituents are at positions 1 and 3; for example, when two substituents in a benzene derivative occupy positions 1 and 3, this is called a “meta-” configuration. The prefix “para-” indicates that two substituents are at positions 1 and 4; for example, when two substituents in a benzene derivative occupy positions 1 and 4, this is called a “para-” configuration.

Herein, “a ring formed by being linked to an adjacent substituent” means that at least two adjacent substituents are linked to or fused with each other to form a substituted or unsubstituted, mono- or polycyclic, (3- to 30-membered) alicyclic or aromatic ring, or a combination thereof. Preferably, the ring may be a substituted or unsubstituted, mono- or polycyclic, (5- to 25-membered) alicyclic or aromatic ring, or a combination thereof. In addition, the ring may contain at least one heteroatom selected from B, N, O, S, Si, and P, preferably at least one heteroatom selected from N, O, and S. According to one embodiment of the present disclosure, the number of the ring backbone atoms may be 5- to 20-membered, and according to another embodiment of the present disclosure, the number of the ring backbone atoms may be 5- to 15-membered. For example, the ring may be a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

Herein, “substituted” in the expression “substituted or unsubstituted” means that a hydrogen atom in a certain functional group is replaced with another atom or another functional group, i.e., a substituent. The substituent also includes those in which two or more substituents are linked. For example, the substituent formed by linking two or more substituents may be pyridine-triazine. That is, pyridine-triazine may be interpreted as a heteroaryl, or a substituent(s) with two heteroaryls linked. In the formulas of the present disclosure, the substituent(s) of the substituted alkyl, the substituted alkenyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted arene, and the substituted heteroarene each independently are substituted with at least one selected from the group consisting of deuterium, a halogen, a cyano, carboxyl, nitro, hydroxyl, phosphine oxide, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀) arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, amino, mono- or di(C₁-C₃₀)alkylamino, mono- or di(C₂-C₃₀)alkenylamino, mono- or di(C₆-C₃₀)arylamino, mono- or di(3- to 30-membered)heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, (C₆-C₃₀)arylphosphinyl, di(C₁-C₃₀)alkylboronyl, (C₁-C₃₀)alkyl(C₆-C₃₀)arylboronyl, (C₆-C₃₀)aryl(C₁-C₃₀)alkyl, (C₁-C₃₀)alkyl(C₆-C₃₀)aryl, and a combination thereof, which may be further substituted with deuterium. According to one embodiment of the present disclosure, the substituent(s) each independently may be at least one selected from the group consisting of deuterium, (C₁-C₂₀)alkyl unsubstituted or substituted with deuterium, and (C₆-C₂₅)aryl unsubstituted or substituted with deuterium. According to another embodiment of the present disclosure, the substituent(s) each independently may be at least one selected from the group consisting of deuterium, (C₁-C₁₀)alkyl unsubstituted or substituted with deuterium, and (C₆-C₁₈)aryl unsubstituted or substituted with deuterium. For example, the substituent(s) each independently may be at least one selected from the group consisting of deuterium, methyl, phenyl, naphthyl, biphenyl, and phenanthrenyl, which may be substituted with deuterium.

Herein, if a substituent is not indicated in the formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In cases where a substituent is not indicated in the formula or compound structure in the present disclosure, if the substituent is not explicitly excluded, such as 0% deuterium, 100% hydrogen, and all substituents being hydrogen, hydrogen and deuterium may be used together in a compound. Deuterium is one of the isotopes of hydrogen and an element with a deuteron, consisting of one proton and one neutron, as its nucleus. It can be represented as hydrogen-2, whose element symbol can also be written as D or ²H. Isotopes are atoms with the same atomic number (Z) but different mass numbers (A), and can also be interpreted as elements with the same number of protons but different numbers of neutrons.

Herein, “a combination thereof” refers to a combination of one or more elements from the corresponding list to form a known or chemically stable arrangement that can be envisioned by one skilled in the art from the corresponding list. For example, alkyl and deuterium can be combined to form a partially or fully deuterated alkyl group; halogen and alkyl can be combined to form a halogenated alkyl substituent; and halogen, alkyl, and aryl can be combined to form a halogenated arylalkyl. For example, a preferred combination of substituents includes up to 50 atoms that are not hydrogen or deuterium, up to 40 atoms that are not hydrogen or deuterium, or up to 30 atoms that are not hydrogen or deuterium, but in many cases, a preferred combination of substituents may comprise up to 20 atoms that are not hydrogen or deuterium.

In the formulas of the present disclosure, when there are multiple substituents represented by the same symbol, each substituent represented by the same symbol may be the same as or different from each other.

According to one embodiment of the present disclosure, at least one of Formula 1 and Formula 2 may contain deuterium.

In Formula 1, L₁ represents a single bond, a phenylene unsubstituted or substituted with deuterium, a naphthylene unsubstituted or substituted with deuterium, a biphenylene unsubstituted or substituted with deuterium, or a phenanthrenylene unsubstituted or substituted with deuterium.

In Formula 1, Ar₁ each independently represents a substituted or unsubstituted (C₆-C₃₀) aryl, or a substituted or unsubstituted (3- to 30-membered)heteroaryl. According to one embodiment of the present disclosure, Ar₁ each independently represents a substituted or unsubstituted (C₆-C₂₅)aryl. According to another embodiment of the present disclosure, Ar₁ each independently represents a (C₆-C₁₈)aryl unsubstituted or substituted with deuterium. For example, Ar₁ each independently may be a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof.

In Formula 1, R and R₁ to R₈ each independently represent hydrogen or deuterium.

In Formula 1, a represents an integer of 1 or 2, b represents an integer of 4, and c represents an integer of 5; each Ar₁ may be the same as or different from each other.

In Formula 2, Ar₁₁ represents a substituted or unsubstituted (C₆-C₃₀)aryl, or a substituted or unsubstituted (13- to 30-membered)heteroaryl. According to one embodiment of the present disclosure, Ar₁₁ represents a substituted or unsubstituted (C₆-C₂₅)aryl, or a substituted or unsubstituted (13- to 25-membered)heteroaryl. According to another embodiment of the present disclosure, Ar₁₁ represents a substituted or unsubstituted (C₆-C₂₅)aryl, or a substituted or unsubstituted (13- to 17-membered)heteroaryl. Their substituents may be at least one selected from the group consisting of deuterium, (C₁-C₁₀)alkyl, (C₆-C₃₀)aryl, and a combination thereof. For example, Ar₁₁ may be a phenyl, a phenyl substituted with a phenanthrenyl(s), a naphthyl, a naphthylphenyl, a phenylnaphthyl, a biphenyl, a phenanthrenyl, a terphenyl, a triphenylenyl, a dimethylfluorenyl, a diphenylfluorenyl, a dimethylbenzofluorenyl, a spirobifluorenyl, a carbazolyl unsubstituted or substituted with a phenyl(s), a benzocarbazolyl, a dibenzofuranyl unsubstituted or substituted with a phenyl(s), a dibenzothiophenyl unsubstituted or substituted with a phenyl(s), a benzonaphthofuranyl, or a benzonaphthothiophenyl, etc., which may be further substituted with deuterium.

In Formula 2, R₁₁ to R₁₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄). For example, R₁₁ to R₁₈ each independently may represent hydrogen or deuterium.

In Formula 2, Ar^{A} represents a substituted or unsubstituted (C₆-C₃₀)aryl, a substituted or unsubstituted (13- to 30-membered)heteroaryl, or the above Formula A-1. According to one embodiment of the present disclosure, Ar^{A} represents a substituted or unsubstituted (C₆-C₂₅)aryl, a substituted or unsubstituted (13- to 25-membered)heteroaryl, or the above Formula A-1. According to another embodiment of the present disclosure, Ar^{A} represents a (C₆-C₂₀)aryl unsubstituted or substituted with deuterium or a (C₆-C₃₀)aryl(s), or the above Formula A-1. For example, Ar^{A} may be Formula A-1, or a phenyl, a phenyl substituted with a phenanthrenyl(s), a naphthyl, a phenylnaphthyl, a naphthylphenyl, a biphenyl, a phenanthrenyl, or a terphenyl, etc., which may be further substituted with deuterium.

In Formula A-1, T₁ represents O, S, or CRₐR_{b}.

In Formula A-1, Ring A and Ring B each independently represent a substituted or unsubstituted (C₆-C₃₀)arene, or a substituted or unsubstituted (3- to 30-membered) heteroarene. According to one embodiment of the present disclosure, Ring A and Ring B each independently represent a substituted or unsubstituted (C₆-C₂₅)arene. According to another embodiment of the present disclosure, Ring A and Ring B each independently represent (C₆-C₁₈)arene unsubstituted or substituted with deuterium. For example, Ring A and Ring B each independently may be a substituted or unsubstituted benzene or naphthalene ring, and its substituents may be at least one selected from the group consisting of deuterium, phenyl, naphthyl, biphenyl, and a combination thereof.

In Formula A-1, R₁₉ and R₂₀ each independently represent the position linked to L₁₂, or represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄). According to one embodiment of the present disclosure, R₁₉ and R₂₀ each independently represent the position linked to L₁₂, or represent hydrogen, deuterium, or a substituted or unsubstituted (C₆-C₂₅)aryl. According to another embodiment of the present disclosure, R₁₉ and R₂₀ each independently represent the position linked to L₁₂, or represent hydrogen, deuterium, or a (C₆-C₁₈)aryl unsubstituted or substituted with deuterium. For example, R₁₉ and R₂₀ each independently represent the position linked to L₁₂, or represent hydrogen, deuterium, a phenyl, a naphthyl, or a biphenyl, etc., which may be further substituted with deuterium.

Rₐ and R_{b} each independently represent a substituted or unsubstituted (C₁-C₃₀)alkyl, or a substituted or unsubstituted (C₆-C₃₀)aryl, or may be linked to each other to form a ring(s). According to one embodiment of the present disclosure, Rₐ and R_{b} each independently represent a substituted or unsubstituted (C₁-C₂₀)alkyl. According to another embodiment of the present disclosure, Rₐ and R_{b} each independently represent a substituted or unsubstituted (C₁-C₁₀)alkyl. For example, Rₐ and R_{b} each independently may be a methyl unsubstituted or substituted with deuterium.

L₁₁ to L₁₃ each independently represent a single bond, a substituted or unsubstituted (C₆-C₃₀)arylene, or a substituted or unsubstituted (3- to 30-membered) heteroarylene. According to one embodiment of the present disclosure, L₁₁ to L₁₃ each independently represent a single bond, a substituted or unsubstituted (C₆-C₂₅)arylene, or a substituted or unsubstituted (5- to 25-membered)heteroarylene. According to another embodiment of the present disclosure, L₁₁ to L₁₃ each independently represent a single bond; a (C₆-C₁₈)arylene unsubstituted or substituted with deuterium, a (C₁-C₃₀)alkyl(s) and/or a (C₆-C₃₀)aryl(s); or a (5- to 20-membered)heteroarylene unsubstituted or substituted with deuterium. For example, L₁₁ to L₁₃ each independently represent a single bond, a phenylene unsubstituted or substituted with a methyl(s) or a phenyl(s), a naphthylene unsubstituted or substituted with a phenyl(s), a biphenylene, a phenanthrenylene, a carbazolylene, a dibenzofuranylene, or a dibenzothiophenylene, etc., which may be further substituted with deuterium.

Ar₁₃ and Ar₁₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring group of a (C₃-C₃₀) aliphatic ring(s) and a (C₆-C₃₀) aromatic ring(s).

According to one embodiment of the present disclosure, Ar₁₁ and Ar^{A} each independently may be selected from a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof. According to another embodiment of the present disclosure, Ar₁₁ may be selected from a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof; and Ar^{A} may represent a dibenzofuranyl unsubstituted or substituted with deuterium, or a dibenzothiophenyl unsubstituted or substituted with deuterium.

According to one embodiment of the present disclosure, Ar^{A} is represented by the following Formula B-1:

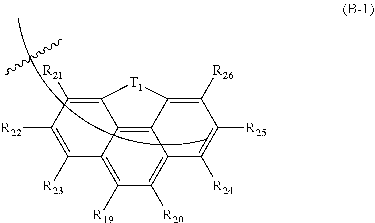

In Formula B-1, R₂₁ to R₂₆ each independently represent the position linked to L₁₂, or represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s). According to one embodiment of the present disclosure, R₂₁ to R₂₆ each independently represent the position linked to L₁₂, or represent hydrogen, deuterium, or a substituted or unsubstituted (C₆-C₂₅)aryl, or may be linked to the adjacent substituent to form a substituted or unsubstituted, mono- or polycyclic, (3- to 30-membered) alicyclic or aromatic ring, or a combination thereof. According to another embodiment of the present disclosure, R₂₁ to R₂₆ each independently represent the position linked to L₁₂, or represent hydrogen, deuterium, or a substituted or unsubstituted (C₆-C₁₈)aryl, or may be linked to the adjacent substituent to form a substituted or unsubstituted, mono- or polycyclic, (3- to 20-membered) aromatic ring. For example, R₂₁ to R₂₆ each independently represent the position linked to L₁₂, or represent hydrogen, deuterium, or a (C₆-C₁₈)aryl unsubstituted or substituted with deuterium, or may be linked to the adjacent substituent to form a benzene ring unsubstituted or substituted with deuterium.

In Formula B-1, T₁, R₁₉, R₂₀, L₁₃, Ar₁₃, and Ar₁₄ are as defined in Formula 2 above.

Formula 2 may be represented by the following Formula 2-1 or 2-2.

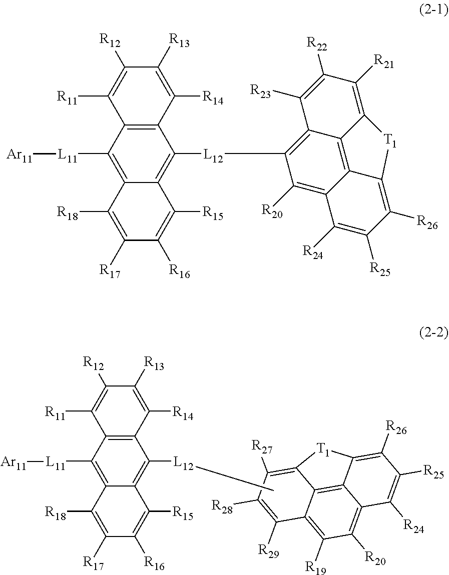

In Formulas 2-1 and 2-2,
R₂₁ to R₂₆ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s);
R₂₇ to R₂₉ each independently represent the position linked to L₁₂, or represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s); and
T₁, R₁₁ to R₂₀, L₁₁ to L₁₃, Ar₁₁, Ar₁₃, and Ar₁₄ are as defined in Formula 2 above.

The compound represented by Formula 1 may be more specifically at least one selected from the group consisting of the following compounds, but is not limited thereto.

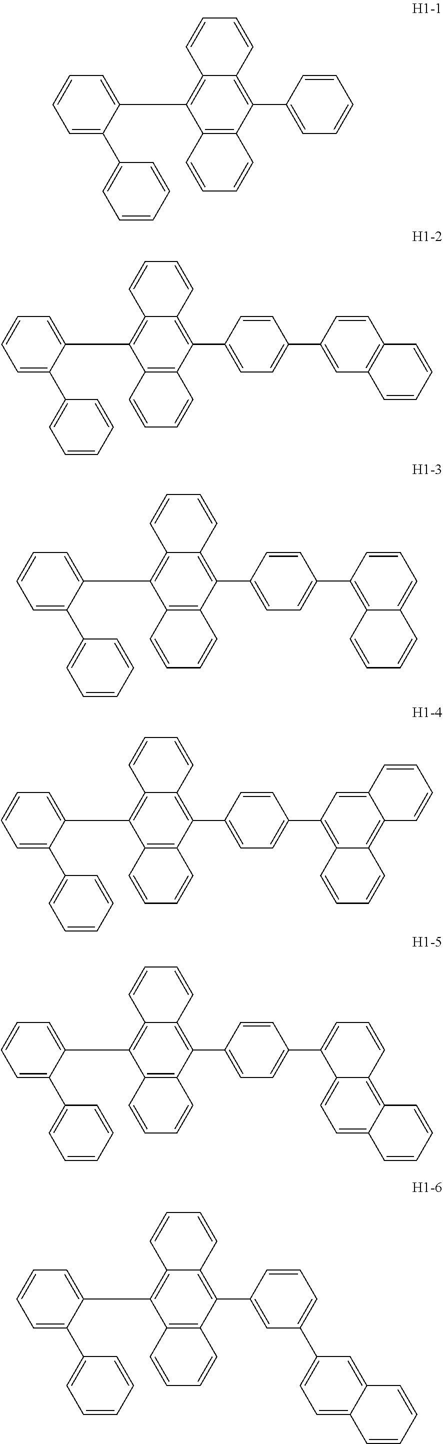

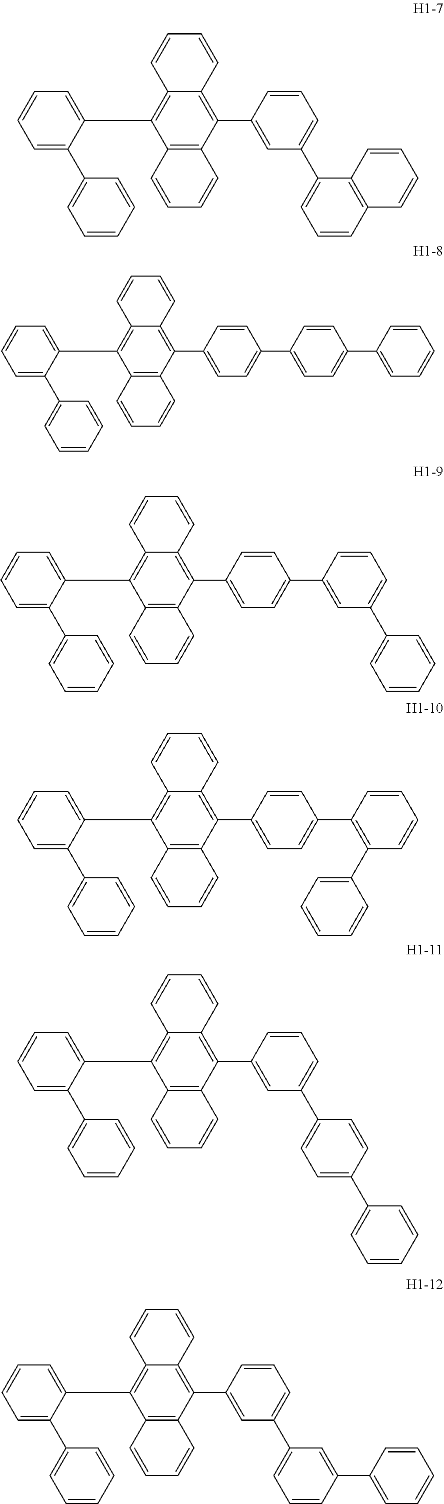

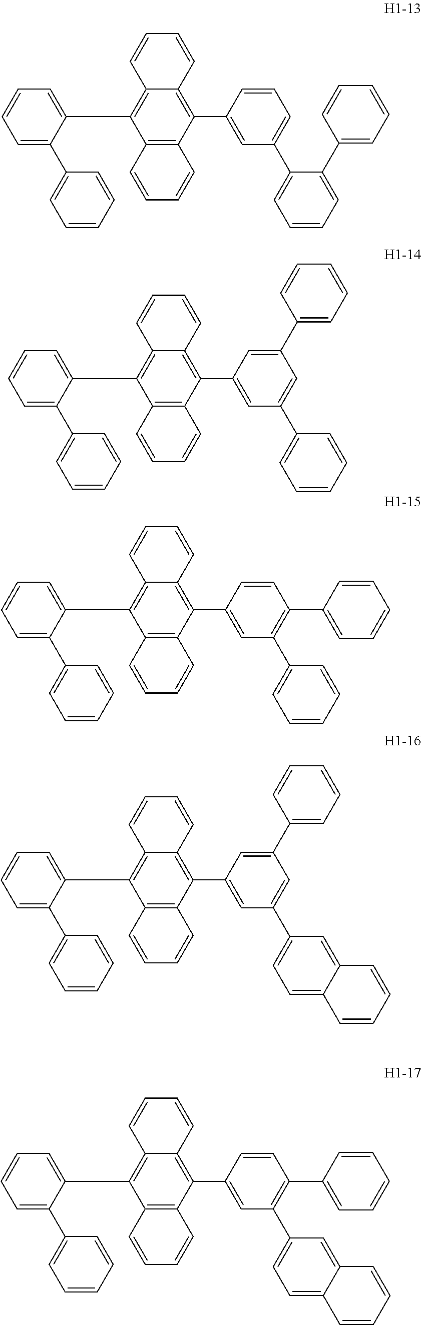

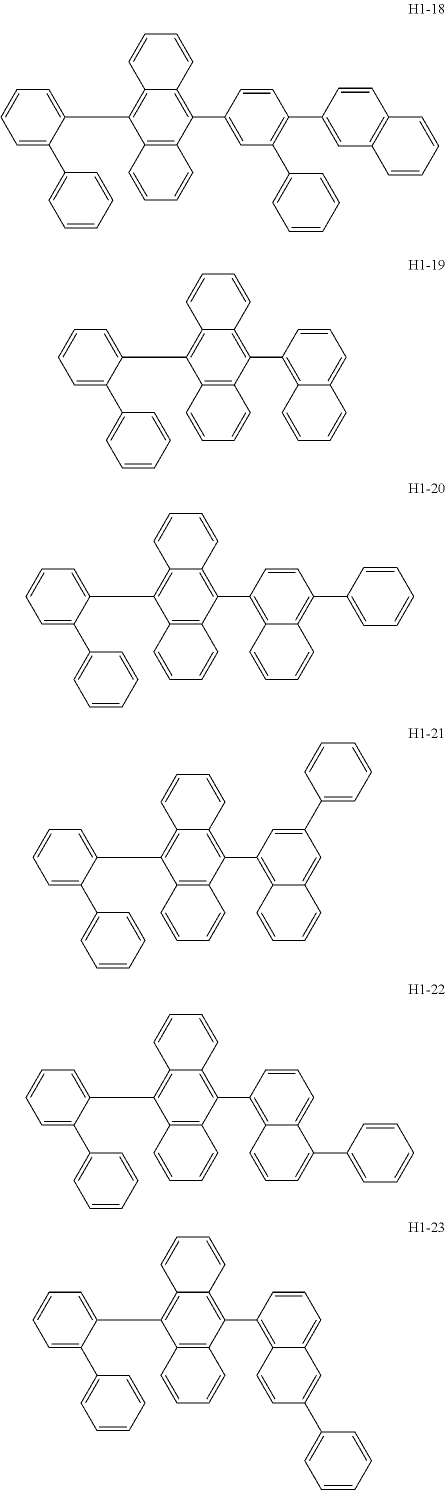

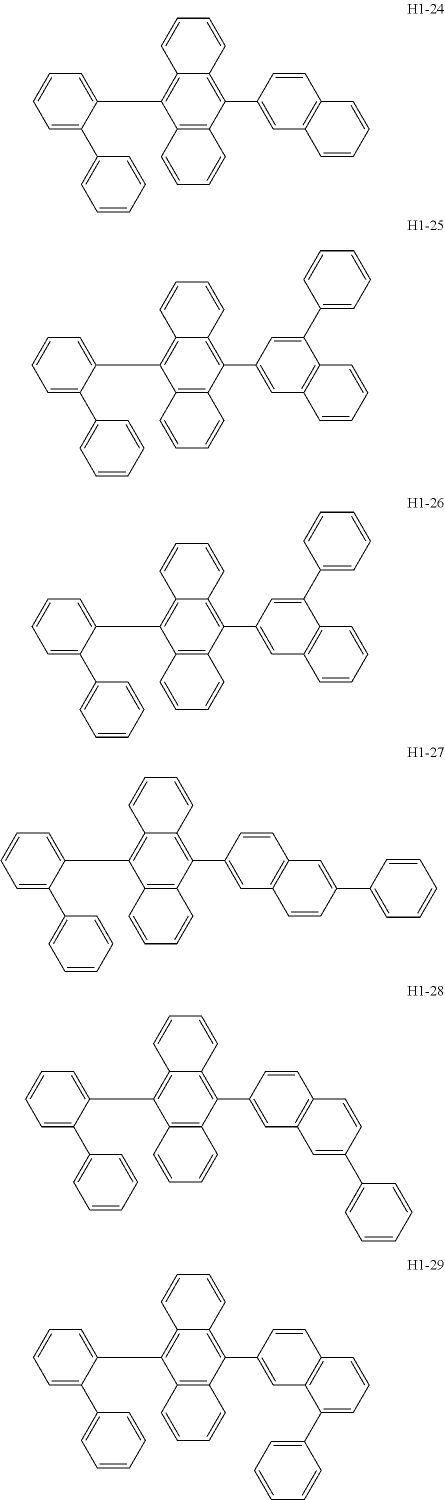

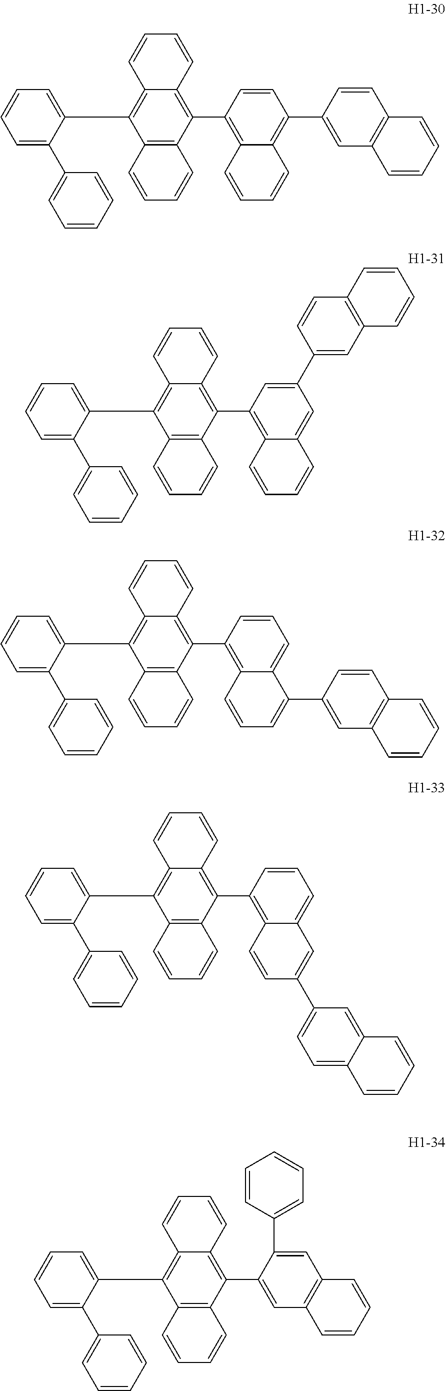

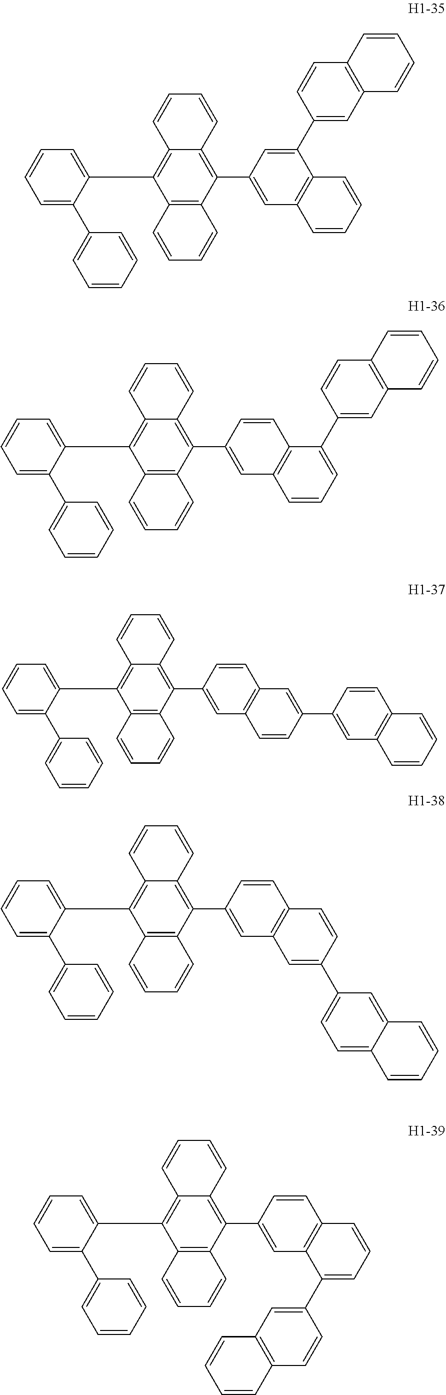

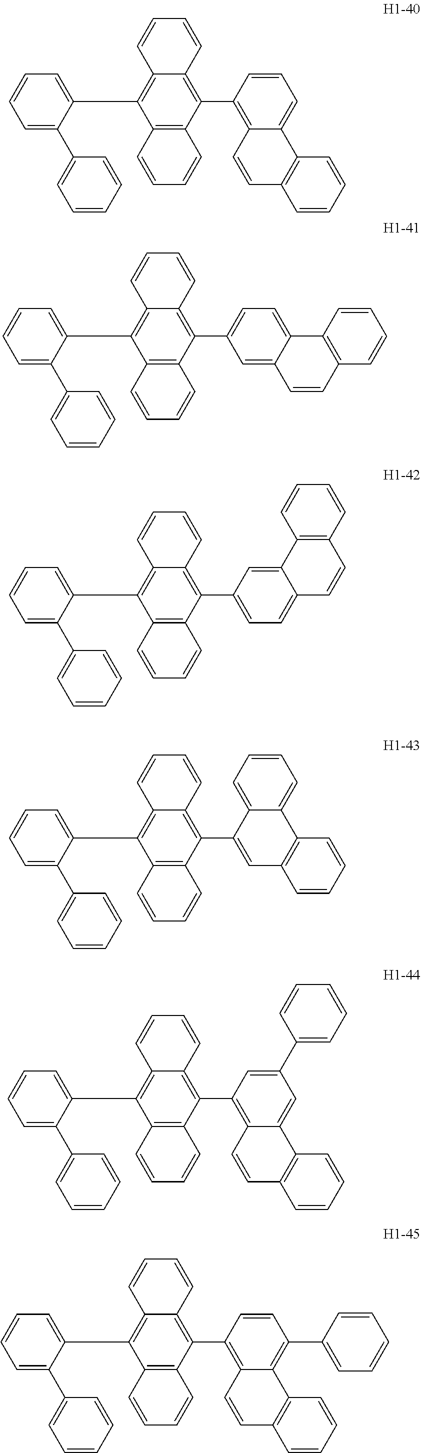

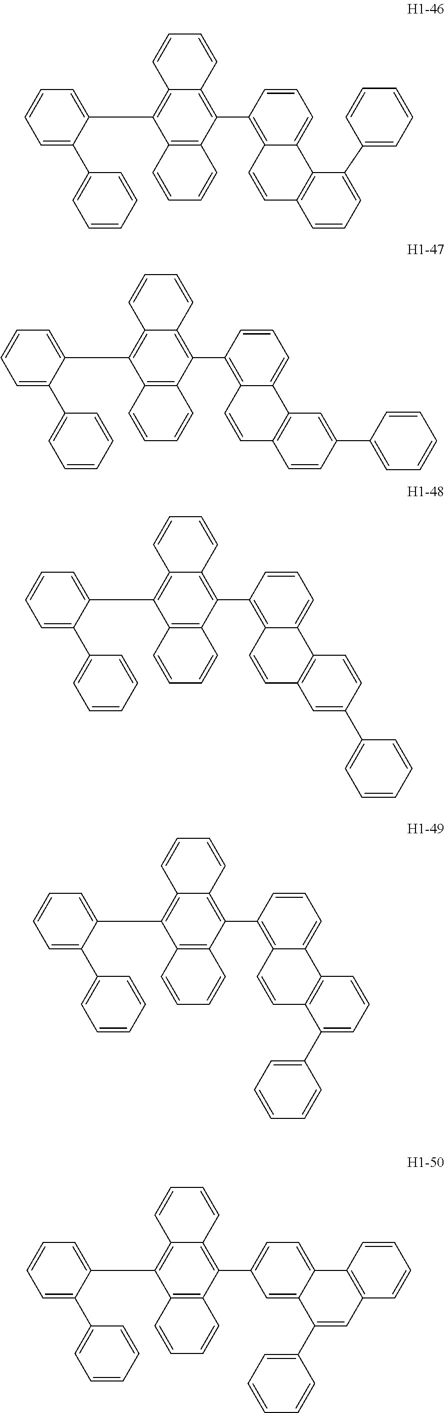

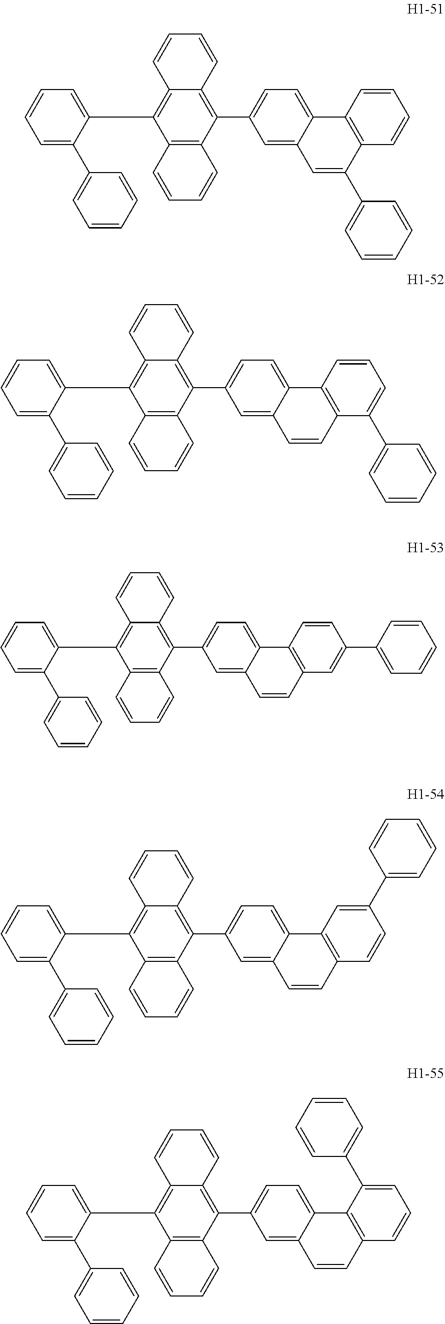

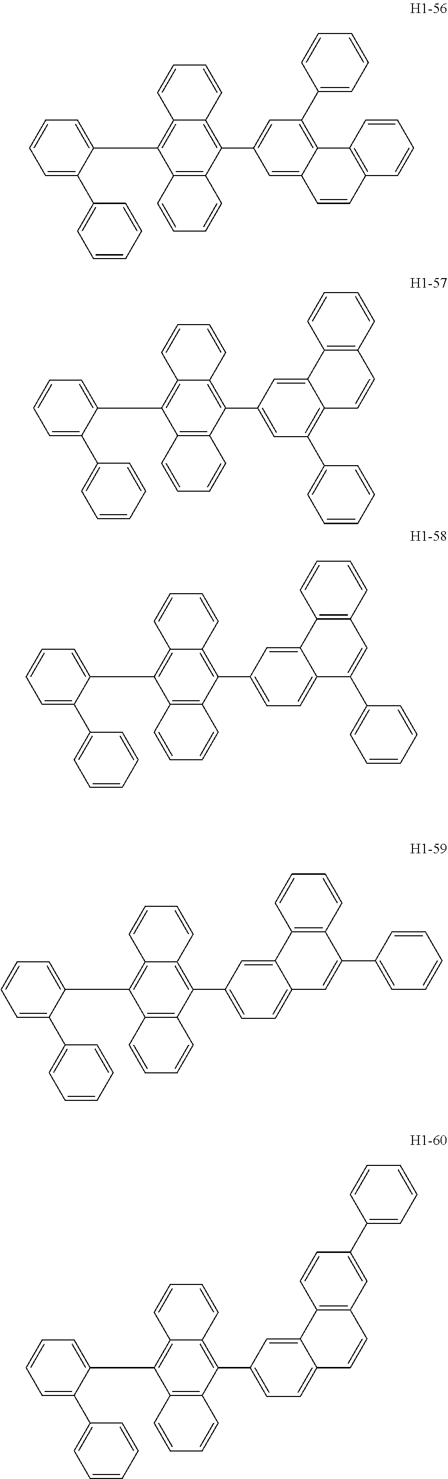

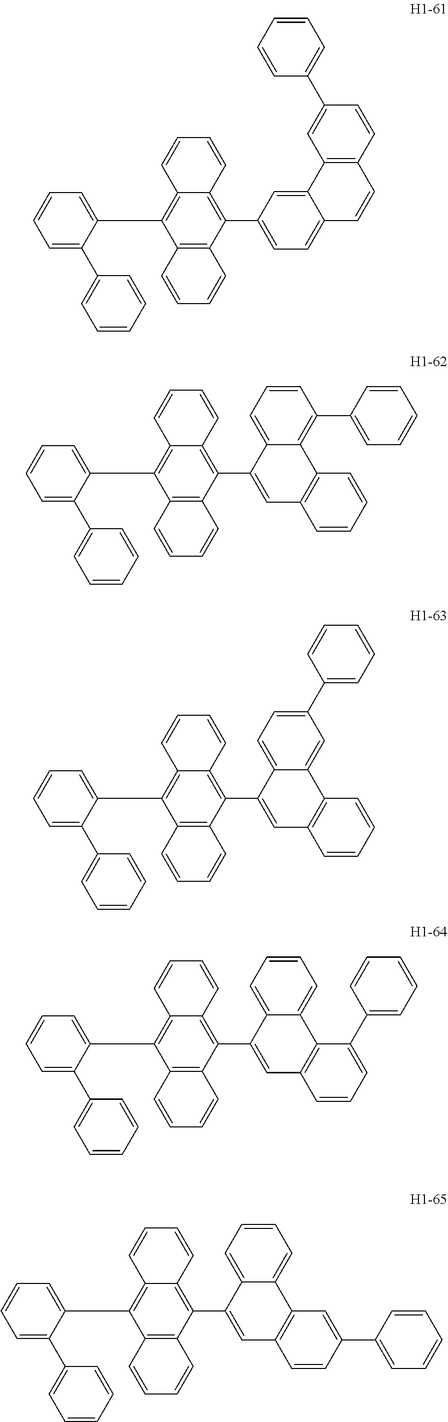

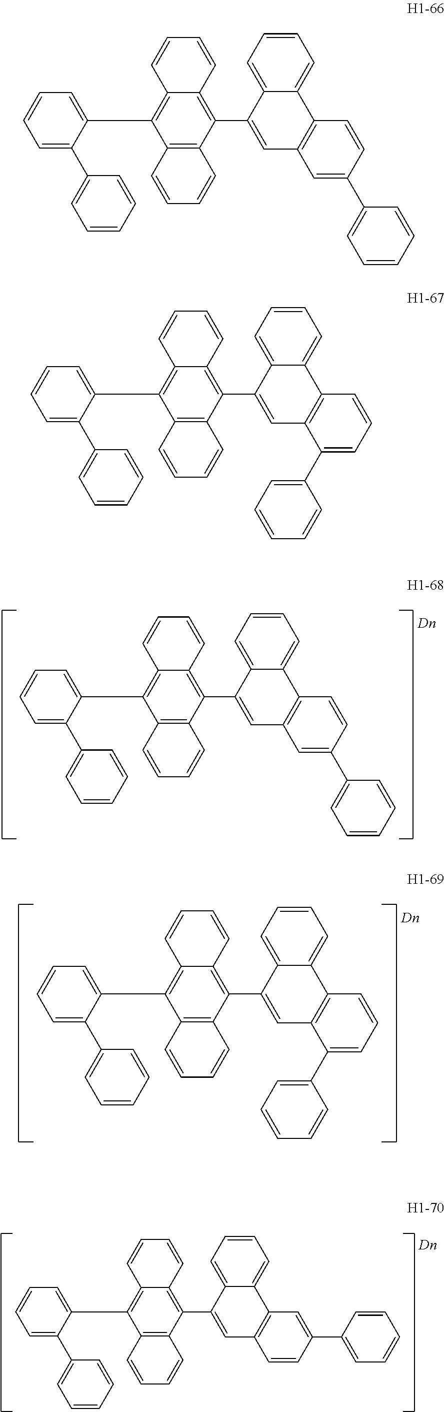

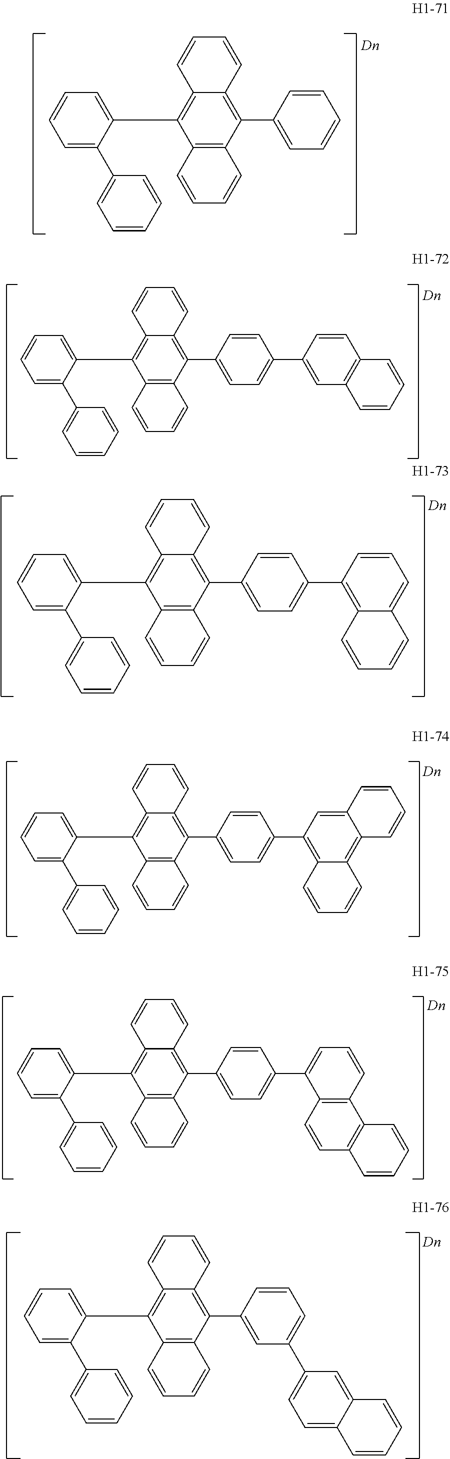

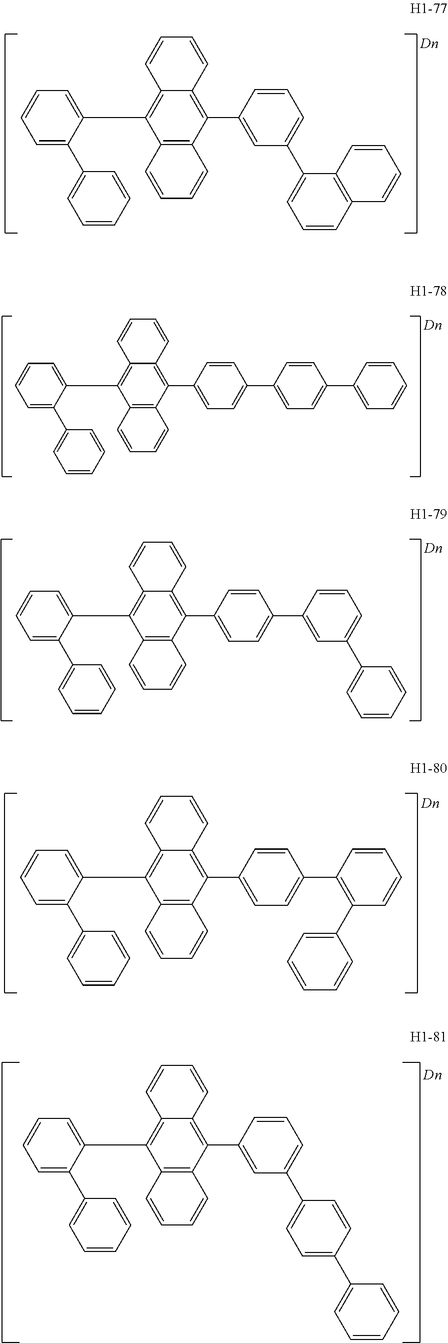

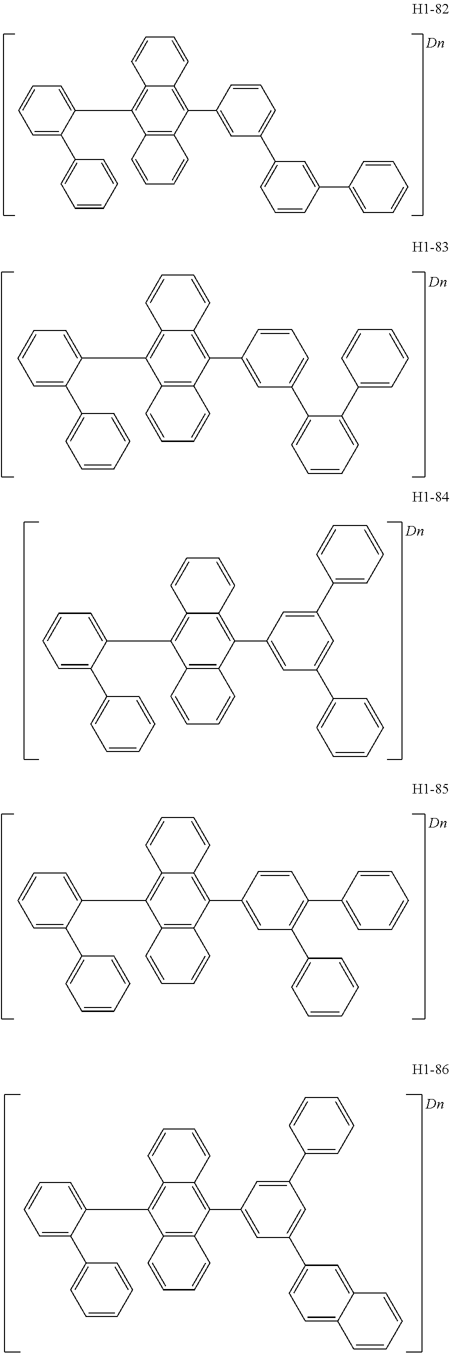

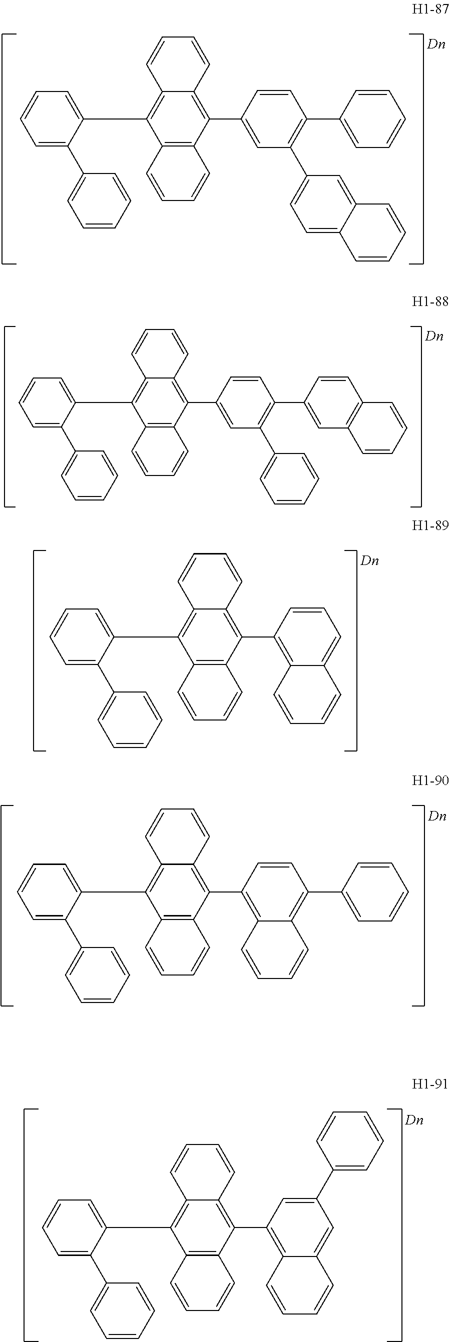

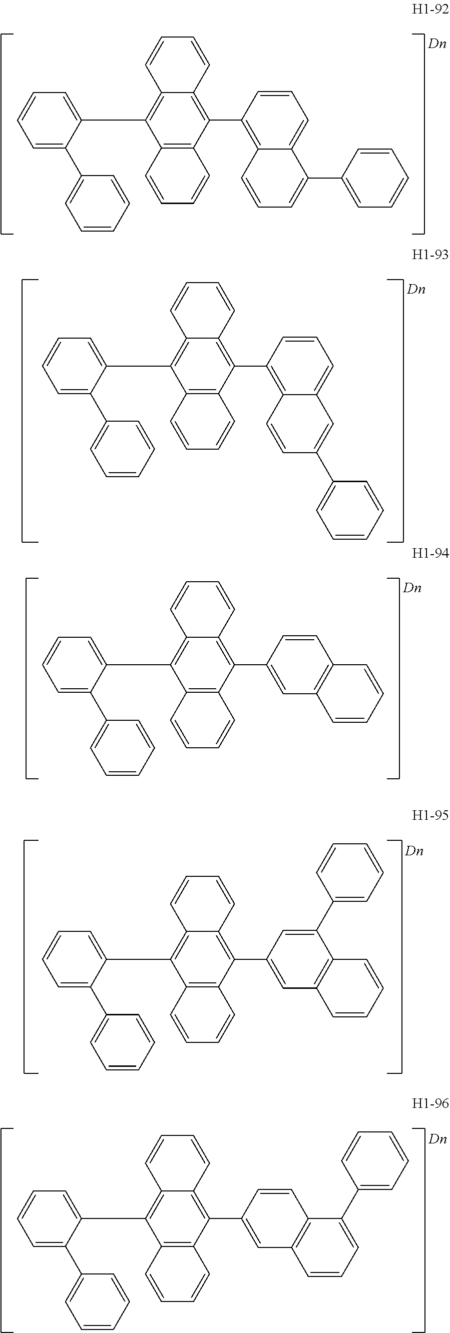

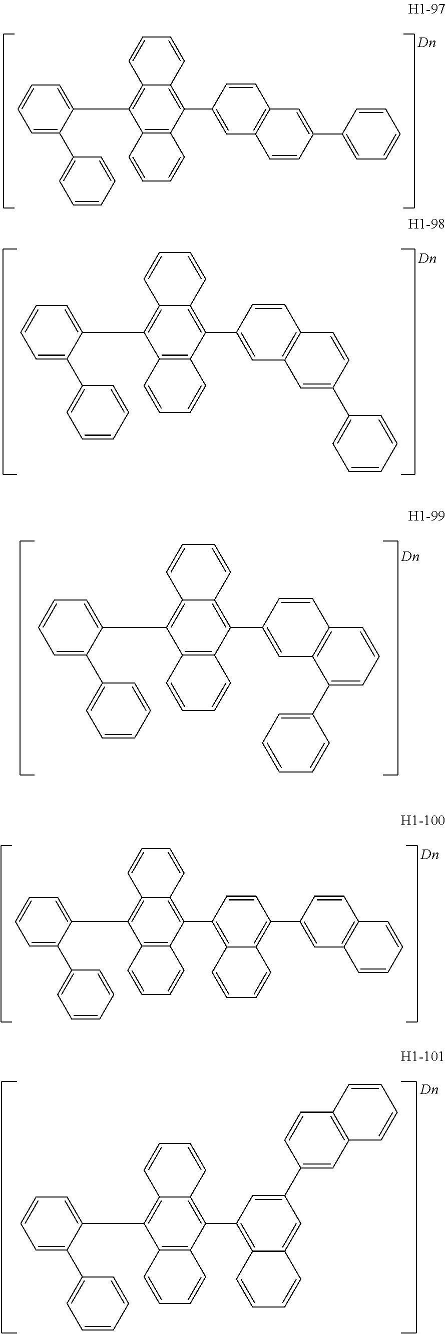

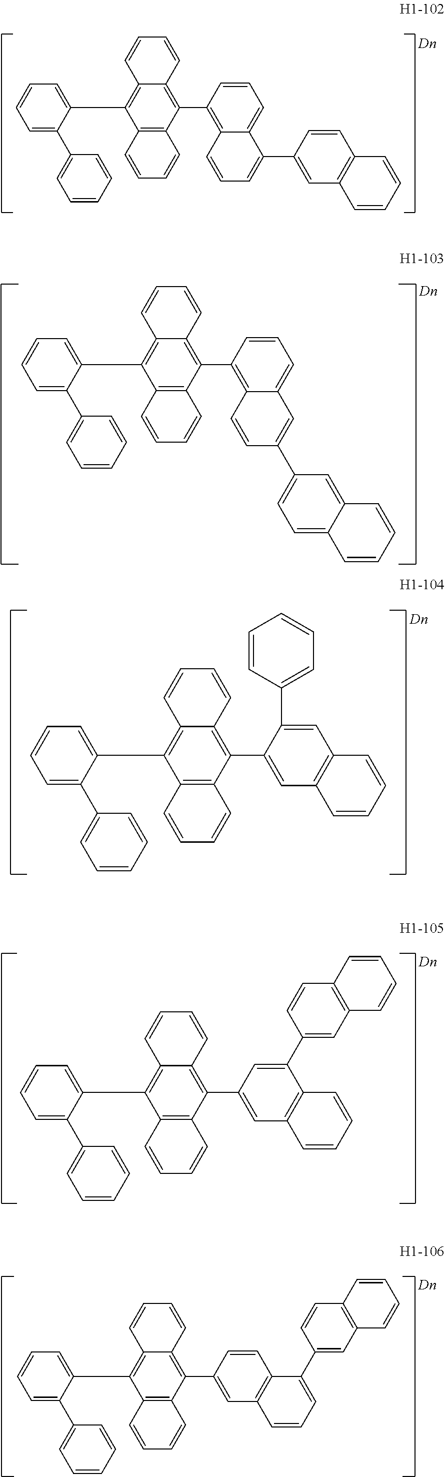

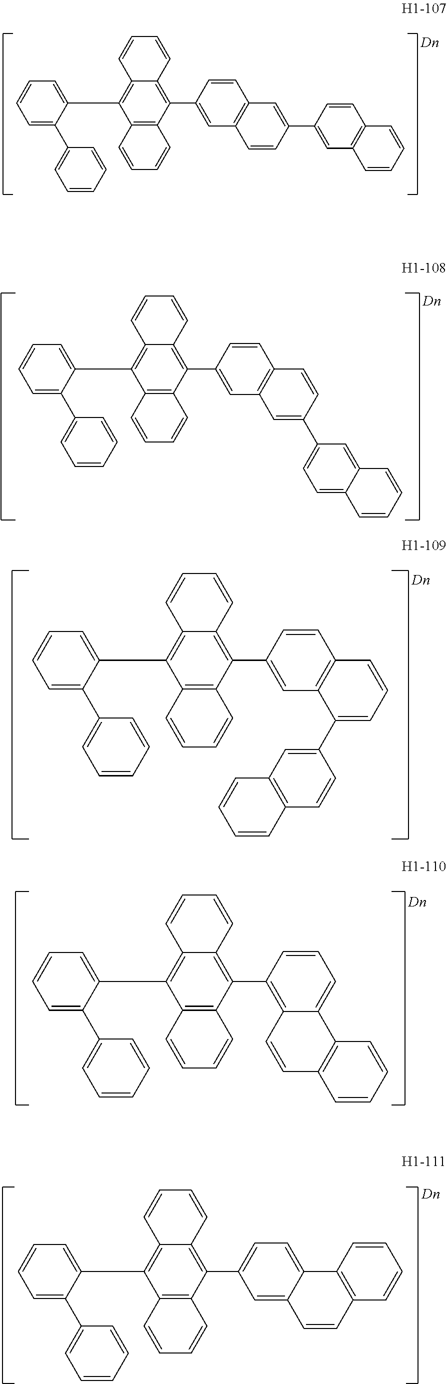

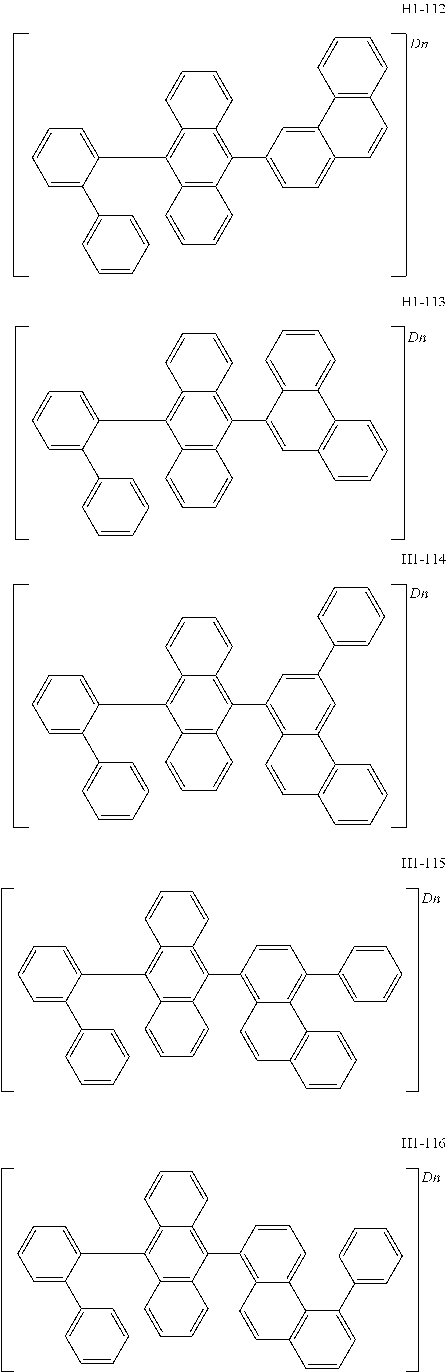

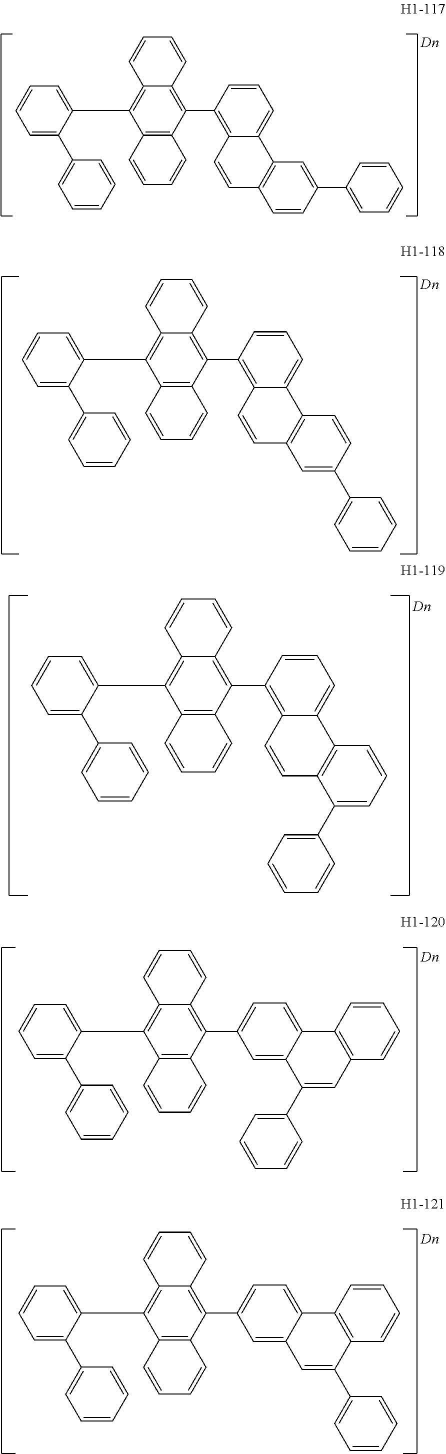

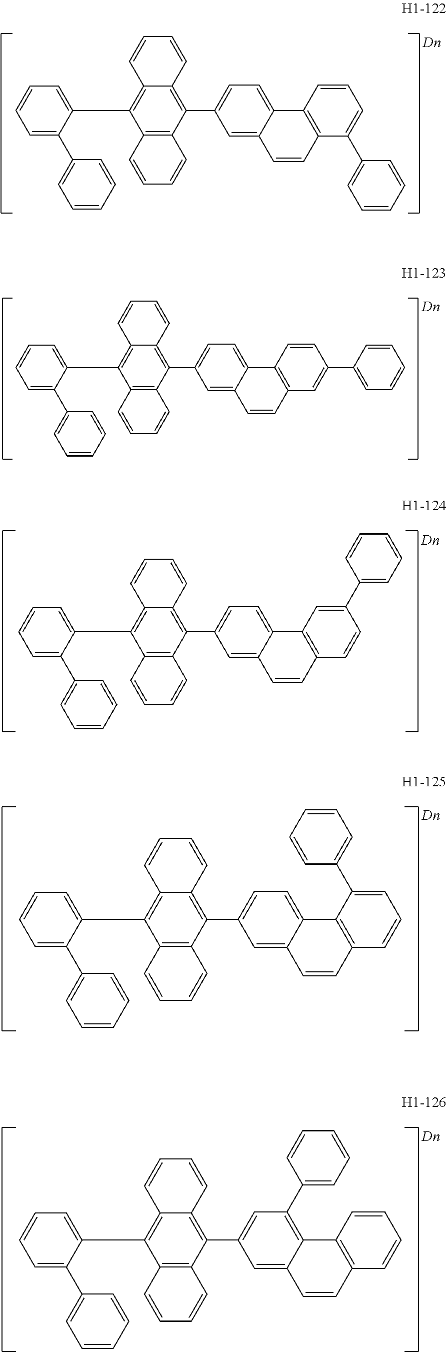

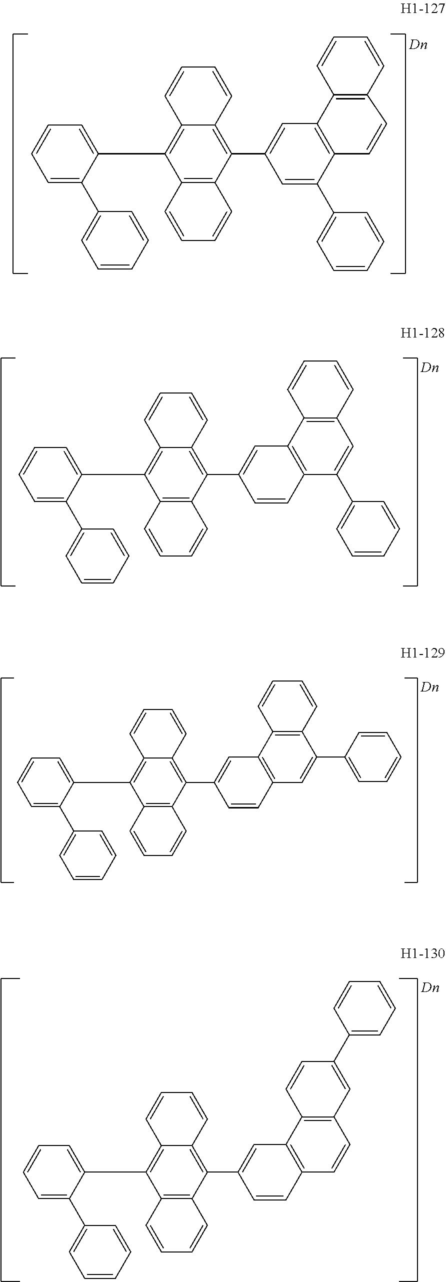

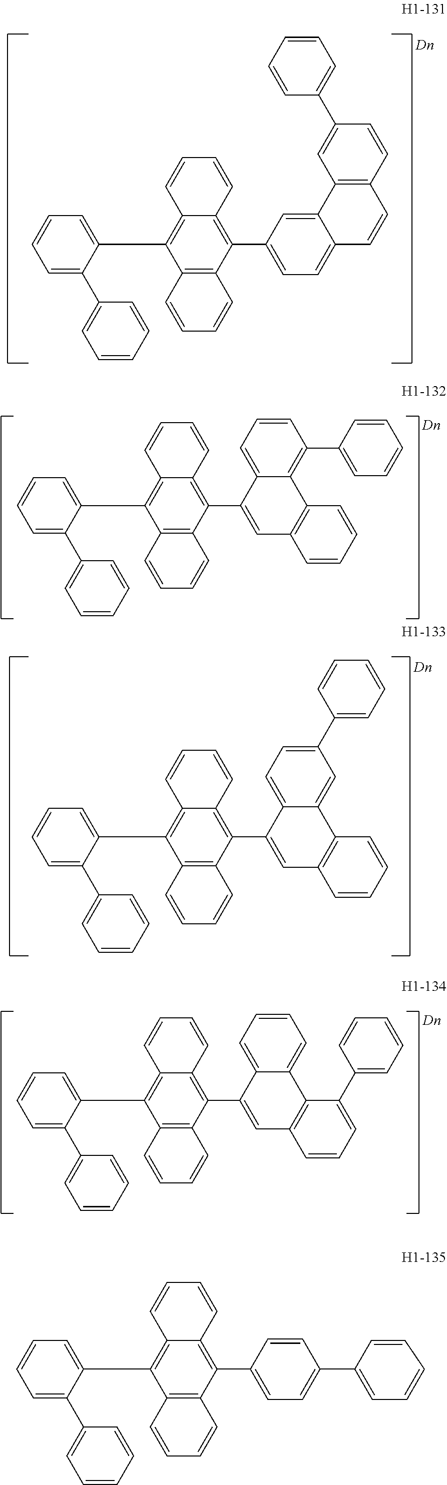

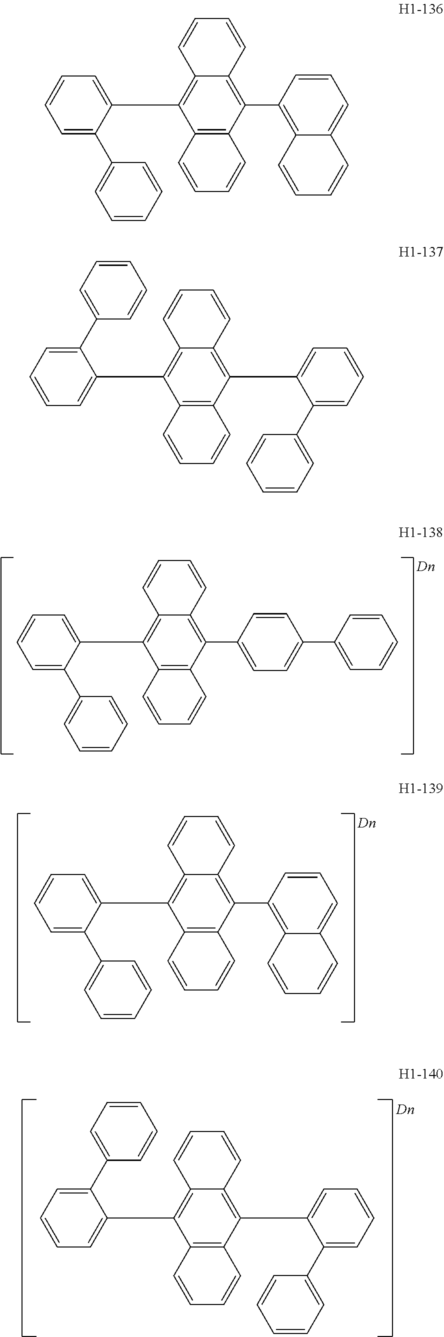

In the above compounds, Dₙ signifies that n hydrogens are replaced with deuterium, and n is an integer from 1 to the maximum number of hydrogens in the compound. Specifically, n is a minimum of 1 and an integer up to the maximum number of hydrogens in the compound. According to one embodiment of the present disclosure, in one compound represented by Formula 1, when deuterium is included, the deuterium substitution rate is preferably about 100% or less, more preferably about 95% or less, still more preferably about 90% or less, and still more preferably about 85% or less of the total number of hydrogens. The compound of Formula 1 substituted with the above deuterium substitution rate can increase the bond dissociation energy according to deuteration, thereby enhancing the stability of the compound, and an organic electroluminescent device comprising the compound can exhibit improved lifetime properties.

The compound represented by Formula 2 may be more specifically at least one selected from the group consisting of the following compounds, but is not limited thereto.

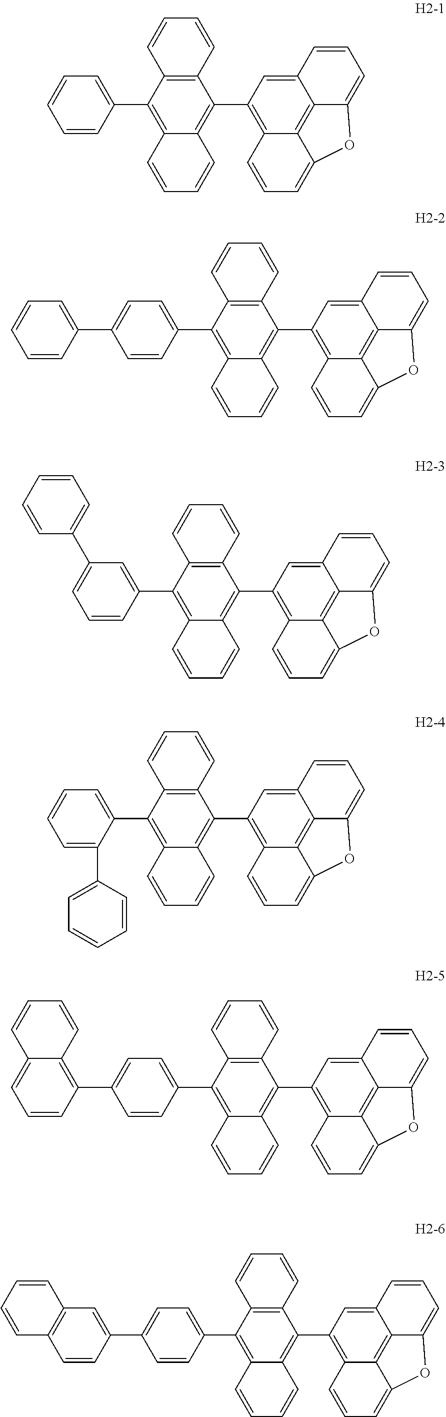 [SMILES: c1ccc(-c2c3ccccc3c(-c3cc4cccc5oc6cccc3c6c45)c3ccccc23)cc1 ; c1ccc(-c2ccc(-c3c4ccccc4c(-c4cc5cccc6oc7cccc4c7c56)c4ccccc34)cc2)cc1 ; c1ccc(-c2cccc(-c3c4ccccc4c(-c4cc5cccc6oc7cccc4c7c56)c4ccccc34)c2)cc1 ; c1ccc(-c2ccccc2-c2c3ccccc3c(-c3cc4cccc5oc6cccc3c6c45)c3ccccc23)cc1 ; c1ccc2c(-c3ccc(-c4c5ccccc5c(-c5cc6cccc7oc8cccc5c8c67)c5ccccc45)cc3)cccc2c1 ; c1ccc2cc(-c3ccc(-c4c5ccccc5c(-c5cc6cccc7oc8cccc5c8c67)c5ccccc45)cc3)ccc2c1]

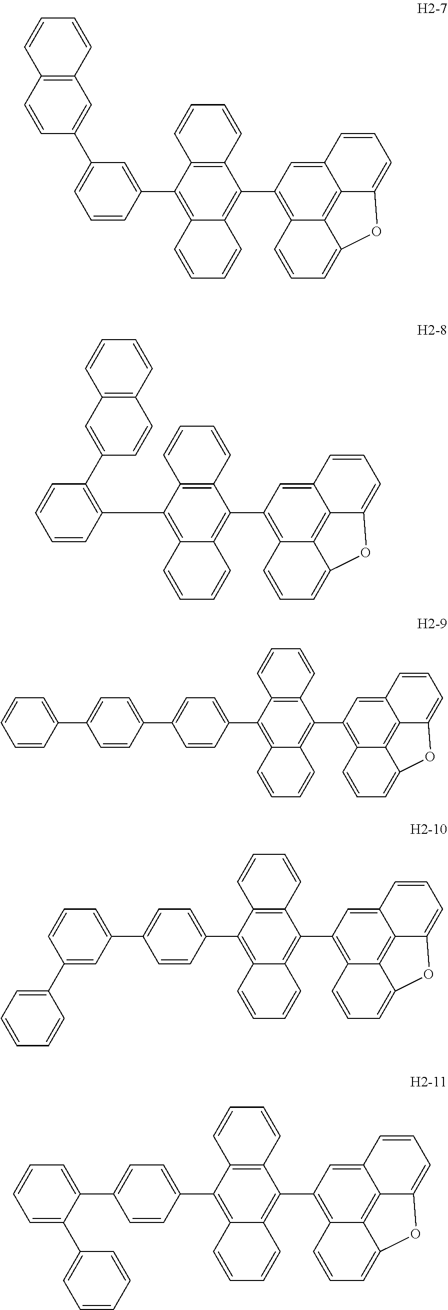 [SMILES: c1cc(-c2ccc3ccccc3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccccc3c2)c1 ; c1ccc(-c2ccc(-c3ccc(-c4c5ccccc5c(-c5cc6cccc7oc8cccc5c8c67)c5ccccc45)cc3)cc2)cc1 ; c1ccc(-c2cccc(-c3ccc(-c4c5ccccc5c(-c5cc6cccc7oc8cccc5c8c67)c5ccccc45)cc3)c2)cc1 ; c1ccc(-c2ccccc2-c2ccc(-c3c4ccccc4c(-c4cc5cccc6oc7cccc4c7c56)c4ccccc34)cc2)cc1]

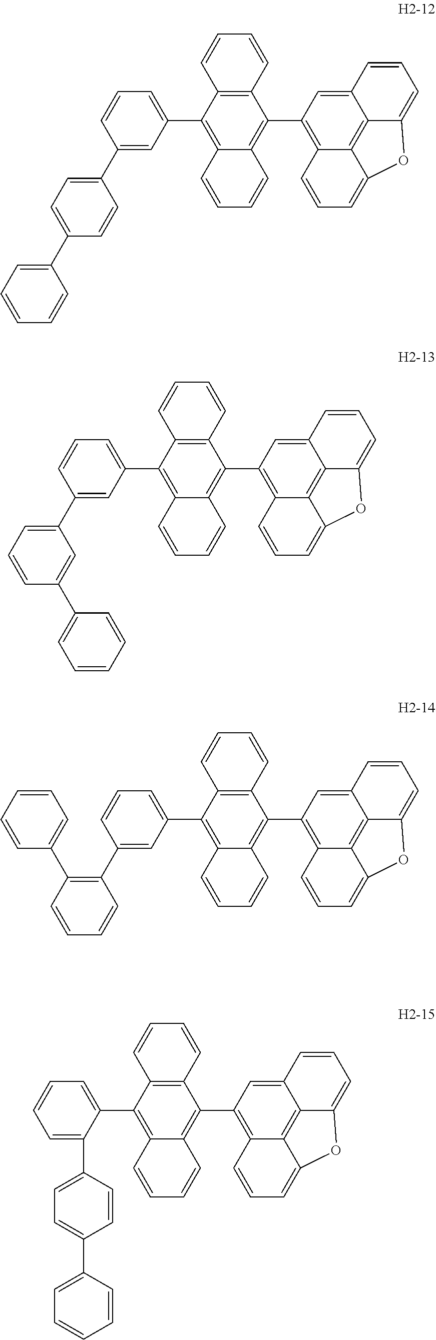 [SMILES: c1ccc(-c2ccc(-c3cccc(-c4c5ccccc5c(-c5cc6cccc7oc8cccc5c8c67)c5ccccc45)c3)cc2)cc1 ; c1ccc(-c2cccc(-c3cccc(-c4c5ccccc5c(-c5cc6cccc7oc8cccc5c8c67)c5ccccc45)c3)c2)cc1 ; c1ccc(-c2ccccc2-c2cccc(-c3c4ccccc4c(-c4cc5cccc6oc7cccc4c7c56)c4ccccc34)c2)cc1 ; c1ccc(-c2ccc(-c3ccccc3-c3c4ccccc4c(-c4cc5cccc6oc7cccc4c7c56)c4ccccc34)cc2)cc1]

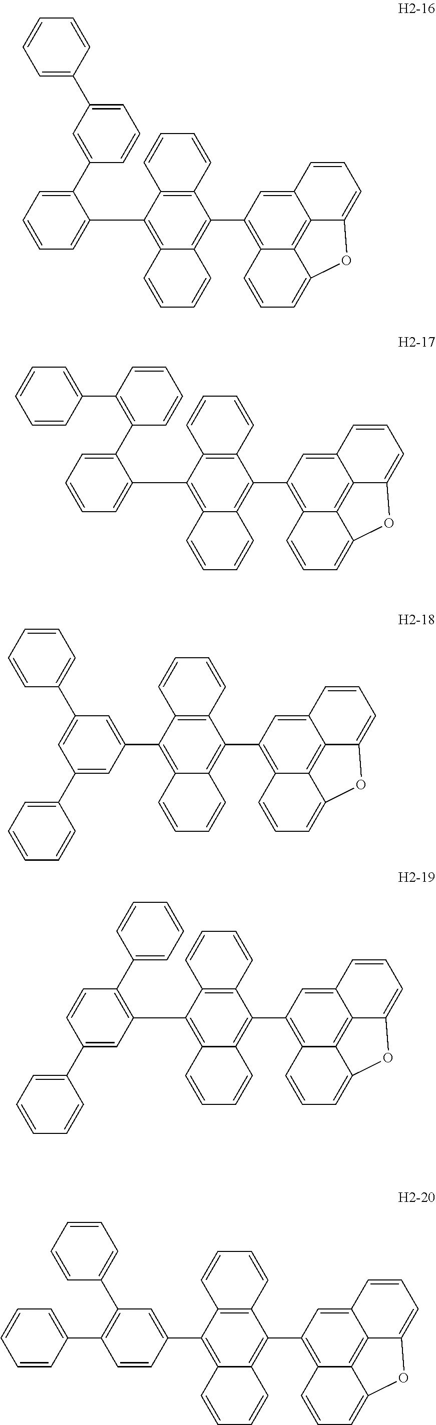 [SMILES: c1ccc(-c2cccc(-c3ccccc3-c3c4ccccc4c(-c4cc5cccc6oc7cccc4c7c56)c4ccccc34)c2)cc1 ; c1ccc(-c2ccccc2-c2ccccc2-c2c3ccccc3c(-c3cc4cccc5oc6cccc3c6c45)c3ccccc23)cc1 ; c1ccc(-c2cc(-c3ccccc3)cc(-c3c4ccccc4c(-c4cc5cccc6oc7cccc4c7c56)c4ccccc34)c2)cc1 ; c1ccc(-c2ccc(-c3ccccc3)c(-c3c4ccccc4c(-c4cc5cccc6oc7cccc4c7c56)c4ccccc34)c2)cc1 ; c1ccc(-c2ccc(-c3c4ccccc4c(-c4cc5cccc6oc7cccc4c7c56)c4ccccc34)cc2-c2ccccc2)cc1]

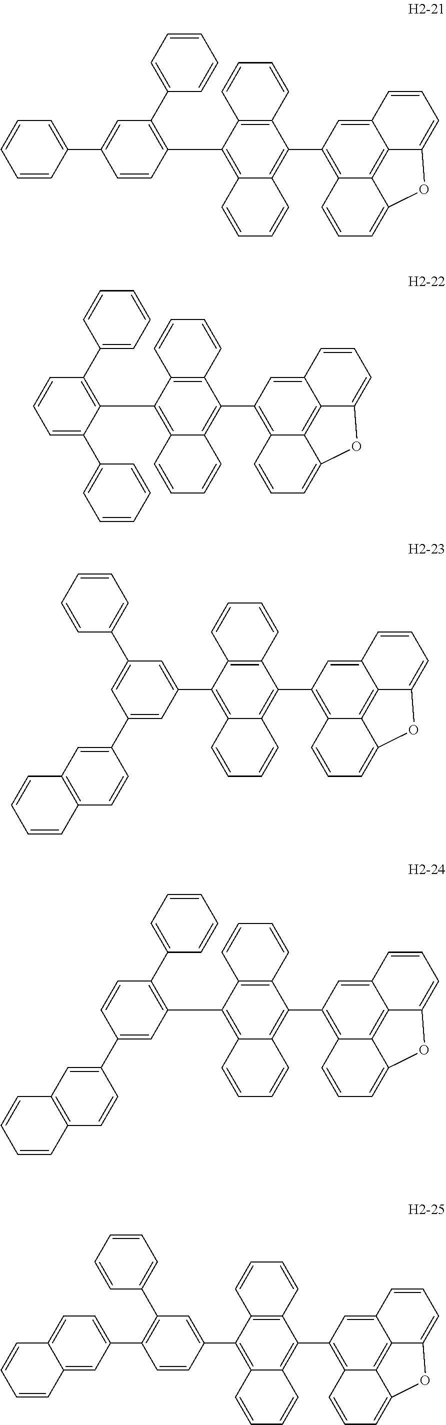 [SMILES: c1ccc(-c2ccc(-c3c4ccccc4c(-c4cc5cccc6oc7cccc4c7c56)c4ccccc34)c(-c3ccccc3)c2)cc1 ; c1ccc(-c2cccc(-c3ccccc3)c2-c2c3ccccc3c(-c3cc4cccc5oc6cccc3c6c45)c3ccccc23)cc1 ; c1ccc(-c2cc(-c3ccc4ccccc4c3)cc(-c3c4ccccc4c(-c4cc5cccc6oc7cccc4c7c56)c4ccccc34)c2)cc1 ; c1ccc(-c2ccc(-c3ccc4ccccc4c3)cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)ccc2-c2ccc3ccccc3c2)cc1]

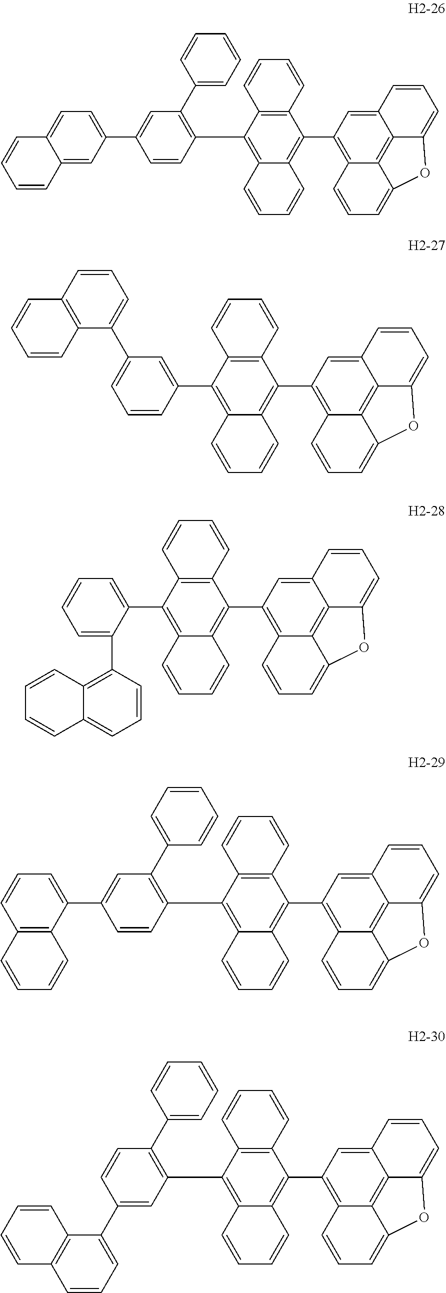 [SMILES: c1ccc(-c2cc(-c3ccc4ccccc4c3)ccc2-c2c3ccccc3c(-c3cc4cccc5oc6cccc3c6c45)c3ccccc23)cc1 ; c1cc(-c2cccc3ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2cccc3ccccc23)c1 ; c1ccc(-c2cc(-c3cccc4ccccc34)ccc2-c2c3ccccc3c(-c3cc4cccc5oc6cccc3c6c45)c3ccccc23)cc1 ; c1ccc(-c2ccc(-c3cccc4ccccc34)cc2-c2c3ccccc3c(-c3cc4cccc5oc6cccc3c6c45)c3ccccc23)cc1]

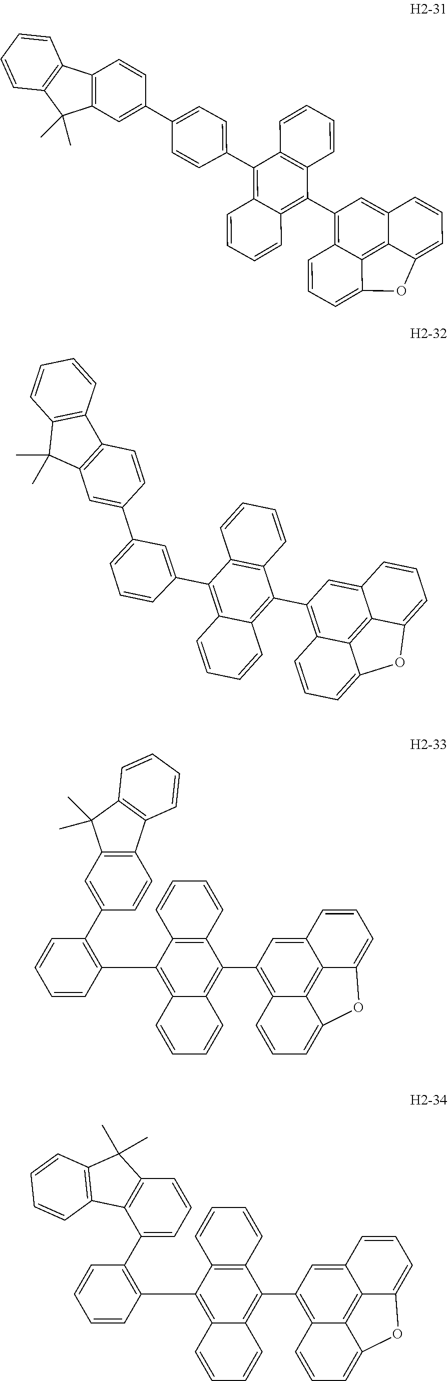 [SMILES: CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21 ; CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21]

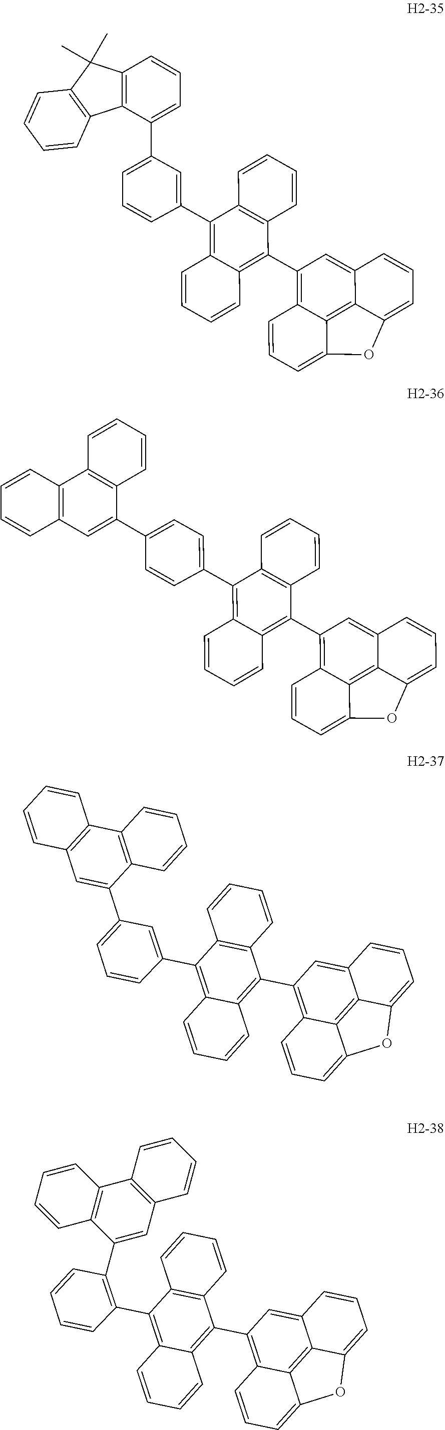 [SMILES: CC1(C)c2ccccc2-c2c(-c3cccc(-c4c5ccccc5c(-c5cc6cccc7oc8cccc5c8c67)c5ccccc45)c3)cccc21 ; c1ccc2c(c1)cc(-c1ccc(-c3c4ccccc4c(-c4cc5cccc6oc7cccc4c7c56)c4ccccc34)cc1)c1ccccc12 ; c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1]

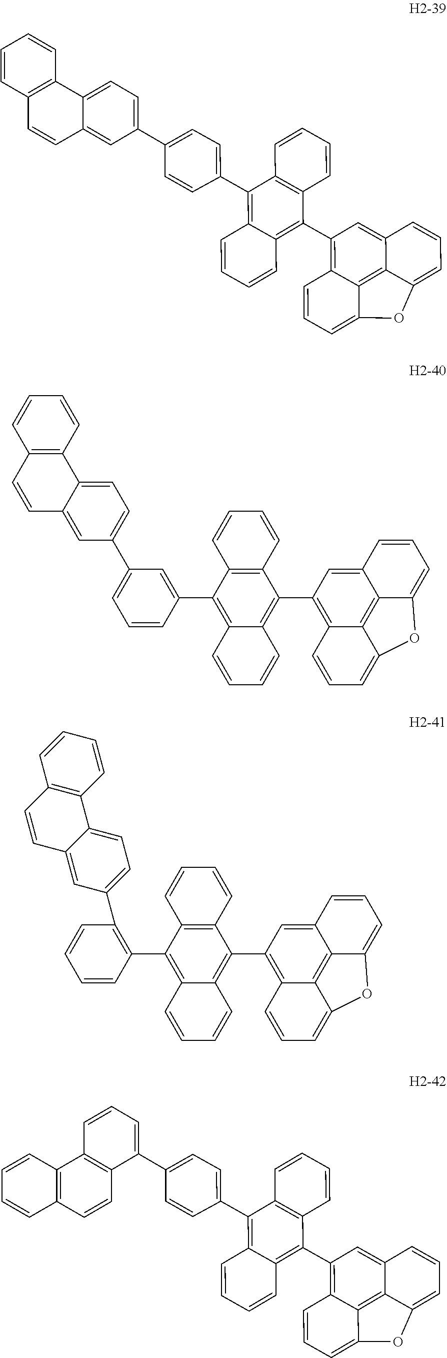 [SMILES: c1ccc2c(c1)ccc1cc(-c3ccc(-c4c5ccccc5c(-c5cc6cccc7oc8cccc5c8c67)c5ccccc45)cc3)ccc12 ; c1cc(-c2ccc3c(ccc4ccccc43)c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(ccc4ccccc43)c2)c1 ; c1ccc2c(c1)ccc1c(-c3ccc(-c4c5ccccc5c(-c5cc6cccc7oc8cccc5c8c67)c5ccccc45)cc3)cccc12]

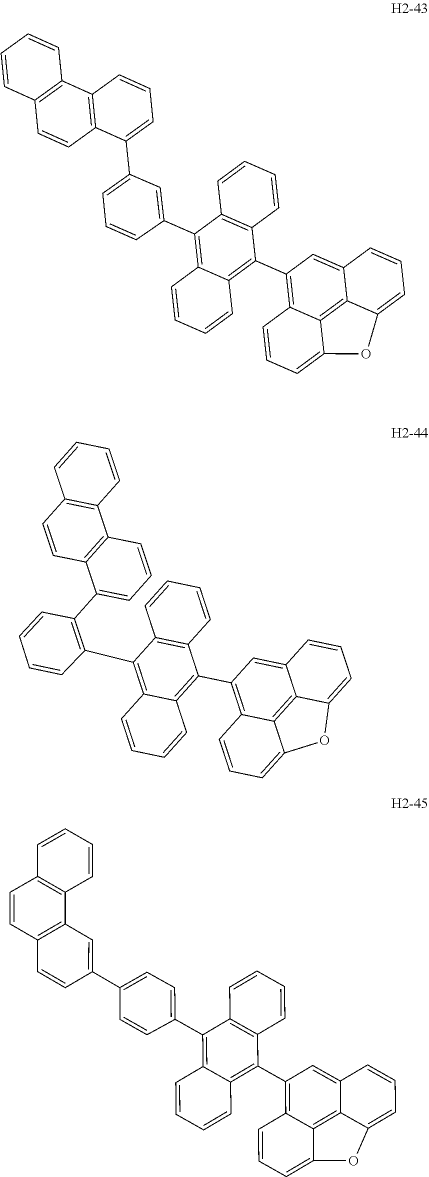 [SMILES: c1cc(-c2c3ccccc3c(-c3cc4cccc5oc6cccc3c6c45)c3ccccc23)cc(-c2cccc3c2ccc2ccccc23)c1 ; c1ccc(-c2cccc3c2ccc2ccccc23)c(-c2c3ccccc3c(-c3cc4cccc5oc6cccc3c6c45)c3ccccc23)c1 ; c1ccc2c(c1)ccc1ccc(-c3ccc(-c4c5ccccc5c(-c5cc6cccc7oc8cccc5c8c67)c5ccccc45)cc3)cc12]

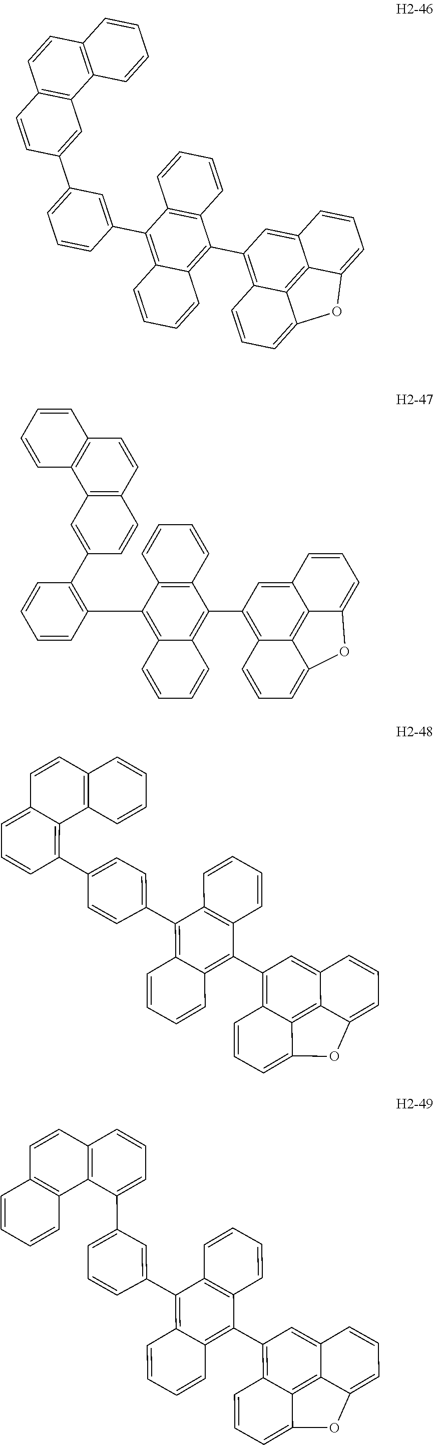 [SMILES: c1cc(-c2ccc3ccc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccc4ccccc4c3c2)c1 ; c1ccc2c(c1)ccc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3ccc4ccccc4c23)c1]

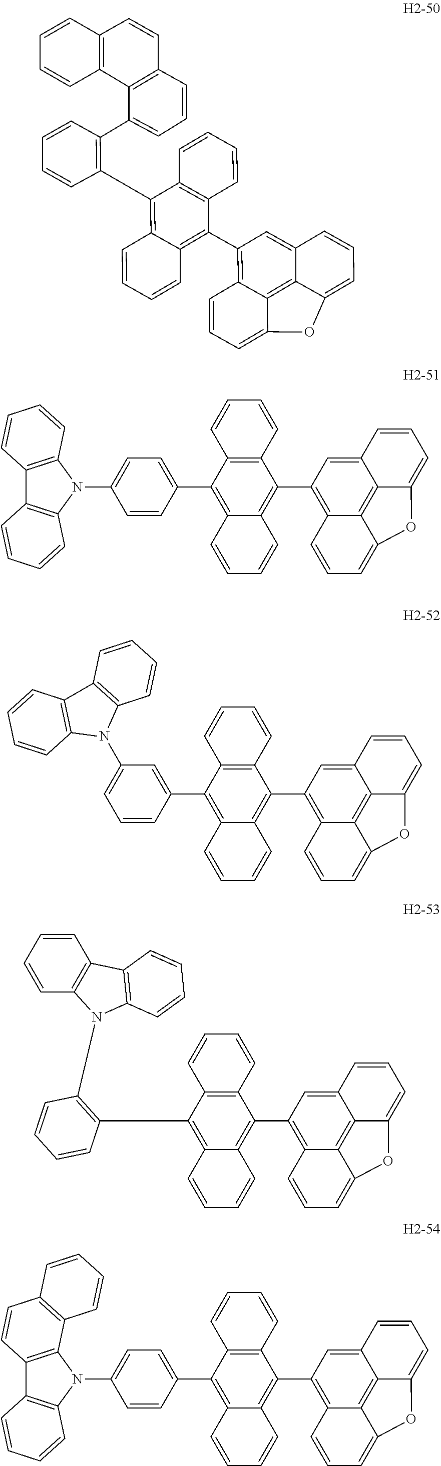 [SMILES: c1ccc(-c2cccc3ccc4ccccc4c23)c(-c2c3ccccc3c(-c3cc4cccc5oc6cccc3c6c45)c3ccccc23)c1 ; c1cc2cc(-c3c4ccccc4c(-c4ccc(-n5c6ccccc6c6ccccc65)cc4)c4ccccc34)c3cccc4oc(c1)c2c43 ; c1cc(-c2c3ccccc3c(-c3cc4cccc5oc6cccc3c6c45)c3ccccc23)cc(-n2c3ccccc3c3ccccc32)c1 ; c1ccc(-n2c3ccccc3c3ccccc32)c(-c2c3ccccc3c(-c3cc4cccc5oc6cccc3c6c45)c3ccccc23)c1 ; c1ccc2c(c1)ccc1c3ccccc3n(-c3ccc(-c4c5ccccc5c(-c5cc6cccc7oc8cccc5c8c67)c5ccccc45)cc3)c21]

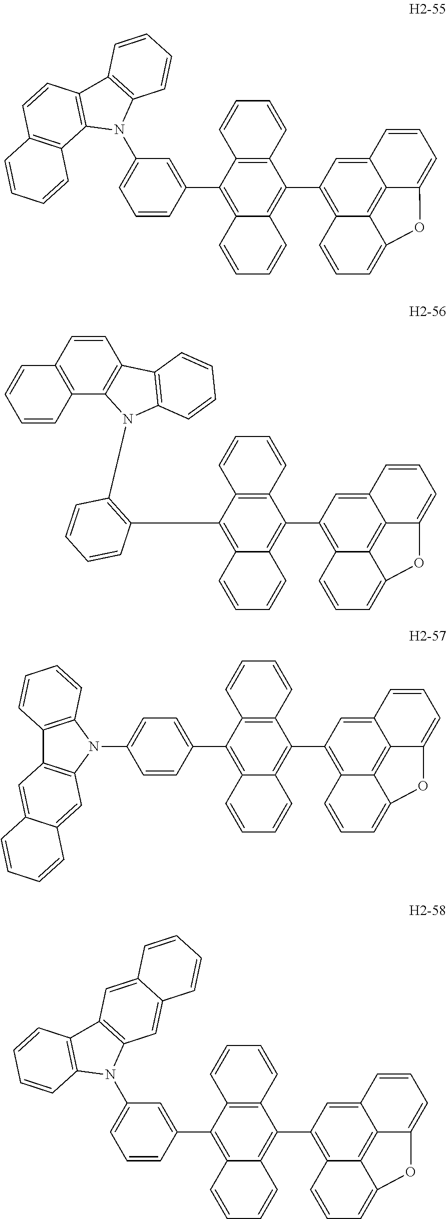 [SMILES: c1cc(-c2c3ccccc3c(-c3cc4cccc5oc6cccc3c6c45)c3ccccc23)cc(-n2c3ccccc3c3ccc4ccccc4c32)c1 ; c1ccc(-n2c3ccccc3c3ccc4ccccc4c32)c(-c2c3ccccc3c(-c3cc4cccc5oc6cccc3c6c45)c3ccccc23)c1 ; c1ccc2cc3c(cc2c1)c1ccccc1n3-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3cc4ccccc4cc32)c1]

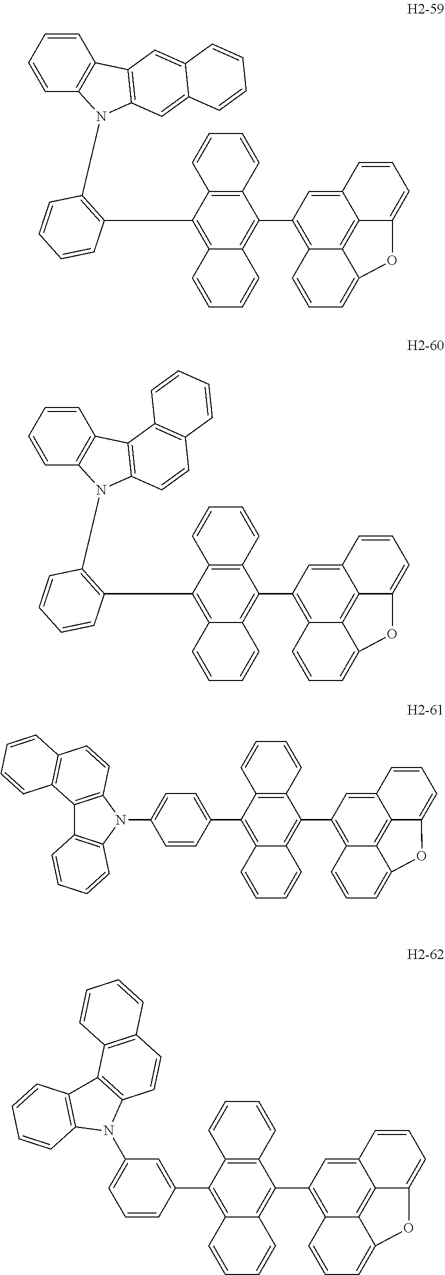 [SMILES: c1ccc(-n2c3ccccc3c3cc4ccccc4cc32)c(-c2c3ccccc3c(-c3cc4cccc5oc6cccc3c6c45)c3ccccc23)c1 ; c1ccc(-n2c3ccccc3c3c4ccccc4ccc32)c(-c2c3ccccc3c(-c3cc4cccc5oc6cccc3c6c45)c3ccccc23)c1 ; c1ccc2c(c1)ccc1c2c2ccccc2n1-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3c4ccccc4ccc32)c1]

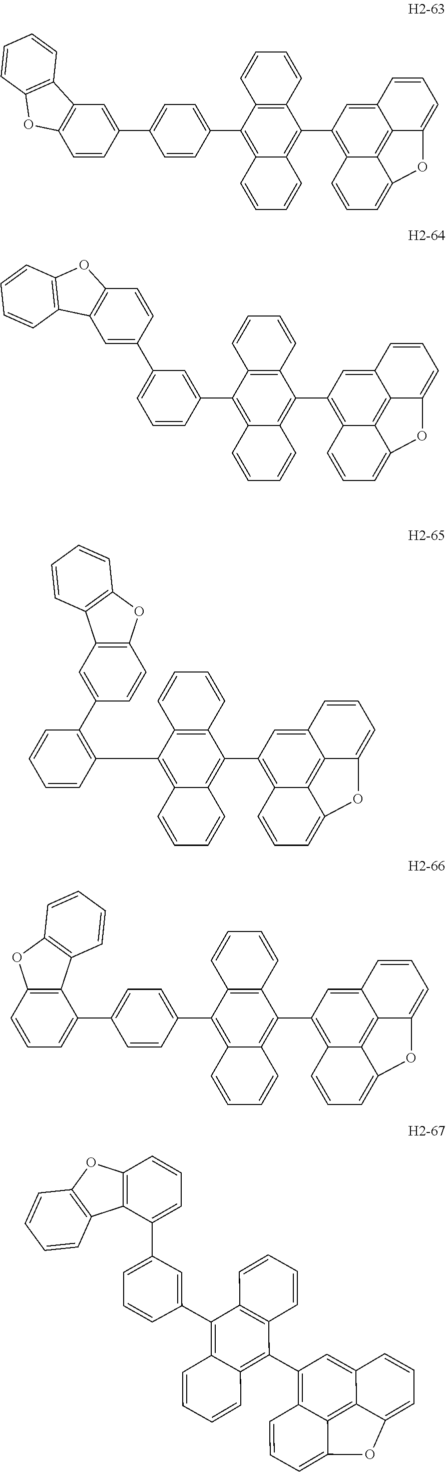 [SMILES: c1ccc2c(c1)oc1ccc(-c3ccc(-c4c5ccccc5c(-c5cc6cccc7oc8cccc5c8c67)c5ccccc45)cc3)cc12 ; c1cc(-c2ccc3oc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3oc4ccccc4c3c2)c1 ; c1ccc2c(c1)oc1cccc(-c3ccc(-c4c5ccccc5c(-c5cc6cccc7oc8cccc5c8c67)c5ccccc45)cc3)c12 ; c1cc(-c2c3ccccc3c(-c3cc4cccc5oc6cccc3c6c45)c3ccccc23)cc(-c2cccc3oc4ccccc4c23)c1]

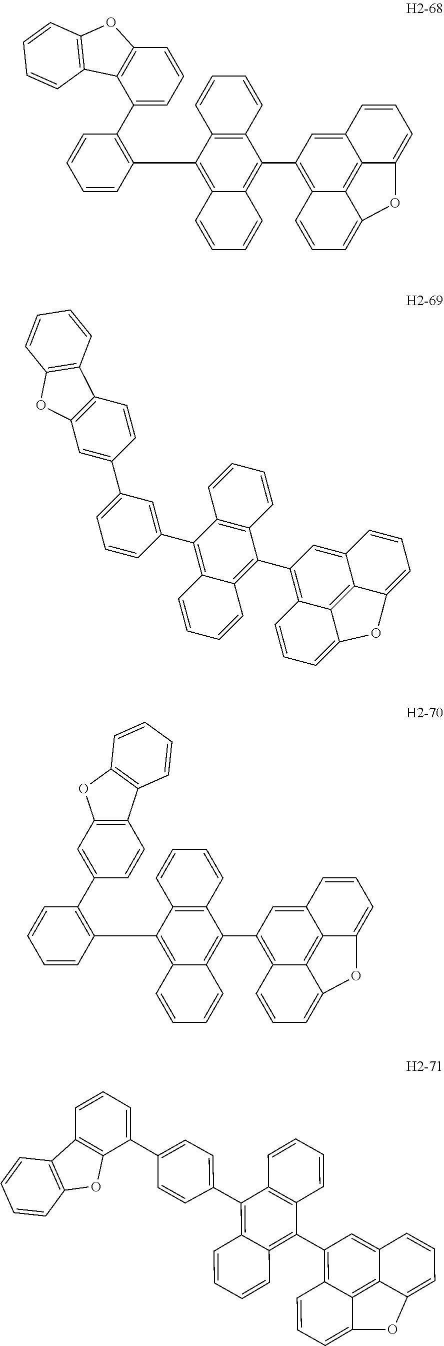 [SMILES: c1ccc(-c2cccc3oc4ccccc4c23)c(-c2c3ccccc3c(-c3cc4cccc5oc6cccc3c6c45)c3ccccc23)c1 ; c1cc(-c2ccc3c(c2)oc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7oc8cccc5c8c67)c5ccccc45)cc3)cccc12]

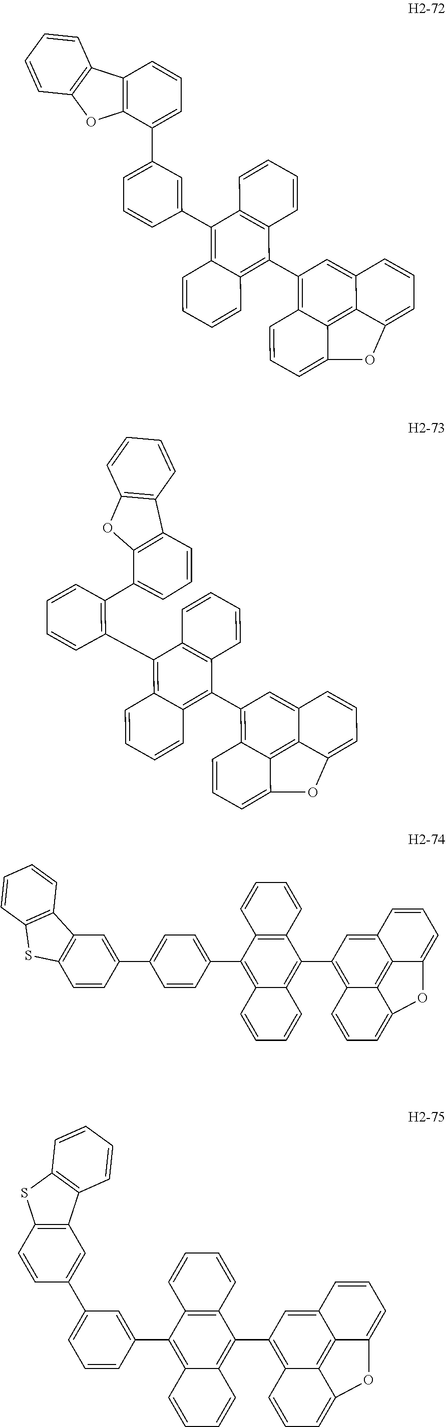 [SMILES: c1cc(-c2c3ccccc3c(-c3cc4cccc5oc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5oc6cccc3c6c45)c3ccccc23)c1 ; c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1]

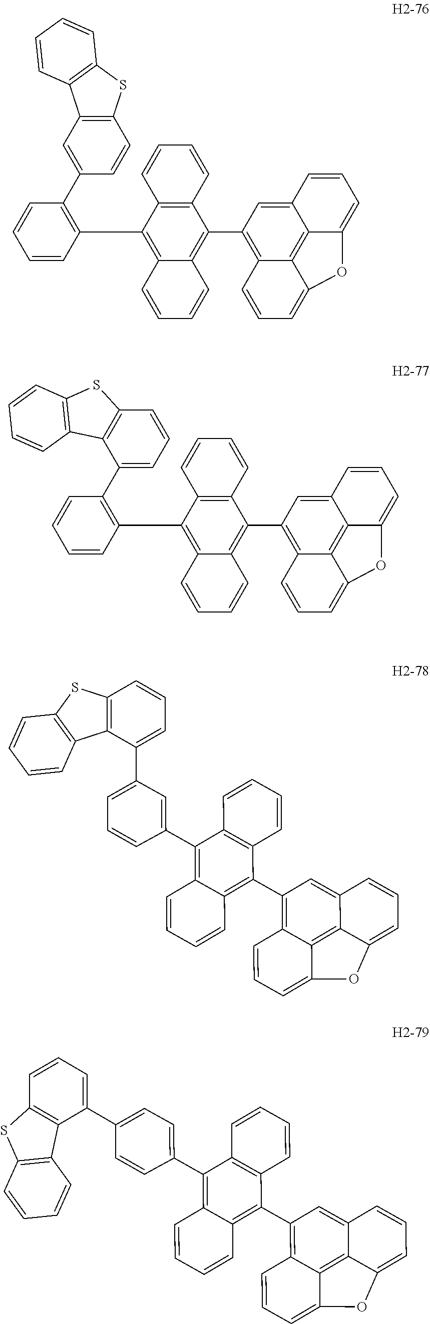 [SMILES: c1ccc(-c2c3ccccc3c(-c3cc4cccc5oc6cccc3c6c45)c3ccccc23)c(-c2ccc3sc4ccccc4c3c2)c1 ; c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1 ; c1cc2cc(-c3c4ccccc4c(-c4ccc(-c5cccc6sc7ccccc7c56)cc4)c4ccccc34)c3cccc4oc(c1)c2c43]

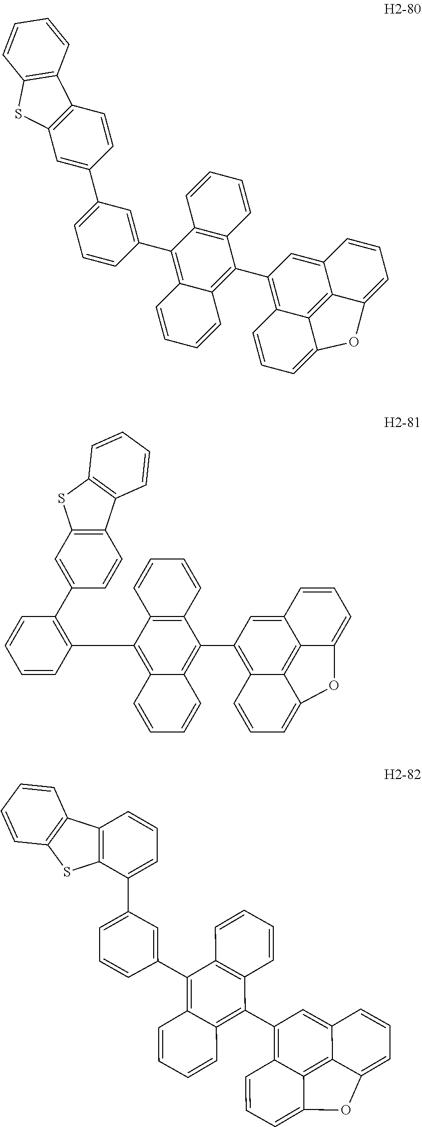 [SMILES: c1cc(-c2ccc3c(c2)sc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1]

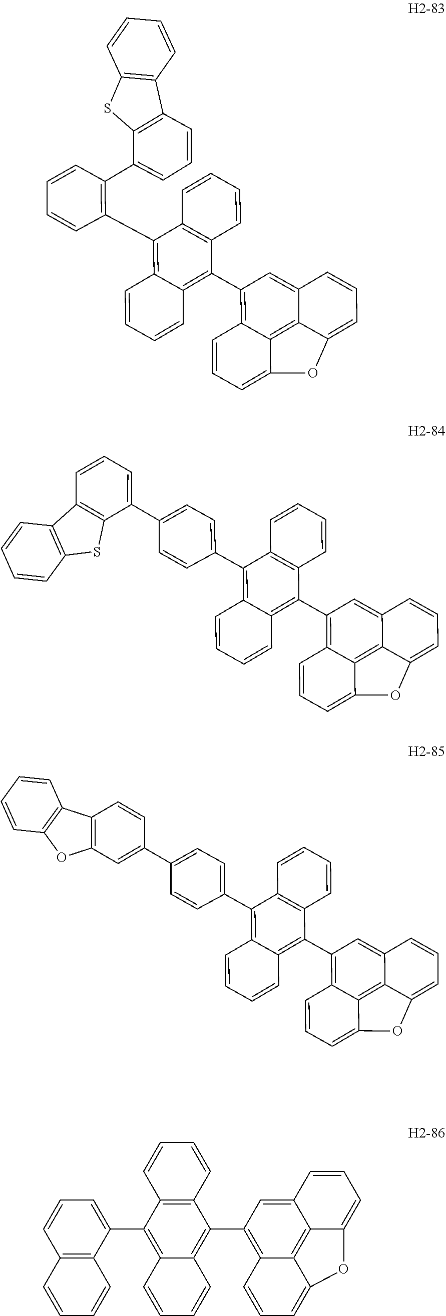 [SMILES: c1ccc(-c2cccc3c2sc2ccccc23)c(-c2c3ccccc3c(-c3cc4cccc5oc6cccc3c6c45)c3ccccc23)c1 ; c1cc2cc(-c3c4ccccc4c(-c4ccc(-c5cccc6c5sc5ccccc56)cc4)c4ccccc34)c3cccc4oc(c1)c2c43 ; c1ccc2c(c1)oc1cc(-c3ccc(-c4c5ccccc5c(-c5cc6cccc7oc8cccc5c8c67)c5ccccc45)cc3)ccc12 ; c1ccc2c(-c3c4ccccc4c(-c4cc5cccc6oc7cccc4c7c56)c4ccccc34)cccc2c1]

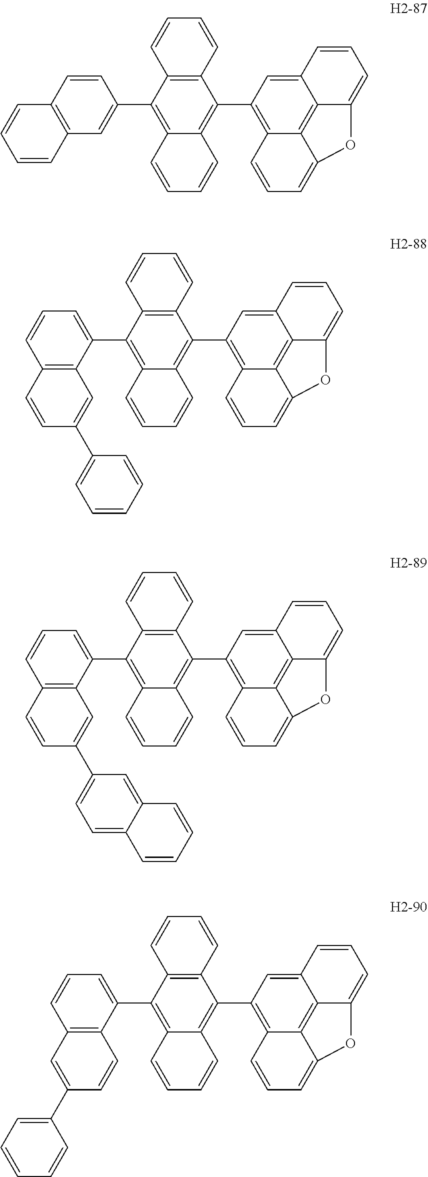 [SMILES: c1ccc2cc(-c3c4ccccc4c(-c4cc5cccc6oc7cccc4c7c56)c4ccccc34)ccc2c1 ; c1ccc(-c2ccc3cccc(-c4c5ccccc5c(-c5cc6cccc7oc8cccc5c8c67)c5ccccc45)c3c2)cc1 ; c1ccc2cc(-c3ccc4cccc(-c5c6ccccc6c(-c6cc7cccc8oc9cccc6c9c78)c6ccccc56)c4c3)ccc2c1 ; c1ccc(-c2ccc3c(-c4c5ccccc5c(-c5cc6cccc7oc8cccc5c8c67)c5ccccc45)cccc3c2)cc1]

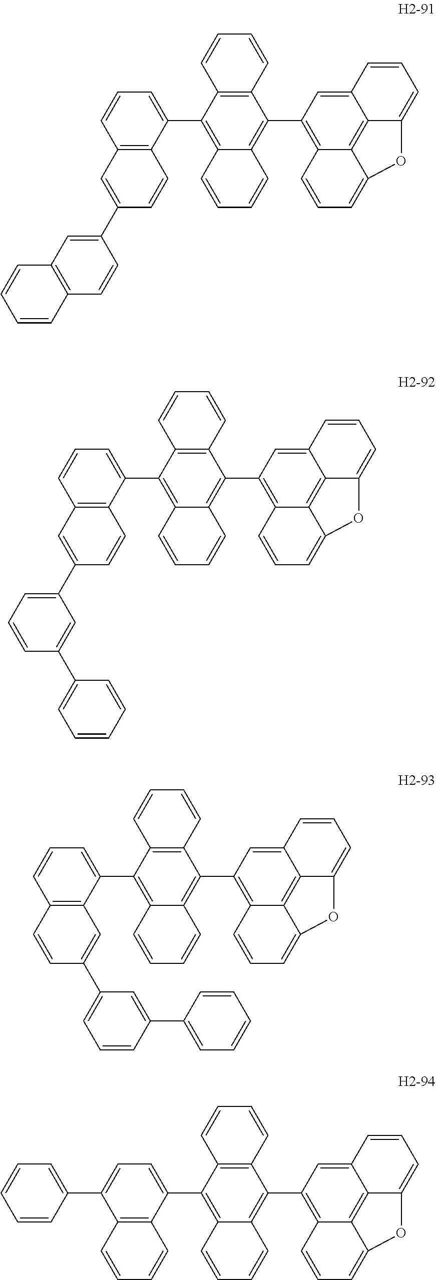 [SMILES: c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1 ; c1ccc(-c2ccc(-c3c4ccccc4c(-c4cc5cccc6oc7cccc4c7c56)c4ccccc34)c3ccccc23)cc1]

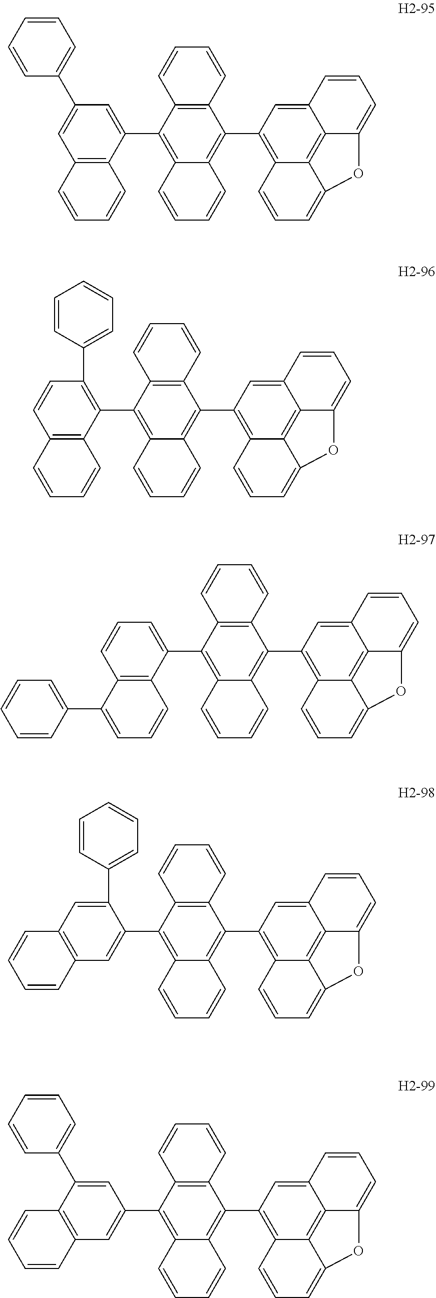 [SMILES: c1ccc(-c2cc(-c3c4ccccc4c(-c4cc5cccc6oc7cccc4c7c56)c4ccccc34)c3ccccc3c2)cc1 ; c1ccc(-c2ccc3ccccc3c2-c2c3ccccc3c(-c3cc4cccc5oc6cccc3c6c45)c3ccccc23)cc1 ; c1ccc(-c2cccc3c(-c4c5ccccc5c(-c5cc6cccc7oc8cccc5c8c67)c5ccccc45)cccc23)cc1 ; c1ccc(-c2cc3ccccc3cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)cc3ccccc23)cc1]

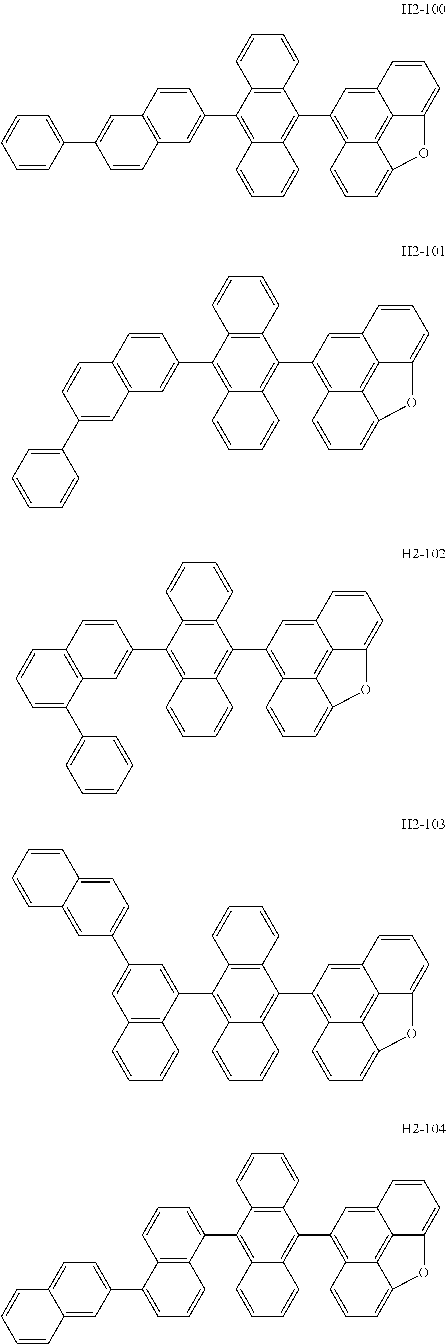 [SMILES: c1ccc(-c2ccc3cc(-c4c5ccccc5c(-c5cc6cccc7oc8cccc5c8c67)c5ccccc45)ccc3c2)cc1 ; c1ccc(-c2ccc3ccc(-c4c5ccccc5c(-c5cc6cccc7oc8cccc5c8c67)c5ccccc45)cc3c2)cc1 ; c1ccc(-c2cccc3ccc(-c4c5ccccc5c(-c5cc6cccc7oc8cccc5c8c67)c5ccccc45)cc23)cc1 ; c1ccc2cc(-c3cc(-c4c5ccccc5c(-c5cc6cccc7oc8cccc5c8c67)c5ccccc45)c4ccccc4c3)ccc2c1 ; c1ccc2cc(-c3cccc4c(-c5c6ccccc6c(-c6cc7cccc8oc9cccc6c9c78)c6ccccc56)cccc34)ccc2c1]

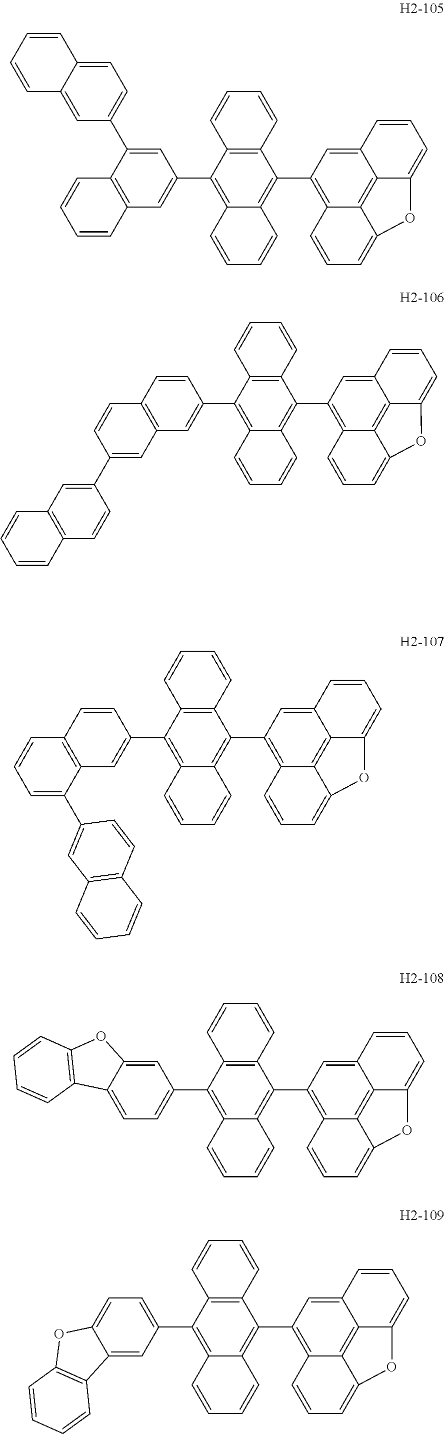 [SMILES: c1ccc2cc(-c3cc(-c4c5ccccc5c(-c5cc6cccc7oc8cccc5c8c67)c5ccccc45)cc4ccccc34)ccc2c1 ; c1ccc2cc(-c3ccc4ccc(-c5c6ccccc6c(-c6cc7cccc8oc9cccc6c9c78)c6ccccc56)cc4c3)ccc2c1 ; c1ccc2cc(-c3cccc4ccc(-c5c6ccccc6c(-c6cc7cccc8oc9cccc6c9c78)c6ccccc56)cc34)ccc2c1 ; c1ccc2c(c1)oc1cc(-c3c4ccccc4c(-c4cc5cccc6oc7cccc4c7c56)c4ccccc34)ccc12 ; c1ccc2c(c1)oc1ccc(-c3c4ccccc4c(-c4cc5cccc6oc7cccc4c7c56)c4ccccc34)cc12]

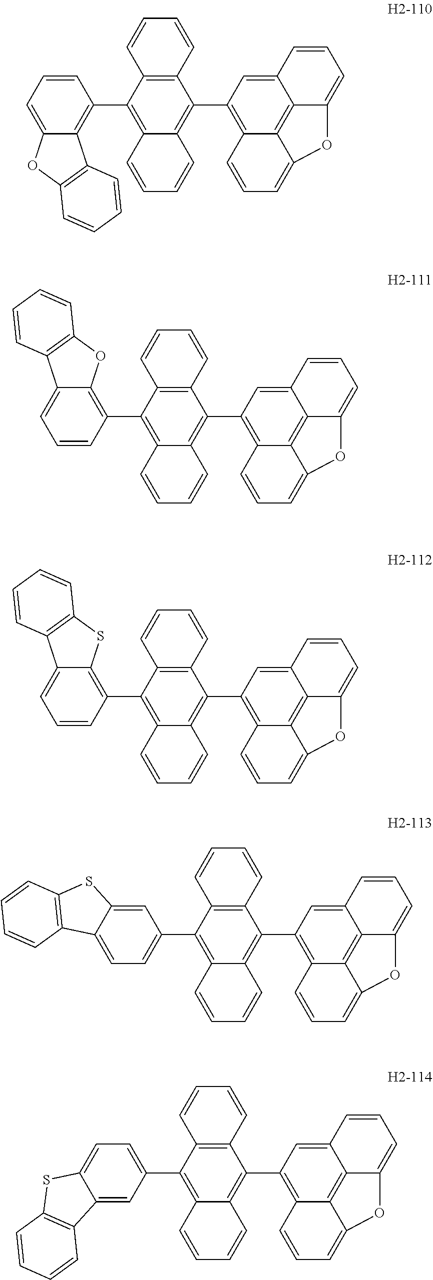 [SMILES: c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43]

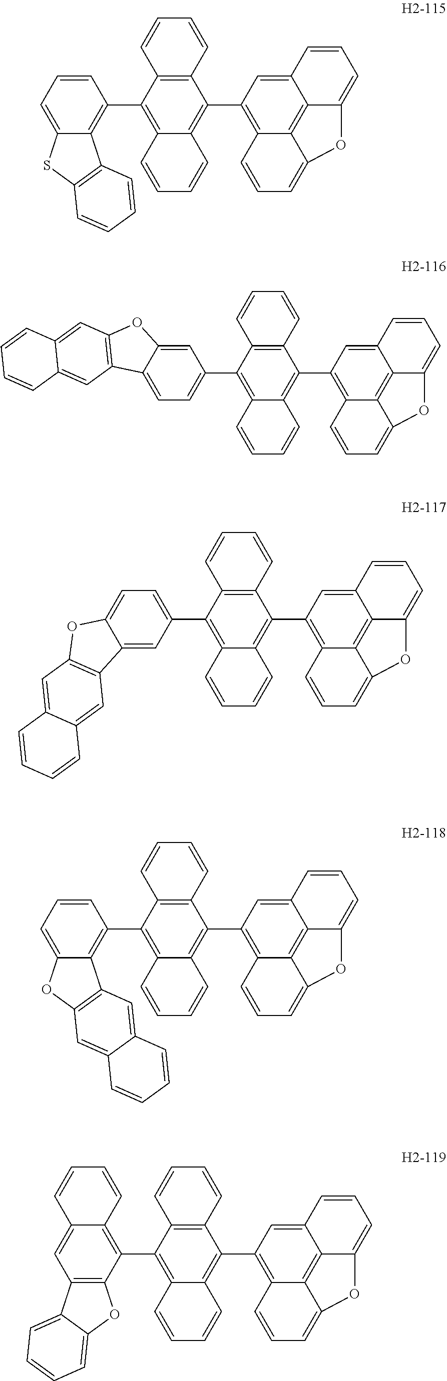 [SMILES: c1cc2cc(-c3c4ccccc4c(-c4cccc5sc6ccccc6c45)c4ccccc34)c3cccc4oc(c1)c2c43 ; c1ccc2cc3c(cc2c1)oc1cc(-c2c4ccccc4c(-c4cc5cccc6oc7cccc4c7c56)c4ccccc24)ccc13 ; c1ccc2cc3c(cc2c1)oc1ccc(-c2c4ccccc4c(-c4cc5cccc6oc7cccc4c7c56)c4ccccc24)cc13 ; c1ccc2cc3c(cc2c1)oc1cccc(-c2c4ccccc4c(-c4cc5cccc6oc7cccc4c7c56)c4ccccc24)c13 ; c1ccc2c(-c3c4ccccc4c(-c4cc5cccc6oc7cccc4c7c56)c4ccccc34)c3oc4ccccc4c3cc2c1]

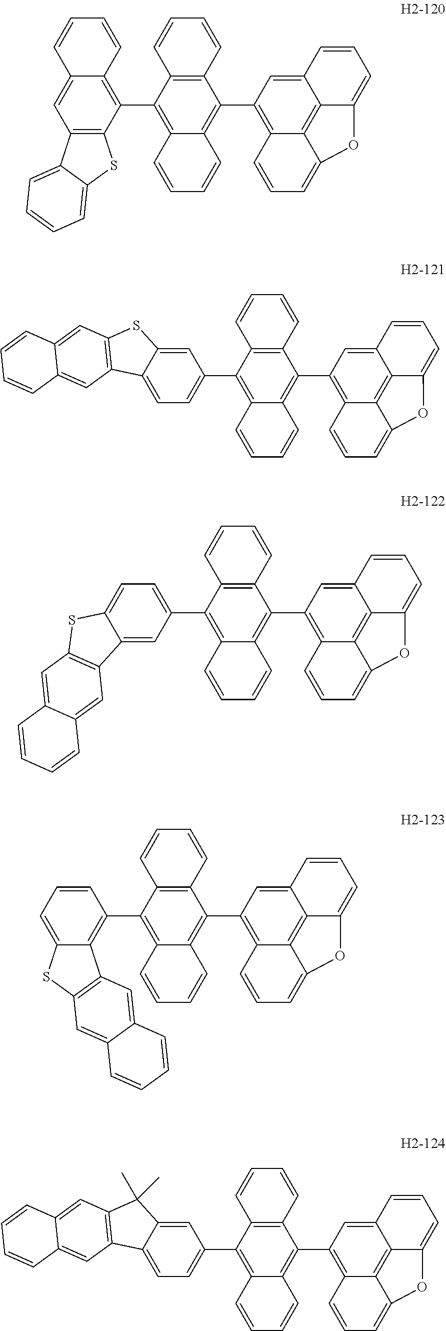 [SMILES: c1ccc2c(-c3c4ccccc4c(-c4cc5cccc6oc7cccc4c7c56)c4ccccc34)c3sc4ccccc4c3cc2c1 ; c1ccc2cc3c(cc2c1)sc1cc(-c2c4ccccc4c(-c4cc5cccc6oc7cccc4c7c56)c4ccccc24)ccc13 ; c1ccc2cc3c(cc2c1)sc1ccc(-c2c4ccccc4c(-c4cc5cccc6oc7cccc4c7c56)c4ccccc24)cc13 ; c1ccc2cc3c(cc2c1)sc1cccc(-c2c4ccccc4c(-c4cc5cccc6oc7cccc4c7c56)c4ccccc24)c13 ; CC1(C)c2cc(-c3c4ccccc4c(-c4cc5cccc6oc7cccc4c7c56)c4ccccc34)ccc2-c2cc3ccccc3cc21]

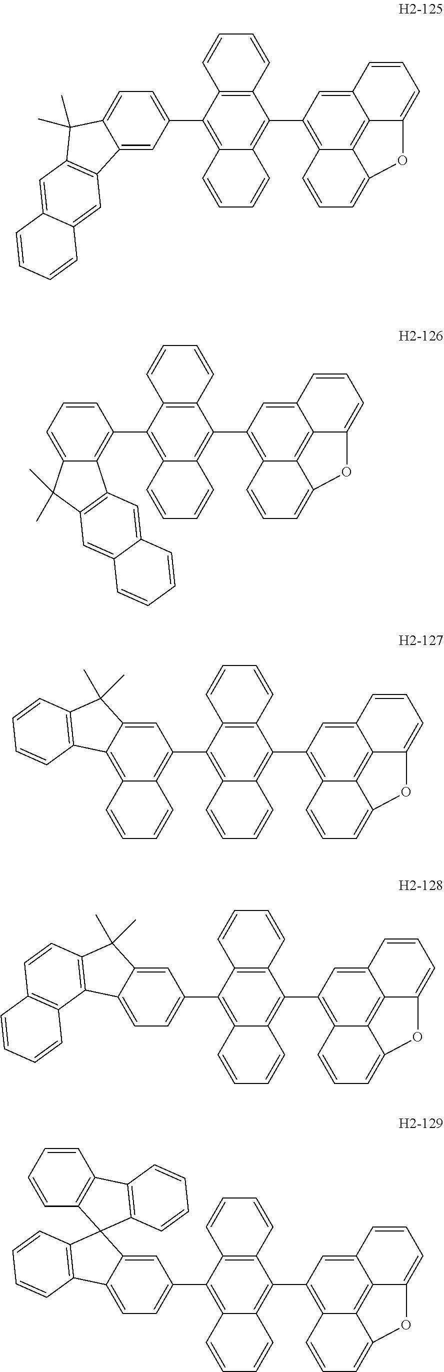 [SMILES: CC1(C)c2ccc(-c3c4ccccc4c(-c4cc5cccc6oc7cccc4c7c56)c4ccccc34)cc2-c2cc3ccccc3cc21 ; CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6oc7cccc4c7c56)c4ccccc34)cc21]

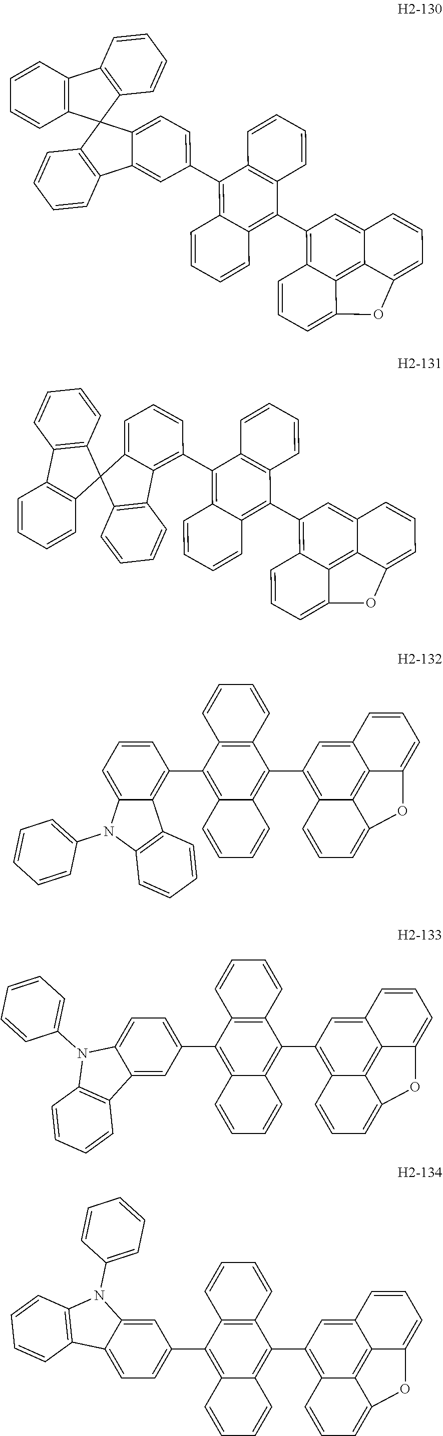 [SMILES: c1ccc2c(c1)-c1ccccc1C21c2ccccc2-c2cc(-c3c4ccccc4c(-c4cc5cccc6oc7cccc4c7c56)c4ccccc34)ccc21 ; c1ccc2c(c1)-c1ccccc1C21c2ccccc2-c2c(-c3c4ccccc4c(-c4cc5cccc6oc7cccc4c7c56)c4ccccc34)cccc21 ; c1ccc(-n2c3ccccc3c3c(-c4c5ccccc5c(-c5cc6cccc7oc8cccc5c8c67)c5ccccc45)cccc32)cc1 ; c1ccc(-n2c3ccccc3c3cc(-c4c5ccccc5c(-c5cc6cccc7oc8cccc5c8c67)c5ccccc45)ccc32)cc1 ; c1ccc(-n2c3ccccc3c3ccc(-c4c5ccccc5c(-c5cc6cccc7oc8cccc5c8c67)c5ccccc45)cc32)cc1]

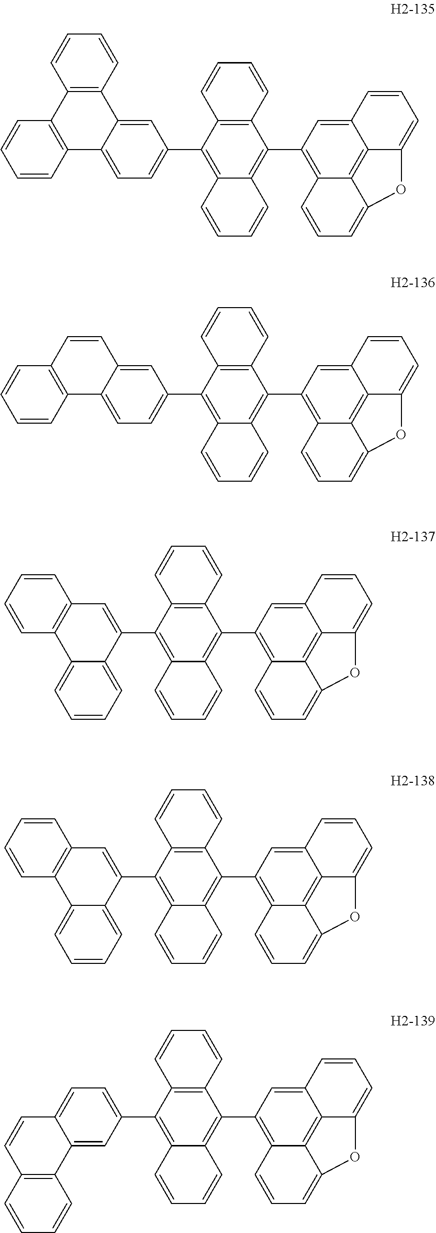 [SMILES: c1cc2cc(-c3c4ccccc4c(-c4ccc5c6ccccc6c6ccccc6c5c4)c4ccccc34)c3cccc4oc(c1)c2c43 ; c1ccc2c(c1)ccc1cc(-c3c4ccccc4c(-c4cc5cccc6oc7cccc4c7c56)c4ccccc34)ccc12 ; c1ccc2c(c1)cc(-c1c3ccccc3c(-c3cc4cccc5oc6cccc3c6c45)c3ccccc13)c1ccccc12 ; c1ccc2c(c1)cc(-c1c3ccccc3c(-c3cc4cccc5oc6cccc3c6c45)c3ccccc13)c1ccccc12 ; c1ccc2c(c1)ccc1ccc(-c3c4ccccc4c(-c4cc5cccc6oc7cccc4c7c56)c4ccccc34)cc12]

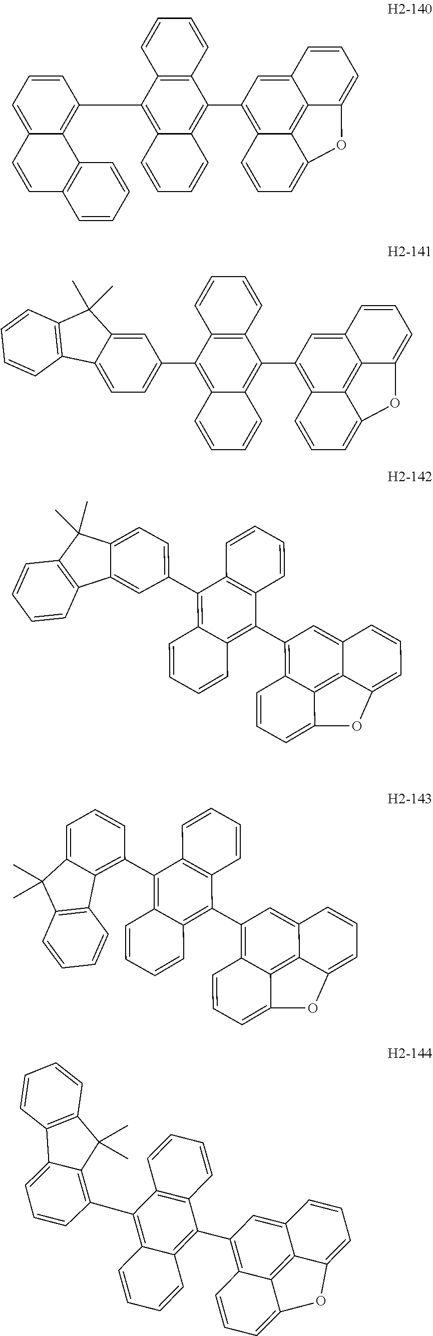 [SMILES: c1ccc2c(c1)ccc1cccc(-c3c4ccccc4c(-c4cc5cccc6oc7cccc4c7c56)c4ccccc34)c12 ; CC1(C)c2ccccc2-c2ccc(-c3c4ccccc4c(-c4cc5cccc6oc7cccc4c7c56)c4ccccc34)cc21 ; CC1(C)c2ccccc2-c2cc(-c3c4ccccc4c(-c4cc5cccc6oc7cccc4c7c56)c4ccccc34)ccc21 ; CC1(C)c2ccccc2-c2c(-c3c4ccccc4c(-c4cc5cccc6oc7cccc4c7c56)c4ccccc34)cccc21 ; CC1(C)c2ccccc2-c2cccc(-c3c4ccccc4c(-c4cc5cccc6oc7cccc4c7c56)c4ccccc34)c21]

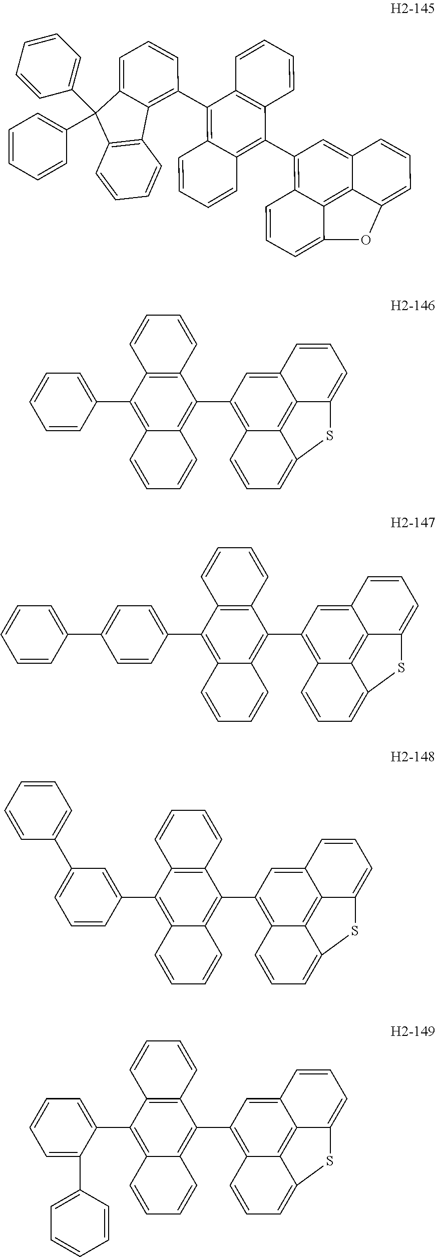 [SMILES: c1ccc(C2(c3ccccc3)c3ccccc3-c3c(-c4c5ccccc5c(-c5cc6cccc7oc8cccc5c8c67)c5ccccc45)cccc32)cc1 ; c1ccc(-c2c3ccccc3c(-c3cc4cccc5sc6cccc3c6c45)c3ccccc23)cc1 ; c1ccc(-c2ccc(-c3c4ccccc4c(-c4cc5cccc6sc7cccc4c7c56)c4ccccc34)cc2)cc1 ; c1ccc(-c2cccc(-c3c4ccccc4c(-c4cc5cccc6sc7cccc4c7c56)c4ccccc34)c2)cc1 ; c1ccc(-c2ccccc2-c2c3ccccc3c(-c3cc4cccc5sc6cccc3c6c45)c3ccccc23)cc1]

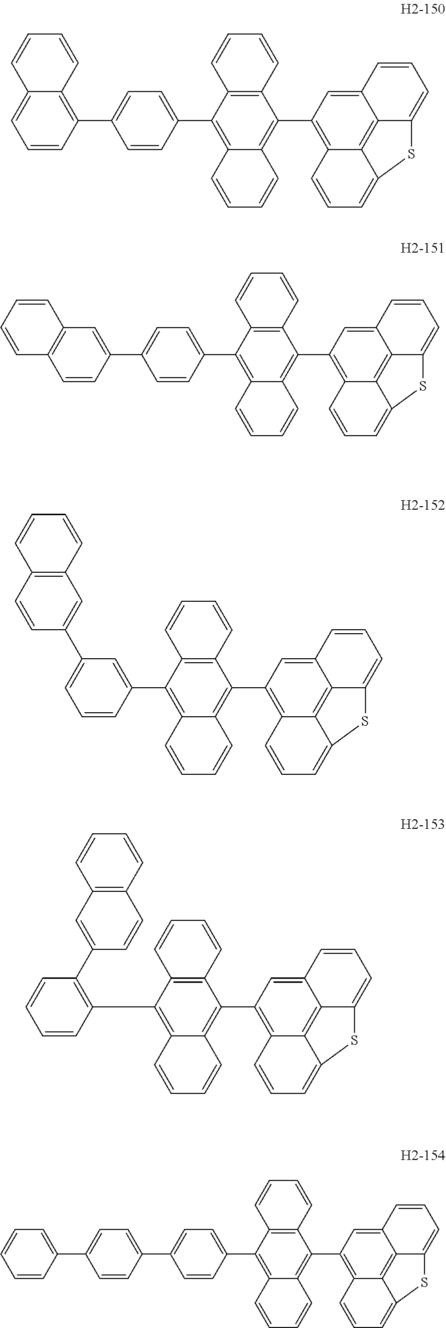 [SMILES: c1ccc2c(-c3ccc(-c4c5ccccc5c(-c5cc6cccc7sc8cccc5c8c67)c5ccccc45)cc3)cccc2c1 ; c1ccc2cc(-c3ccc(-c4c5ccccc5c(-c5cc6cccc7sc8cccc5c8c67)c5ccccc45)cc3)ccc2c1 ; c1cc(-c2ccc3ccccc3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3ccccc3c2)c1 ; c1ccc(-c2ccc(-c3ccc(-c4c5ccccc5c(-c5cc6cccc7sc8cccc5c8c67)c5ccccc45)cc3)cc2)cc1]

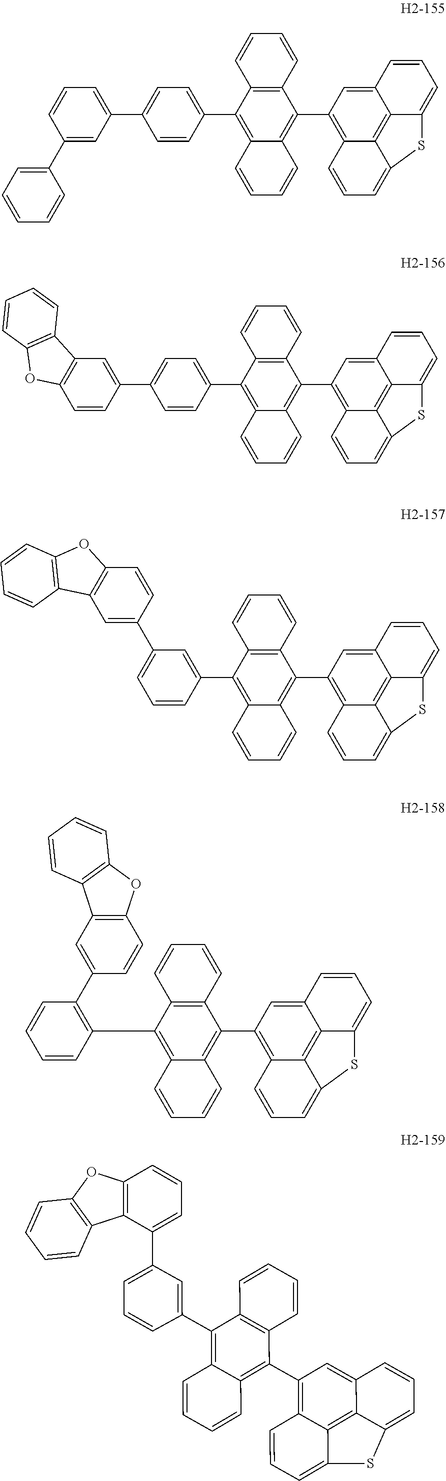 [SMILES: c1ccc(-c2cccc(-c3ccc(-c4c5ccccc5c(-c5cc6cccc7sc8cccc5c8c67)c5ccccc45)cc3)c2)cc1 ; c1ccc2c(c1)oc1ccc(-c3ccc(-c4c5ccccc5c(-c5cc6cccc7sc8cccc5c8c67)c5ccccc45)cc3)cc12 ; c1cc(-c2ccc3oc4ccccc4c3c2)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3oc4ccccc4c3c2)c1 ; c1cc(-c2c3ccccc3c(-c3cc4cccc5sc6cccc3c6c45)c3ccccc23)cc(-c2cccc3oc4ccccc4c23)c1]

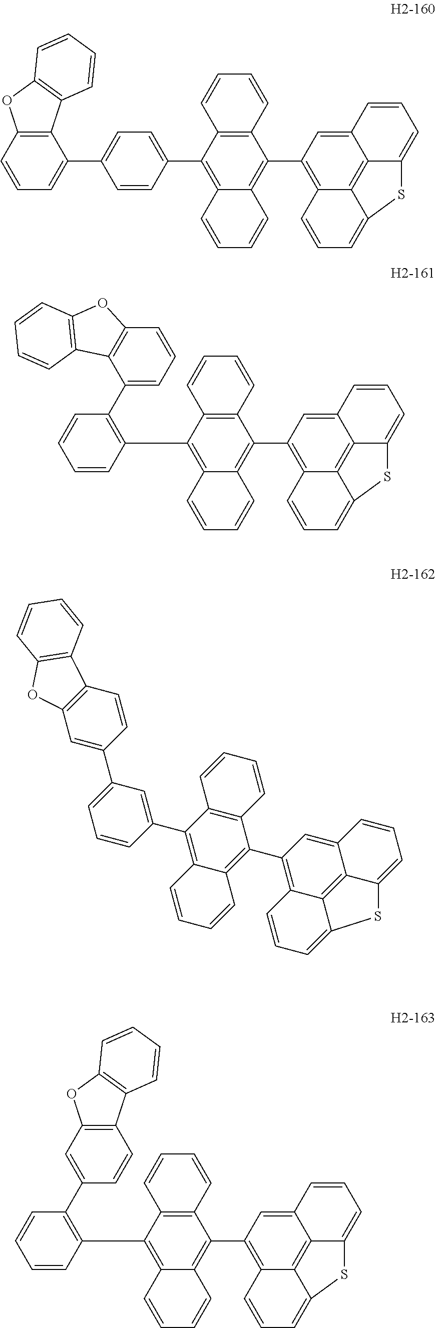 [SMILES: c1ccc2c(c1)oc1cccc(-c3ccc(-c4c5ccccc5c(-c5cc6cccc7sc8cccc5c8c67)c5ccccc45)cc3)c12 ; c1ccc(-c2cccc3oc4ccccc4c23)c(-c2c3ccccc3c(-c3cc4cccc5sc6cccc3c6c45)c3ccccc23)c1 ; c1cc(-c2ccc3c(c2)oc2ccccc23)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3c(c2)oc2ccccc23)c1]

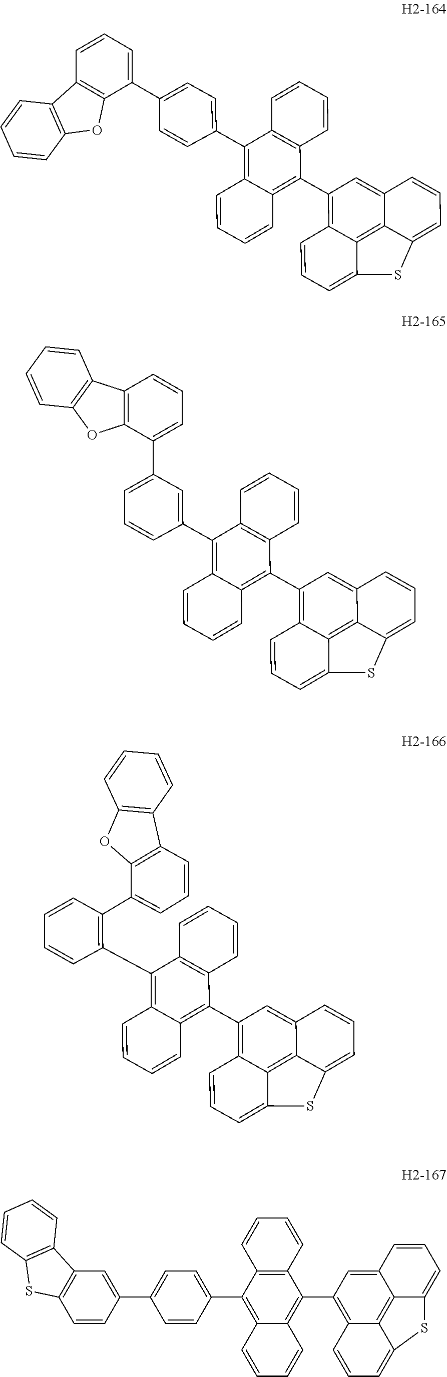 [SMILES: c1ccc2c(c1)oc1c(-c3ccc(-c4c5ccccc5c(-c5cc6cccc7sc8cccc5c8c67)c5ccccc45)cc3)cccc12 ; c1cc(-c2c3ccccc3c(-c3cc4cccc5sc6cccc3c6c45)c3ccccc23)cc(-c2cccc3c2oc2ccccc23)c1 ; c1ccc(-c2cccc3c2oc2ccccc23)c(-c2c3ccccc3c(-c3cc4cccc5sc6cccc3c6c45)c3ccccc23)c1 ; c1ccc2c(c1)sc1ccc(-c3ccc(-c4c5ccccc5c(-c5cc6cccc7sc8cccc5c8c67)c5ccccc45)cc3)cc12]

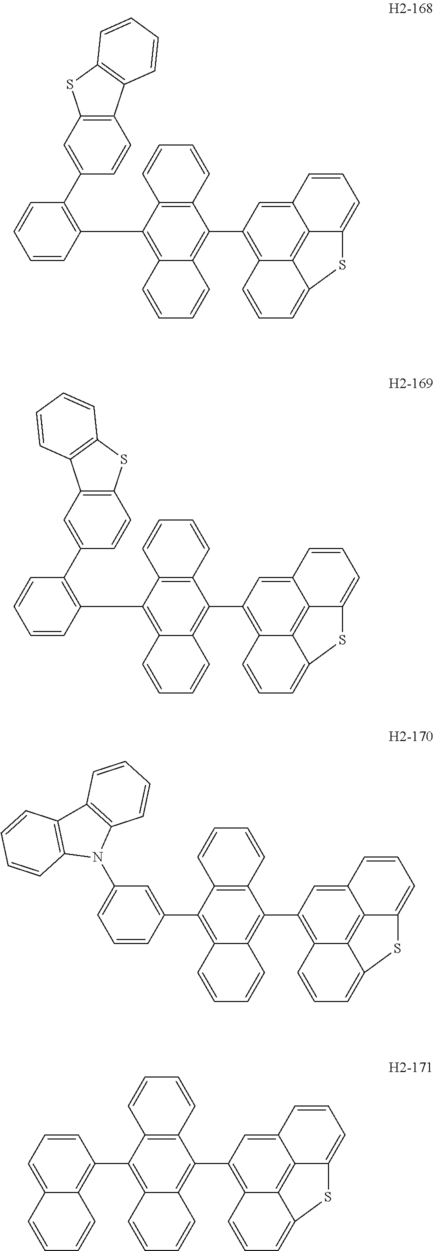 [SMILES: c1ccc(-c2c3ccccc3c(-c3cc4cccc5sc6cccc3c6c45)c3ccccc23)c(-c2ccc3c(c2)sc2ccccc23)c1 ; c1ccc(-c2c3ccccc3c(-c3cc4cccc5sc6cccc3c6c45)c3ccccc23)c(-c2ccc3sc4ccccc4c3c2)c1 ; c1cc(-c2c3ccccc3c(-c3cc4cccc5sc6cccc3c6c45)c3ccccc23)cc(-n2c3ccccc3c3ccccc32)c1 ; c1ccc2c(-c3c4ccccc4c(-c4cc5cccc6sc7cccc4c7c56)c4ccccc34)cccc2c1]

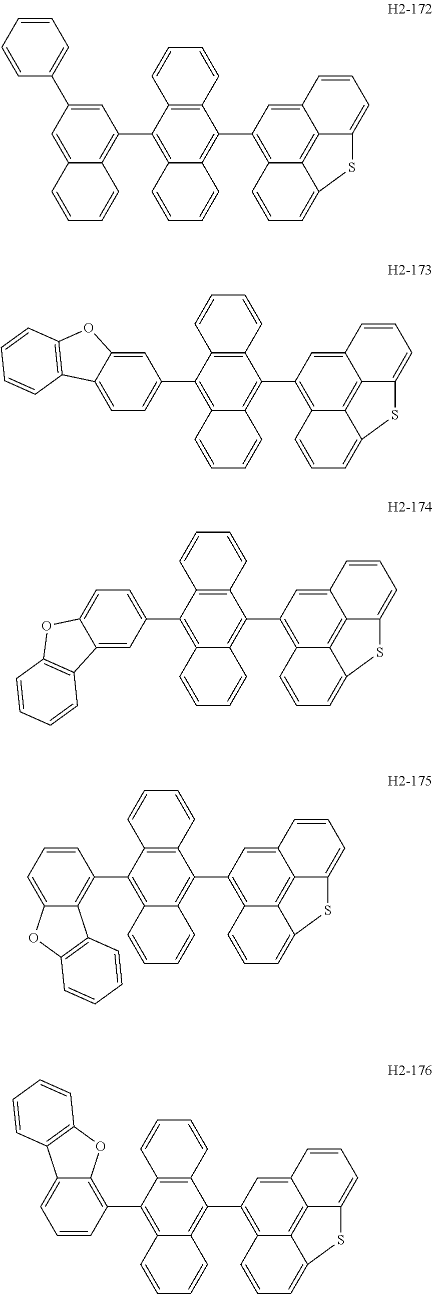

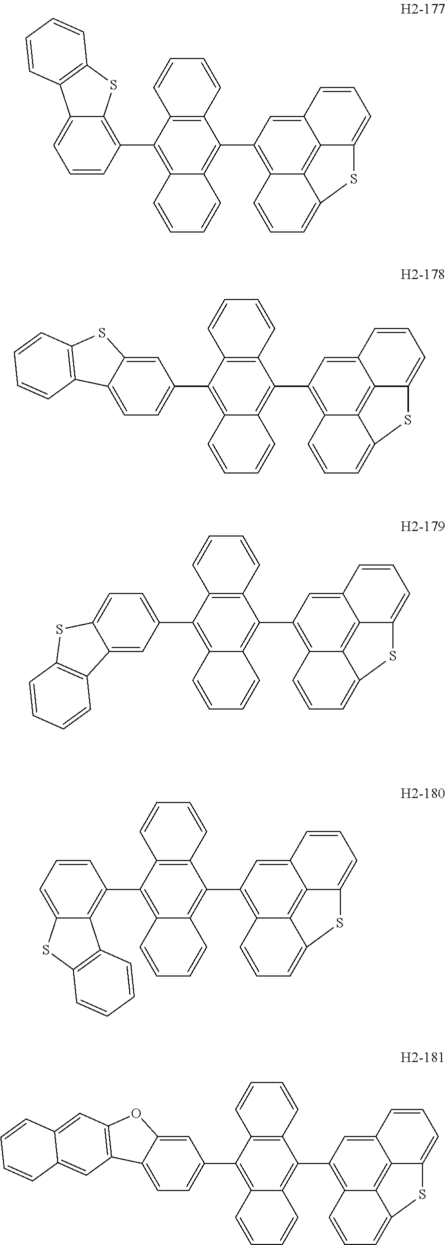 [SMILES: c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; c1ccc2cc3c(cc2c1)oc1cc(-c2c4ccccc4c(-c4cc5cccc6sc7cccc4c7c56)c4ccccc24)ccc13]

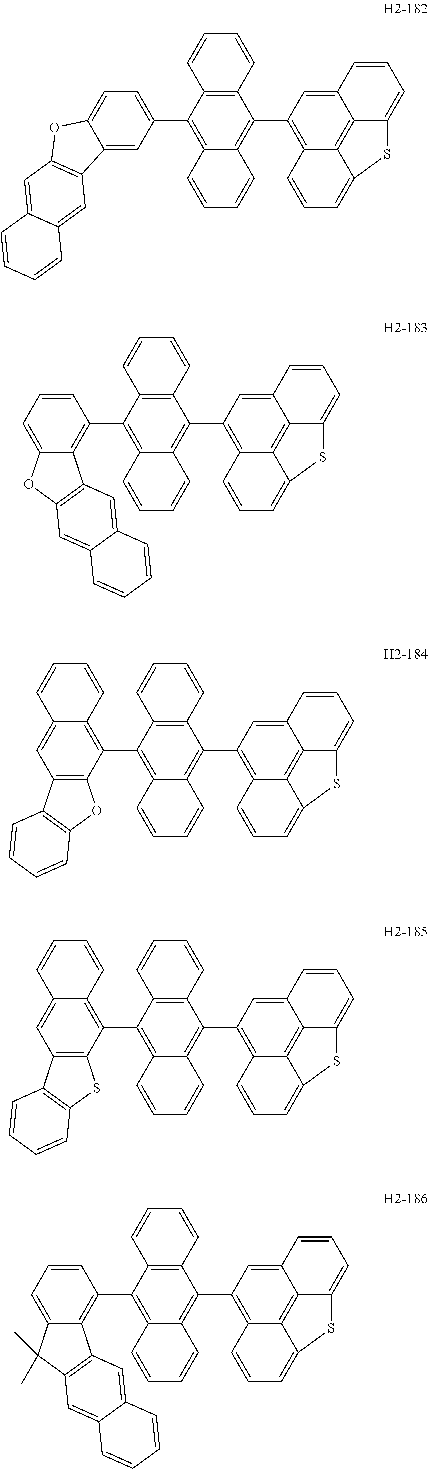 [SMILES: c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1 ; c1ccc2c(-c3c4ccccc4c(-c4cc5cccc6sc7cccc4c7c56)c4ccccc34)c3sc4ccccc4c3cc2c1 ; CC1(C)c2cc3ccccc3cc2-c2c(-c3c4ccccc4c(-c4cc5cccc6sc7cccc4c7c56)c4ccccc34)cccc21]

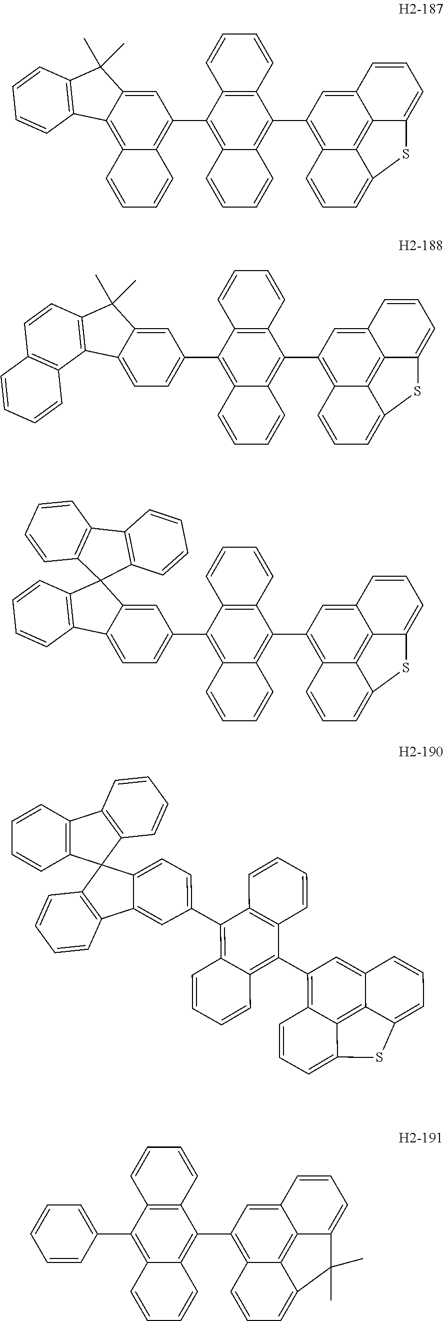

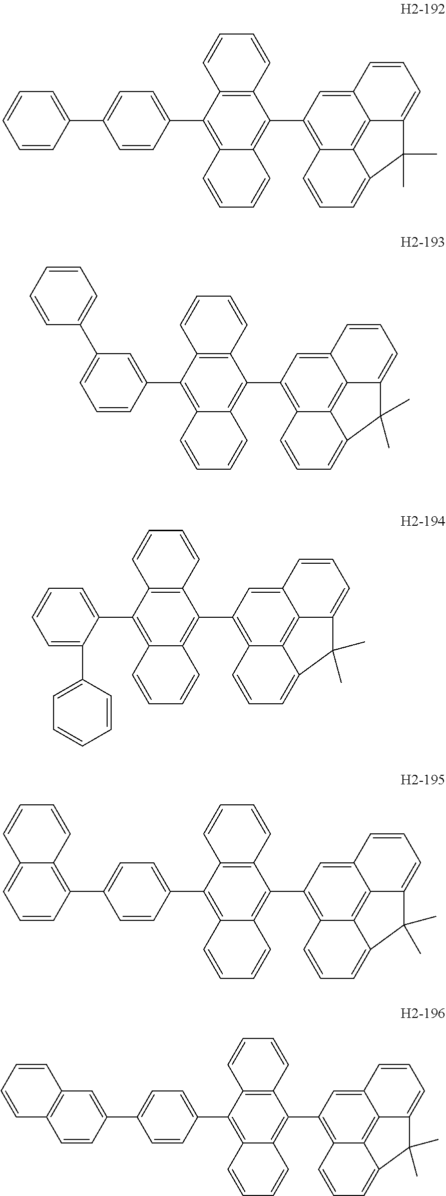

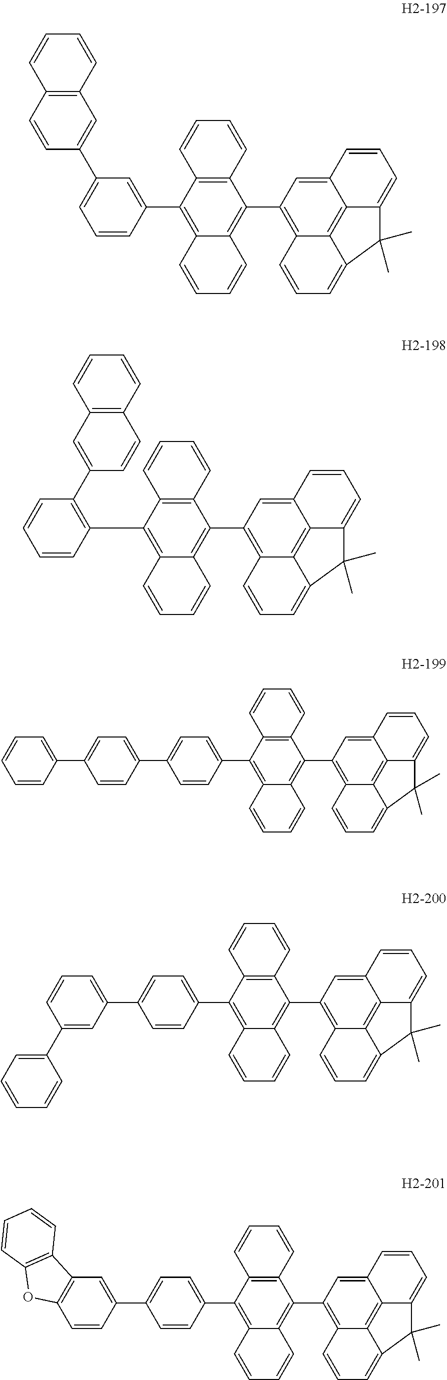

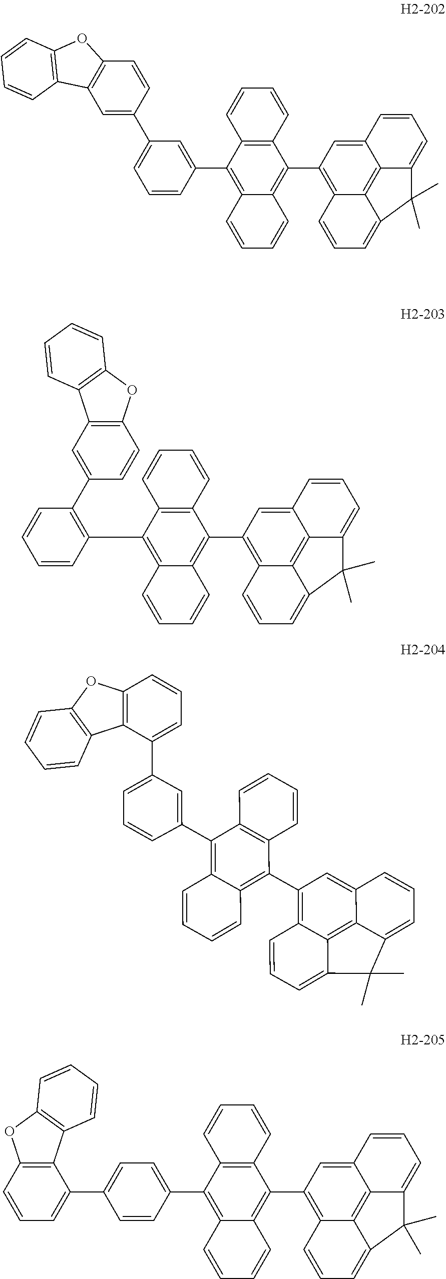 [SMILES: CC1(C)c2cccc3cc(-c4c5ccccc5c(-c5cccc(-c6ccc7oc8ccccc8c7c6)c5)c5ccccc45)c4cccc1c4c23 ; CC1(C)c2cccc3cc(-c4c5ccccc5c(-c5ccccc5-c5ccc6oc7ccccc7c6c5)c5ccccc45)c4cccc1c4c23 ; CC1(C)c2cccc3cc(-c4c5ccccc5c(-c5cccc(-c6cccc7oc8ccccc8c67)c5)c5ccccc45)c4cccc1c4c23 ; CC1(C)c2cccc3cc(-c4c5ccccc5c(-c5ccc(-c6cccc7oc8ccccc8c67)cc5)c5ccccc45)c4cccc1c4c23]

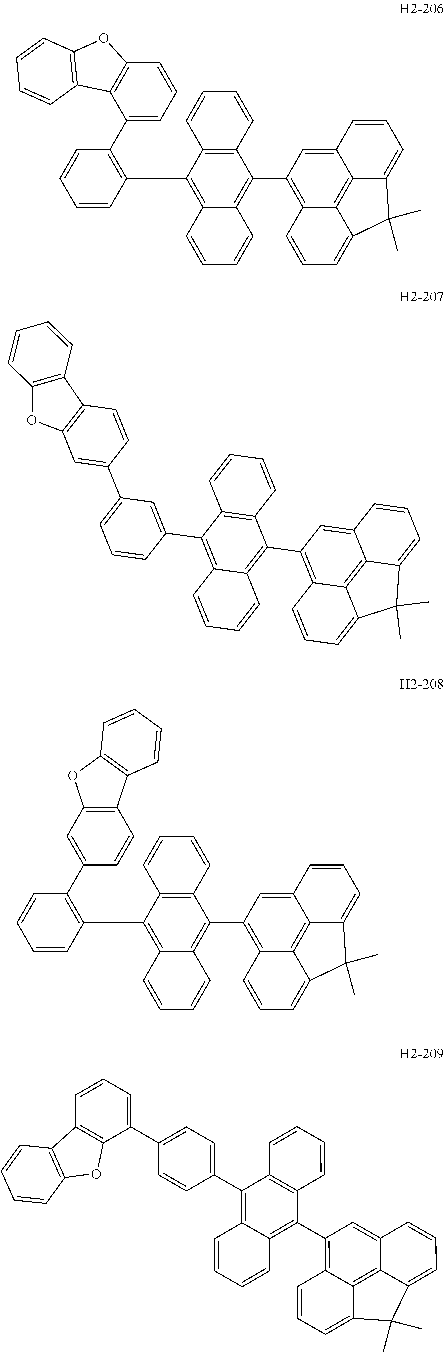 [SMILES: CC1(C)c2cccc3cc(-c4c5ccccc5c(-c5ccccc5-c5cccc6oc7ccccc7c56)c5ccccc45)c4cccc1c4c23 ; CC1(C)c2cccc3cc(-c4c5ccccc5c(-c5cccc(-c6ccc7c(c6)oc6ccccc67)c5)c5ccccc45)c4cccc1c4c23 ; CC1(C)c2cccc3cc(-c4c5ccccc5c(-c5ccccc5-c5ccc6c(c5)oc5ccccc56)c5ccccc45)c4cccc1c4c23 ; CC1(C)c2cccc3cc(-c4c5ccccc5c(-c5ccc(-c6cccc7c6oc6ccccc67)cc5)c5ccccc45)c4cccc1c4c23]

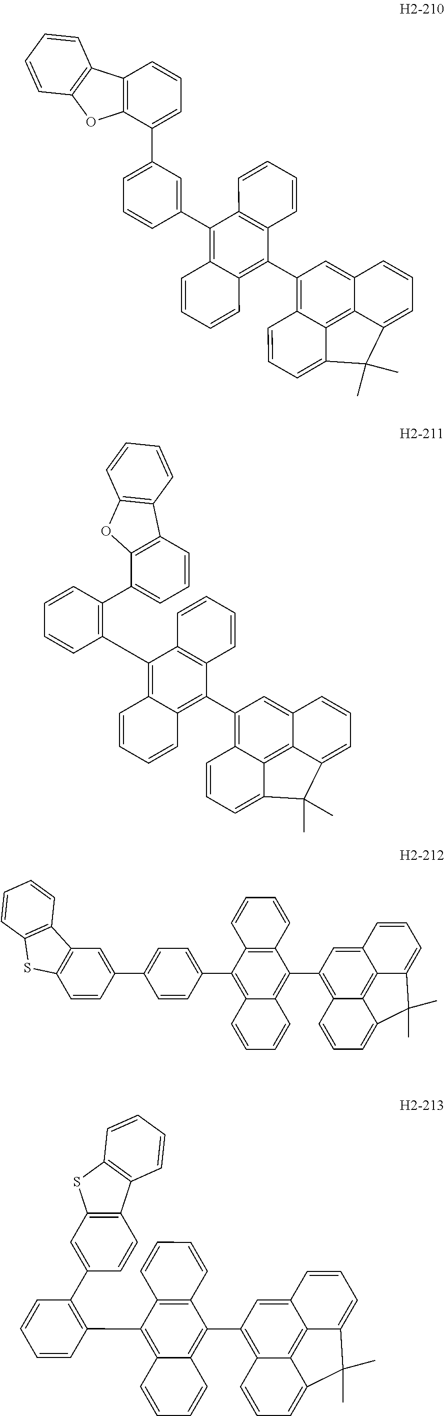 [SMILES: CC1(C)c2cccc3cc(-c4c5ccccc5c(-c5cccc(-c6cccc7c6oc6ccccc67)c5)c5ccccc45)c4cccc1c4c23 ; CC1(C)c2cccc3cc(-c4c5ccccc5c(-c5ccccc5-c5cccc6c5oc5ccccc56)c5ccccc45)c4cccc1c4c23 ; CC1(C)c2cccc3cc(-c4c5ccccc5c(-c5ccc(-c6ccc7sc8ccccc8c7c6)cc5)c5ccccc45)c4cccc1c4c23 ; CC1(C)c2cccc3cc(-c4c5ccccc5c(-c5ccccc5-c5ccc6c(c5)sc5ccccc56)c5ccccc45)c4cccc1c4c23]

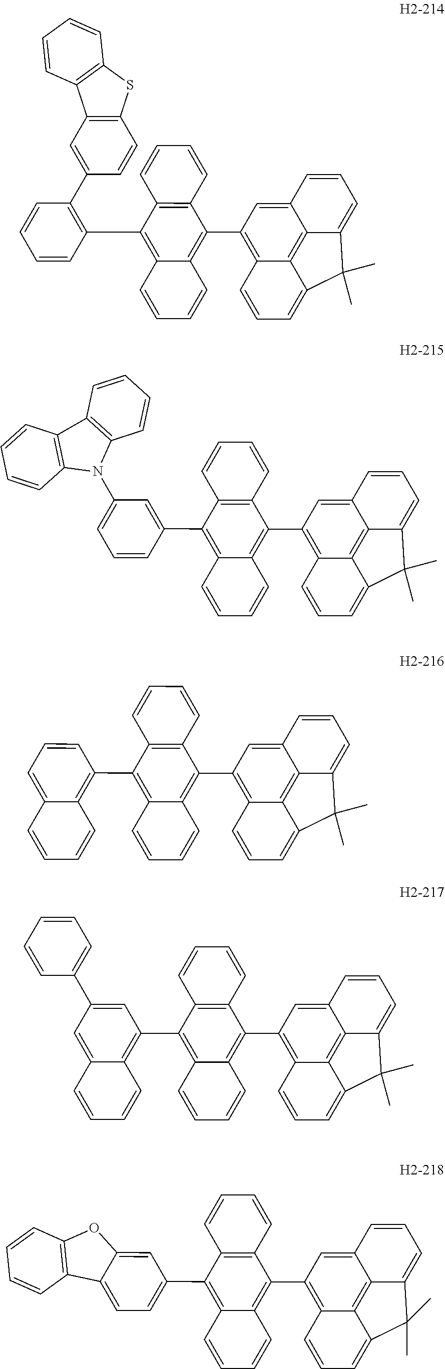

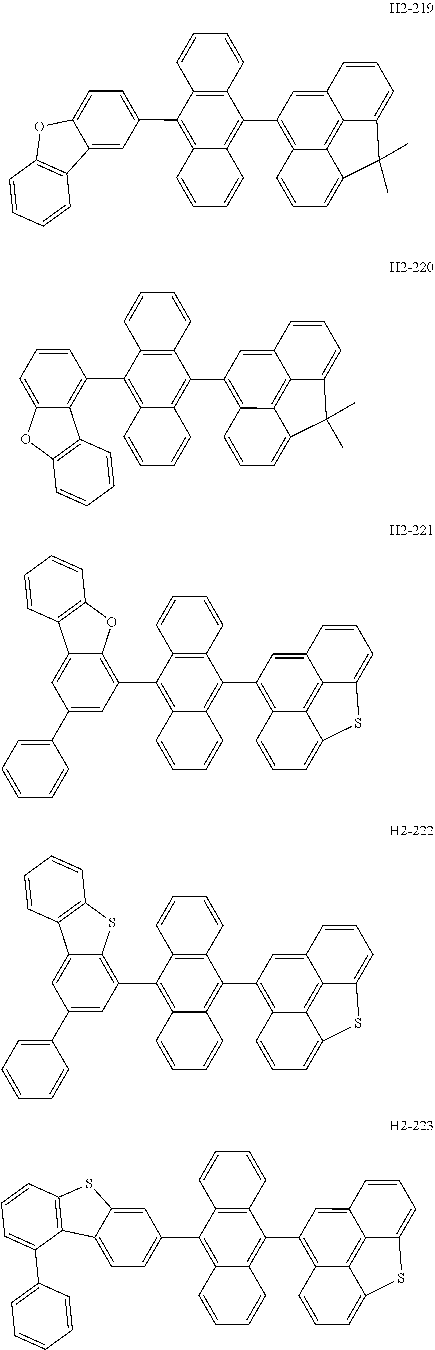 [SMILES: CC1(C)c2cccc3cc(-c4c5ccccc5c(-c5ccc6oc7ccccc7c6c5)c5ccccc45)c4cccc1c4c23 ; CC1(C)c2cccc3cc(-c4c5ccccc5c(-c5cccc6oc7ccccc7c56)c5ccccc45)c4cccc1c4c23 ; c1ccc(-c2cc(-c3c4ccccc4c(-c4cc5cccc6sc7cccc4c7c56)c4ccccc34)c3oc4ccccc4c3c2)cc1 ; c1ccc(-c2cc(-c3c4ccccc4c(-c4cc5cccc6sc7cccc4c7c56)c4ccccc34)c3sc4ccccc4c3c2)cc1 ; c1ccc(-c2cccc3sc4cc(-c5c6ccccc6c(-c6cc7cccc8sc9cccc6c9c78)c6ccccc56)ccc4c23)cc1]

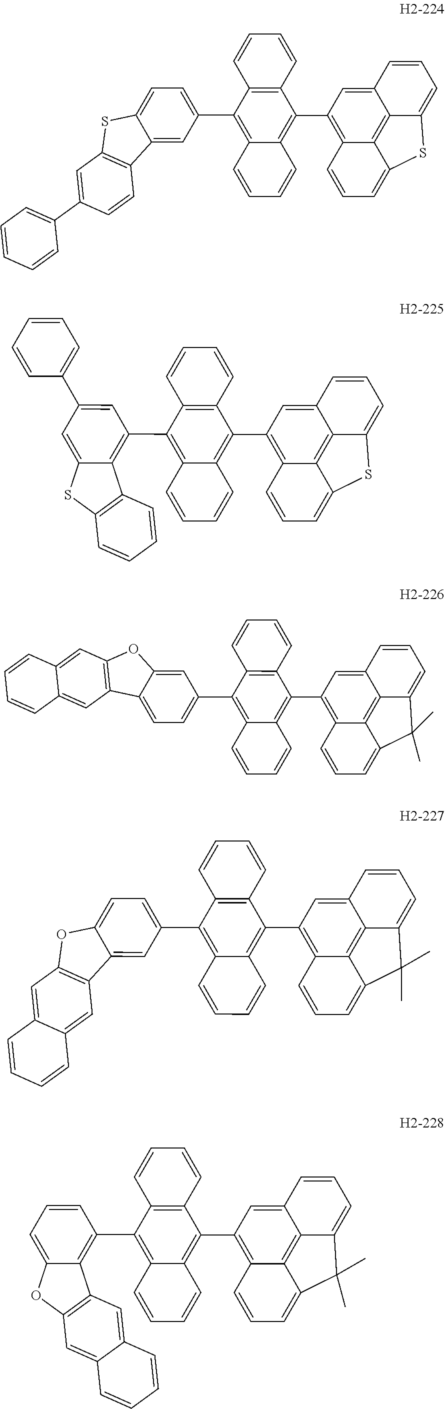 [SMILES: c1ccc(-c2ccc3c(c2)sc2ccc(-c4c5ccccc5c(-c5cc6cccc7sc8cccc5c8c67)c5ccccc45)cc23)cc1 ; c1ccc(-c2cc(-c3c4ccccc4c(-c4cc5cccc6sc7cccc4c7c56)c4ccccc34)c3c(c2)sc2ccccc23)cc1 ; CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23]

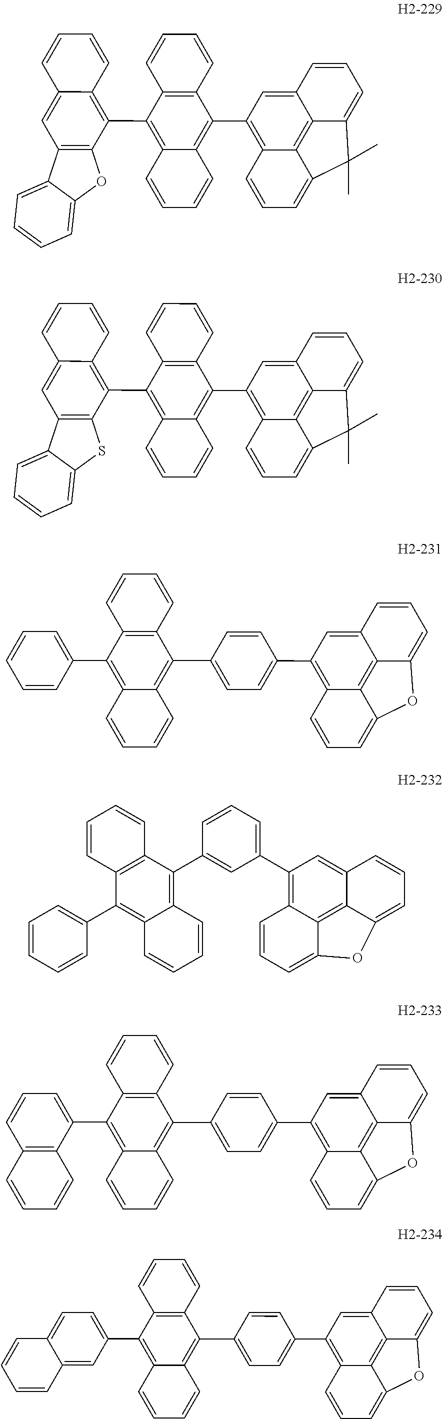 [SMILES: CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23 ; c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1 ; c1ccc2cc(-c3c4ccccc4c(-c4ccc(-c5cc6cccc7oc8cccc5c8c67)cc4)c4ccccc34)ccc2c1]

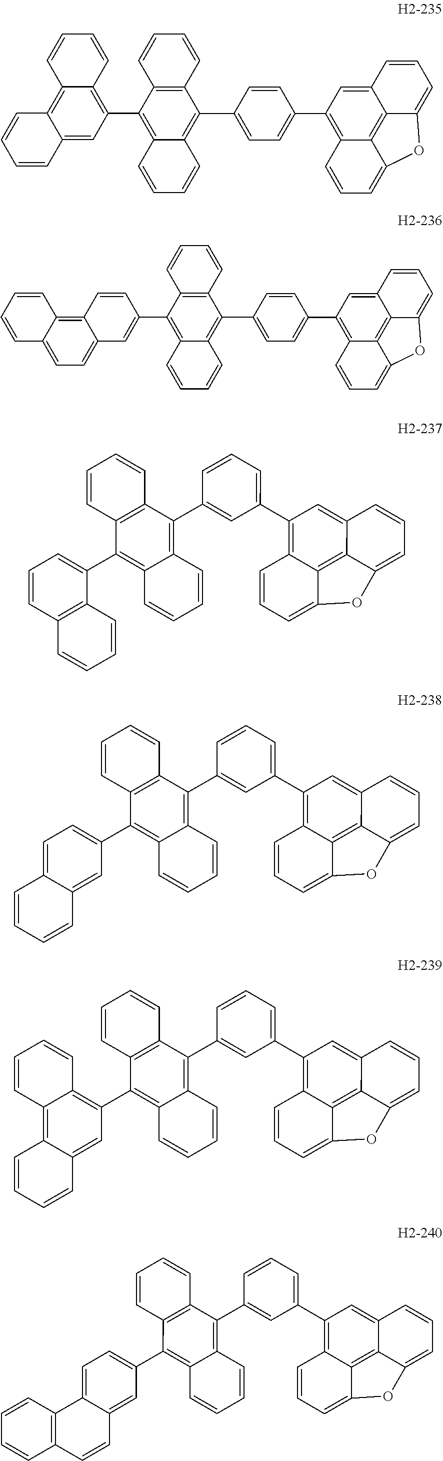 [SMILES: c1ccc2c(c1)cc(-c1c3ccccc3c(-c3ccc(-c4cc5cccc6oc7cccc4c7c56)cc3)c3ccccc13)c1ccccc12 ; c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1 ; c1cc(-c2c3ccccc3c(-c3ccc4c(ccc5ccccc54)c3)c3ccccc23)cc(-c2cc3cccc4oc5cccc2c5c34)c1]

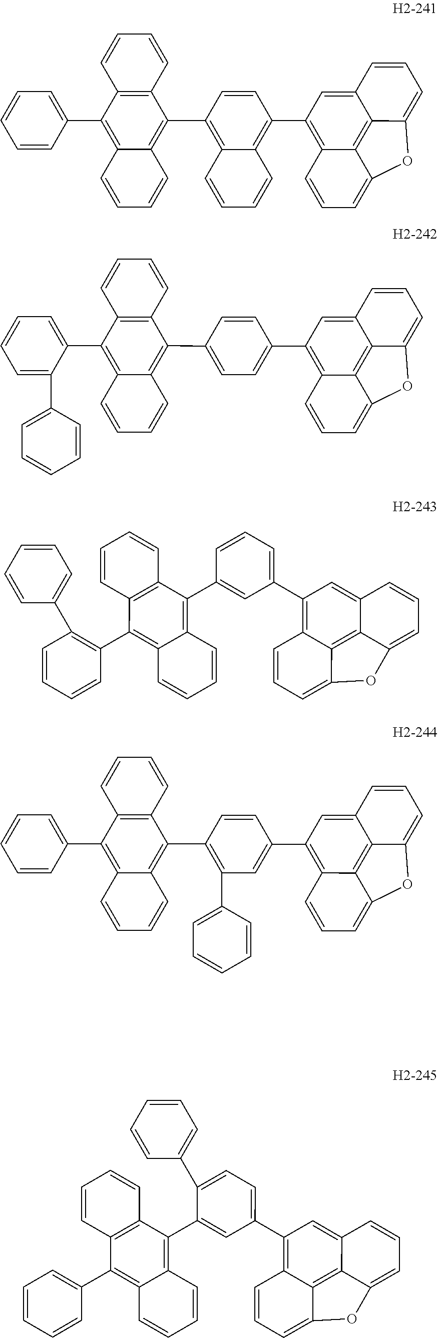 [SMILES: c1ccc(-c2c3ccccc3c(-c3ccc(-c4cc5cccc6oc7cccc4c7c56)c4ccccc34)c3ccccc23)cc1 ; c1ccc(-c2ccccc2-c2c3ccccc3c(-c3ccc(-c4cc5cccc6oc7cccc4c7c56)cc3)c3ccccc23)cc1 ; c1ccc(-c2ccccc2-c2c3ccccc3c(-c3cccc(-c4cc5cccc6oc7cccc4c7c56)c3)c3ccccc23)cc1 ; c1ccc(-c2cc(-c3cc4cccc5oc6cccc3c6c45)ccc2-c2c3ccccc3c(-c3ccccc3)c3ccccc23)cc1 ; c1ccc(-c2ccc(-c3cc4cccc5oc6cccc3c6c45)cc2-c2c3ccccc3c(-c3ccccc3)c3ccccc23)cc1]

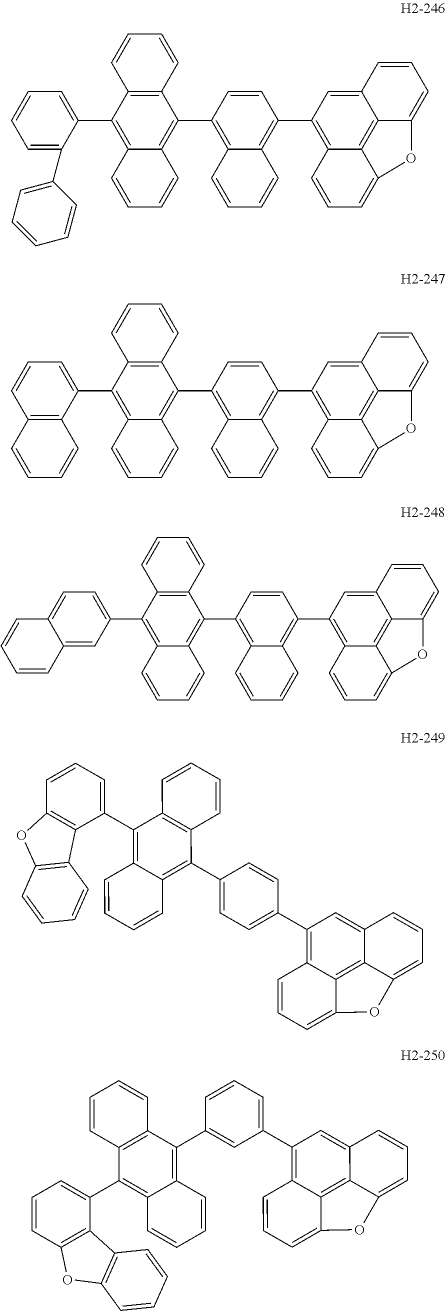 [SMILES: c1ccc(-c2ccccc2-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1]

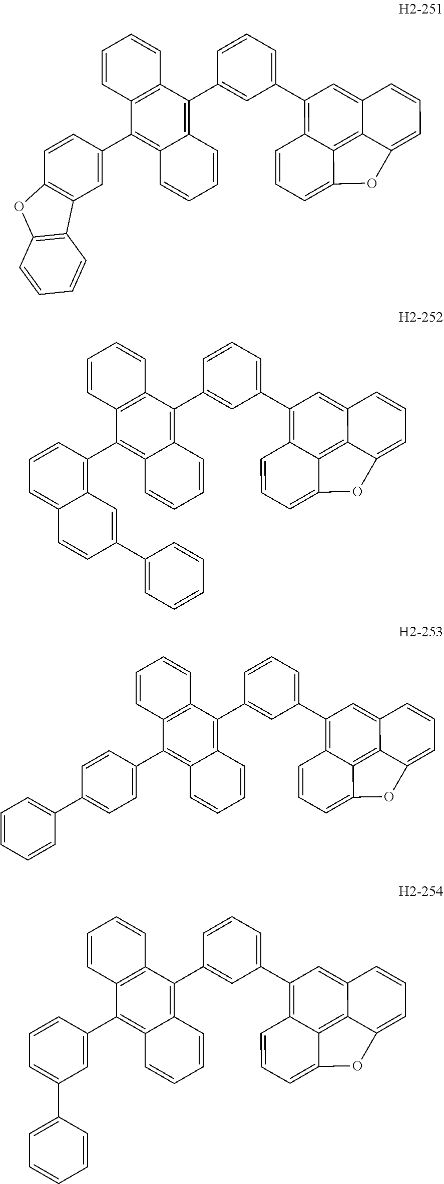 [SMILES: c1cc(-c2c3ccccc3c(-c3ccc4oc5ccccc5c4c3)c3ccccc23)cc(-c2cc3cccc4oc5cccc2c5c34)c1 ; c1ccc(-c2ccc3cccc(-c4c5ccccc5c(-c5cccc(-c6cc7cccc8oc9cccc6c9c78)c5)c5ccccc45)c3c2)cc1 ; c1ccc(-c2ccc(-c3c4ccccc4c(-c4cccc(-c5cc6cccc7oc8cccc5c8c67)c4)c4ccccc34)cc2)cc1 ; c1ccc(-c2cccc(-c3c4ccccc4c(-c4cccc(-c5cc6cccc7oc8cccc5c8c67)c4)c4ccccc34)c2)cc1]

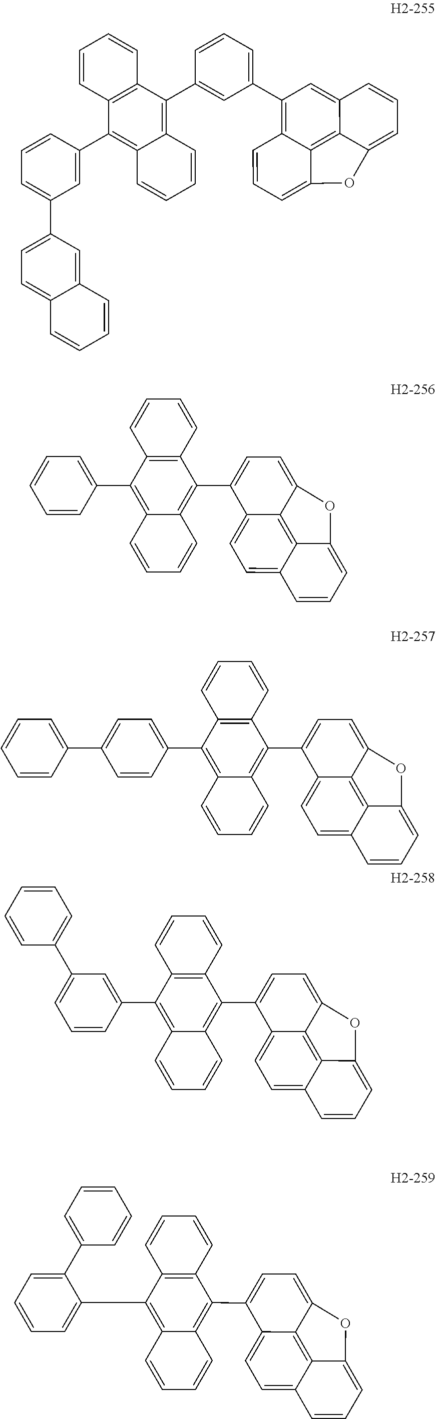 [SMILES: c1cc(-c2ccc3ccccc3c2)cc(-c2c3ccccc3c(-c3cccc(-c4cc5cccc6oc7cccc4c7c56)c3)c3ccccc23)c1 ; c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc(-c2ccccc2-c2c3ccccc3c(-c3ccc4oc5cccc6ccc3c4c65)c3ccccc23)cc1]

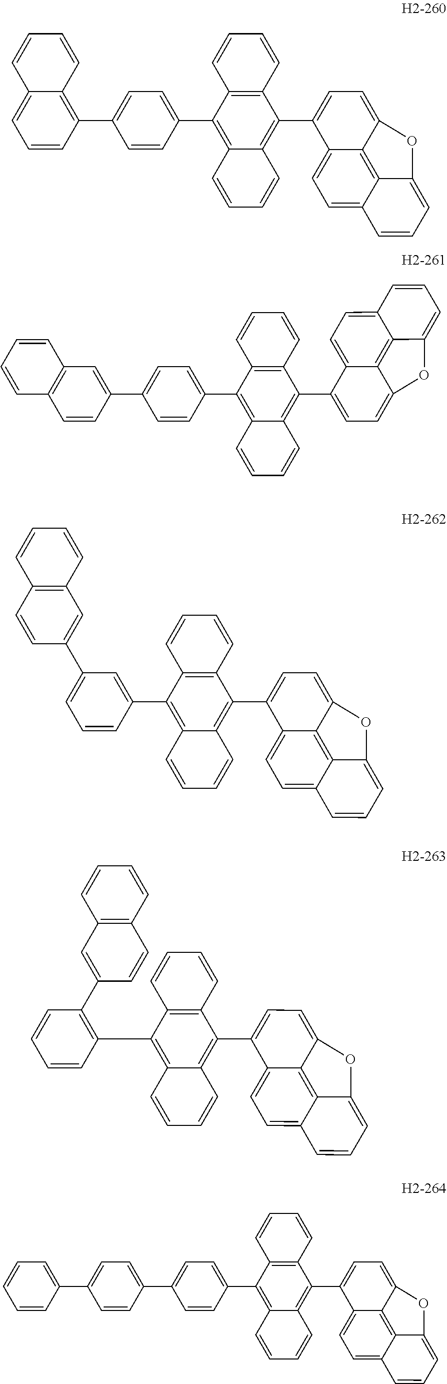 [SMILES: c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1 ; c1cc(-c2ccc3ccccc3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccccc3c2)c1 ; c1ccc(-c2ccc(-c3ccc(-c4c5ccccc5c(-c5ccc6oc7cccc8ccc5c6c87)c5ccccc45)cc3)cc2)cc1]

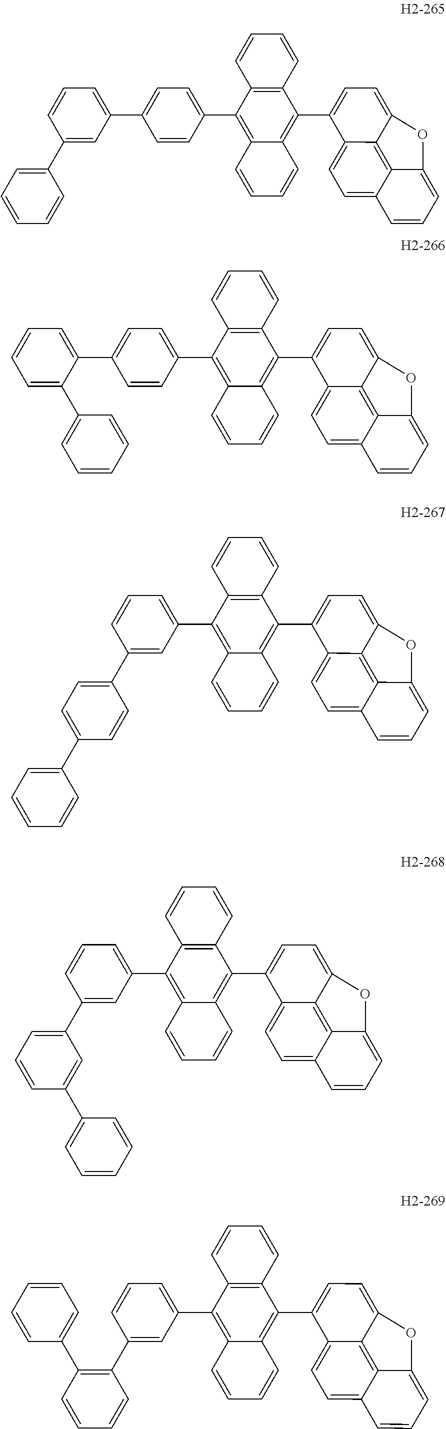 [SMILES: c1ccc(-c2cccc(-c3ccc(-c4c5ccccc5c(-c5ccc6oc7cccc8ccc5c6c87)c5ccccc45)cc3)c2)cc1 ; c1ccc(-c2ccccc2-c2ccc(-c3c4ccccc4c(-c4ccc5oc6cccc7ccc4c5c76)c4ccccc34)cc2)cc1 ; c1ccc(-c2ccc(-c3cccc(-c4c5ccccc5c(-c5ccc6oc7cccc8ccc5c6c87)c5ccccc45)c3)cc2)cc1 ; c1ccc(-c2cccc(-c3cccc(-c4c5ccccc5c(-c5ccc6oc7cccc8ccc5c6c87)c5ccccc45)c3)c2)cc1 ; c1ccc(-c2ccccc2-c2cccc(-c3c4ccccc4c(-c4ccc5oc6cccc7ccc4c5c76)c4ccccc34)c2)cc1]

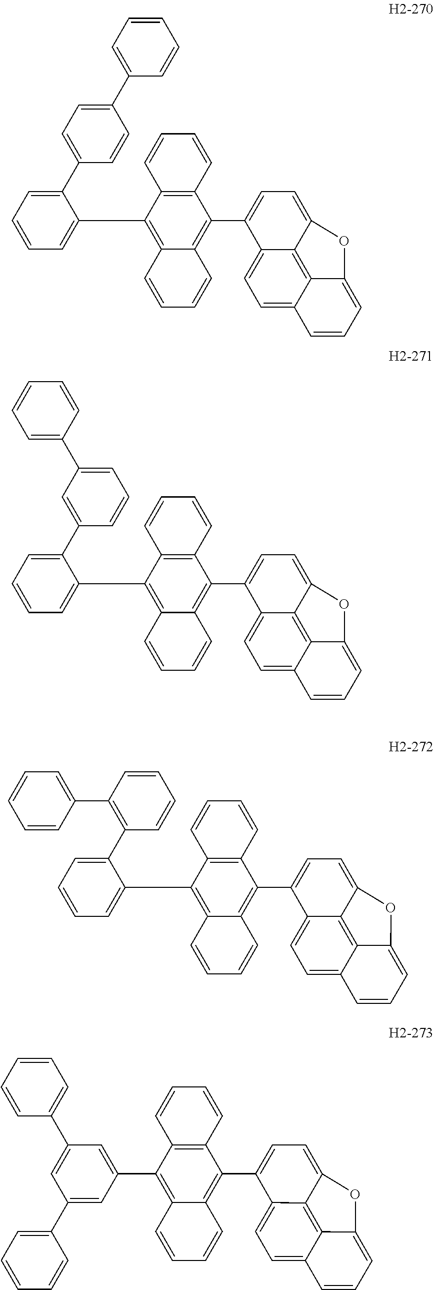 [SMILES: c1ccc(-c2ccc(-c3ccccc3-c3c4ccccc4c(-c4ccc5oc6cccc7ccc4c5c76)c4ccccc34)cc2)cc1 ; c1ccc(-c2cccc(-c3ccccc3-c3c4ccccc4c(-c4ccc5oc6cccc7ccc4c5c76)c4ccccc34)c2)cc1 ; c1ccc(-c2ccccc2-c2ccccc2-c2c3ccccc3c(-c3ccc4oc5cccc6ccc3c4c65)c3ccccc23)cc1 ; c1ccc(-c2cc(-c3ccccc3)cc(-c3c4ccccc4c(-c4ccc5oc6cccc7ccc4c5c76)c4ccccc34)c2)cc1]

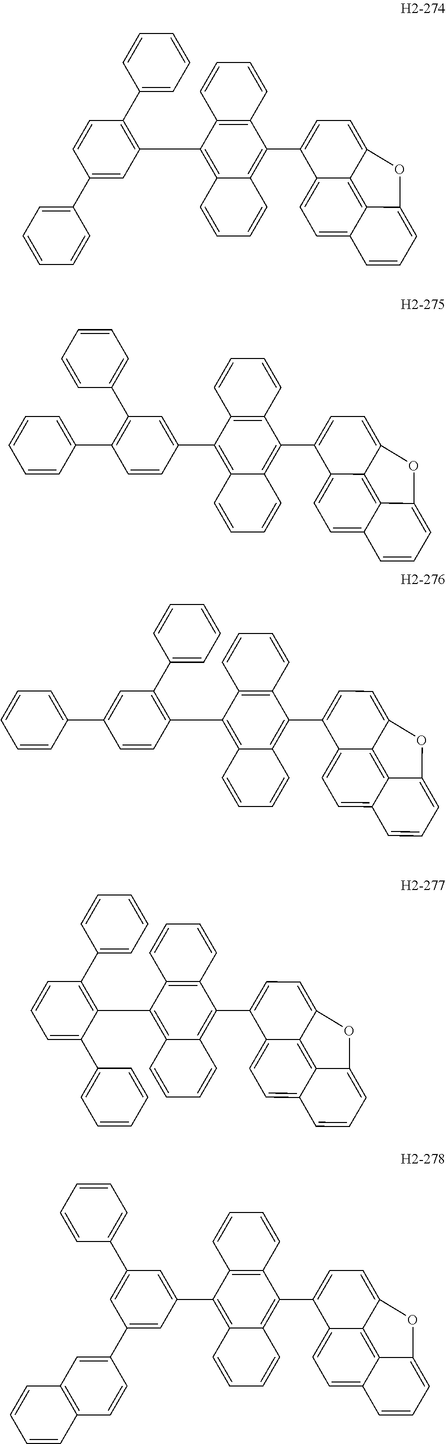 [SMILES: c1ccc(-c2ccc(-c3ccccc3)c(-c3c4ccccc4c(-c4ccc5oc6cccc7ccc4c5c76)c4ccccc34)c2)cc1 ; c1ccc(-c2ccc(-c3c4ccccc4c(-c4ccc5oc6cccc7ccc4c5c76)c4ccccc34)cc2-c2ccccc2)cc1 ; c1ccc(-c2ccc(-c3c4ccccc4c(-c4ccc5oc6cccc7ccc4c5c76)c4ccccc34)c(-c3ccccc3)c2)cc1 ; c1ccc(-c2cccc(-c3ccccc3)c2-c2c3ccccc3c(-c3ccc4oc5cccc6ccc3c4c65)c3ccccc23)cc1 ; c1ccc(-c2cc(-c3ccc4ccccc4c3)cc(-c3c4ccccc4c(-c4ccc5oc6cccc7ccc4c5c76)c4ccccc34)c2)cc1]

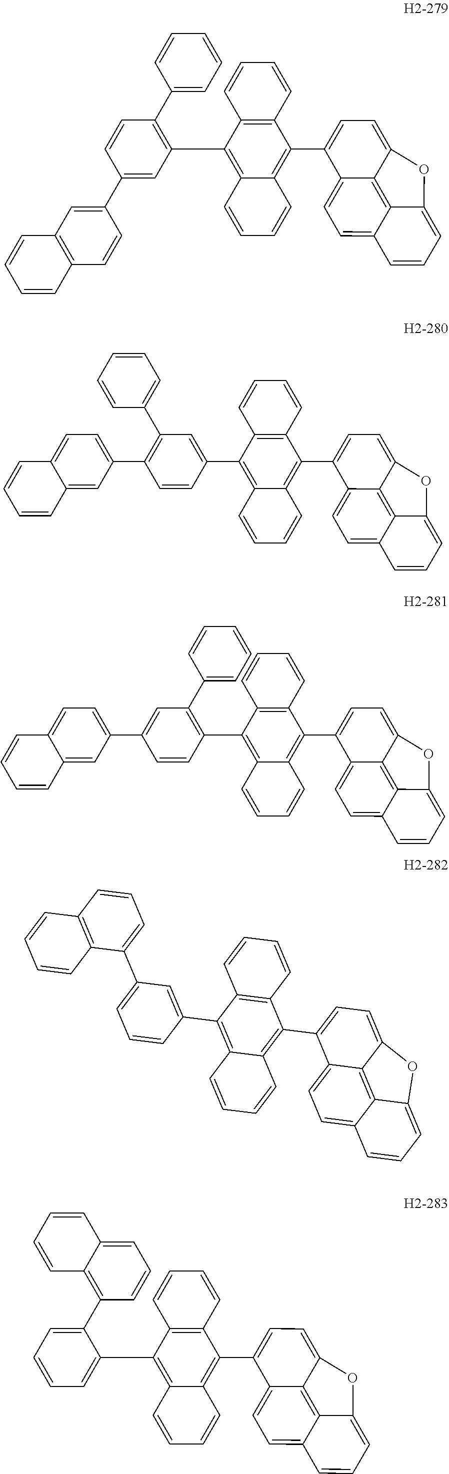 [SMILES: c1ccc(-c2ccc(-c3ccc4ccccc4c3)cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)ccc2-c2ccc3ccccc3c2)cc1 ; c1ccc(-c2cc(-c3ccc4ccccc4c3)ccc2-c2c3ccccc3c(-c3ccc4oc5cccc6ccc3c4c65)c3ccccc23)cc1 ; c1cc(-c2cccc3ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2cccc3ccccc23)c1]

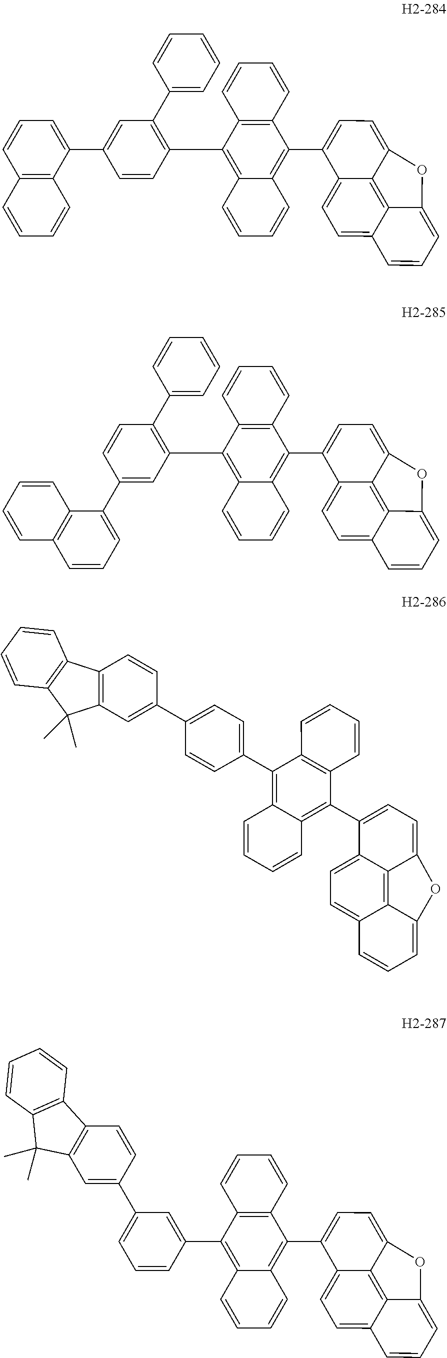 [SMILES: c1ccc(-c2cc(-c3cccc4ccccc34)ccc2-c2c3ccccc3c(-c3ccc4oc5cccc6ccc3c4c65)c3ccccc23)cc1 ; c1ccc(-c2ccc(-c3cccc4ccccc34)cc2-c2c3ccccc3c(-c3ccc4oc5cccc6ccc3c4c65)c3ccccc23)cc1 ; CC1(C)c2ccccc2-c2ccc(-c3ccc(-c4c5ccccc5c(-c5ccc6oc7cccc8ccc5c6c87)c5ccccc45)cc3)cc21 ; CC1(C)c2ccccc2-c2ccc(-c3cccc(-c4c5ccccc5c(-c5ccc6oc7cccc8ccc5c6c87)c5ccccc45)c3)cc21]

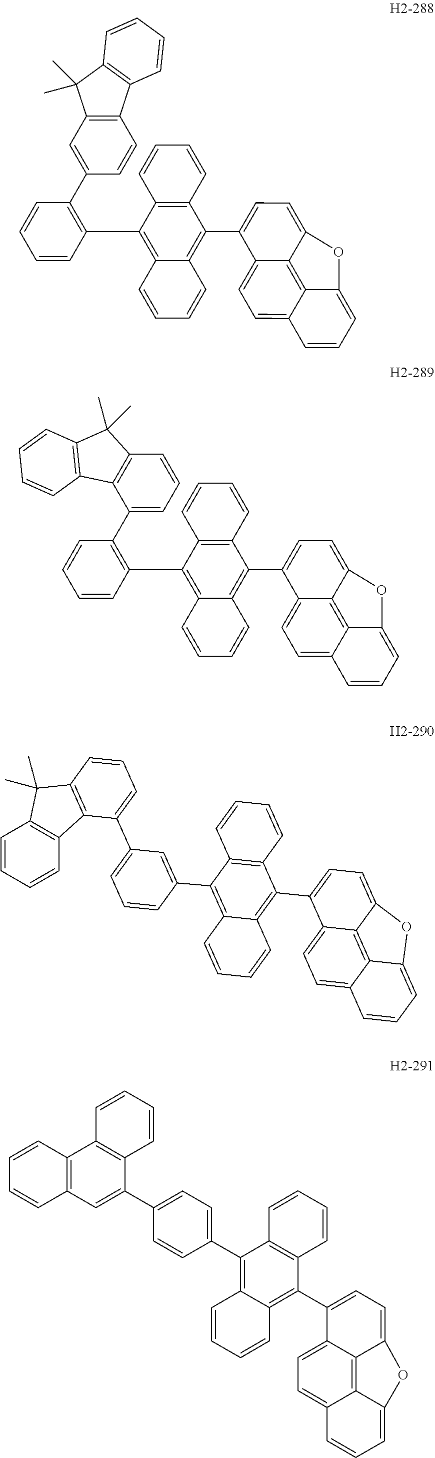 [SMILES: CC1(C)c2ccccc2-c2ccc(-c3ccccc3-c3c4ccccc4c(-c4ccc5oc6cccc7ccc4c5c76)c4ccccc34)cc21 ; CC1(C)c2ccccc2-c2c(-c3ccccc3-c3c4ccccc4c(-c4ccc5oc6cccc7ccc4c5c76)c4ccccc34)cccc21 ; CC1(C)c2ccccc2-c2c(-c3cccc(-c4c5ccccc5c(-c5ccc6oc7cccc8ccc5c6c87)c5ccccc45)c3)cccc21 ; c1ccc2c(c1)cc(-c1ccc(-c3c4ccccc4c(-c4ccc5oc6cccc7ccc4c5c76)c4ccccc34)cc1)c1ccccc12]

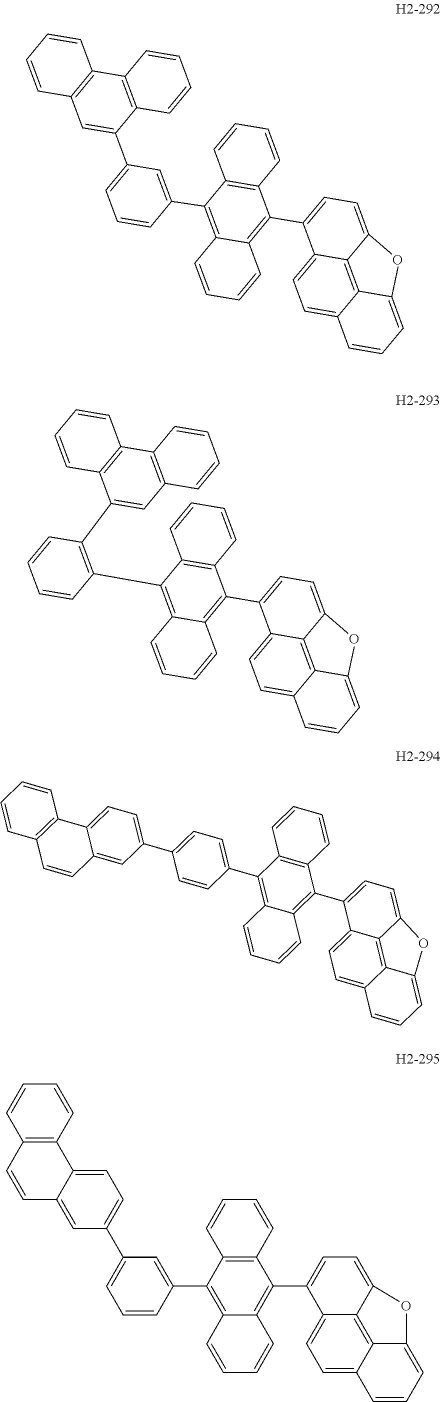 [SMILES: c1cc(-c2c3ccccc3c(-c3ccc4oc5cccc6ccc3c4c65)c3ccccc23)cc(-c2cc3ccccc3c3ccccc23)c1 ; c1ccc(-c2cc3ccccc3c3ccccc23)c(-c2c3ccccc3c(-c3ccc4oc5cccc6ccc3c4c65)c3ccccc23)c1 ; c1ccc2c(c1)ccc1cc(-c3ccc(-c4c5ccccc5c(-c5ccc6oc7cccc8ccc5c6c87)c5ccccc45)cc3)ccc12 ; c1cc(-c2ccc3c(ccc4ccccc43)c2)cc(-c2c3ccccc3c(-c3ccc4oc5cccc6ccc3c4c65)c3ccccc23)c1]

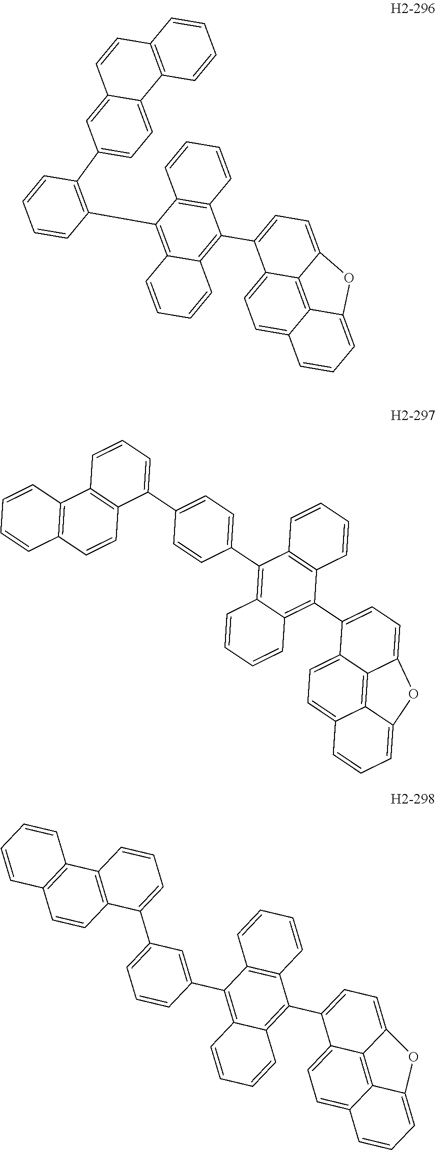 [SMILES: c1ccc(-c2c3ccccc3c(-c3ccc4oc5cccc6ccc3c4c65)c3ccccc23)c(-c2ccc3c(ccc4ccccc43)c2)c1 ; c1ccc2c(c1)ccc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2ccc2ccccc23)c1]

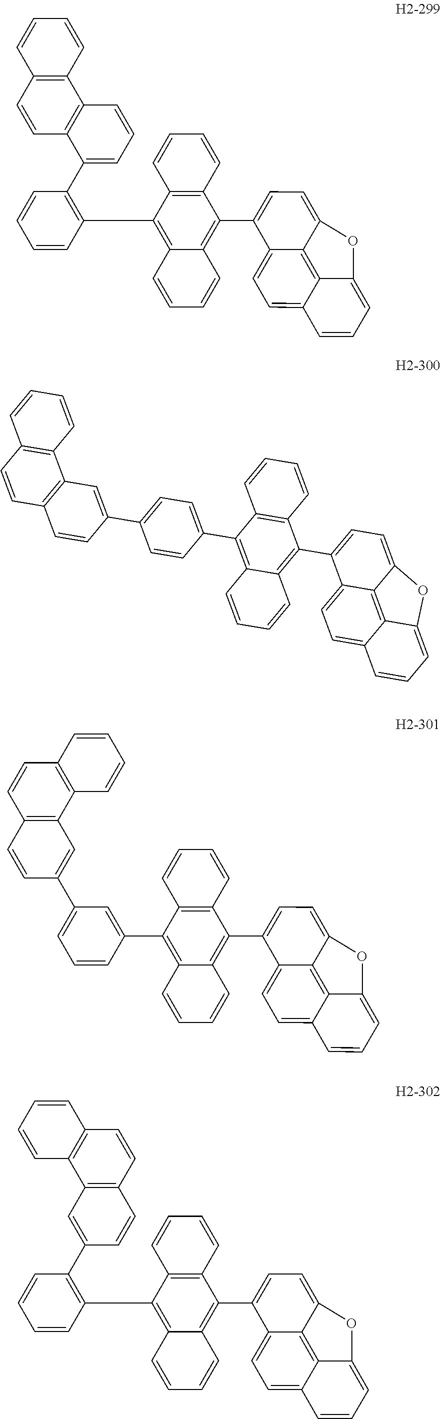 [SMILES: c1ccc(-c2cccc3c2ccc2ccccc23)c(-c2c3ccccc3c(-c3ccc4oc5cccc6ccc3c4c65)c3ccccc23)c1 ; c1ccc2c(c1)ccc1ccc(-c3ccc(-c4c5ccccc5c(-c5ccc6oc7cccc8ccc5c6c87)c5ccccc45)cc3)cc12 ; c1cc(-c2ccc3ccc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccc4ccccc4c3c2)c1]

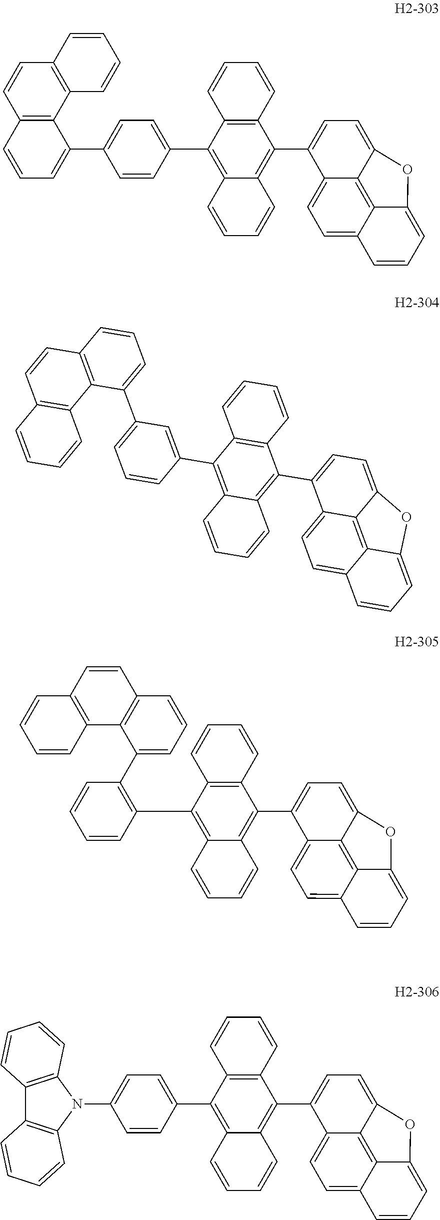 [SMILES: c1ccc2c(c1)ccc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3ccc4ccccc4c23)c1 ; c1ccc(-c2cccc3ccc4ccccc4c23)c(-c2c3ccccc3c(-c3ccc4oc5cccc6ccc3c4c65)c3ccccc23)c1 ; c1cc2ccc3c(-c4c5ccccc5c(-c5ccc(-n6c7ccccc7c7ccccc76)cc5)c5ccccc45)ccc4oc(c1)c2c43]

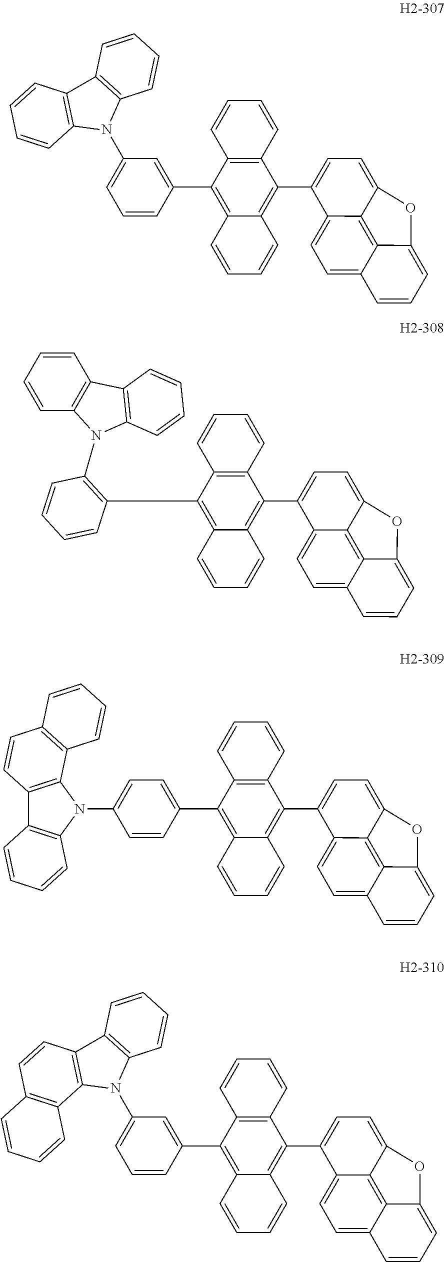 [SMILES: c1cc(-c2c3ccccc3c(-c3ccc4oc5cccc6ccc3c4c65)c3ccccc23)cc(-n2c3ccccc3c3ccccc32)c1 ; c1ccc(-n2c3ccccc3c3ccccc32)c(-c2c3ccccc3c(-c3ccc4oc5cccc6ccc3c4c65)c3ccccc23)c1 ; c1ccc2c(c1)ccc1c3ccccc3n(-c3ccc(-c4c5ccccc5c(-c5ccc6oc7cccc8ccc5c6c87)c5ccccc45)cc3)c21 ; c1cc(-c2c3ccccc3c(-c3ccc4oc5cccc6ccc3c4c65)c3ccccc23)cc(-n2c3ccccc3c3ccc4ccccc4c32)c1]

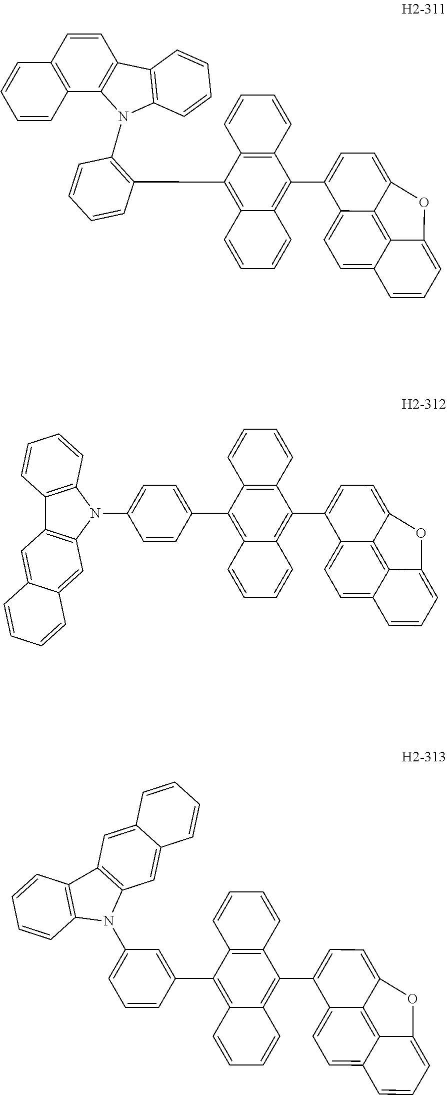 [SMILES: c1ccc(-n2c3ccccc3c3ccc4ccccc4c32)c(-c2c3ccccc3c(-c3ccc4oc5cccc6ccc3c4c65)c3ccccc23)c1 ; c1ccc2cc3c(cc2c1)c1ccccc1n3-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3cc4ccccc4cc32)c1]

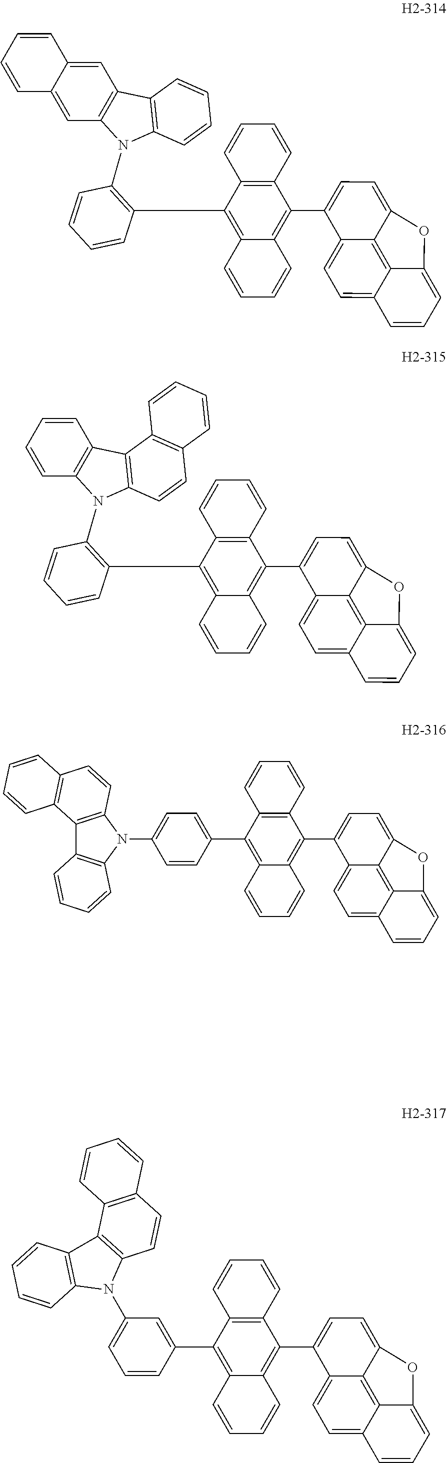 [SMILES: c1ccc(-n2c3ccccc3c3cc4ccccc4cc32)c(-c2c3ccccc3c(-c3ccc4oc5cccc6ccc3c4c65)c3ccccc23)c1 ; c1ccc(-n2c3ccccc3c3c4ccccc4ccc32)c(-c2c3ccccc3c(-c3ccc4oc5cccc6ccc3c4c65)c3ccccc23)c1 ; c1ccc2c(c1)ccc1c2c2ccccc2n1-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3c4ccccc4ccc32)c1]

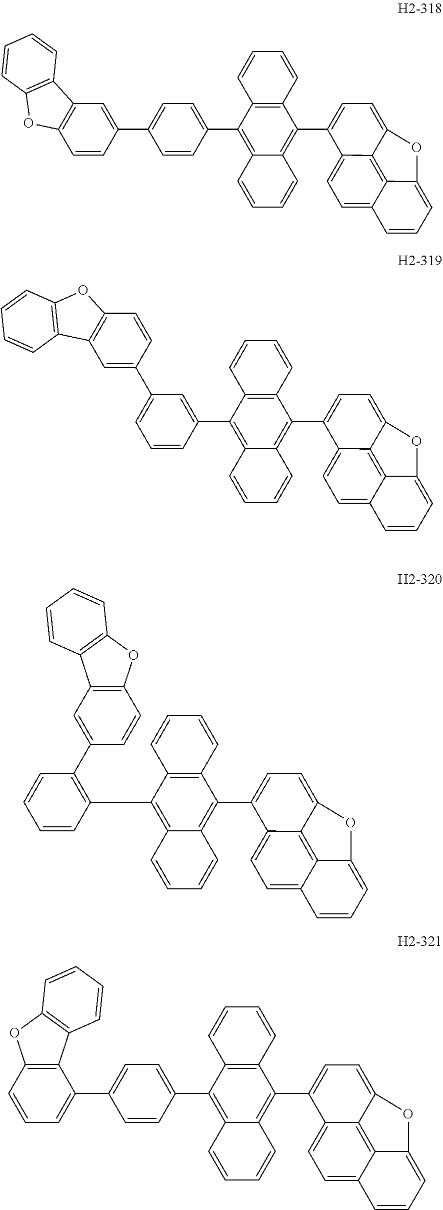 [SMILES: c1ccc2c(c1)oc1ccc(-c3ccc(-c4c5ccccc5c(-c5ccc6oc7cccc8ccc5c6c87)c5ccccc45)cc3)cc12 ; c1cc(-c2ccc3oc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3oc4ccccc4c3c2)c1 ; c1ccc2c(c1)oc1cccc(-c3ccc(-c4c5ccccc5c(-c5ccc6oc7cccc8ccc5c6c87)c5ccccc45)cc3)c12]

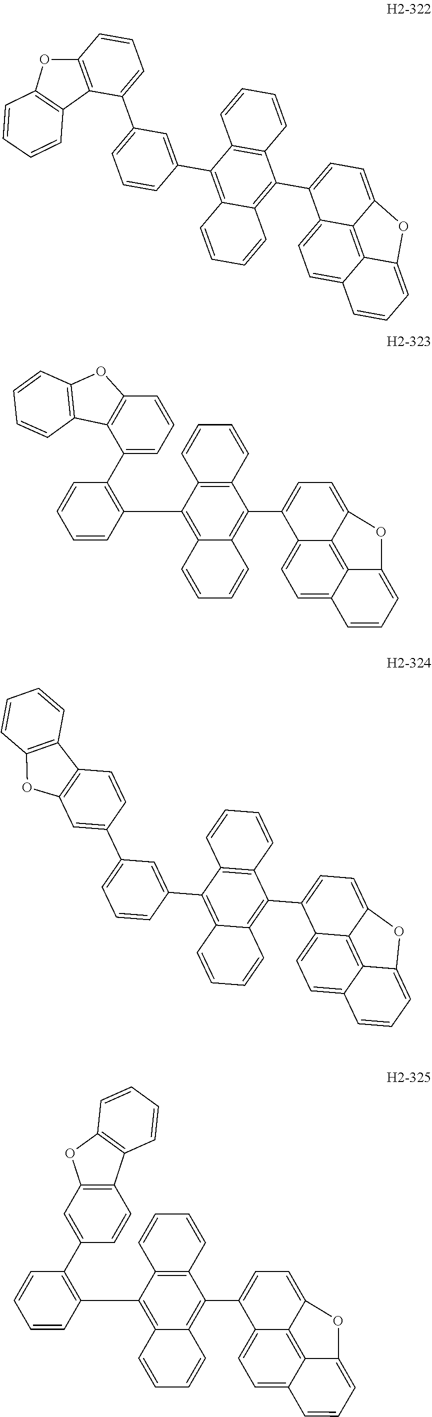 [SMILES: c1cc(-c2c3ccccc3c(-c3ccc4oc5cccc6ccc3c4c65)c3ccccc23)cc(-c2cccc3oc4ccccc4c23)c1 ; c1ccc(-c2cccc3oc4ccccc4c23)c(-c2c3ccccc3c(-c3ccc4oc5cccc6ccc3c4c65)c3ccccc23)c1 ; c1cc(-c2ccc3c(c2)oc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)oc2ccccc23)c1]

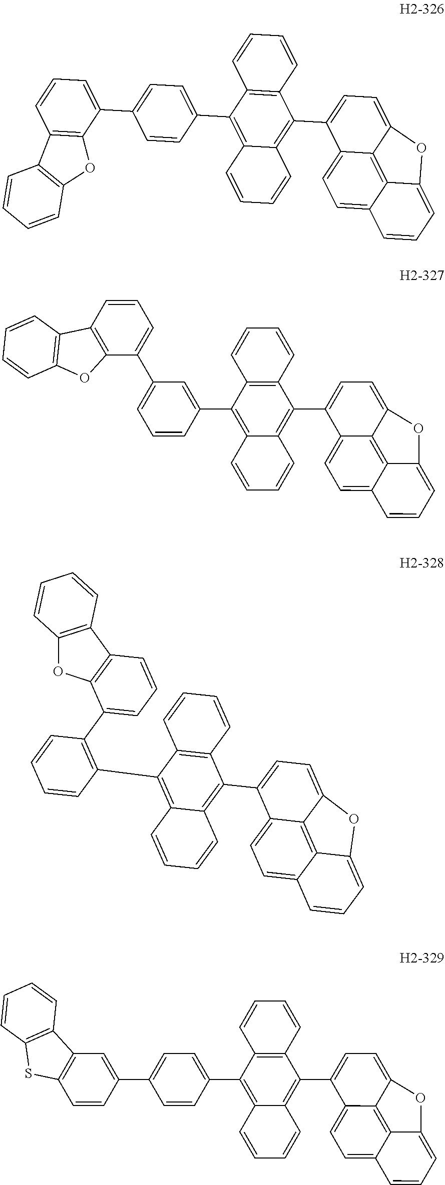 [SMILES: c1ccc2c(c1)oc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2oc2ccccc23)c1 ; c1ccc(-c2cccc3c2oc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6ccc7sc8ccccc8c7c6)cc5)c5ccccc45)ccc4oc(c1)c2c43]

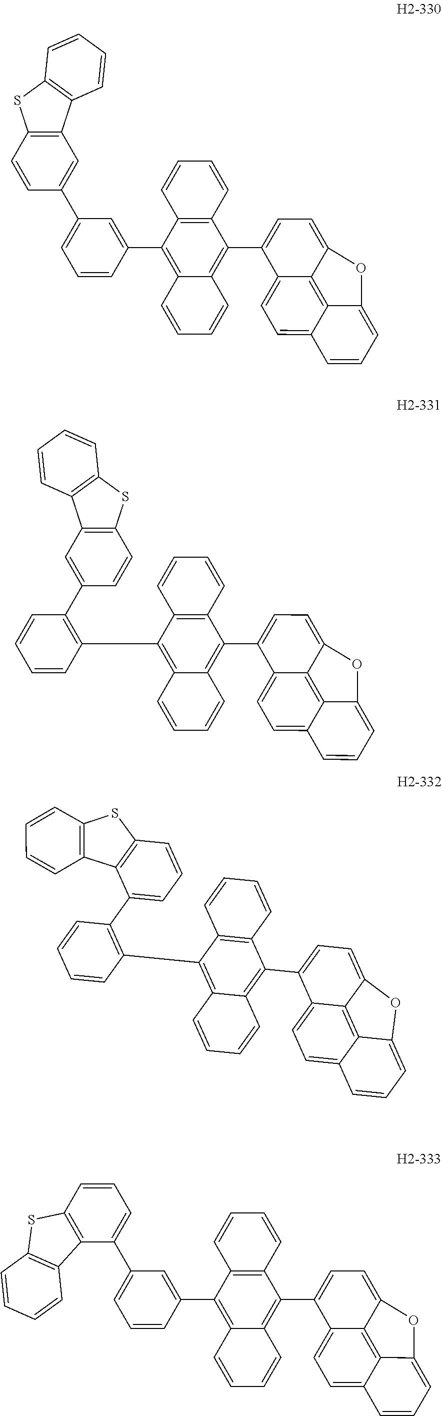 [SMILES: c1cc(-c2ccc3sc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3sc4ccccc4c3c2)c1 ; c1ccc(-c2cccc3sc4ccccc4c23)c(-c2c3ccccc3c(-c3ccc4oc5cccc6ccc3c4c65)c3ccccc23)c1 ; c1cc(-c2c3ccccc3c(-c3ccc4oc5cccc6ccc3c4c65)c3ccccc23)cc(-c2cccc3sc4ccccc4c23)c1]

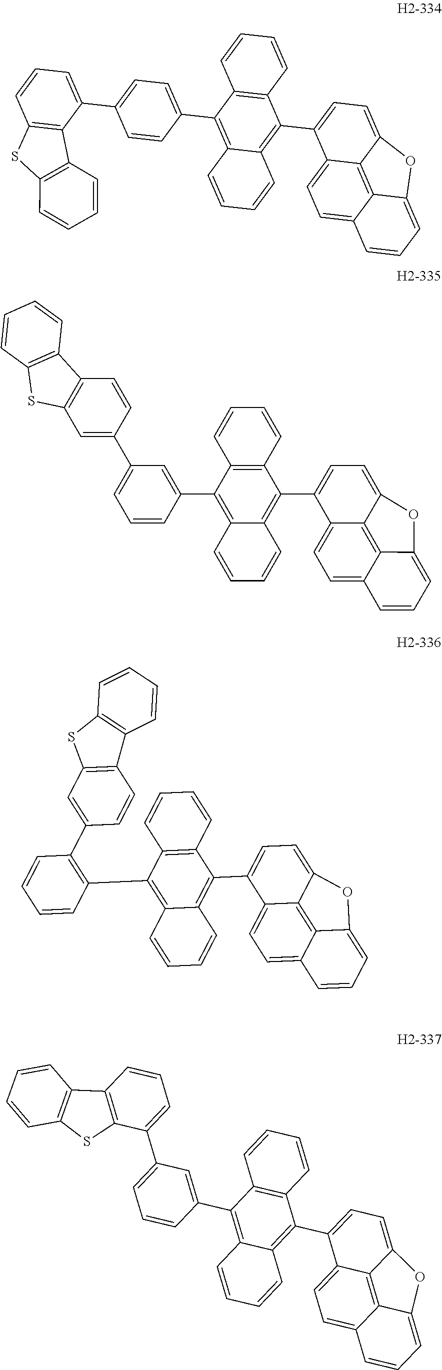 [SMILES: c1cc2ccc3c(-c4c5ccccc5c(-c5ccc(-c6cccc7sc8ccccc8c67)cc5)c5ccccc45)ccc4oc(c1)c2c43 ; c1cc(-c2ccc3c(c2)sc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)sc2ccccc23)c1 ; c1cc(-c2c3ccccc3c(-c3ccc4oc5cccc6ccc3c4c65)c3ccccc23)cc(-c2cccc3c2sc2ccccc23)c1]

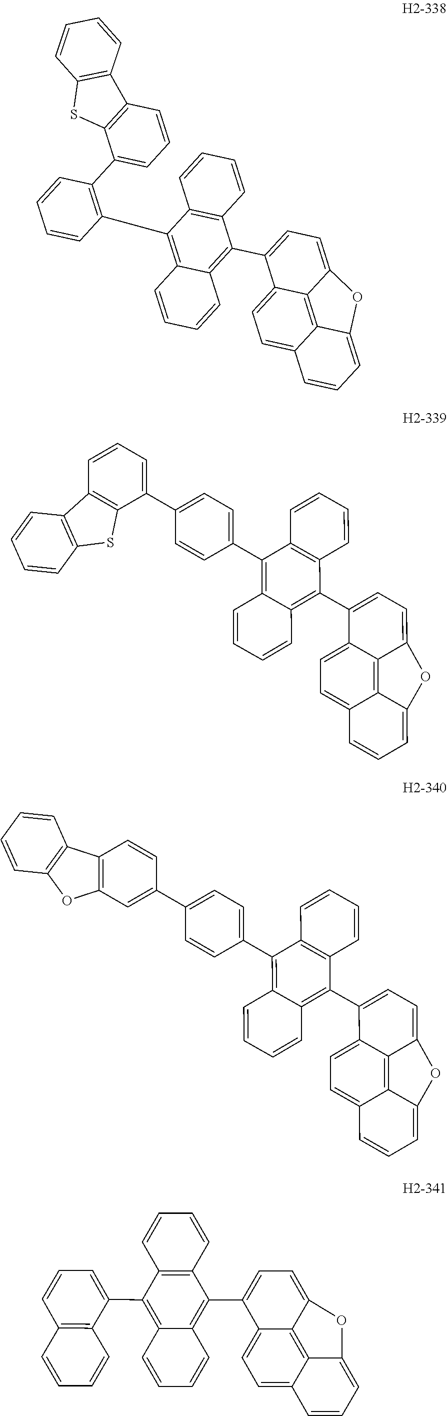 [SMILES: c1ccc(-c2cccc3c2sc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6cccc7c6sc6ccccc67)cc5)c5ccccc45)ccc4oc(c1)c2c43 ; c1ccc2c(c1)oc1cc(-c3ccc(-c4c5ccccc5c(-c5ccc6oc7cccc8ccc5c6c87)c5ccccc45)cc3)ccc12 ; c1ccc2c(-c3c4ccccc4c(-c4ccc5oc6cccc7ccc4c5c76)c4ccccc34)cccc2c1]

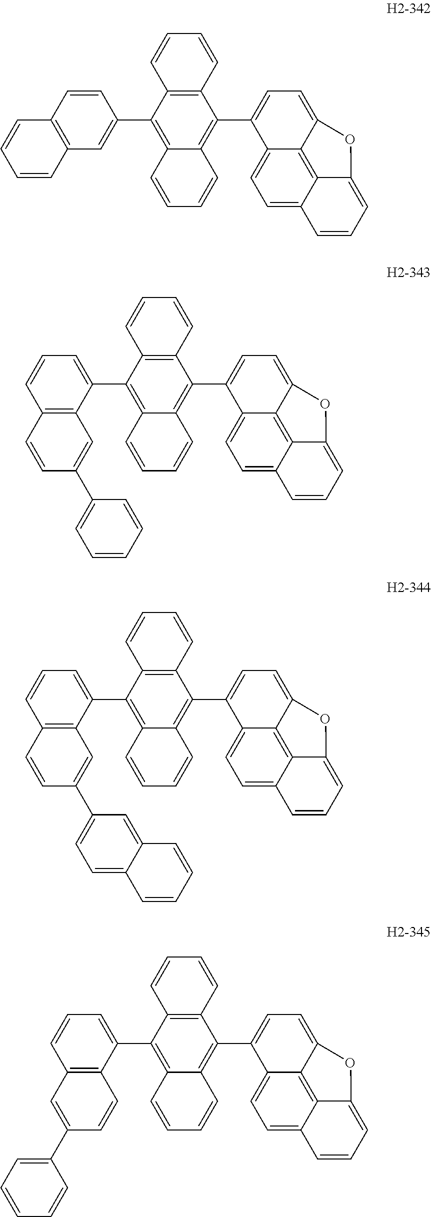 [SMILES: c1ccc2cc(-c3c4ccccc4c(-c4ccc5oc6cccc7ccc4c5c76)c4ccccc34)ccc2c1 ; c1ccc(-c2ccc3cccc(-c4c5ccccc5c(-c5ccc6oc7cccc8ccc5c6c87)c5ccccc45)c3c2)cc1 ; c1ccc2cc(-c3ccc4cccc(-c5c6ccccc6c(-c6ccc7oc8cccc9ccc6c7c98)c6ccccc56)c4c3)ccc2c1 ; c1ccc(-c2ccc3c(-c4c5ccccc5c(-c5ccc6oc7cccc8ccc5c6c87)c5ccccc45)cccc3c2)cc1]

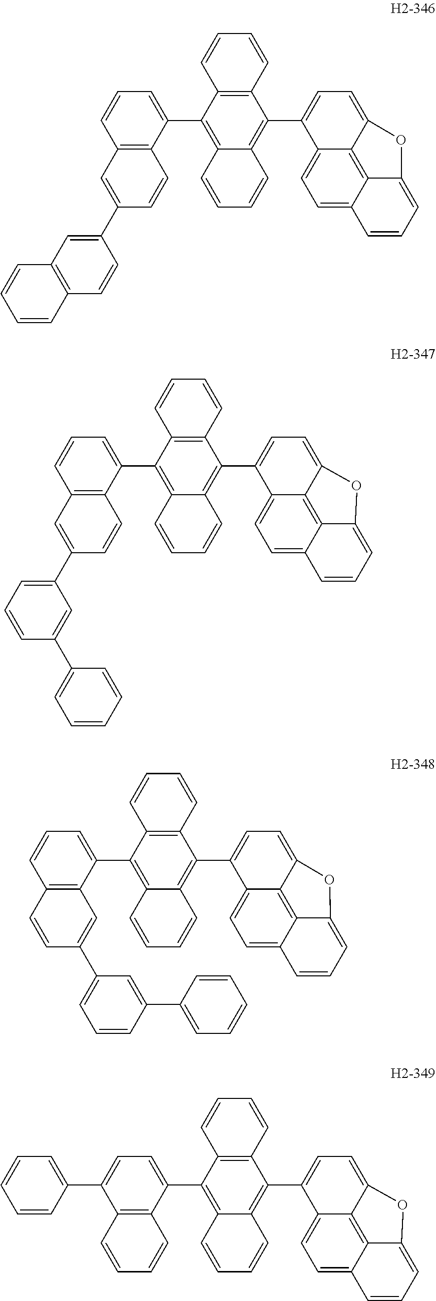 [SMILES: c1ccc2cc(-c3ccc4c(-c5c6ccccc6c(-c6ccc7oc8cccc9ccc6c7c98)c6ccccc56)cccc4c3)ccc2c1 ; c1ccc(-c2cccc(-c3ccc4c(-c5c6ccccc6c(-c6ccc7oc8cccc9ccc6c7c98)c6ccccc56)cccc4c3)c2)cc1 ; c1ccc(-c2cccc(-c3ccc4cccc(-c5c6ccccc6c(-c6ccc7oc8cccc9ccc6c7c98)c6ccccc56)c4c3)c2)cc1 ; c1ccc(-c2ccc(-c3c4ccccc4c(-c4ccc5oc6cccc7ccc4c5c76)c4ccccc34)c3ccccc23)cc1]

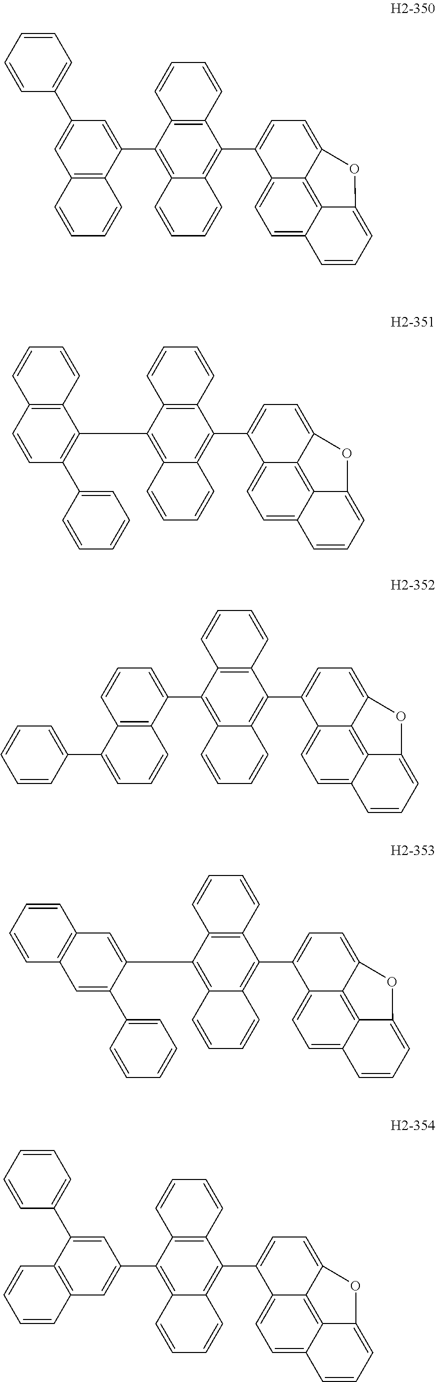 [SMILES: c1ccc(-c2cc(-c3c4ccccc4c(-c4ccc5oc6cccc7ccc4c5c76)c4ccccc34)c3ccccc3c2)cc1 ; c1ccc(-c2ccc3ccccc3c2-c2c3ccccc3c(-c3ccc4oc5cccc6ccc3c4c65)c3ccccc23)cc1 ; c1ccc(-c2cccc3c(-c4c5ccccc5c(-c5ccc6oc7cccc8ccc5c6c87)c5ccccc45)cccc23)cc1 ; c1ccc(-c2cc3ccccc3cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)cc3ccccc23)cc1]

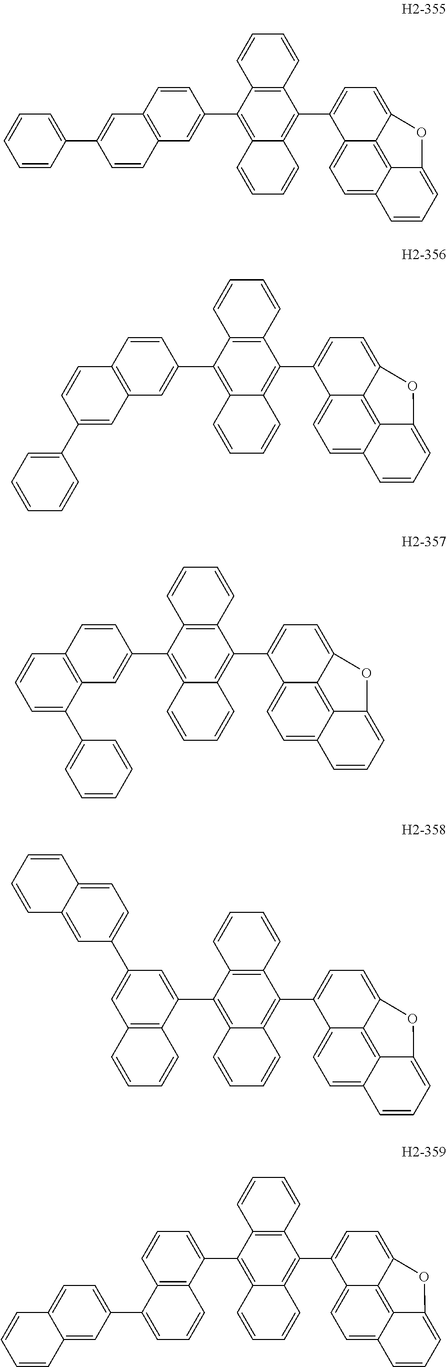 [SMILES: c1ccc(-c2ccc3cc(-c4c5ccccc5c(-c5ccc6oc7cccc8ccc5c6c87)c5ccccc45)ccc3c2)cc1 ; c1ccc(-c2ccc3ccc(-c4c5ccccc5c(-c5ccc6oc7cccc8ccc5c6c87)c5ccccc45)cc3c2)cc1 ; c1ccc(-c2cccc3ccc(-c4c5ccccc5c(-c5ccc6oc7cccc8ccc5c6c87)c5ccccc45)cc23)cc1 ; c1ccc2cc(-c3cc(-c4c5ccccc5c(-c5ccc6oc7cccc8ccc5c6c87)c5ccccc45)c4ccccc4c3)ccc2c1 ; c1ccc2cc(-c3cccc4c(-c5c6ccccc6c(-c6ccc7oc8cccc9ccc6c7c98)c6ccccc56)cccc34)ccc2c1]

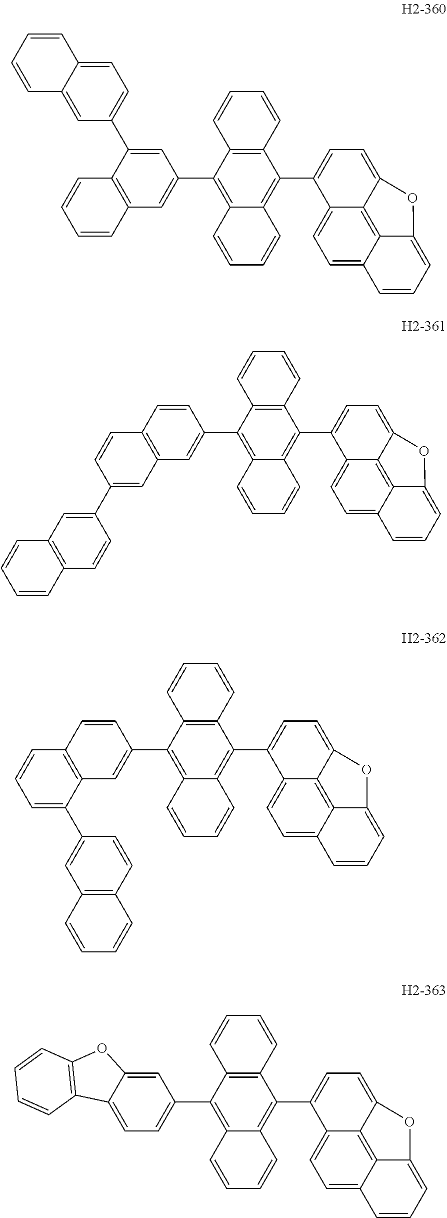 [SMILES: c1ccc2cc(-c3cc(-c4c5ccccc5c(-c5ccc6oc7cccc8ccc5c6c87)c5ccccc45)cc4ccccc34)ccc2c1 ; c1ccc2cc(-c3ccc4ccc(-c5c6ccccc6c(-c6ccc7oc8cccc9ccc6c7c98)c6ccccc56)cc4c3)ccc2c1 ; c1ccc2cc(-c3cccc4ccc(-c5c6ccccc6c(-c6ccc7oc8cccc9ccc6c7c98)c6ccccc56)cc34)ccc2c1 ; c1ccc2c(c1)oc1cc(-c3c4ccccc4c(-c4ccc5oc6cccc7ccc4c5c76)c4ccccc34)ccc12]

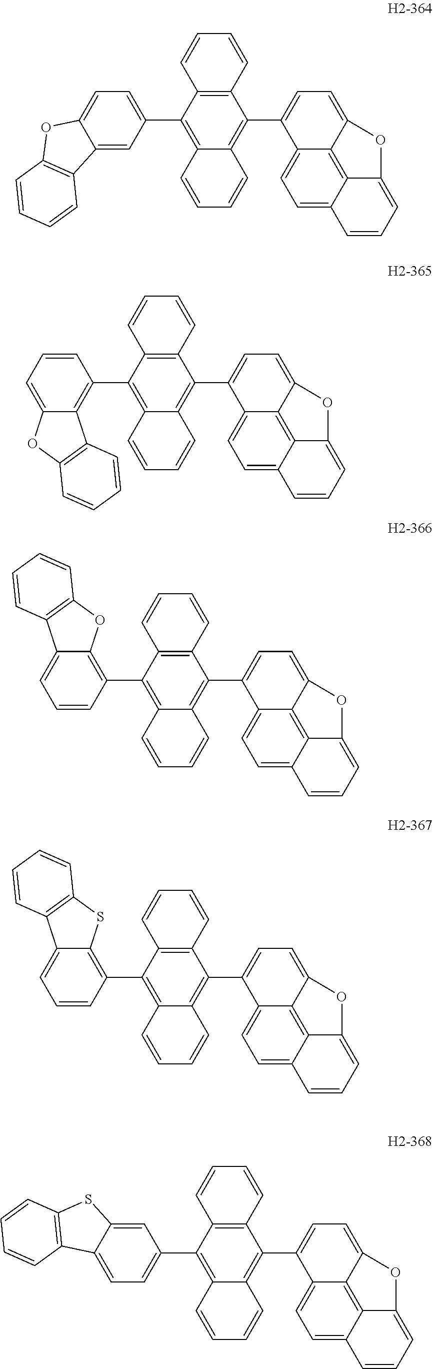 [SMILES: c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12 ; c1ccc2c(c1)oc1c(-c3c4ccccc4c(-c4ccc5oc6cccc7ccc4c5c76)c4ccccc34)cccc12 ; c1cc2ccc3c(-c4c5ccccc5c(-c5cccc6c5sc5ccccc56)c5ccccc45)ccc4oc(c1)c2c43 ; c1cc2ccc3c(-c4c5ccccc5c(-c5ccc6c(c5)sc5ccccc56)c5ccccc45)ccc4oc(c1)c2c43]

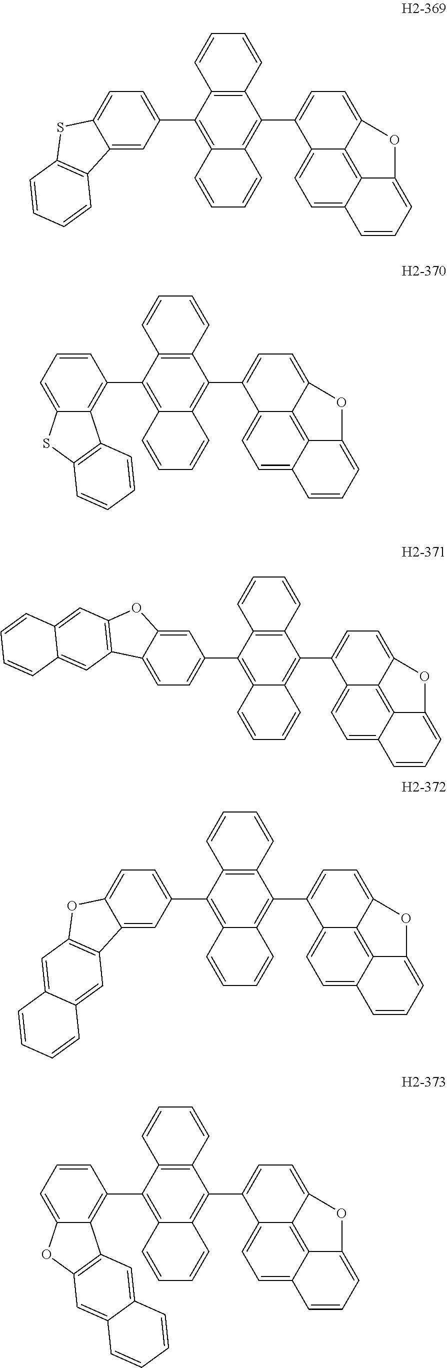 [SMILES: c1cc2ccc3c(-c4c5ccccc5c(-c5ccc6sc7ccccc7c6c5)c5ccccc45)ccc4oc(c1)c2c43 ; c1cc2ccc3c(-c4c5ccccc5c(-c5cccc6sc7ccccc7c56)c5ccccc45)ccc4oc(c1)c2c43 ; c1ccc2cc3c(cc2c1)oc1cc(-c2c4ccccc4c(-c4ccc5oc6cccc7ccc4c5c76)c4ccccc24)ccc13 ; c1ccc2cc3c(cc2c1)oc1ccc(-c2c4ccccc4c(-c4ccc5oc6cccc7ccc4c5c76)c4ccccc24)cc13 ; c1ccc2cc3c(cc2c1)oc1cccc(-c2c4ccccc4c(-c4ccc5oc6cccc7ccc4c5c76)c4ccccc24)c13]

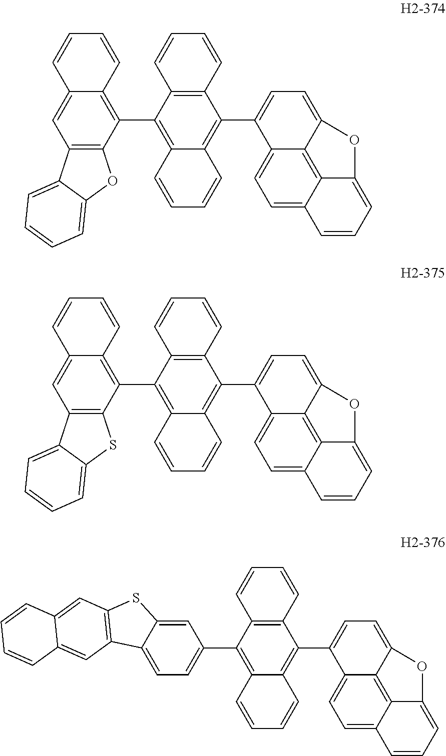 [SMILES: c1ccc2c(-c3c4ccccc4c(-c4ccc5oc6cccc7ccc4c5c76)c4ccccc34)c3oc4ccccc4c3cc2c1 ; c1ccc2c(-c3c4ccccc4c(-c4ccc5oc6cccc7ccc4c5c76)c4ccccc34)c3sc4ccccc4c3cc2c1 ; c1ccc2cc3c(cc2c1)sc1cc(-c2c4ccccc4c(-c4ccc5oc6cccc7ccc4c5c76)c4ccccc24)ccc13]

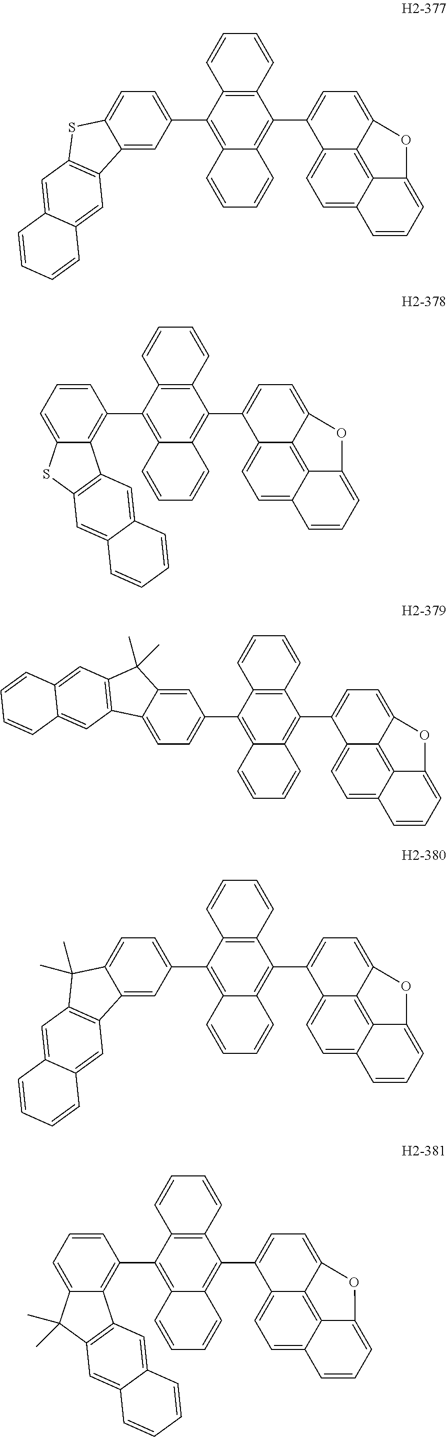 [SMILES: c1ccc2cc3c(cc2c1)sc1ccc(-c2c4ccccc4c(-c4ccc5oc6cccc7ccc4c5c76)c4ccccc24)cc13 ; c1ccc2cc3c(cc2c1)sc1cccc(-c2c4ccccc4c(-c4ccc5oc6cccc7ccc4c5c76)c4ccccc24)c13 ; CC1(C)c2cc(-c3c4ccccc4c(-c4ccc5oc6cccc7ccc4c5c76)c4ccccc34)ccc2-c2cc3ccccc3cc21 ; CC1(C)c2ccc(-c3c4ccccc4c(-c4ccc5oc6cccc7ccc4c5c76)c4ccccc34)cc2-c2cc3ccccc3cc21 ; CC1(C)c2cc3ccccc3cc2-c2c(-c3c4ccccc4c(-c4ccc5oc6cccc7ccc4c5c76)c4ccccc34)cccc21]

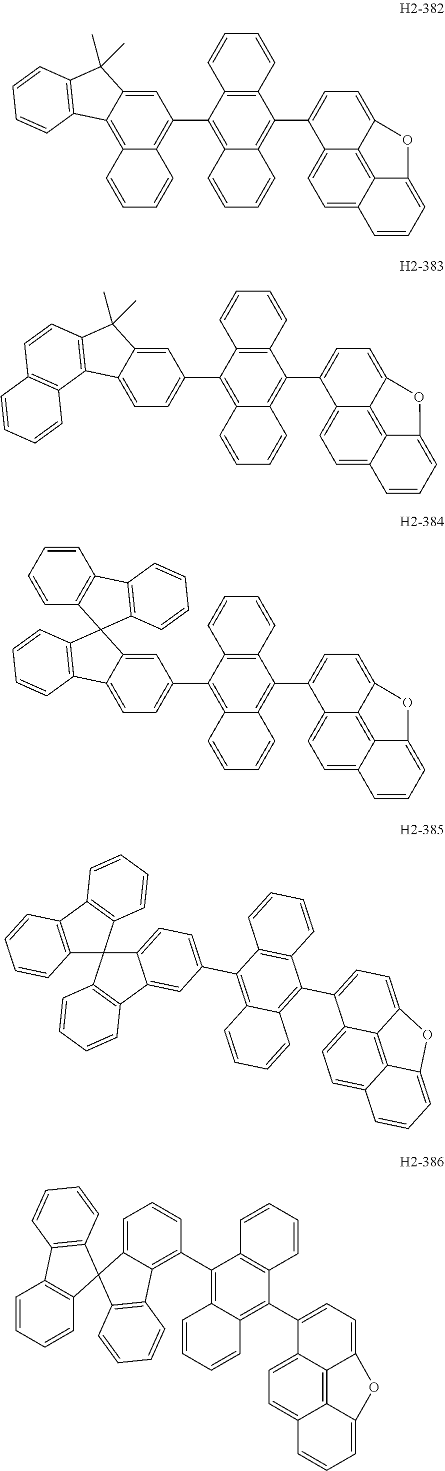 [SMILES: CC1(C)c2ccccc2-c2c1cc(-c1c3ccccc3c(-c3ccc4oc5cccc6ccc3c4c65)c3ccccc13)c1ccccc21 ; CC1(C)c2cc(-c3c4ccccc4c(-c4ccc5oc6cccc7ccc4c5c76)c4ccccc34)ccc2-c2c1ccc1ccccc21 ; c1ccc2c(c1)-c1ccccc1C21c2ccccc2-c2ccc(-c3c4ccccc4c(-c4ccc5oc6cccc7ccc4c5c76)c4ccccc34)cc21 ; c1ccc2c(c1)-c1ccccc1C21c2ccccc2-c2cc(-c3c4ccccc4c(-c4ccc5oc6cccc7ccc4c5c76)c4ccccc34)ccc21 ; c1ccc2c(c1)-c1ccccc1C21c2ccccc2-c2c(-c3c4ccccc4c(-c4ccc5oc6cccc7ccc4c5c76)c4ccccc34)cccc21]

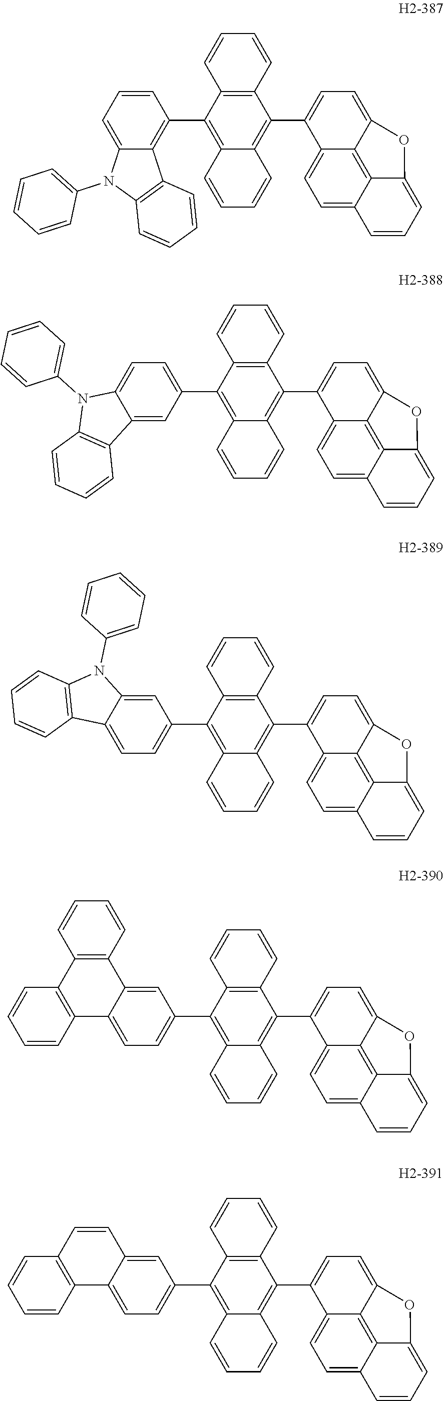 [SMILES: c1ccc(-n2c3ccccc3c3c(-c4c5ccccc5c(-c5ccc6oc7cccc8ccc5c6c87)c5ccccc45)cccc32)cc1 ; c1ccc(-n2c3ccccc3c3cc(-c4c5ccccc5c(-c5ccc6oc7cccc8ccc5c6c87)c5ccccc45)ccc32)cc1 ; c1ccc(-n2c3ccccc3c3ccc(-c4c5ccccc5c(-c5ccc6oc7cccc8ccc5c6c87)c5ccccc45)cc32)cc1 ; c1cc2ccc3c(-c4c5ccccc5c(-c5ccc6c7ccccc7c7ccccc7c6c5)c5ccccc45)ccc4oc(c1)c2c43 ; c1ccc2c(c1)ccc1cc(-c3c4ccccc4c(-c4ccc5oc6cccc7ccc4c5c76)c4ccccc34)ccc12]

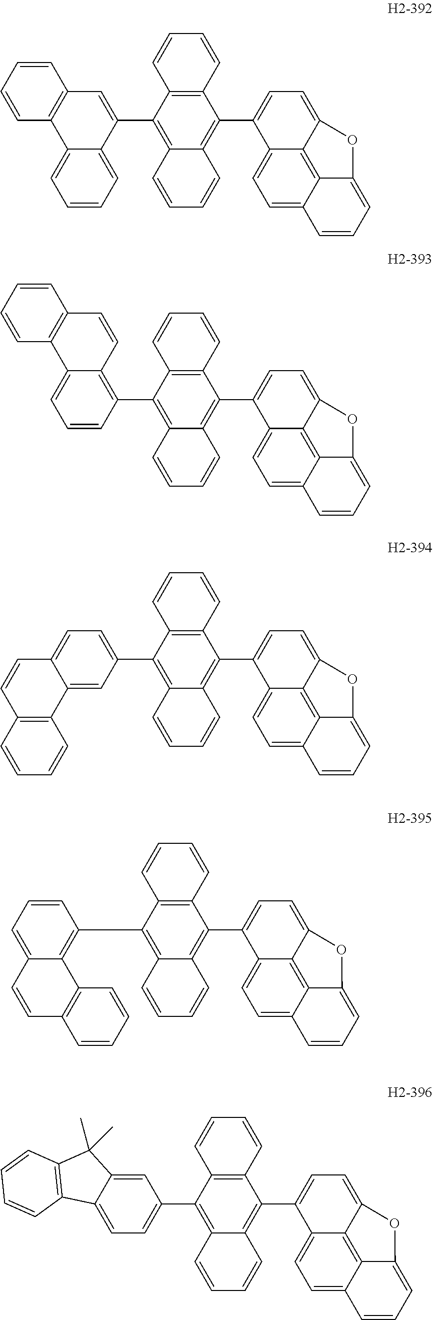 [SMILES: c1ccc2c(c1)cc(-c1c3ccccc3c(-c3ccc4oc5cccc6ccc3c4c65)c3ccccc13)c1ccccc12 ; c1ccc2c(c1)ccc1c(-c3c4ccccc4c(-c4ccc5oc6cccc7ccc4c5c76)c4ccccc34)cccc12 ; c1ccc2c(c1)ccc1ccc(-c3c4ccccc4c(-c4ccc5oc6cccc7ccc4c5c76)c4ccccc34)cc12 ; c1ccc2c(c1)ccc1cccc(-c3c4ccccc4c(-c4ccc5oc6cccc7ccc4c5c76)c4ccccc34)c12 ; CC1(C)c2ccccc2-c2ccc(-c3c4ccccc4c(-c4ccc5oc6cccc7ccc4c5c76)c4ccccc34)cc21]

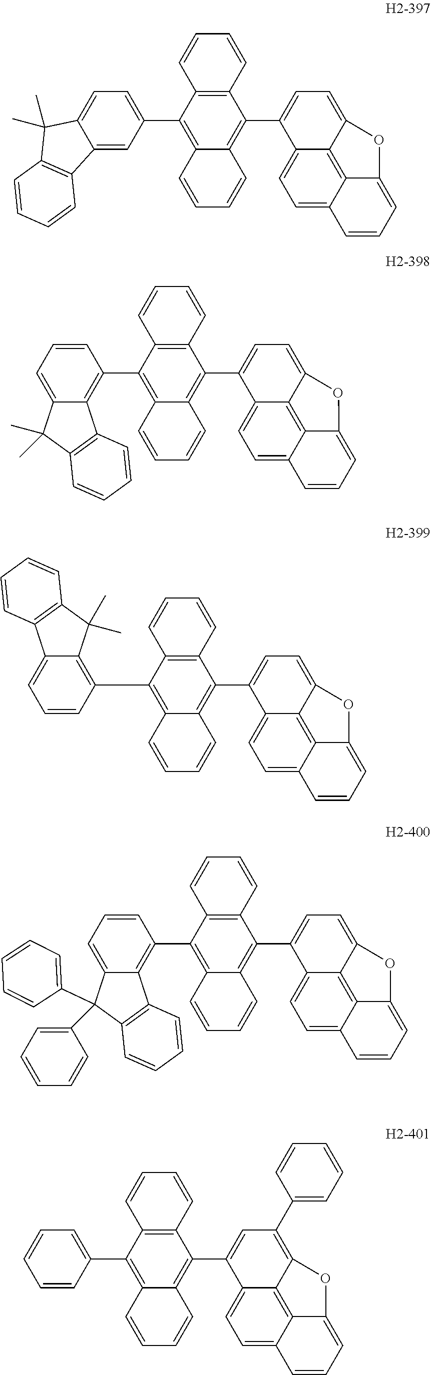 [SMILES: CC1(C)c2ccccc2-c2cc(-c3c4ccccc4c(-c4ccc5oc6cccc7ccc4c5c76)c4ccccc34)ccc21 ; CC1(C)c2ccccc2-c2c(-c3c4ccccc4c(-c4ccc5oc6cccc7ccc4c5c76)c4ccccc34)cccc21 ; CC1(C)c2ccccc2-c2cccc(-c3c4ccccc4c(-c4ccc5oc6cccc7ccc4c5c76)c4ccccc34)c21 ; c1ccc(C2(c3ccccc3)c3ccccc3-c3c(-c4c5ccccc5c(-c5ccc6oc7cccc8ccc5c6c87)c5ccccc45)cccc32)cc1 ; c1ccc(-c2c3ccccc3c(-c3cc(-c4ccccc4)c4oc5cccc6ccc3c4c65)c3ccccc23)cc1]

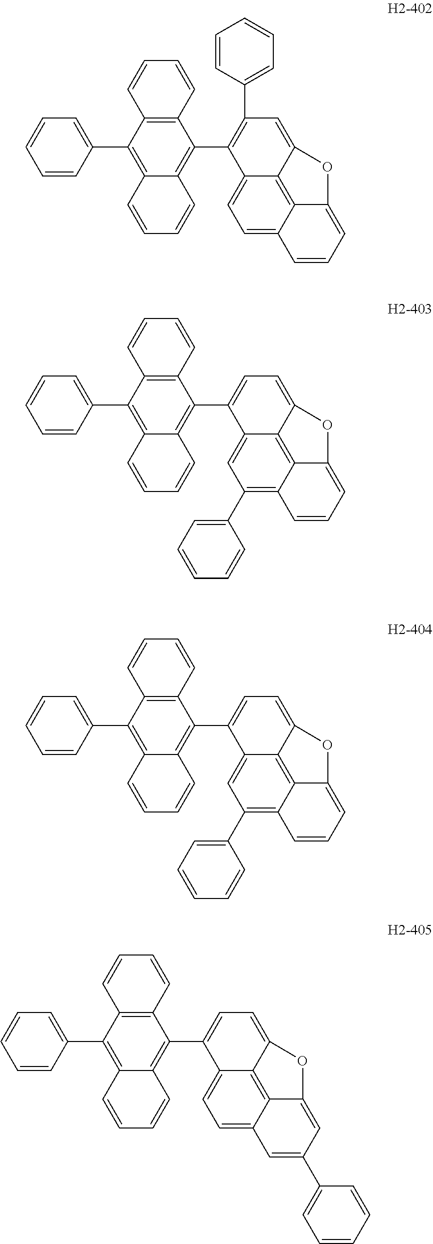 [SMILES: c1ccc(-c2cc3oc4cccc5ccc(c2-c2c6ccccc6c(-c6ccccc6)c6ccccc26)c3c54)cc1 ; c1ccc(-c2c3ccccc3c(-c3ccc4oc5cccc6c(-c7ccccc7)cc3c4c56)c3ccccc23)cc1 ; c1ccc(-c2c3ccccc3c(-c3ccc4oc5cccc6c(-c7ccccc7)cc3c4c56)c3ccccc23)cc1 ; c1ccc(-c2cc3ccc4c(-c5c6ccccc6c(-c6ccccc6)c6ccccc56)ccc5oc(c2)c3c54)cc1]

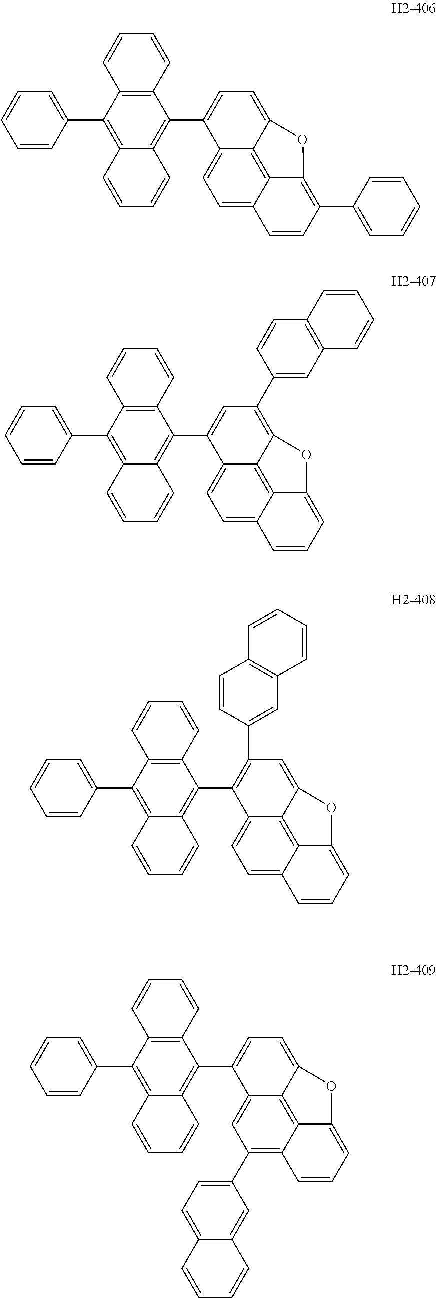 [SMILES: c1ccc(-c2c3ccccc3c(-c3ccc4oc5c(-c6ccccc6)ccc6ccc3c4c65)c3ccccc23)cc1 ; c1ccc(-c2c3ccccc3c(-c3cc(-c4ccc5ccccc5c4)c4oc5cccc6ccc3c4c65)c3ccccc23)cc1 ; c1ccc(-c2c3ccccc3c(-c3c(-c4ccc5ccccc5c4)cc4oc5cccc6ccc3c4c65)c3ccccc23)cc1 ; c1ccc(-c2c3ccccc3c(-c3ccc4oc5cccc6c(-c7ccc8ccccc8c7)cc3c4c56)c3ccccc23)cc1]

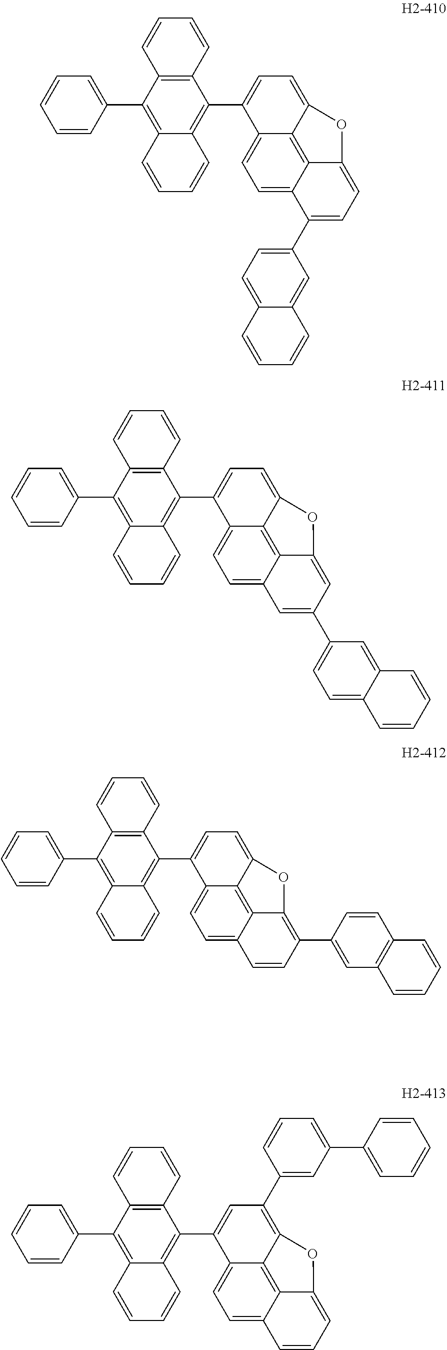 [SMILES: c1ccc(-c2c3ccccc3c(-c3ccc4oc5ccc(-c6ccc7ccccc7c6)c6ccc3c4c56)c3ccccc23)cc1 ; c1ccc(-c2c3ccccc3c(-c3ccc4oc5cc(-c6ccc7ccccc7c6)cc6ccc3c4c65)c3ccccc23)cc1 ; c1ccc(-c2c3ccccc3c(-c3ccc4oc5c(-c6ccc7ccccc7c6)ccc6ccc3c4c65)c3ccccc23)cc1 ; c1ccc(-c2cccc(-c3cc(-c4c5ccccc5c(-c5ccccc5)c5ccccc45)c4ccc5cccc6oc3c4c56)c2)cc1]

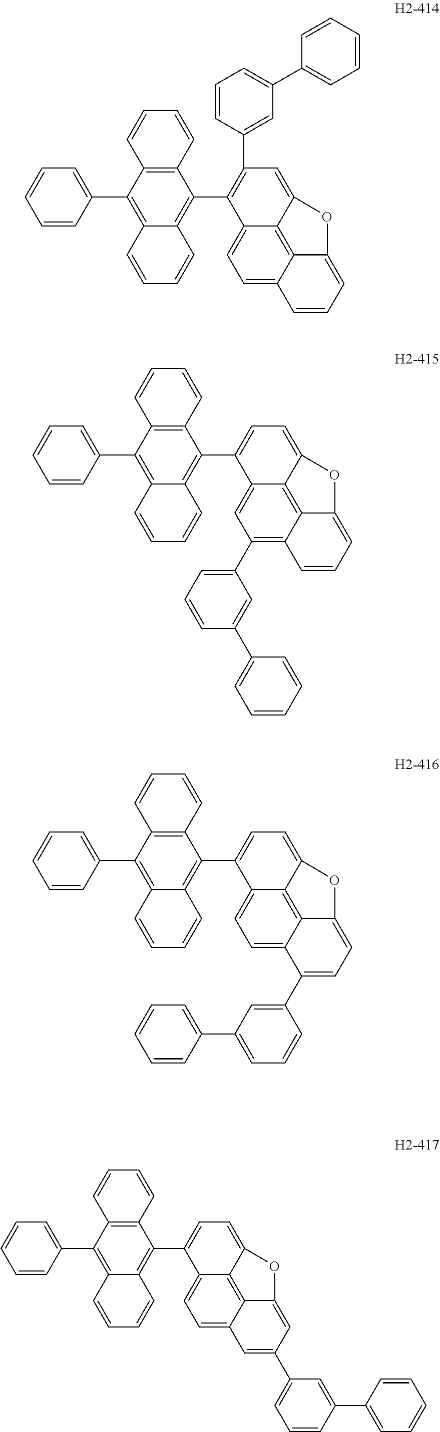 [SMILES: c1ccc(-c2cccc(-c3cc4oc5cccc6ccc(c3-c3c7ccccc7c(-c7ccccc7)c7ccccc37)c4c65)c2)cc1 ; c1ccc(-c2cccc(-c3cc4c(-c5c6ccccc6c(-c6ccccc6)c6ccccc56)ccc5oc6cccc3c6c54)c2)cc1 ; c1ccc(-c2cccc(-c3ccc4oc5ccc(-c6c7ccccc7c(-c7ccccc7)c7ccccc67)c6ccc3c4c56)c2)cc1 ; c1ccc(-c2cccc(-c3cc4ccc5c(-c6c7ccccc7c(-c7ccccc7)c7ccccc67)ccc6oc(c3)c4c65)c2)cc1]

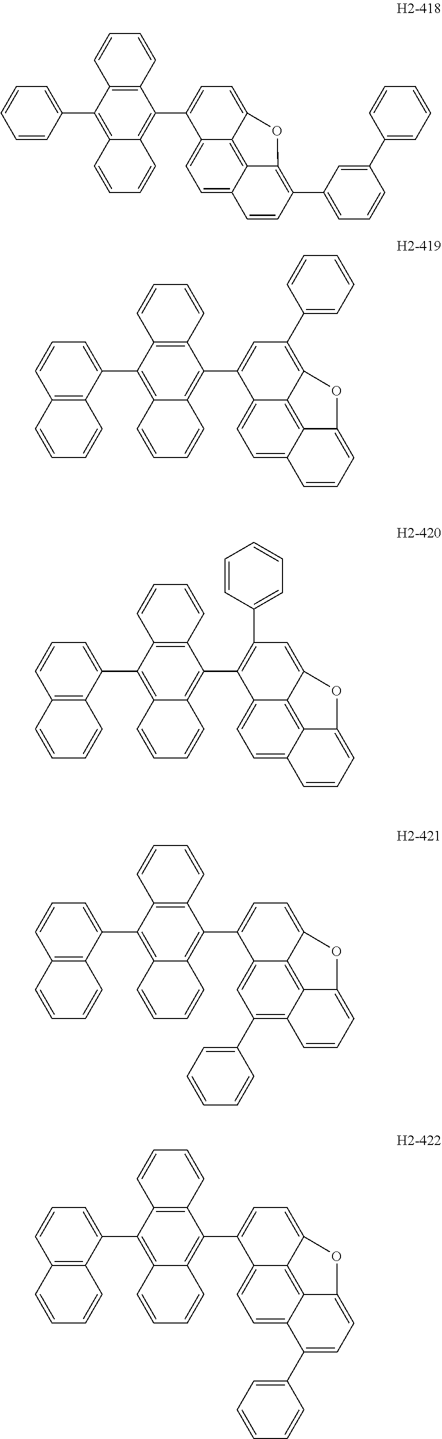

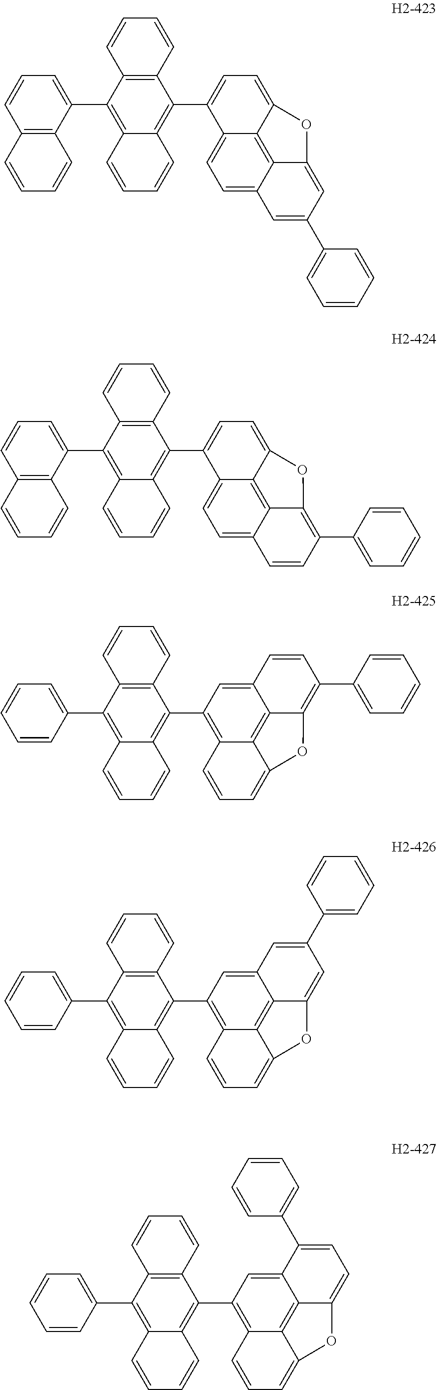 [SMILES: c1ccc(-c2cc3ccc4c(-c5c6ccccc6c(-c6cccc7ccccc67)c6ccccc56)ccc5oc(c2)c3c54)cc1 ; c1ccc(-c2ccc3ccc4c(-c5c6ccccc6c(-c6cccc7ccccc67)c6ccccc56)ccc5oc2c3c54)cc1 ; c1ccc(-c2c3ccccc3c(-c3cc4ccc(-c5ccccc5)c5oc6cccc3c6c45)c3ccccc23)cc1 ; c1ccc(-c2cc3cc(-c4c5ccccc5c(-c5ccccc5)c5ccccc45)c4cccc5oc(c2)c3c54)cc1 ; c1ccc(-c2c3ccccc3c(-c3cc4c(-c5ccccc5)ccc5oc6cccc3c6c54)c3ccccc23)cc1]

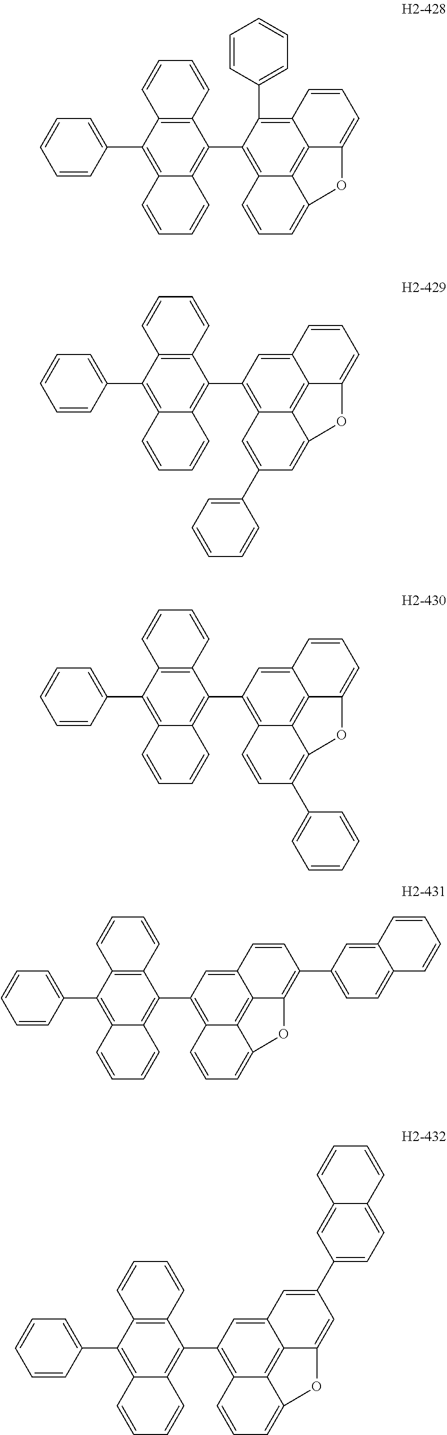 [SMILES: c1ccc(-c2c3ccccc3c(-c3c(-c4ccccc4)c4cccc5oc6cccc3c6c54)c3ccccc23)cc1 ; c1ccc(-c2cc3oc4cccc5cc(-c6c7ccccc7c(-c7ccccc7)c7ccccc67)c(c2)c3c54)cc1 ; c1ccc(-c2c3ccccc3c(-c3cc4cccc5oc6c(-c7ccccc7)ccc3c6c45)c3ccccc23)cc1 ; c1ccc(-c2c3ccccc3c(-c3cc4ccc(-c5ccc6ccccc6c5)c5oc6cccc3c6c45)c3ccccc23)cc1 ; c1ccc(-c2c3ccccc3c(-c3cc4cc(-c5ccc6ccccc6c5)cc5oc6cccc3c6c45)c3ccccc23)cc1]

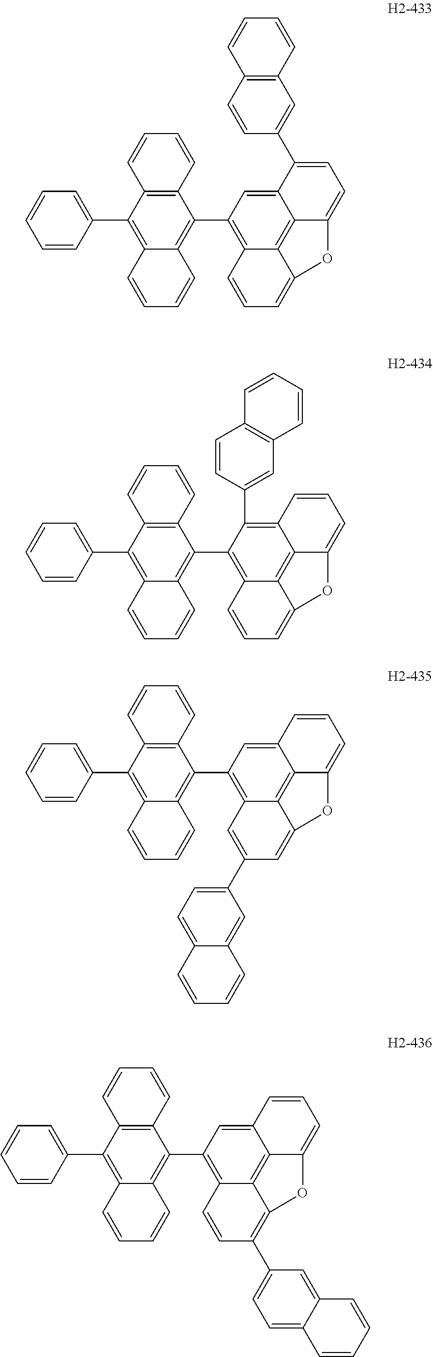 [SMILES: c1ccc(-c2c3ccccc3c(-c3cc4c(-c5ccc6ccccc6c5)ccc5oc6cccc3c6c54)c3ccccc23)cc1 ; c1ccc(-c2c3ccccc3c(-c3c(-c4ccc5ccccc5c4)c4cccc5oc6cccc3c6c54)c3ccccc23)cc1 ; c1ccc(-c2c3ccccc3c(-c3cc4cccc5oc6cc(-c7ccc8ccccc8c7)cc3c6c45)c3ccccc23)cc1 ; c1ccc(-c2c3ccccc3c(-c3cc4cccc5oc6c(-c7ccc8ccccc8c7)ccc3c6c45)c3ccccc23)cc1]

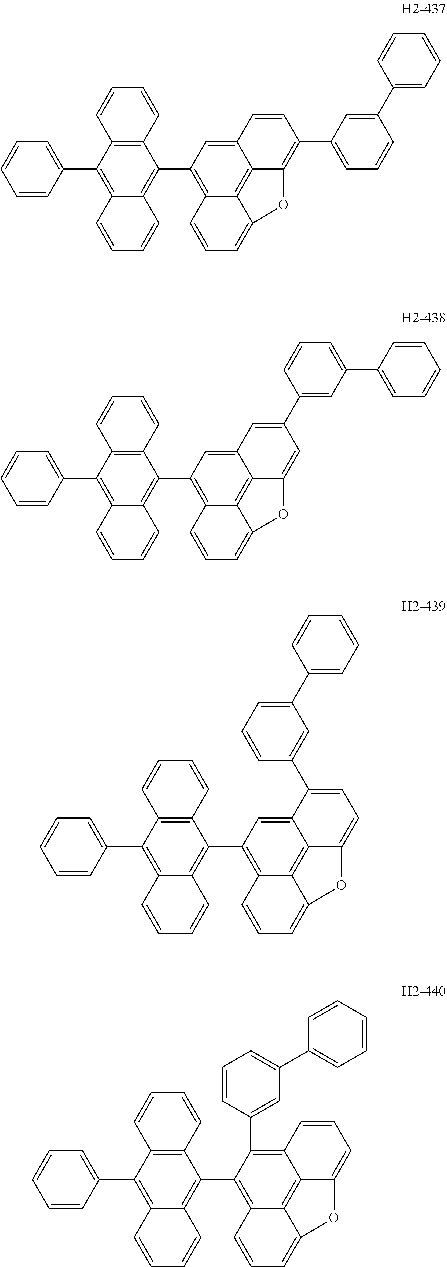 [SMILES: c1ccc(-c2cccc(-c3ccc4cc(-c5c6ccccc6c(-c6ccccc6)c6ccccc56)c5cccc6oc3c4c65)c2)cc1 ; c1ccc(-c2cccc(-c3cc4cc(-c5c6ccccc6c(-c6ccccc6)c6ccccc56)c5cccc6oc(c3)c4c65)c2)cc1 ; c1ccc(-c2cccc(-c3ccc4oc5cccc6c(-c7c8ccccc8c(-c8ccccc8)c8ccccc78)cc3c4c56)c2)cc1 ; c1ccc(-c2cccc(-c3c(-c4c5ccccc5c(-c5ccccc5)c5ccccc45)c4cccc5oc6cccc3c6c54)c2)cc1]

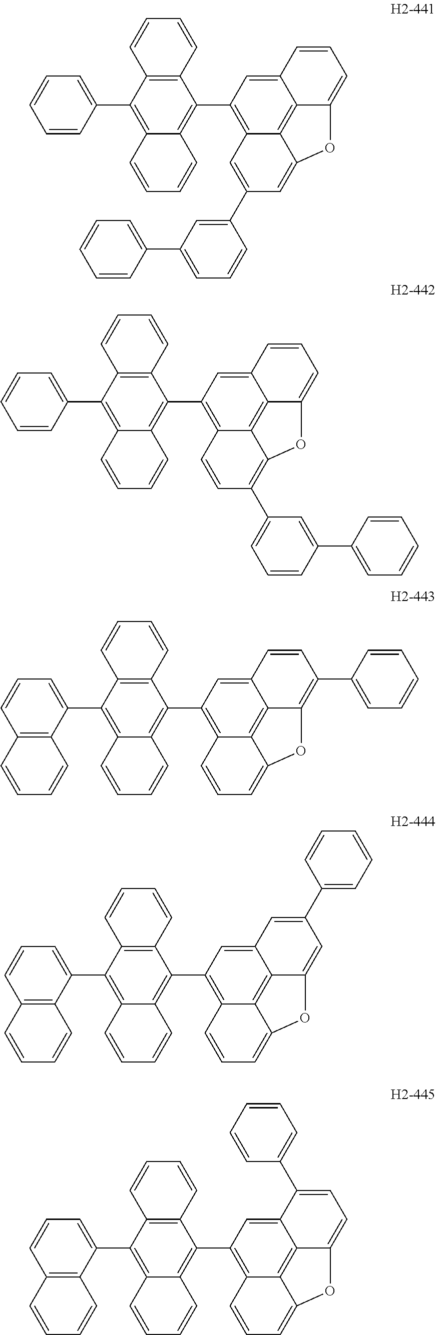

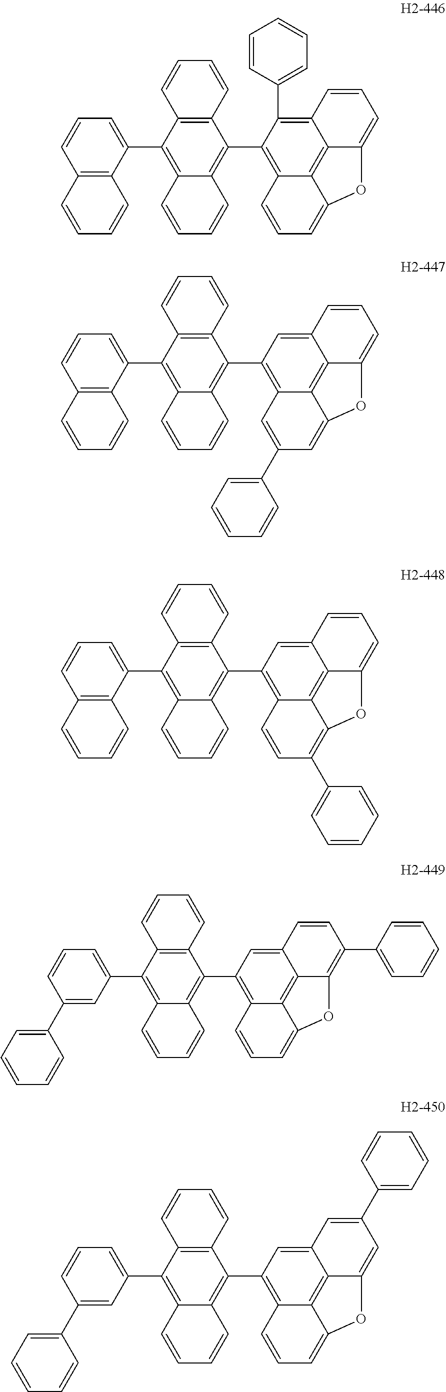

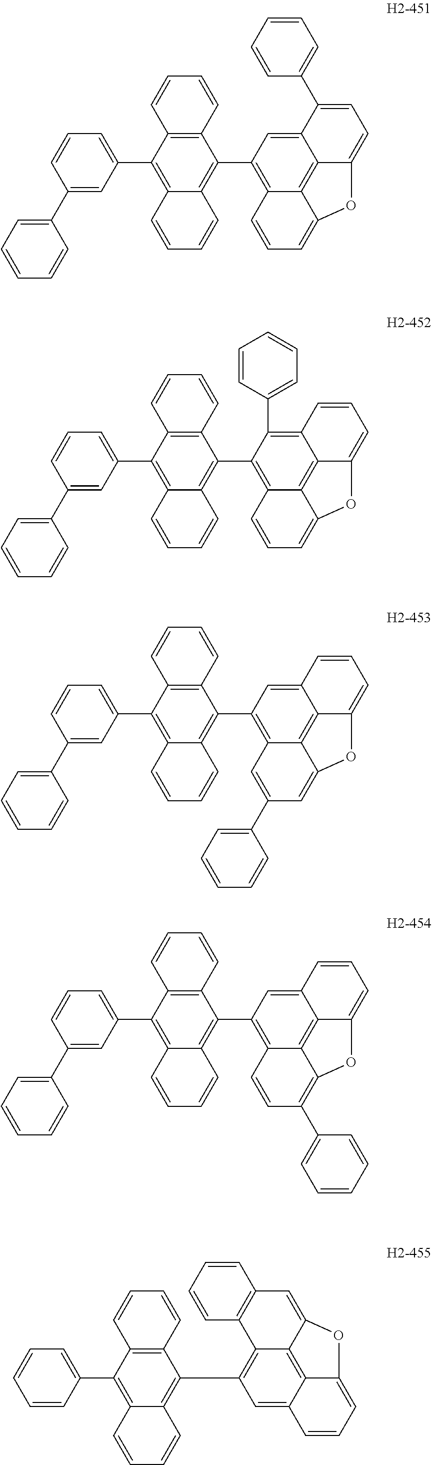 [SMILES: c1ccc(-c2cccc(-c3c4ccccc4c(-c4cc5c(-c6ccccc6)ccc6oc7cccc4c7c65)c4ccccc34)c2)cc1 ; c1ccc(-c2cccc(-c3c4ccccc4c(-c4c(-c5ccccc5)c5cccc6oc7cccc4c7c65)c4ccccc34)c2)cc1 ; c1ccc(-c2cccc(-c3c4ccccc4c(-c4cc5cccc6oc7cc(-c8ccccc8)cc4c7c56)c4ccccc34)c2)cc1 ; c1ccc(-c2cccc(-c3c4ccccc4c(-c4cc5cccc6oc7c(-c8ccccc8)ccc4c7c56)c4ccccc34)c2)cc1 ; c1ccc(-c2c3ccccc3c(-c3cc4cccc5oc6cc7ccccc7c3c6c45)c3ccccc23)cc1]

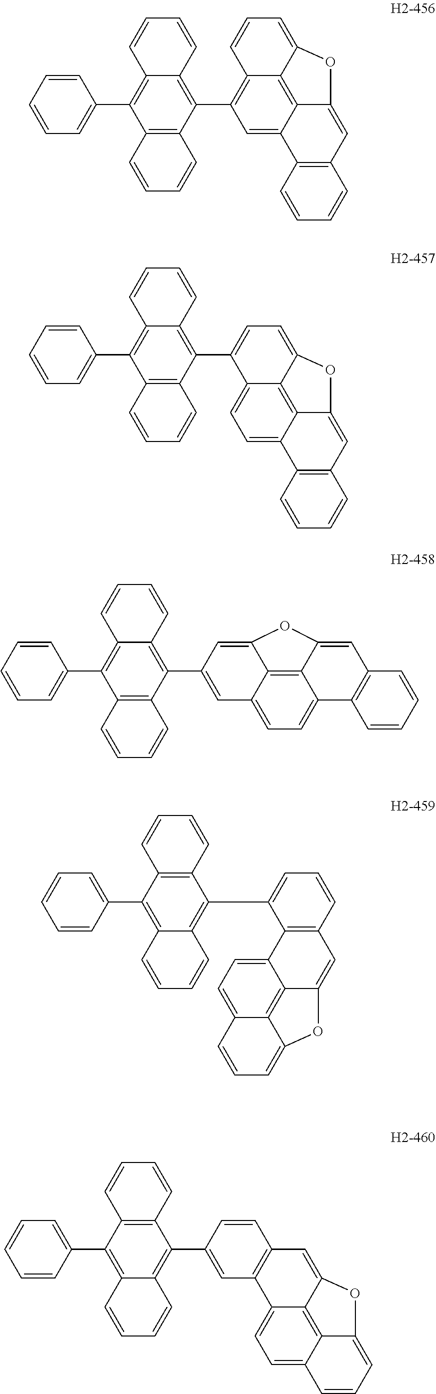 [SMILES: c1ccc(-c2c3ccccc3c(-c3cc4c5ccccc5cc5oc6cccc3c6c54)c3ccccc23)cc1 ; c1ccc(-c2c3ccccc3c(-c3ccc4oc5cc6ccccc6c6ccc3c4c56)c3ccccc23)cc1 ; c1ccc(-c2c3ccccc3c(-c3cc4ccc5c6ccccc6cc6oc(c3)c4c65)c3ccccc23)cc1 ; c1ccc(-c2c3ccccc3c(-c3cccc4cc5oc6cccc7ccc(c34)c5c76)c3ccccc23)cc1 ; c1ccc(-c2c3ccccc3c(-c3ccc4cc5oc6cccc7ccc(c4c3)c5c76)c3ccccc23)cc1]

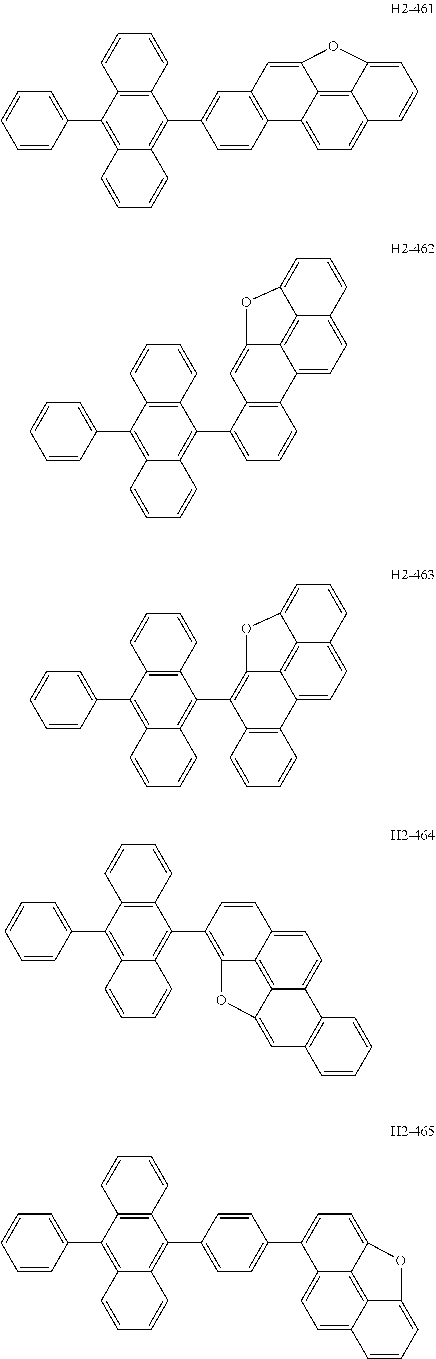 [SMILES: c1ccc(-c2c3ccccc3c(-c3ccc4c(c3)cc3oc5cccc6ccc4c3c65)c3ccccc23)cc1 ; c1ccc(-c2c3ccccc3c(-c3cccc4c3cc3oc5cccc6ccc4c3c65)c3ccccc23)cc1 ; c1ccc(-c2c3ccccc3c(-c3c4ccccc4c4ccc5cccc6oc3c4c56)c3ccccc23)cc1 ; c1ccc(-c2c3ccccc3c(-c3ccc4ccc5c6ccccc6cc6oc3c4c65)c3ccccc23)cc1 ; c1ccc(-c2c3ccccc3c(-c3ccc(-c4ccc5oc6cccc7ccc4c5c76)cc3)c3ccccc23)cc1]

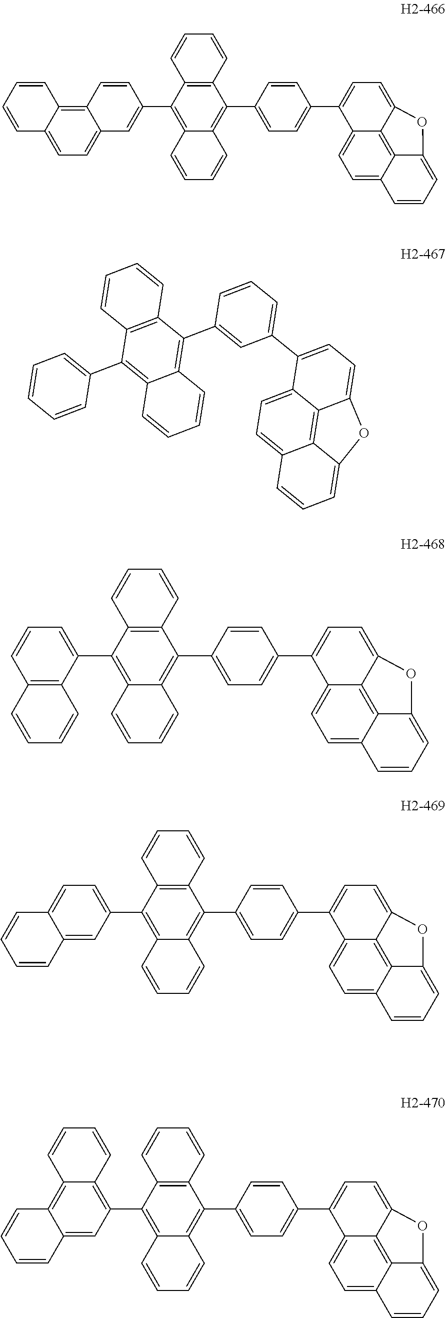 [SMILES: c1ccc2c(c1)ccc1cc(-c3c4ccccc4c(-c4ccc(-c5ccc6oc7cccc8ccc5c6c87)cc4)c4ccccc34)ccc12 ; c1ccc(-c2c3ccccc3c(-c3cccc(-c4ccc5oc6cccc7ccc4c5c76)c3)c3ccccc23)cc1 ; c1ccc2c(-c3c4ccccc4c(-c4ccc(-c5ccc6oc7cccc8ccc5c6c87)cc4)c4ccccc34)cccc2c1 ; c1ccc2cc(-c3c4ccccc4c(-c4ccc(-c5ccc6oc7cccc8ccc5c6c87)cc4)c4ccccc34)ccc2c1 ; c1ccc2c(c1)cc(-c1c3ccccc3c(-c3ccc(-c4ccc5oc6cccc7ccc4c5c76)cc3)c3ccccc13)c1ccccc12]

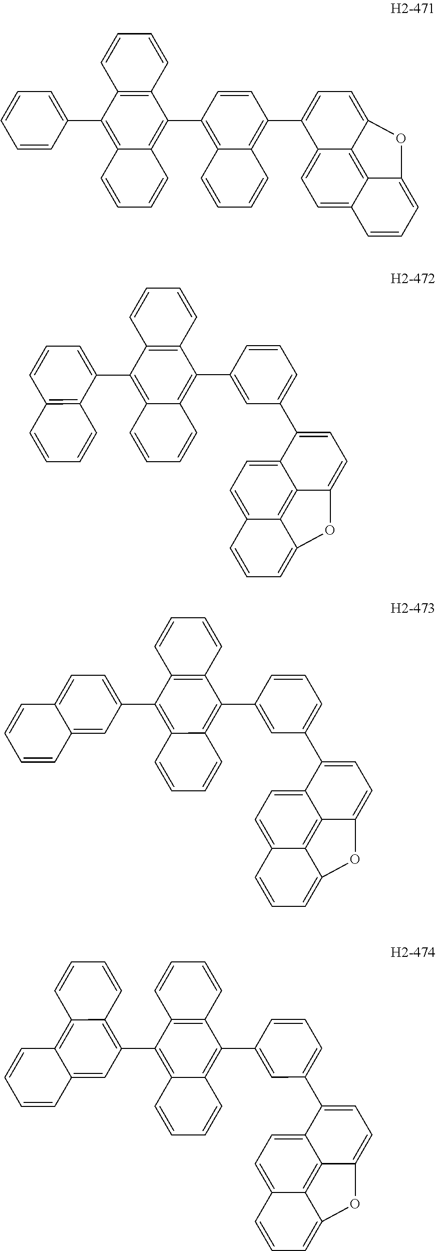 [SMILES: c1ccc(-c2c3ccccc3c(-c3ccc(-c4ccc5oc6cccc7ccc4c5c76)c4ccccc34)c3ccccc23)cc1 ; c1cc(-c2c3ccccc3c(-c3cccc4ccccc34)c3ccccc23)cc(-c2ccc3oc4cccc5ccc2c3c54)c1 ; c1cc(-c2c3ccccc3c(-c3ccc4ccccc4c3)c3ccccc23)cc(-c2ccc3oc4cccc5ccc2c3c54)c1 ; c1cc(-c2c3ccccc3c(-c3cc4ccccc4c4ccccc34)c3ccccc23)cc(-c2ccc3oc4cccc5ccc2c3c54)c1]

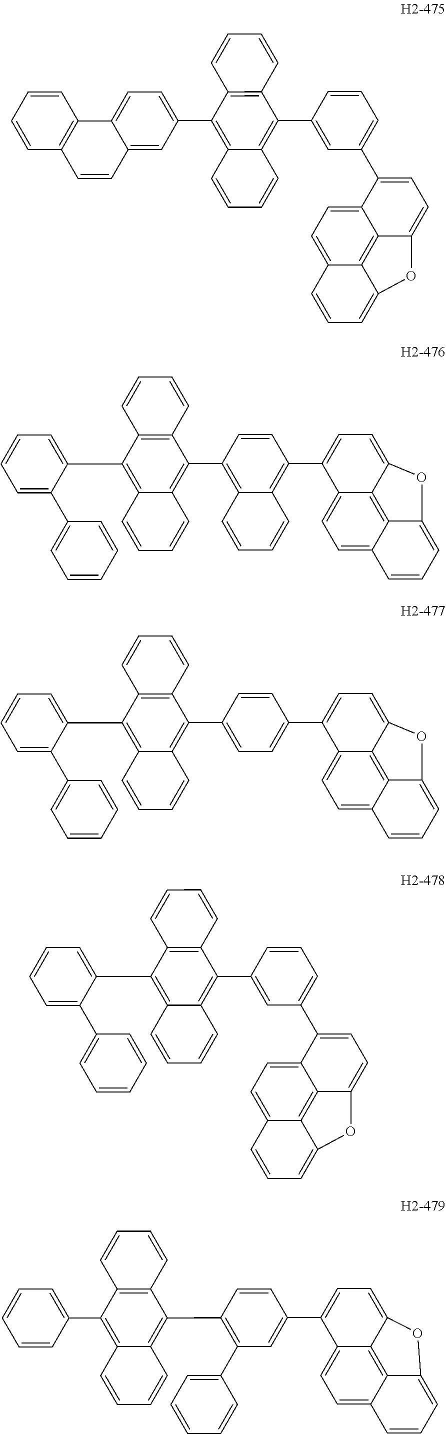 [SMILES: c1cc(-c2c3ccccc3c(-c3ccc4c(ccc5ccccc54)c3)c3ccccc23)cc(-c2ccc3oc4cccc5ccc2c3c54)c1 ; c1ccc(-c2ccccc2-c2c3ccccc3c(-c3ccc(-c4ccc5oc6cccc7ccc4c5c76)c4ccccc34)c3ccccc23)cc1 ; c1ccc(-c2ccccc2-c2c3ccccc3c(-c3ccc(-c4ccc5oc6cccc7ccc4c5c76)cc3)c3ccccc23)cc1 ; c1ccc(-c2ccccc2-c2c3ccccc3c(-c3cccc(-c4ccc5oc6cccc7ccc4c5c76)c3)c3ccccc23)cc1 ; c1ccc(-c2cc(-c3ccc4oc5cccc6ccc3c4c65)ccc2-c2c3ccccc3c(-c3ccccc3)c3ccccc23)cc1]

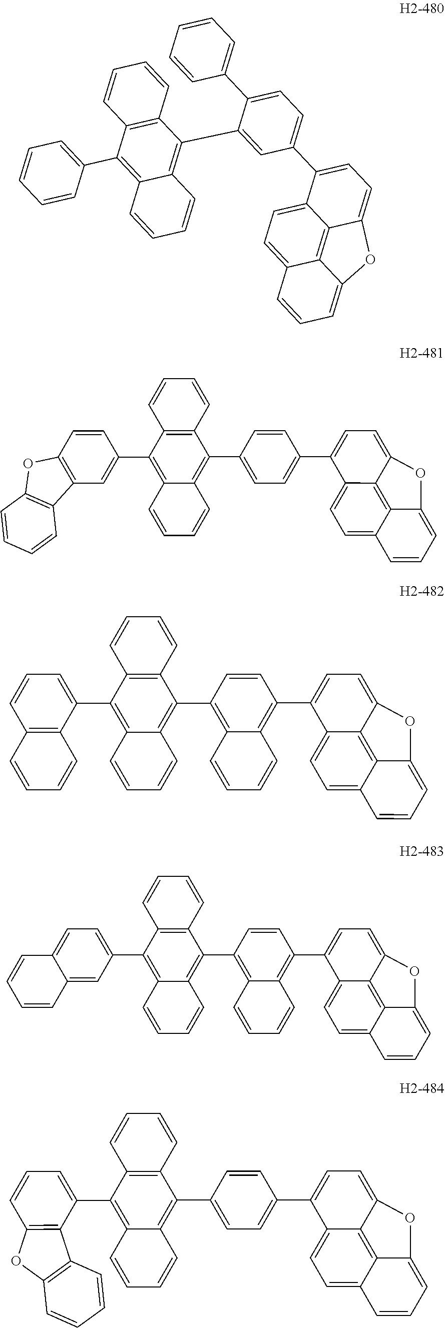 [SMILES: c1ccc(-c2ccc(-c3ccc4oc5cccc6ccc3c4c65)cc2-c2c3ccccc3c(-c3ccccc3)c3ccccc23)cc1 ; c1ccc2c(c1)oc1ccc(-c3c4ccccc4c(-c4ccc(-c5ccc6oc7cccc8ccc5c6c87)cc4)c4ccccc34)cc12 ; c1ccc2c(-c3c4ccccc4c(-c4ccc(-c5ccc6oc7cccc8ccc5c6c87)c5ccccc45)c4ccccc34)cccc2c1 ; c1ccc2cc(-c3c4ccccc4c(-c4ccc(-c5ccc6oc7cccc8ccc5c6c87)c5ccccc45)c4ccccc34)ccc2c1 ; c1ccc2c(c1)oc1cccc(-c3c4ccccc4c(-c4ccc(-c5ccc6oc7cccc8ccc5c6c87)cc4)c4ccccc34)c12]

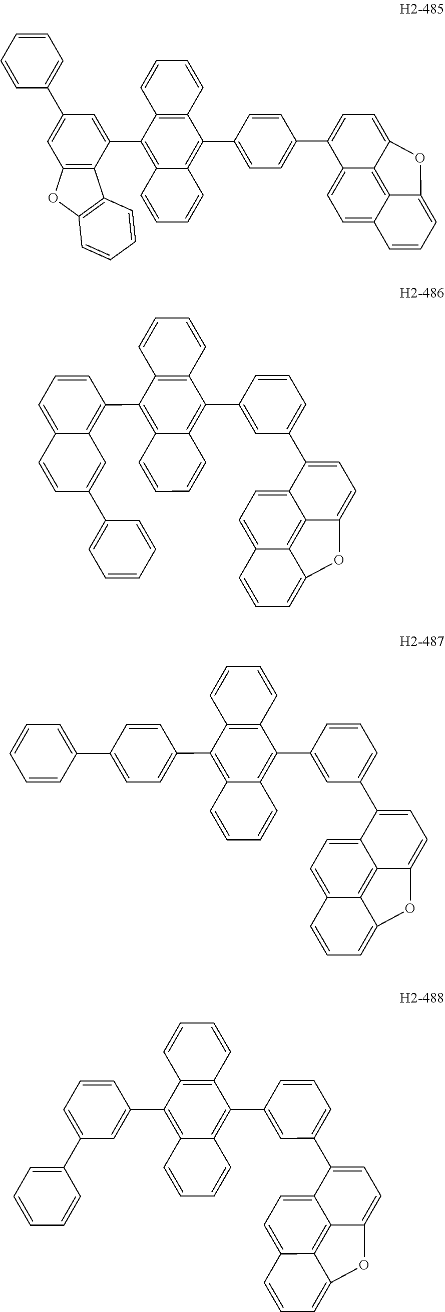 [SMILES: c1ccc(-c2cc(-c3c4ccccc4c(-c4ccc(-c5ccc6oc7cccc8ccc5c6c87)cc4)c4ccccc34)c3c(c2)oc2ccccc23)cc1 ; c1ccc(-c2ccc3cccc(-c4c5ccccc5c(-c5cccc(-c6ccc7oc8cccc9ccc6c7c98)c5)c5ccccc45)c3c2)cc1 ; c1ccc(-c2ccc(-c3c4ccccc4c(-c4cccc(-c5ccc6oc7cccc8ccc5c6c87)c4)c4ccccc34)cc2)cc1 ; c1ccc(-c2cccc(-c3c4ccccc4c(-c4cccc(-c5ccc6oc7cccc8ccc5c6c87)c4)c4ccccc34)c2)cc1]

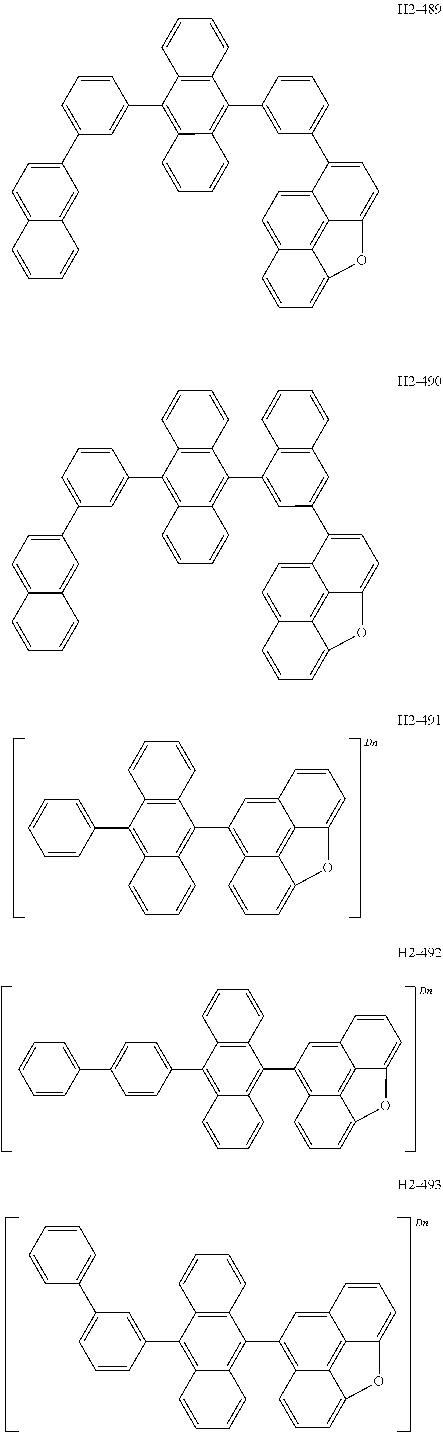

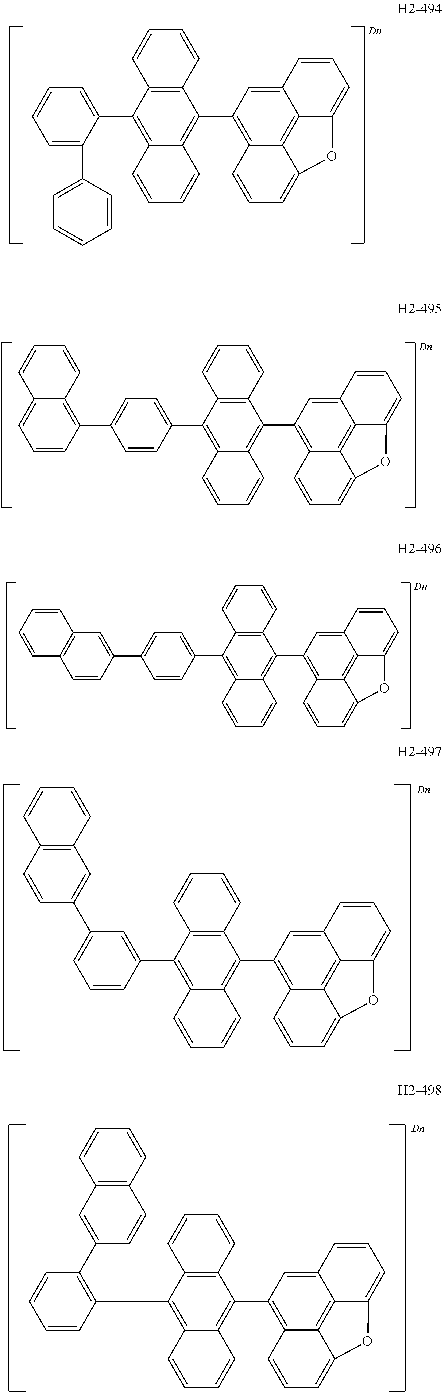

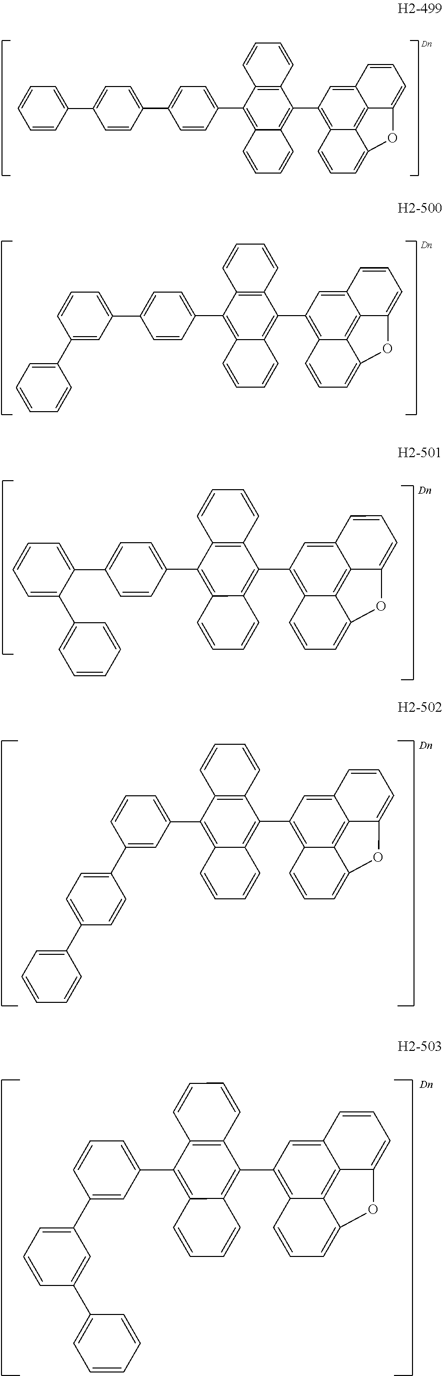

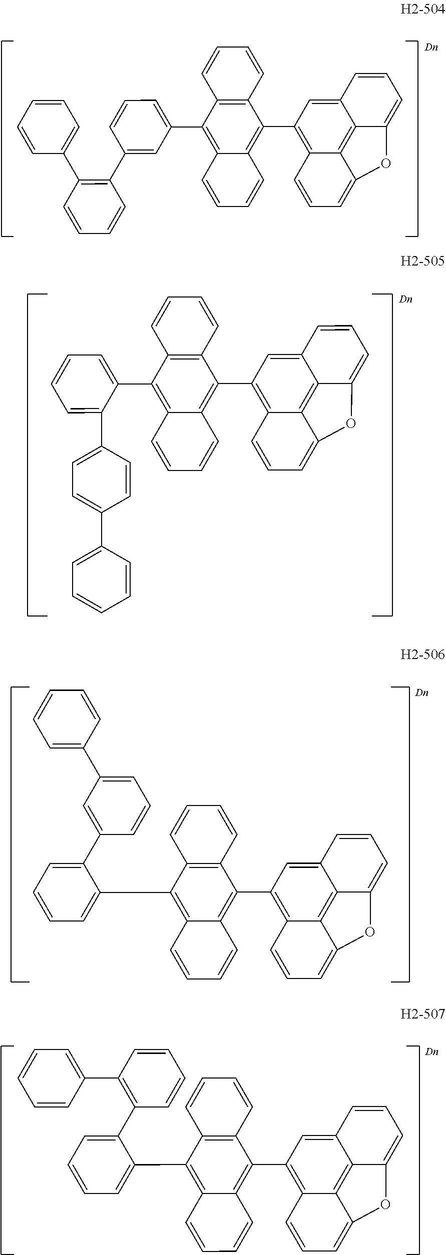

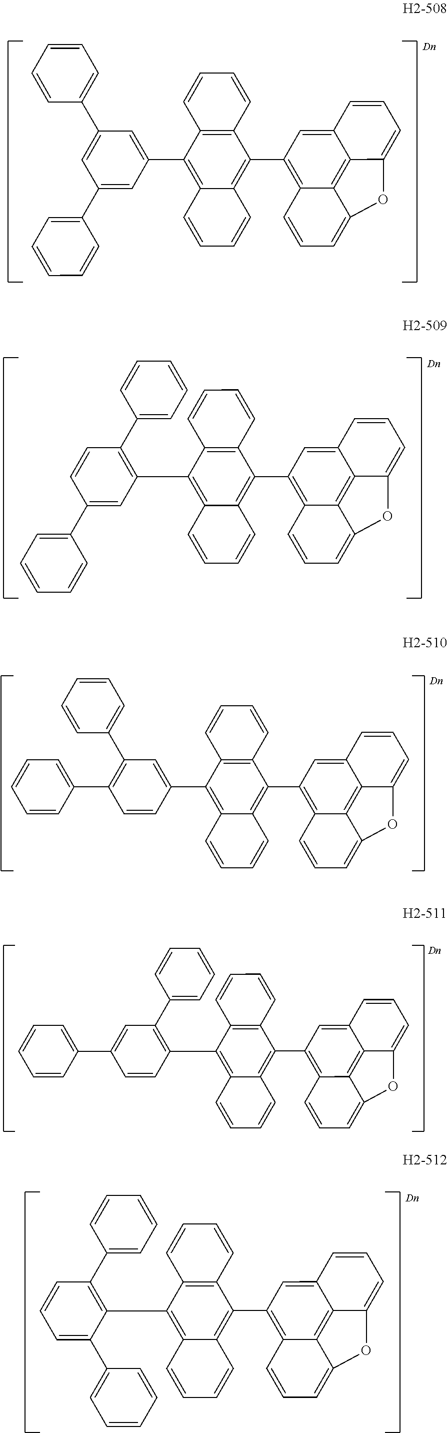

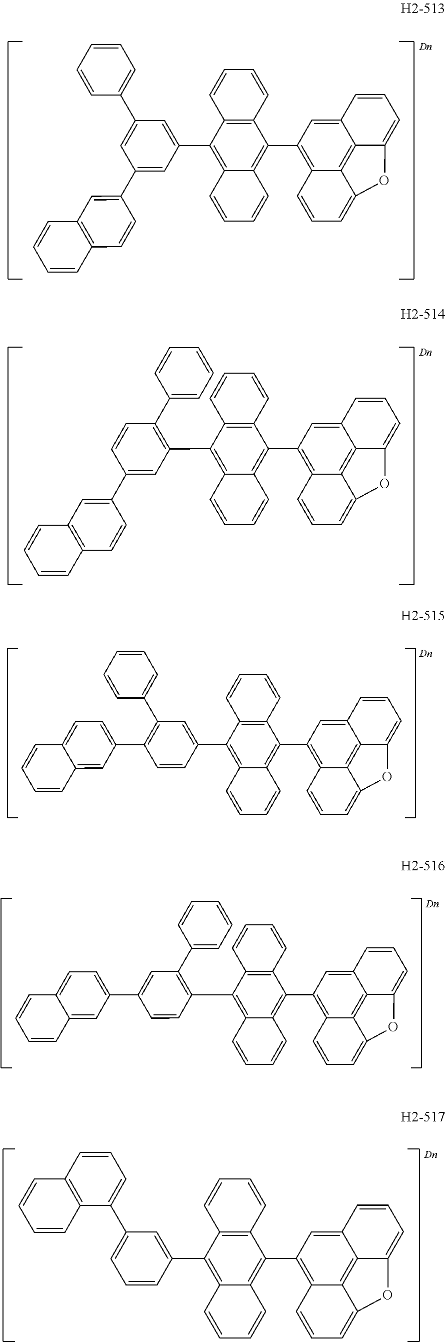

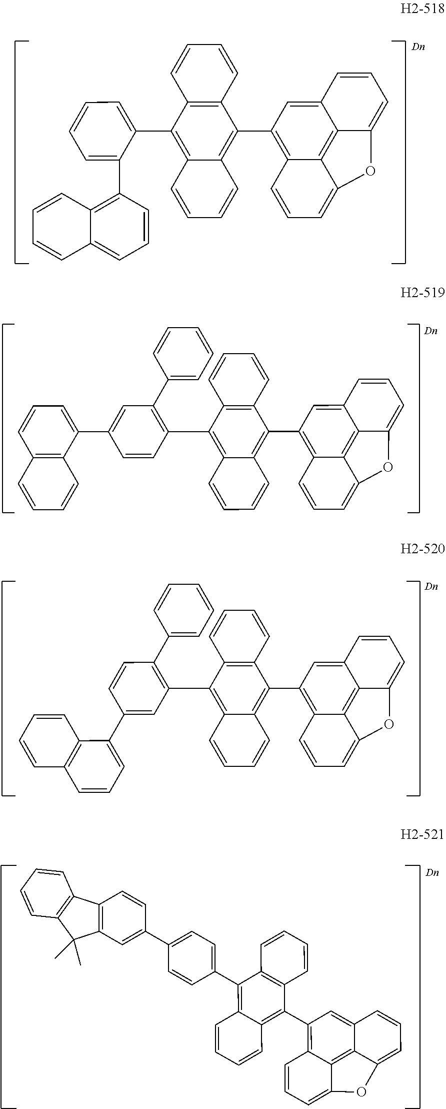

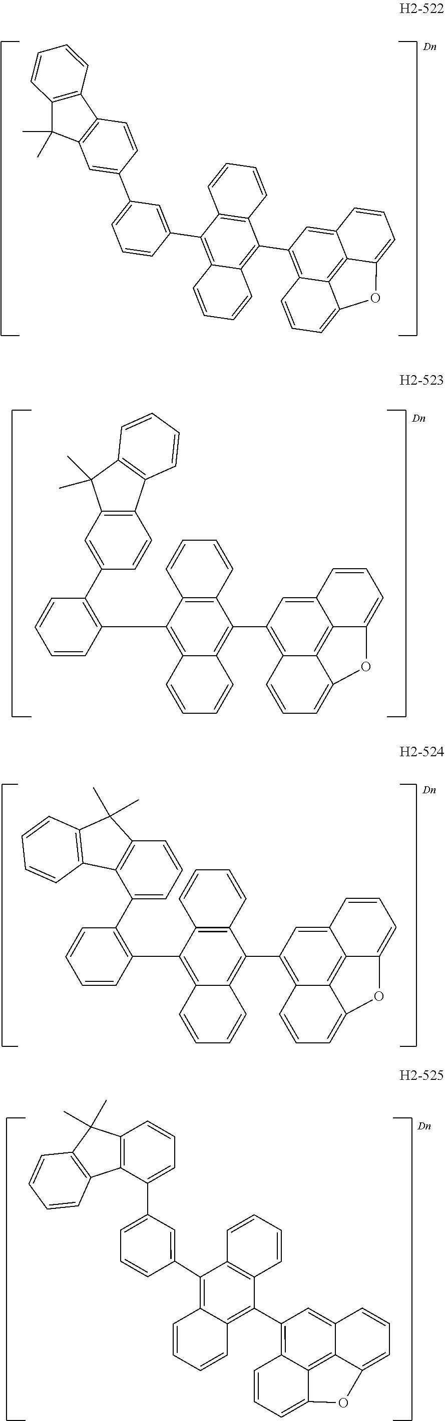

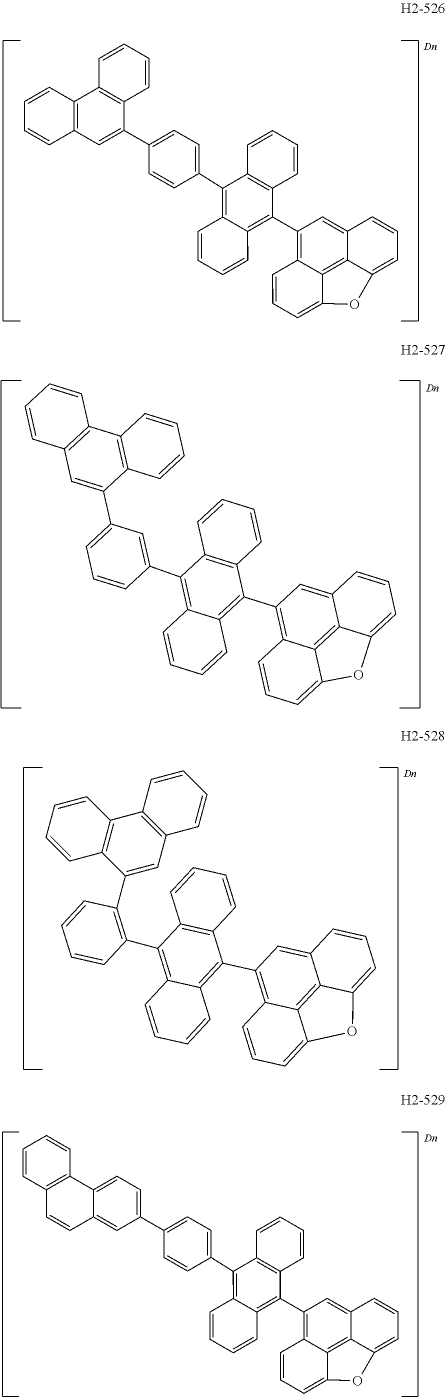

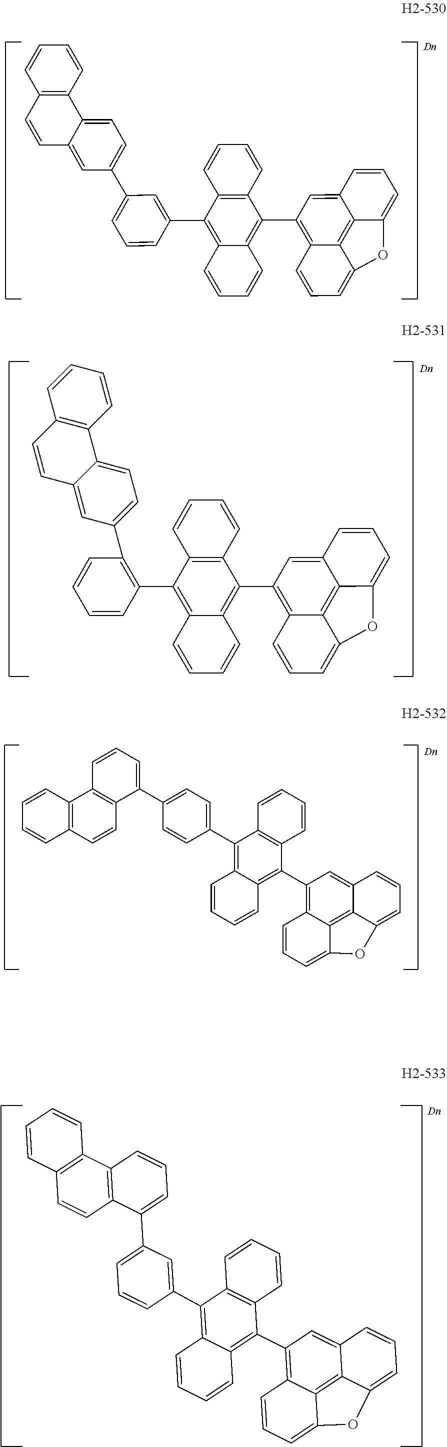

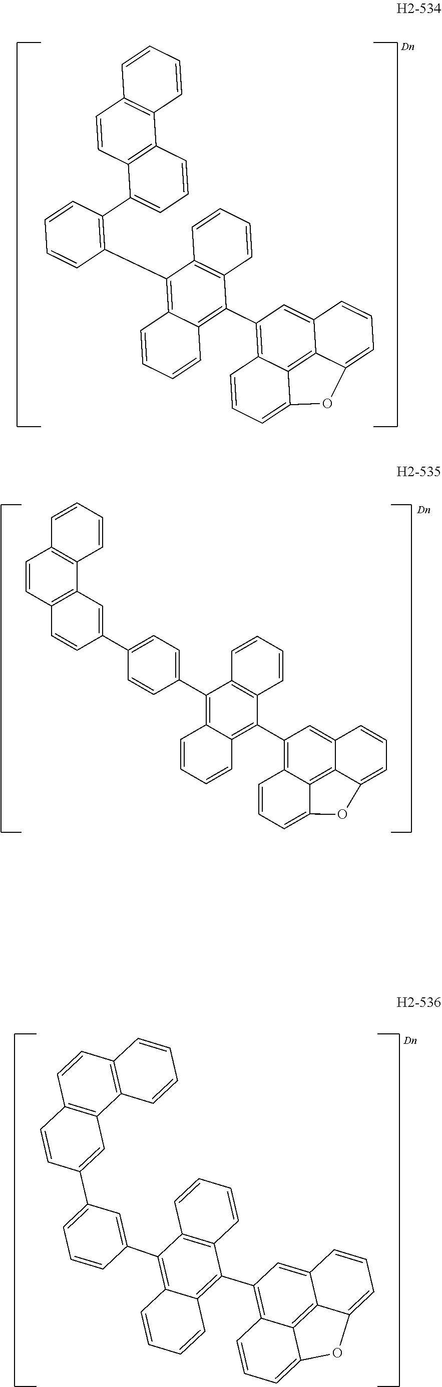

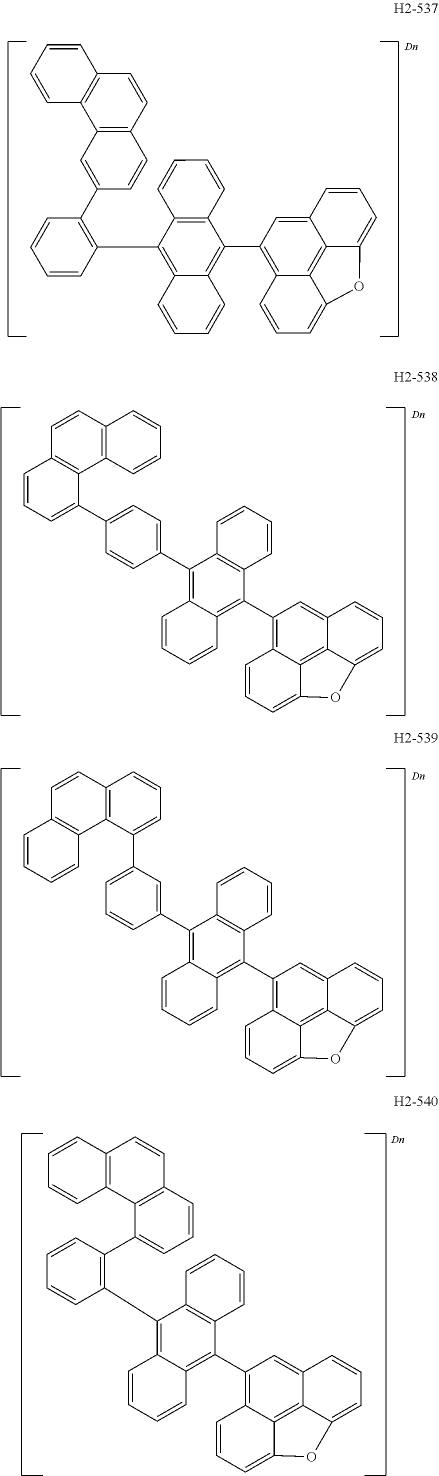

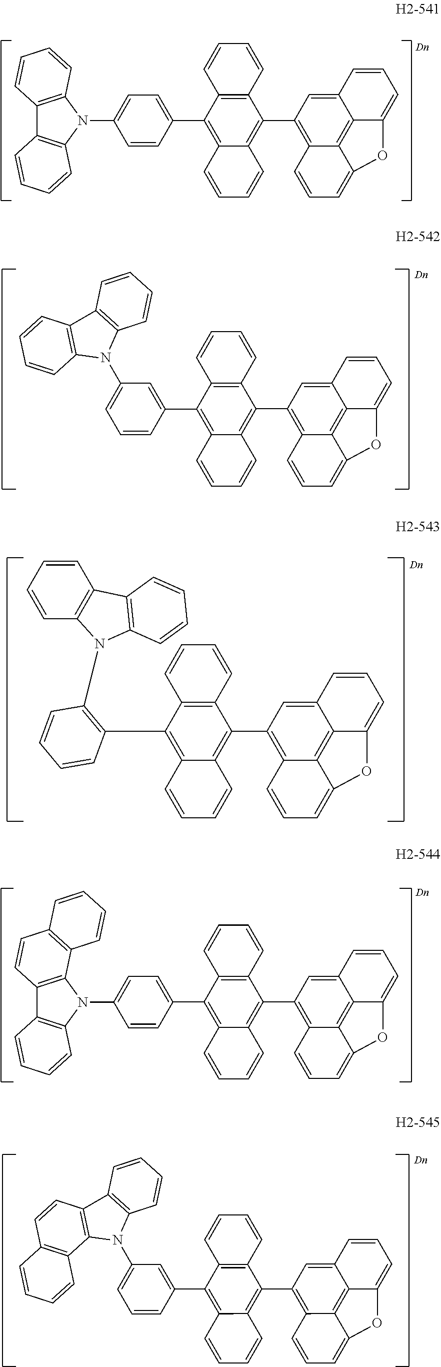

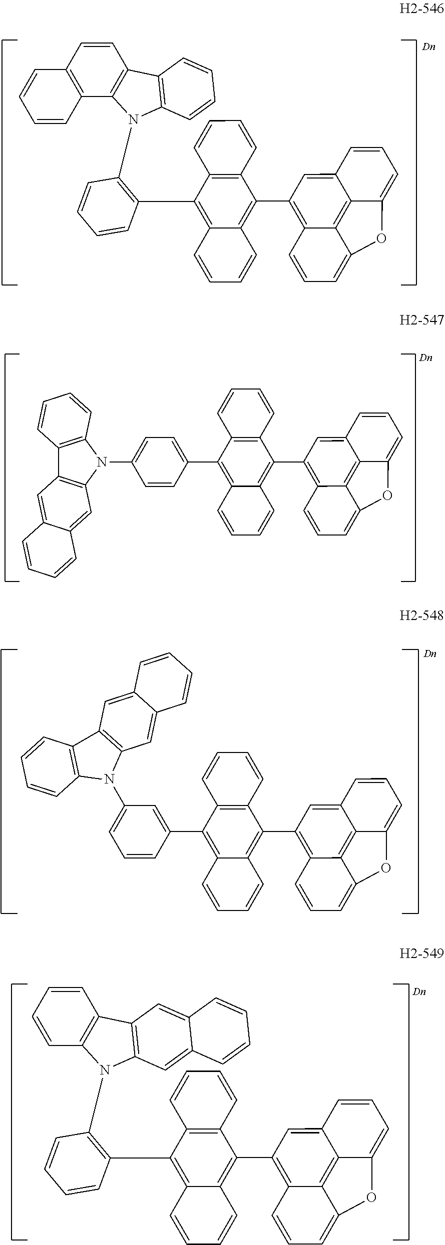

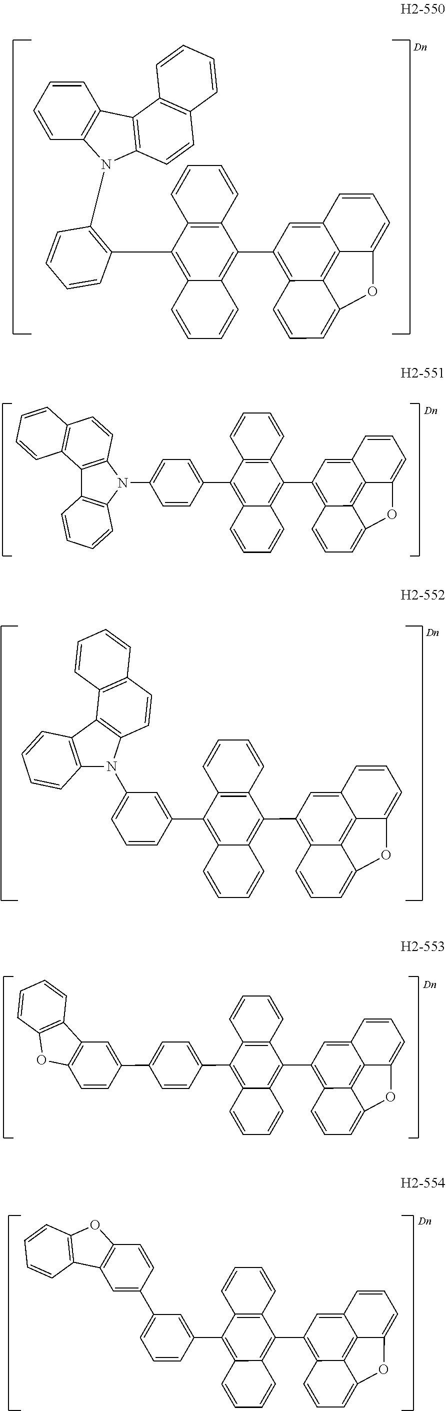

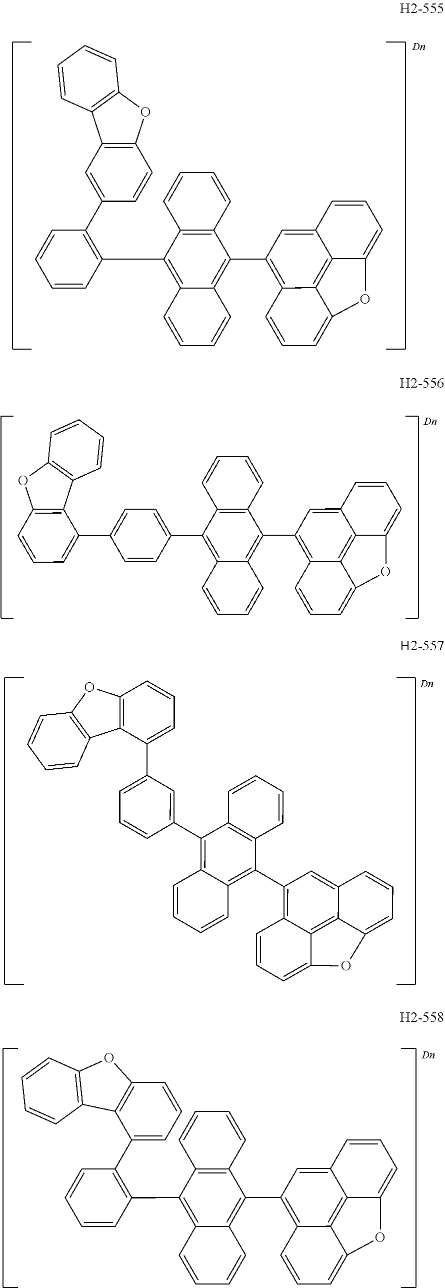

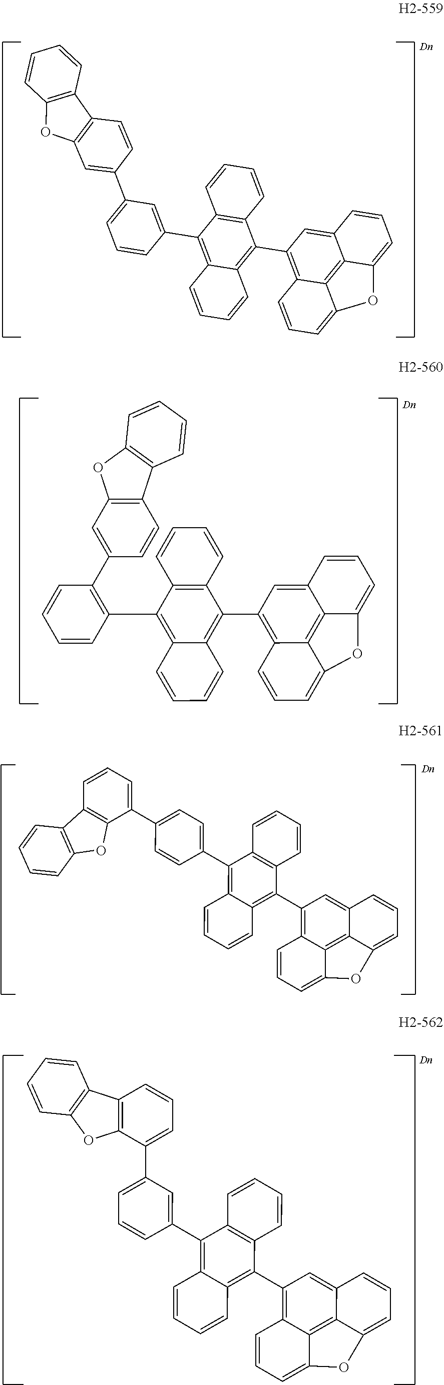

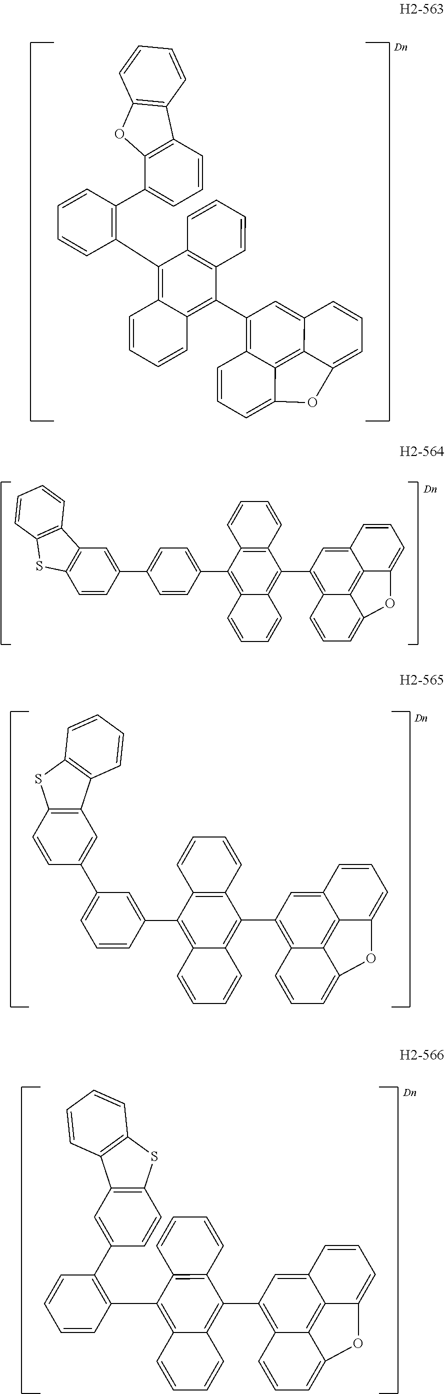

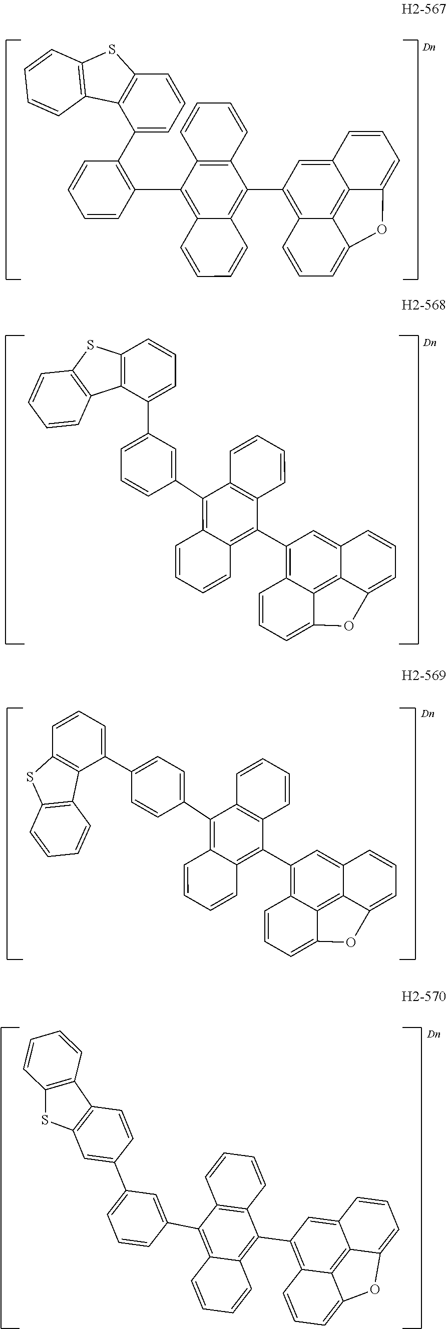

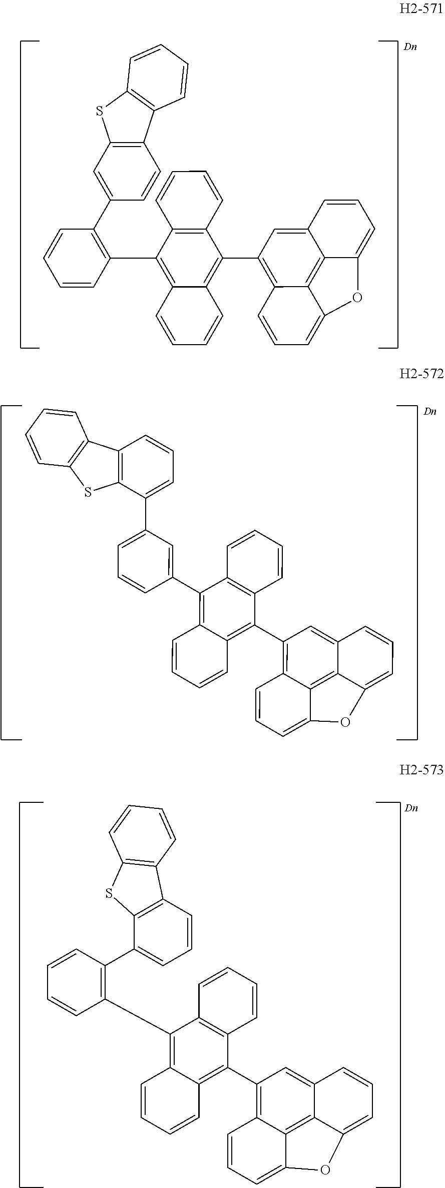

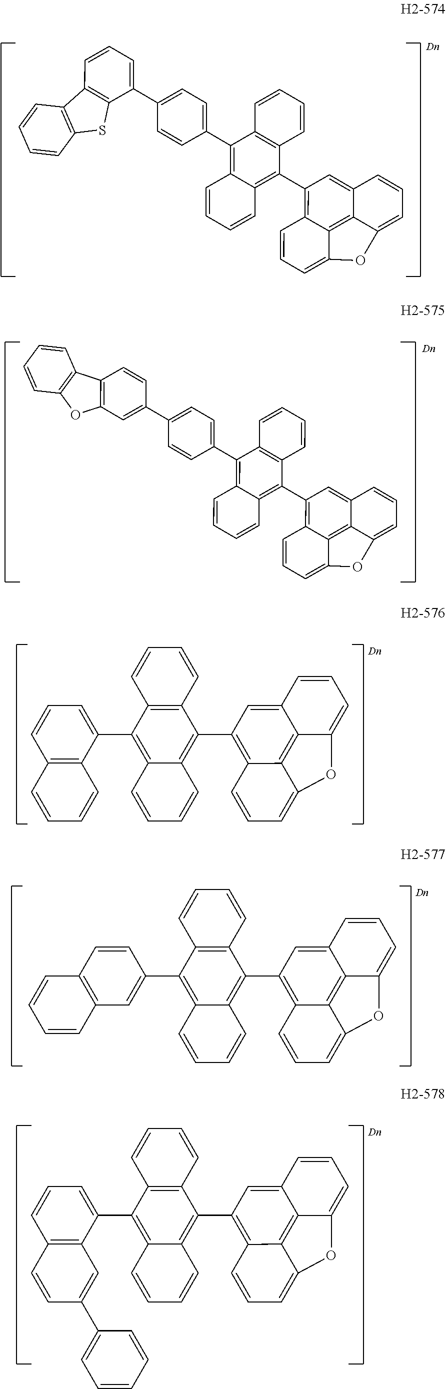

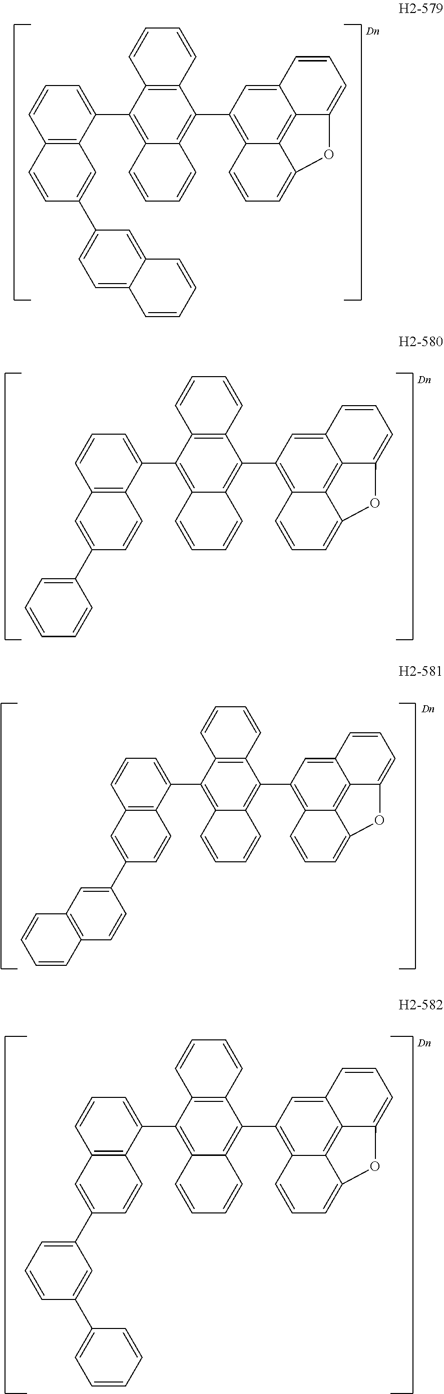

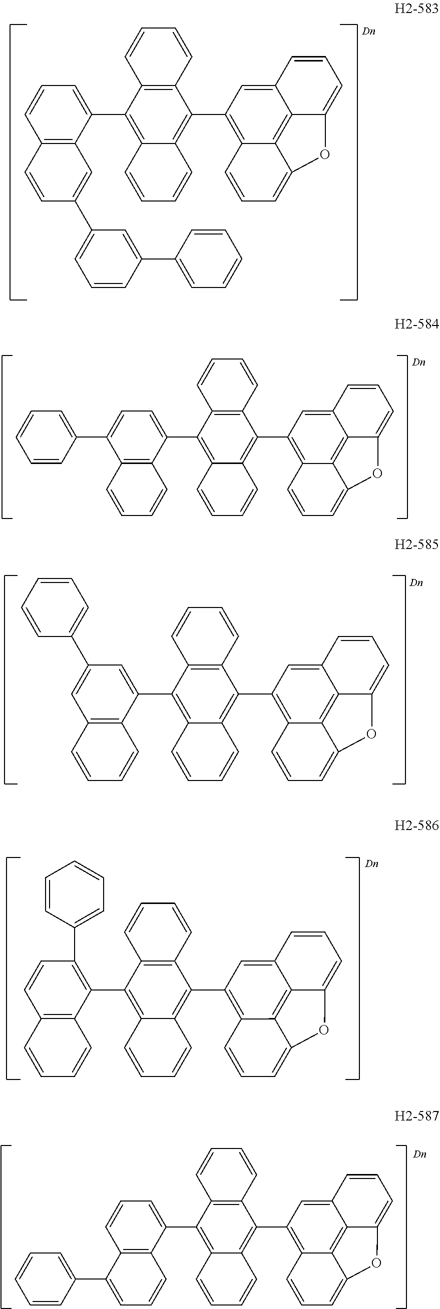

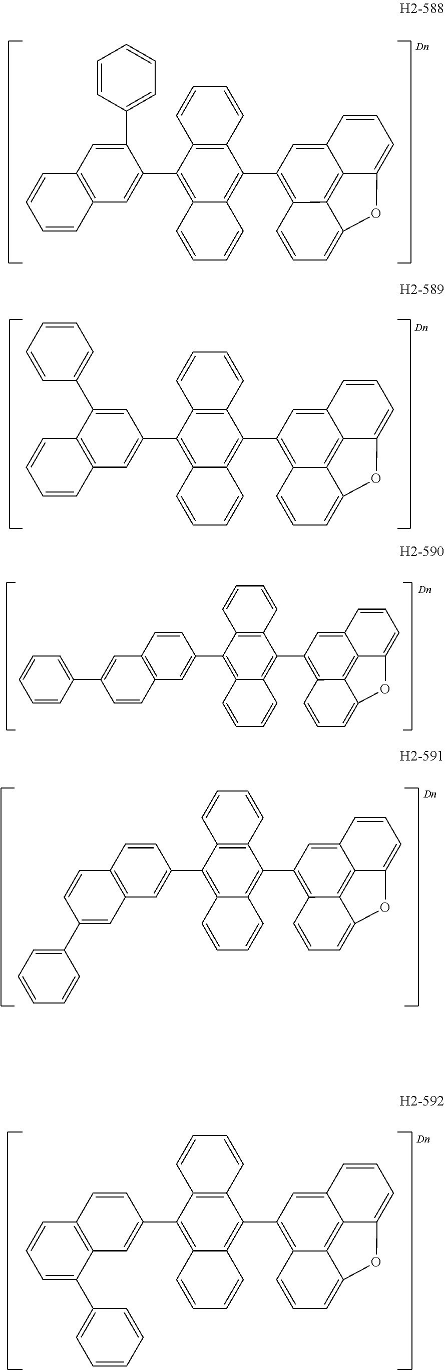

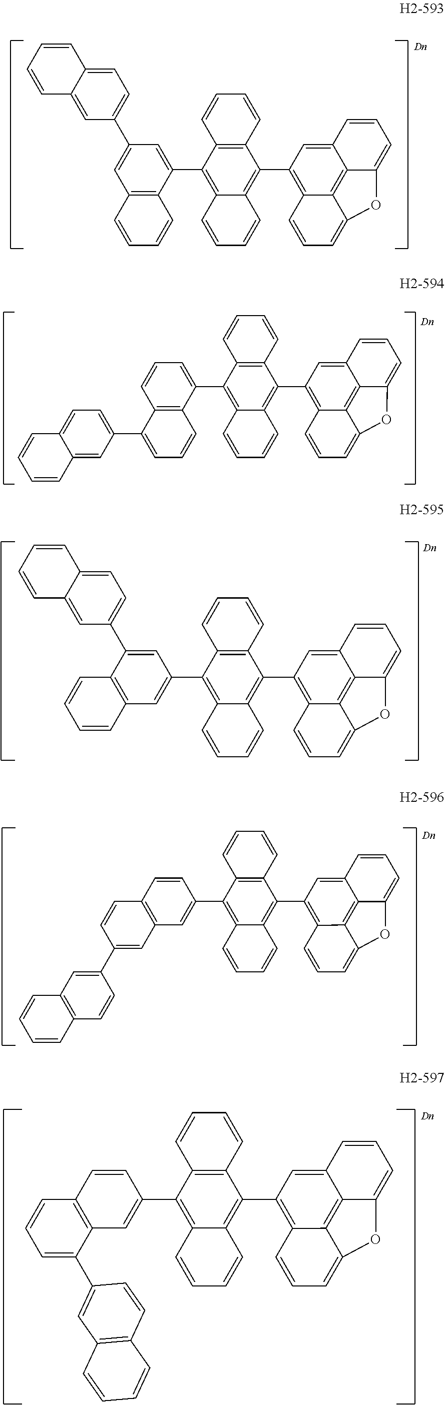

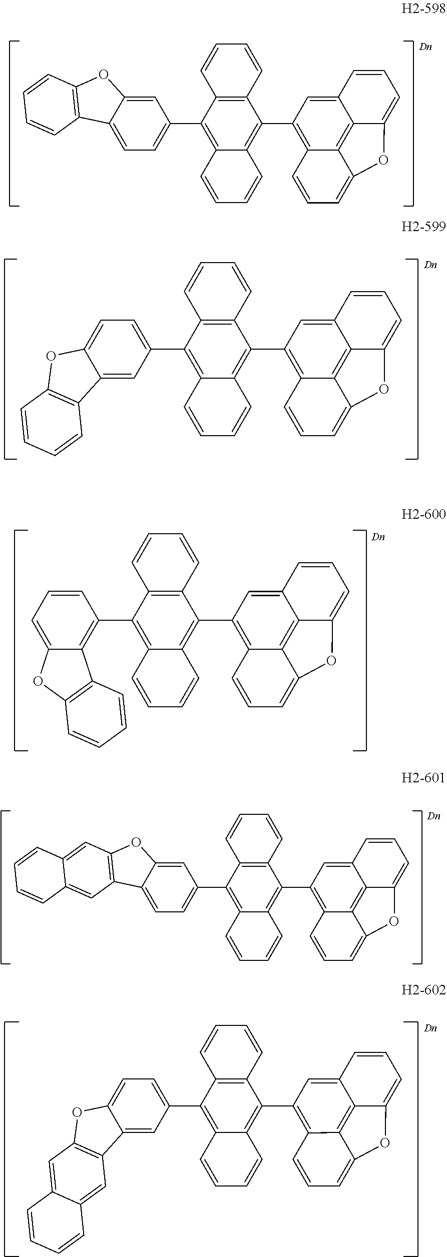

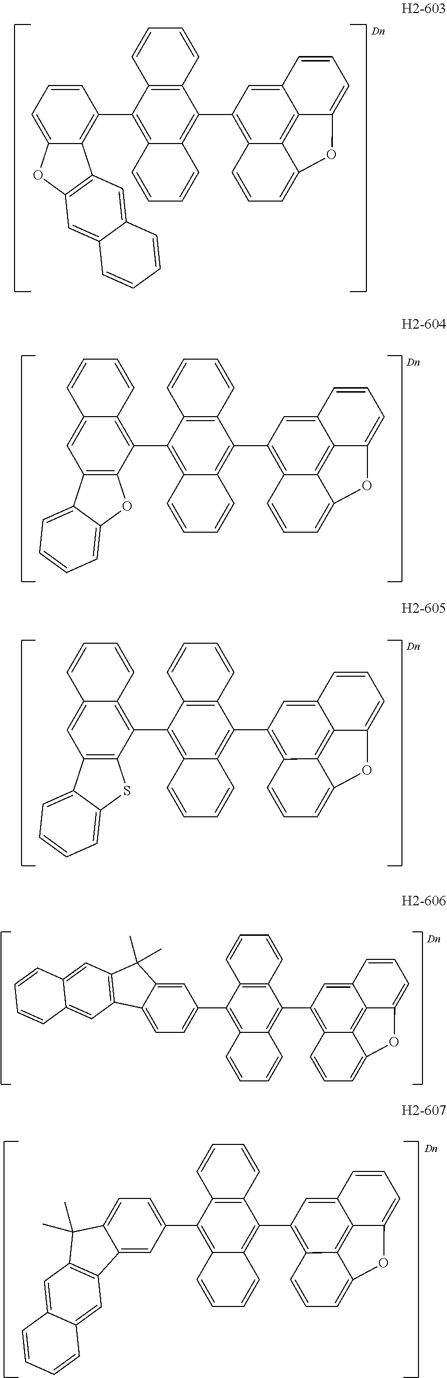

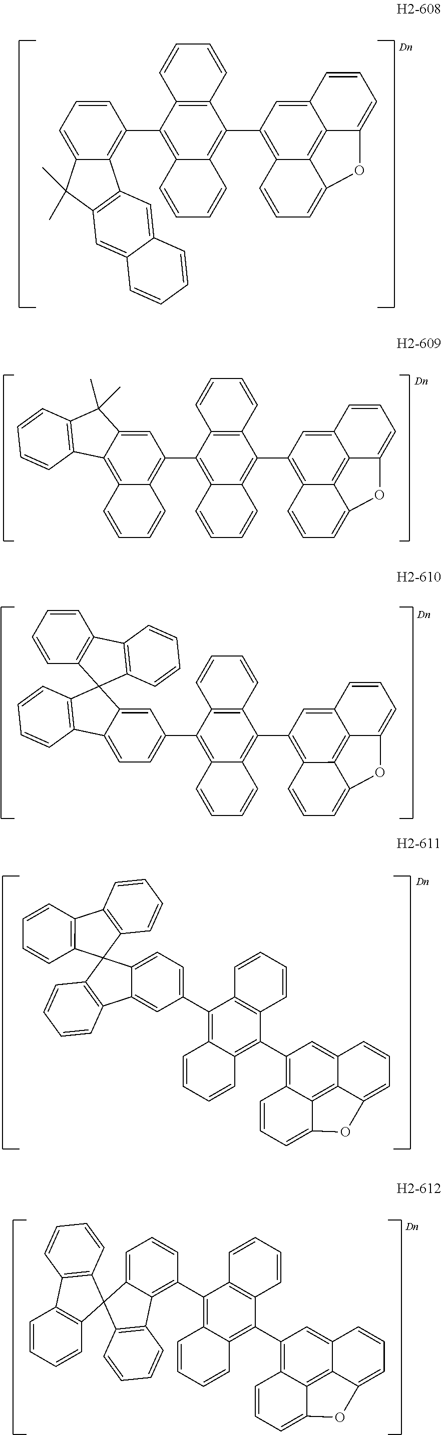

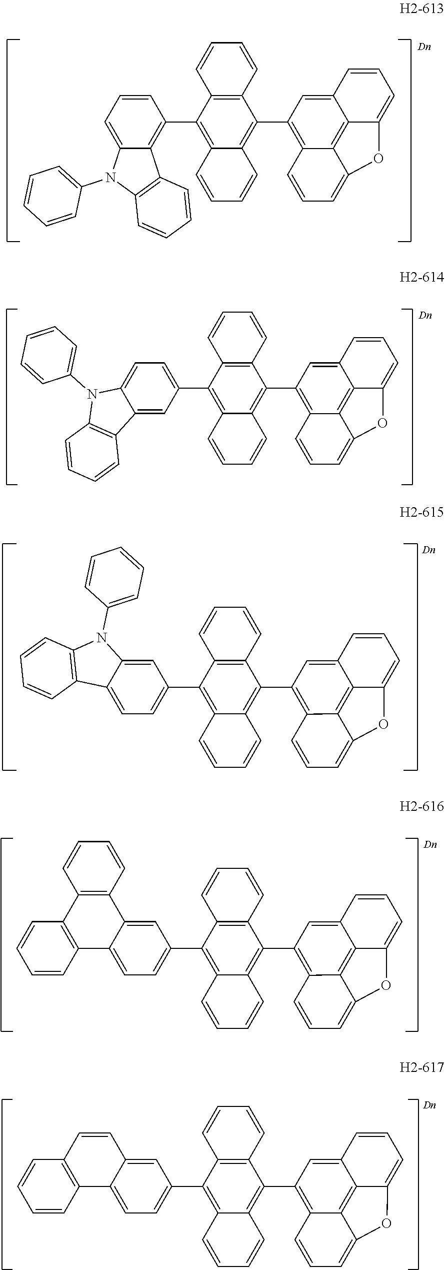

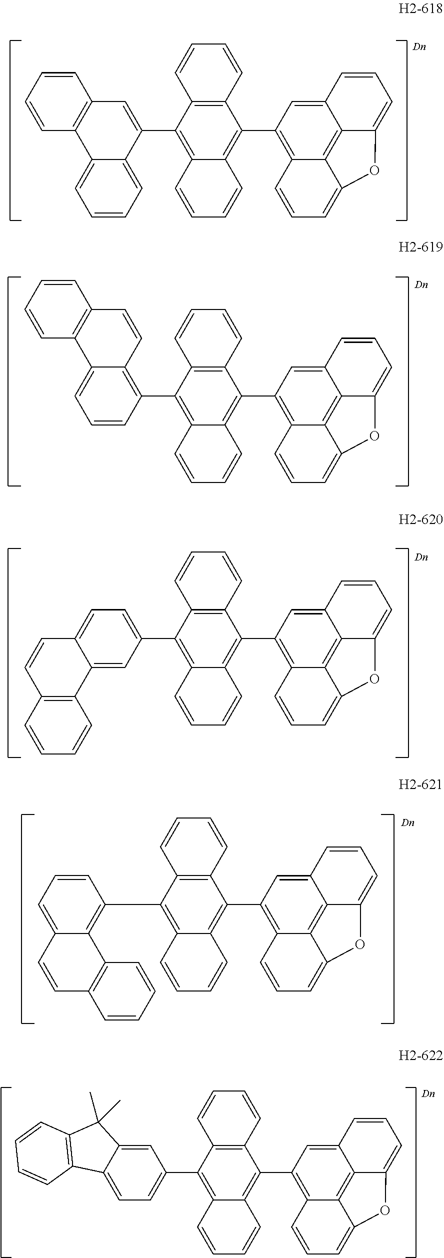

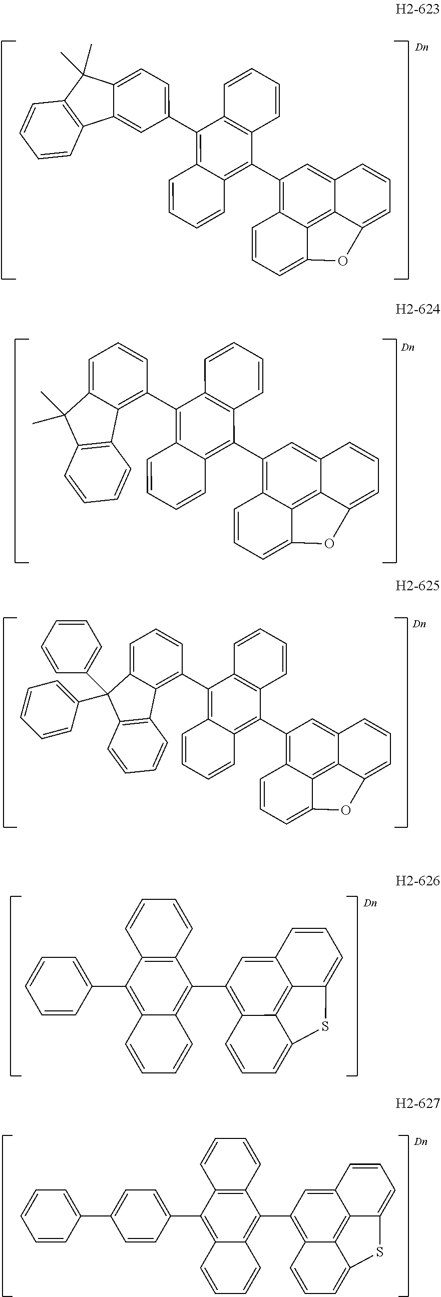

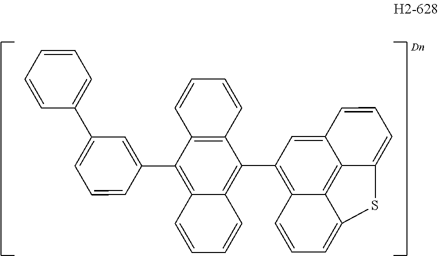

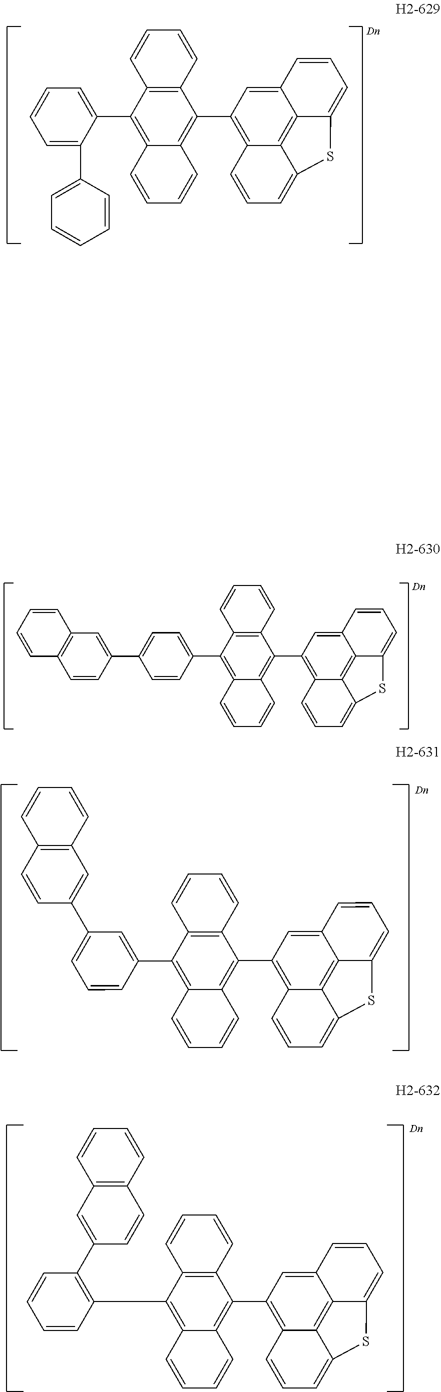

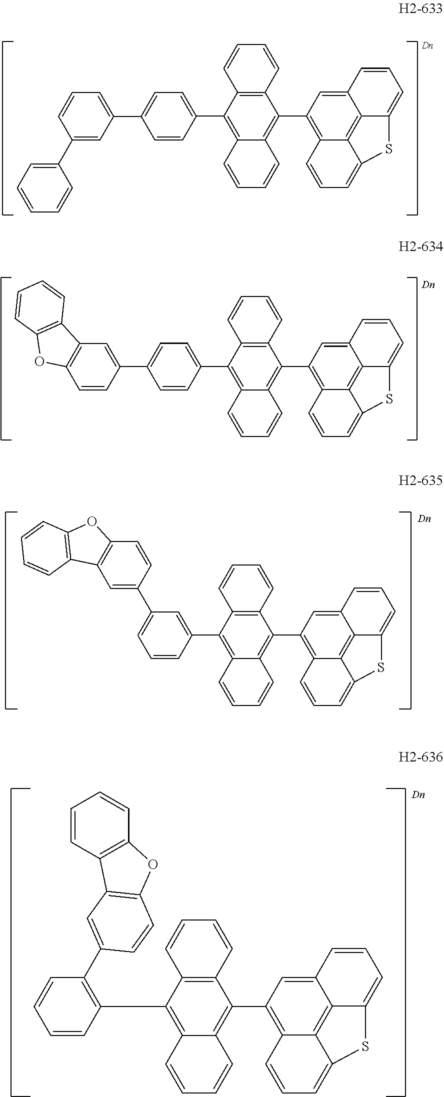

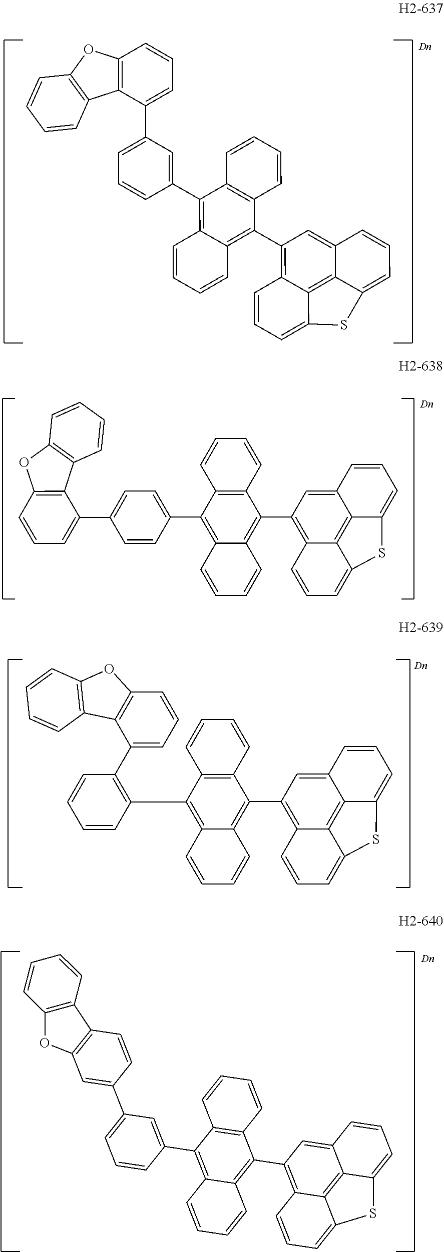

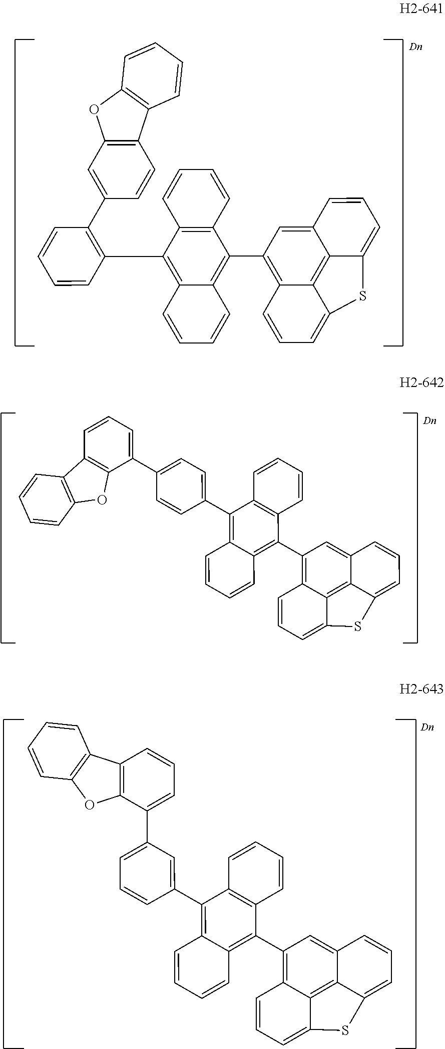

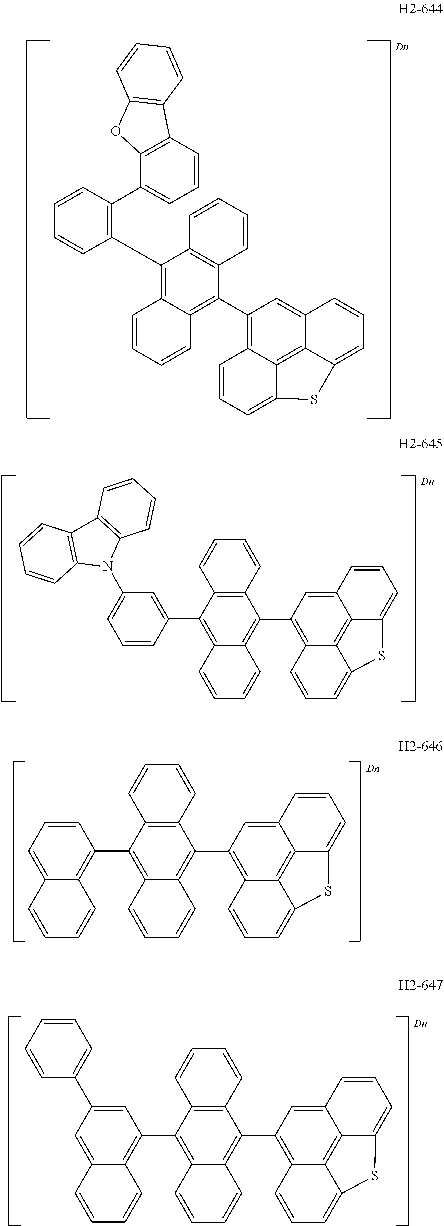

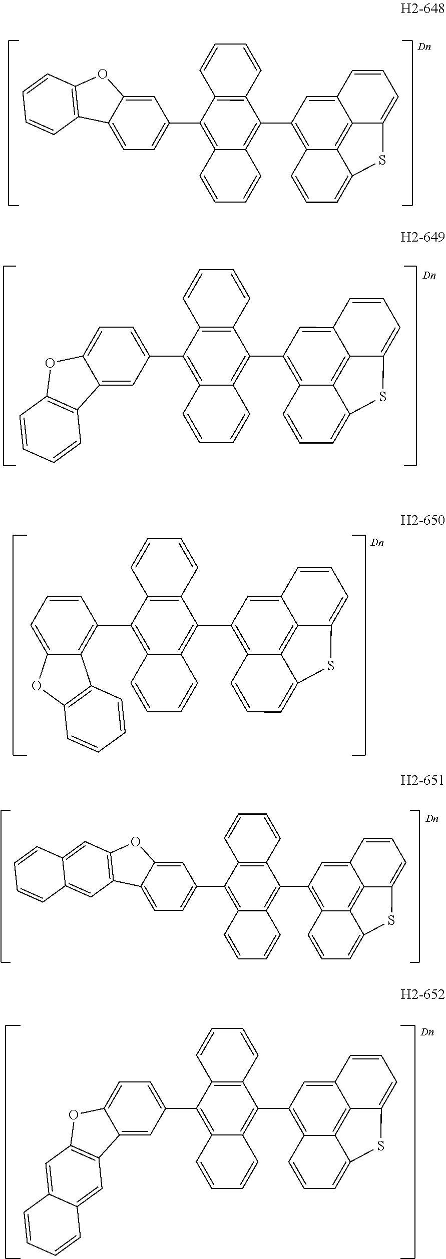

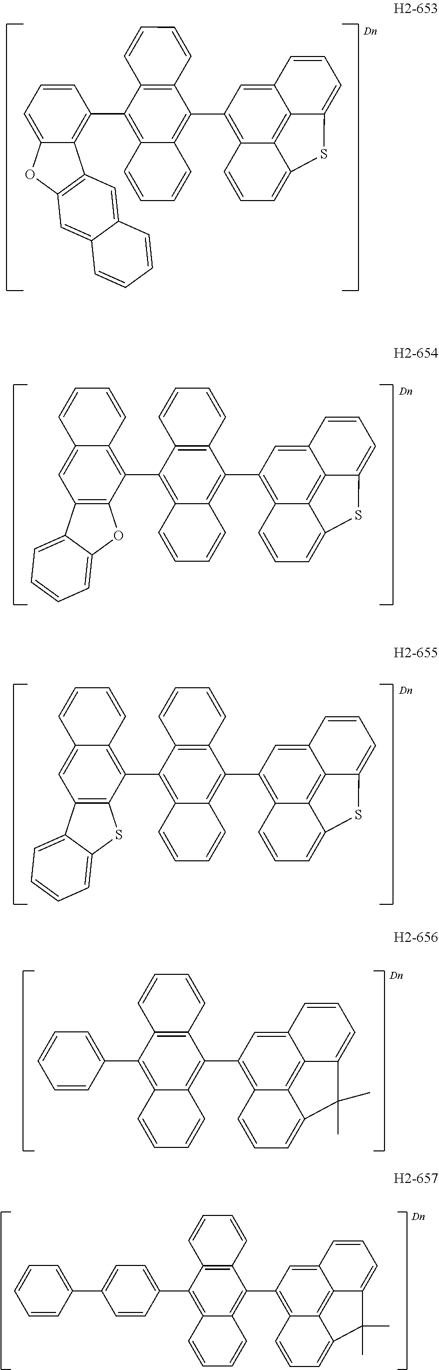

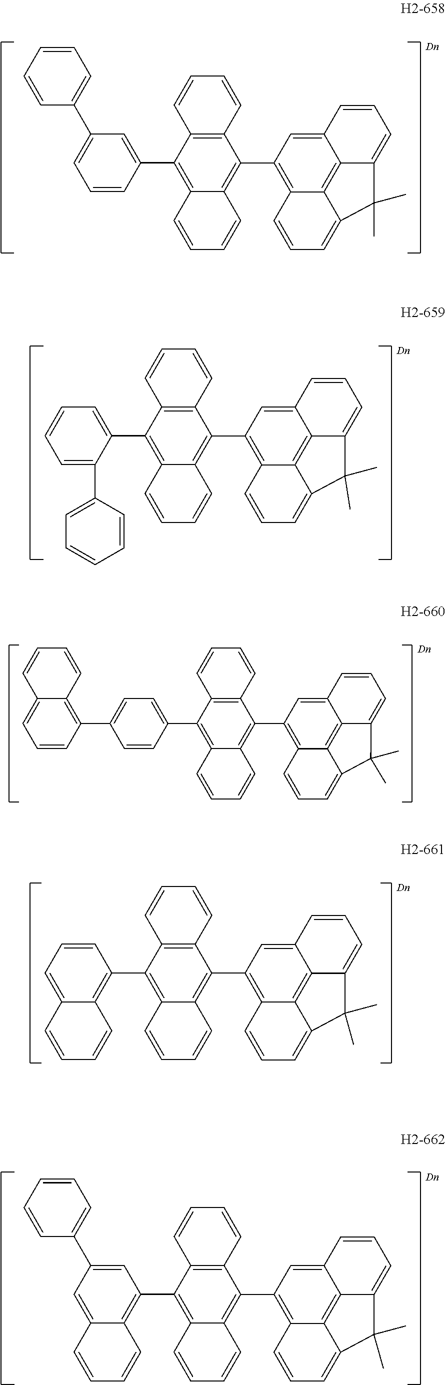

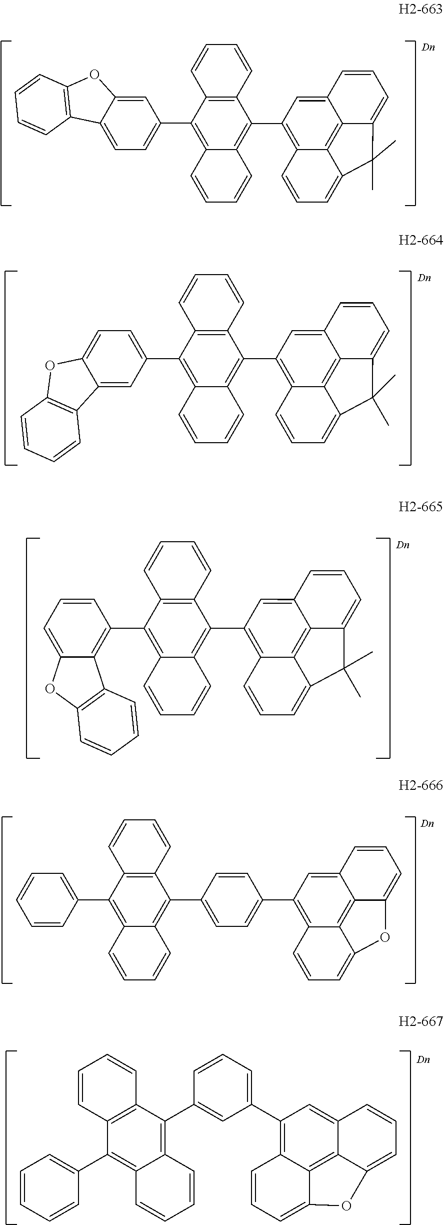

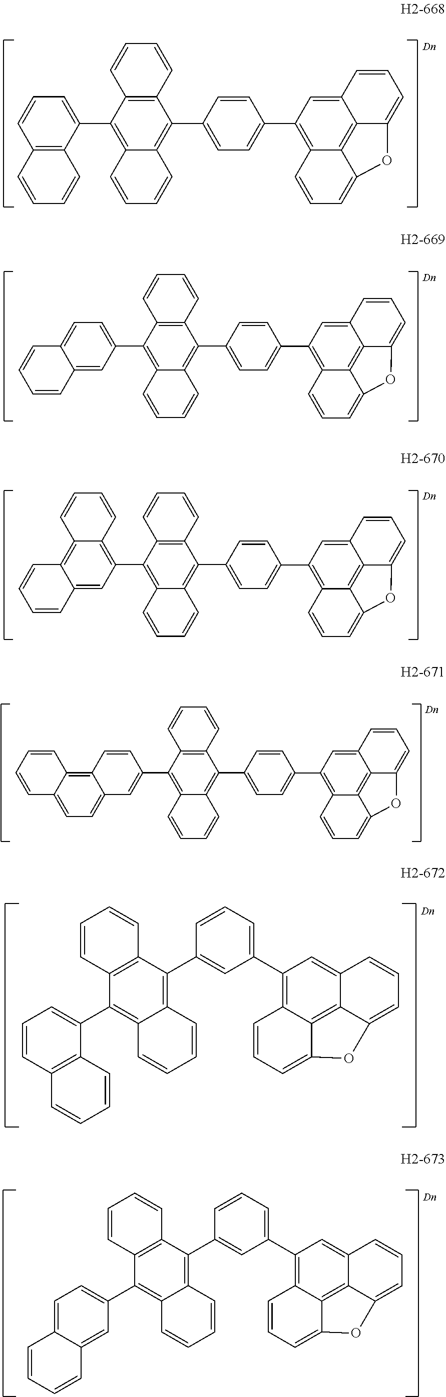

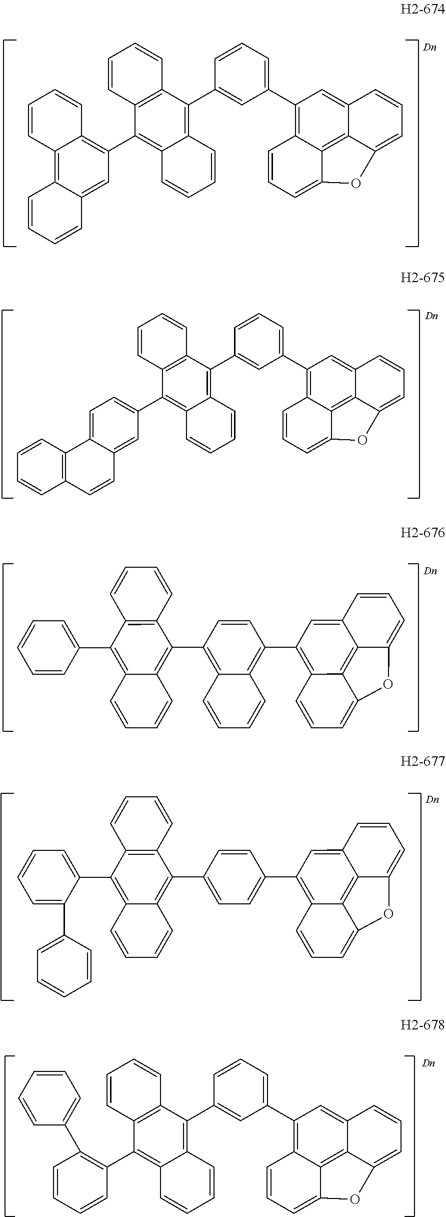

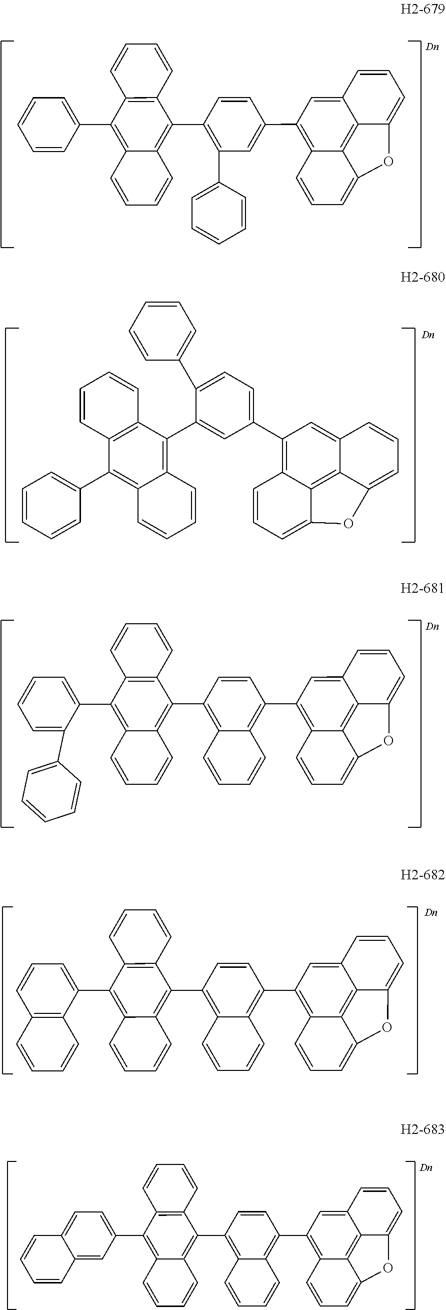

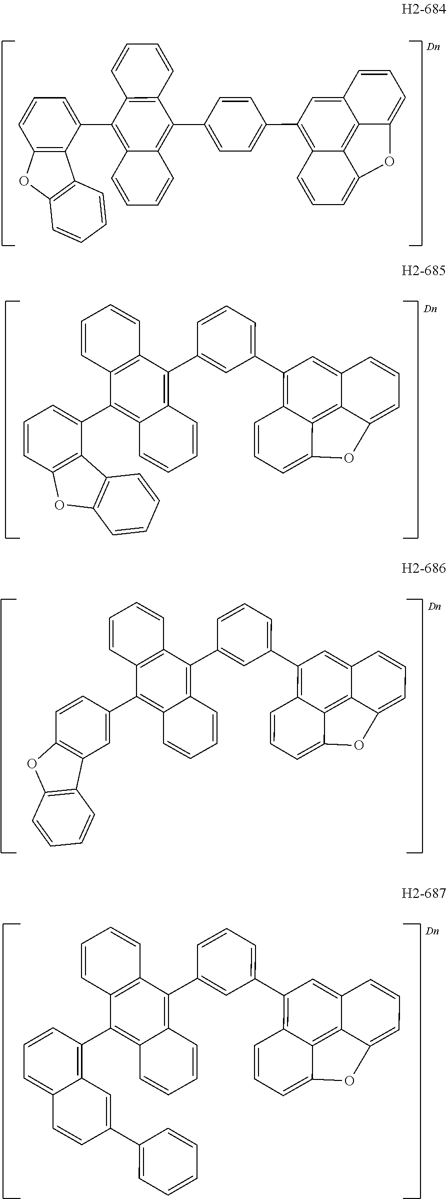

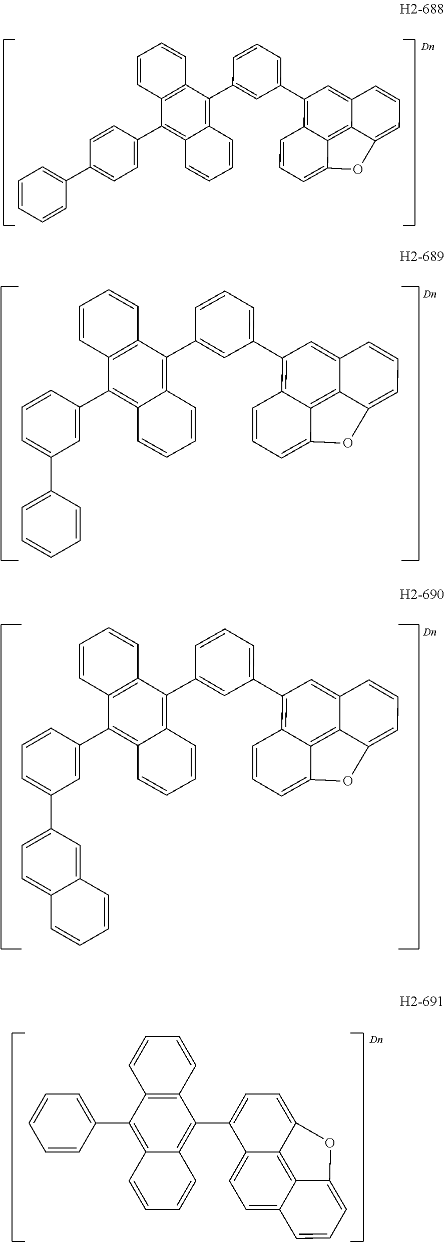

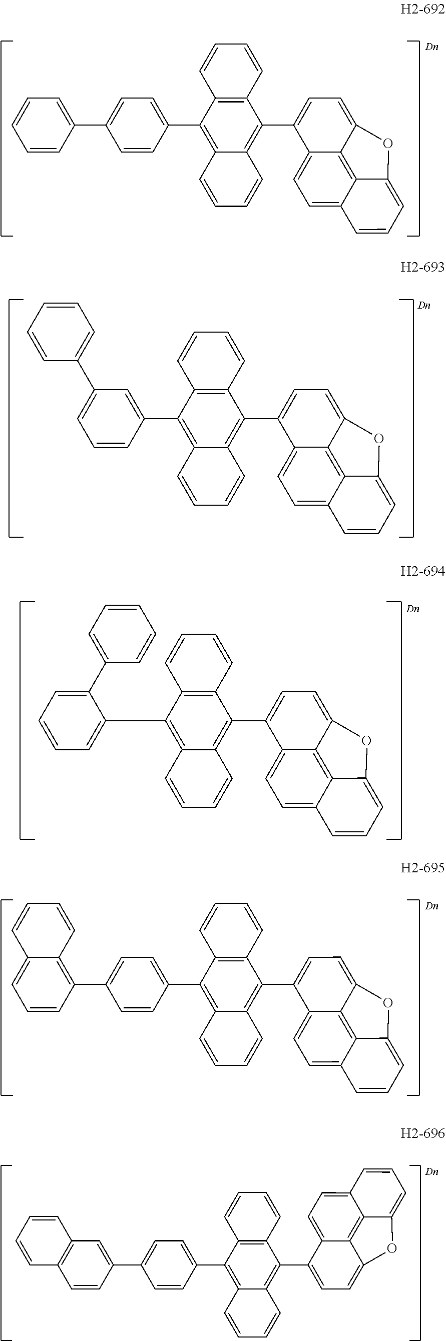

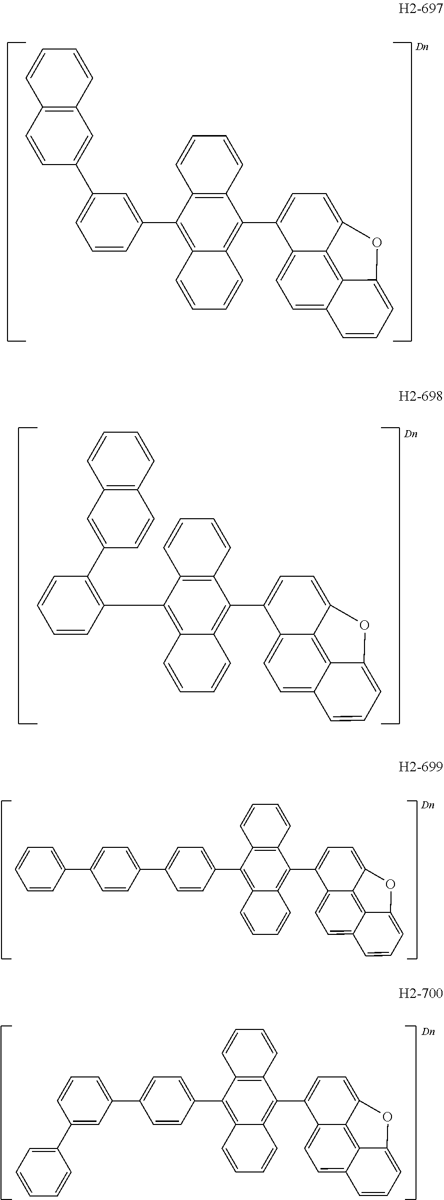

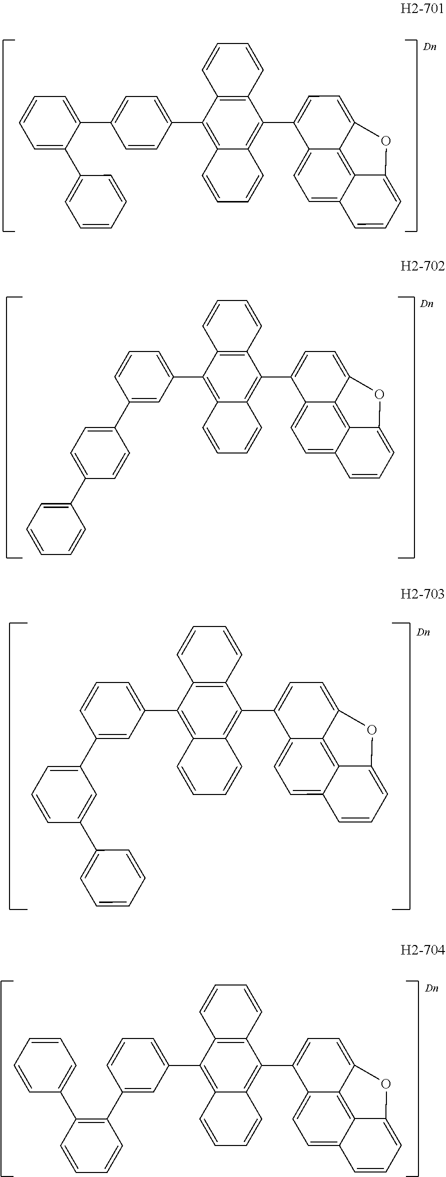

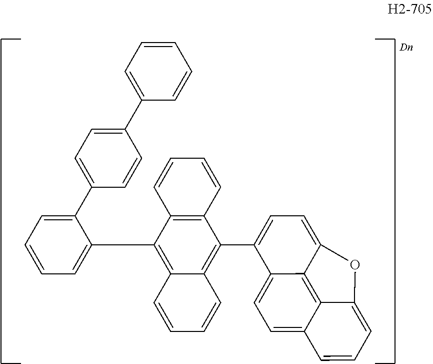

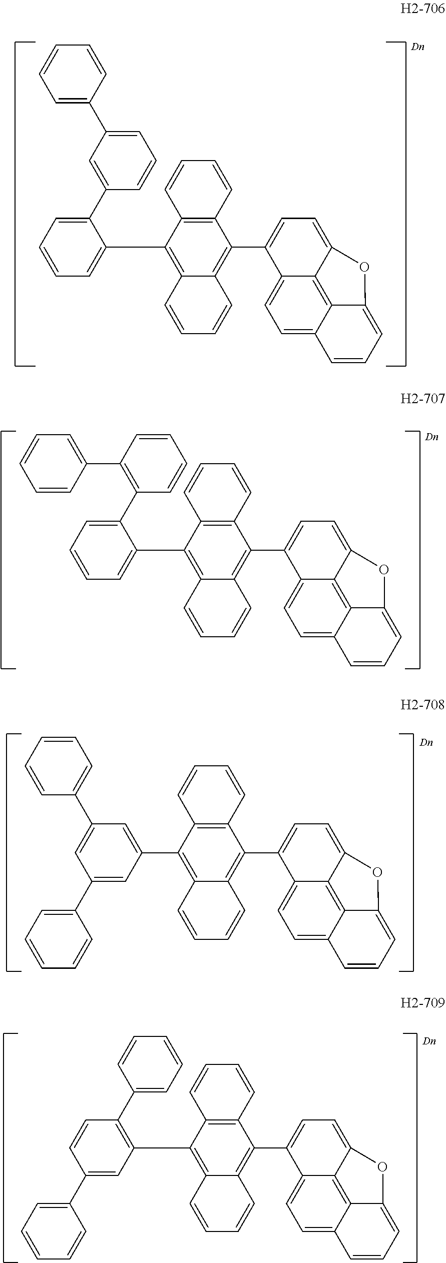

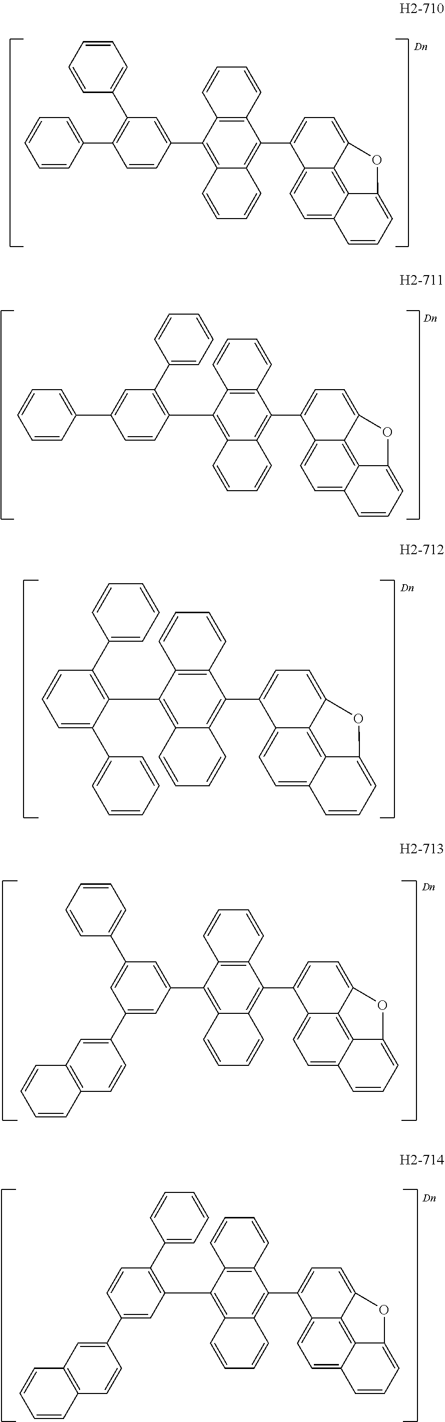

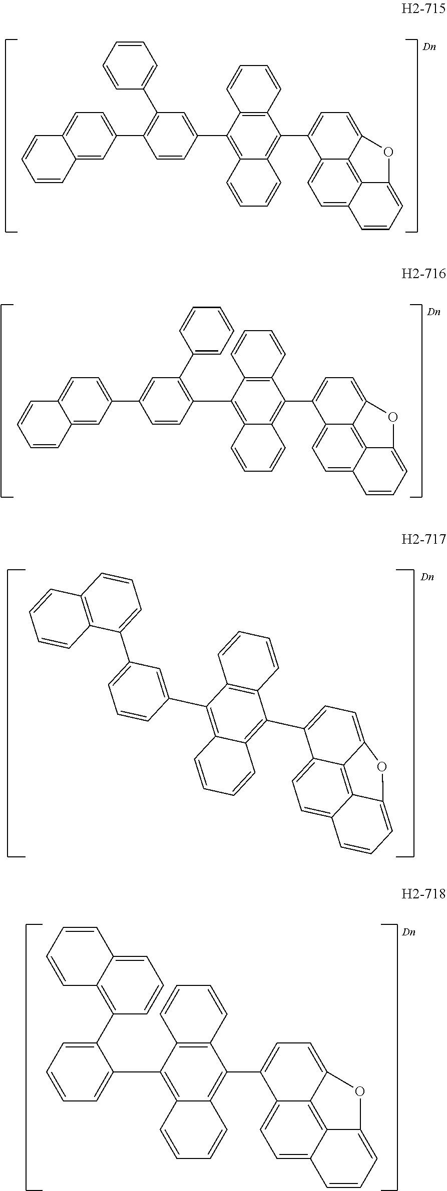

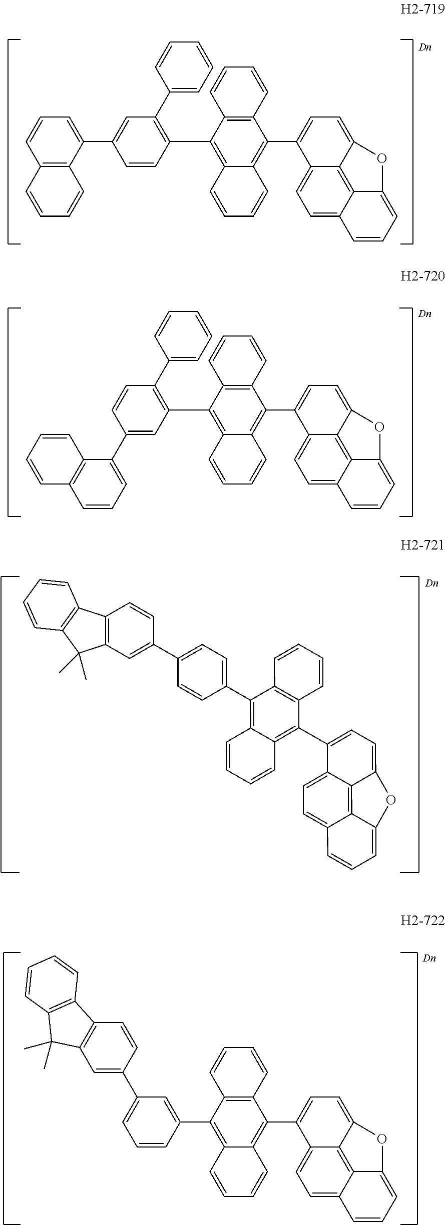

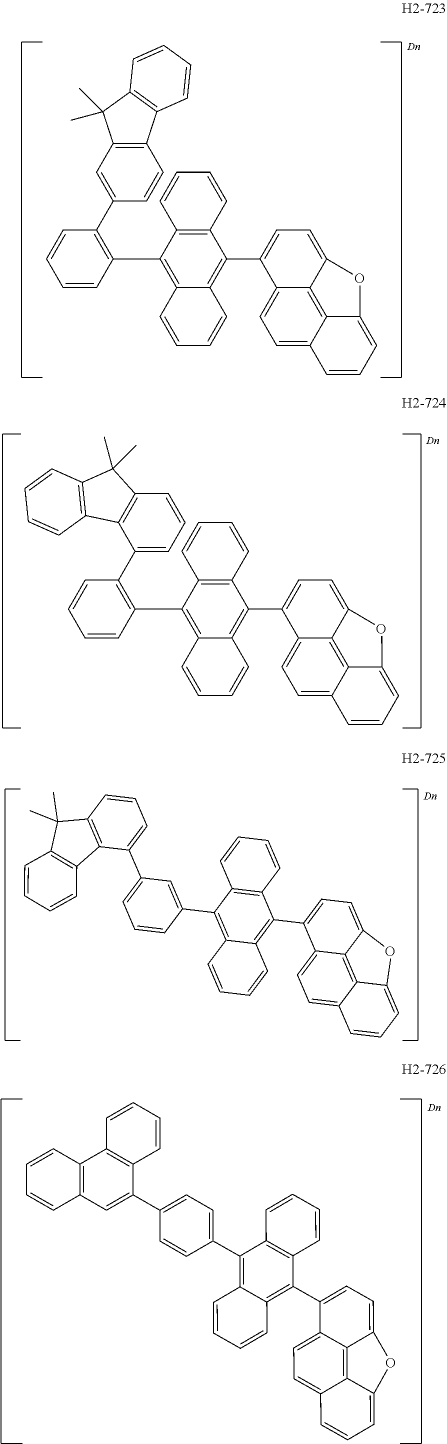

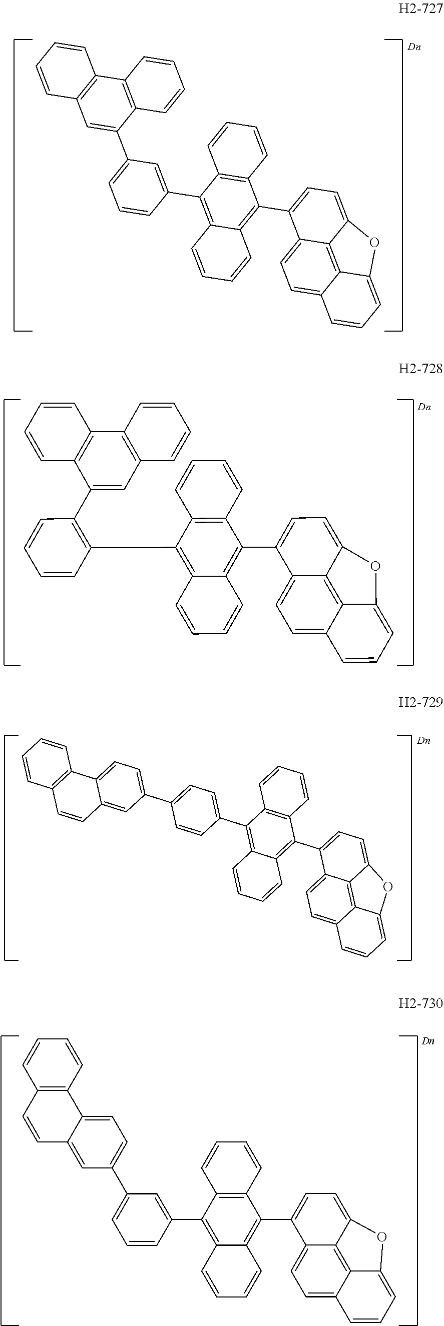

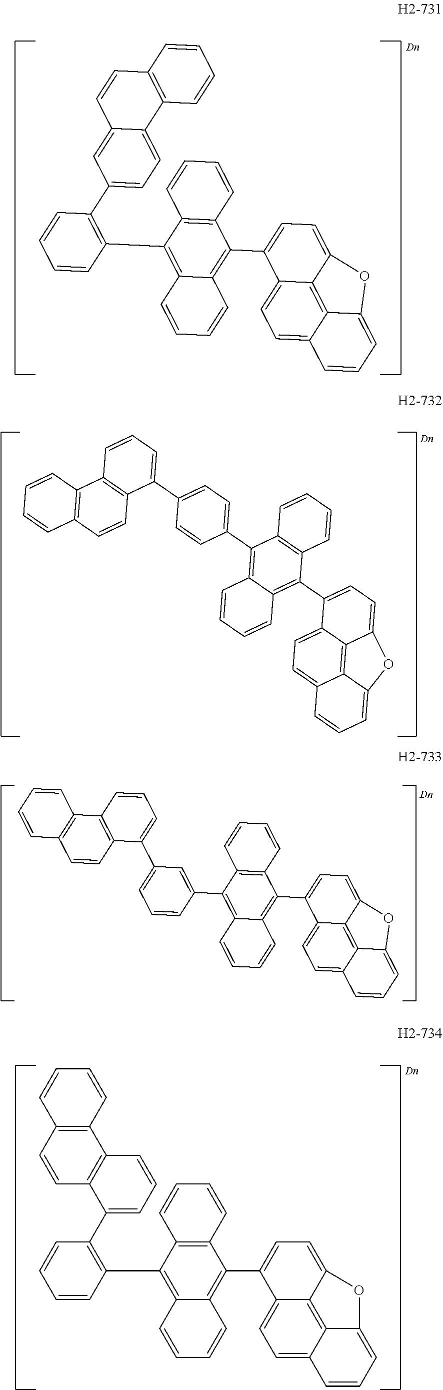

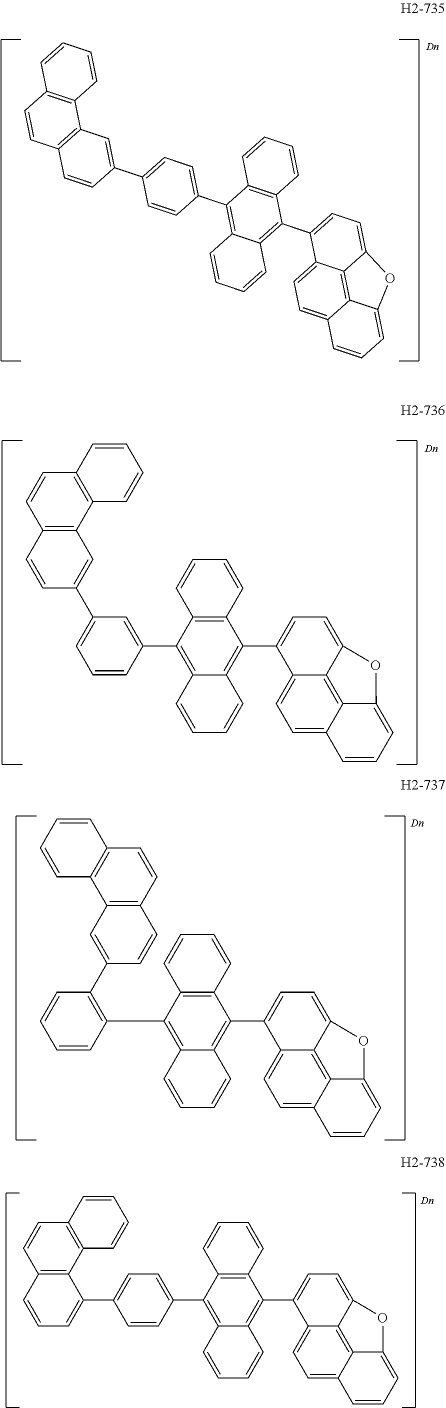

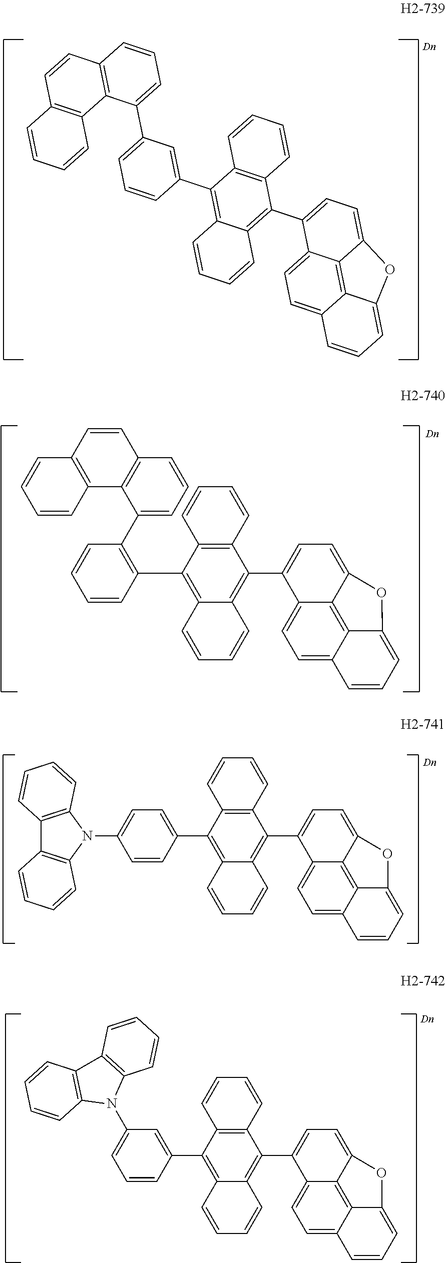

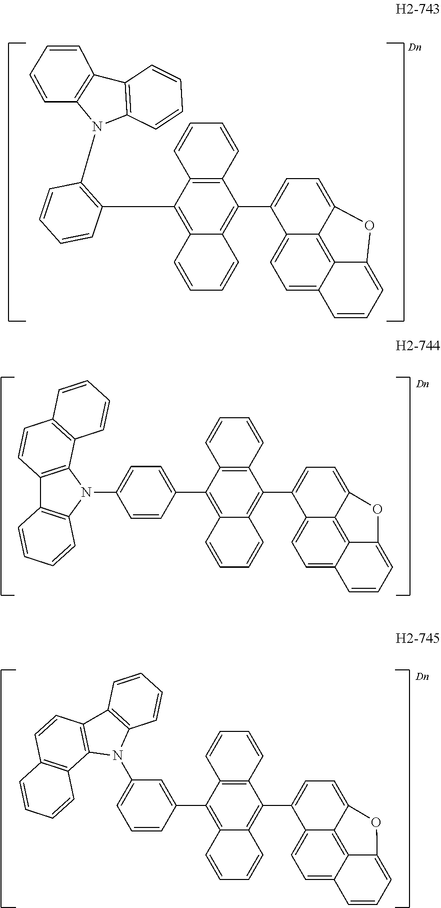

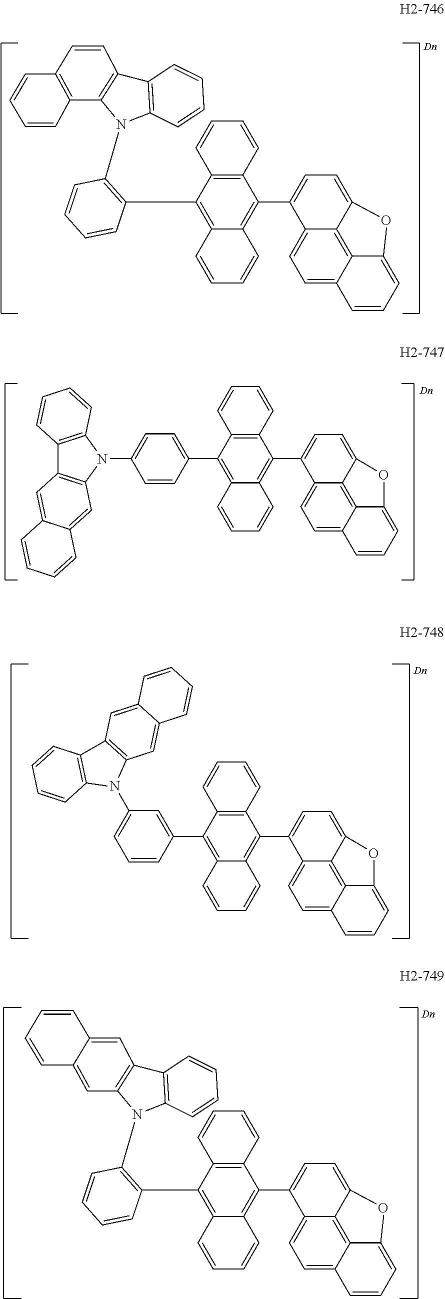

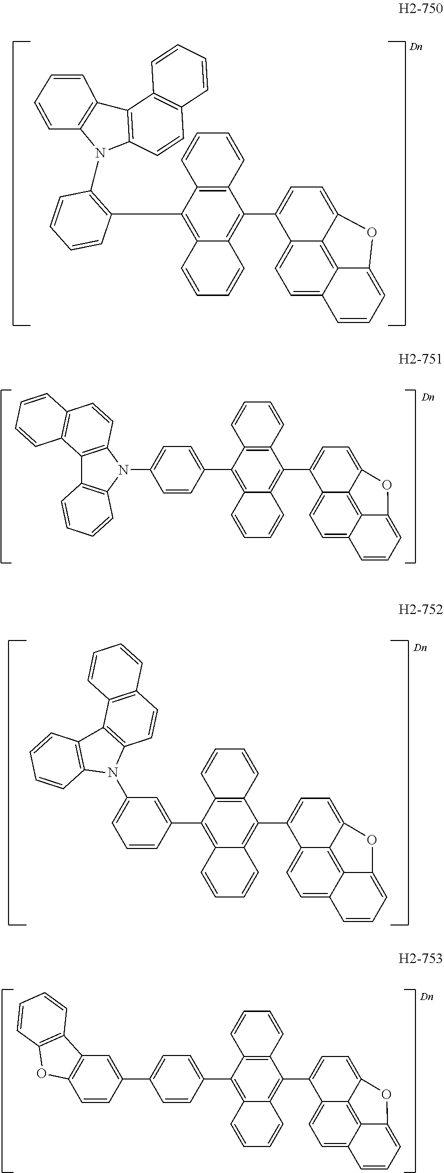

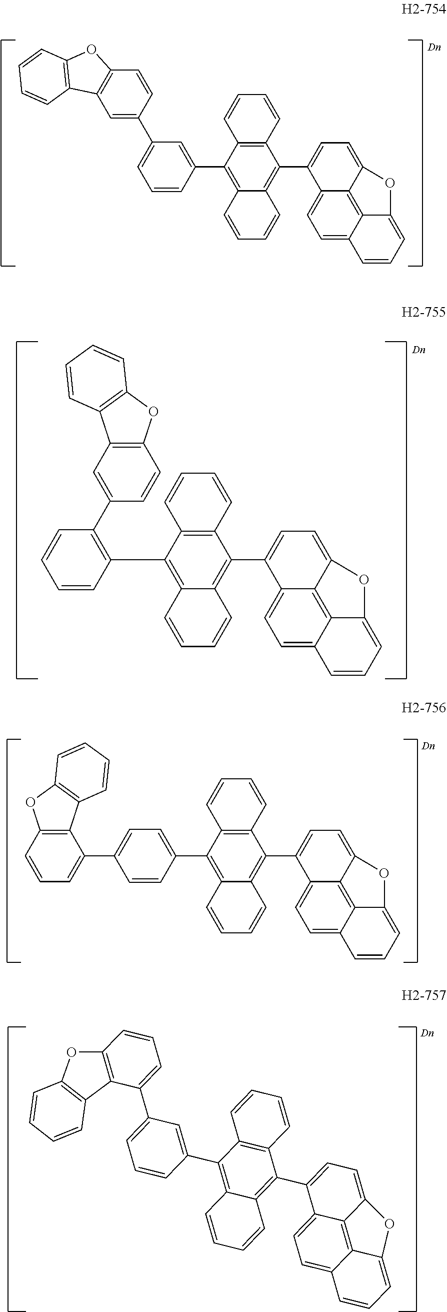

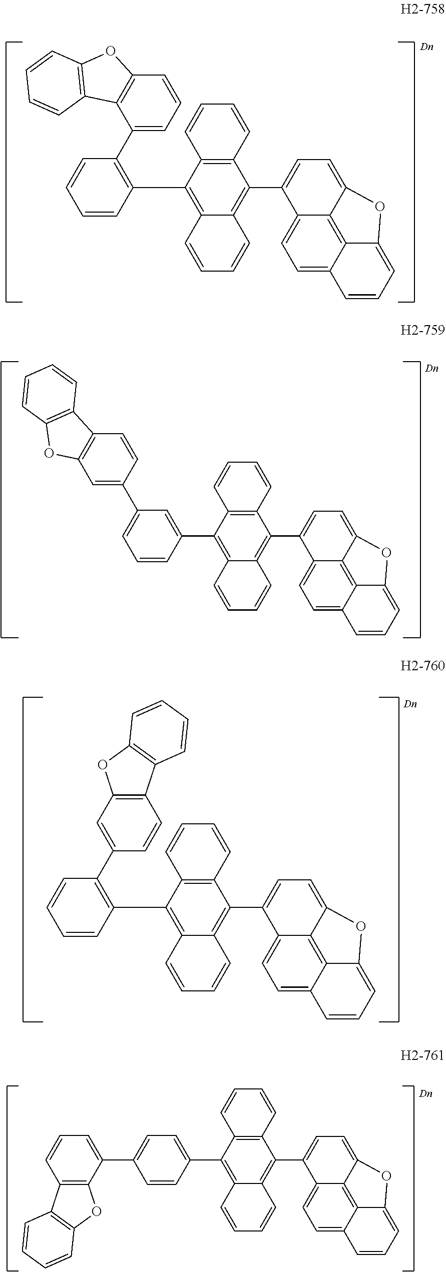

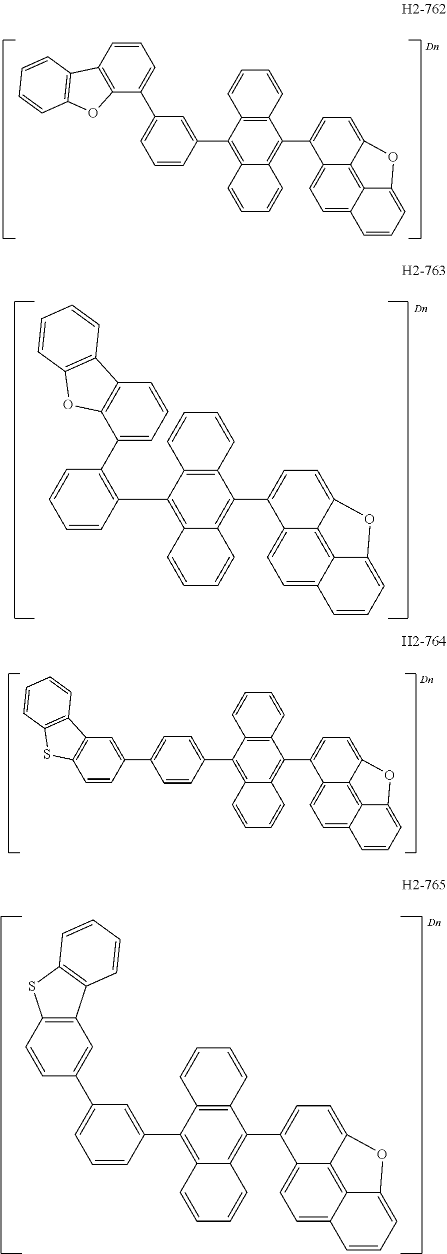

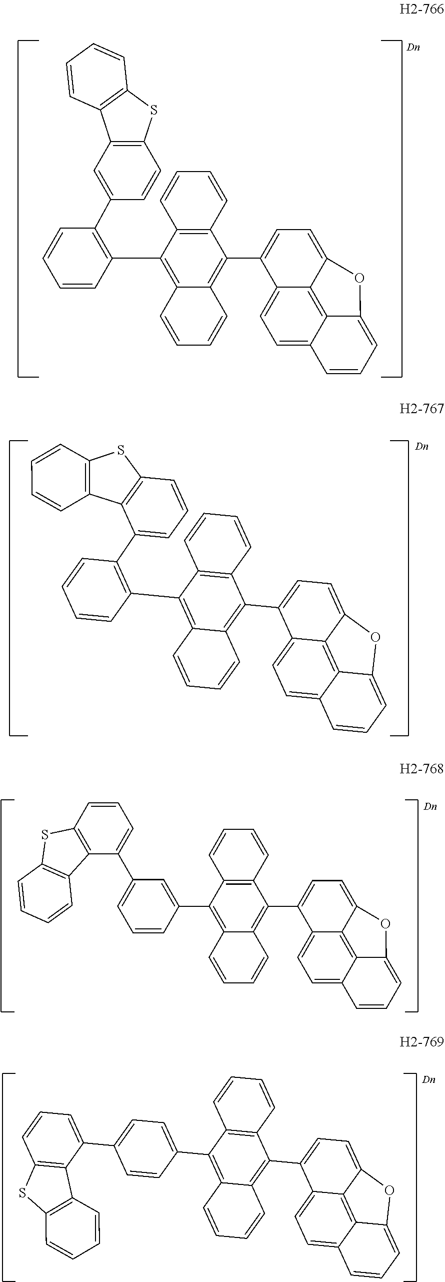

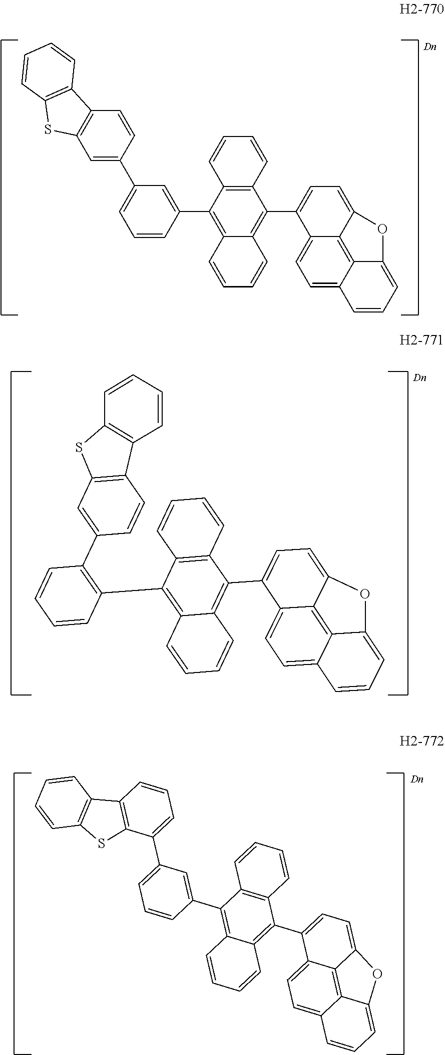

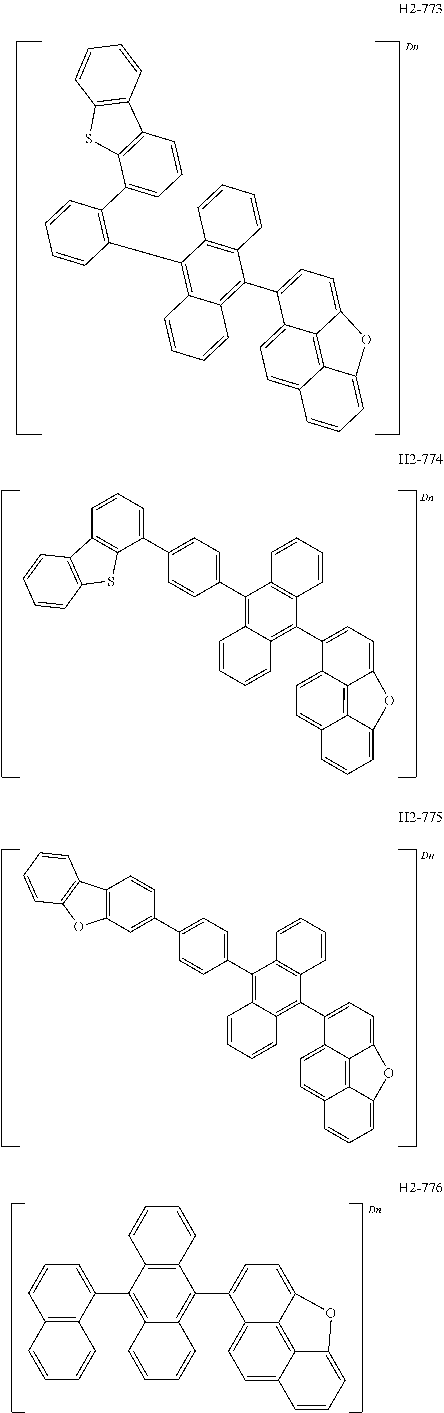

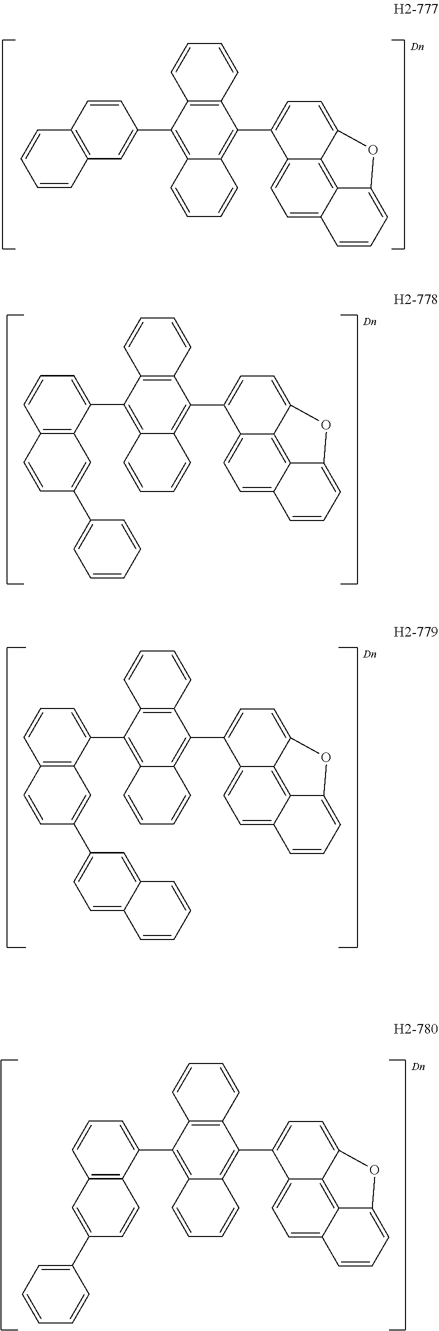

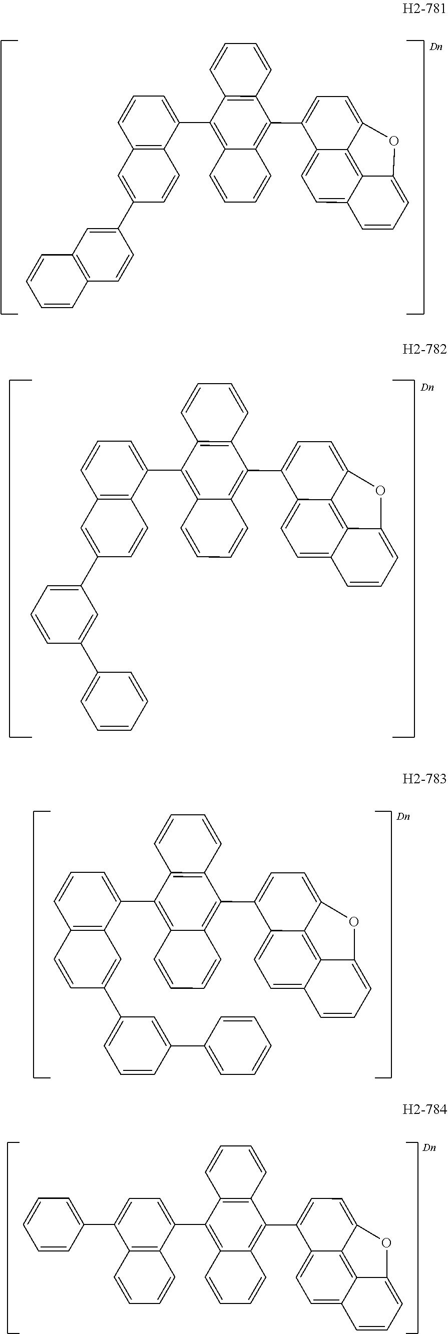

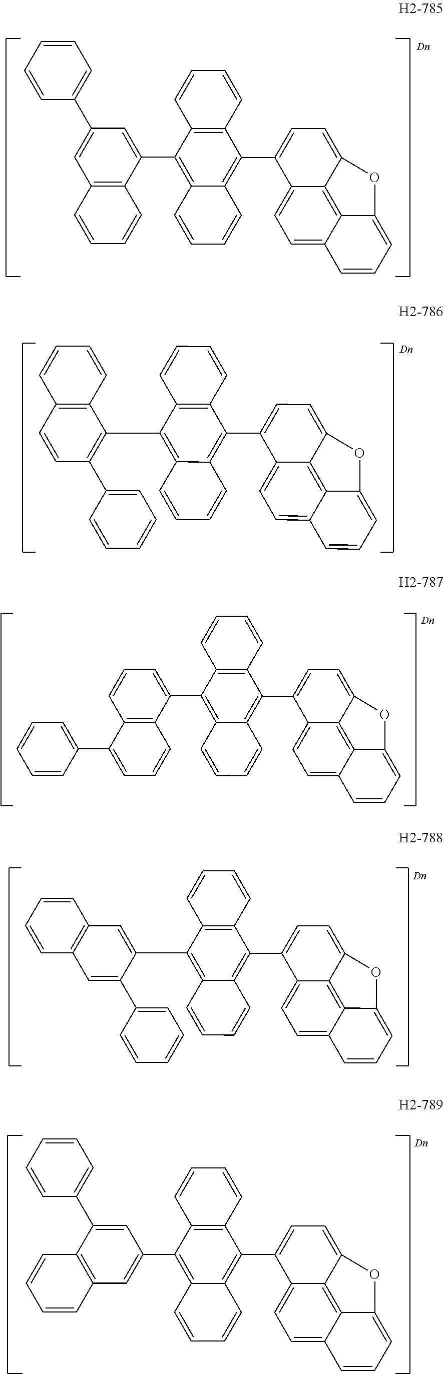

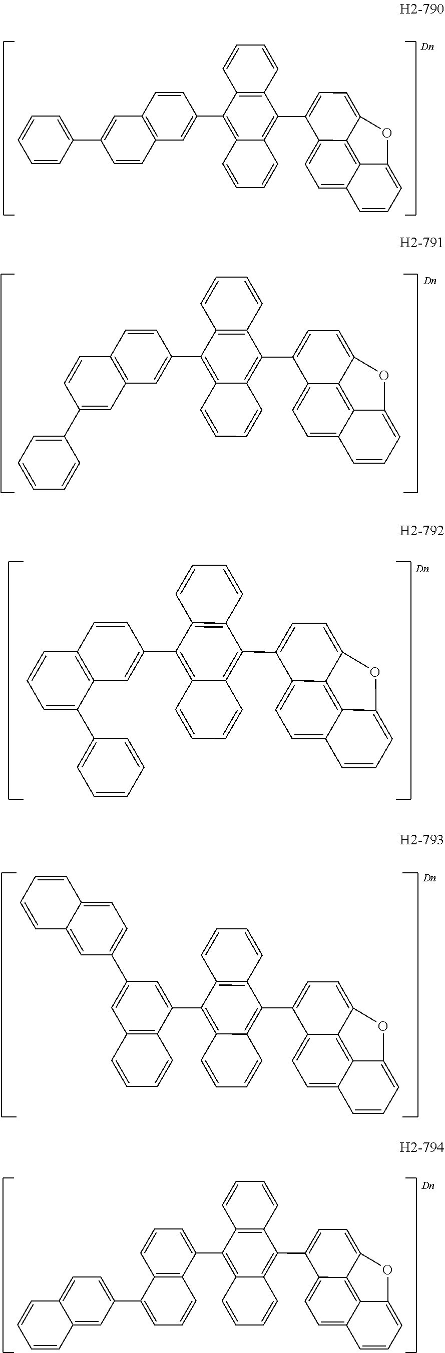

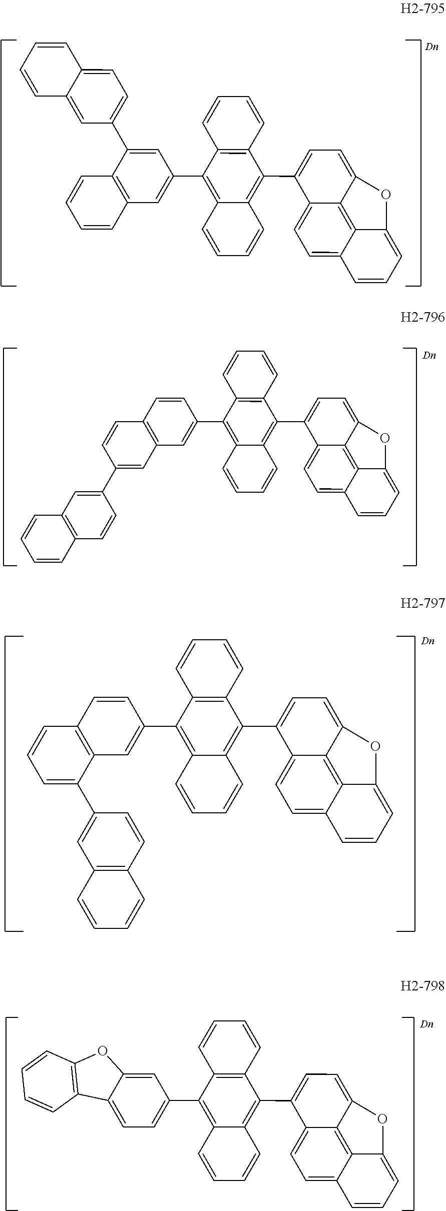

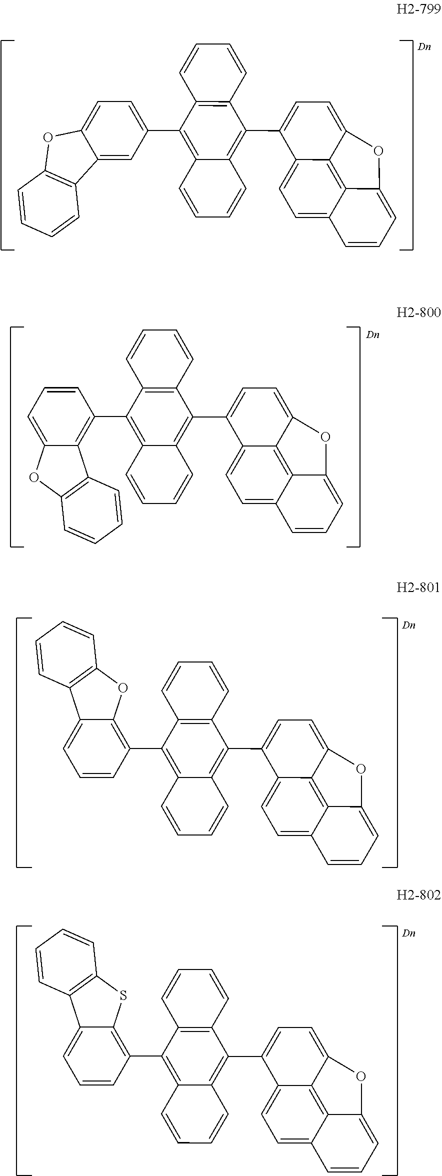

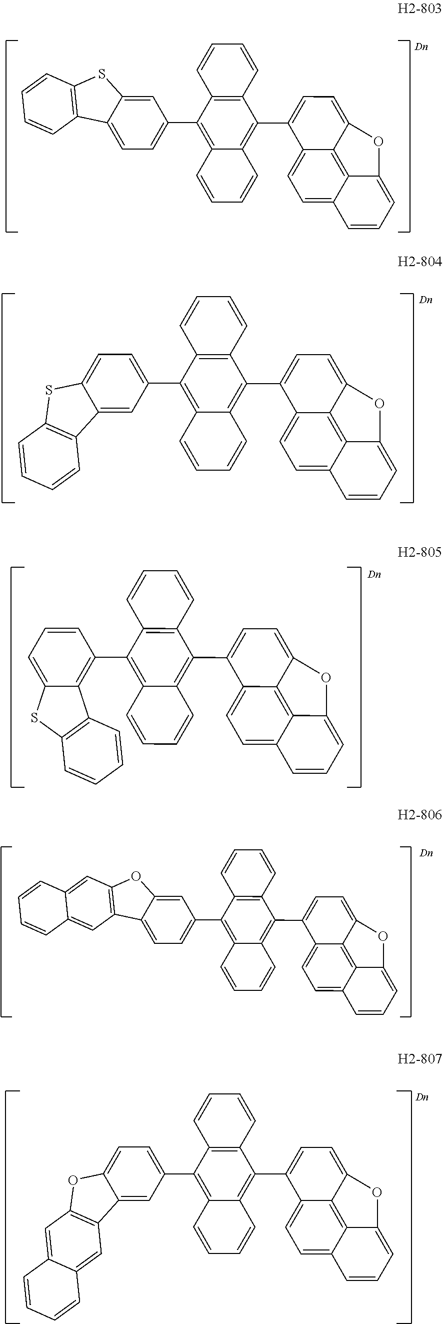

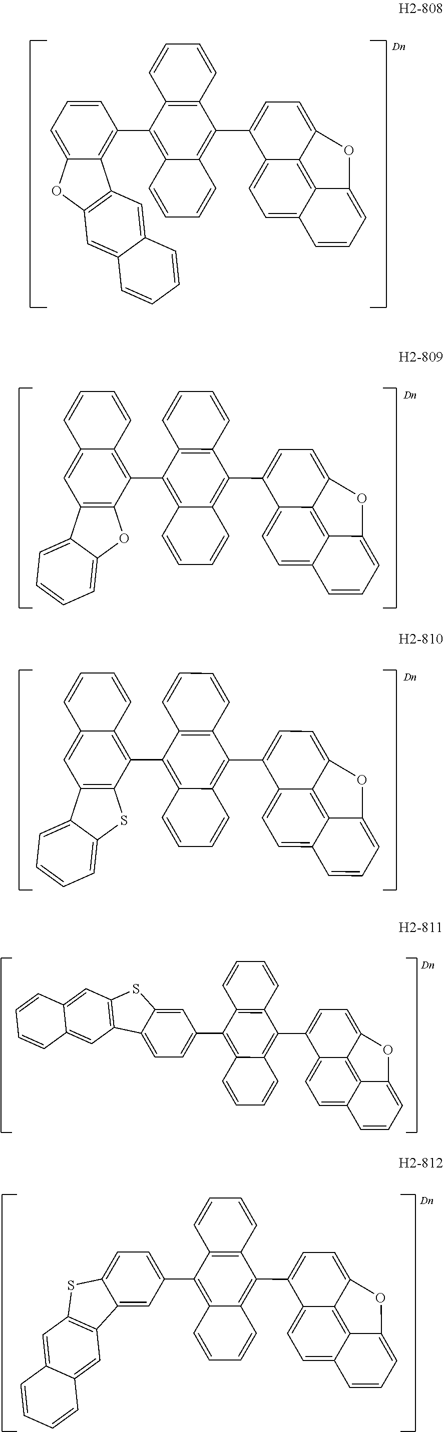

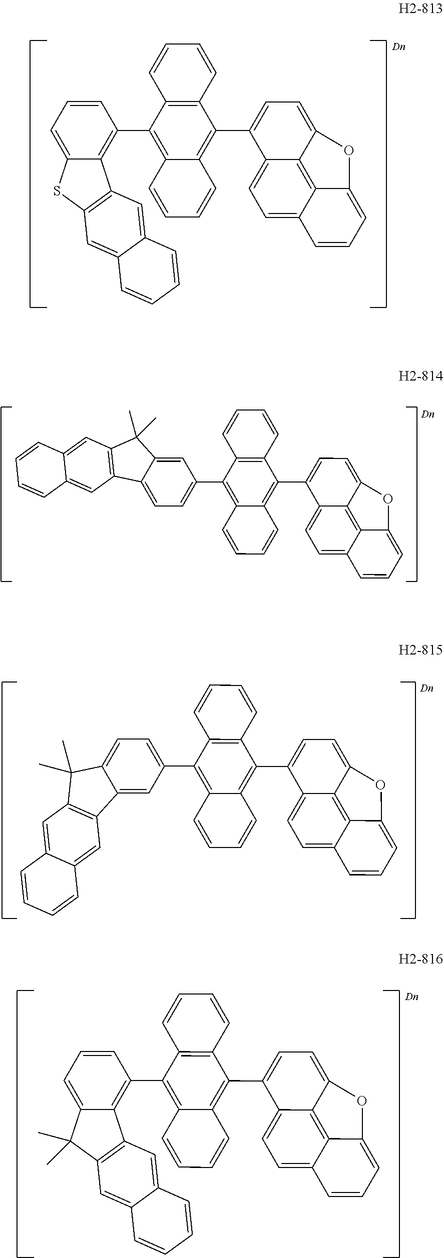

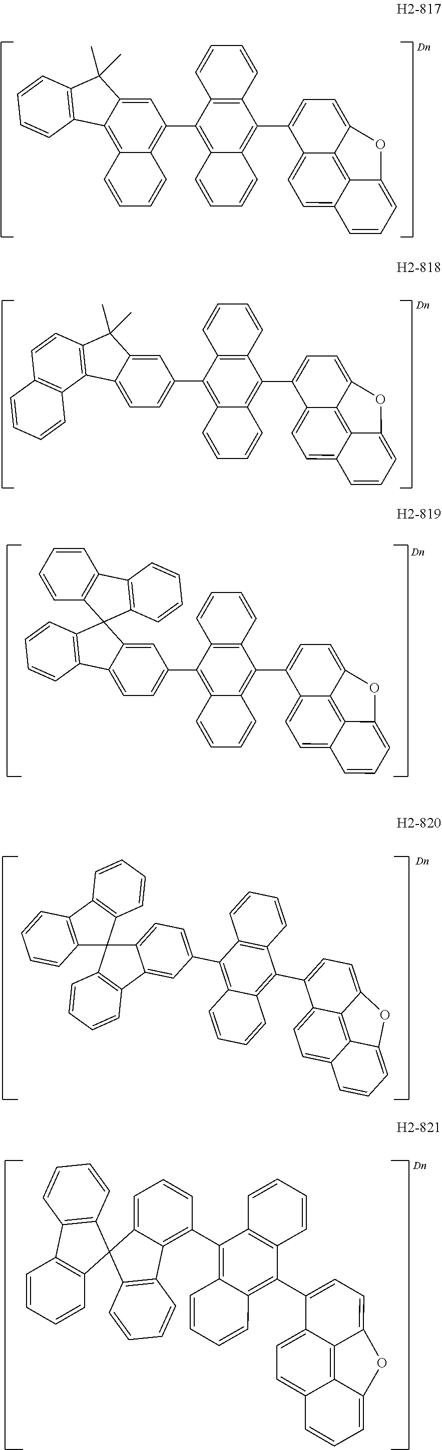

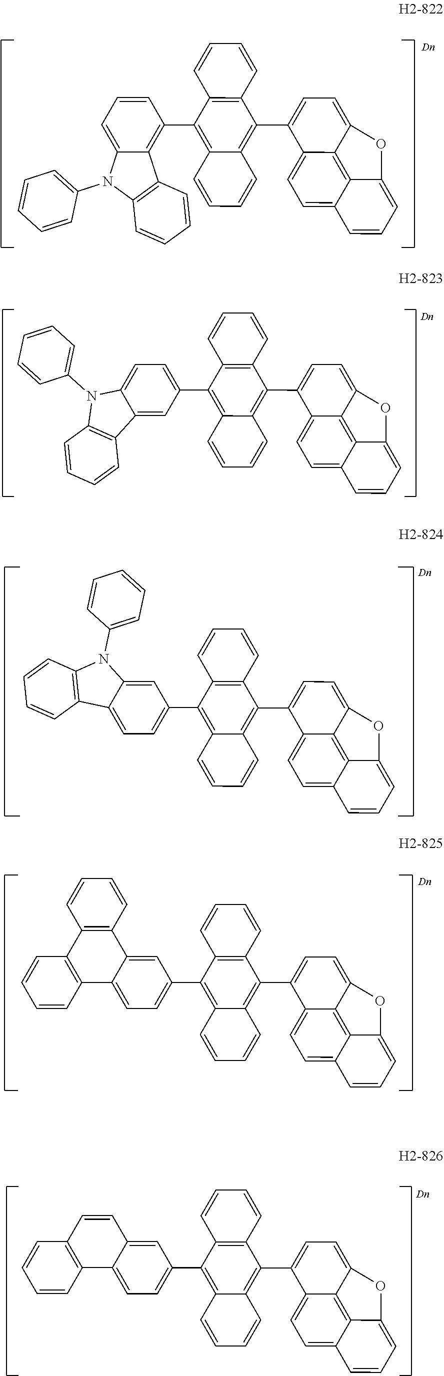

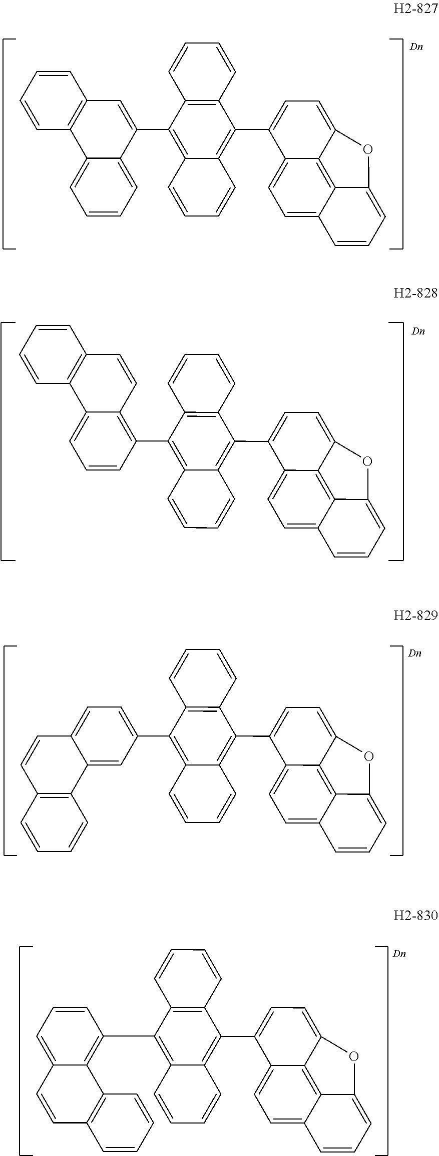

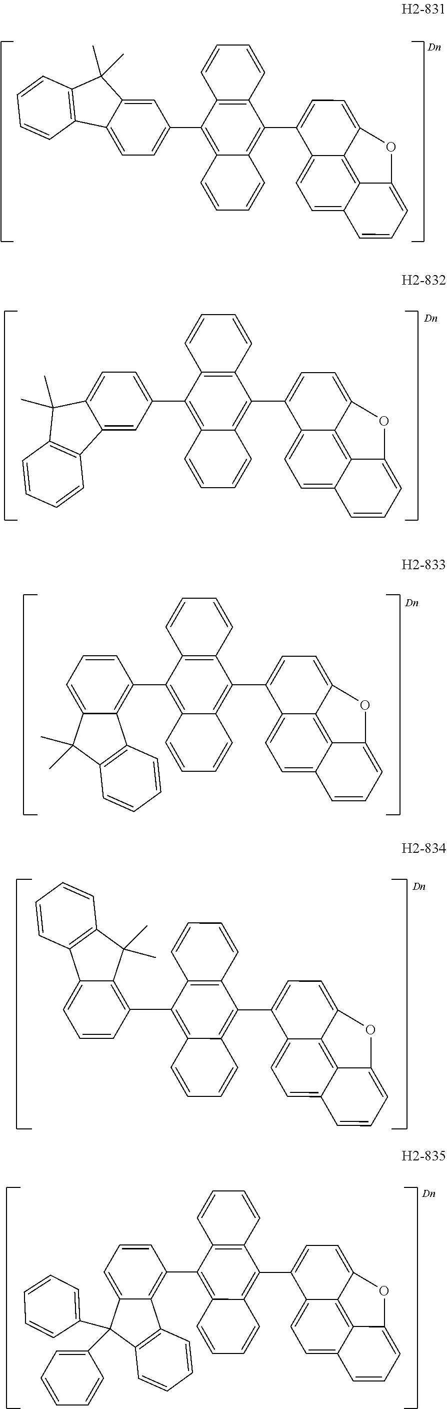

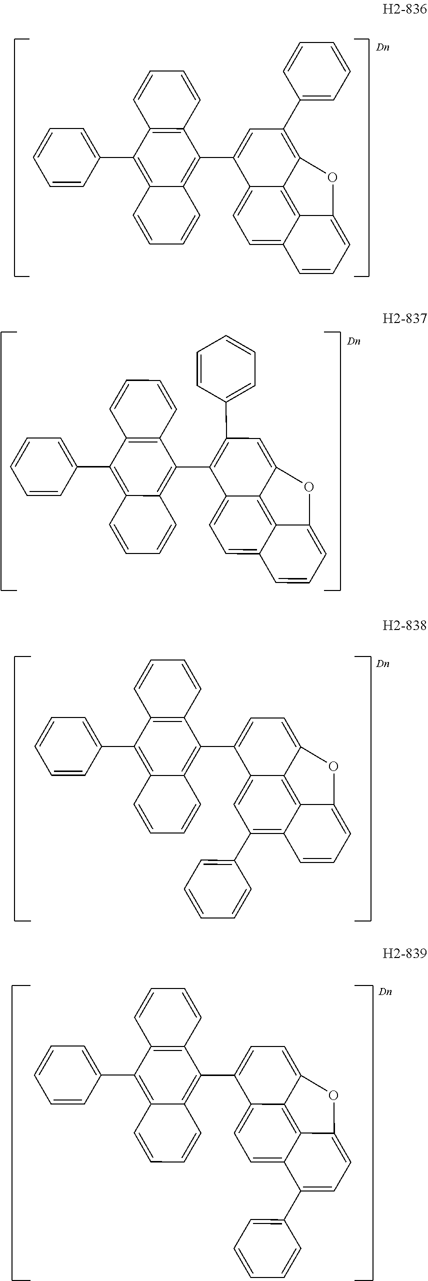

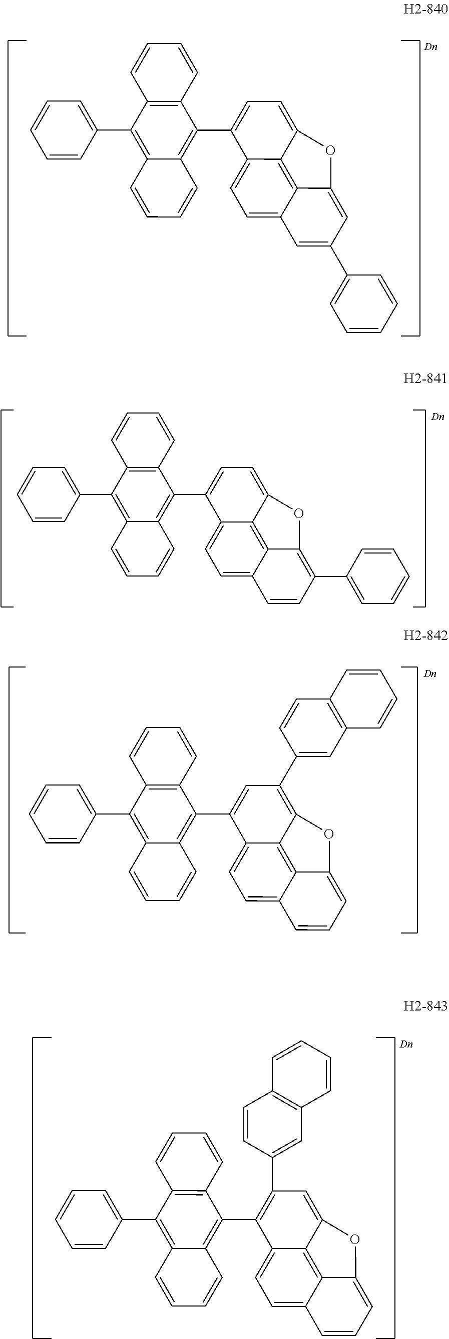

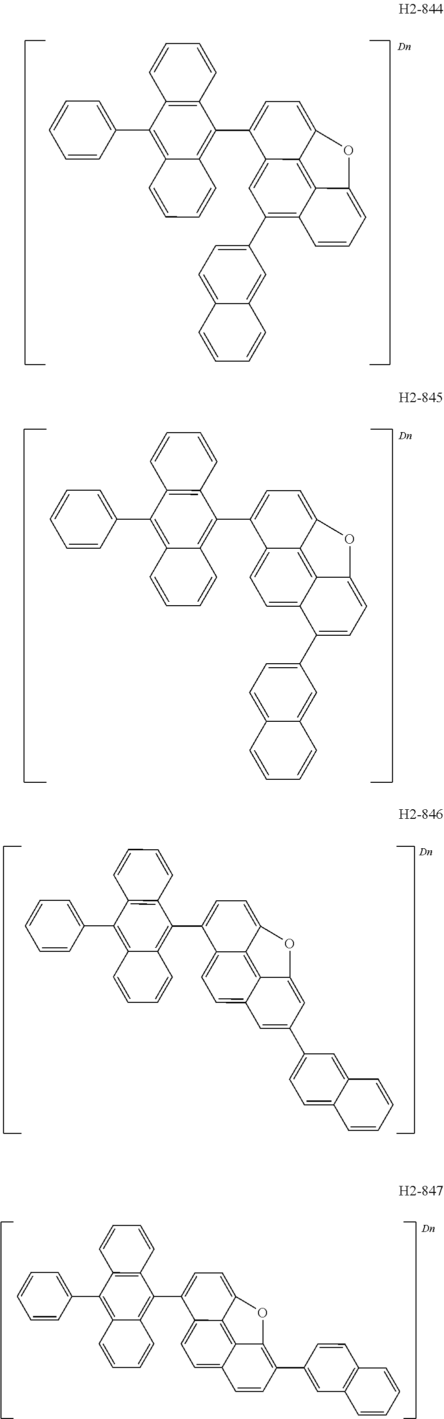

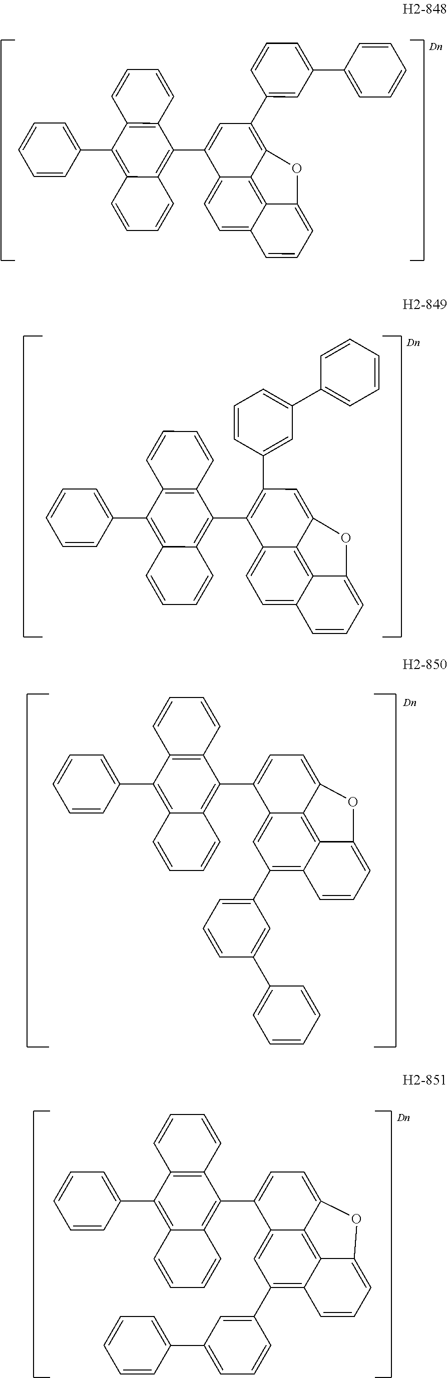

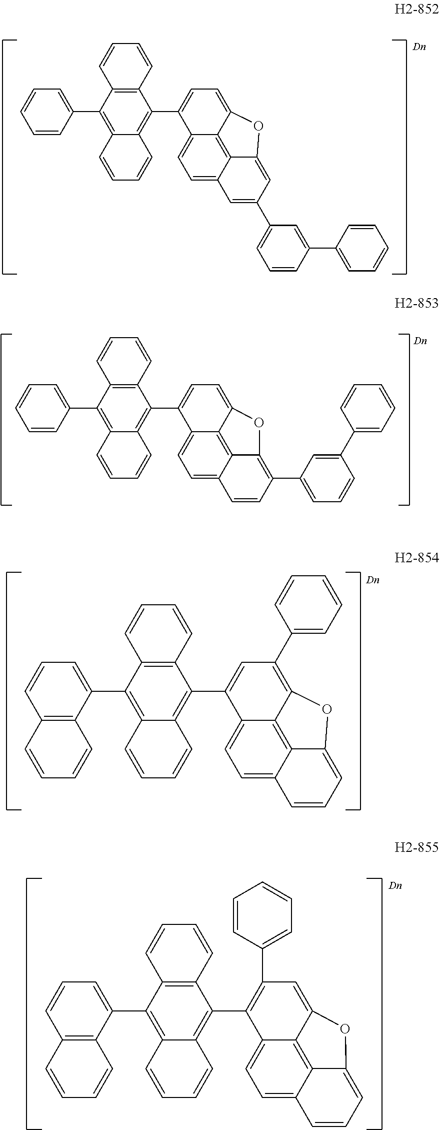

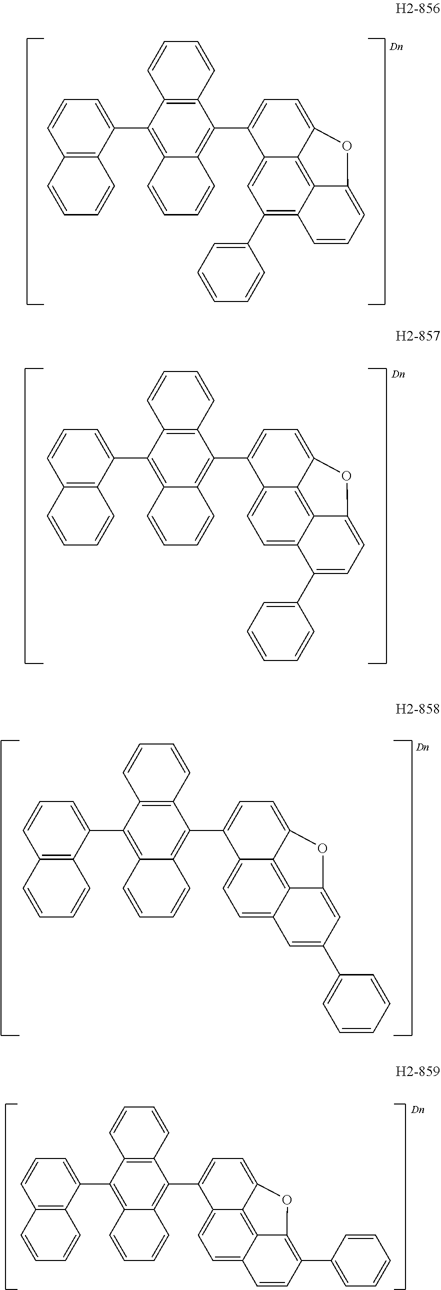

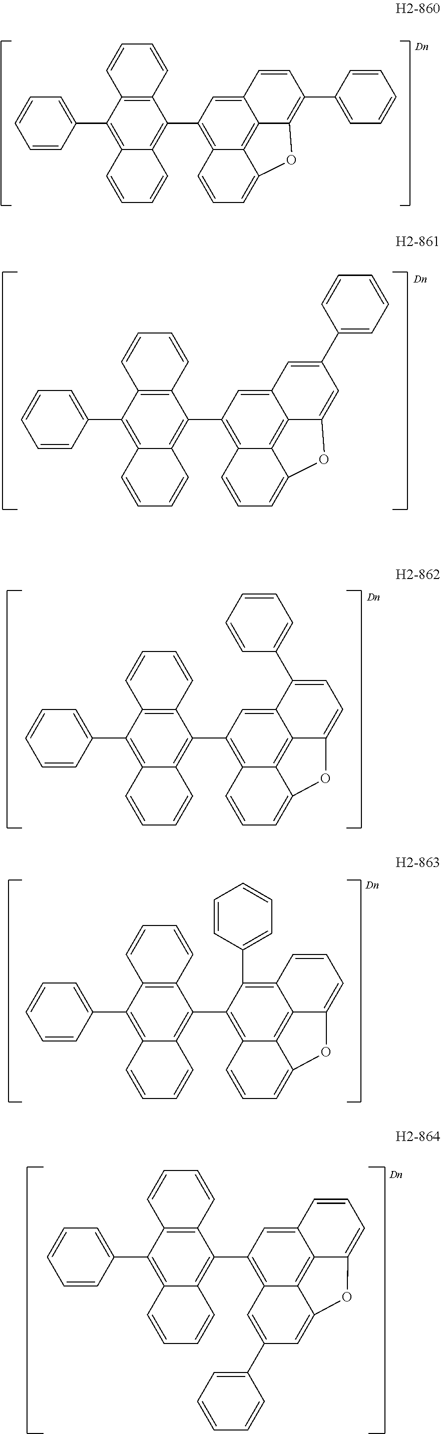

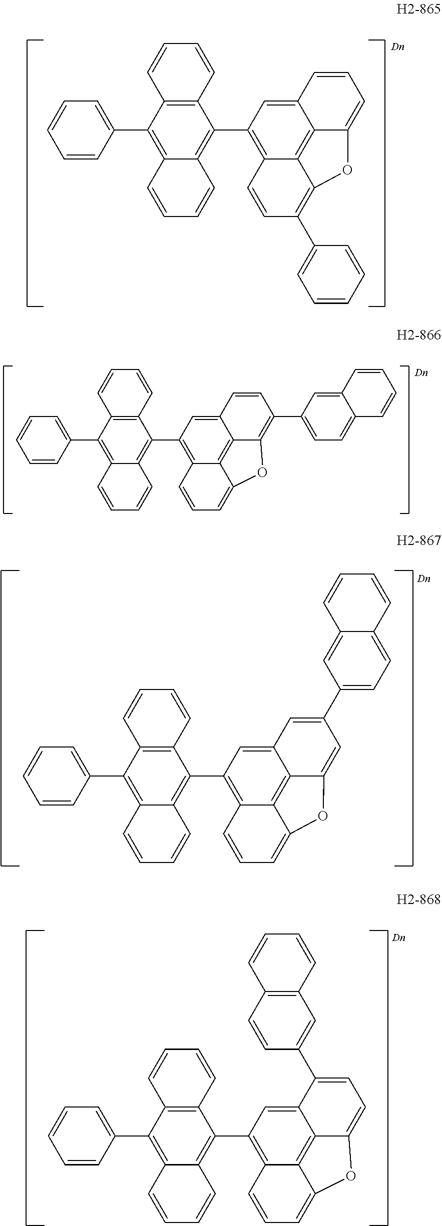

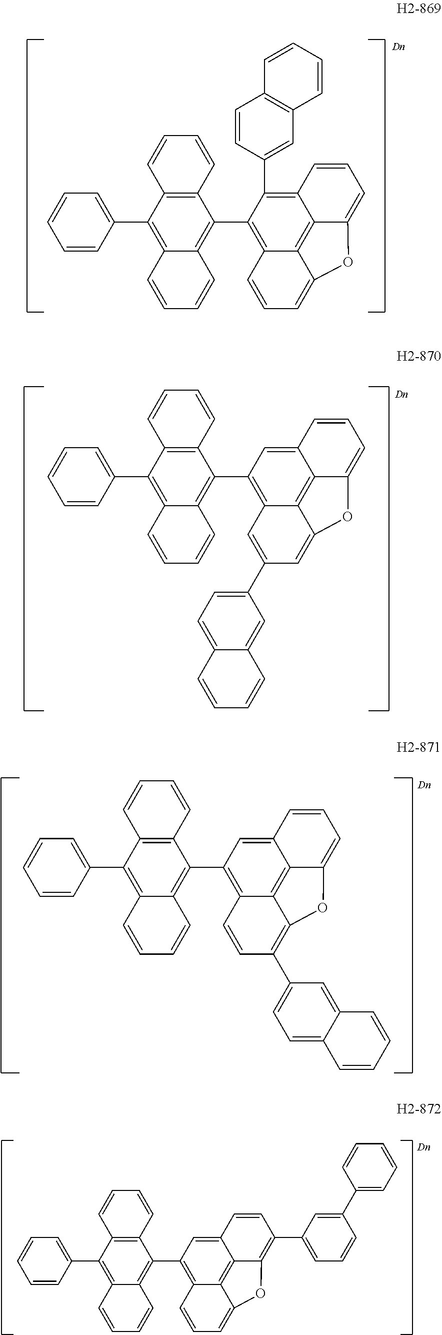

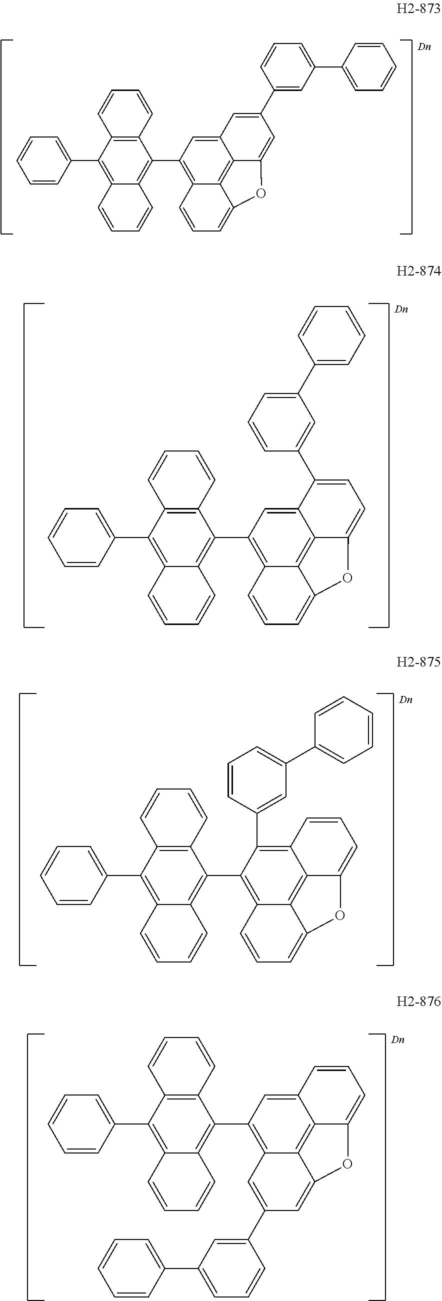

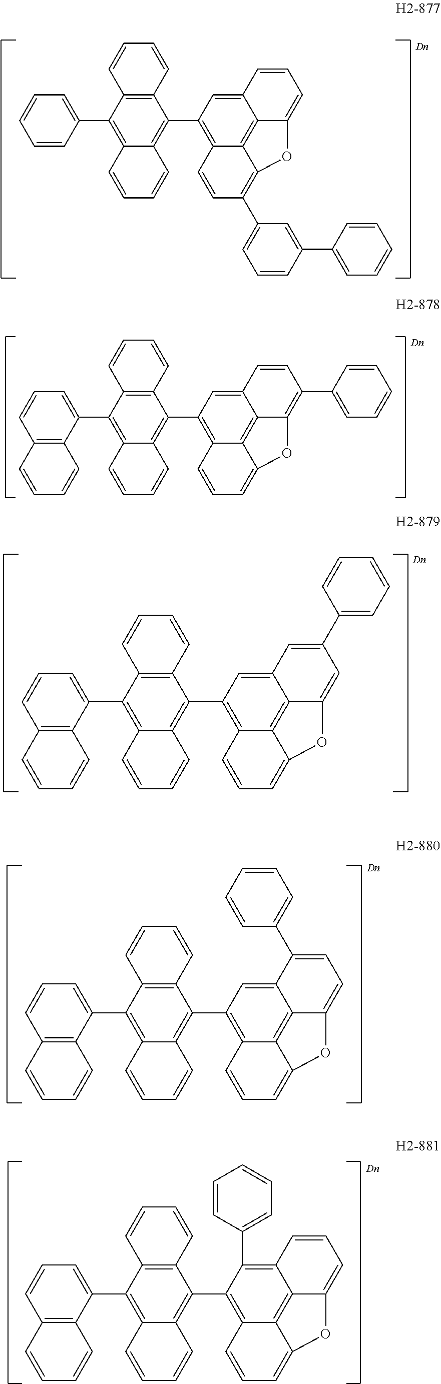

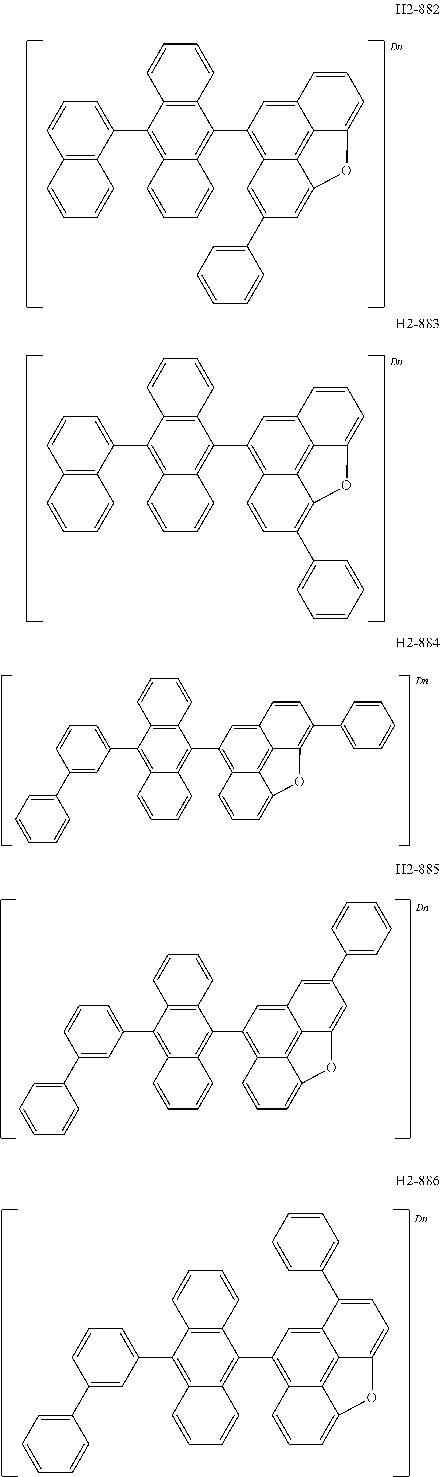

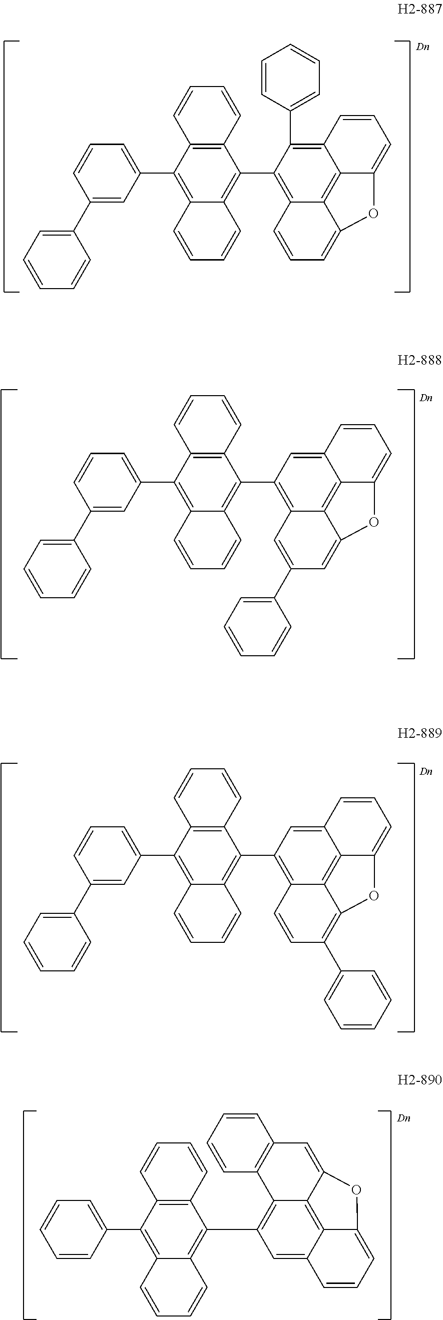

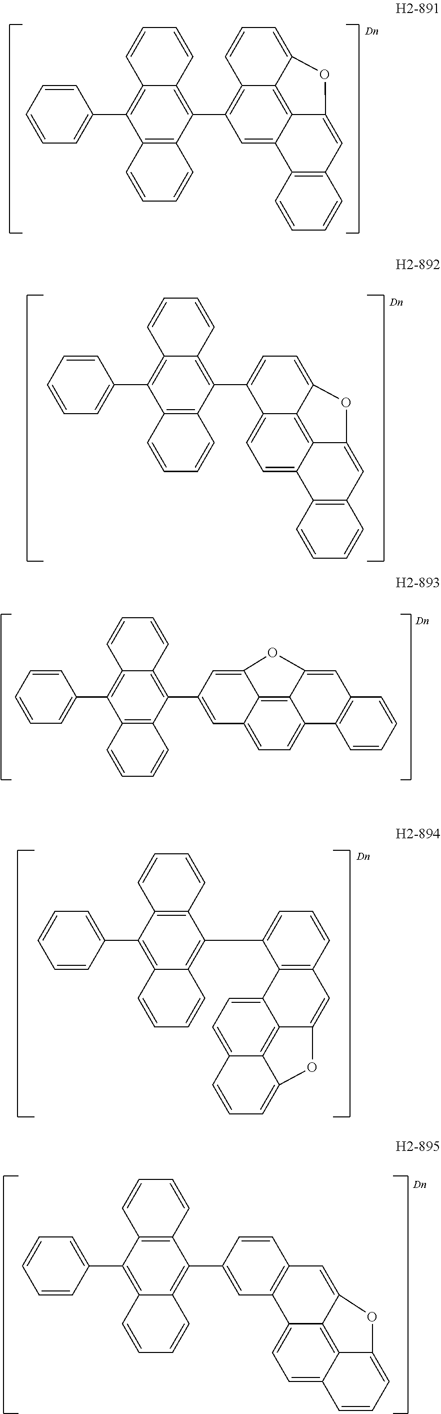

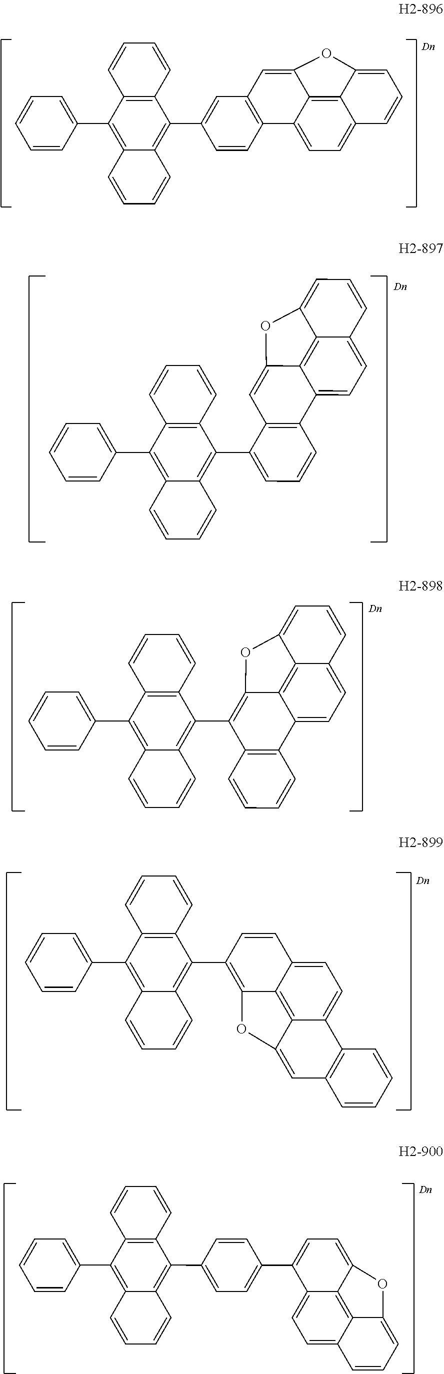

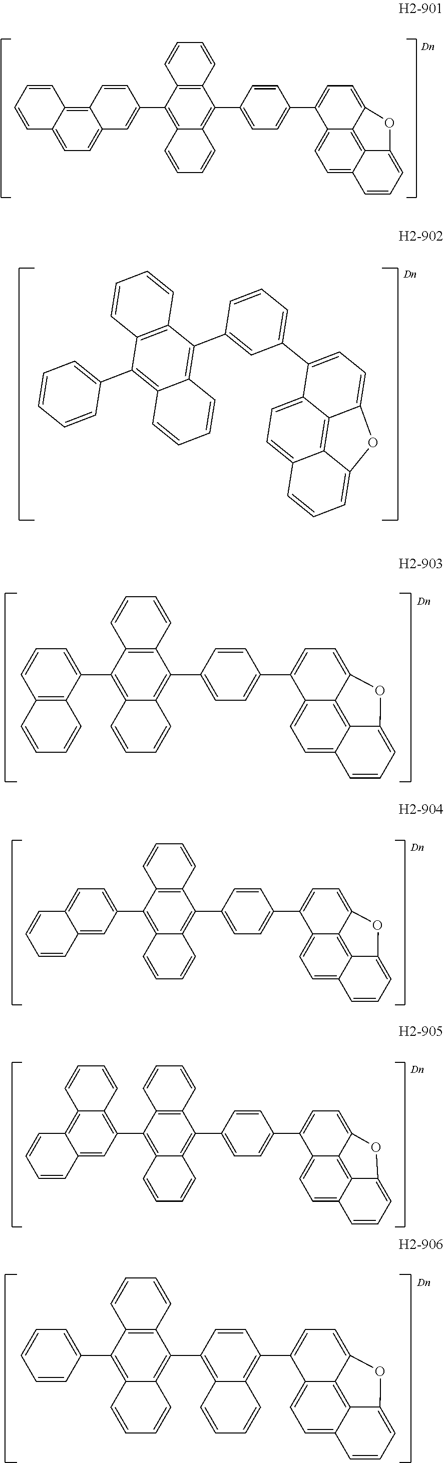

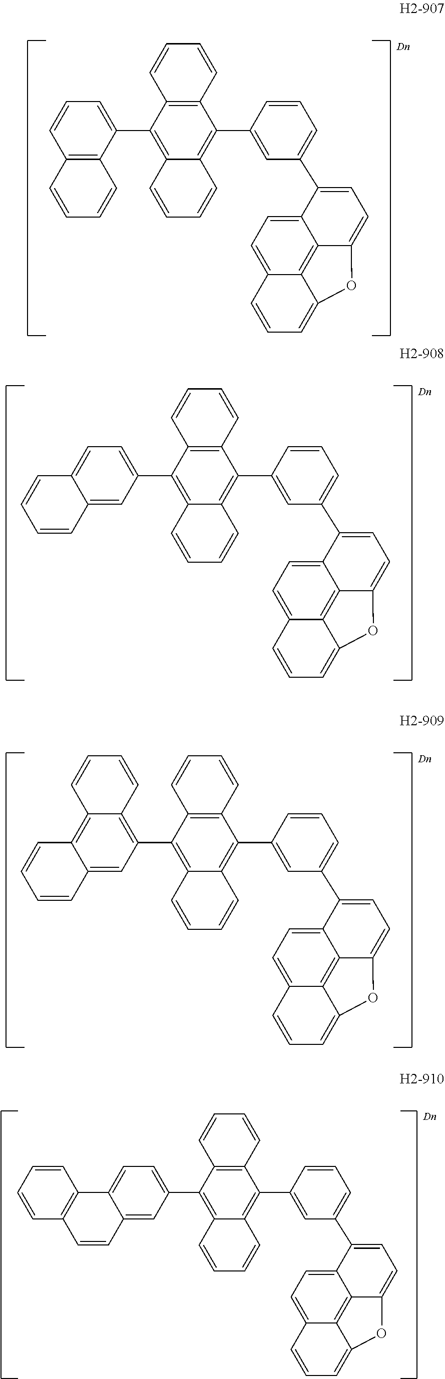

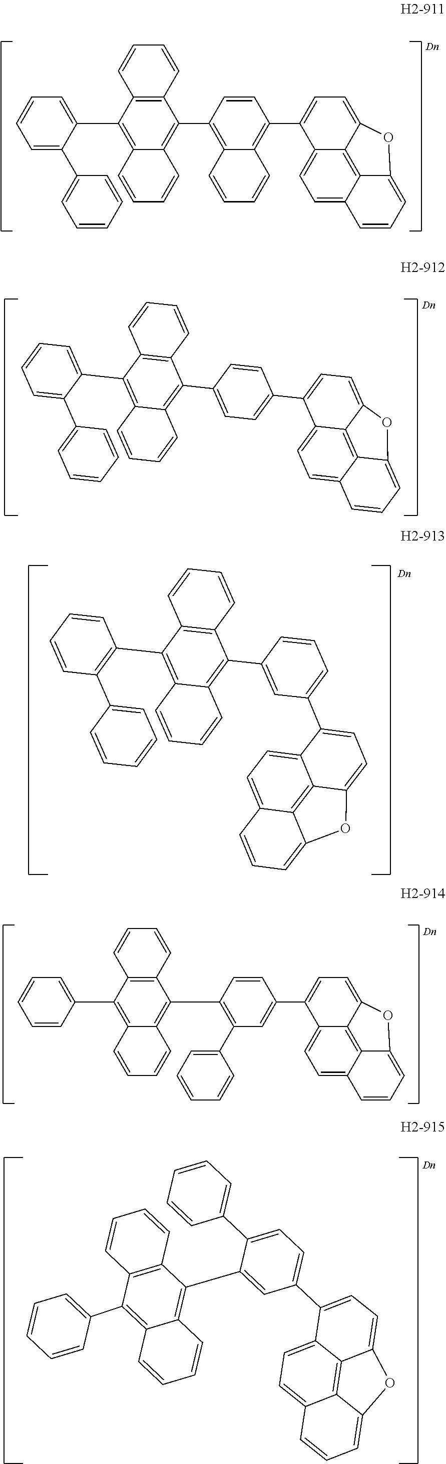

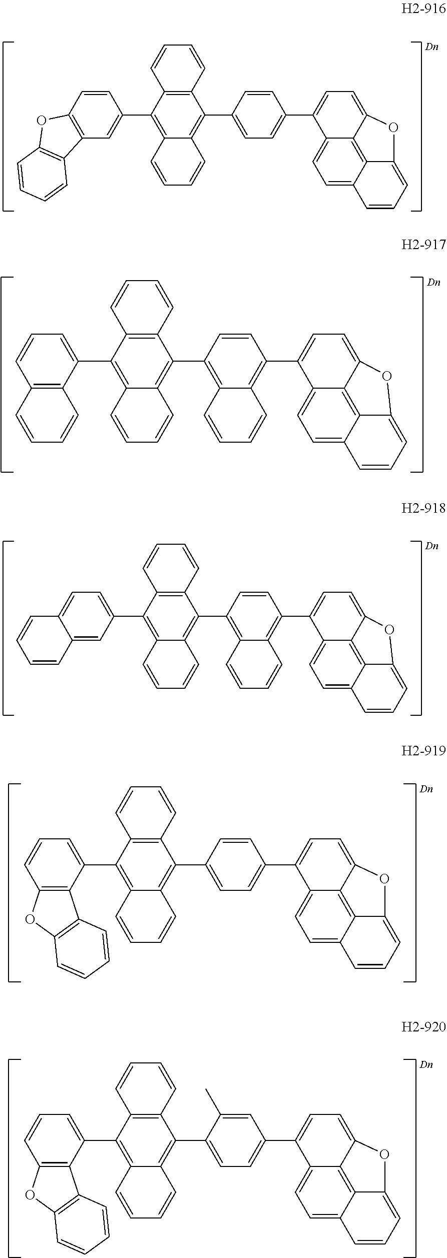

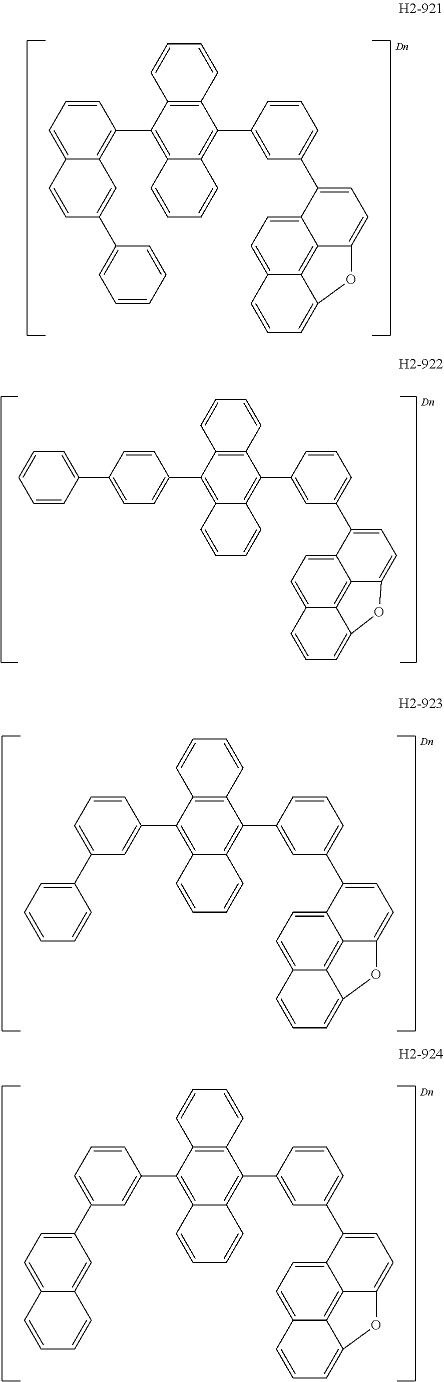

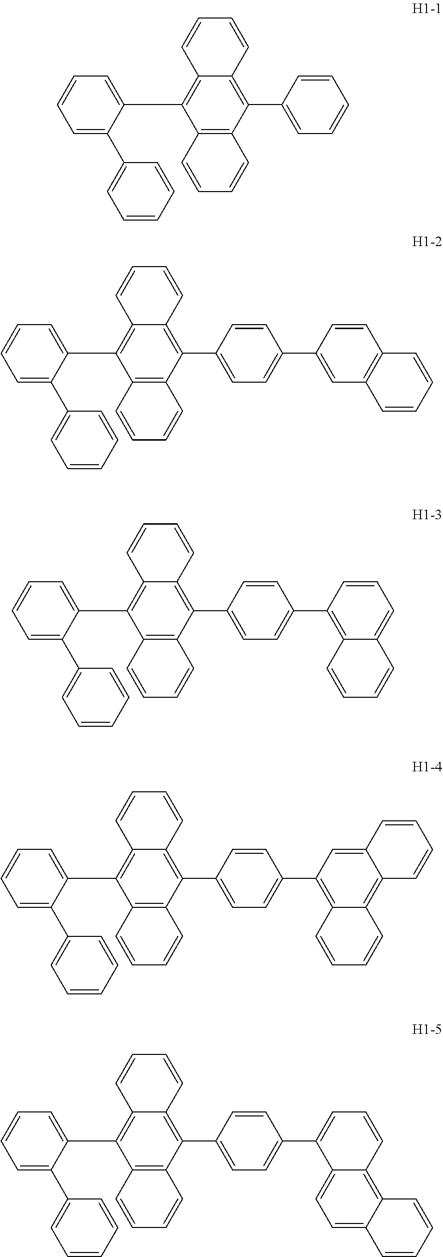

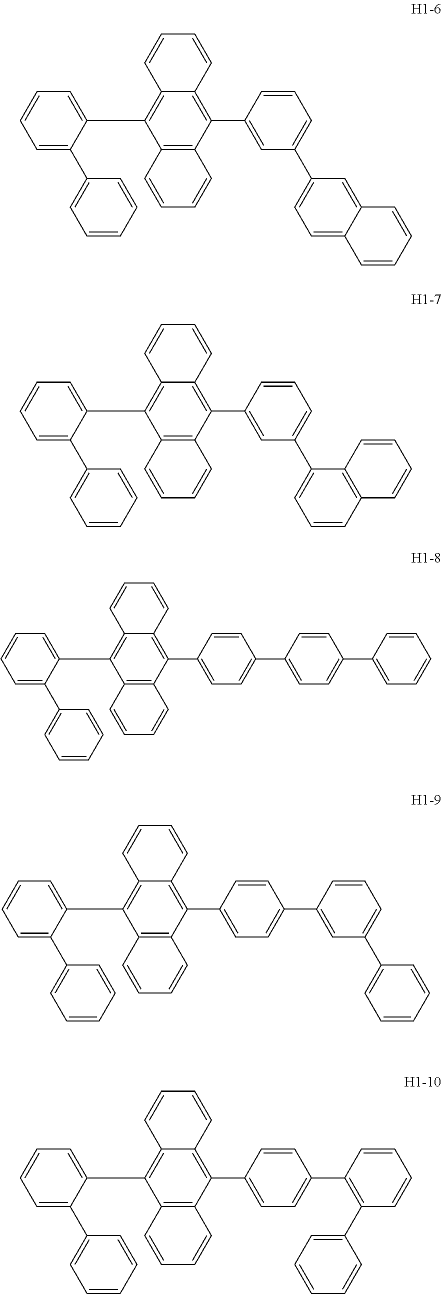

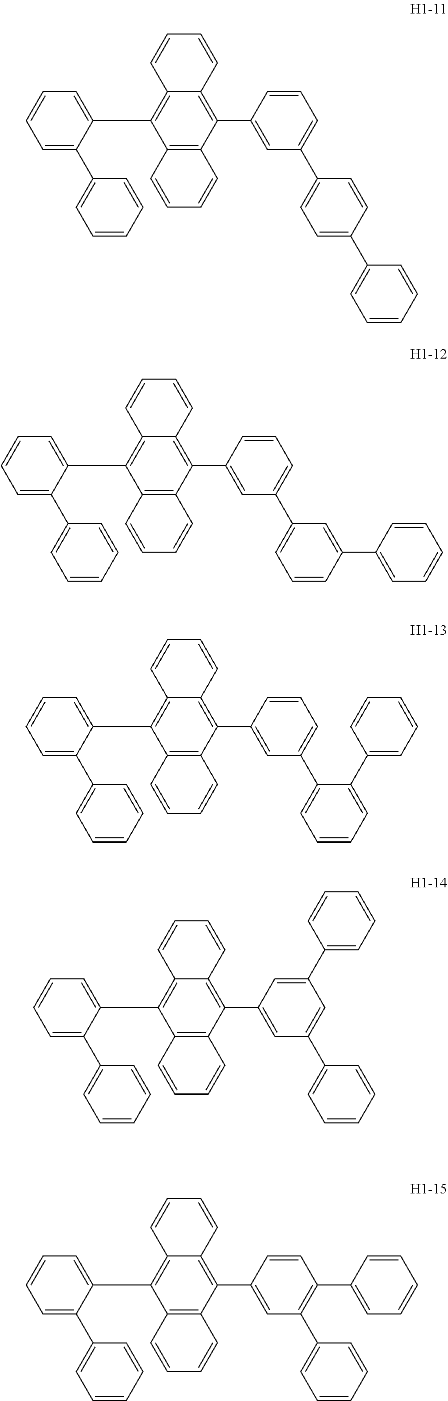

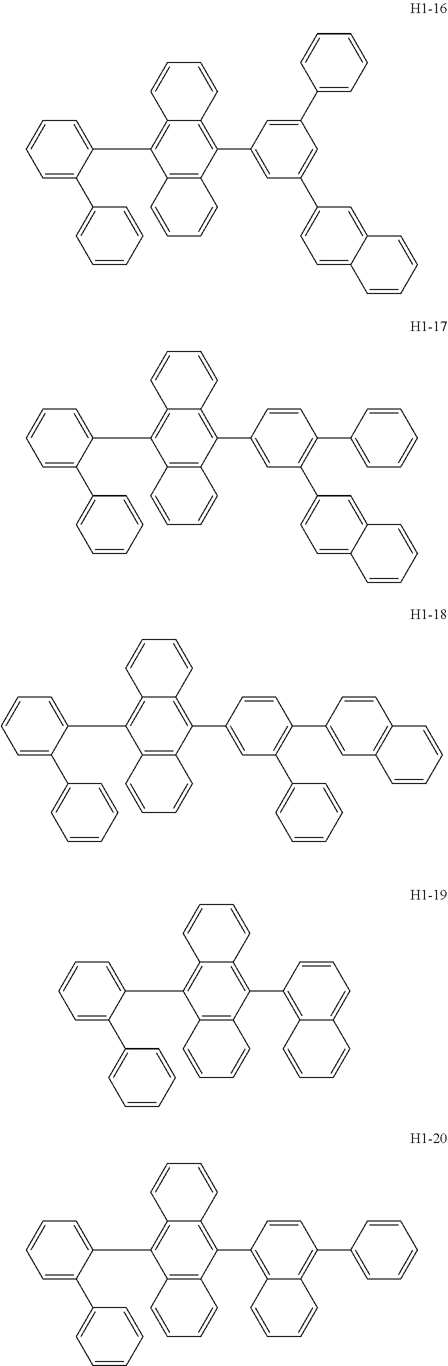

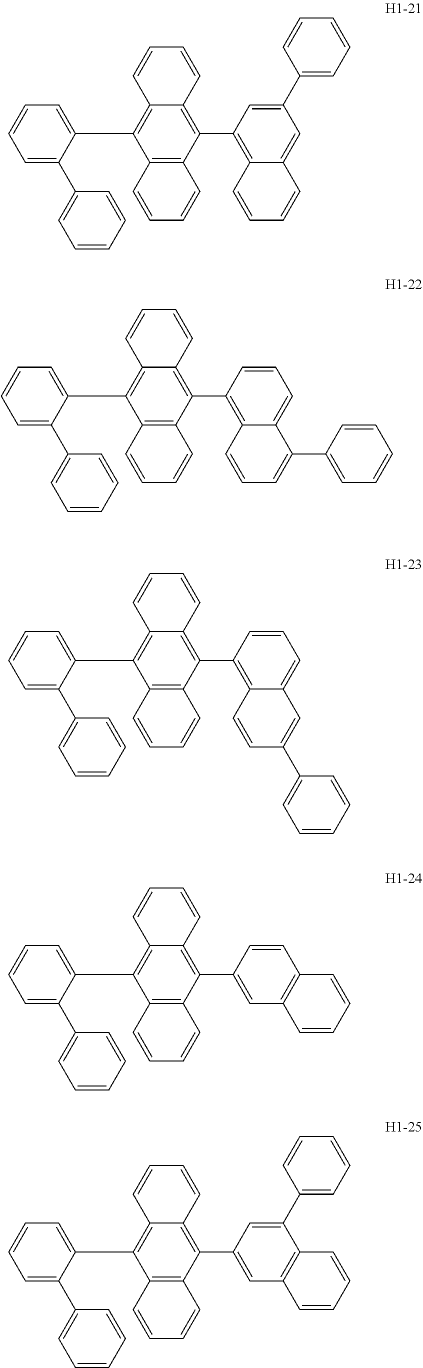

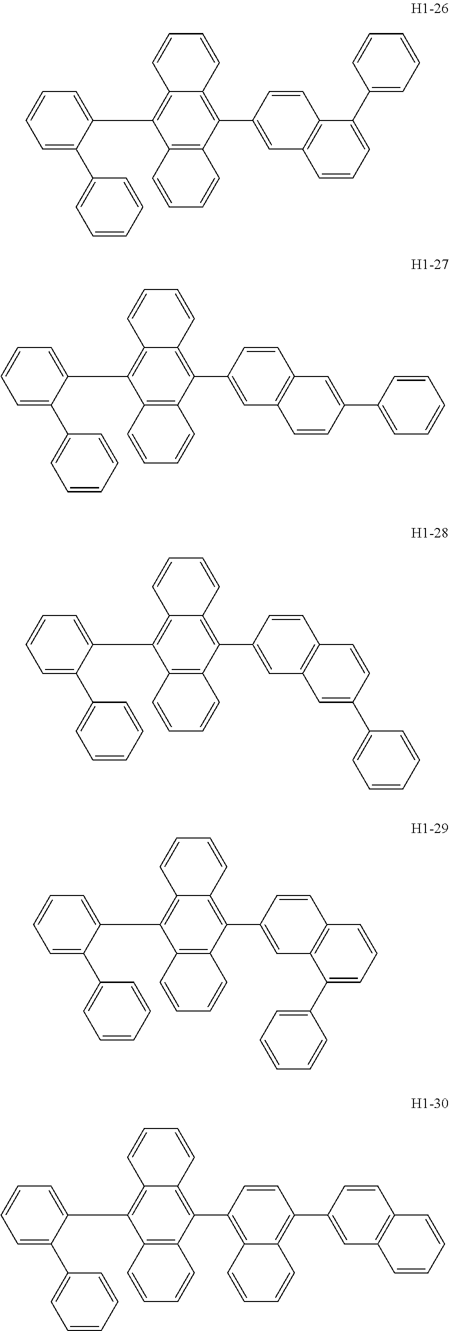

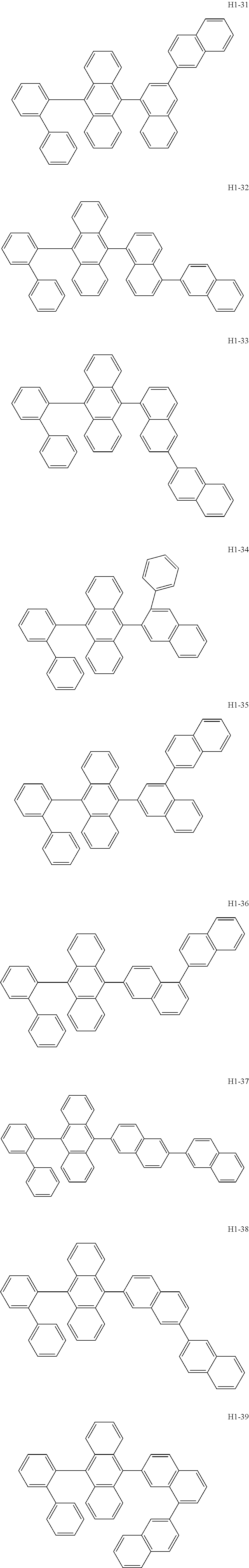

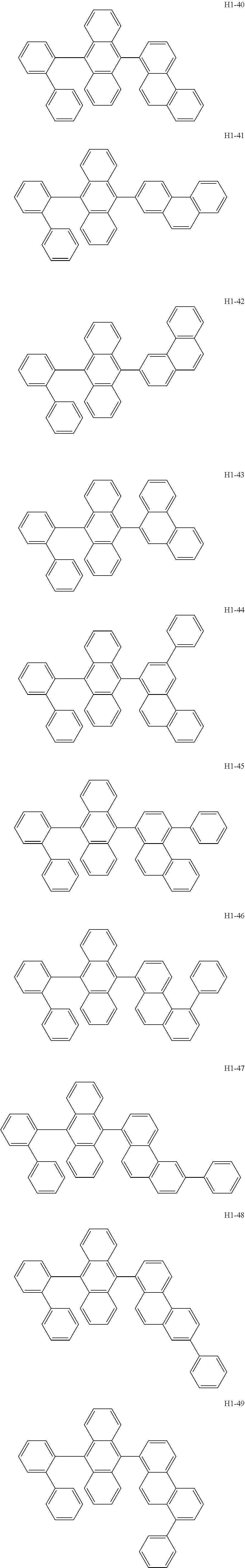

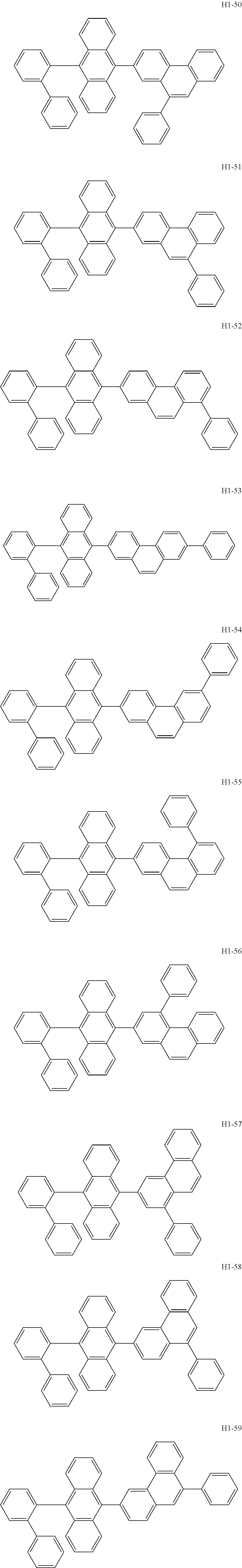

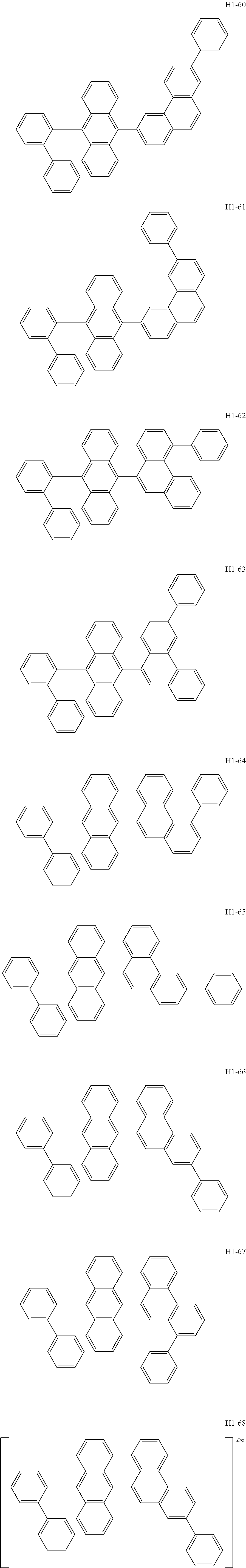

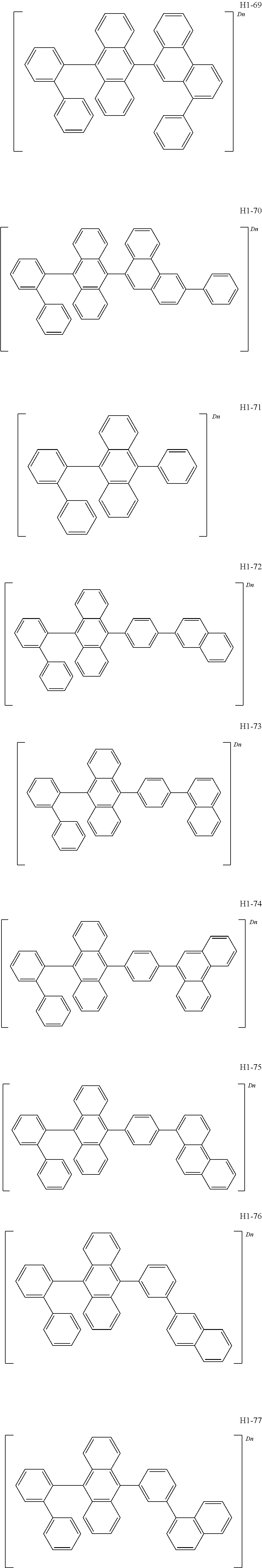

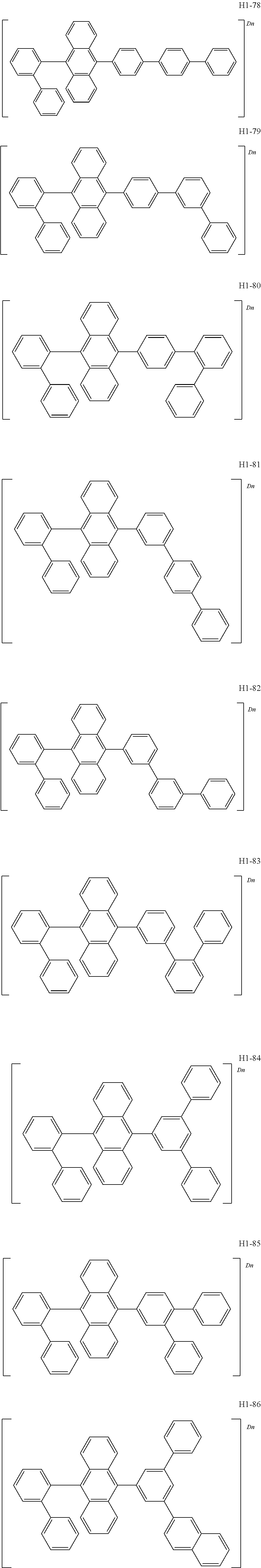

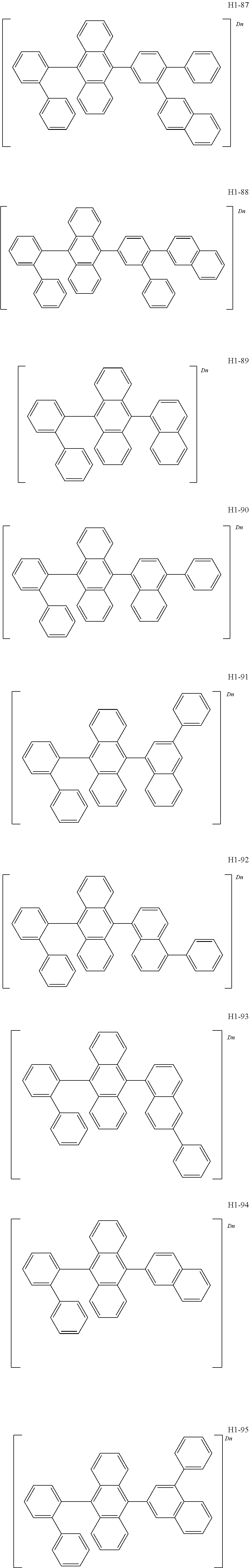

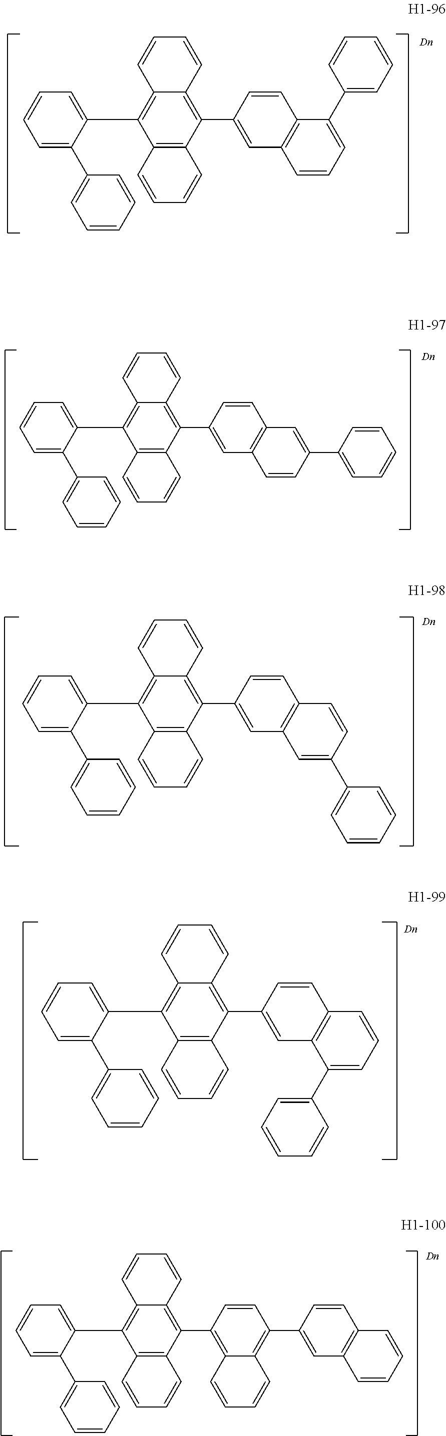

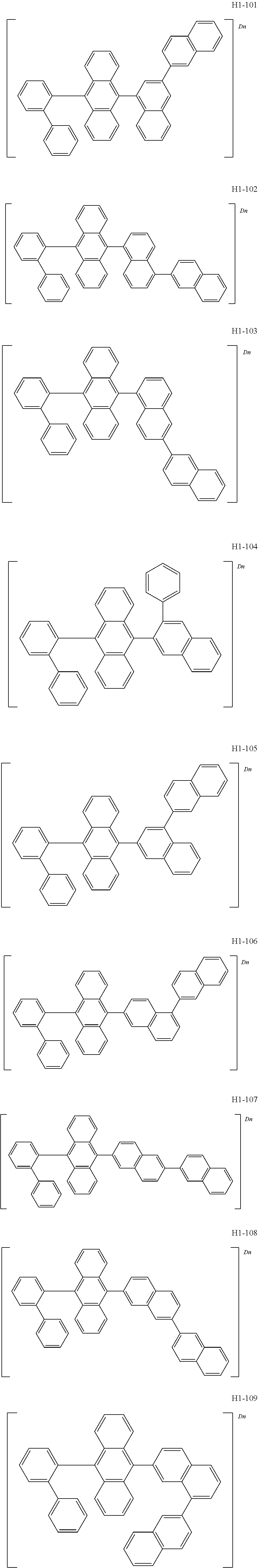

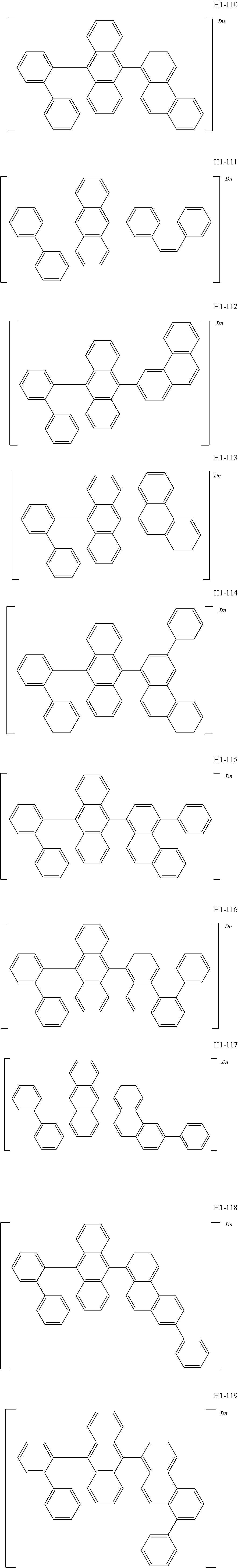

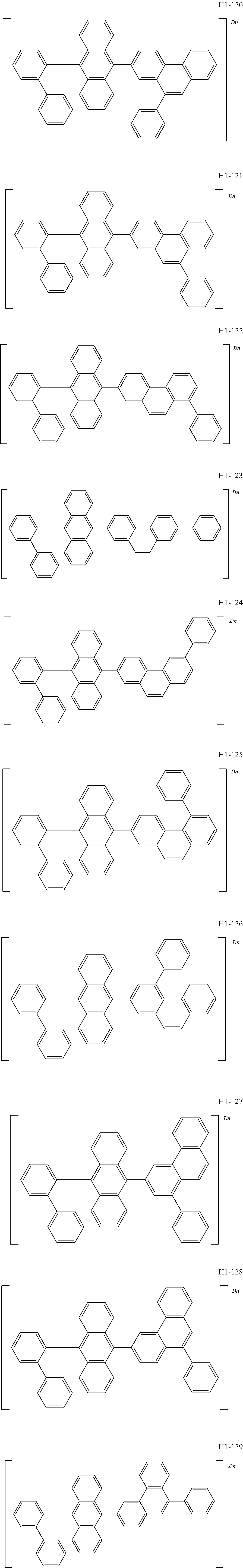

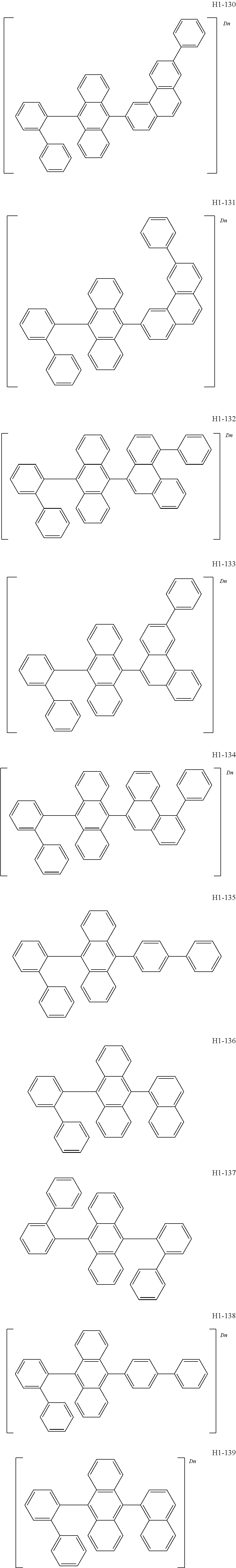

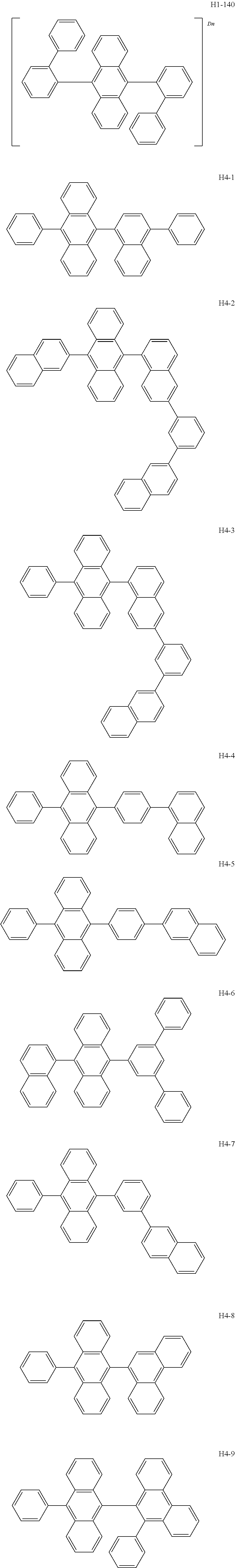

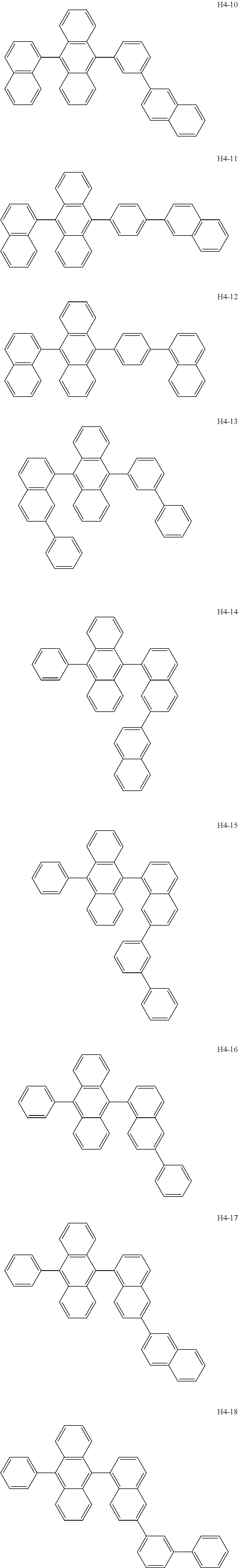

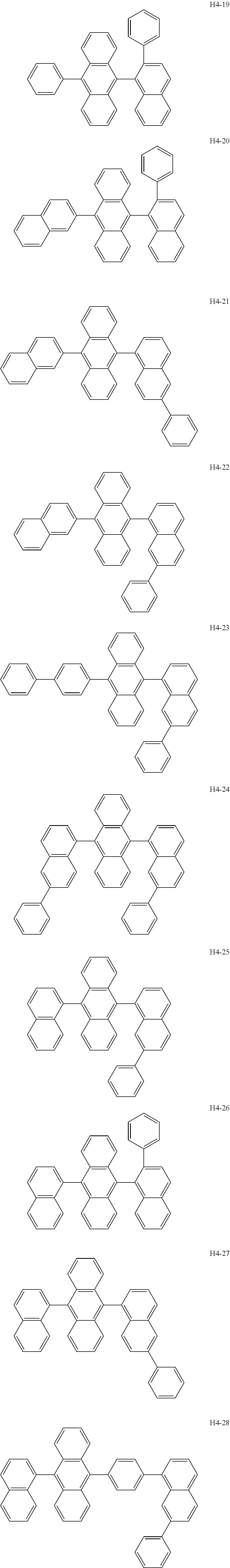

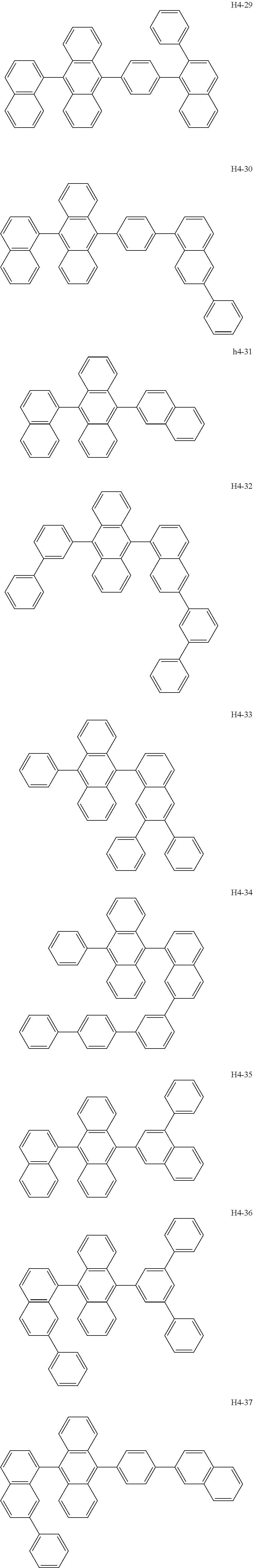

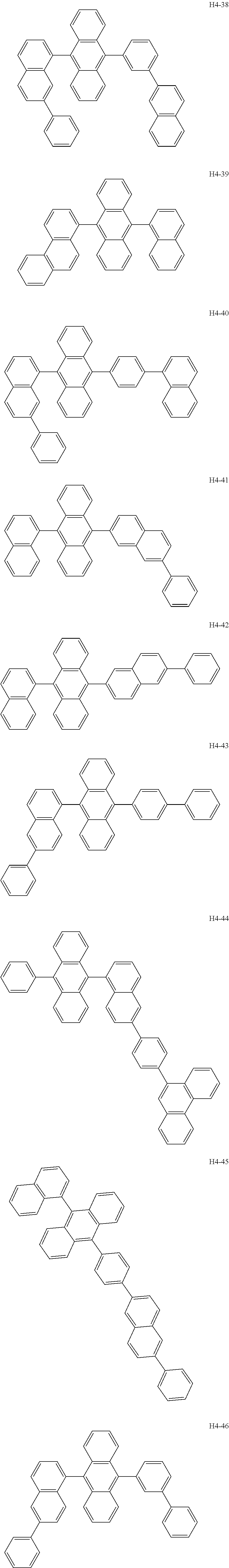

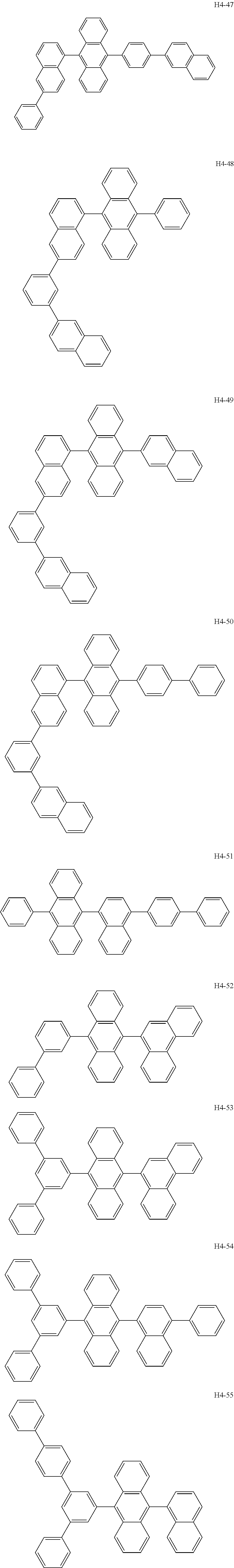

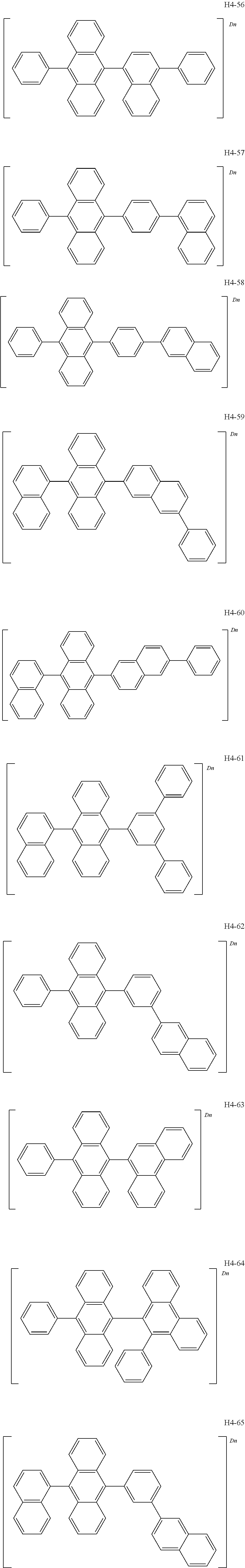

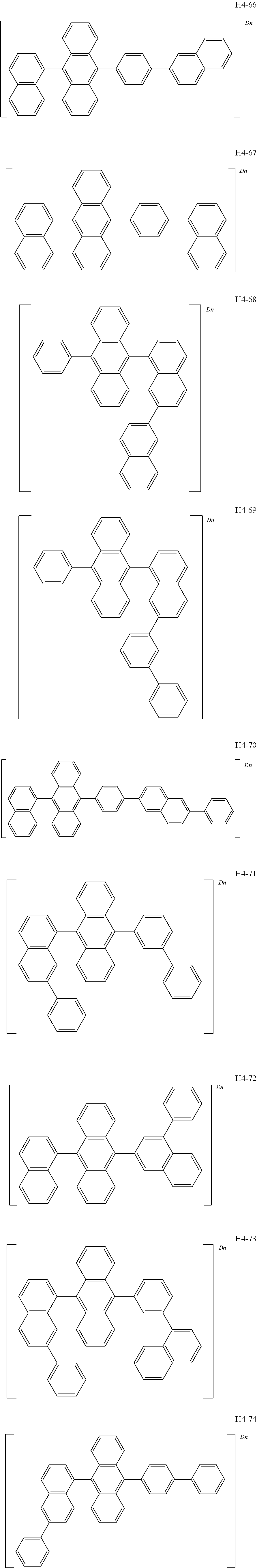

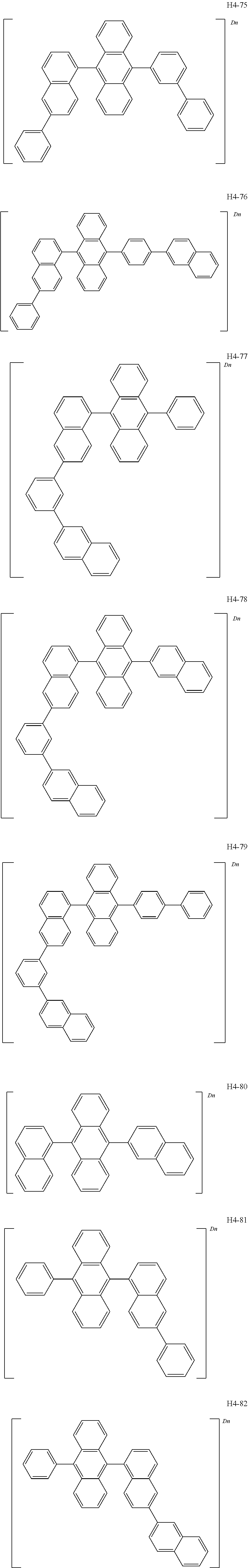

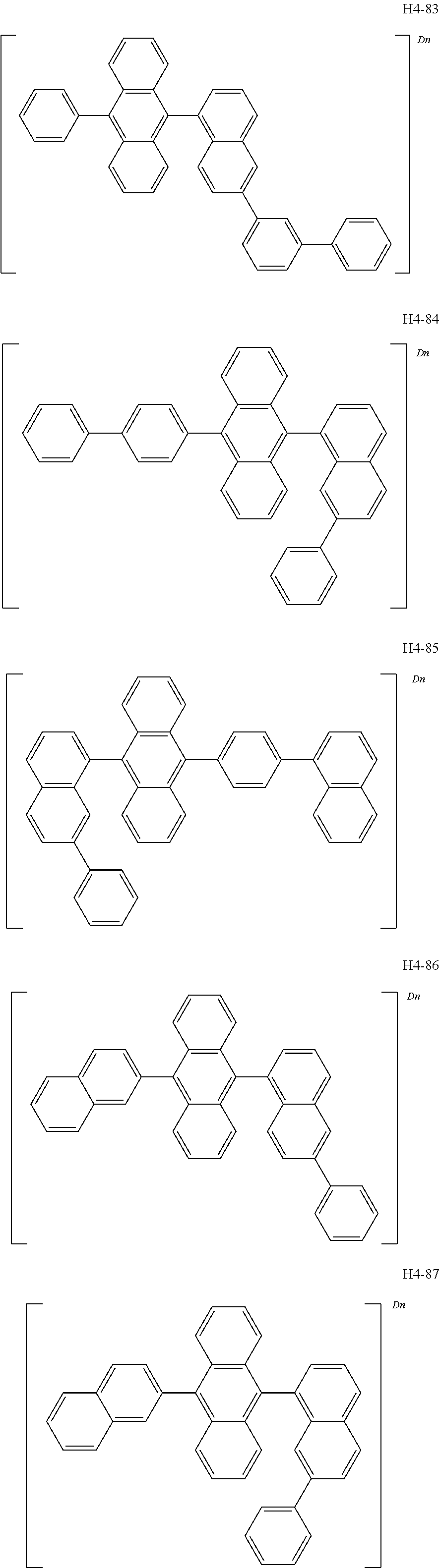

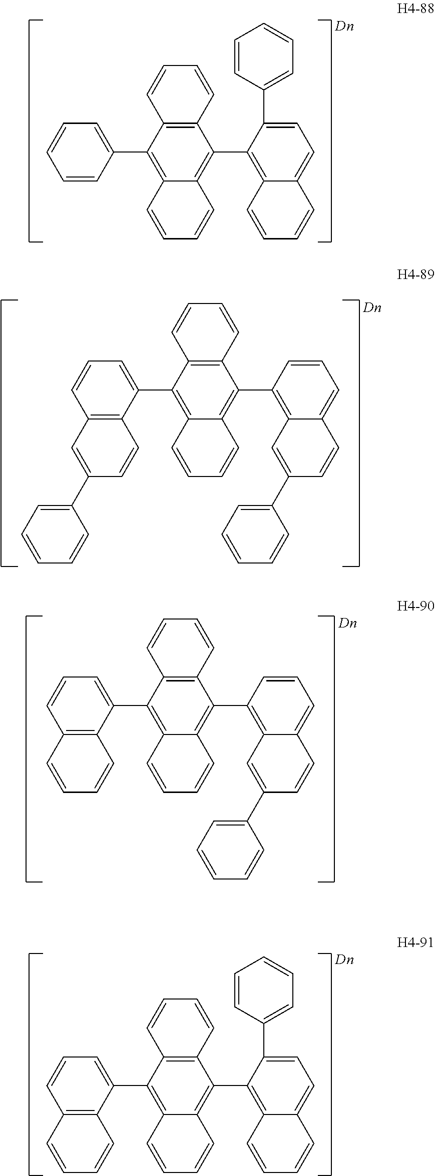

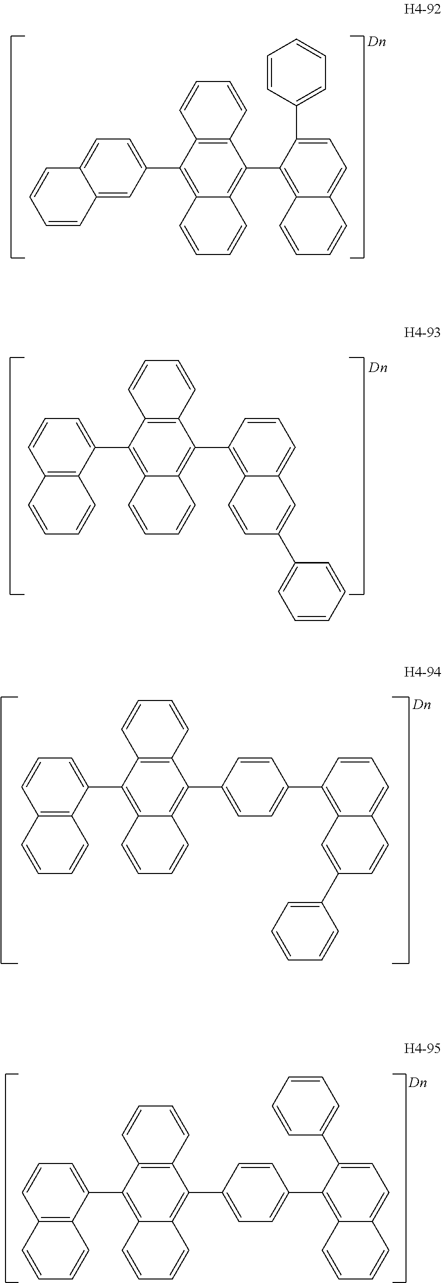

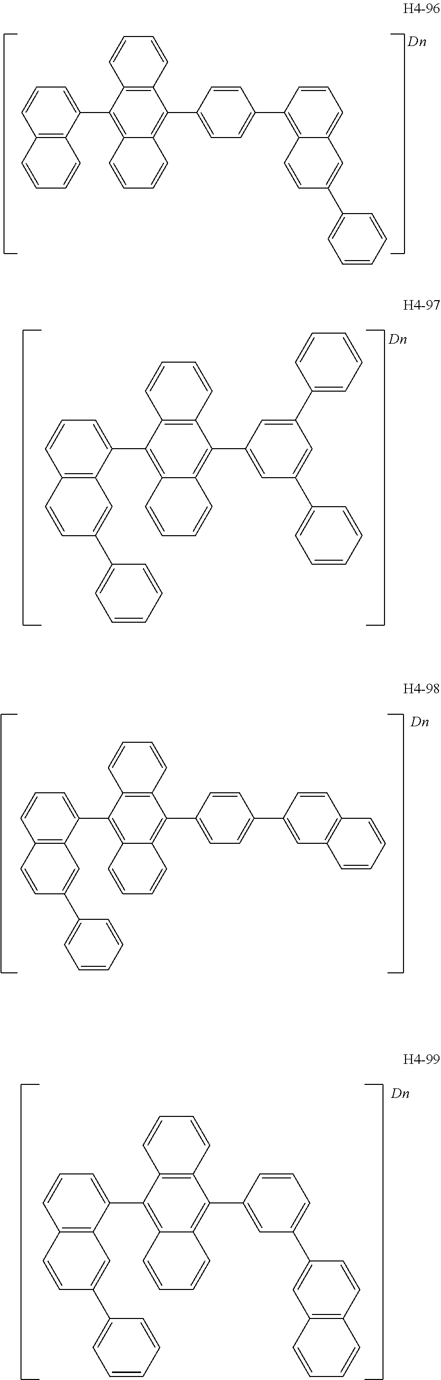

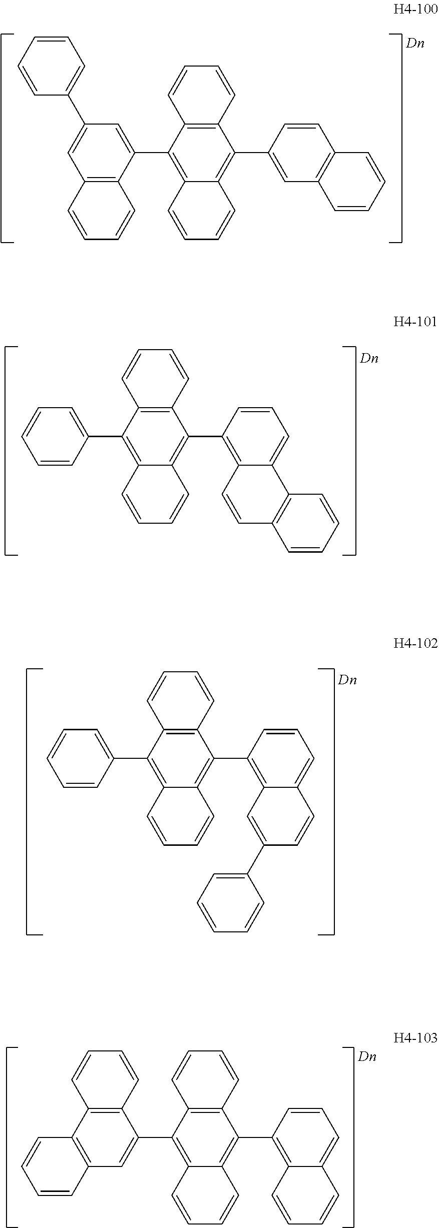

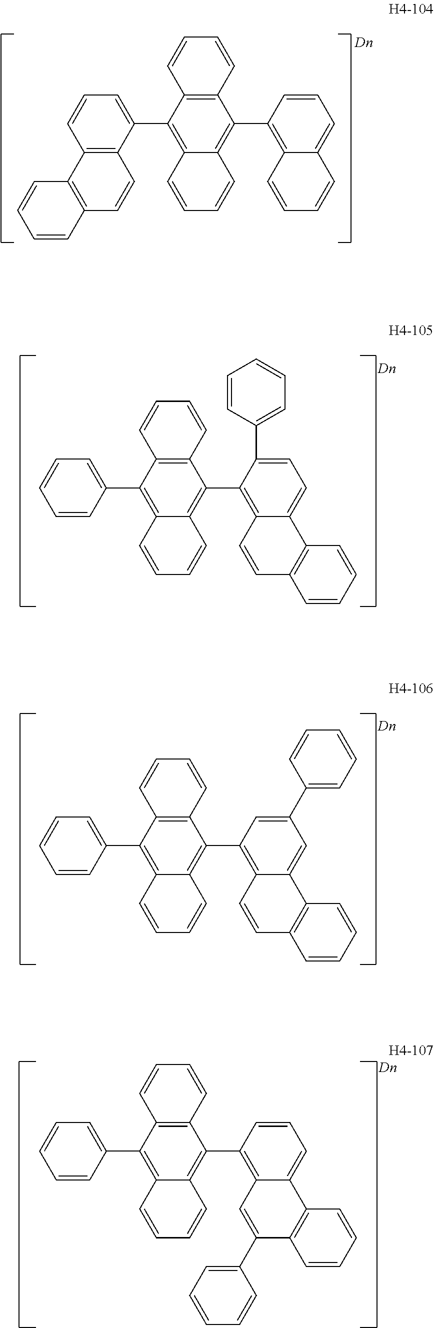

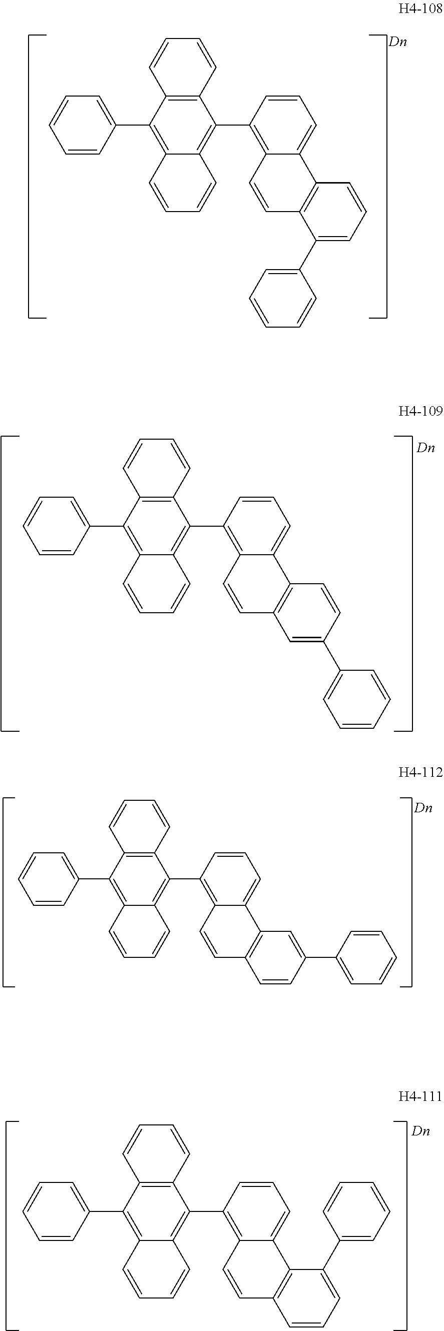

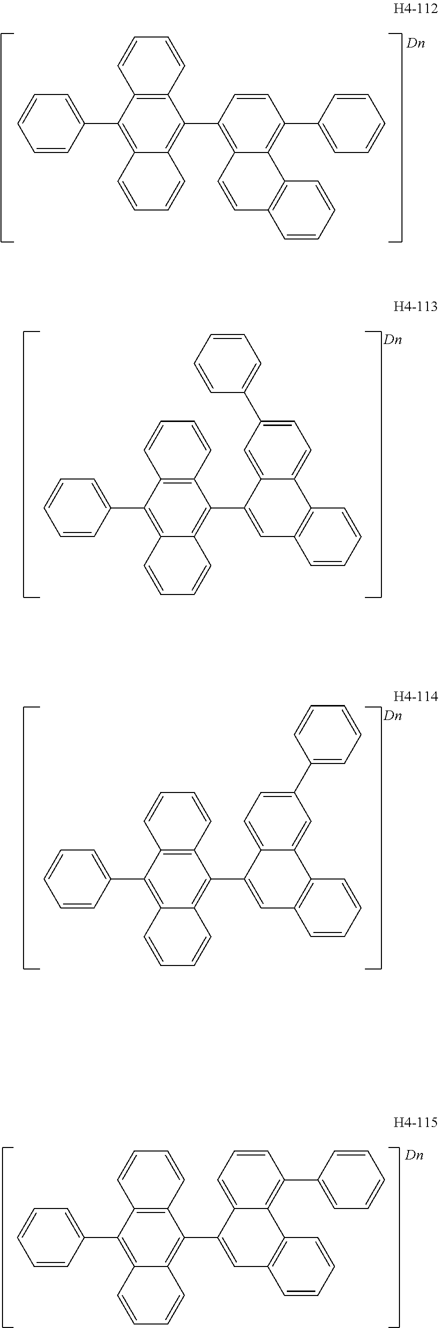

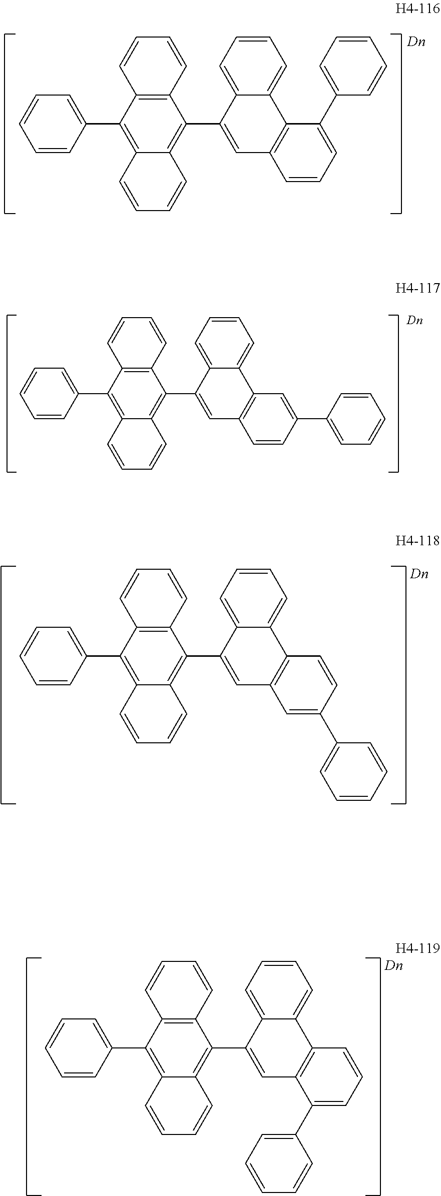

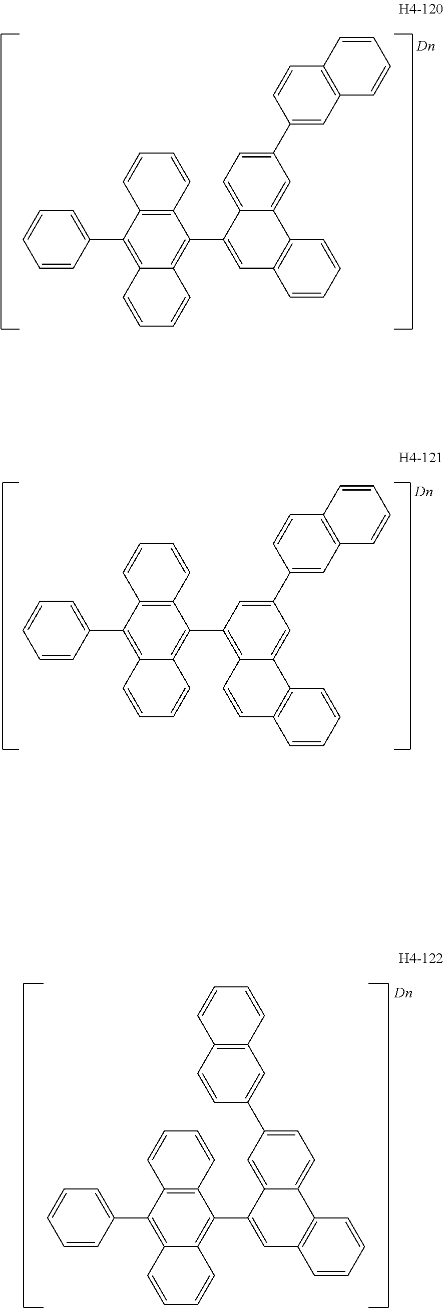

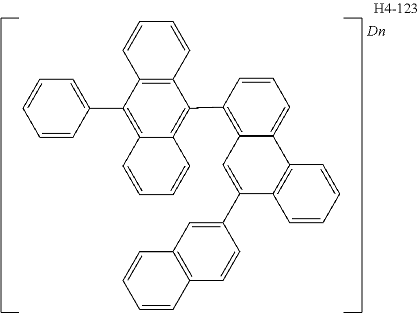

In the above compounds, Dₙ signifies that n hydrogens are replaced with deuterium, and n is an integer from 1 to the maximum number of hydrogens in the compound. Specifically, n is a minimum of 1 and an integer up to the maximum number of hydrogens in the compound. According to one embodiment of the present disclosure, in one compound represented by Formula 2, when deuterium is included, the deuterium substitution rate is preferably about 100% or less, more preferably about 95% or less, still more preferably about 90% or less, and still more preferably about 85% or less of the total number of hydrogens. The compound of Formula 2 substituted with the above deuterium substitution rate can increase the bond dissociation energy according to deuteration, thereby enhancing the stability of the compound, and an organic electroluminescent device comprising the compound can exhibit improved lifetime properties.

Hereinafter, an organic electroluminescent compound according to one embodiment is described.

An organic electroluminescent compound according to one embodiment of the present disclosure is represented by the following Formula 11.

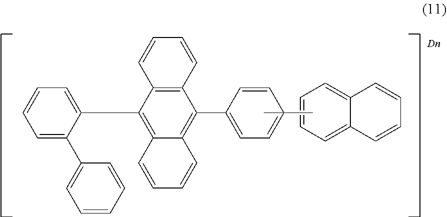

In Formula 11, Dₙ signifies that n hydrogens are replaced with deuterium, and n is an integer from 1 to 28. According to one embodiment of the present disclosure, n is an integer from 8 to 27. According to another embodiment of the present disclosure, the deuterium substitution rate may be preferably about 100% or less, more preferably about 95% or less, still more preferably about 90% or less, and still more preferably about 85% or less of the total number of hydrogens. The compound of Formula 11 substituted with the above deuterium substitution rate can increase the bond dissociation energy according to deuteration, thereby enhancing the stability of the compound, and an organic electroluminescent device comprising the compound can exhibit improved lifetime properties.

The compound represented by Formula 11 may be at least one selected from the group consisting of the following compounds, but is not limited thereto.

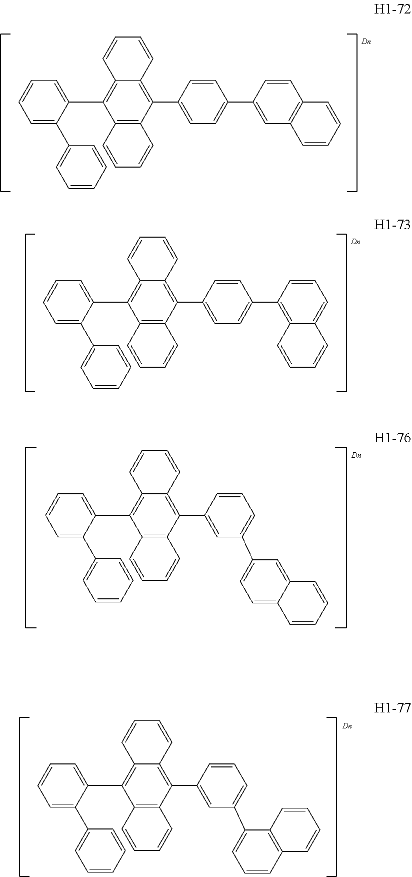

The compounds represented by Formula 1, Formula 2, and Formula 11 according to the present disclosure can be prepared by way of synthetic methods known to one skilled in the art. For example, the compounds of the present disclosure can be prepared by referring to Korean Patent Application Laid-Open No. 2023-0112551, etc., but is not limited thereto. The compounds substituted with deuterium among the compounds of the present disclosure can be prepared by referring to Korean Patent Nos. 10-2283849, 10-1427457, etc., but is not limited thereto.

Hereinafter, an organic electroluminescent device using the aforementioned plurality of host materials or organic electroluminescent compound is described.

According to one embodiment of the present disclosure, an organic electroluminescent device comprises an anode; a cathode; and at least one organic layer between the anode and the cathode, wherein the organic layer comprises at least one light-emitting layer or at least two light-emitting layers, wherein at least one layer of the light-emitting layers comprises a plurality of host materials comprising at least one first host compound represented by Formula 1 and at least one second host compound represented by Formula 2, and wherein the first host compound and the second compound are different from each other. The plurality of host materials may be included in the same organic layer, e.g., a light-emitting layer, or may be included in different light-emitting layers.

According to another embodiment of the present disclosure, an organic electroluminescent device comprises an anode; a cathode; and at least one organic layer between the anode and the cathode, wherein the organic layer comprises at least one light-emitting layer or at least two light-emitting layers, wherein at least one layer of the light-emitting layers comprises the organic electroluminescent compound represented by Formula 11. The compounds may be included in the same organic layer, e.g., a light-emitting layer, or may be included in different light-emitting layers.

According to one embodiment, the organic electroluminescent material of the present disclosure comprises an organic electroluminescent compound represented by Formula 11, and the organic electroluminescent material may be included in an organic layer, e.g., a light-emitting layer.

According to another embodiment of the present disclosure, an organic electroluminescent device comprises an anode; a cathode; and at least two light-emitting layers between the anode and the cathode, wherein at least one layer of the light-emitting layers comprises the compound represented by Formula 1 as a host material(s).

The organic layer comprises a light-emitting layer and may further comprise at least one layer selected from the group consisting of a hole injection layer, a hole transport layer, a hole auxiliary layer, a light-emitting auxiliary layer, an electron transport layer, an electron injection layer, an interlayer, a hole-blocking layer, an electron-blocking layer, and an electron buffer layer. The organic layer may further include an amine-based compound and/or azine-based compound in addition to the light-emitting material of the present disclosure. Specifically, the hole injection layer, the hole transport layer, the hole auxiliary layer, the light-emitting layer, the light-emitting auxiliary layer, or the electron-blocking layer may comprise an amine-based compound, e.g., an arylamine-based compound, a styrylarylamine-based compound, etc., as a hole injection material, a hole transport material, a hole auxiliary material, a light-emitting material, a light-emitting auxiliary material, and an electron-blocking material. In addition, the electron transport layer, the electron injection layer, the electron buffer layer, and the hole-blocking layer may include an azine-based compound as an electron transport material, an electron injection material, an electron buffer material, and a hole-blocking material. Furthermore, the organic layer may further comprise at least one metal selected from the group consisting of metals of Group 1, metals of Group 2, transition metals of the 4th period, transition metals of the 5th period, lanthanides, and organic metals of the d-transition elements of the Periodic Table, or at least one complex compound comprising a metal.

The plurality of host materials or the organic electroluminescent compound according to one embodiment of the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow-green), and B (blue) light-emitting parts, or color conversion material (CCM) method, etc. The plurality of host materials or the organic electroluminescent compound according to one embodiment of the present disclosure may also be used in an organic electroluminescent device comprising a quantum dot (QD).

A hole injection layer, a hole transport layer, an electron-blocking layer, or a combination thereof can be used between the anode and the light-emitting layer. The hole injection layer may be multi-layers in order to lower the hole injection barrier (or hole injection voltage) from the anode to the hole transport layer or the electron-blocking layer, wherein each of the multi-layers may use two compounds simultaneously. In addition, the hole injection layer may be doped with a p-dopant. The electron-blocking layer is located between the hole transport layer (or hole injection layer) and the light-emitting layer, and can prevent light leakage by blocking the overflow of electrons from the light-emitting layer and confining excitons within the light-emitting layer. The hole transport layer or the electron-blocking layer may be multi-layers, wherein a plurality of compounds may be used in each of the multi-layers.

An electron buffer layer, a hole-blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole-blocking layer is located between the electron transport layer (or electron injection layer) and the light-emitting layer, and can improve the probability of recombination of electrons and holes in the light-emitting layer by preventing holes from reaching the cathode. The hole-blocking layer or the electron transport layer may be multi-layers, wherein a plurality of compounds may be used in each of the multi-layers. In addition, the electron injection layer may be doped with an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the injection and/or transport of hole, or for preventing the overflow of electrons. When the light-emitting auxiliary layer is placed between the cathode and the light-emitting layer, it can be used for promoting the injection and/or transport of electron, or for preventing the overflow of holes. In addition, the hole auxiliary layer may be placed between the hole transport layer (or hole injection layer) and the light-emitting layer, and may be effective to promote or block the hole transport rate (or hole injection rate), thereby enabling the charge balance to be controlled. When an organic electroluminescent device includes two or more hole transport layers, the hole transport layer, which is further included, may be used as a hole auxiliary layer or an electron-blocking layer. The light-emitting auxiliary layer, the hole auxiliary layer, or the electron-blocking layer may be provided to improve the efficiency and/or lifetime properties of the organic electroluminescent device.

In the organic electroluminescent device of the present disclosure, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum may be placed on an anode surface of an electroluminescent medium layer, and a metal halide layer or a metal oxide layer may be placed on a cathode surface of an electroluminescent medium layer. Such a surface layer provides operation stability for the organic electroluminescent device. For example, the chalcogenide includes SiO_{X} (1≤X≤2), AlO_{X} (1≤X≤1.5), SiON, SiAlON, etc.; the metal halide includes LiF, MgF₂, CaF₂, a rare earth metal fluoride, etc.; and the metal oxide includes Cs₂O, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this way, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Specifically, the oxidative dopant includes various Lewis acids and acceptor compounds; and the reductive dopant includes alkali metals, alkali metal compounds, alkaline earth metals, rare earth metals, and mixtures thereof. The reductive dopant layer may be employed as a charge-generating layer to produce an organic electroluminescent device having two or more light-emitting layers and emitting white light.

An organic electroluminescent device according to one embodiment may further include one or more dopants in the light-emitting layer.

The dopant comprised in the organic electroluminescent device of the present disclosure may be at least one phosphorescent or fluorescent dopant, and preferably, a fluorescent dopant. The phosphorescent dopant material is not particularly limited, but may be a complex compound of a metal selected from the group consisting of iridium (Ir), osmium (Os), copper (Cu), and platinum (Pt), preferably an ortho-metallated complex compound of a metal selected from the group consisting of iridium (Ir), osmium (Os), copper (Cu), and platinum (Pt), and more preferably an ortho-metallated iridium complex compound.

A compound represented by the following Formula D may be used as a dopant included in the organic electroluminescent device of the present disclosure, but is not limited thereto.

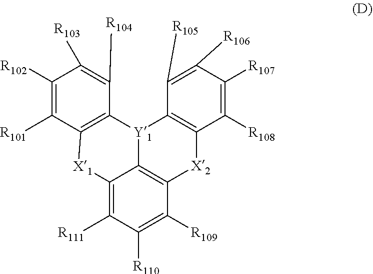

In Formula D,
R₁₀₁ to R₁₁₁ each independently represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅), or may be linked to the adjacent substituent to form a ring(s);
Y′₁ represents B;
X′₁ and X′₂ each independently represent NR′;
R′ each independently represents hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or -L′₄-N—(Ar′₄)(Ar′₅); or may be linked to at least one of R₁₀₁, R₁₀₈, R₁₀₉, and R₁₁₁ to form a ring(s);
L′₄ each independently represents a single bond, a substituted or unsubstituted (C₆-C₃₀)arylene, or a substituted or unsubstituted (3- to 30-membered) heteroarylene; and
Ar′₄ and Ar′₅ each independently represent hydrogen, a substituted or unsubstituted (C₁-C₃₀)alkyl, a substituted or unsubstituted (C₂-C₃₀)alkenyl, a substituted or unsubstituted fused ring group of a (C₃-C₃₀) aliphatic ring(s) and a (C₆-C₃₀) aromatic ring(s), a substituted or unsubstituted (C₆-C₃₀)aryl, or a substituted or unsubstituted (3- to 30-membered)heteroaryl.

Preferably, R₁₀₁ to R₁₁₁ each independently represent hydrogen, deuterium, a substituted or unsubstituted (C₁-C₂₀)alkyl, a substituted or unsubstituted (C₆-C₂₅)aryl, a substituted or unsubstituted (5- to 20-membered)heteroaryl, or -L′₄-N—(Ar′₄)(Ar′₅), or may be linked to the adjacent substituent to form a ring(s).

More preferably, R₁₀₁ to R₁₁₁ each independently represent hydrogen; deuterium; an unsubstituted (C₁-C₁₀)alkyl; a (C₆-C₁₈)aryl unsubstituted or substituted with at least one of a (C₁-C₁₀)alkyl(s), a (13- to 18-membered) hetero-aryl(s), and a di(C₆-C₁₈)arylamino(s); a (5- to 18-membered)heteroaryl unsubstituted or substituted with at least one (C₁-C₁₀)alkyl(s); or -L′₄-N—(Ar′₄)(Ar′₅); or may be linked to the adjacent substituent to form a ring(s). For example, R₁₀₁ to R₁₁₁ each independently may be hydrogen, methyl, tert-butyl, a substituted or unsubstituted phenyl, a biphenyl, a terphenyl, a triphenylenyl, a carbazolyl, a phenoxazinyl, a phenothiazinyl, a dimethylacridinyl, a dimethylxanthenyl, a diphenylamino unsubstituted or substituted with at least one of a methyl(s) and a diphenylamino(s), a phenylnaphthylamino, a dibiphenylamino, a phenylamino substituted with a phenylcarbazolyl(s) or a dibenzofuranyl(s), or a (17- to 21-membered)heteroaryl substituted with at least one of a methyl(s) and a phenyl(s), or may be linked to adjacent substituent to form a benzene ring, an indole ring substituted with at least one of a phenyl(s) and a diphenylamino(s), a benzofuran ring, a benzothiophene ring, or a 19-membered hetero ring substituted with a methyl(s). The substituent of the above substituted phenyl may be at least one of a methyl(s), a carbazolyl(s), a dibenzofuranyl(s), a diphenylamino(s), a phenoxazinyl(s), a phenothiazinyl(s), and a dimethylacridinyl(s).

The specific examples of the dopant compound are as follows, but are not limited thereto.

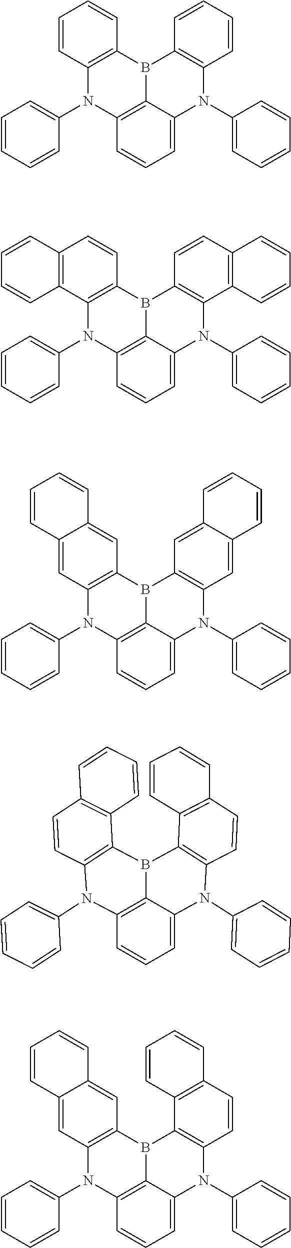 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1]

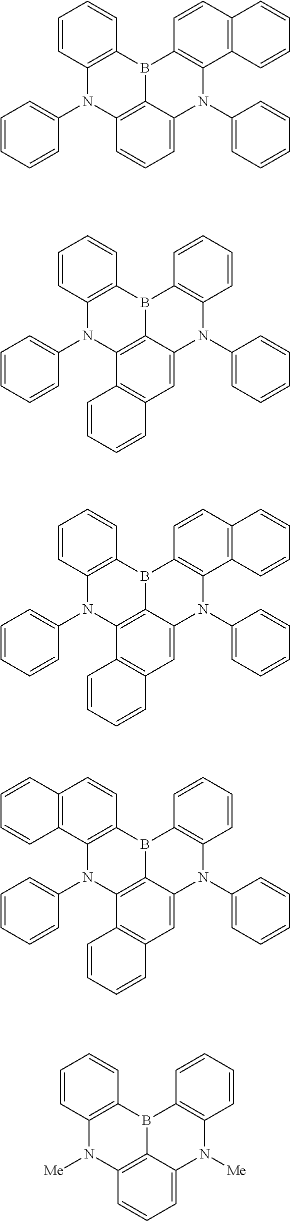 [SMILES: c1ccc(N2c3ccccc3B3c4ccc5ccccc5c4N(c4ccccc4)c4cccc2c43)cc1 ; c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32]

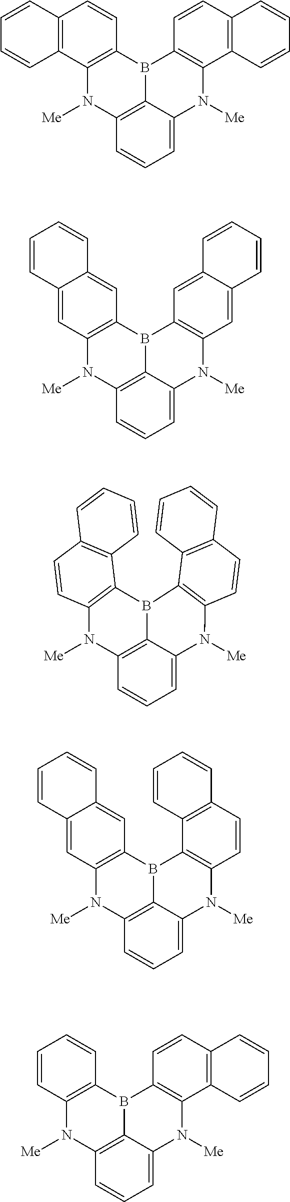 [SMILES: CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

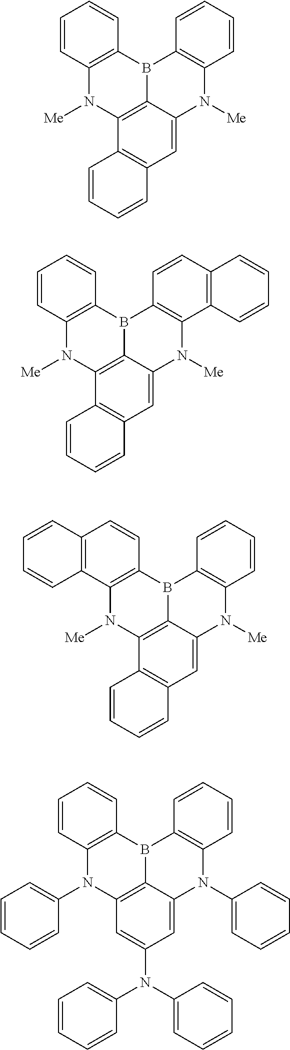 [SMILES: CN1c2ccccc2B2c3ccccc3N(C)c3c2c1cc1ccccc31 ; CN1c2cc3ccccc3c3c2B(c2ccccc2N3C)c2ccc3ccccc3c21 ; CN1c2ccccc2B2c3ccc4ccccc4c3N(C)c3c2c1cc1ccccc31 ; c1ccc(N(c2ccccc2)c2cc3c4c(c2)N(c2ccccc2)c2ccccc2B4c2ccccc2N3c2ccccc2)cc1]

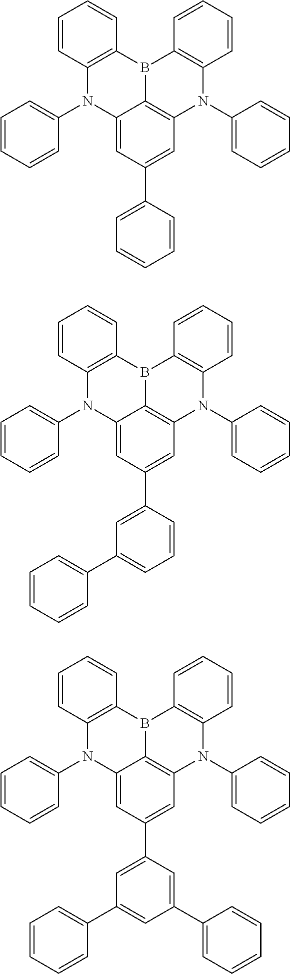

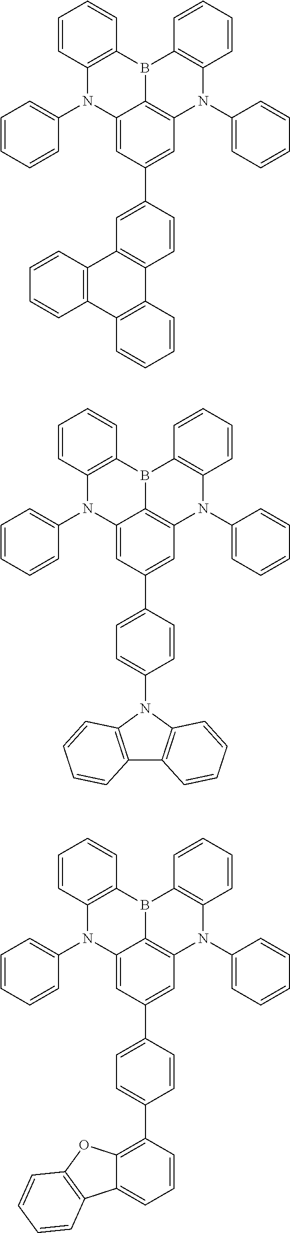 [SMILES: c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1]

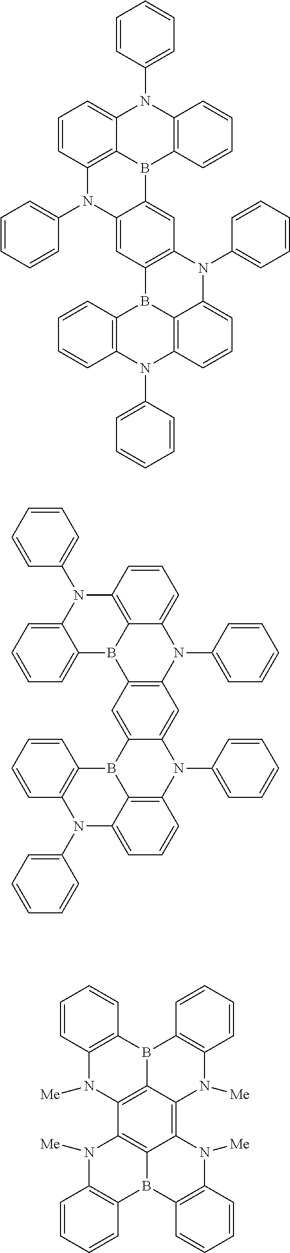 [SMILES: c1ccc(N2c3ccccc3B3c4cc5c(cc4N(c4ccccc4)c4cccc2c43)B2c3ccccc3N(c3ccccc3)c3cccc(c32)N5c2ccccc2)cc1 ; c1ccc(N2c3ccccc3B3c4cc5c(cc4N(c4ccccc4)c4cccc2c43)N(c2ccccc2)c2cccc3c2B5c2ccccc2N3c2ccccc2)cc1 ; CN1c2ccccc2B2c3ccccc3N(C)c3c2c1c1c2c3N(C)c3ccccc3B2c2ccccc2N1C]

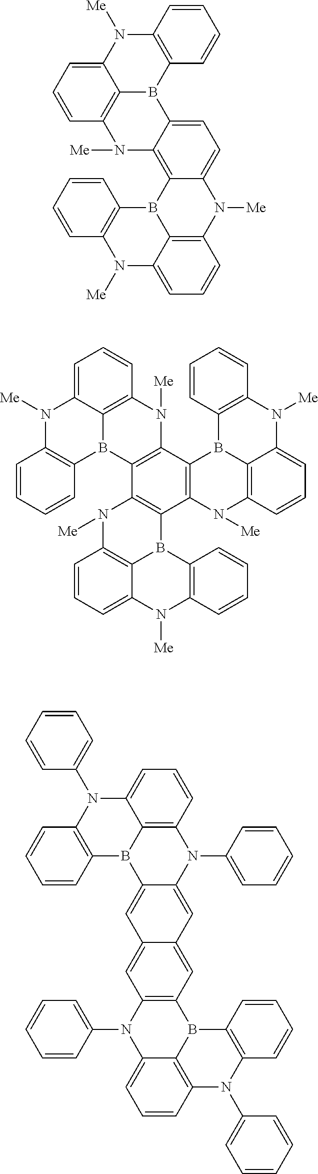 [SMILES: CN1c2ccccc2B2c3c1cccc3N(C)c1ccc3c(c12)N(C)c1cccc2c1B3c1ccccc1N2C ; CN1c2ccccc2B2c3c1cccc3N(C)c1c3c(c4c(c12)N(C)c1cccc2c1B4c1ccccc1N2C)N(C)c1cccc2c1B3c1ccccc1N2C ; c1ccc(N2c3ccccc3B3c4cc5cc6c(cc5cc4N(c4ccccc4)c4cccc2c43)B2c3ccccc3N(c3ccccc3)c3cccc(c32)N6c2ccccc2)cc1]

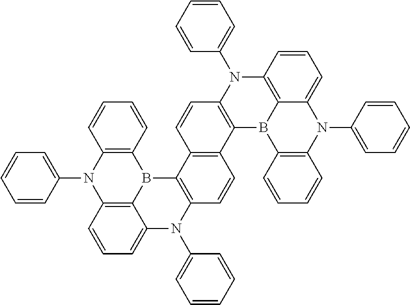 [SMILES: c1ccc(N2c3ccccc3B3c4c2cccc4N(c2ccccc2)c2ccc4c5c(ccc4c23)N(c2ccccc2)c2cccc3c2B5c2ccccc2N3c2ccccc2)cc1]

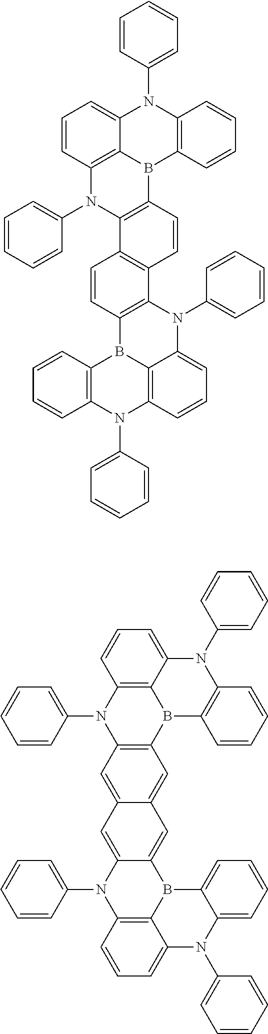 [SMILES: c1ccc(N2c3ccccc3B3c4ccc5c6c(ccc5c4N(c4ccccc4)c4cccc2c43)B2c3ccccc3N(c3ccccc3)c3cccc(c32)N6c2ccccc2)cc1 ; c1ccc(N2c3ccccc3B3c4cc5cc6c(cc5cc4N(c4ccccc4)c4cccc2c43)N(c2ccccc2)c2cccc3c2B6c2ccccc2N3c2ccccc2)cc1]

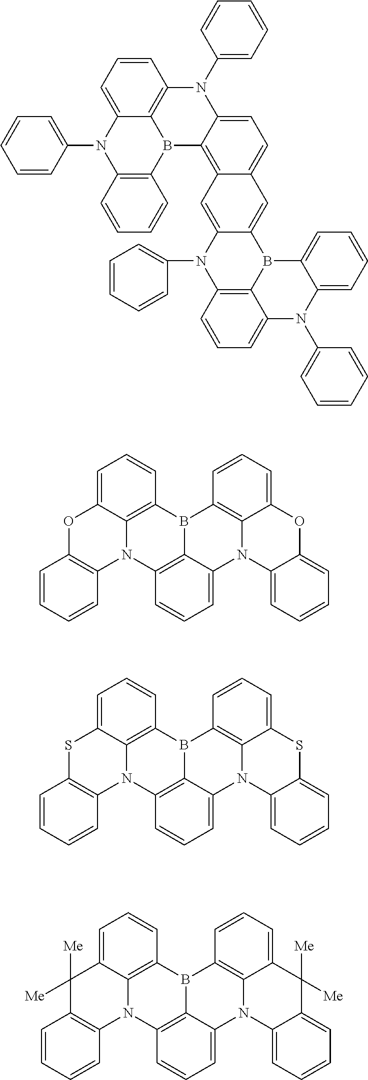 [SMILES: c1ccc(N2c3ccccc3B3c4cc5ccc6c(c5cc4N(c4ccccc4)c4cccc2c43)B2c3ccccc3N(c3ccccc3)c3cccc(c32)N6c2ccccc2)cc1 ; c1ccc2c(c1)Oc1cccc3c1N2c1cccc2c1B3c1cccc3c1N2c1ccccc1O3 ; c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C]

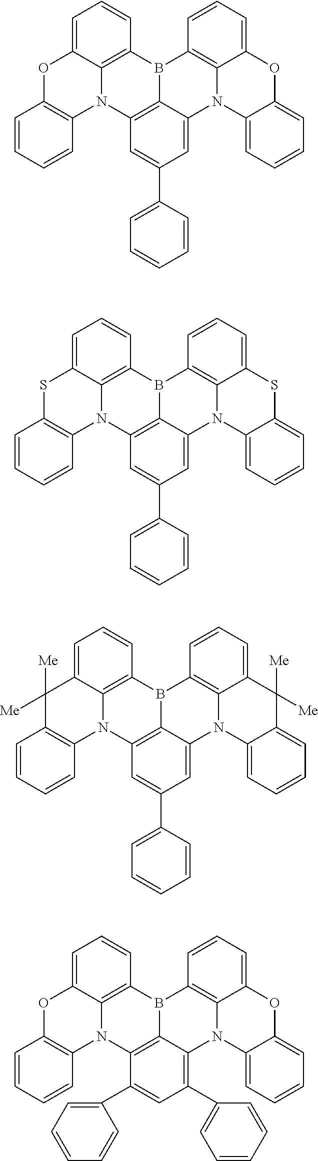 [SMILES: c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C ; c1ccc(-c2cc(-c3ccccc3)c3c4c2N2c5ccccc5Oc5cccc(c52)B4c2cccc4c2N3c2ccccc2O4)cc1]

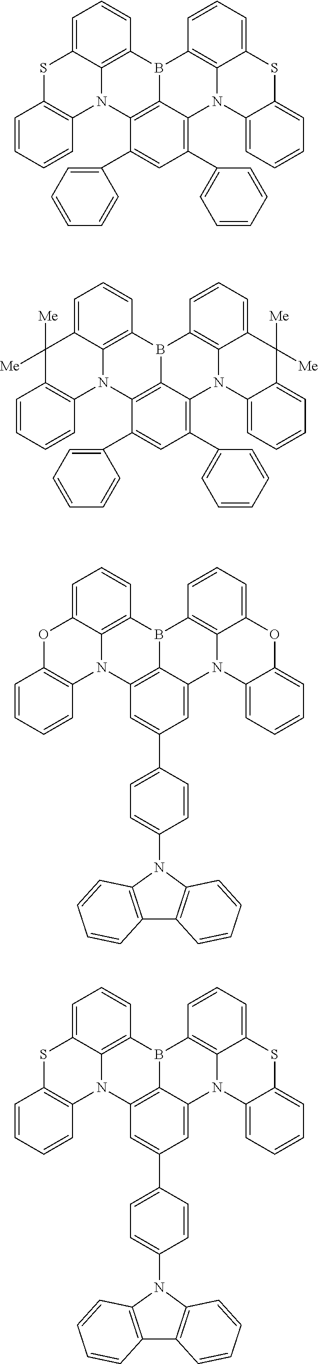 [SMILES: c1ccc(-c2cc(-c3ccccc3)c3c4c2N2c5ccccc5Sc5cccc(c52)B4c2cccc4c2N3c2ccccc2S4)cc1 ; CC1(C)c2ccccc2N2c3c(cccc31)B1c3cccc4c3N(c3ccccc3C4(C)C)c3c(-c4ccccc4)cc(-c4ccccc4)c2c31 ; c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc2c(c1)Sc1cccc3c1N2c1cc(-c2ccc(-n4c5ccccc5c5ccccc54)cc2)cc2c1B3c1cccc3c1N2c1ccccc1S3]

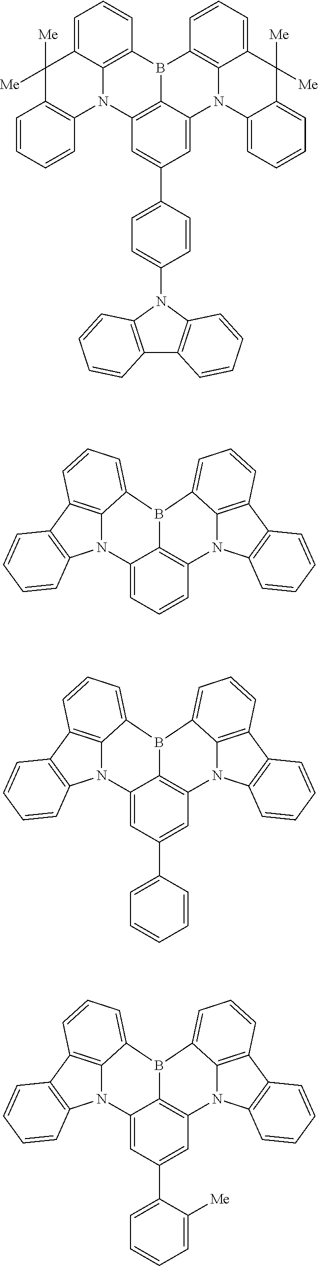 [SMILES: CC1(C)c2ccccc2N2c3cc(-c4ccc(-n5c6ccccc6c6ccccc65)cc4)cc4c3B(c3cccc1c32)c1cccc2c1N4c1ccccc1C2(C)C ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

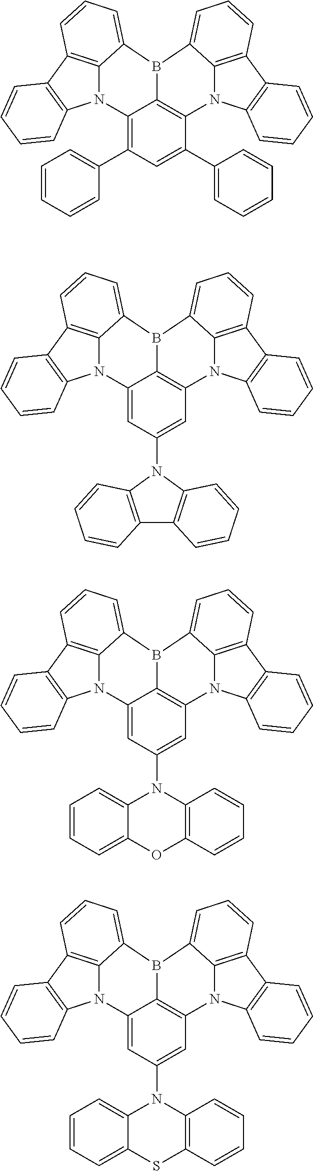 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13]

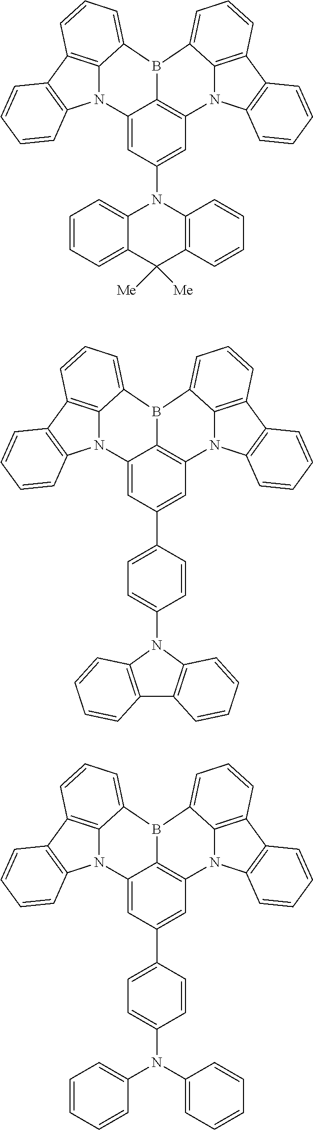 [SMILES: CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1]

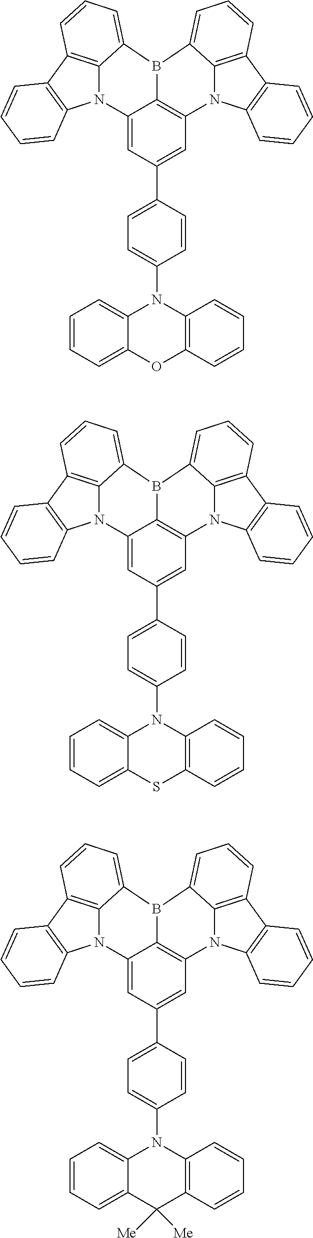 [SMILES: c1ccc2c(c1)Oc1ccccc1N2c1ccc(-c2cc3c4c(c2)-n2c5ccccc5c5cccc(c52)B4c2cccc4c5ccccc5n-3c24)cc1 ; c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

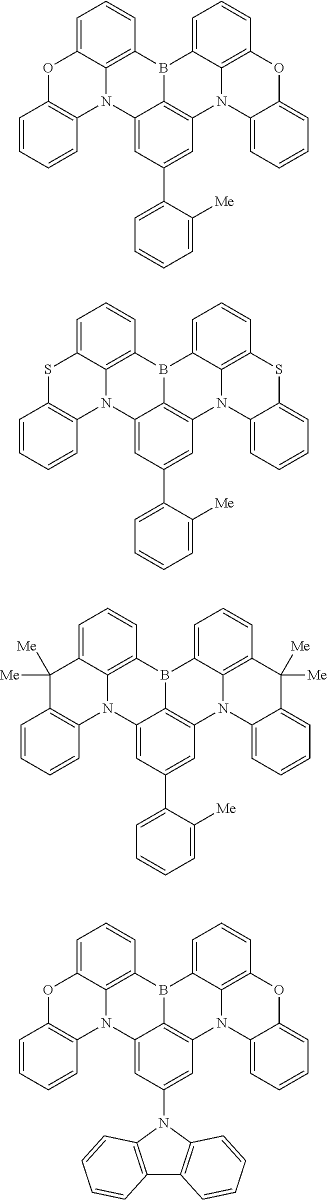 [SMILES: Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C ; c1ccc2c(c1)Oc1cccc3c1N2c1cc(-n2c4ccccc4c4ccccc42)cc2c1B3c1cccc3c1N2c1ccccc1O3]

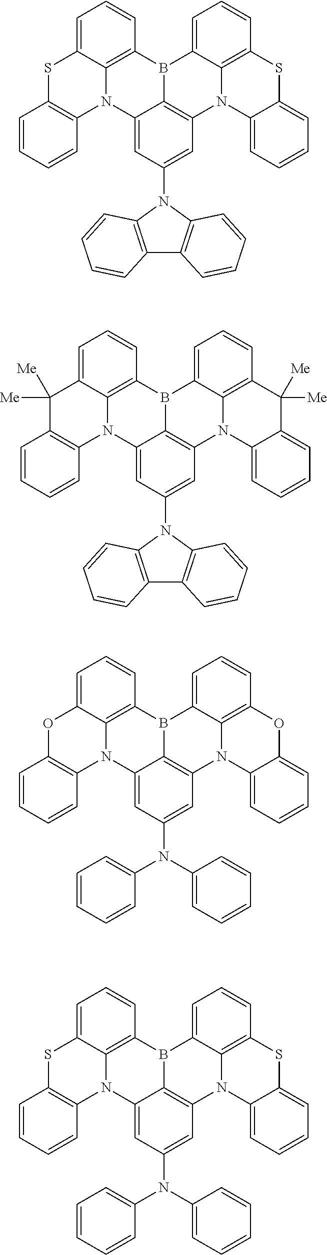 [SMILES: c1ccc2c(c1)Sc1cccc3c1N2c1cc(-n2c4ccccc4c4ccccc42)cc2c1B3c1cccc3c1N2c1ccccc1S3 ; CC1(C)c2ccccc2N2c3cc(-n4c5ccccc5c5ccccc54)cc4c3B(c3cccc1c32)c1cccc2c1N4c1ccccc1C2(C)C ; c1ccc(N(c2ccccc2)c2cc3c4c(c2)N2c5ccccc5Oc5cccc(c52)B4c2cccc4c2N3c2ccccc2O4)cc1 ; c1ccc(N(c2ccccc2)c2cc3c4c(c2)N2c5ccccc5Sc5cccc(c52)B4c2cccc4c2N3c2ccccc2S4)cc1]

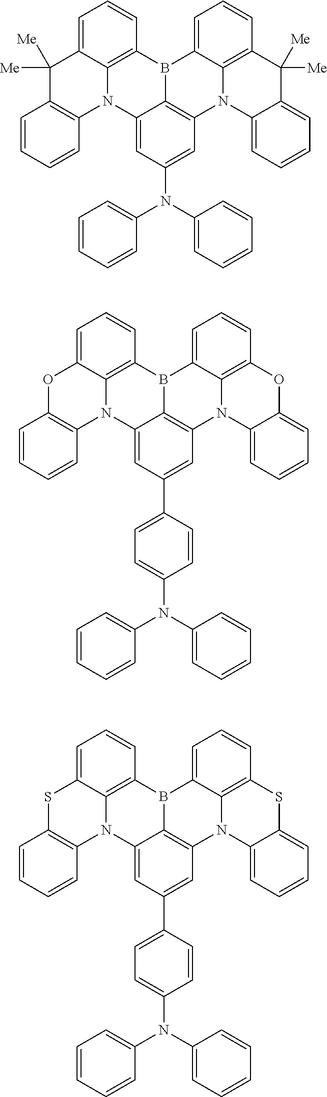

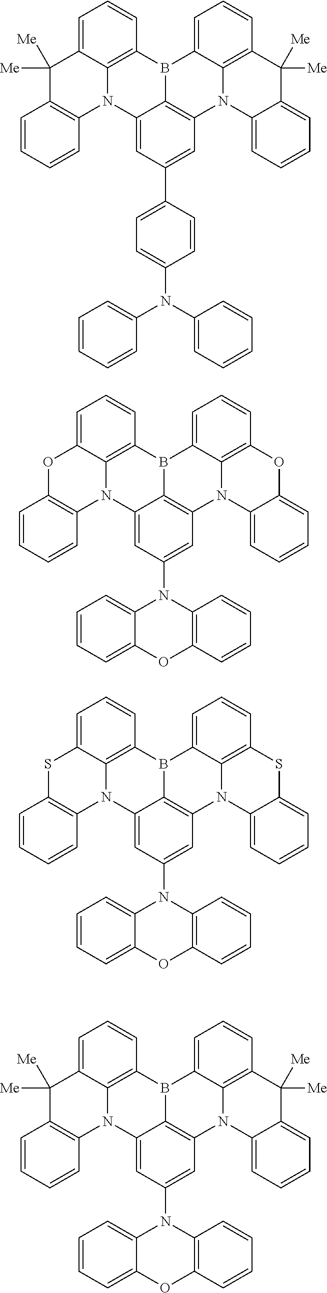

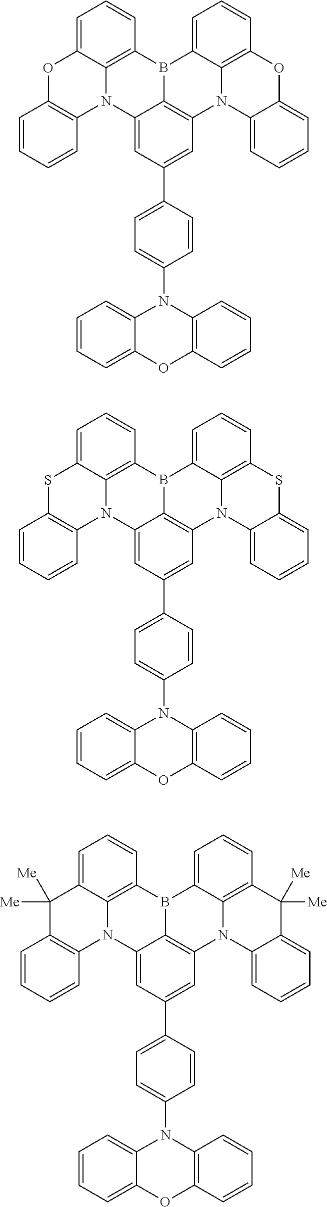

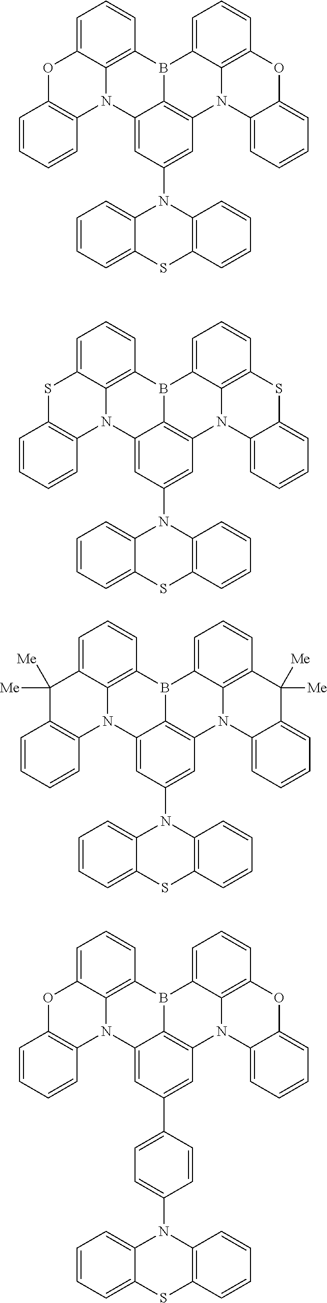

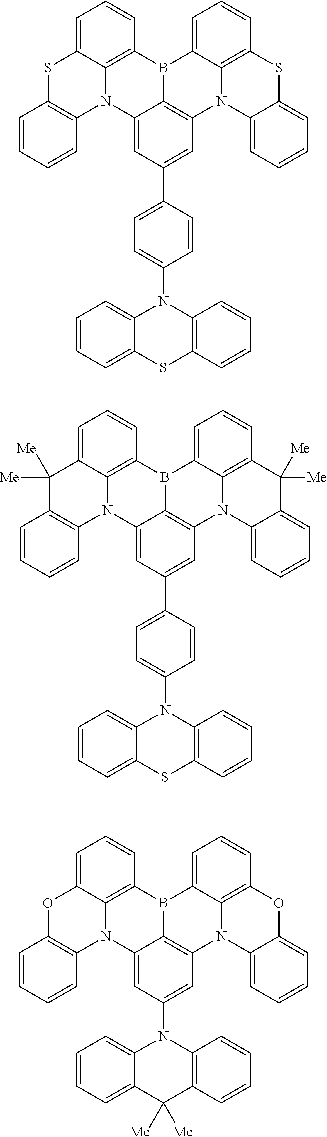

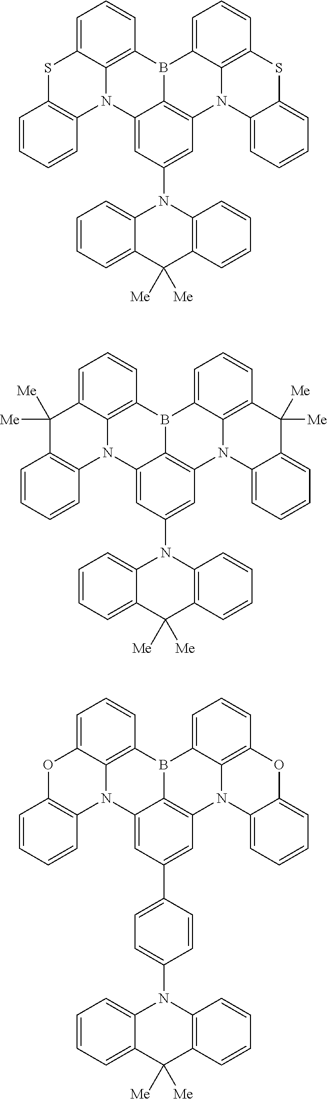 [SMILES: CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

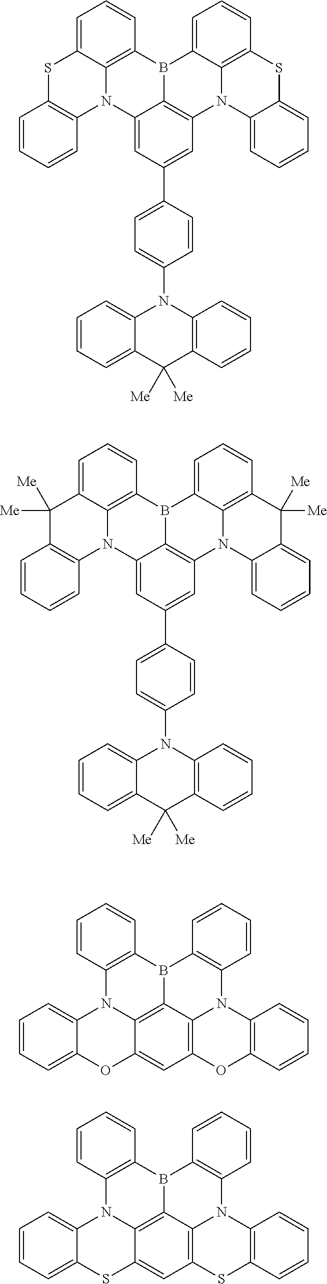

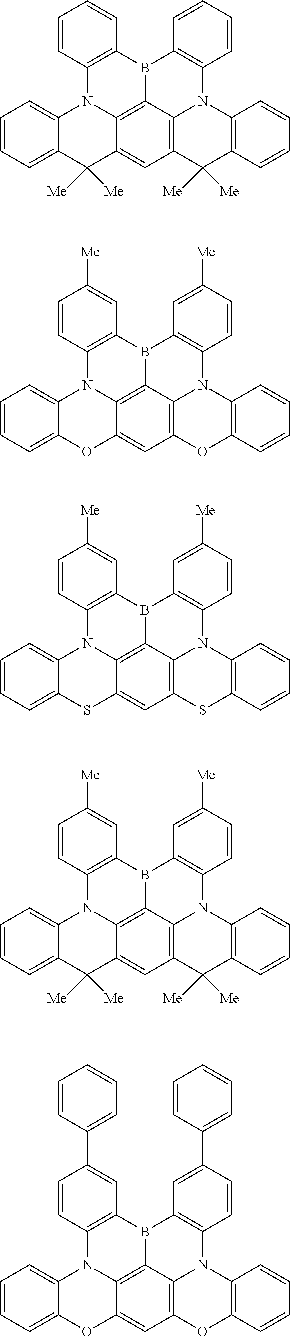 [SMILES: CC1(C)c2ccccc2N2c3ccccc3B3c4ccccc4N4c5ccccc5C(C)(C)c5cc1c2c3c54 ; Cc1ccc2c(c1)B1c3cc(C)ccc3-n3c4ccccc4oc4cc5oc6ccccc6n-2c5c1c43 ; Cc1ccc2c(c1)B1c3cc(C)ccc3-n3c4ccccc4sc4cc5sc6ccccc6n-2c5c1c43 ; Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1]

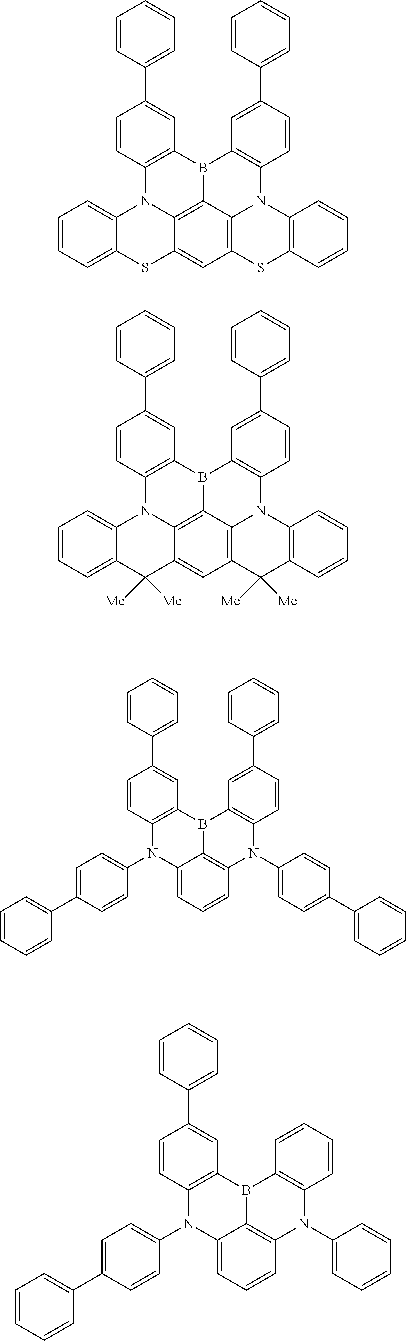

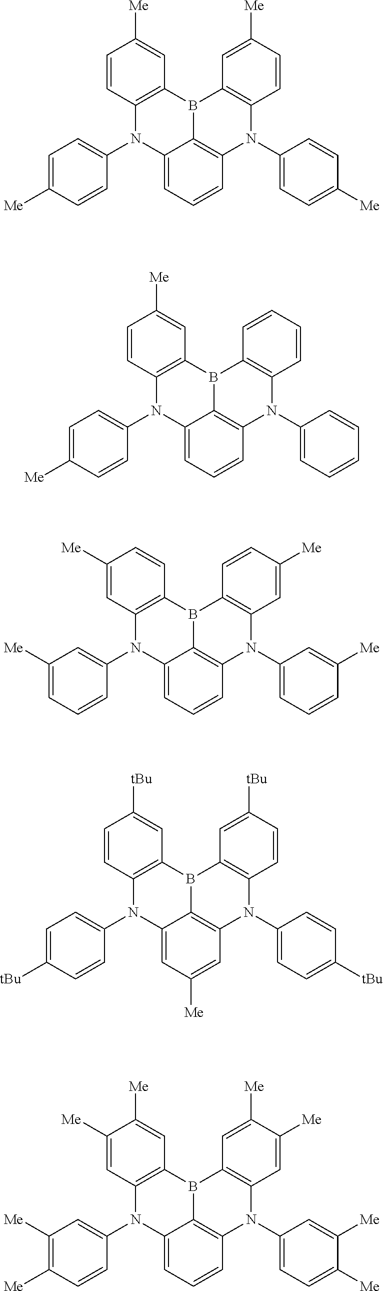 [SMILES: Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; Cc1ccc(N2c3cc(C)c(C)cc3B3c4cc(C)c(C)cc4N(c4ccc(C)c(C)c4)c4cccc2c43)cc1C]

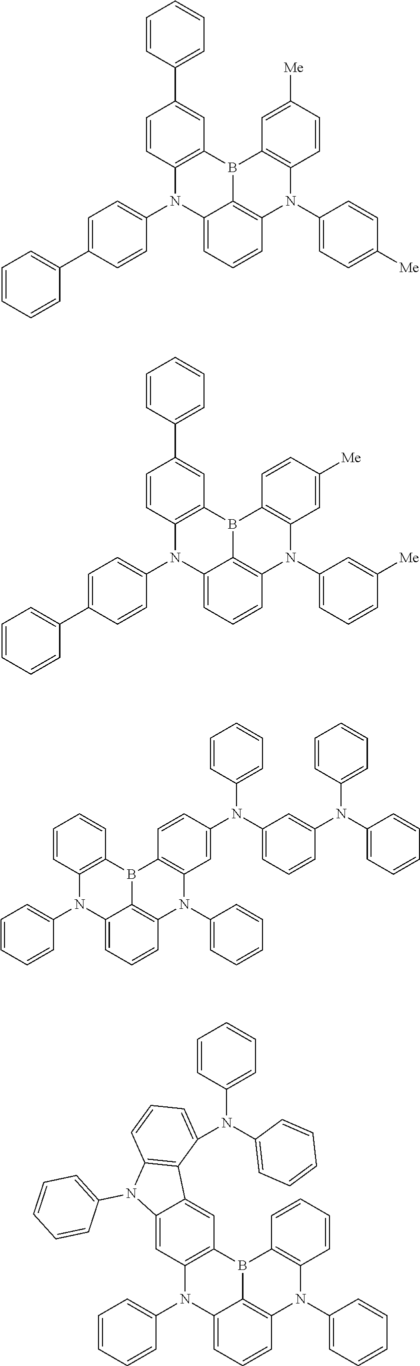 [SMILES: Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1]

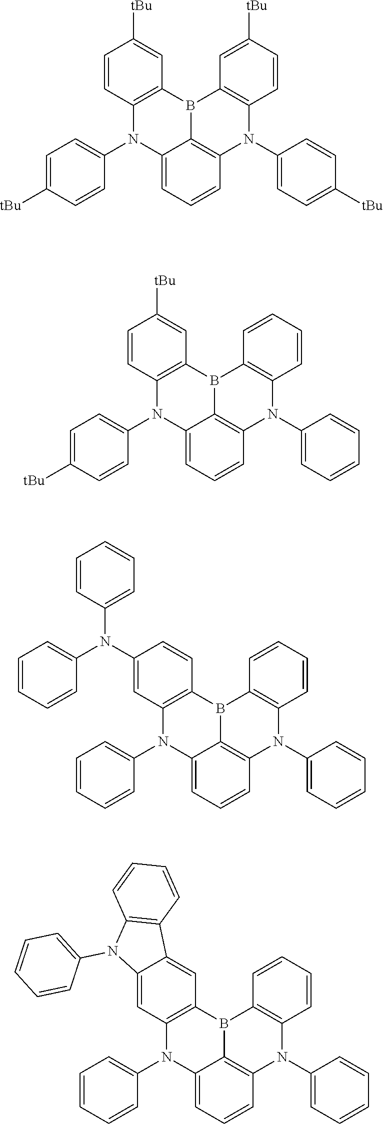 [SMILES: CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1 ; c1ccc(N2c3ccccc3B3c4cc5c6ccccc6n(-c6ccccc6)c5cc4N(c4ccccc4)c4cccc2c43)cc1]

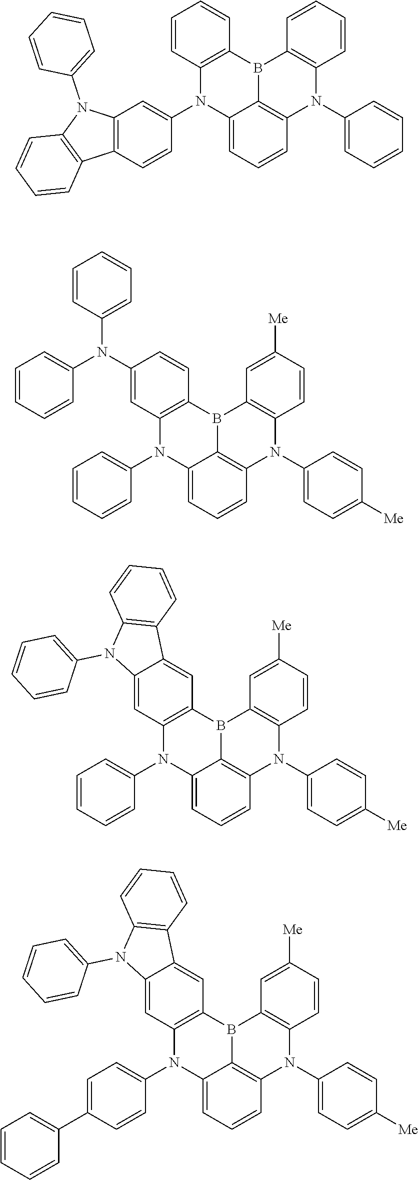 [SMILES: c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

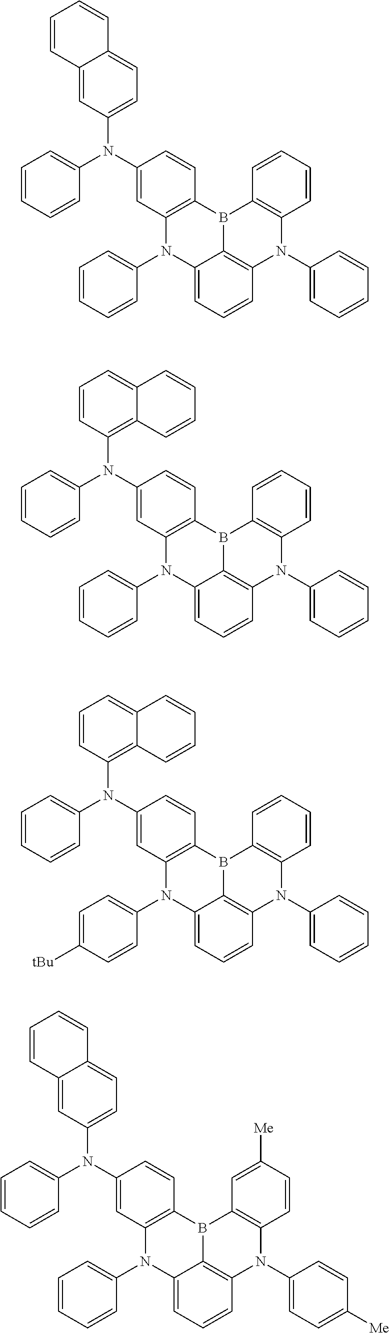 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1 ; Cc1ccc(N2c3ccc(C)cc3B3c4ccc(N(c5ccccc5)c5ccc6ccccc6c5)cc4N(c4ccccc4)c4cccc2c43)cc1]

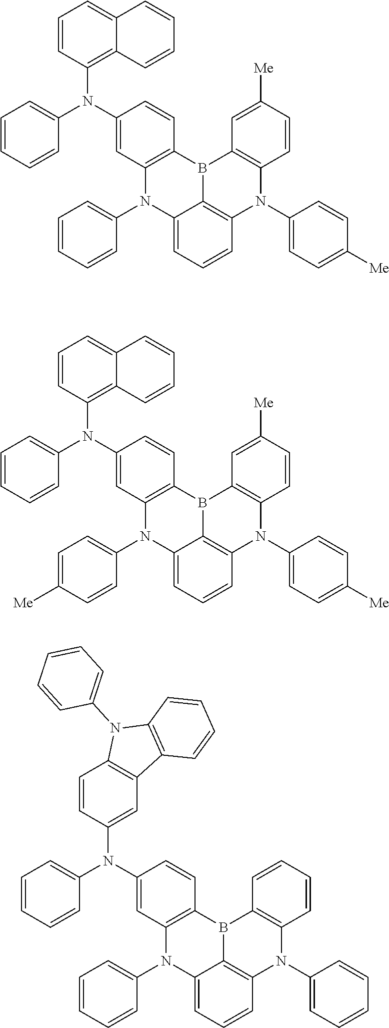

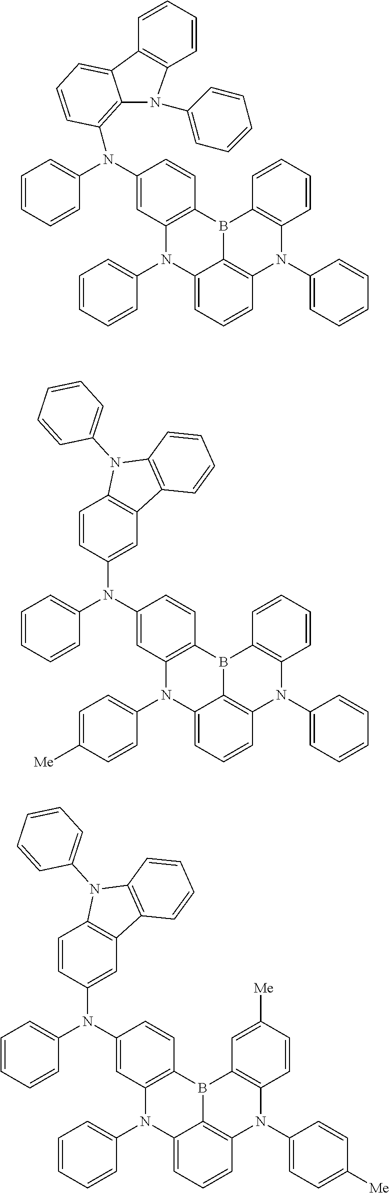

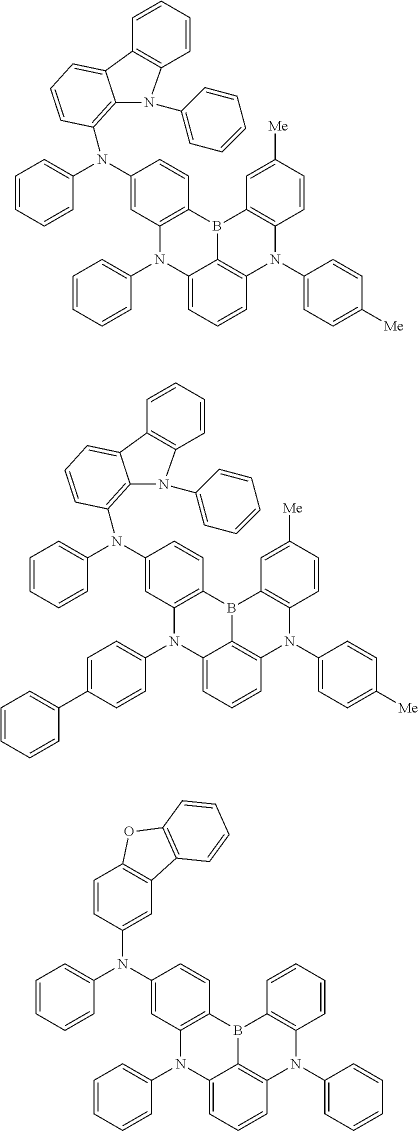 [SMILES: Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1 ; c1ccc(N(c2ccc3c(c2)N(c2ccccc2)c2cccc4c2B3c2ccccc2N4c2ccccc2)c2ccc3oc4ccccc4c3c2)cc1]

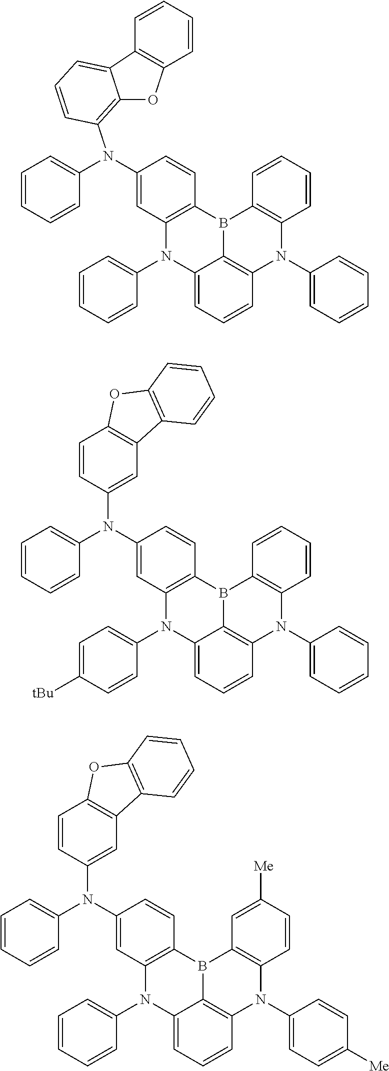 [SMILES: c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccccc4)c4cccc2c43)cc1]

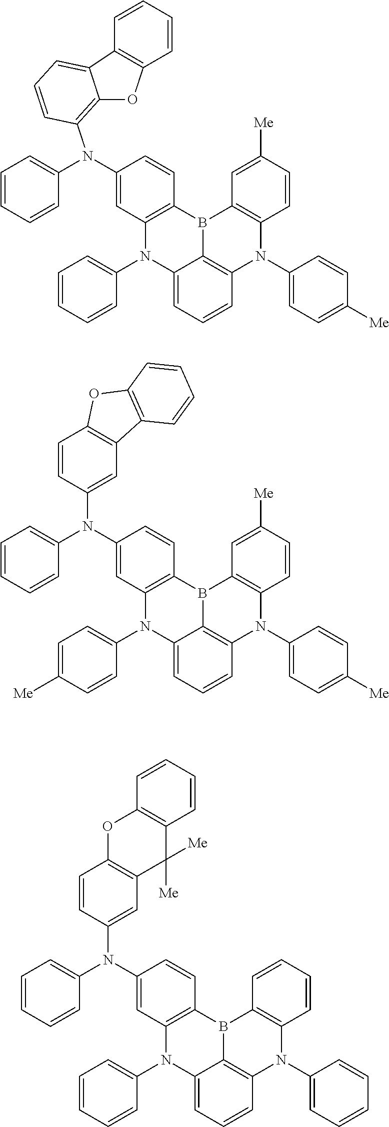 [SMILES: Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1 ; CC1(C)c2ccccc2Oc2ccc(N(c3ccccc3)c3ccc4c(c3)N(c3ccccc3)c3cccc5c3B4c3ccccc3N5c3ccccc3)cc21]

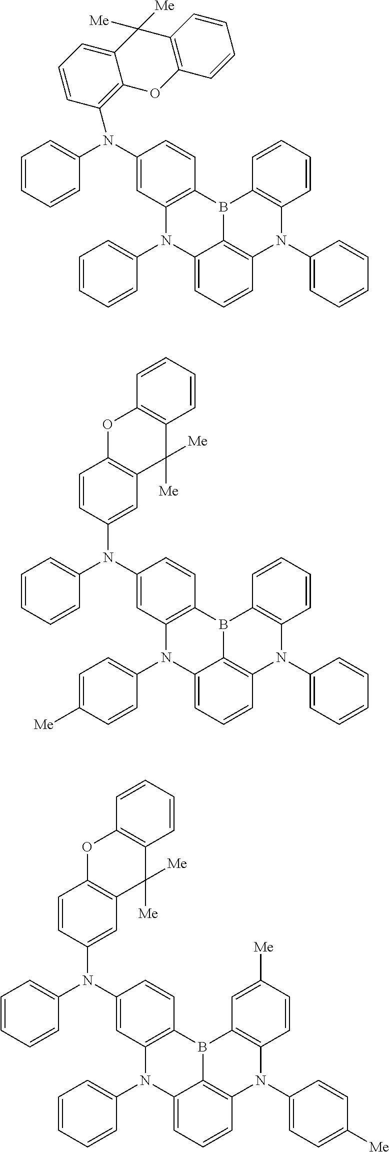 [SMILES: CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1]

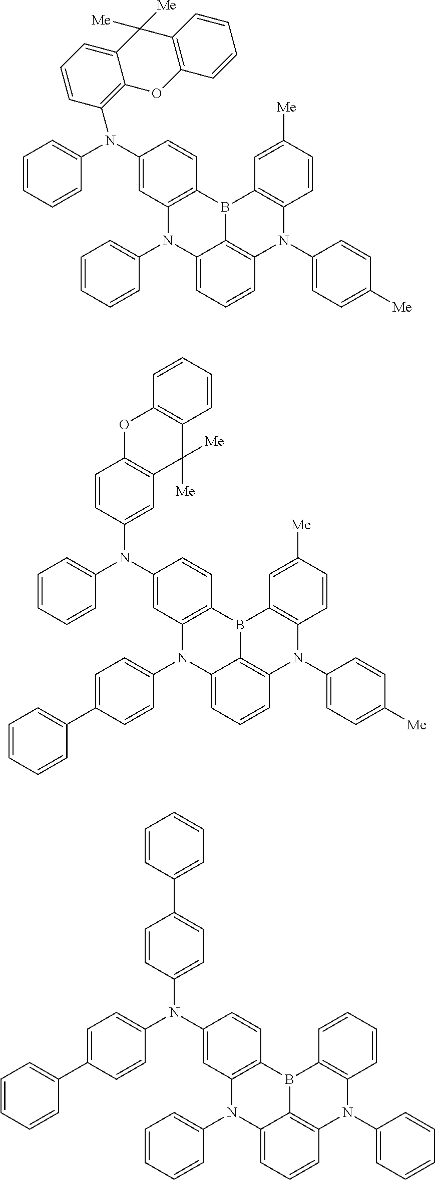 [SMILES: Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1 ; c1ccc(-c2ccc(N(c3ccc(-c4ccccc4)cc3)c3ccc4c(c3)N(c3ccccc3)c3cccc5c3B4c3ccccc3N5c3ccccc3)cc2)cc1]

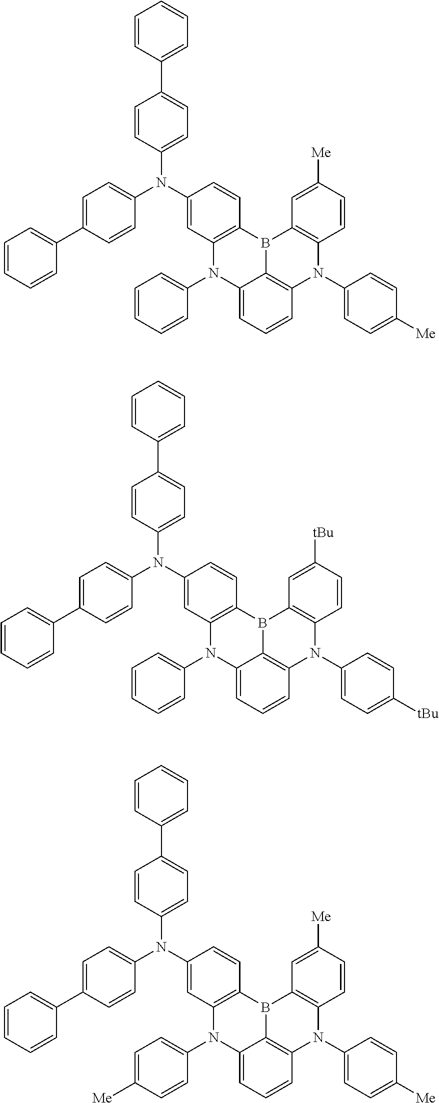 [SMILES: Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1]

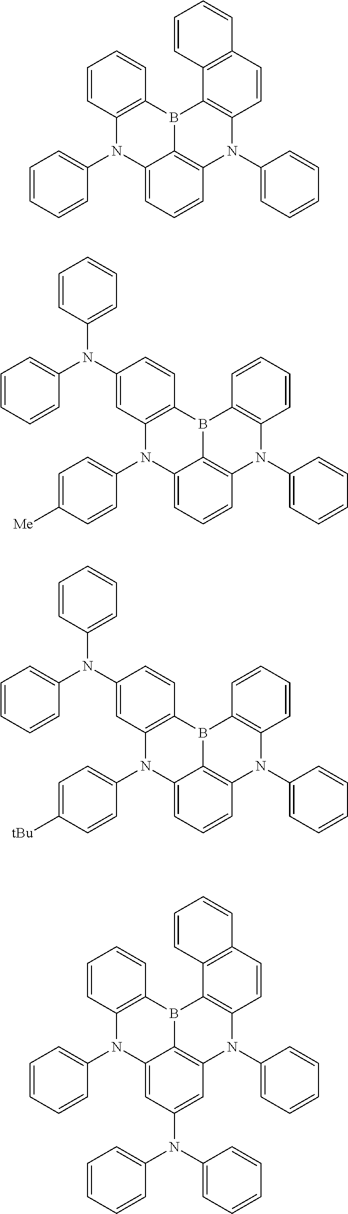

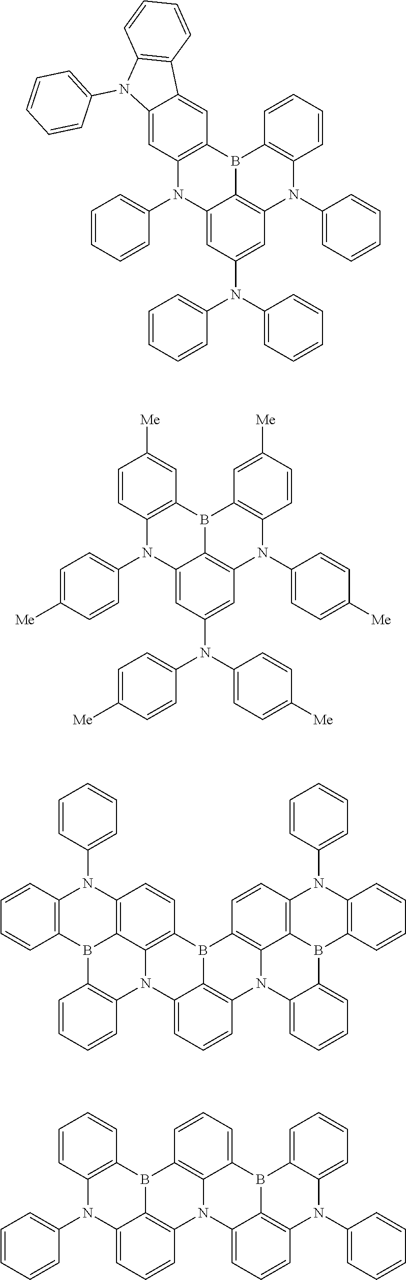 [SMILES: c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1 ; c1ccc(N2c3ccccc3B3c4ccccc4N4c5cccc6c5B(c5ccc2c3c54)c2ccc3c4c2N6c2ccccc2B4c2ccccc2N3c2ccccc2)cc1 ; c1ccc(N2c3ccccc3B3c4cccc5c4N(c4cccc2c43)c2cccc3c2B5c2ccccc2N3c2ccccc2)cc1]

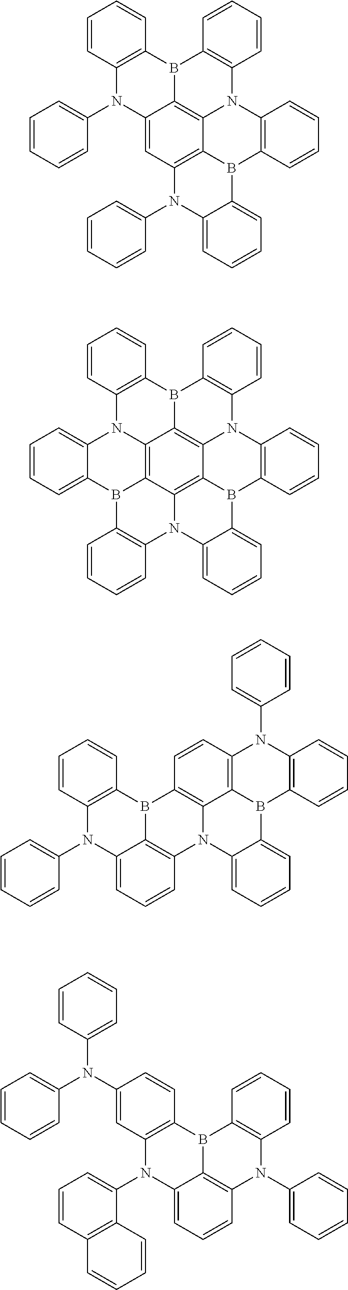 [SMILES: c1ccc(N2c3ccccc3B3c4ccccc4N4c5ccccc5B5c6ccccc6N(c6ccccc6)c6cc2c3c4c65)cc1 ; c1ccc2c(c1)B1c3ccccc3N3c4ccccc4B4c5ccccc5N5c6ccccc6B6c7ccccc7N2c2c1c3c4c5c26 ; c1ccc(N2c3ccccc3B3c4ccc5c6c4N(c4ccccc4B6c4ccccc4N5c4ccccc4)c4cccc2c43)cc1 ; c1ccc(N(c2ccccc2)c2ccc3c(c2)N(c2cccc4ccccc24)c2cccc4c2B3c2ccccc2N4c2ccccc2)cc1]

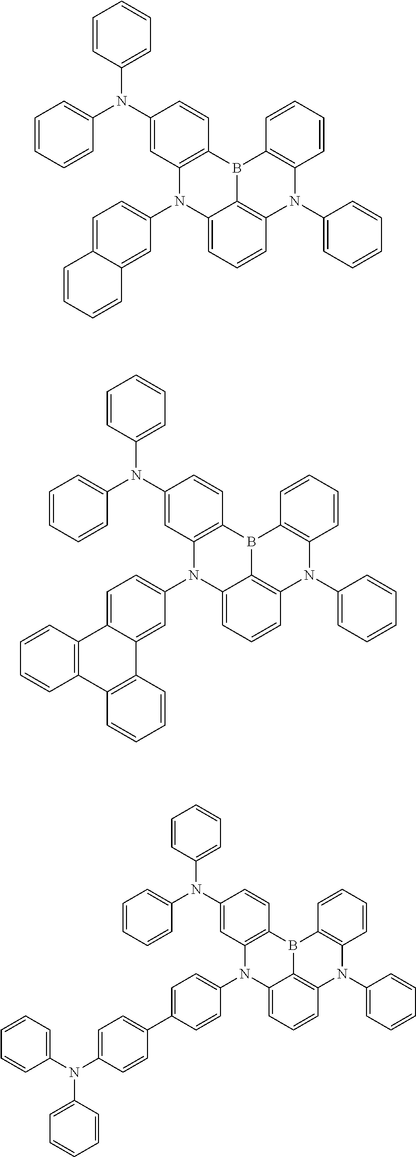 [SMILES: c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1 ; c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1]

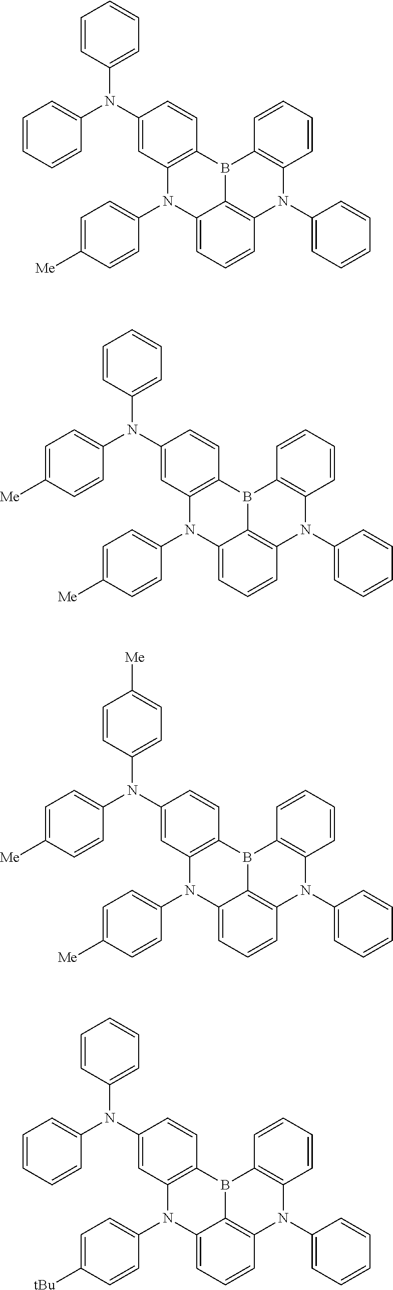 [SMILES: Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

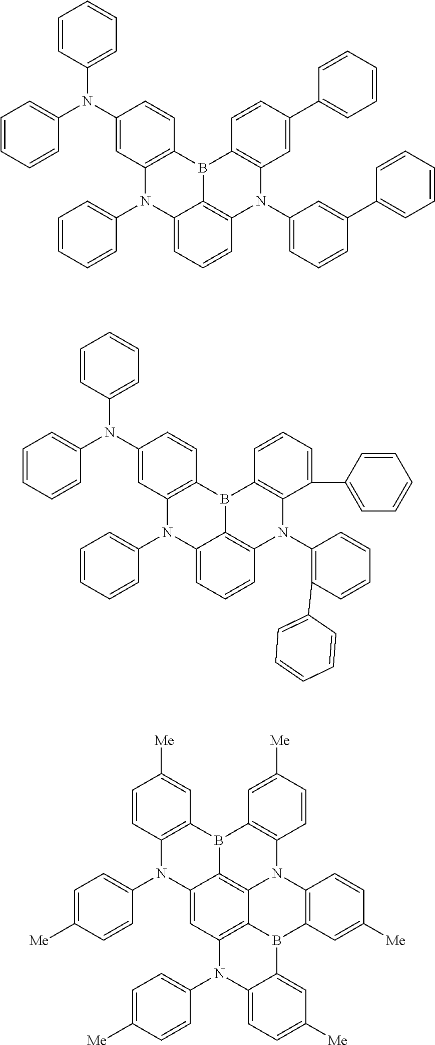 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1]

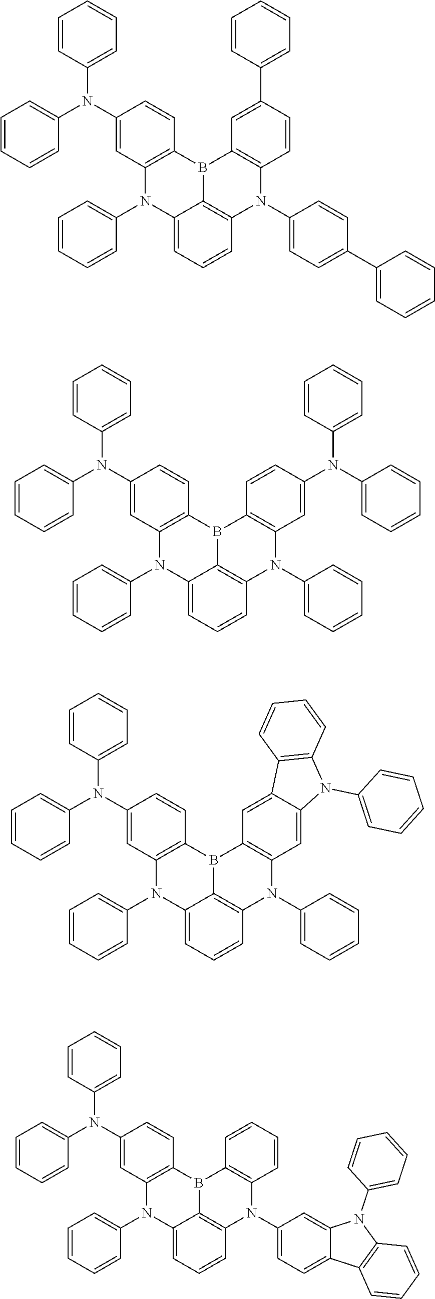 [SMILES: c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1 ; c1ccc(N(c2ccccc2)c2ccc3c(c2)N(c2ccccc2)c2cccc4c2B3c2cc3c5ccccc5n(-c5ccccc5)c3cc2N4c2ccccc2)cc1 ; c1ccc(N(c2ccccc2)c2ccc3c(c2)N(c2ccccc2)c2cccc4c2B3c2ccccc2N4c2ccc3c4ccccc4n(-c4ccccc4)c3c2)cc1]

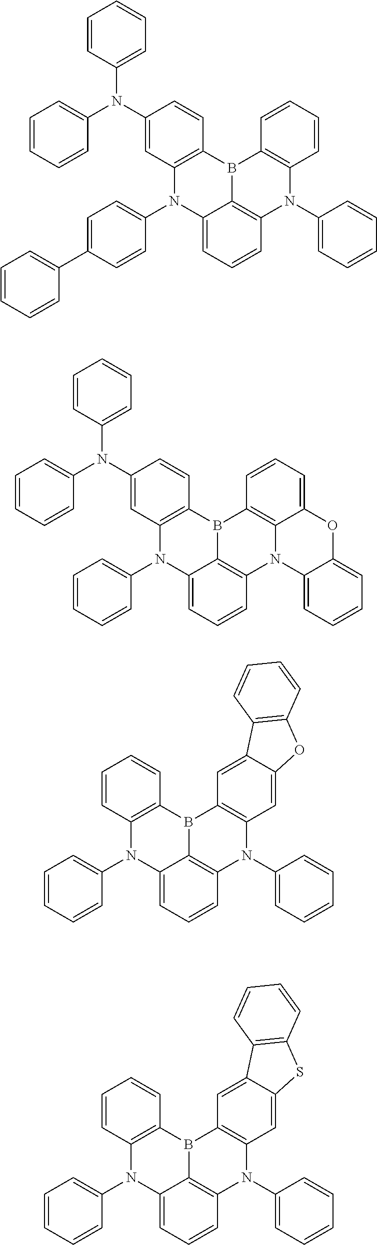 [SMILES: c1ccc(-c2ccc(N3c4cc(N(c5ccccc5)c5ccccc5)ccc4B4c5ccccc5N(c5ccccc5)c5cccc3c54)cc2)cc1 ; c1ccc(N(c2ccccc2)c2ccc3c(c2)N(c2ccccc2)c2cccc4c2B3c2cccc3c2N4c2ccccc2O3)cc1 ; c1ccc(N2c3ccccc3B3c4cc5c(cc4N(c4ccccc4)c4cccc2c43)oc2ccccc25)cc1 ; c1ccc(N2c3ccccc3B3c4cc5c(cc4N(c4ccccc4)c4cccc2c43)sc2ccccc25)cc1]

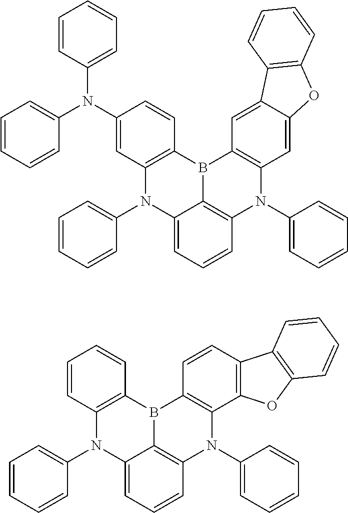 [SMILES: c1ccc(N(c2ccccc2)c2ccc3c(c2)N(c2ccccc2)c2cccc4c2B3c2cc3c(cc2N4c2ccccc2)oc2ccccc23)cc1 ; c1ccc(N2c3ccccc3B3c4ccc5c(oc6ccccc65)c4N(c4ccccc4)c4cccc2c43)cc1]

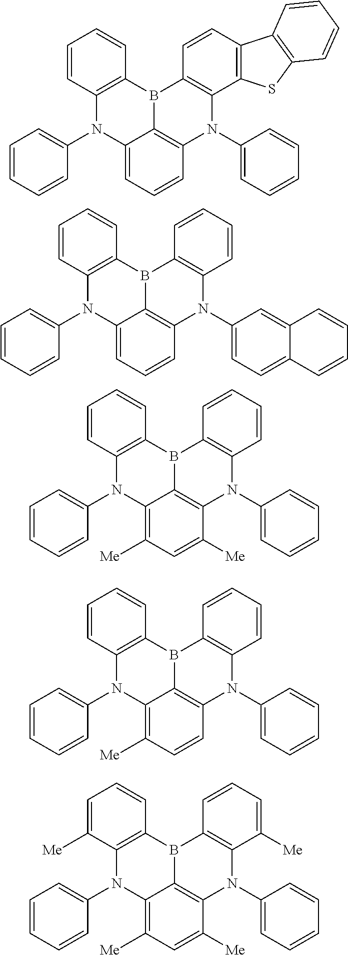

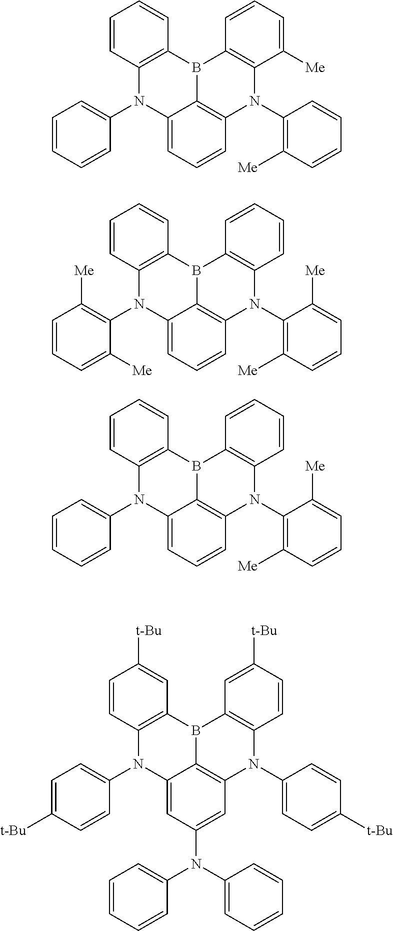

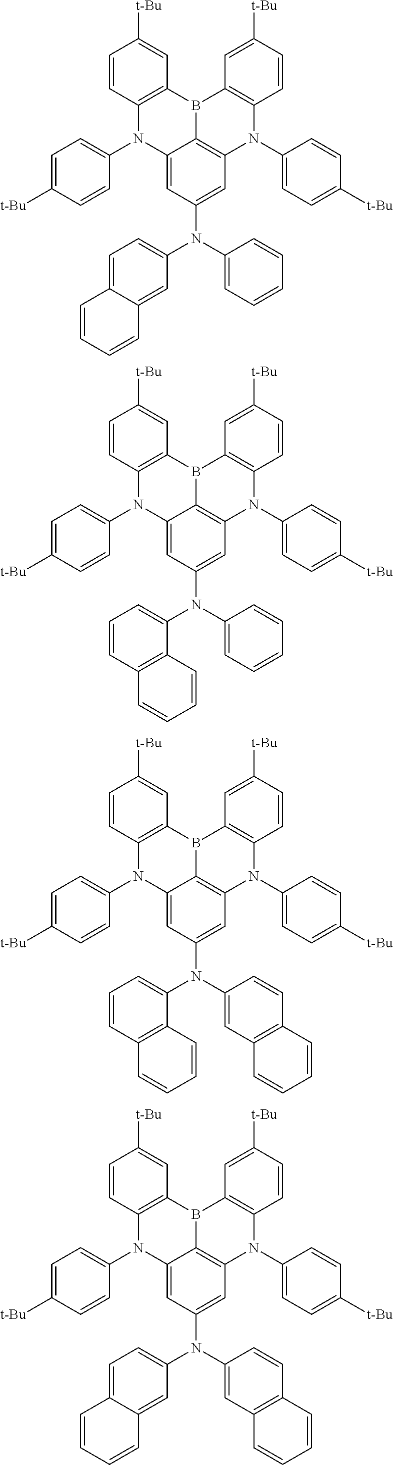

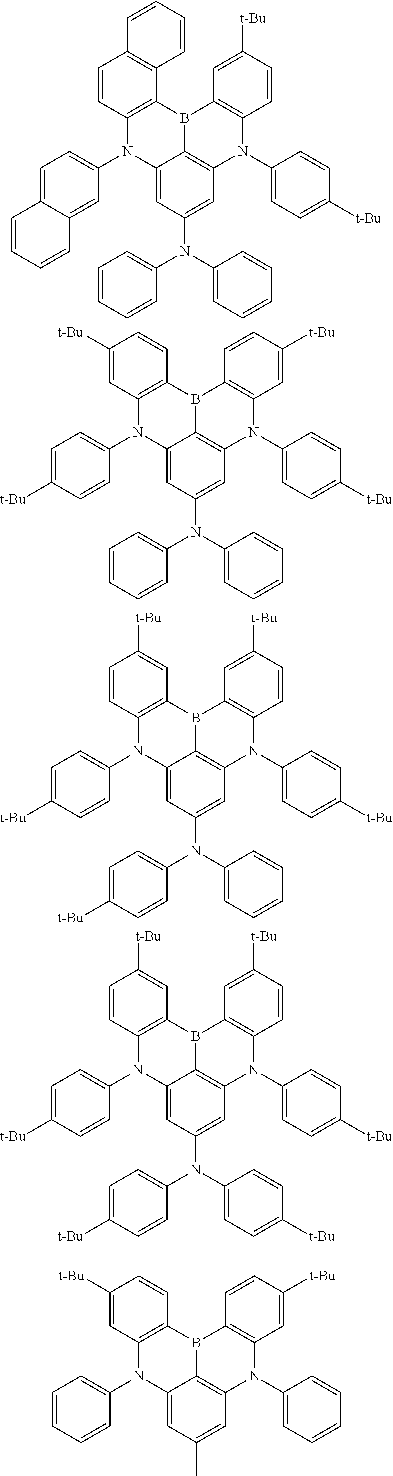

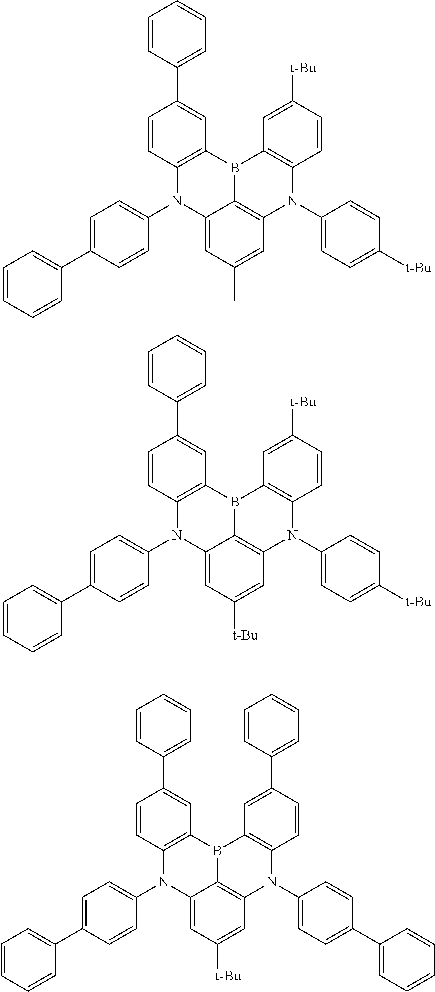 [SMILES: Cc1cc2c3c(c1)N(c1ccc(C(C)(C)C)cc1)c1ccc(C(C)(C)C)cc1B3c1cc(-c3ccccc3)ccc1N2c1ccc(-c2ccccc2)cc1 ; CC(C)(C)c1ccc(N2c3ccc(C(C)(C)C)cc3B3c4cc(-c5ccccc5)ccc4N(c4ccc(-c5ccccc5)cc4)c4cc(C(C)(C)C)cc2c43)cc1 ; CC(C)(C)c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1]

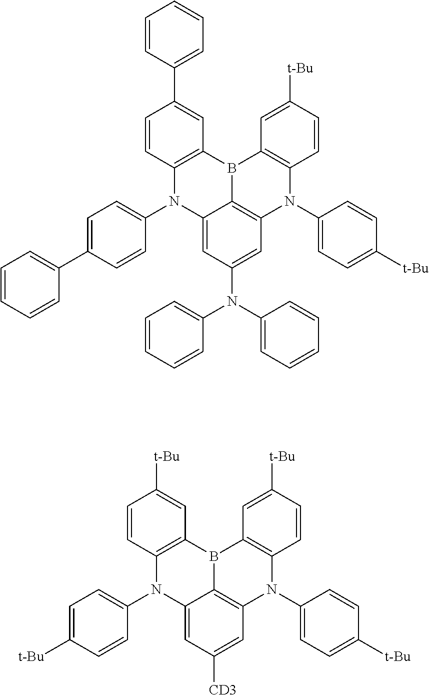 [SMILES: CC(C)(C)c1ccc(N2c3ccc(C(C)(C)C)cc3B3c4cc(-c5ccccc5)ccc4N(c4ccc(-c5ccccc5)cc4)c4cc(N(c5ccccc5)c5ccccc5)cc2c43)cc1 ; [2H]C([2H])([2H])c1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

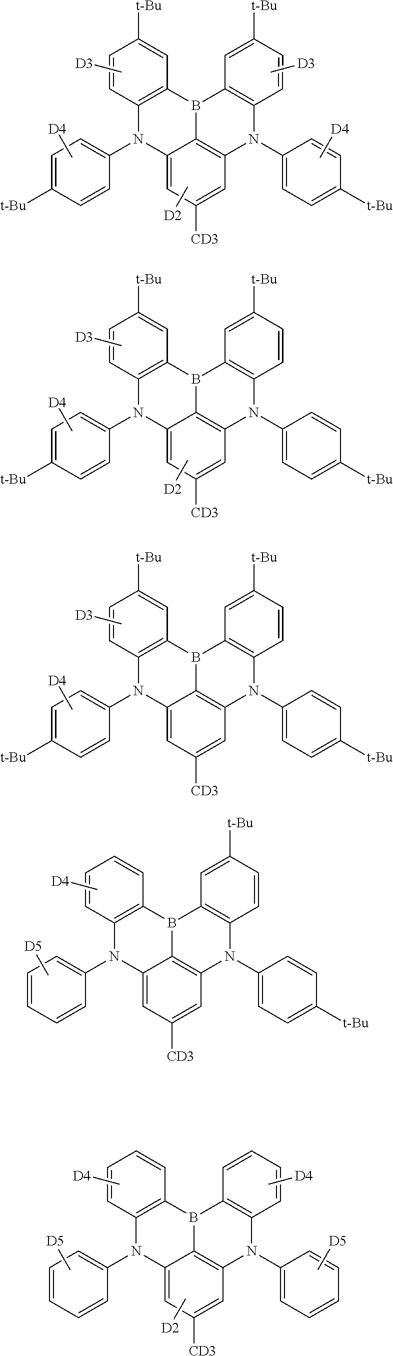

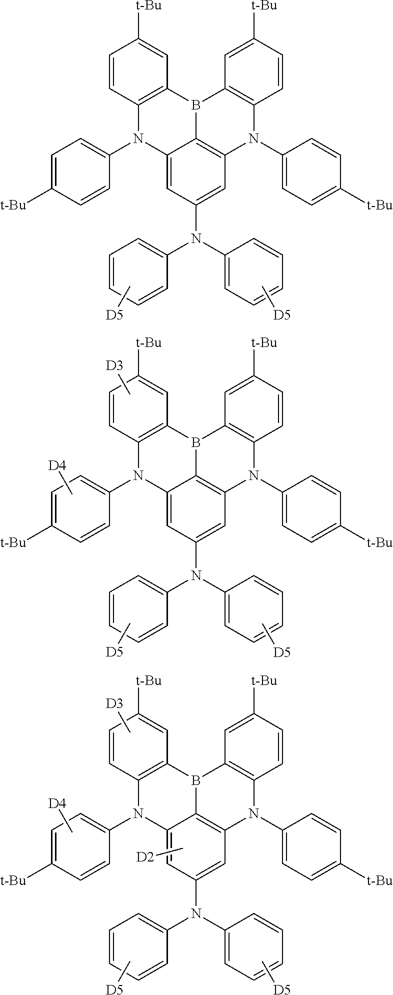

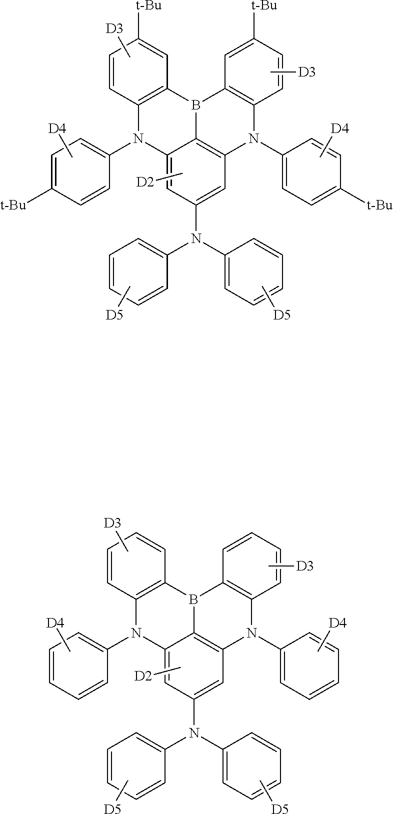

In the above compounds, D2 to D5 each signify that 2 to 5 hydrogens are replaced with deuterium.

The formation of each layer of the organic electroluminescent device of the present disclosure can be accomplished by applying any one of dry film-forming methods such as vacuum deposition, sputtering, plasma, ion plating methods, etc., or wet film-forming methods such as spin coating, dip coating, flow coating methods, etc. When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

According to one embodiment of the present disclosure, when forming a layer of the first host material and the second host material, the layer can be formed by the methods listed above, and often can be formed by a co-deposition or mixed deposition process. Co-deposition is a method for mixing two or more materials into each individual crucible source and applying current to both cells simultaneously to evaporate the materials. Mixed deposition is a method for mixing two or more materials in one crucible source before deposition and then applying current to one cell to evaporate the materials.

According to one embodiment of the present disclosure, when the first host material and the second host material are present in the same layer or different layers in the organic electroluminescent device, the two host compounds may be individually formed into films. For example, the second host material may be deposited after depositing the first host material.

According to one embodiment of the present disclosure, a display system comprising the plurality of host materials comprising the first host compound represented by Formula 1 and the second host compound represented by Formula 2; or the organic electroluminescent compound represented by Formula 11 can be provided. In addition, it is possible to produce a display system, e.g., a display system for smart phones, tablets, notebooks, PCs, TVs, or cars; or a lighting system, e.g., an outdoor or indoor lighting system, by using the organic electroluminescent device of the present disclosure.

Hereinafter, the preparation method of the compound according to the present disclosure and the properties thereof, as well as the driving voltage, conversion efficiency, and lifetime properties of the OLED according to the present disclosure will be explained. However, the following examples are only provided to explain the characteristics of the compound and OLED according to the present disclosure for a detailed understanding of the present disclosure, and the present disclosure is not limited to the following examples.

### Example 1: Preparation of Compound H1-72-D8

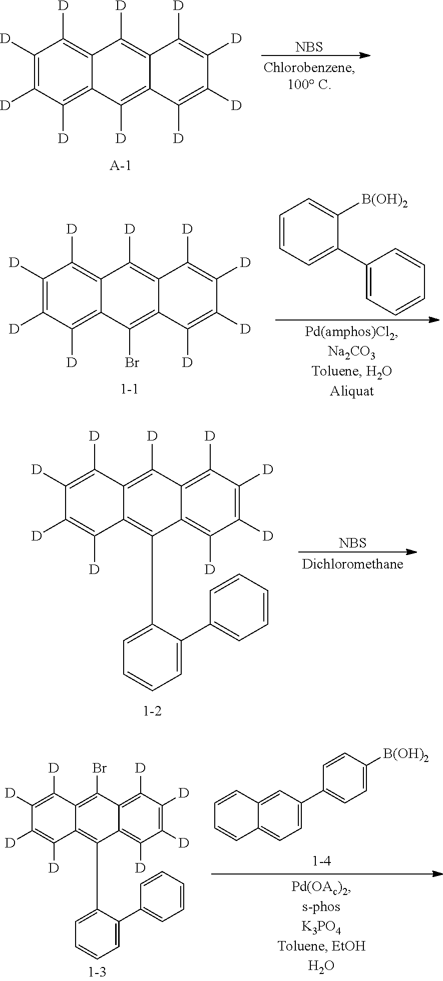

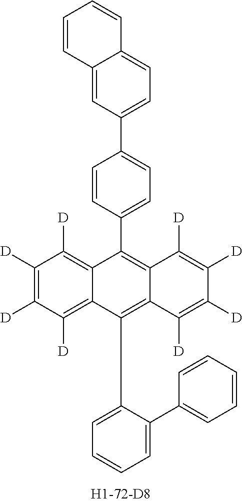 [SMILES: [2H]c1c([2H])c([2H])c2c(-c3ccccc3-c3ccccc3)c3c([2H])c([2H])c([2H])c([2H])c3c(-c3ccc(-c4ccc5ccccc5c4)cc3)c2c1[2H]]

### Synthesis of Compound 1-1

In a flask, Compound A-1 (47 g, 250 mmol) was dissolved in chlorobenzene (1.3 L), and N-bromosuccinimide (NBS) (49 g, 275 mmol) was added thereto, and then the mixture was reacted at 100° C. for 7 hours. After completion of the reaction, the mixture was neutralized with K₂CO₃ and sodium thiosulfate aqueous solution, the organic layer was extracted with dichloromethane, and treated with MgSO₄. The organic layer was filtered and concentrated, and the resulting mixture was purified by column chromatography to obtain Compound 1-1 (49.6 g, yield: 74%).

### Synthesis of Compound 1-2

In a 3 L of round bottom flask (RBF), Compound 1-1 (49.6 g, 186 mmol), [1,1′-biphenyl]-2-ylboronic acid (44.3 g, 224 mmol), bis(di-tert-butyl(4-dimethylamino-phenyl)phosphine)dichloropalladium (II) (13.1 g, 18.6 mmol), Aliquat (7.5 g, 18.6 mmol), sodium carbonate (39.4 g, 372 mmol), 1.2 L of toluene, and 372 mL of distilled water were added, and the mixture was stirred under reflux at 140° C. for 4 hours. After completion of the reaction, the mixture was cooled to room temperature, and an organic layer was extracted with dichloromethane, and treated with MgSO₄. The organic layer was filtered and concentrated, and the resulting mixture was purified by column chromatography to obtain Compound 1-2 (54 g, yield: 85%).

### Synthesis of Compound 1-3

In a flask, Compound 1-2 (54 g, 159 mmol) was dissolved in dichloromethane (1.1 L), and N-bromosuccinimide (42.5 g, 239 mmol) was added thereto, and then the mixture was reacted at 35° C. for 20 hours. After completion of the reaction, the mixture was neutralized with K₂CO₃ and sodium thiosulfate aqueous solution, the organic layer was extracted with dichloromethane and treated with MgSO₄. The organic layer was filtered and concentrated, and the resulting mixture was purified by column chromatography to obtain Compound 1-3 (50 g, yield: 77%).

### Synthesis of Compound H1-72-D8

In a flask, Compound 1-3 (12.4 g, 30 mmol), Compound 1-4 (10 g, 30 mmol), Pd(OAc)₂ (337 mg, 1.5 mmol), SPhos (1.2 g, 3 mmol), K₃PO₄ (16 g, 75 mmol), toluene (150 mL), ethanol (40 mL), and distilled water (40 mL) were added, and the mixture was stirred under reflux for 3 hours. The mixture was cooled to room temperature, and an organic layer was extracted with ethyl acetate, and dried with magnesium sulfate. The organic layer was distilled under reduced pressure and separated by column chromatography to obtain Compound H1-72-D8 (13 g, yield: 80%).

| | MW | M.P. |
|---|---|---|
| H1-72-D8 | 540.69 | 248° C. |

### Example 2: Preparation of Compound H1-43

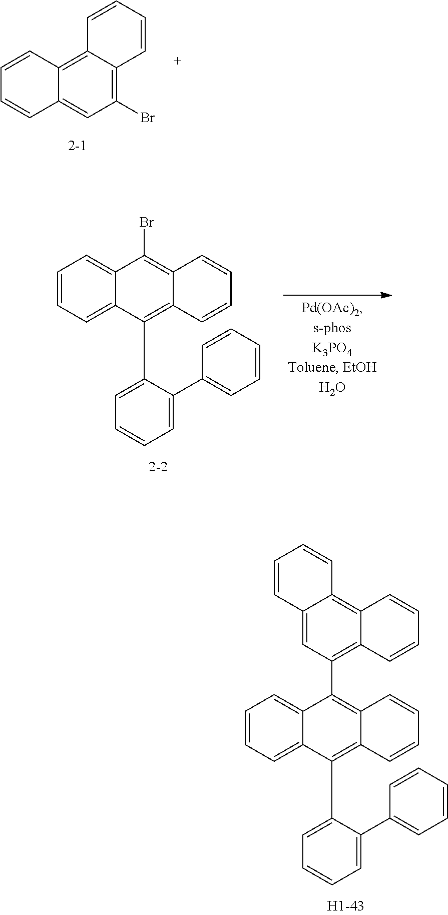

In a flask, Compound 2-1 (11 g, 42 mmol), Compound 2-2 (15 g, 40 mmol), Pd(OAc)₂ (449 mg, 2.0 mmol), SPhos (1.7 g, 4.0 mmol), K₃PO₄ (22 g, 105 mmol), toluene (200 mL), ethanol (40 mL), and distilled water (40 mL) were added, and the mixture was stirred under reflux for 3 hours. The mixture was cooled to room temperature, and an organic layer was extracted with ethyl acetate, and dried with magnesium sulfate. The organic layer was distilled under reduced pressure and separated by column chromatography to obtain Compound H1-43 (6.4 g, yield: 32%).

| | MW | M.P. |
|---|---|---|
| H1-43 | 506.65 | 244° C. |

### Example 3: Preparation of Compound H1-2

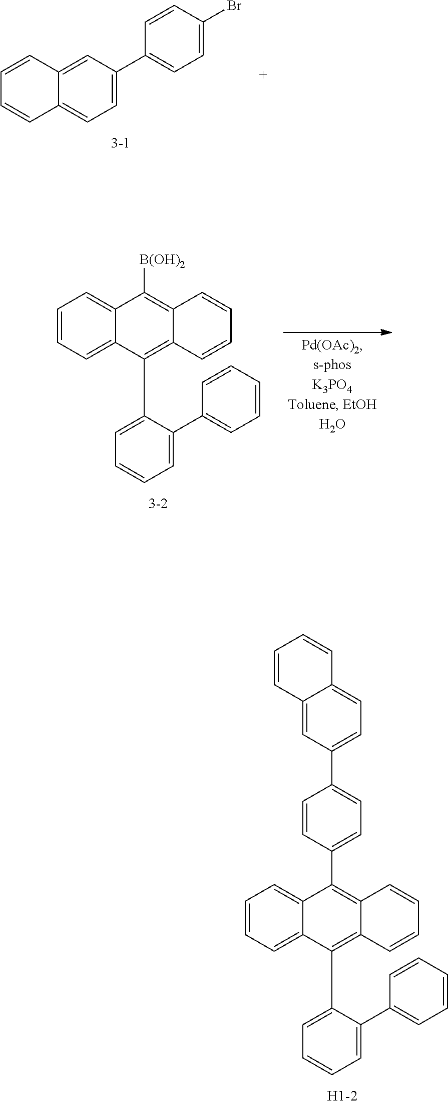

In a flask, Compound 3-1 (20 g, 71 mmol), Compound 3-2 (24 g, 64 mmol), Pd(OAc)₂ (797 mg, 3.55 mmol), SPhos (2.9 g, 7.1 mmol), K₃PO₄ (38 g, 178 mmol), toluene (360 mL), ethanol (90 mL), and distilled water (90 mL) were added, and the mixture was stirred under reflux for 5 hours. The mixture was cooled to room temperature, and an organic layer was extracted with ethyl acetate (EA), and dried with magnesium sulfate. The organic layer was distilled under reduced pressure and separated by column chromatography to obtain Compound H1-2 (24 g, yield: 70%).

| | MW |
|---|---|
| H1-2 | 532.69 |

### Example 4: Preparation of Compound H1-40

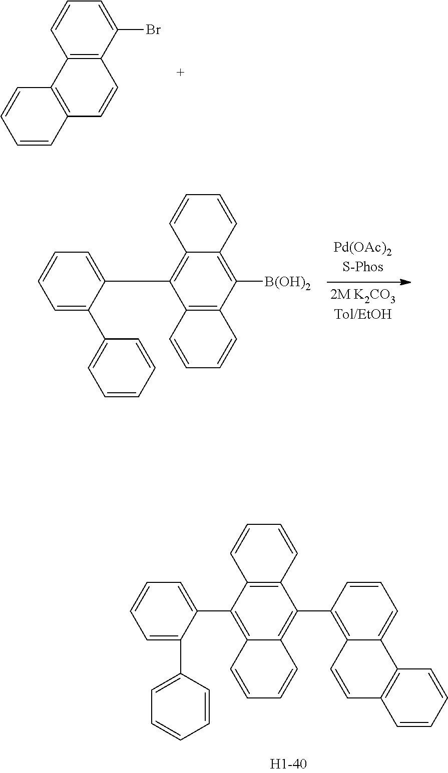

In a flask, 1-bromophenanthrene (15 g, 58.33 mmol), (10-([1,1′-biphenyl]-2-yl)anthracen-9-yl)boronic acid (24.01 g, 64.17 mmol), Pd(OAc)₂ (0.65 g, 2.91 mmol), SPhos (2.39 g, 5.83 mmol), K₂CO₃ (20.15 g, 145.8 mmol), 400 mL of toluene, 100 mL of distilled water, and 50 mL of ethanol were added, and the mixture was stirred under reflux. After 2 hours, the mixture was cooled to room temperature. Distilled water was added, and the organic layer was extracted with EA. Magnesium sulfate was added to the organic layer, dried, and filtered under reduced pressure. The organic layer was distilled under reduced pressure and separated by column chromatography to obtain Compound H1-40 (14.0 g, yield: 47.37%).

| | MW | M.P. |
|---|---|---|
| H1-40 | 506.65 | 194.5° C. |

### Example 5: Preparation of Compound H2-256

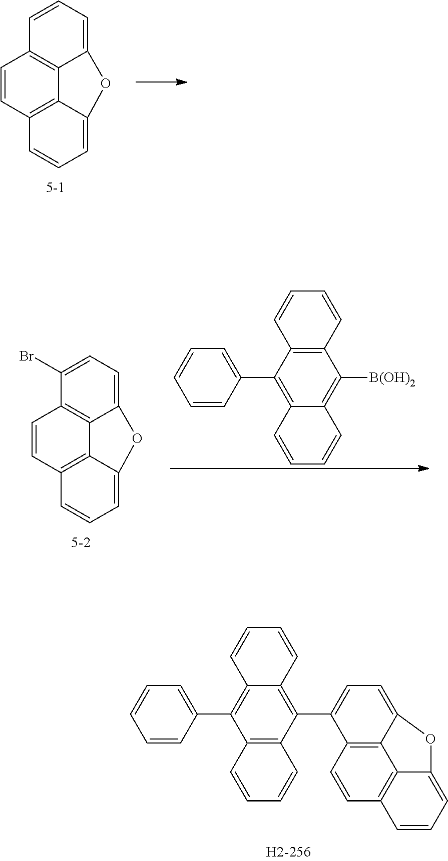 [SMILES: c1cc2ccc3cccc4oc(c1)c2c34 ; Brc1ccc2oc3cccc4ccc1c2c43 ; OB(O)c1c2ccccc2c(-c2ccccc2)c2ccccc12 ; c1ccc(-c2c3ccccc3c(-c3ccc4oc5cccc6ccc3c4c65)c3ccccc23)cc1]

### Synthesis of Compound 5-2

Compound 5-1 (30 g, 156.07 mmol) was dissolved in 900 mL of methylene chloride (MC), and NBS (30.5 g, 171.67 mmol) was added. The mixture was stirred under reflux for 2 hours, cooled to room temperature, and stirred for 15 hours. Aqueous sodium thiosulfate solution was added to the mixture and stirred. The organic layer was separated and neutralized by adding Na₂CO₃ aqueous solution to the organic layer. The organic layer was separated, dried with magnesium sulfate, filtered under reduced pressure, and distilled under reduced pressure. The residue was separated by column chromatography to obtain Compound 5-2 (36 g, yield: 85.10%).

### Synthesis of Compound H2-256

In a flask, Compound 5-2 (20 g, 73.77 mmol), (10-phenylanthracen-9-yl) boronic acid (24.19 g, 81.14 mmol), Pd(OAc)₂ (0.66 g, 2.95 mmol), SPhos (3.63 g, 8.85 mmol), 400 mL of toluene, K₃PO₄ (39.1 g, 184.4 mmol), 90 mL of distilled water, and 90 mL of ethanol were added, and the mixture was stirred under reflux. After 4 hours, the mixture was cooled to room temperature. The organic layer was extracted with EA and washed with distilled water. The organic layer was dried with magnesium sulfate, and filtered under reduced pressure. The organic layer was distilled under reduced pressure and separated by column chromatography to obtain Compound H2-256 (26 g, yield: 79.38%).

| | MW | M.P. |
|---|---|---|
| H2-256 | 444.53 | 270.2° C. |

### Example 6: Preparation of Compound H2-691-D14

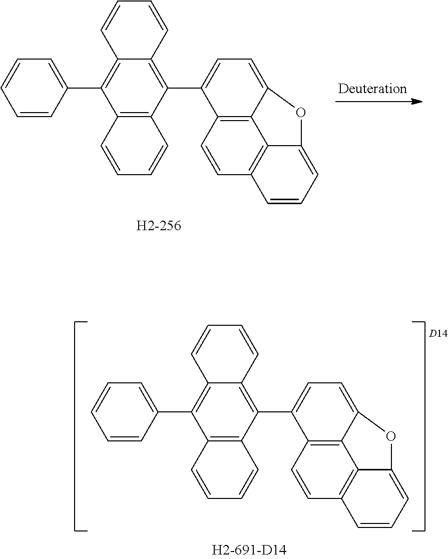

Compound H2-256 was synthesized by the deuteration method disclosed in Korean Patent No. 10-2283849 or 10-1427457, etc. to obtain Compound H2-691-D14 (16.4 g, yield: 72.34%, MS: [M+H]⁺=459.3).

| | MW | M.P. |
|---|---|---|
| H2-691-D14 | 458.3 | 268.9° C. |

### Example 7: Preparation of Compound H2-259

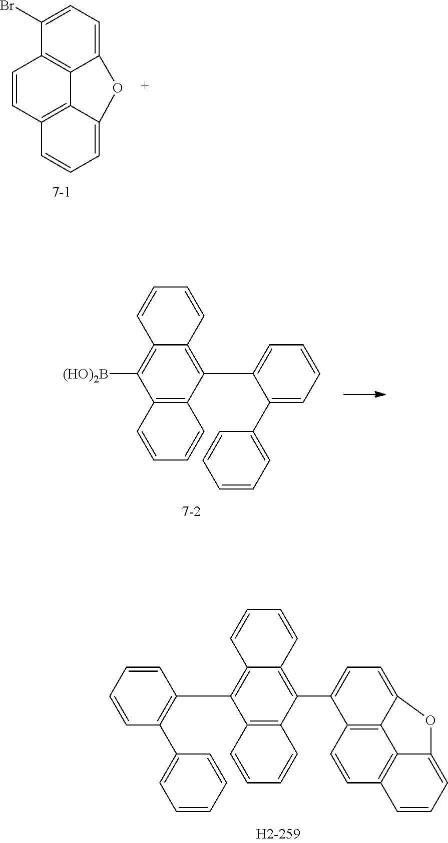 [SMILES: Brc1ccc2oc3cccc4ccc1c2c43 ; OB(O)c1c2ccccc2c(-c2ccccc2-c2ccccc2)c2ccccc12 ; c1ccc(-c2ccccc2-c2c3ccccc3c(-c3ccc4oc5cccc6ccc3c4c65)c3ccccc23)cc1]

In a flask, Compound 7-1 (16 g, 59.01 mmol), Compound 7-2 (24.29 g, 64.91 mmol), Pd(OAc)₂ (0.53 g, 2.36 mmol), SPhos (2.42 g, 5.90 mmol), 400 mL of toluene, K₃PO₄ (25.05 g, 118.03 mmol), 80 mL of distilled water, and 40 mL of ethanol were added, and the mixture was stirred under reflux. After 2 hours, the mixture was cooled to room temperature. The organic layer was extracted with EA and washed with distilled water. The organic layer was dried with magnesium sulfate, and filtered under reduced pressure. The organic layer was distilled under reduced pressure and separated by column chromatography to obtain Compound H2-259 (14.4 g, yield: 46.87%).

| | MW | M.P. |
|---|---|---|
| H2-259 | 520.63 | 250.4° C. |

### Example 8: Preparation of Compound H2-694-D9

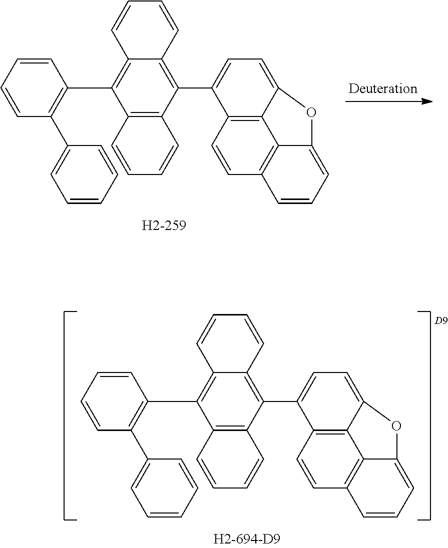

Compound H2-259 was synthesized by the deuteration method disclosed in Korean Patent No. 10-2283849 or 10-1427457, etc. to obtain Compound H2-694-D9 (7.4 g, yield: 60.70%, MS: [M+H]⁺=530.1).

| | MW | M.P. |
|---|---|---|
| H2-694-D9 | 529.1 | 240.1° C. |

### Device Examples 1-1 to 1-6: Producing an OLED Comprising the Compound According to the Present Disclosure as Hosts

An OLED according to the present disclosure was produced. A transparent electrode indium tin oxide (ITO) thin film (10Ω/sq) on a glass substrate for an OLED (GEOMATEC CO., LTD.) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI was introduced into a cell of the vacuum vapor deposition apparatus, and Compound HT-1 was introduced into another cell of the vacuum vapor deposition apparatus. The two materials were evaporated at different rates, and Compound HI was deposited in a doping amount of 5 wt % based on the total amount of Compound HI and Compound HT-1 to form a hole injection layer having a thickness of 10 nm. Compound HT-1 was then deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 15 nm on the first hole transport layer. After forming the hole injection layer and the hole transport layers, a light-emitting layer was formed thereon as follows. The compounds shown in Table 1 below were introduced into two cells of the vacuum vapor deposition apparatus at a ratio of 1:1 as hosts, and Compound BD was introduced into another cell as a dopant. The host materials were evaporated, and the dopant material was simultaneously evaporated at a different rate, and the dopant was deposited in a doping amount of 2 wt % based on the total amount of the hosts and the dopant to form a light-emitting layer having a thickness of 22.5 nm on the second hole transport layer. Compound ET-1 was deposited on the light-emitting layer with a thickness of 5 nm as an electron buffer layer. Compound EI-1 and Compound EI-2 were then added to two other cells and evaporated at a ratio of 2:1 to deposit an electron transport layer having a thickness of 25 nm on the electron buffer layer. In two other cells, Compound Yb (ytterbium) and Compound LiF (lithium fluoride) were added and evaporated at a rate of 2:1 (Yb:LiF) to deposit an electron injection layer with a thickness of 1 nm on the electron transport layer, and an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ Torr.

### Comparative Example 1-1: Producing an OLED Comprising a Comparative Compound as a Host

An OLED was produced in the same manner as in Device Example 1-1, except that only the compound shown in Table 1 below was used as a host of the light-emitting layer.

The driving voltage, conversion efficiency, and the time taken for luminance to decrease from 100% to 95% (lifetime; T₉₅) at a luminance of 1,000 nit of the OLEDs produced in Device Examples 1-1 to 1-6 and Comparative Example 1-1 are provided in Table 1 below. Herein, the conversion efficiency [Eff/Y] means the current efficiency [cd/A] divided by the Y coordinate value of CIE.

TABLE 1

| | | | Driving | Conversion | Lifetime |
|---|---|---|---|---|---|
| | | | Voltage | Efficiency | (T₉₅) |
| | First Host | Second Host | [V] | [Eff/Y] | [hr] |
| Device Example 1-1 | H1-2 — | 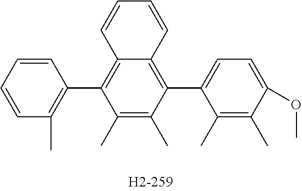 [SMILES: COc1ccc(-c2c(C)c(C)c(-c3ccccc3C)c3ccccc23)c(C)c1C] | 3.7 | 78 | 87 |
| Device Example 1-2 | H1-2 — | 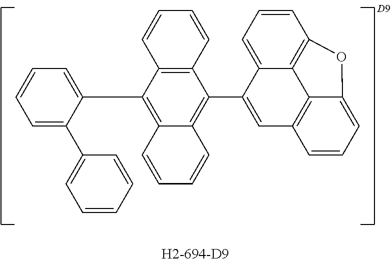 | 3.7 | 77 | 107 |
| Device Example 1-3 | H1-2 — | 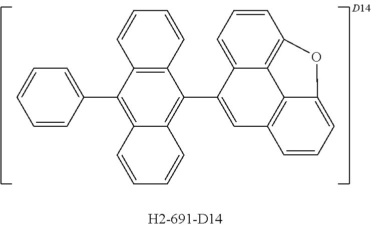 | 3.7 | 82 | 102 |
| Device Example 1-4 | H1-2 — | 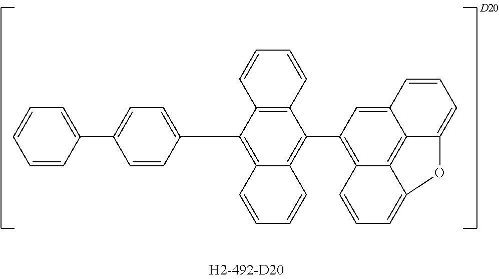 | 3.7 | 87 | 83 |
| Device Example 1-5 | H1-2 — | 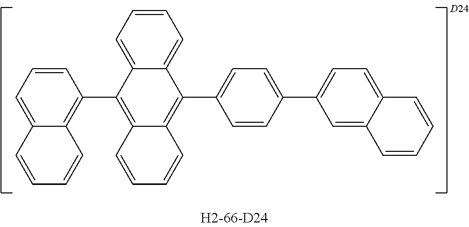 | 3.9 | 90 | 114 |
| Device Example 1-6 | H1-2 — | 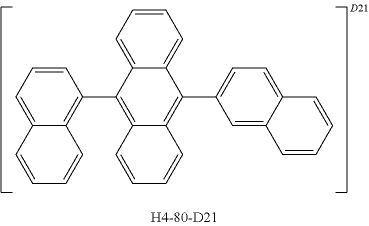 | 3.9 | 90 | 111 |
| Comparative | H1-2 | — | 4.0 | 81 | 67 |
| Example 1-1 | — | | | | |

### Device Example 2-1: Producing an OLED Depositing the Compound According to the Present Disclosure as a Single Host

An OLED was produced in the same manner as in Device Example 1-1, except that the compound shown in Table 2 below was used as a single host of the light-emitting layer.

### Comparative Examples 2-1 and 2-2: Producing an OLED Depositing a Comparative Compound as a Single Host

An OLED was produced in the same manner as in Device Example 2-1, except that the compound shown in Table 2 below was used as a single host of the light-emitting layer.

The driving voltage, conversion efficiency, and the time taken for luminance to decrease from 100% to 95% (lifetime; T₉₅) at a luminance of 1,000 nit of the OLEDs produced in Device Example 2-1 and Comparative Examples 2-1 and 2-2 are provided in Table 2 below. Herein, the conversion efficiency [Eff/Y] means the current efficiency [cd/A] divided by the Y coordinate value of CIE.

TABLE 2

| | | Driving | Conversion | |
|---|---|---|---|---|
| | | Voltage | Efficiency | Ligetime (T₉₅) |
| | Host | [V] | [Eff/Y] | [hr] |
| Device Example 2- 1 | 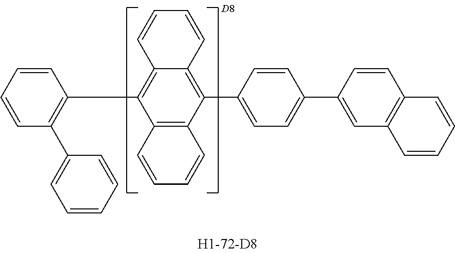 | 4.0 | 81 | 124 |
| Comparative | H1-2 | 4.0 | 81 | 67 |
| Example 2-1 | — | | | |
| Comparative Example 2-2 | 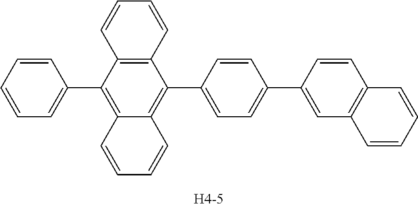 [SMILES: c1ccc(-c2c3ccccc3c(-c3ccc(-c4ccc5ccccc5c4)cc3)c3ccccc23)cc1] | 4.1 | 79 | 52 |

### Device Example 3-1: Producing a Tandem OLED Comprising the Compound According to the Present Disclosure as a Host

An OLED according to the present disclosure was produced. A transparent electrode indium tin oxide (ITO) thin film (10Ω/sq) on a glass substrate for an OLED (GEOMATEC CO., LTD.) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI was introduced into a cell of the vacuum vapor deposition apparatus, and Compound HT-1 was introduced into another cell of the vacuum vapor deposition apparatus. The two materials were evaporated at different rates, and Compound HI was deposited in a doping amount of 5 wt % based on the total amount of Compound HI and Compound HT-1 to form a hole injection layer having a thickness of 10 nm. Compound HT-1 was then deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 15 nm on the first hole transport layer. After forming the hole injection layer and the hole transport layers, a light-emitting layer was formed thereon as follows. Compound H1-2 was introduced into one cell of the vacuum vapor deposition apparatus as a host, and Compound BD was introduced into another cell as a dopant. The two materials were evaporated at a different rate, and the dopant was deposited in a doping amount of 2 wt % based on the total amount of the host and the dopant to form a first light-emitting layer having a thickness of 22.5 nm on the second hole transport layer. Compound ET-1 as a first hole-blocking layer material was then deposited on the first light-emitting layer to form a first hole-blocking layer having a thickness of 5 nm. On the first hole-blocking layer, a first electron transport layer having a thickness of 12 nm was deposited using Compound EI-1 as an electron transport layer material. Thereafter, Li was deposited in an amount of 1 wt % on Compound n-CGL to form a n-type charge generation layer having a thickness of 12 nm on the first electron transport layer. Compound HI was then introduced into a cell of the vacuum vapor deposition apparatus, and Compound HT-1 was introduced into another cell of the vacuum vapor deposition apparatus. The two materials were evaporated at different rates, and Compound HI was deposited in a doping amount of 15 wt % based on the total amount of Compound HI and Compound HT-1 to form a p-type charge generation layer having a thickness of 10 nm. Compound HT-1 was then deposited to a thickness of 80 nm to form a third hole transport layer, and then compound HT-2 was deposited to form a fourth hole transport layer having a thickness of 15 nm. A second light-emitting layer was then formed thereon as follows. Compound H1-2 was introduced into one cell of the vacuum vapor deposition apparatus as a host, and Compound BD was introduced into another cell as a dopant. The two materials were evaporated at a different rate, and the dopant was deposited in a doping amount of 2 wt % based on the total amount of the host and the dopant to form a second light-emitting layer having a thickness of 22.5 nm on the fourth hole transport layer. Compound ET-1 was deposited on the second light-emitting layer to form a second hole-blocking layer having a thickness of 5 nm. Compound EI-1 and Compound EI-2 were added to two other cells of the vacuum vapor deposition apparatus and evaporated at a ratio of 2:1 to deposit a second electron transport layer having a thickness of 25 nm. Compound Yb and Compound LiF were then added to two other cells and evaporated at a rate of 2:1 (Yb:LiF) to deposit an electron injection layer having a thickness of 1 nm on the second electron transport layer, and an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ Torr.

The driving voltage, conversion efficiency, and the time taken for luminance to decrease from 100% to 95% (lifetime; T₉₅) at a luminance of 1,000 nit of the OLED produced in Device Example 3-1 are provided in Table 3 below.

TABLE 3

| | | Driving | Conversion | |
|---|---|---|---|---|
| | | Voltage | Efficiency | Lifetime (T₉₅) |
| | Host | [V] | [Eff/Y] | [hr] |
| Device Example | H1-2 | 7.7 | 116 | 185 |
| 3-1 | | | | |

### Device Examples 5-1 to 5-6: Producing an OLED Comprising the Compound According to the Present Disclosure as Hosts

An OLED was produced in the same manner as in Device Example 1-1, except that the compounds shown in Table 4 below were used as hosts of the light-emitting layer.

### Comparative Examples 5-1 to 5-3: Producing an OLED Comprising a Comparative Compound as a Host

An OLED was produced in the same manner as in Device Example 5-1, except that the compound shown in Table 4 below was used as a host of the light-emitting layer.

The driving voltage, conversion efficiency, and the time taken for luminance to decrease from 100% to 95% (lifetime; T₉₅) at a luminance of 1,000 nit of the OLEDs produced in Device Examples 5-1 to 5-6 and Comparative Examples 5-1 to 5-3 are provided in Table 4 below. Herein, the conversion efficiency [Eff/Y] means the current efficiency [cd/A] divided by the Y coordinate value of CIE.

TABLE 4

| | | | Driving | Conversion | Lifetime |
|---|---|---|---|---|---|
| | | | Voltage | Efficiency | (T₉₅) |
| | First Host | Second Host | [V] | [Eff/Y] | [hr] |
| Device Example 5-1 | 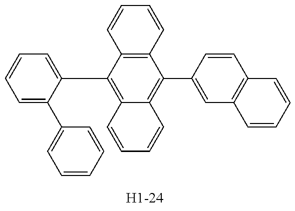 [SMILES: c1ccc(-c2ccccc2-c2c3ccccc3c(-c3ccc4ccccc4c3)c3ccccc23)cc1] | 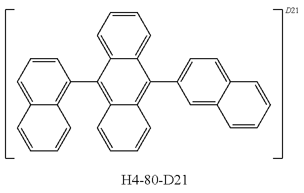 | 3.9 | 89 | 123 |
| Device Example 5-2 | 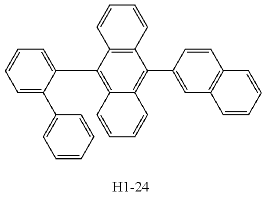 [SMILES: c1ccc(-c2ccccc2-c2c3ccccc3c(-c3ccc4ccccc4c3)c3ccccc23)cc1] | 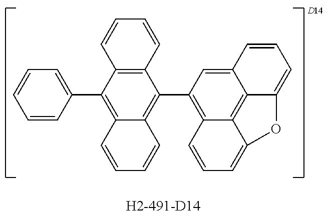 | 3.6 | 87 | 91 |
| Device Example 5-3 | 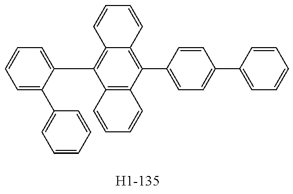 [SMILES: c1ccc(-c2ccc(-c3c4ccccc4c(-c4ccccc4-c4ccccc4)c4ccccc34)cc2)cc1] | 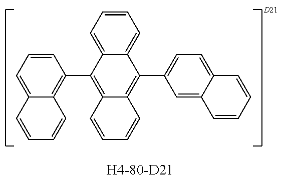 | 3.9 | 86 | 136 |
| Device Example 5-4 | 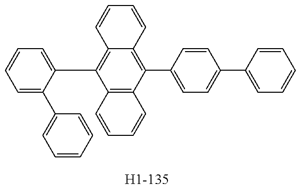 [SMILES: c1ccc(-c2ccc(-c3c4ccccc4c(-c4ccccc4-c4ccccc4)c4ccccc34)cc2)cc1] | 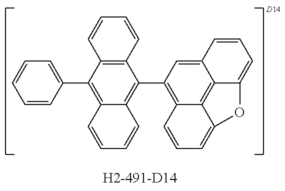 | 3.6 | 84 | 96 |
| Device Example 5-5 | 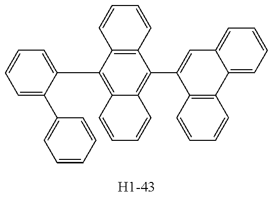 [SMILES: c1ccc(-c2ccccc2-c2c3ccccc3c(-c3cc4ccccc4c4ccccc34)c3ccccc23)cc1] | 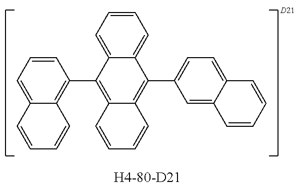 | 3.8 | 89 | 140 |
| Device Example 5-6 | 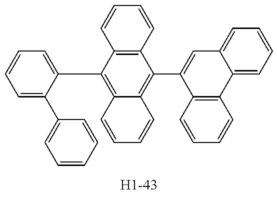 [SMILES: c1ccc(-c2ccccc2-c2c3ccccc3c(-c3cc4ccccc4c4ccccc34)c3ccccc23)cc1] | 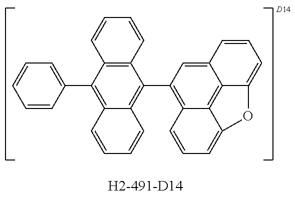 | 3.6 | 90 | 120 |
| Compara- tive Example 5-1 | 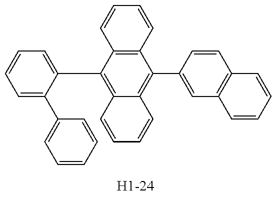 [SMILES: c1ccc(-c2ccccc2-c2c3ccccc3c(-c3ccc4ccccc4c3)c3ccccc23)cc1] | — | 4.0 | 83 | 91 |
| Compara- tive Example 5-2 | 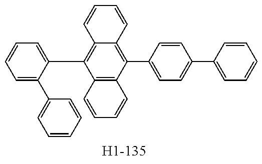 [SMILES: c1ccc(-c2ccc(-c3c4ccccc4c(-c4ccccc4-c4ccccc4)c4ccccc34)cc2)cc1] | — | 4.0 | 78 | 71 |
| Compara- tive Example 5-3 | 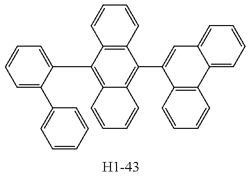 [SMILES: c1ccc(-c2ccccc2-c2c3ccccc3c(-c3cc4ccccc4c4ccccc34)c3ccccc23)cc1] | — | 3.9 | 87 | 90 |

From Tables 1 to 4 above, it can be confirmed that the organic electroluminescent device comprising the compound or a specific combination of compounds according to the present disclosure as a host material(s) exhibits significantly improved lifetime properties while exhibiting an equivalent or lower driving voltage and/or equivalent or higher current efficiency compared to the organic electroluminescent device comprising the comparative compound as a host material.

The compounds used in the above Device Examples and Comparative Examples are shown in Table 5 below.

TABLE 5

| | |
|---|---|
| Hole Injection Layer/ Hole Transport Layer/ Charge Generation Layer | 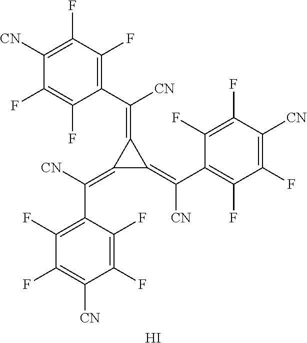 |
| | 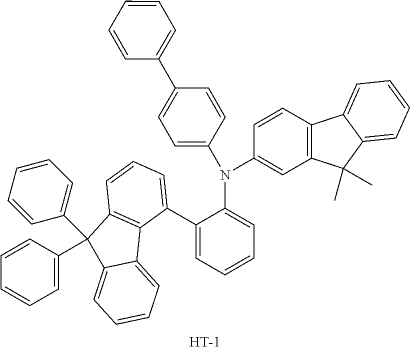 |
| | 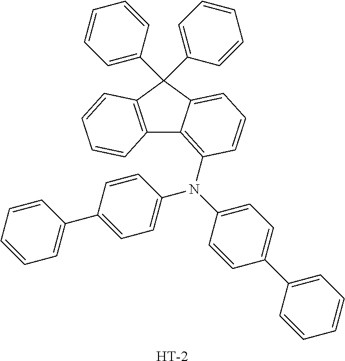 |
| | 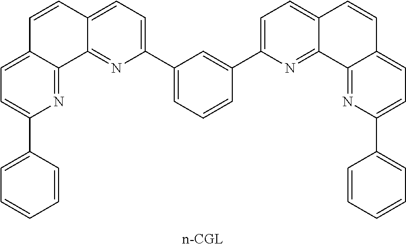 [SMILES: c1ccc(-c2ccc3ccc4ccc(-c5cccc(-c6ccc7ccc8ccc(-c9ccccc9)nc8c7n6)c5)nc4c3n2)cc1] |
| Light-Emitting Layer | 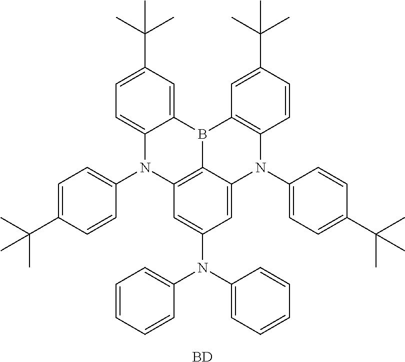 |
| Electron Buffer Layer/ Hole-Blocking Layer | 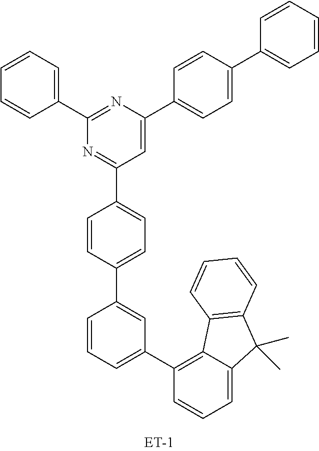 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| Electron Transport Layer/ Electron Injection Layer | 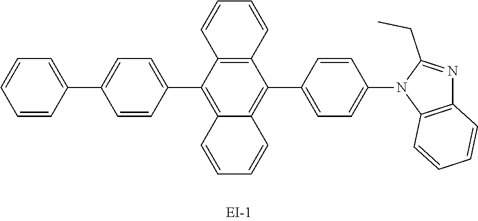 [SMILES: CCc1nc2ccccc2n1-c1ccc(-c2c3ccccc3c(-c3ccc(-c4ccccc4)cc3)c3ccccc23)cc1] |
| | 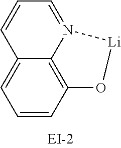 |

1. A plurality of host materials comprising at least one first host compound and at least one second host compound, wherein the first host compound is represented by the following Formula 1, the second host compound is represented by the following Formula 2, and the first host compound and the second compound are different from each other: 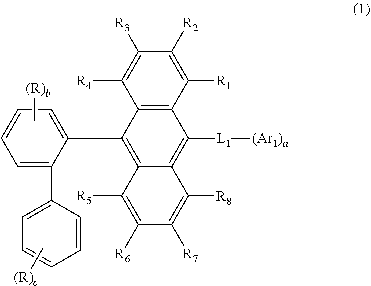
in Formula 1,
L₁ represents a single bond, a phenylene unsubstituted or substituted with deuterium, a naphthylene unsubstituted or substituted with deuterium, or a phenanthrenylene unsubstituted or substituted with deuterium;
Ar₁ each independently represents a substituted or unsubstituted (C₆-C₃₀)aryl, or a substituted or unsubstituted (3- to 30-membered)heteroaryl;
R and R₁ to R₈ each independently represent hydrogen or deuterium; and
a represents an integer of 1 or 2, b represents an integer of 4, and c represents an integer of 5; and each Ar₁ may be the same as or different from each other;
with a proviso that Formula 1 is not represented as 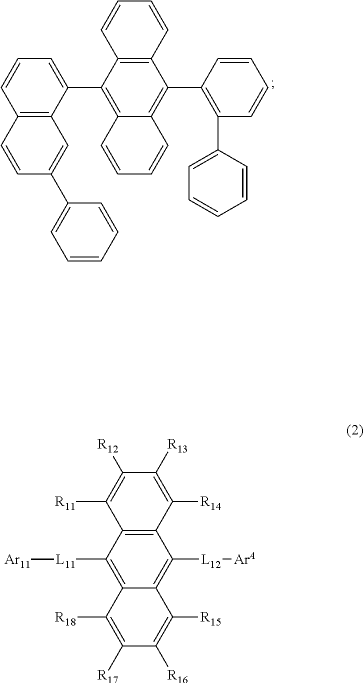
in Formula 2,
Ar^{A} represents a substituted or unsubstituted (C₆-C₃₀)aryl, a substituted or unsubstituted (13- to 30-membered)heteroaryl, or the following Formula A-1: 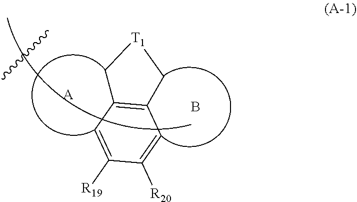
wherein T₁ represents O, S, or CRₐR_{b};
Ring A and Ring B each independently represent a substituted or unsubstituted (C₆-C₃₀)arene, or a substituted or unsubstituted (3- to 30-membered) heteroarene;
Ar₁₁ represents a substituted or unsubstituted (C₆-C₃₀)aryl, or a substituted or unsubstituted (13- to 30-membered)heteroaryl;
R₁₁ to R₁₈ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄);
R₁₉ and R₂₀ each independently represent the position linked to L₁₂, or represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered) heteroaryl, a substituted or unsubstituted (C₃-C₃₀)cycloalkyl, a substituted or unsubstituted (C₃-C₃₀)cycloalkenyl, a substituted or unsubstituted (3- to 7-membered) heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄);
Rₐ and R_{b} each independently represent a substituted or unsubstituted (C₁-C₃₀)alkyl, or a substituted or unsubstituted (C₆-C₃₀)aryl, or may be linked to each other to form a ring(s);
L₁₁ to L₁₃ each independently represent a single bond, a substituted or unsubstituted (C₆-C₃₀)arylene, or a substituted or unsubstituted (3- to 30-membered) heteroarylene; and
Ar₁₃ and Ar₁₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, or a substituted or unsubstituted fused ring group of a (C₃-C₃₀) aliphatic ring(s) and a (C₆-C₃₀) aromatic ring(s).

2. The plurality of host materials according to claim 1, wherein Ar₁ each independently represents a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof.

3. The plurality of host materials according to claim 1, wherein Ar₁₁ and Ar^{A} each independently are selected from a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof.

4. The plurality of host materials according to claim 1, wherein Ar₁₁ is selected from a phenyl unsubstituted or substituted with deuterium, a biphenyl unsubstituted or substituted with deuterium, a terphenyl unsubstituted or substituted with deuterium, a naphthyl unsubstituted or substituted with deuterium, a phenanthrenyl unsubstituted or substituted with deuterium, or a combination thereof; and Ar^{A} represents a dibenzofuranyl unsubstituted or substituted with deuterium, or a dibenzothiophenyl unsubstituted or substituted with deuterium.

5. The plurality of host materials according to claim 1, wherein Ar^{A} is represented by the following Formula B-1: 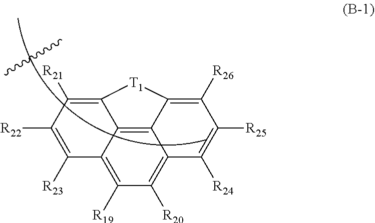
in Formula B-1,
R₂₁ to R₂₆ each independently represent the position linked to L₁₂, or represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered) heteroaryl, a substituted or unsubstituted (C₃-C₃₀)cycloalkyl, a substituted or unsubstituted (C₃-C₃₀)cycloalkenyl, a substituted or unsubstituted (3- to 7-membered) heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s); and
T₁, R₁₉, R₂₀, L₁₃, Ar₁₃, and Ar₁₄ are as defined in claim 1.

6. The plurality of host materials according to claim 1, wherein Formula 2 is represented by the following Formula 2-1 or 2-2: 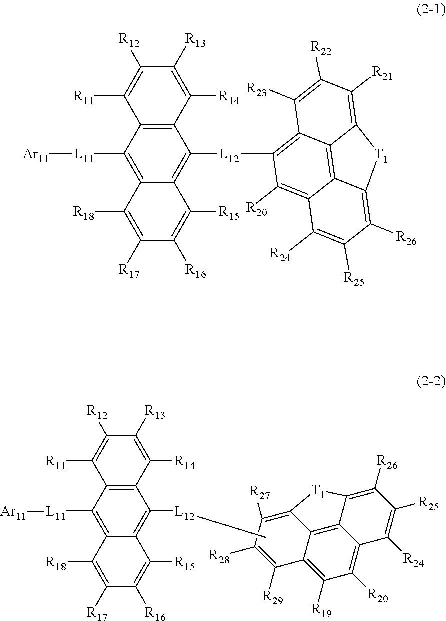
in Formulas 2-1 and 2-2,
R₂₁ to R₂₆ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s);
R₂₇ to R₂₉ each independently represent the position linked to L₁₂, or represent hydrogen, deuterium, a halogen, a cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring group of a (C₃-C₃₀) aliphatic ring(s) and a (C₆-C₃₀) aromatic ring(s), or -L₁₃-N(Ar₁₃)(Ar₁₄), or may be linked to the adjacent substituent to form a ring(s); and
T₁, R₁₁ to R₂₀, L₁₁ to L₁₃, Ar₁₁, Ar₁₃, and Ar₁₄ are as defined in claim 1.

7. The plurality of host materials according to claim 1, wherein at least one of Formula 1 and Formula 2 contains deuterium.

8. The plurality of host materials according to claim 1, wherein the substituted alkyl, the substituted alkenyl, the substituted aryl, the substituted arylene, the substituted heteroaryl, the substituted heteroarylene, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted fused ring group of an aliphatic ring(s) and an aromatic ring(s), the substituted arene, and the substituted heteroarene each independently are substituted with at least one selected from the group consisting of deuterium, a halogen, a cyano, carboxyl, nitro, hydroxyl, phosphine oxide, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered) heterocycloalkyl, (C₆-C₃₀)aryloxy, (C₆-C₃₀)arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, amino, mono- or di(C₁-C₃₀)alkylamino, mono- or di(C₂-C₃₀)alkenylamino, mono- or di(C₆-C₃₀)arylamino, mono- or di(3- to 30-membered) heteroarylamino, (C₁-C₃₀)alkyl(C₂-C₃₀)alkenylamino, (C₁-C₃₀)alkyl(C₆-C₃₀)arylamino, (C₁-C₃₀)alkyl(3- to 30-membered)heteroarylamino, (C₂-C₃₀)alkenyl(C₆-C₃₀)arylamino, (C₂-C₃₀)alkenyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, di(C₆-C₃₀)arylboronyl, (C₆-C₃₀)arylphosphinyl, di(C₁-C₃₀)alkylboronyl, (C₁-C₃₀)alkyl(C₆-C₃₀)arylboronyl, (C₆-C₃₀)aryl(C₁-C₃₀)alkyl, (C₁-C₃₀)alkyl(C₆-C₃₀)aryl, and a combination thereof, which may be further substituted with deuterium.

9. The plurality of host materials according to claim 1, wherein the compound represented by Formula 1 is at least one selected from the following compounds: 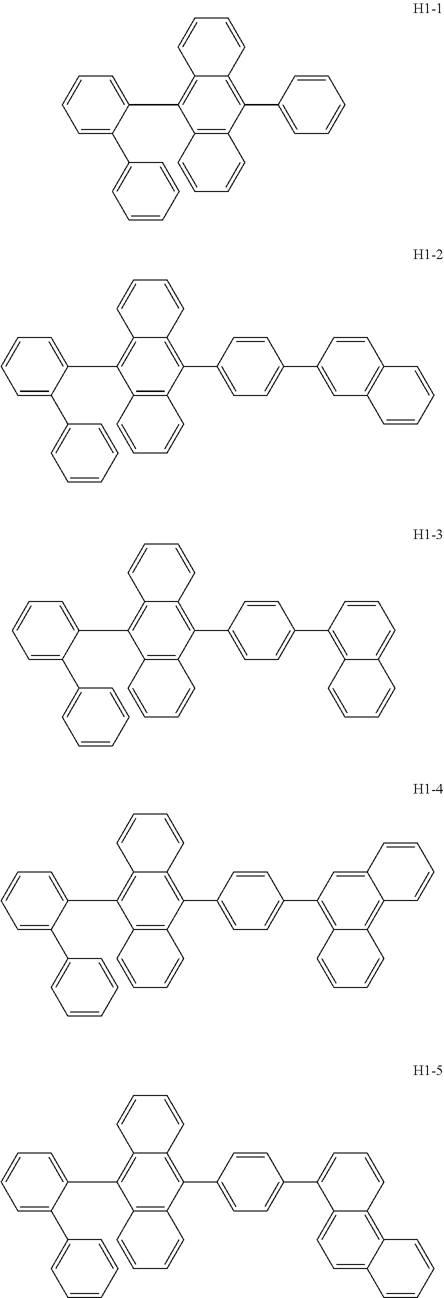 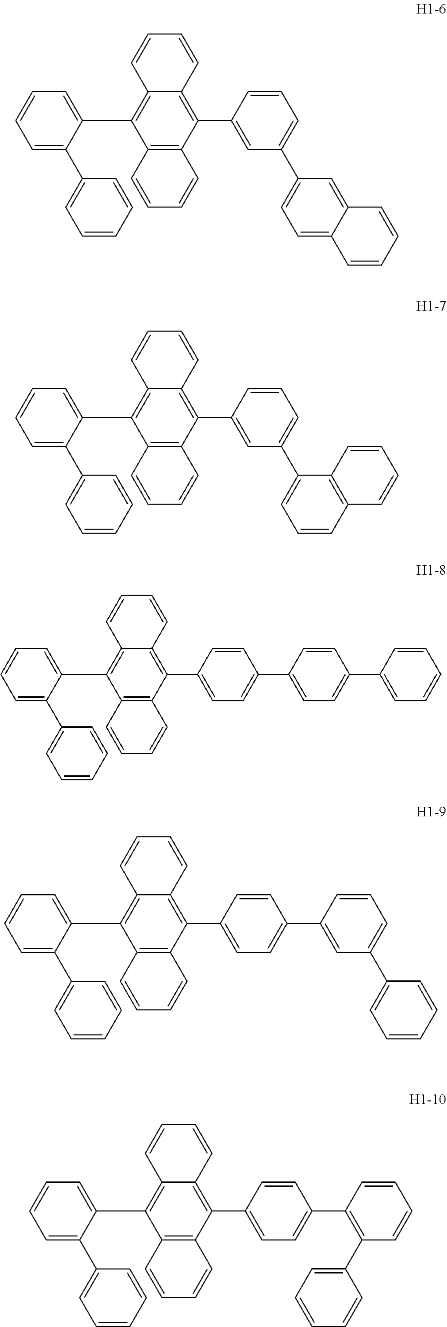 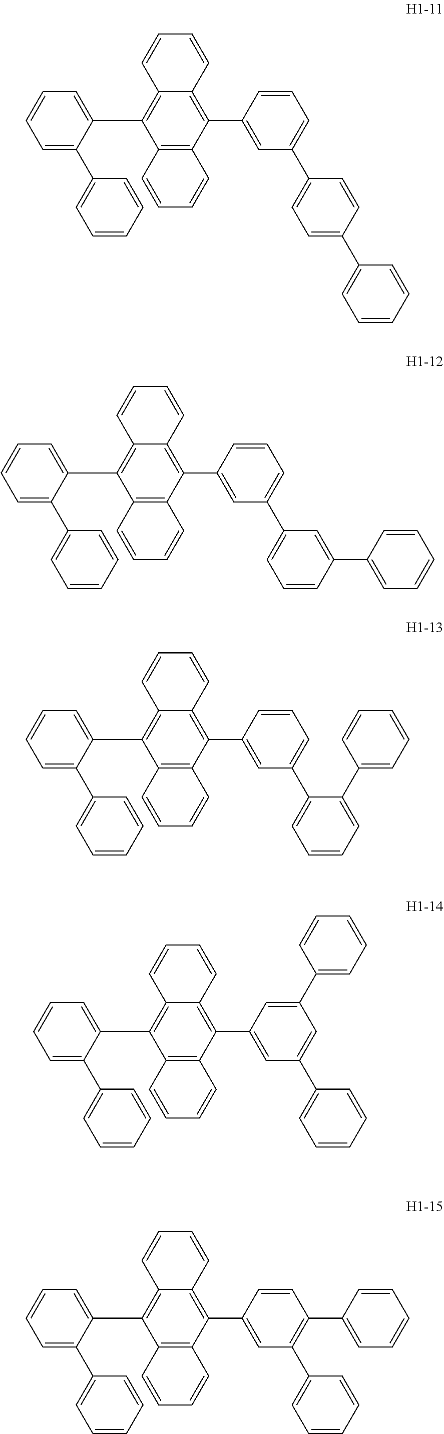 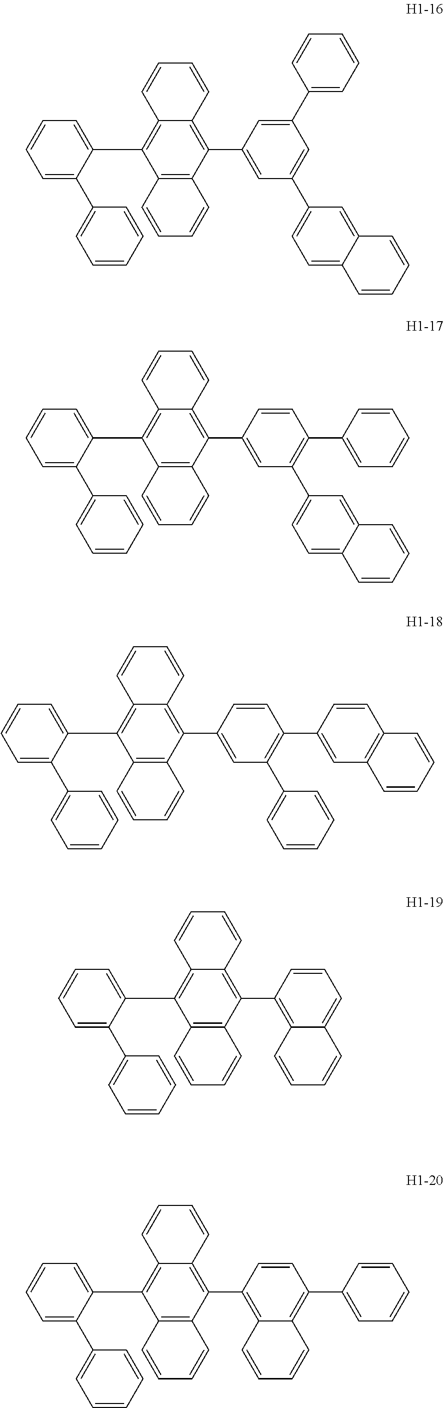 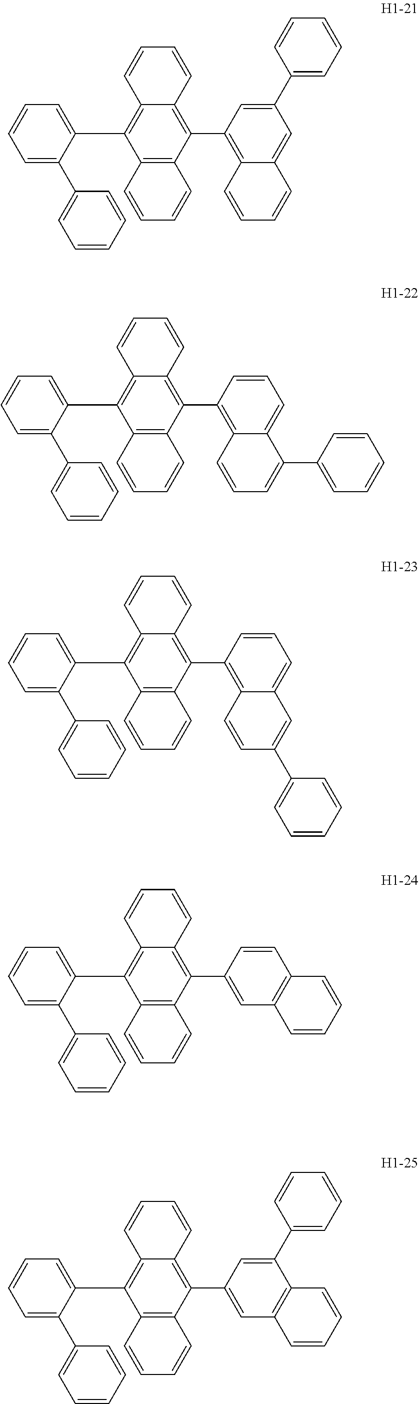 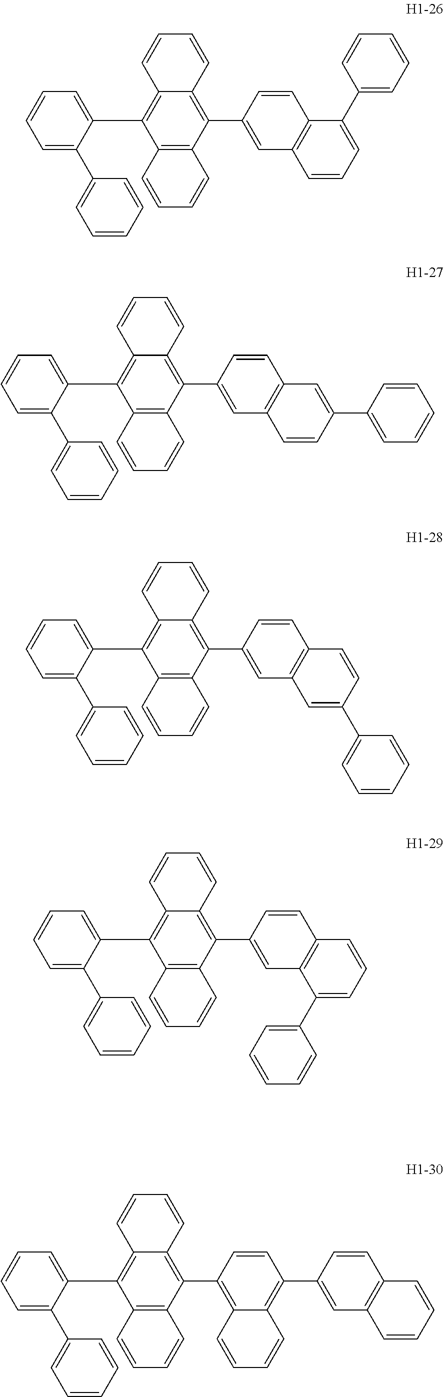 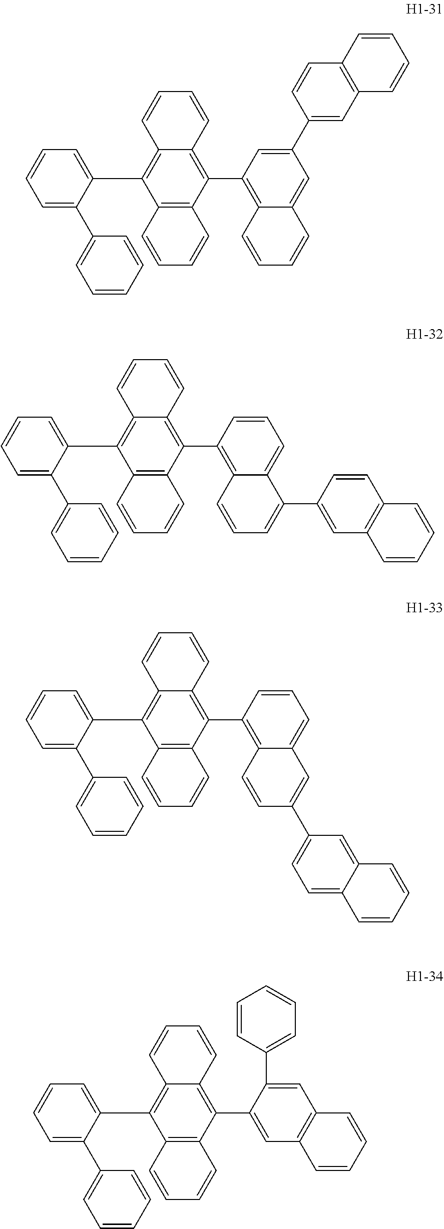 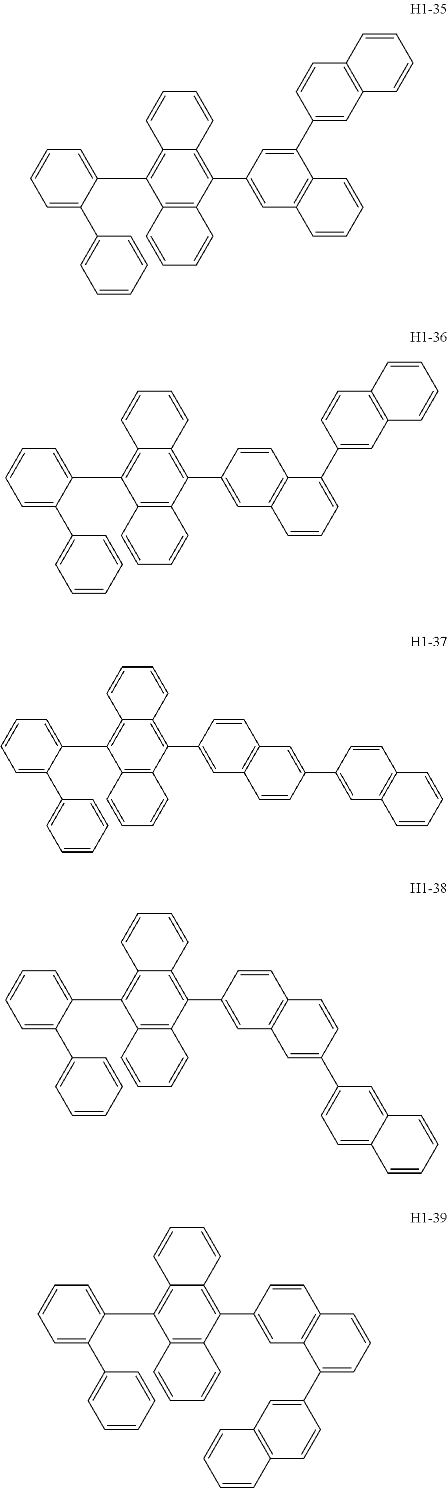 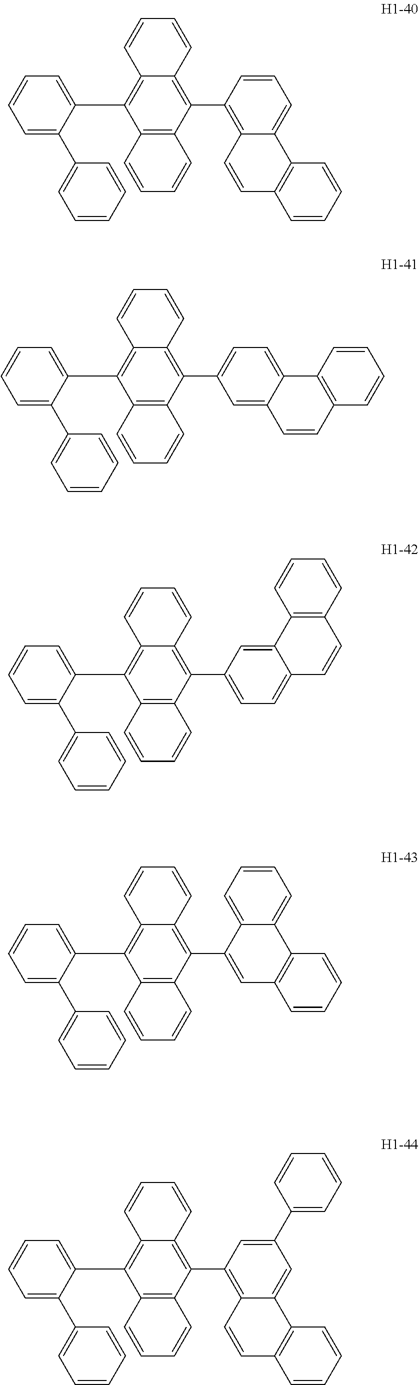 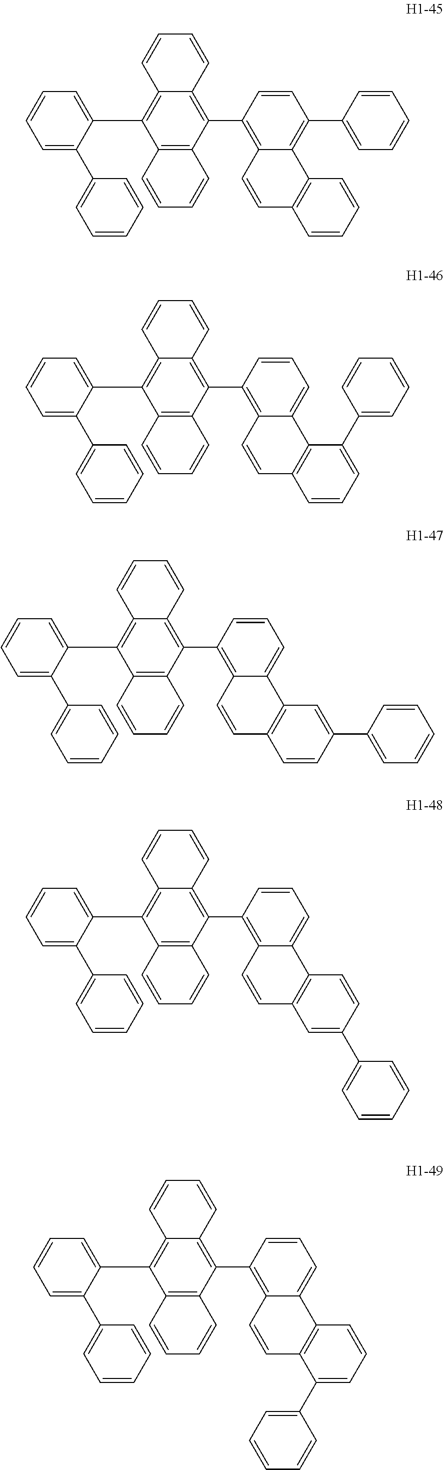 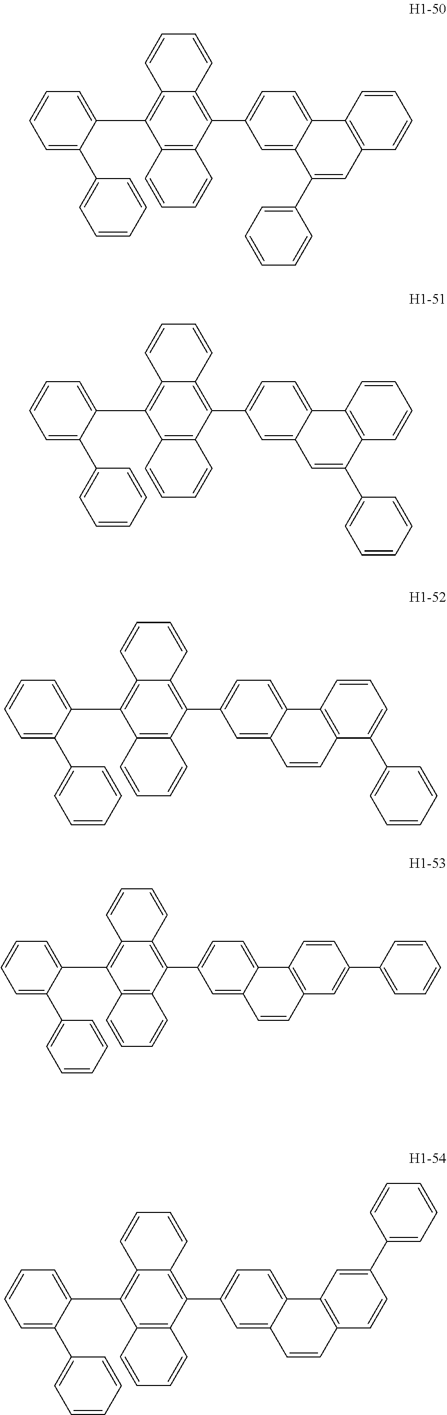 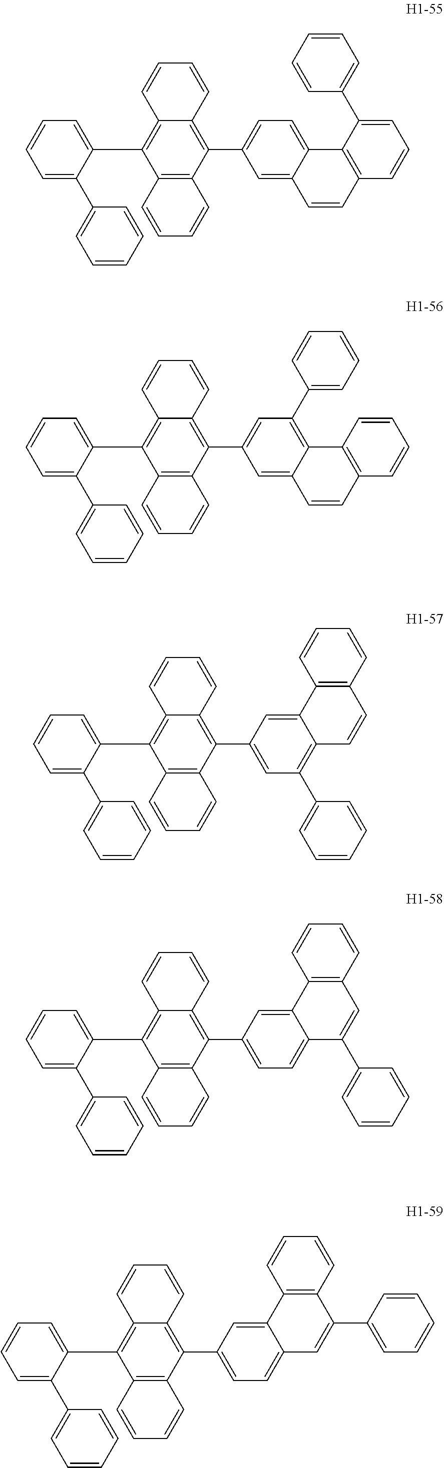 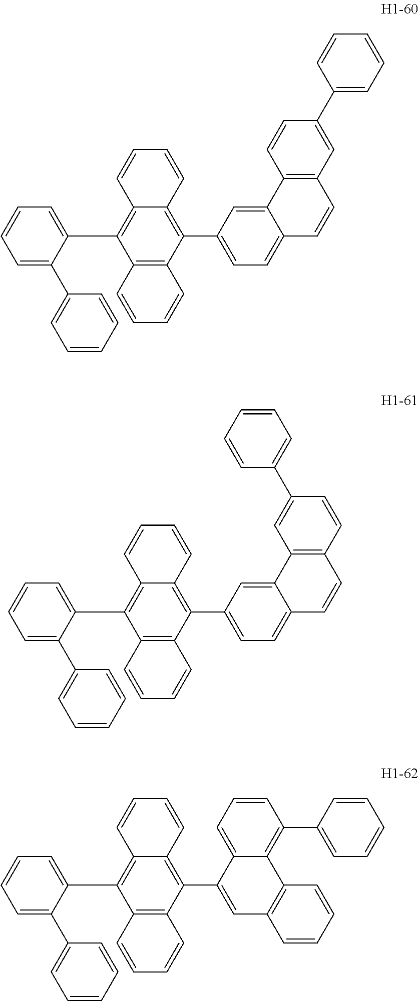 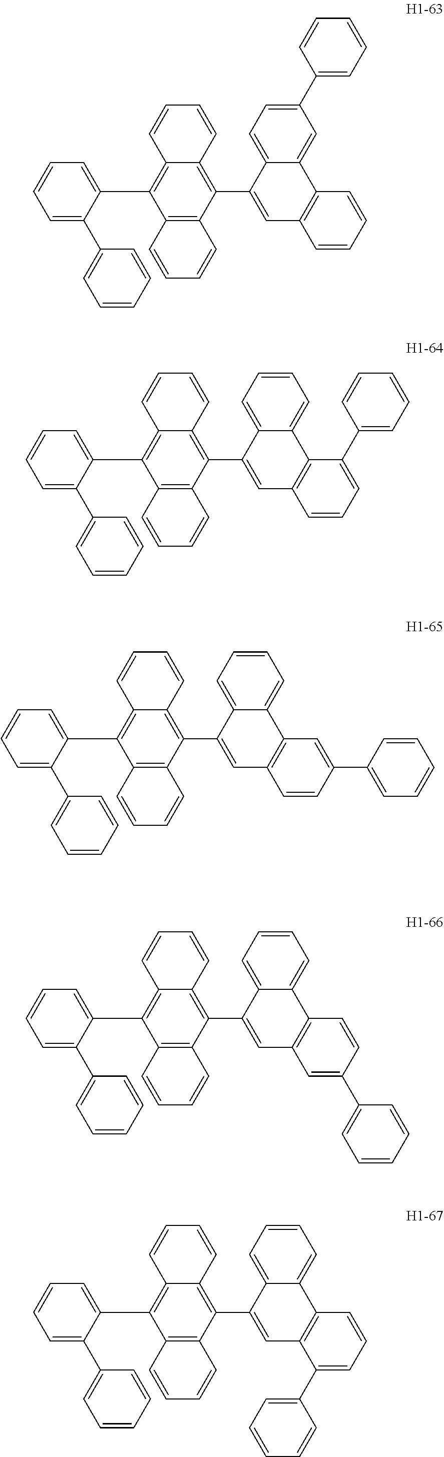 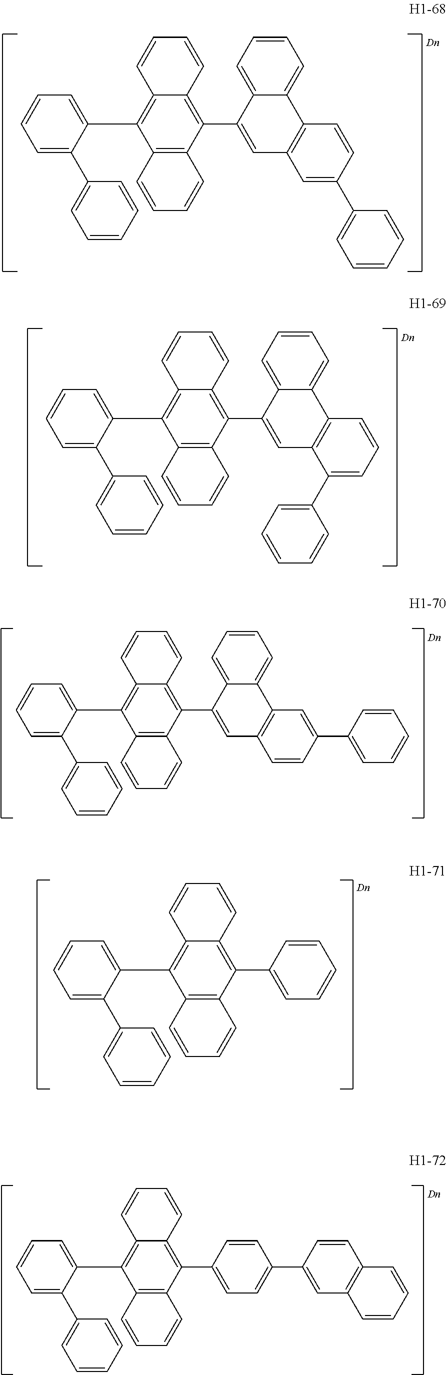 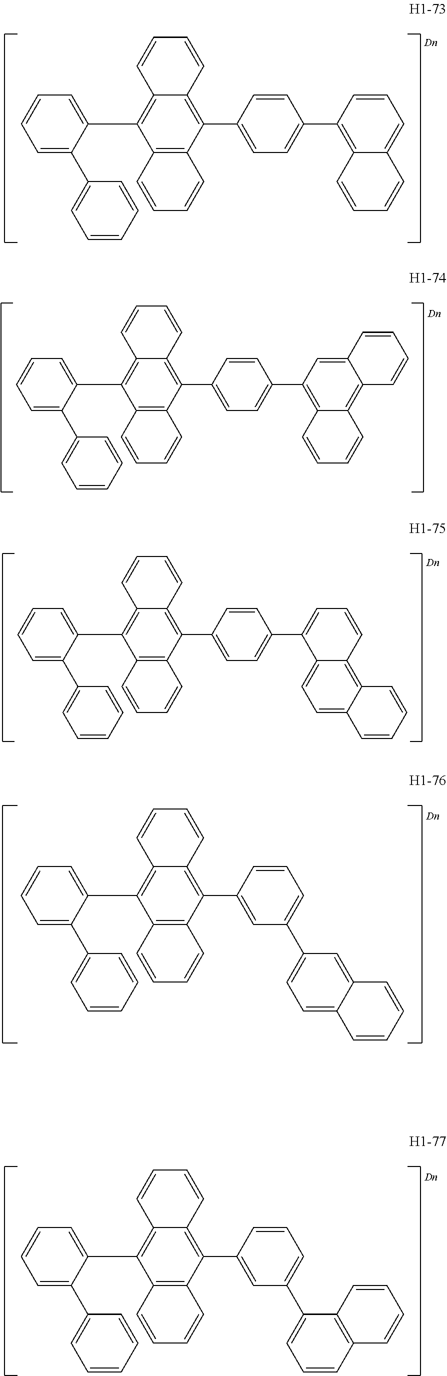 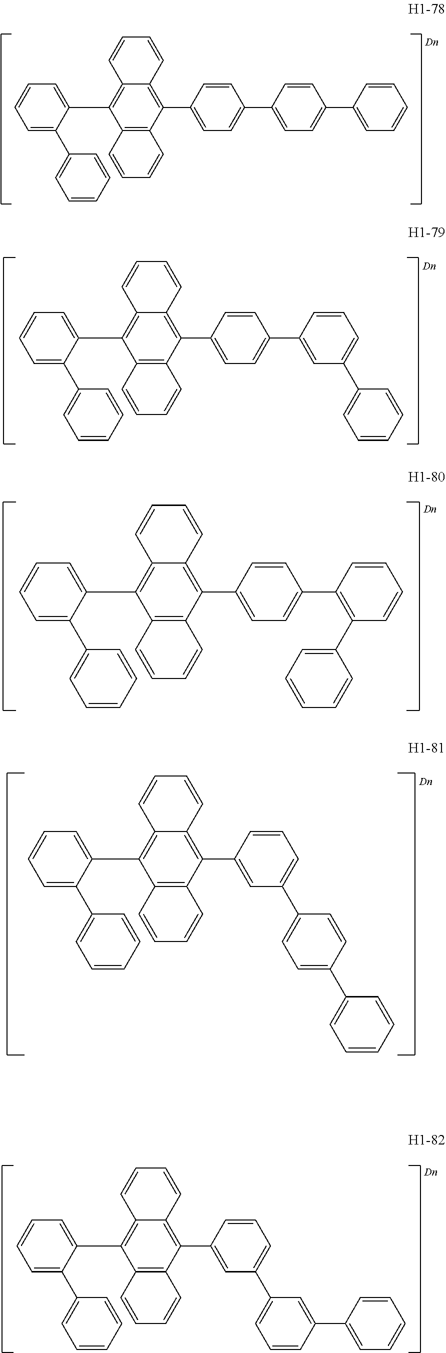 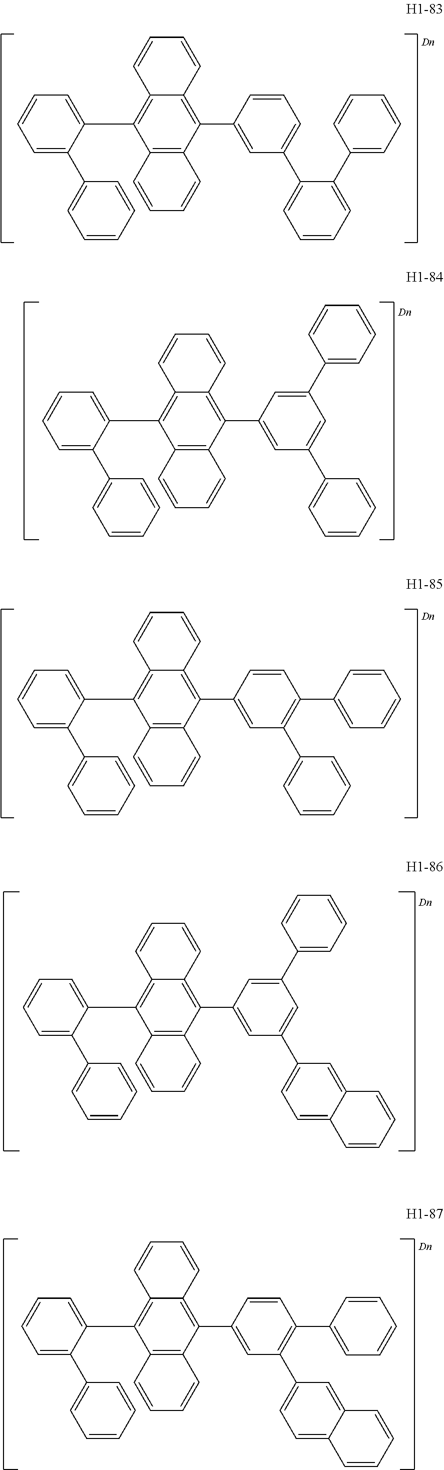 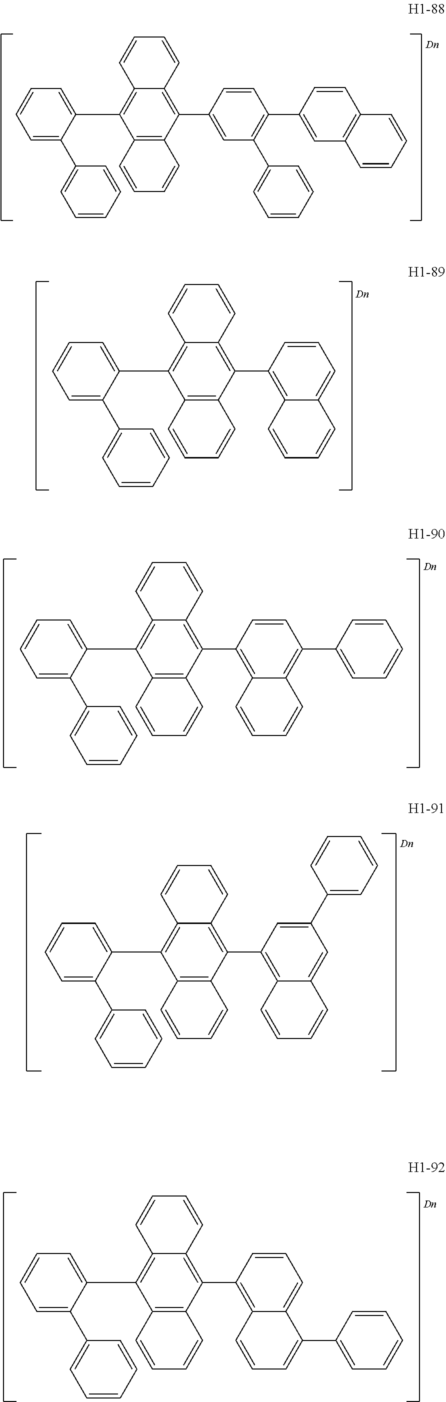 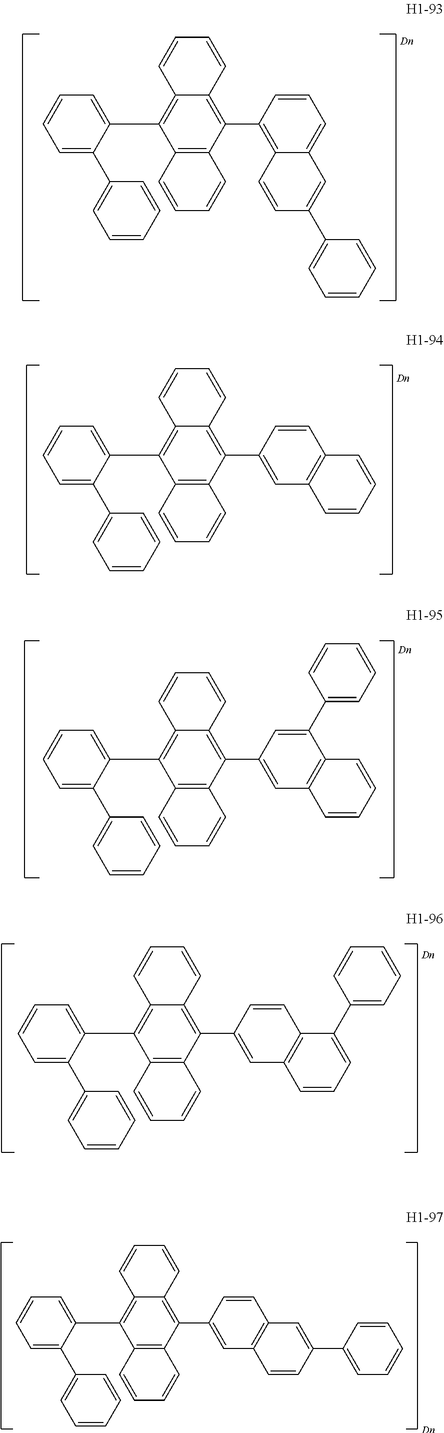 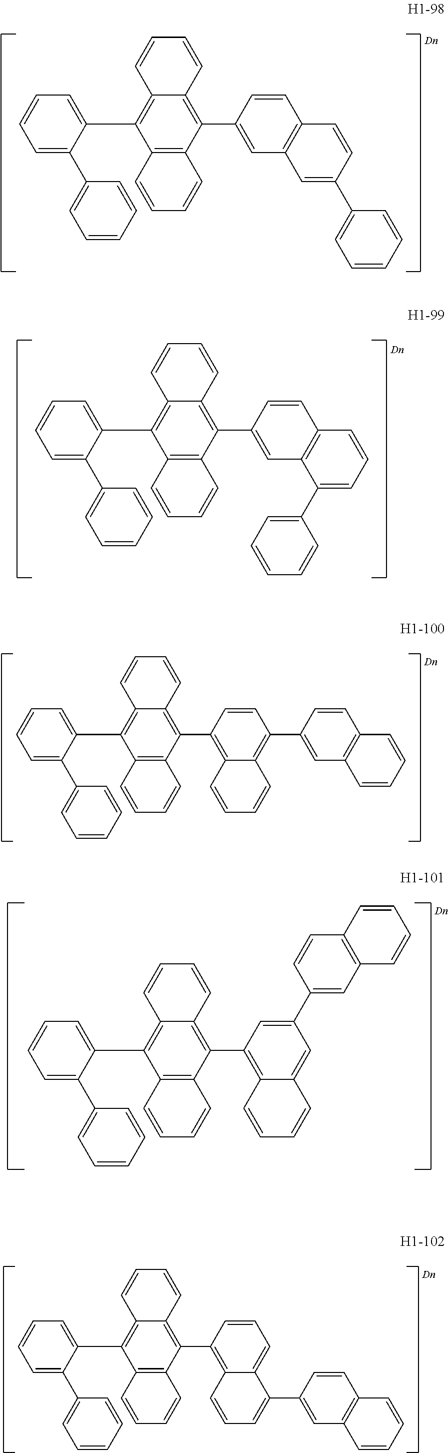 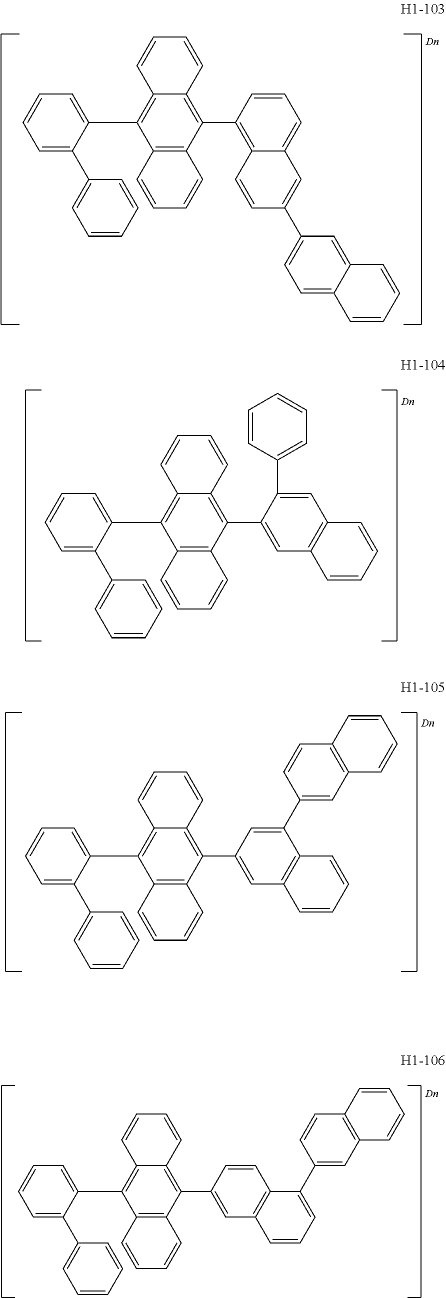 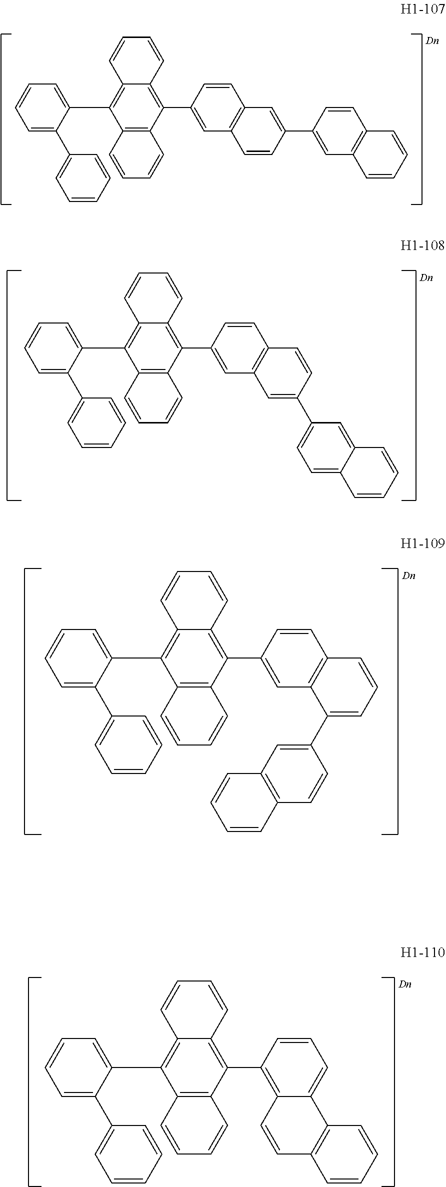 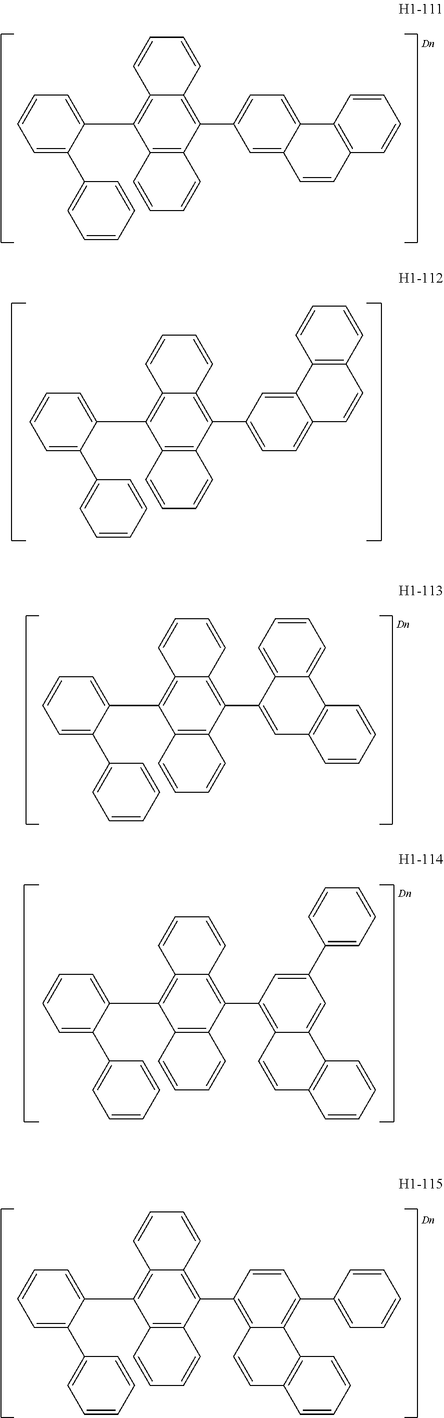 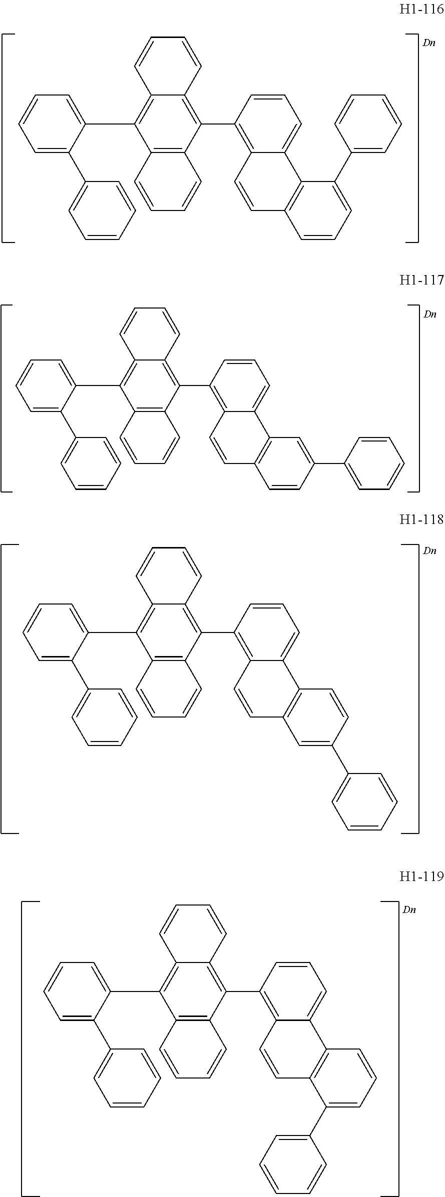 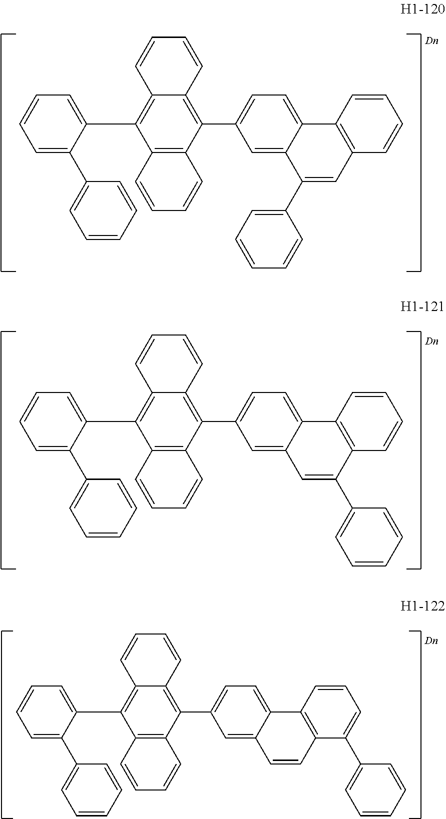 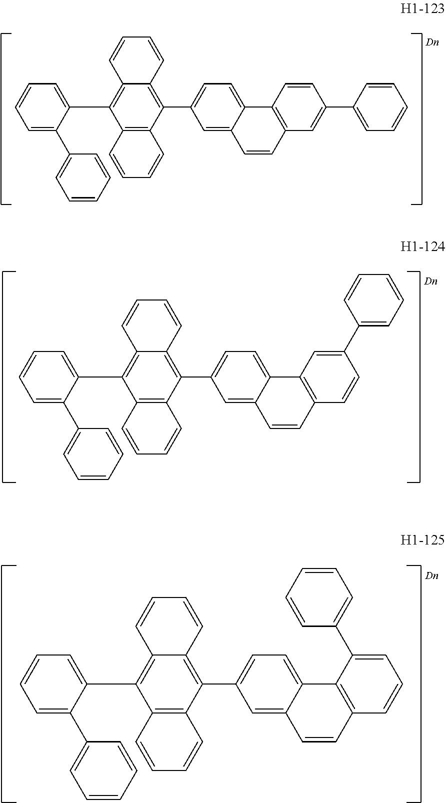 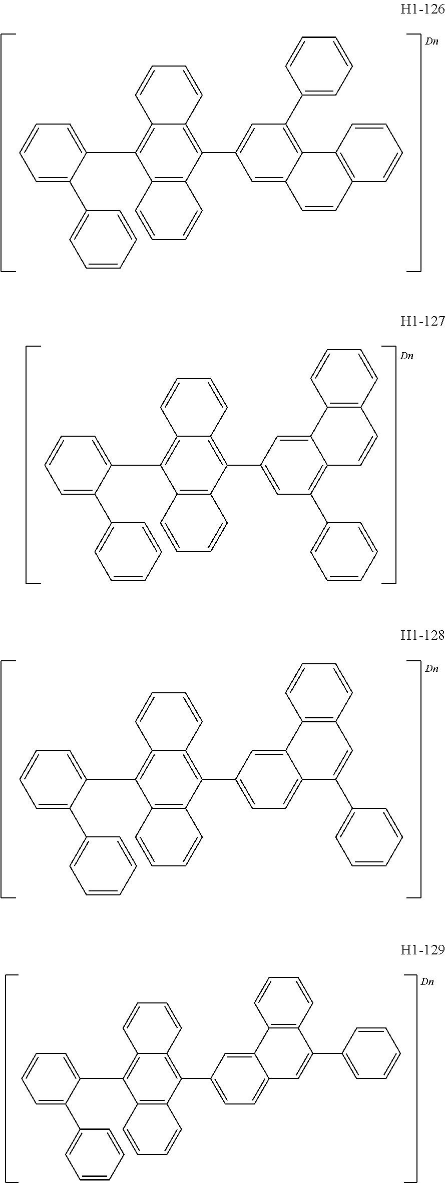 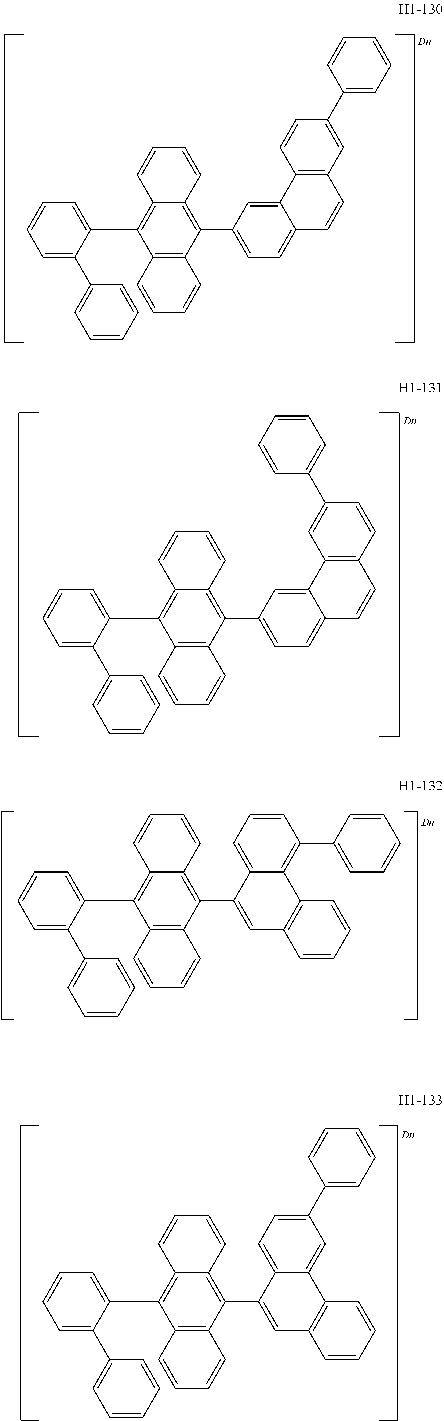 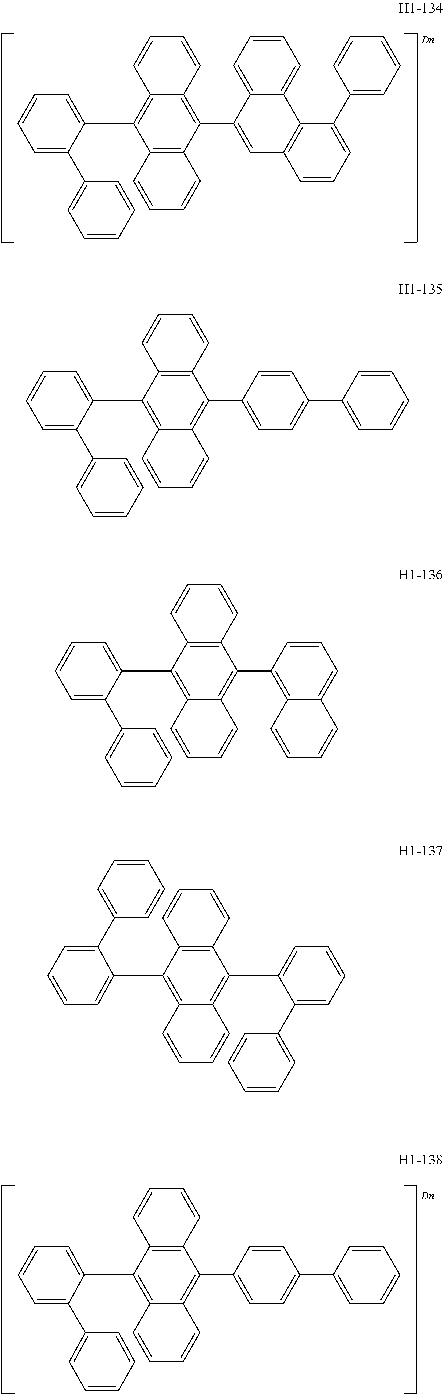 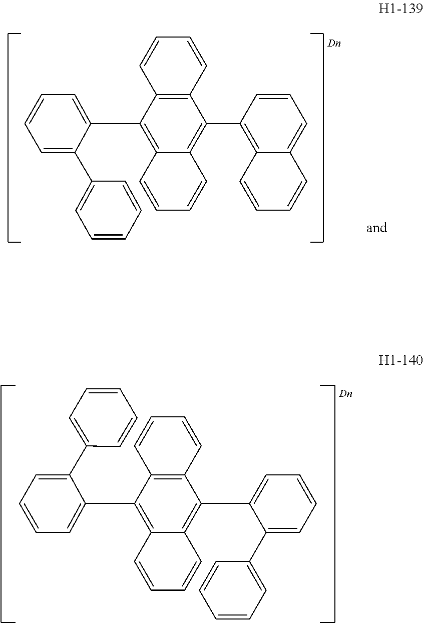
wherein Dₙ signifies that n hydrogens are replaced with deuterium, and n is an integer from 1 to the maximum number of hydrogens in the compound.

10. The plurality of host materials according to claim 1, wherein the compound represented by Formula 2 is at least one selected from the following compounds: 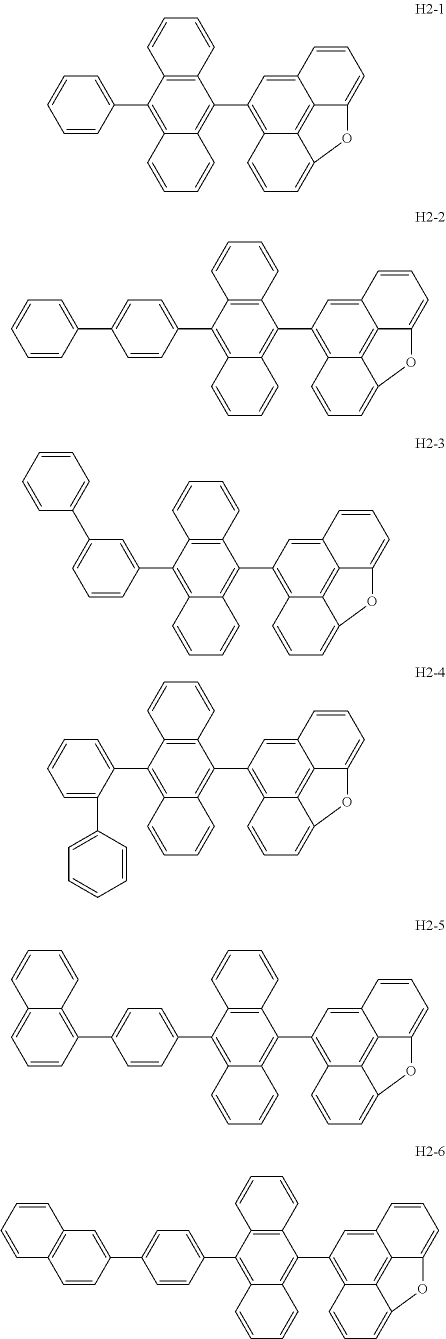 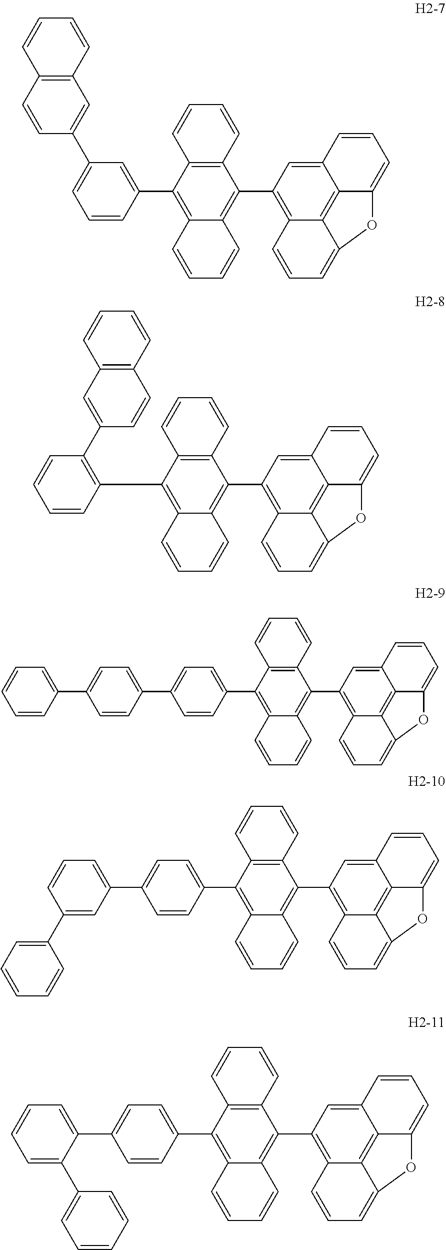 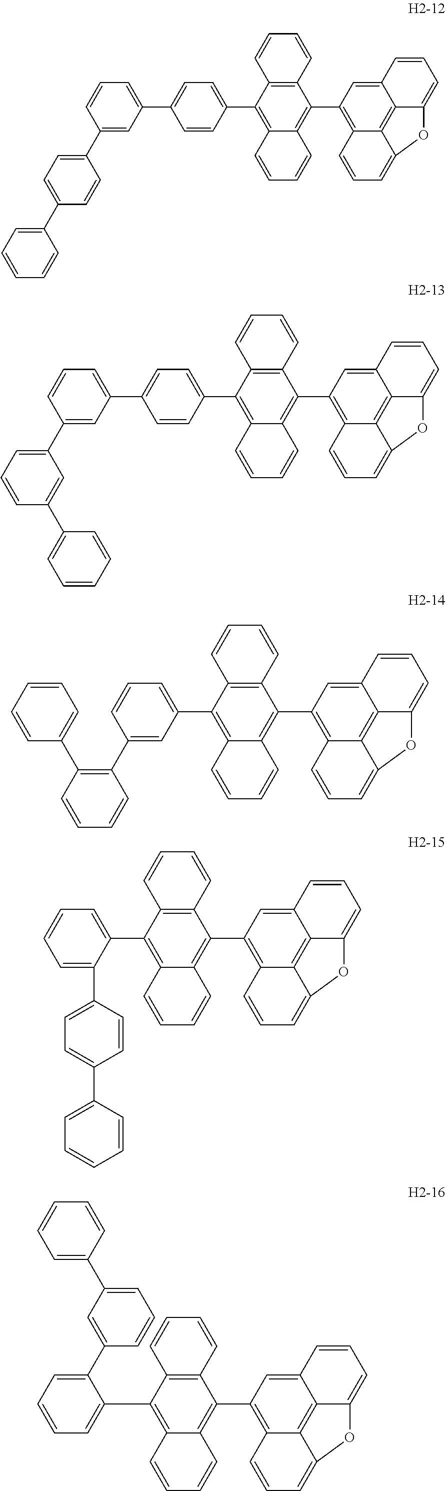 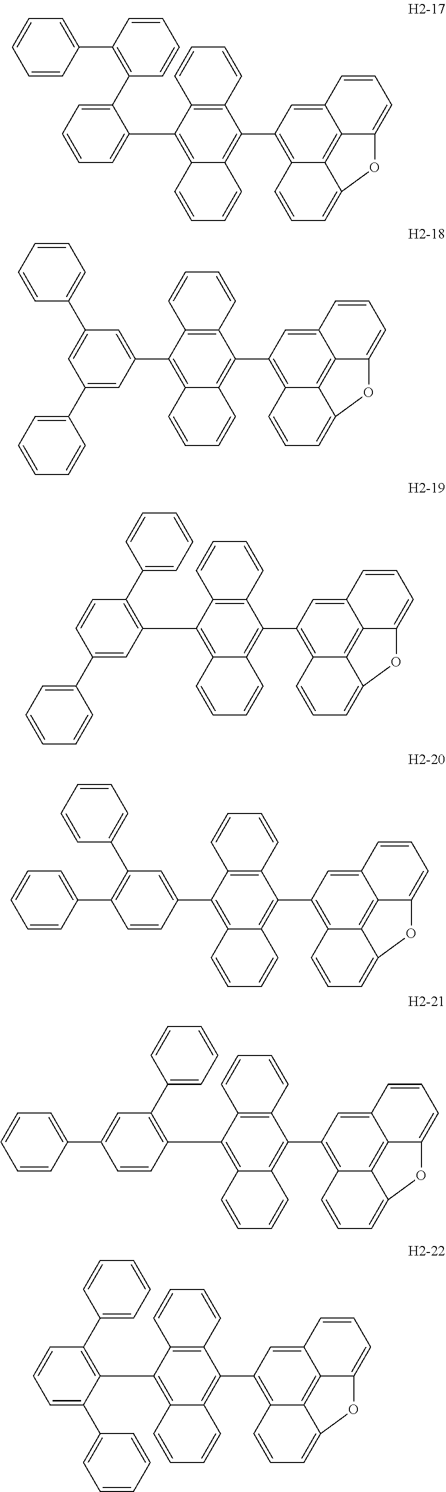 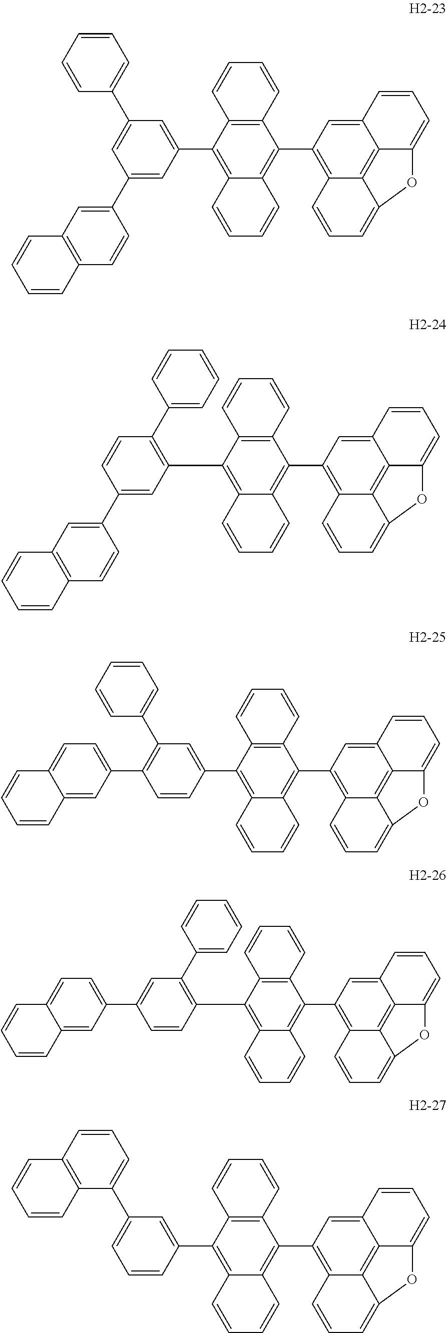 [SMILES: c1ccc(-c2cc(-c3ccc4ccccc4c3)cc(-c3c4ccccc4c(-c4cc5cccc6oc7cccc4c7c56)c4ccccc34)c2)cc1 ; c1ccc(-c2ccc(-c3ccc4ccccc4c3)cc2-c2c3ccccc3c(-c3cc4cccc5oc6cccc3c6c45)c3ccccc23)cc1 ; c1ccc(-c2cc(-c3c4ccccc4c(-c4cc5cccc6oc7cccc4c7c56)c4ccccc34)ccc2-c2ccc3ccccc3c2)cc1 ; c1ccc(-c2cc(-c3ccc4ccccc4c3)ccc2-c2c3ccccc3c(-c3cc4cccc5oc6cccc3c6c45)c3ccccc23)cc1 ; c1cc(-c2cccc3ccccc23)cc(-c2c3ccccc3c(-c3cc4cccc5oc6cccc3c6c45)c3ccccc23)c1] 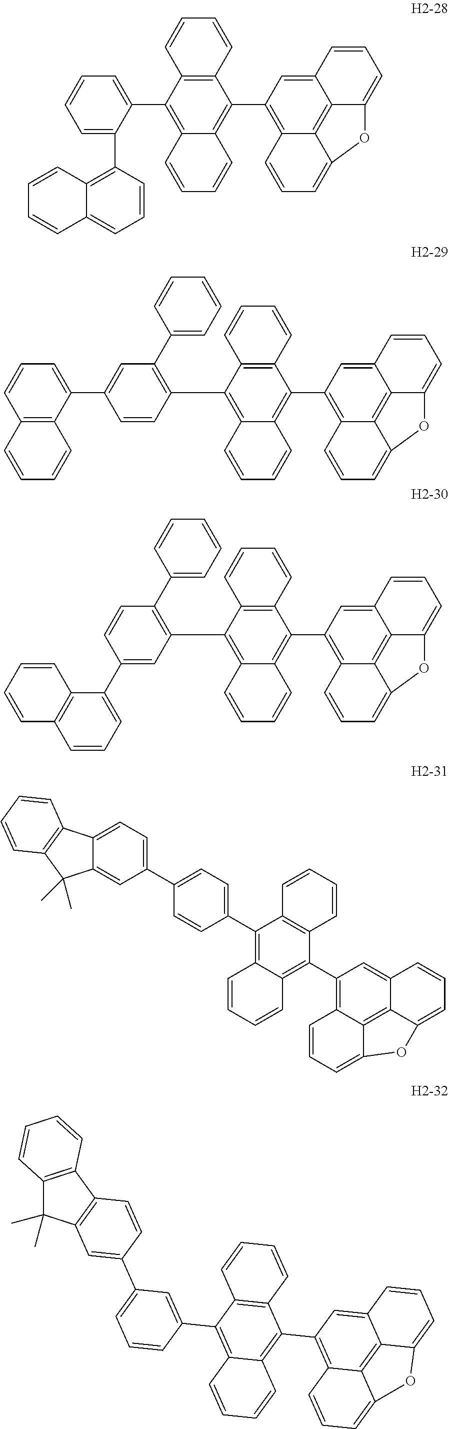 [SMILES: c1ccc(-c2c3ccccc3c(-c3cc4cccc5oc6cccc3c6c45)c3ccccc23)c(-c2cccc3ccccc23)c1 ; c1ccc(-c2cc(-c3cccc4ccccc34)ccc2-c2c3ccccc3c(-c3cc4cccc5oc6cccc3c6c45)c3ccccc23)cc1 ; c1ccc(-c2ccc(-c3cccc4ccccc34)cc2-c2c3ccccc3c(-c3cc4cccc5oc6cccc3c6c45)c3ccccc23)cc1 ; CC1(C)c2ccccc2-c2ccc(-c3ccc(-c4c5ccccc5c(-c5cc6cccc7oc8cccc5c8c67)c5ccccc45)cc3)cc21 ; CC1(C)c2ccccc2-c2ccc(-c3cccc(-c4c5ccccc5c(-c5cc6cccc7oc8cccc5c8c67)c5ccccc45)c3)cc21] 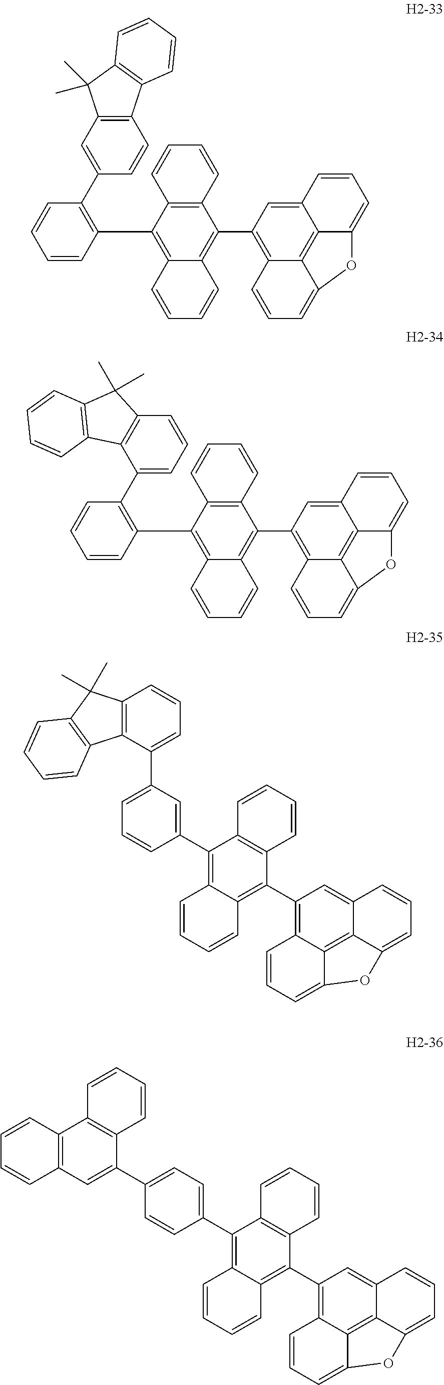 [SMILES: CC1(C)c2ccccc2-c2ccc(-c3ccccc3-c3c4ccccc4c(-c4cc5cccc6oc7cccc4c7c56)c4ccccc34)cc21 ; CC1(C)c2ccccc2-c2c(-c3ccccc3-c3c4ccccc4c(-c4cc5cccc6oc7cccc4c7c56)c4ccccc34)cccc21 ; CC1(C)c2ccccc2-c2c(-c3cccc(-c4c5ccccc5c(-c5cc6cccc7oc8cccc5c8c67)c5ccccc45)c3)cccc21 ; c1ccc2c(c1)cc(-c1ccc(-c3c4ccccc4c(-c4cc5cccc6oc7cccc4c7c56)c4ccccc34)cc1)c1ccccc12] 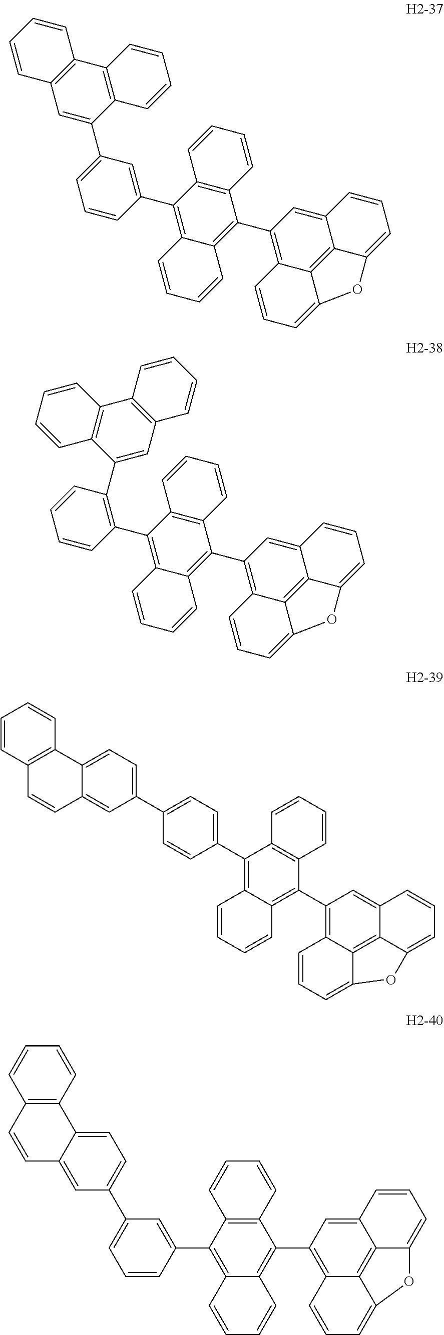 [SMILES: c1cc(-c2c3ccccc3c(-c3cc4cccc5oc6cccc3c6c45)c3ccccc23)cc(-c2cc3ccccc3c3ccccc23)c1 ; c1ccc(-c2cc3ccccc3c3ccccc23)c(-c2c3ccccc3c(-c3cc4cccc5oc6cccc3c6c45)c3ccccc23)c1 ; c1ccc2c(c1)ccc1cc(-c3ccc(-c4c5ccccc5c(-c5cc6cccc7oc8cccc5c8c67)c5ccccc45)cc3)ccc12 ; c1cc(-c2ccc3c(ccc4ccccc43)c2)cc(-c2c3ccccc3c(-c3cc4cccc5oc6cccc3c6c45)c3ccccc23)c1] 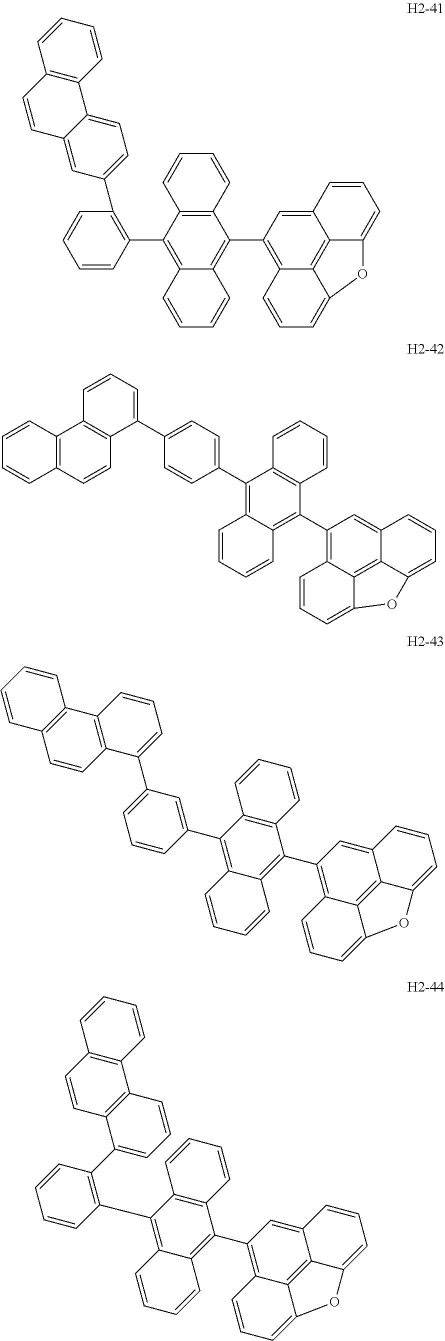 [SMILES: c1ccc(-c2c3ccccc3c(-c3cc4cccc5oc6cccc3c6c45)c3ccccc23)c(-c2ccc3c(ccc4ccccc43)c2)c1 ; c1ccc2c(c1)ccc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2ccc2ccccc23)c1 ; c1ccc(-c2cccc3c2ccc2ccccc23)c(-c2c3ccccc3c(-c3cc4cccc5oc6cccc3c6c45)c3ccccc23)c1] 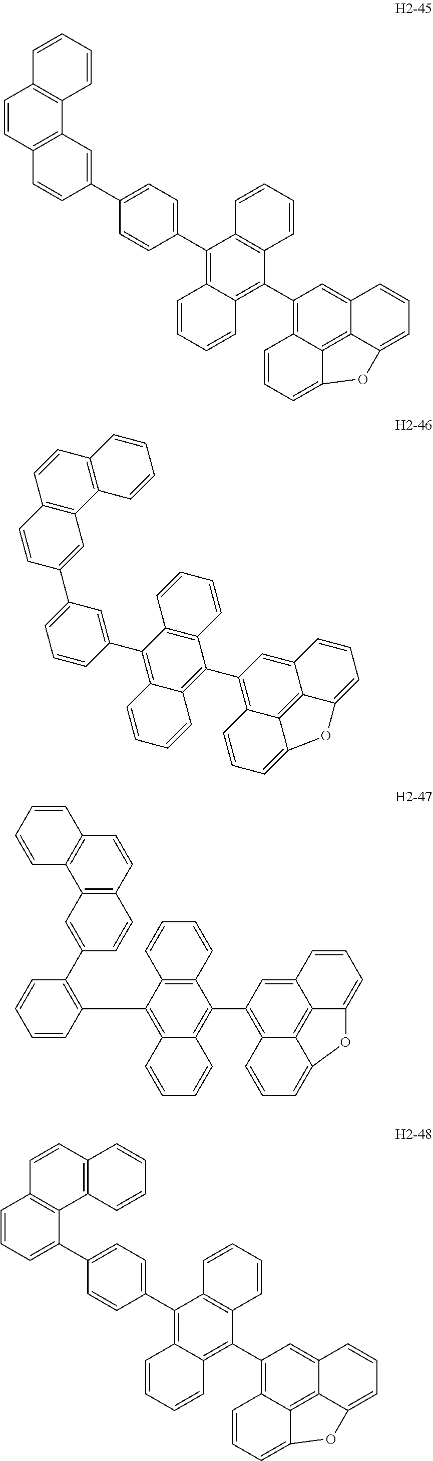 [SMILES: c1ccc2c(c1)ccc1ccc(-c3ccc(-c4c5ccccc5c(-c5cc6cccc7oc8cccc5c8c67)c5ccccc45)cc3)cc12 ; c1cc(-c2ccc3ccc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3ccc4ccccc4c3c2)c1 ; c1ccc2c(c1)ccc1cccc(-c3ccc(-c4c5ccccc5c(-c5cc6cccc7oc8cccc5c8c67)c5ccccc45)cc3)c12] 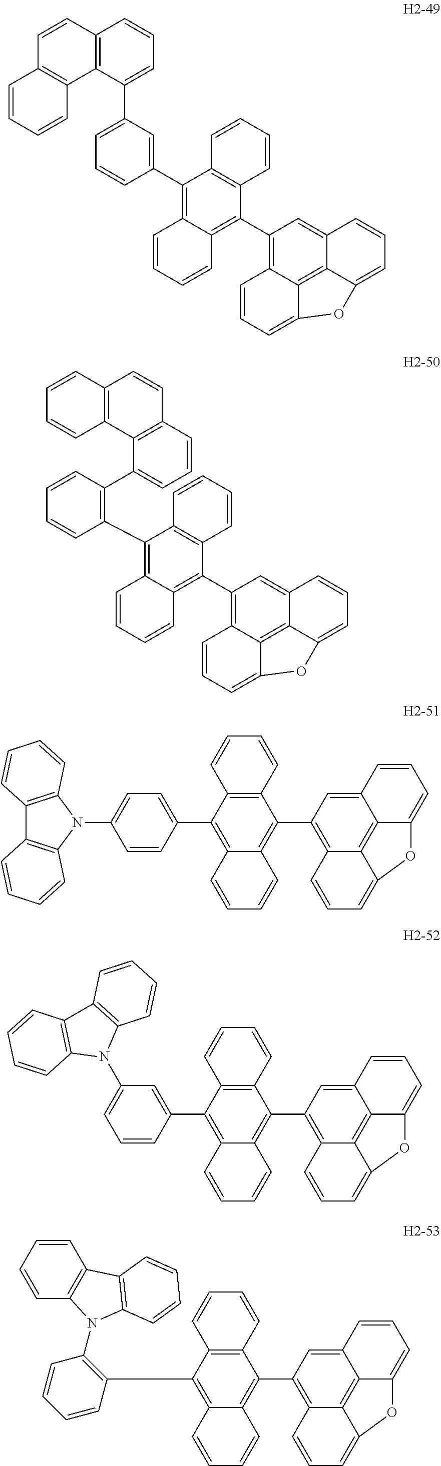 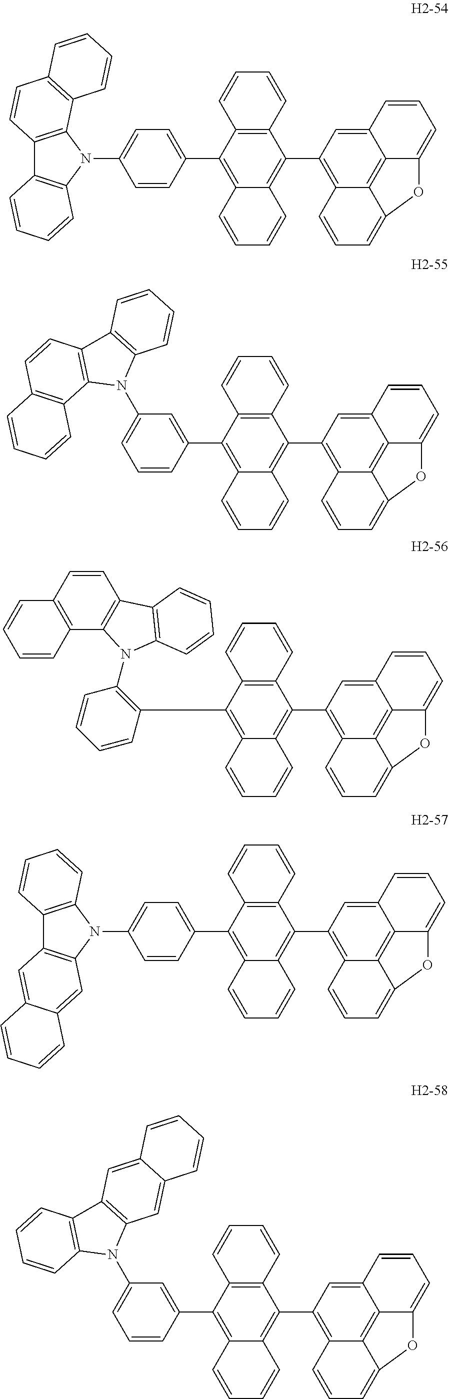 [SMILES: c1ccc2c(c1)ccc1c3ccccc3n(-c3ccc(-c4c5ccccc5c(-c5cc6cccc7oc8cccc5c8c67)c5ccccc45)cc3)c21 ; c1cc(-c2c3ccccc3c(-c3cc4cccc5oc6cccc3c6c45)c3ccccc23)cc(-n2c3ccccc3c3ccc4ccccc4c32)c1 ; c1ccc(-n2c3ccccc3c3ccc4ccccc4c32)c(-c2c3ccccc3c(-c3cc4cccc5oc6cccc3c6c45)c3ccccc23)c1 ; c1ccc2cc3c(cc2c1)c1ccccc1n3-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3cc4ccccc4cc32)c1] 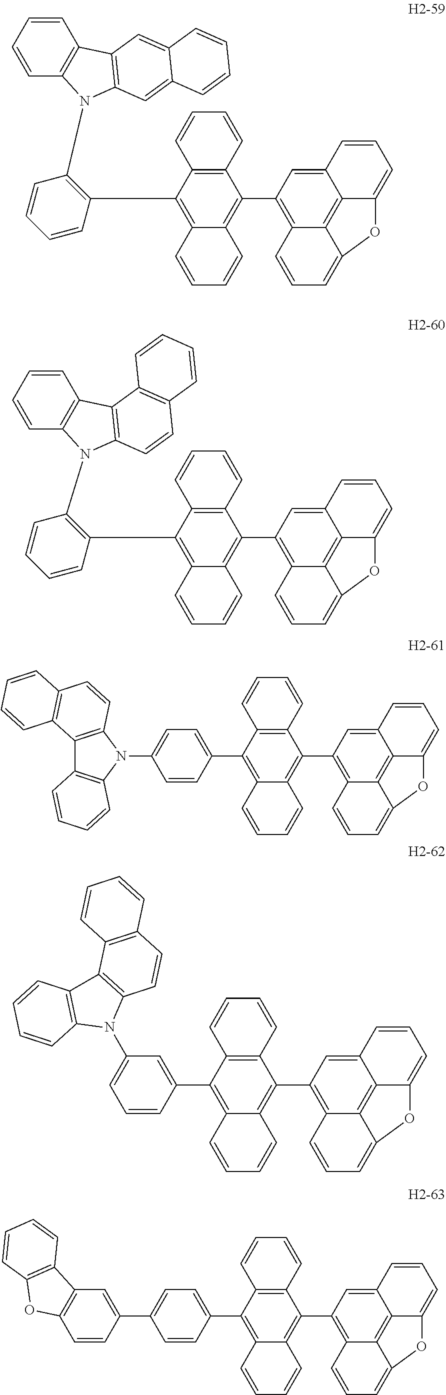 [SMILES: c1ccc(-n2c3ccccc3c3cc4ccccc4cc32)c(-c2c3ccccc3c(-c3cc4cccc5oc6cccc3c6c45)c3ccccc23)c1 ; c1ccc(-n2c3ccccc3c3c4ccccc4ccc32)c(-c2c3ccccc3c(-c3cc4cccc5oc6cccc3c6c45)c3ccccc23)c1 ; c1ccc2c(c1)ccc1c2c2ccccc2n1-c1ccc(-c2c3ccccc3c(-c3cc4cccc5oc6cccc3c6c45)c3ccccc23)cc1 ; c1cc(-c2c3ccccc3c(-c3cc4cccc5oc6cccc3c6c45)c3ccccc23)cc(-n2c3ccccc3c3c4ccccc4ccc32)c1 ; c1ccc2c(c1)oc1ccc(-c3ccc(-c4c5ccccc5c(-c5cc6cccc7oc8cccc5c8c67)c5ccccc45)cc3)cc12] 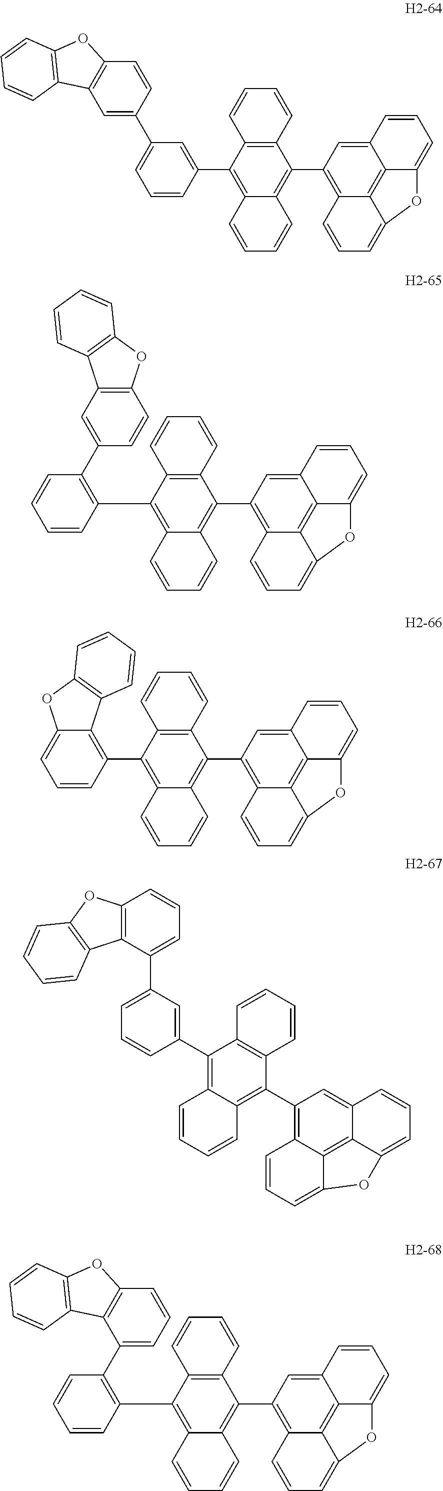 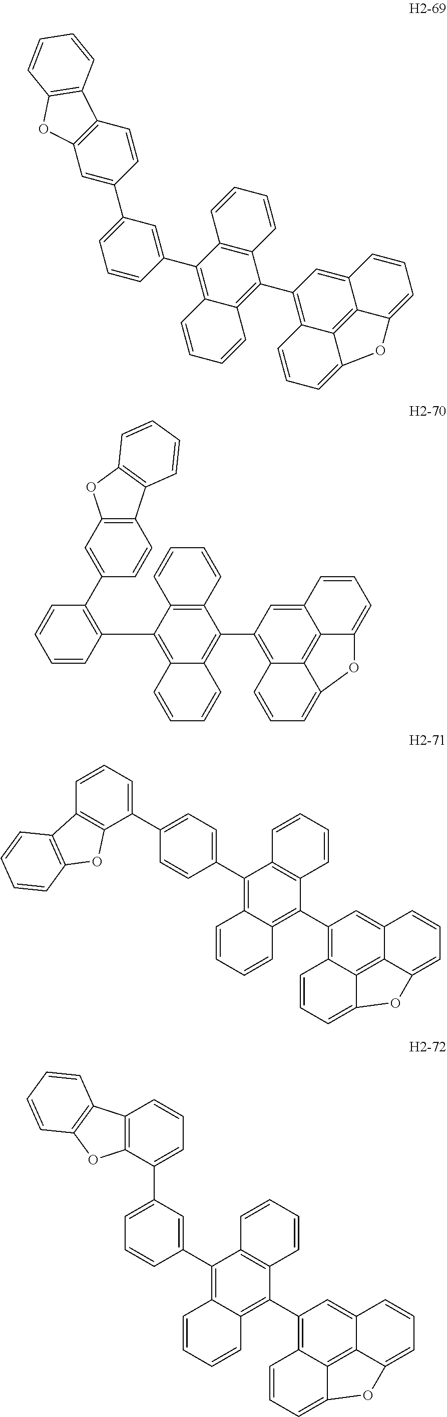 [SMILES: c1cc(-c2ccc3c(c2)oc2ccccc23)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7oc8cccc5c8c67)c5ccccc45)cc3)cccc12 ; c1cc(-c2c3ccccc3c(-c3cc4cccc5oc6cccc3c6c45)c3ccccc23)cc(-c2cccc3c2oc2ccccc23)c1] 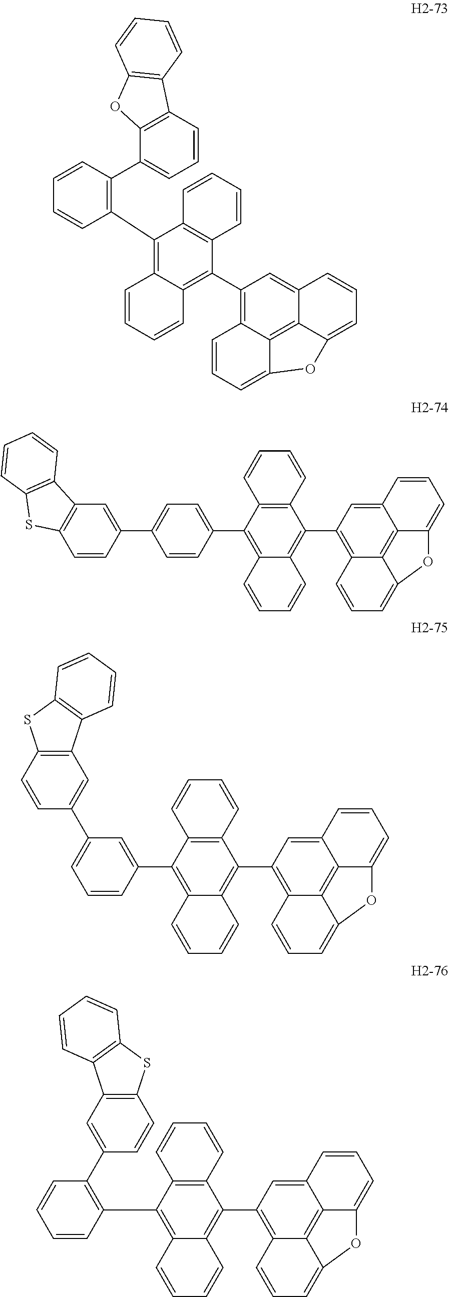 [SMILES: c1ccc(-c2cccc3c2oc2ccccc23)c(-c2c3ccccc3c(-c3cc4cccc5oc6cccc3c6c45)c3ccccc23)c1 ; c1cc2cc(-c3c4ccccc4c(-c4ccc(-c5ccc6sc7ccccc7c6c5)cc4)c4ccccc34)c3cccc4oc(c1)c2c43 ; c1cc(-c2ccc3sc4ccccc4c3c2)cc(-c2c3ccccc3c(-c3cc4cccc5oc6cccc3c6c45)c3ccccc23)c1 ; c1ccc(-c2c3ccccc3c(-c3cc4cccc5oc6cccc3c6c45)c3ccccc23)c(-c2ccc3sc4ccccc4c3c2)c1] 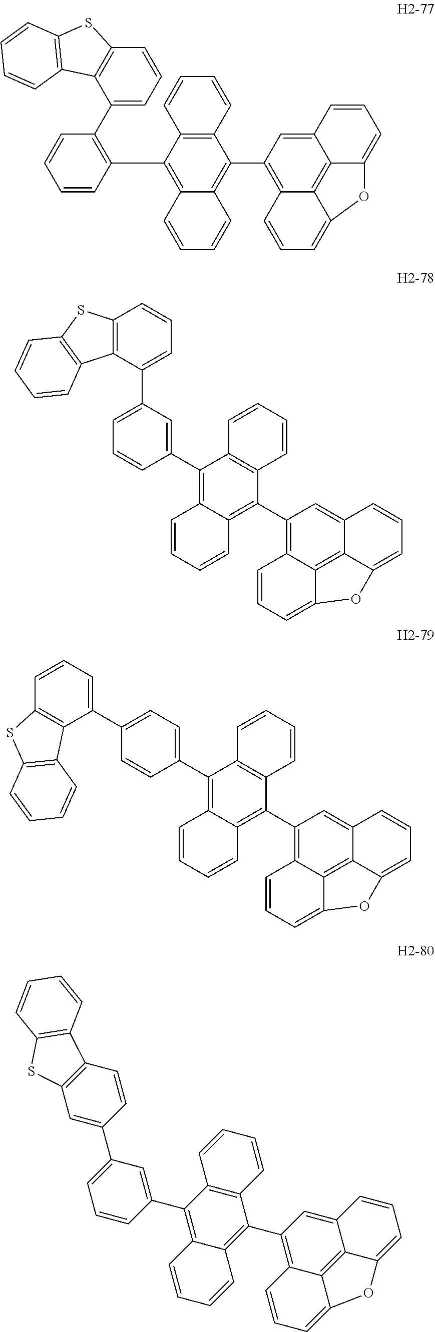 [SMILES: c1ccc(-c2cccc3sc4ccccc4c23)c(-c2c3ccccc3c(-c3cc4cccc5oc6cccc3c6c45)c3ccccc23)c1 ; c1cc(-c2c3ccccc3c(-c3cc4cccc5oc6cccc3c6c45)c3ccccc23)cc(-c2cccc3sc4ccccc4c23)c1 ; c1cc2cc(-c3c4ccccc4c(-c4ccc(-c5cccc6sc7ccccc7c56)cc4)c4ccccc34)c3cccc4oc(c1)c2c43 ; c1cc(-c2ccc3c(c2)sc2ccccc23)cc(-c2c3ccccc3c(-c3cc4cccc5oc6cccc3c6c45)c3ccccc23)c1] 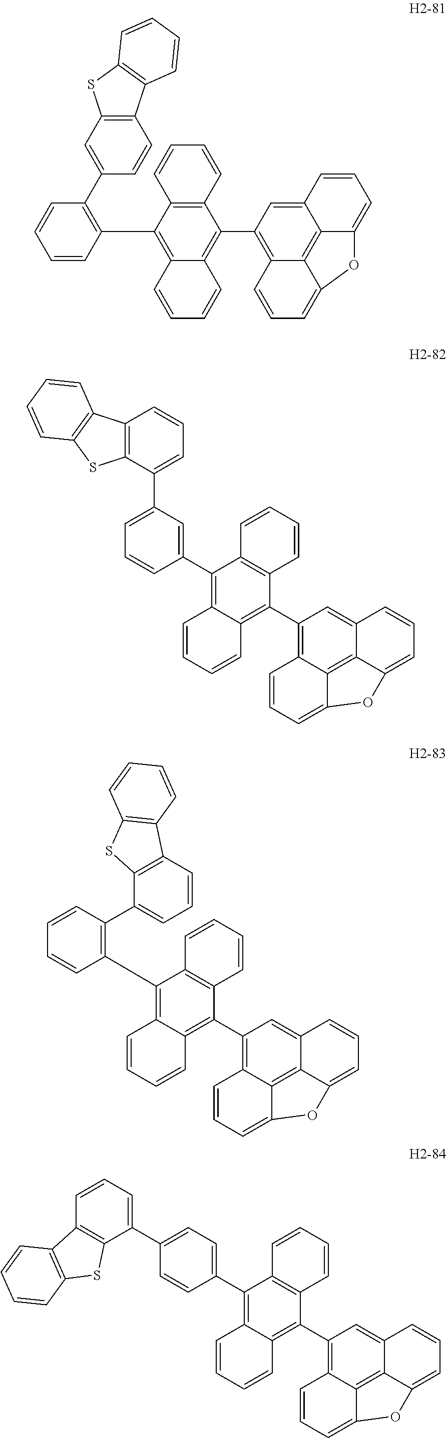 [SMILES: c1ccc(-c2c3ccccc3c(-c3cc4cccc5oc6cccc3c6c45)c3ccccc23)c(-c2ccc3c(c2)sc2ccccc23)c1 ; c1cc(-c2c3ccccc3c(-c3cc4cccc5oc6cccc3c6c45)c3ccccc23)cc(-c2cccc3c2sc2ccccc23)c1 ; c1ccc(-c2cccc3c2sc2ccccc23)c(-c2c3ccccc3c(-c3cc4cccc5oc6cccc3c6c45)c3ccccc23)c1 ; c1cc2cc(-c3c4ccccc4c(-c4ccc(-c5cccc6c5sc5ccccc56)cc4)c4ccccc34)c3cccc4oc(c1)c2c43] 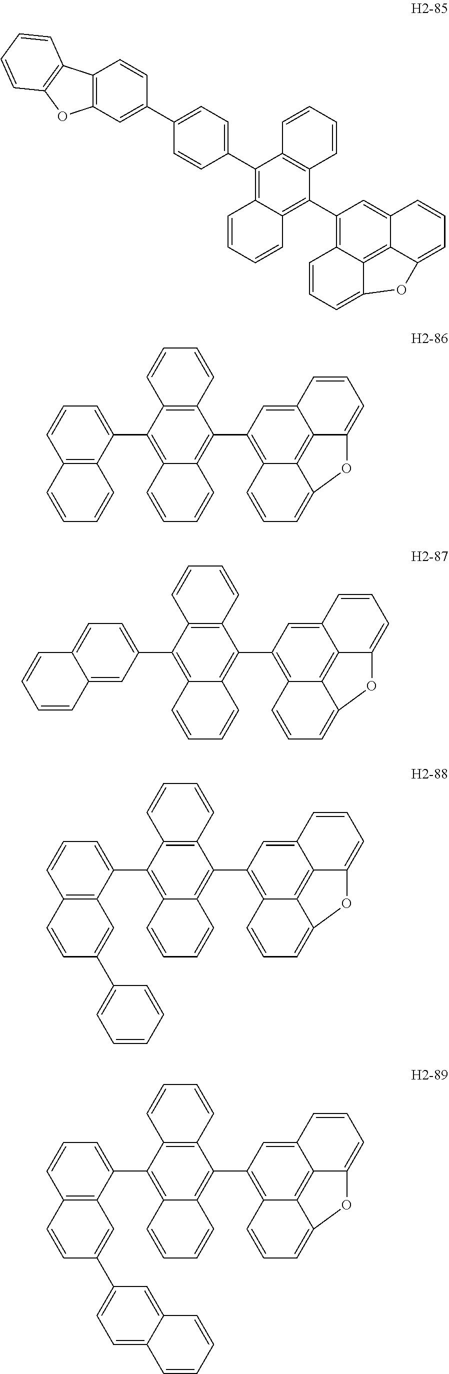 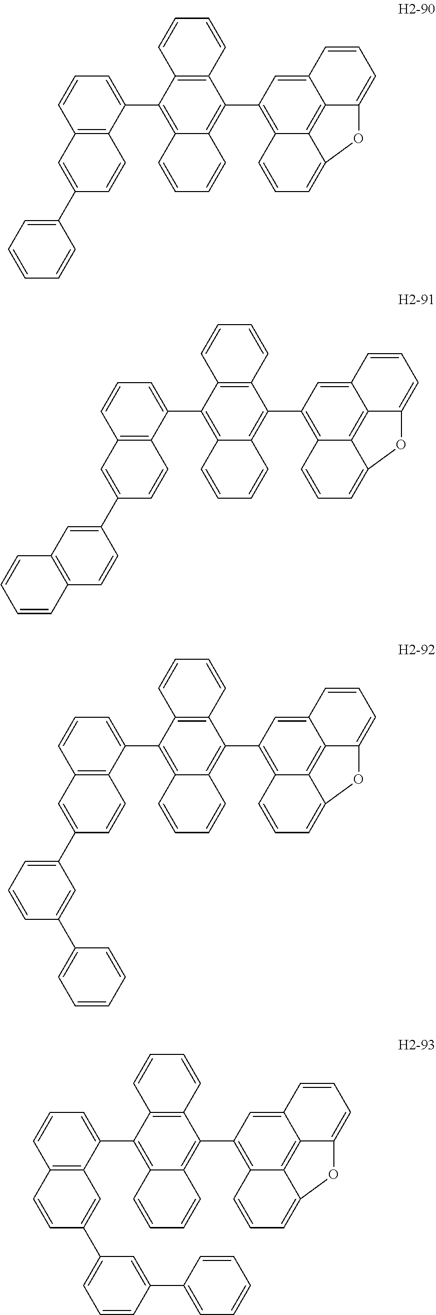 [SMILES: c1ccc(-c2ccc3c(-c4c5ccccc5c(-c5cc6cccc7oc8cccc5c8c67)c5ccccc45)cccc3c2)cc1 ; c1ccc2cc(-c3ccc4c(-c5c6ccccc6c(-c6cc7cccc8oc9cccc6c9c78)c6ccccc56)cccc4c3)ccc2c1 ; c1ccc(-c2cccc(-c3ccc4c(-c5c6ccccc6c(-c6cc7cccc8oc9cccc6c9c78)c6ccccc56)cccc4c3)c2)cc1 ; c1ccc(-c2cccc(-c3ccc4cccc(-c5c6ccccc6c(-c6cc7cccc8oc9cccc6c9c78)c6ccccc56)c4c3)c2)cc1] 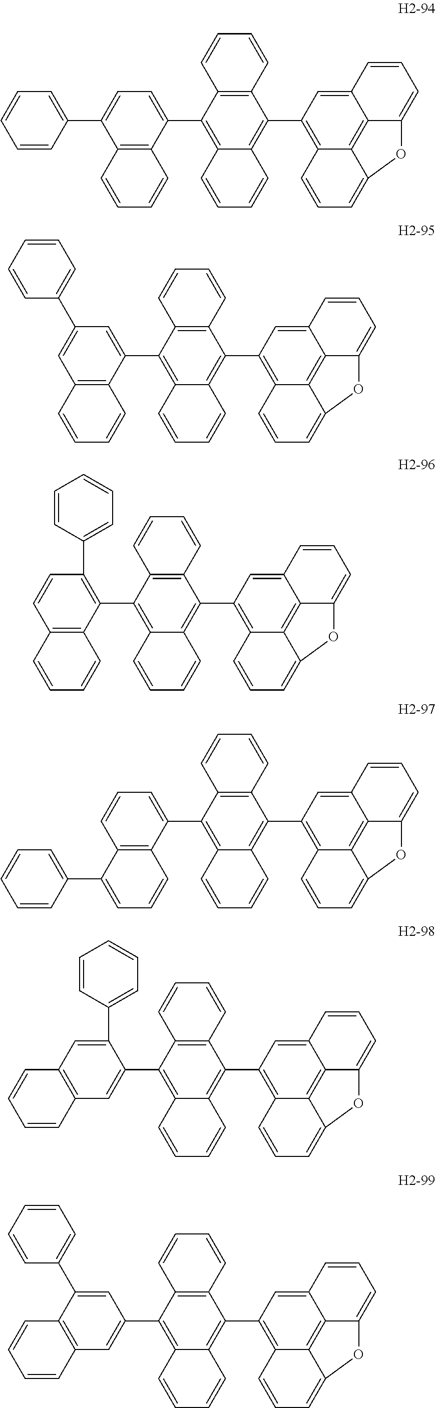 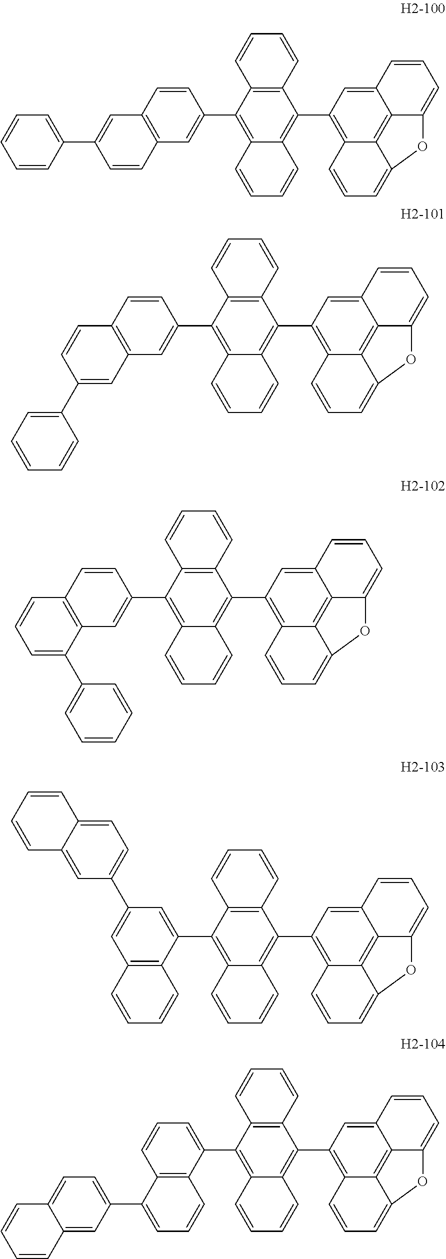 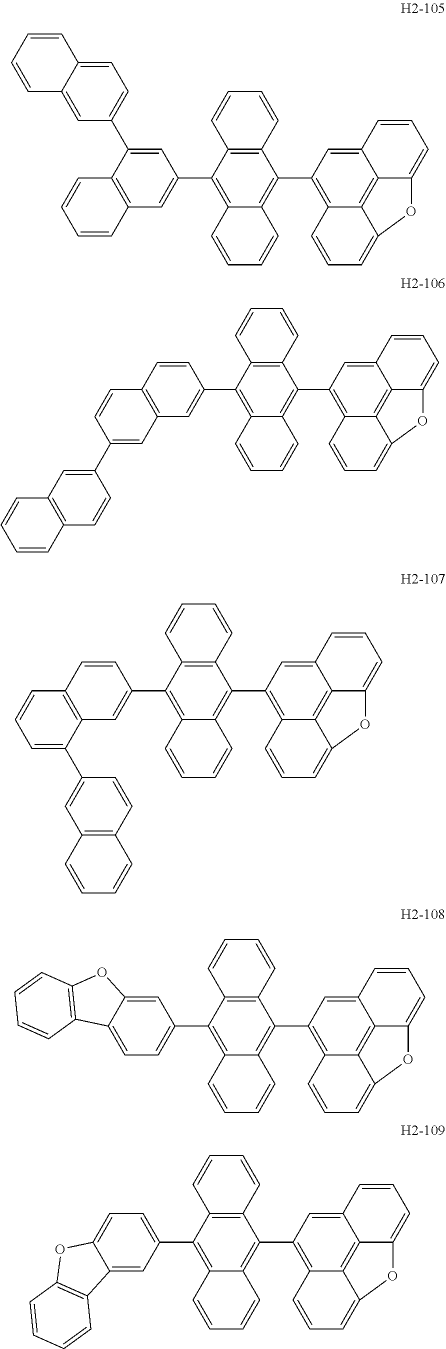 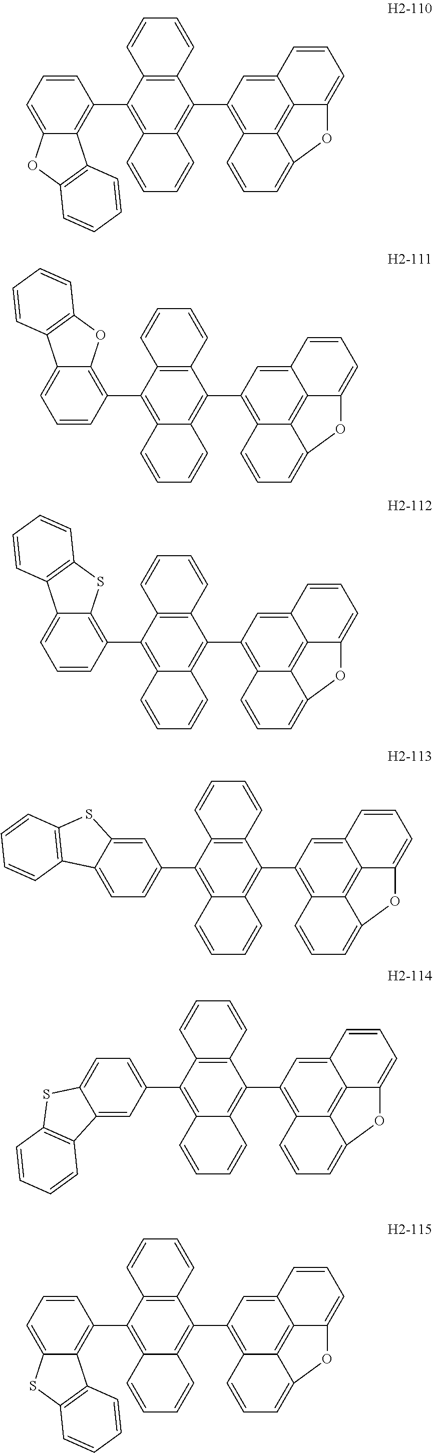 [SMILES: c1ccc2c(c1)oc1cccc(-c3c4ccccc4c(-c4cc5cccc6oc7cccc4c7c56)c4ccccc34)c12 ; c1ccc2c(c1)oc1c(-c3c4ccccc4c(-c4cc5cccc6oc7cccc4c7c56)c4ccccc34)cccc12 ; c1cc2cc(-c3c4ccccc4c(-c4cccc5c4sc4ccccc45)c4ccccc34)c3cccc4oc(c1)c2c43 ; c1cc2cc(-c3c4ccccc4c(-c4ccc5c(c4)sc4ccccc45)c4ccccc34)c3cccc4oc(c1)c2c43 ; c1cc2cc(-c3c4ccccc4c(-c4ccc5sc6ccccc6c5c4)c4ccccc34)c3cccc4oc(c1)c2c43 ; c1cc2cc(-c3c4ccccc4c(-c4cccc5sc6ccccc6c45)c4ccccc34)c3cccc4oc(c1)c2c43] 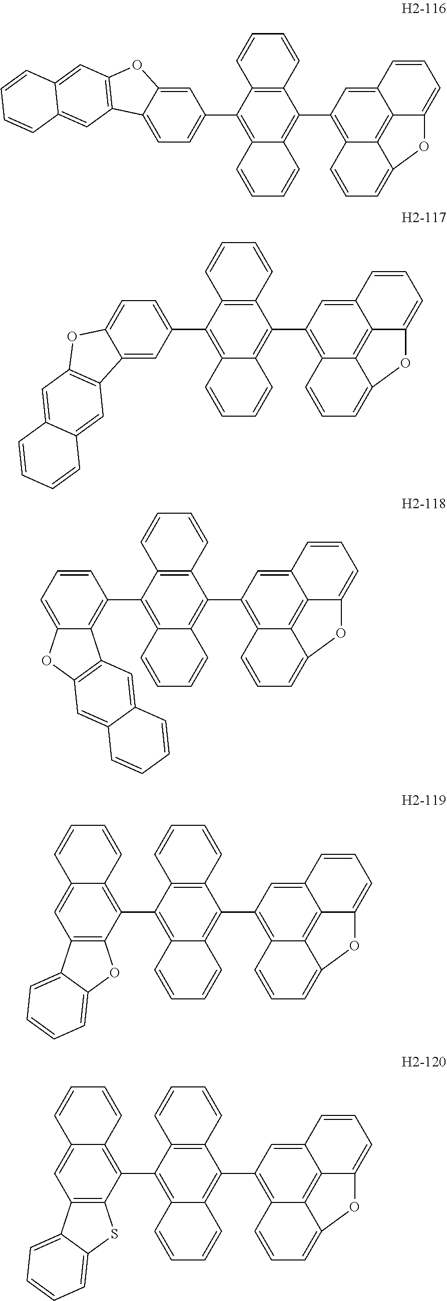 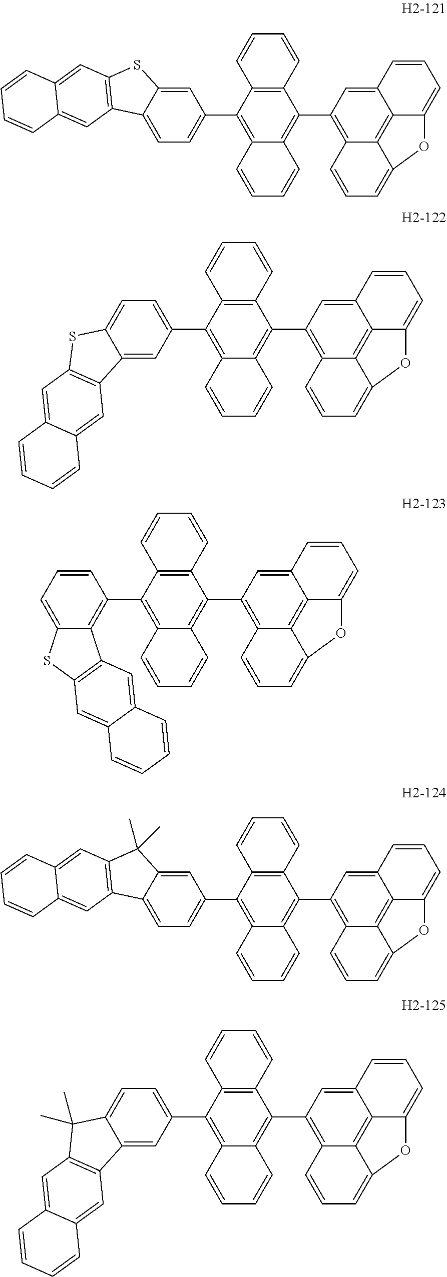 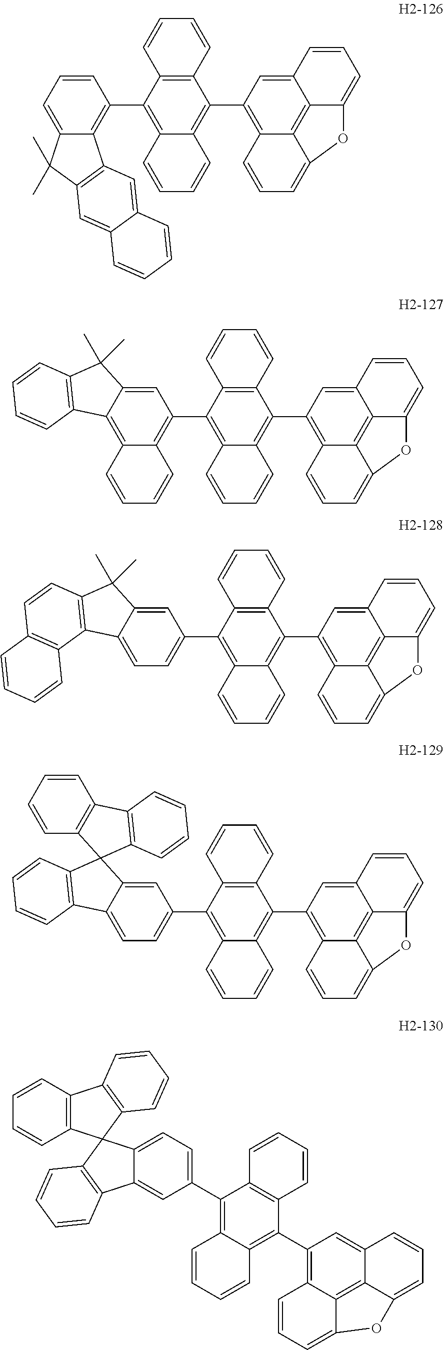 [SMILES: CC1(C)c2cc3ccccc3cc2-c2c(-c3c4ccccc4c(-c4cc5cccc6oc7cccc4c7c56)c4ccccc34)cccc21 ; CC1(C)c2ccccc2-c2c1cc(-c1c3ccccc3c(-c3cc4cccc5oc6cccc3c6c45)c3ccccc13)c1ccccc21 ; CC1(C)c2cc(-c3c4ccccc4c(-c4cc5cccc6oc7cccc4c7c56)c4ccccc34)ccc2-c2c1ccc1ccccc21 ; c1ccc2c(c1)-c1ccccc1C21c2ccccc2-c2ccc(-c3c4ccccc4c(-c4cc5cccc6oc7cccc4c7c56)c4ccccc34)cc21 ; c1ccc2c(c1)-c1ccccc1C21c2ccccc2-c2cc(-c3c4ccccc4c(-c4cc5cccc6oc7cccc4c7c56)c4ccccc34)ccc21] 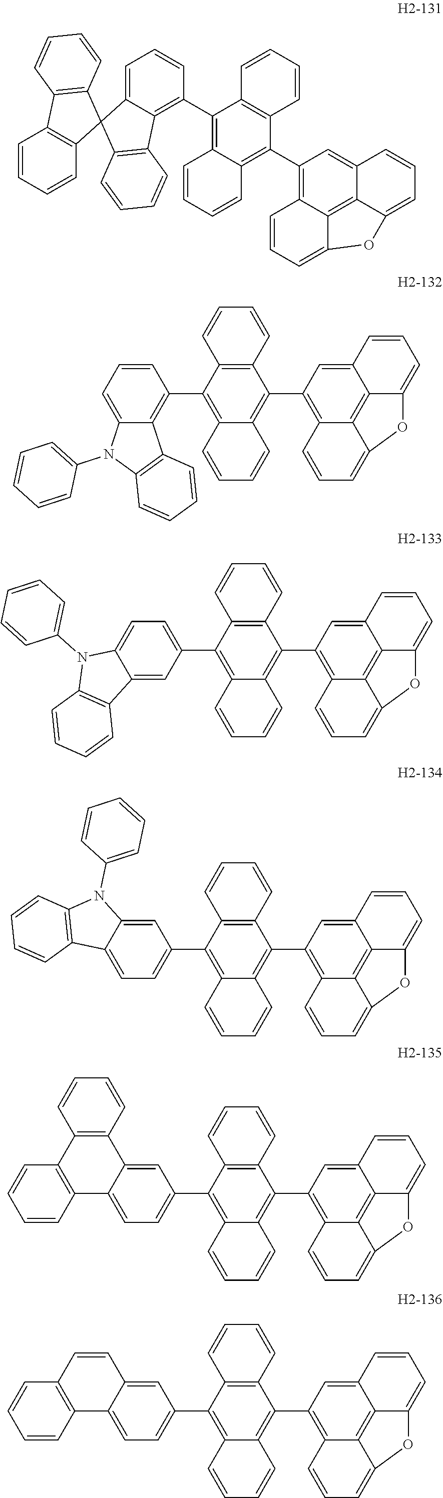 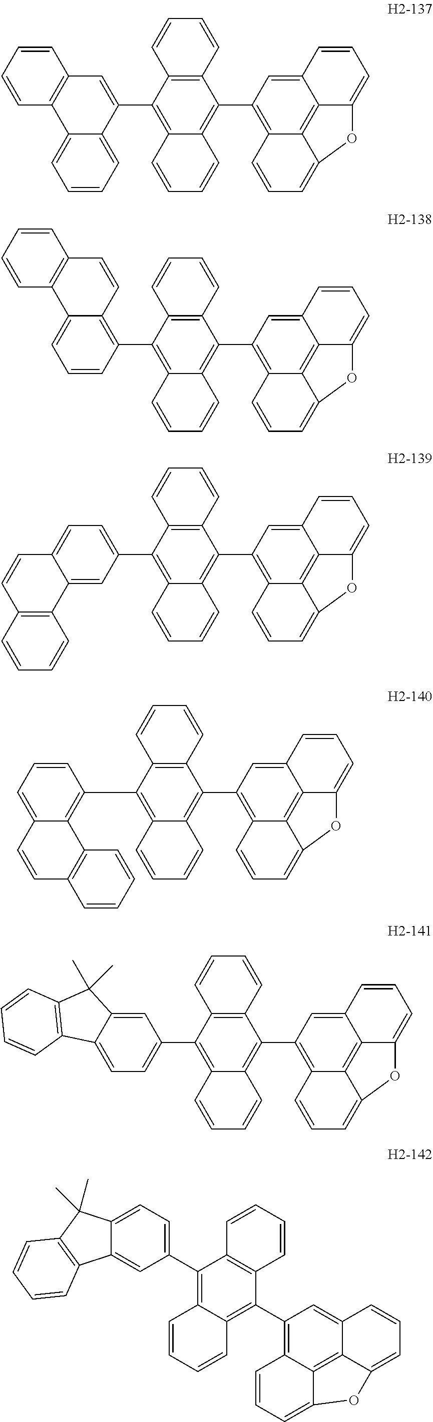 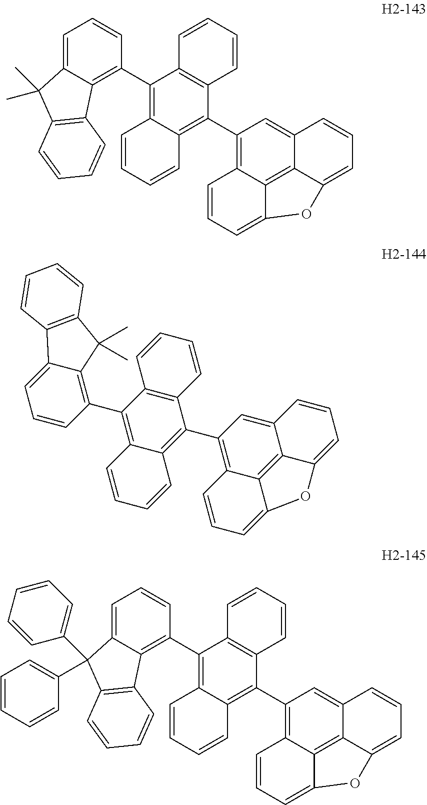 [SMILES: CC1(C)c2ccccc2-c2c(-c3c4ccccc4c(-c4cc5cccc6oc7cccc4c7c56)c4ccccc34)cccc21 ; CC1(C)c2ccccc2-c2cccc(-c3c4ccccc4c(-c4cc5cccc6oc7cccc4c7c56)c4ccccc34)c21 ; c1ccc(C2(c3ccccc3)c3ccccc3-c3c(-c4c5ccccc5c(-c5cc6cccc7oc8cccc5c8c67)c5ccccc45)cccc32)cc1] 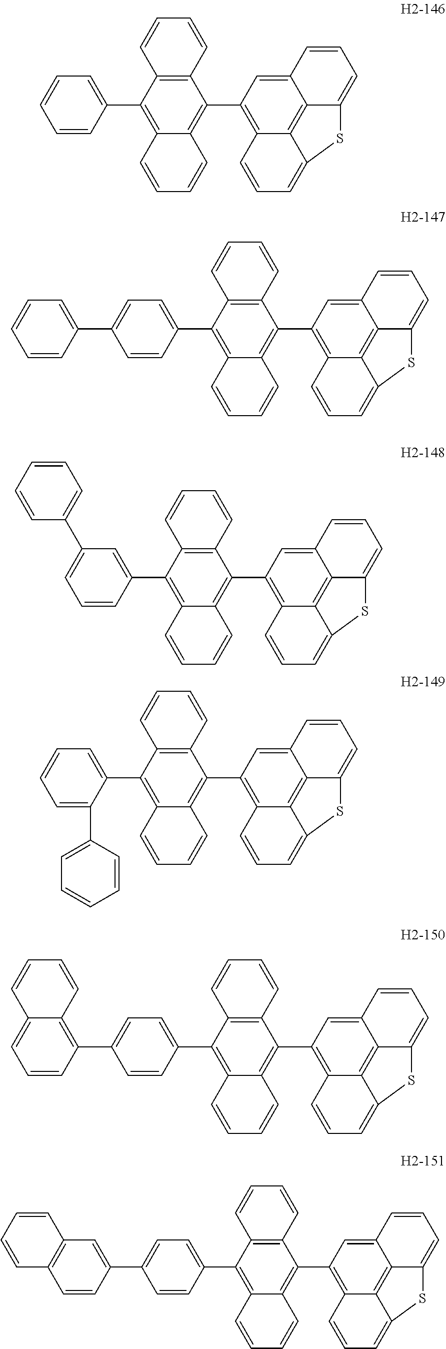 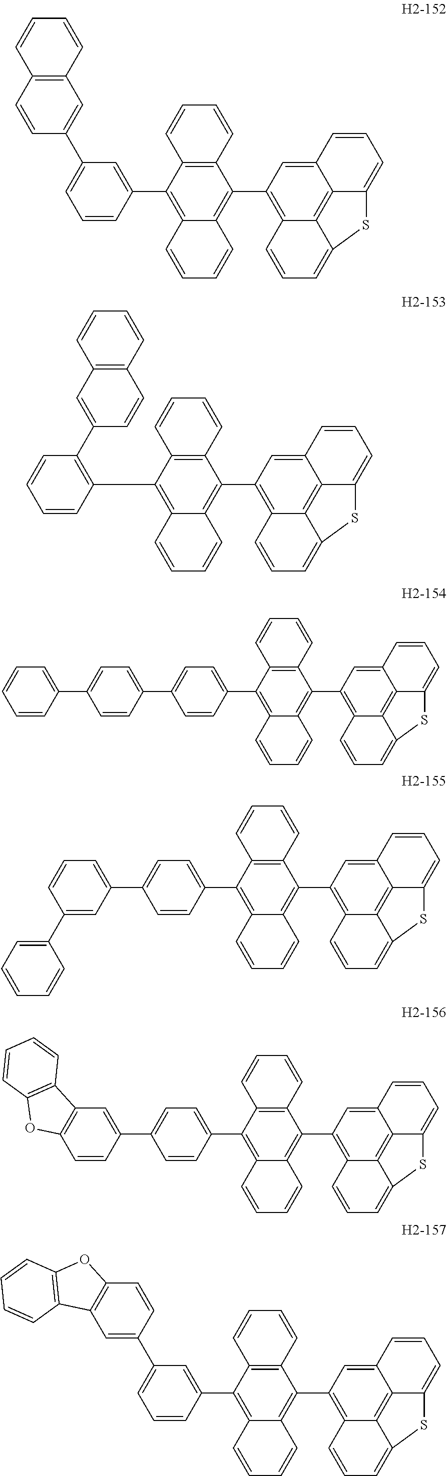 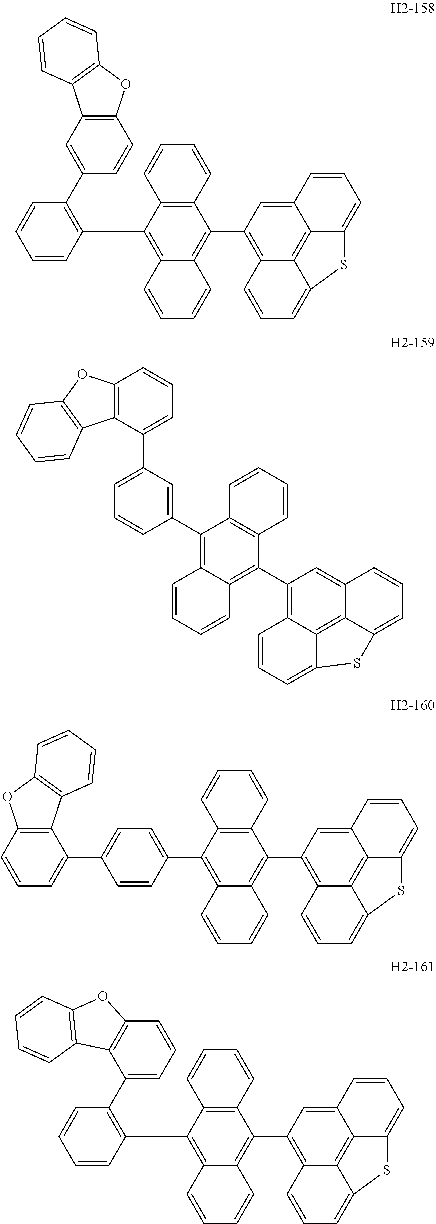 [SMILES: c1ccc(-c2c3ccccc3c(-c3cc4cccc5sc6cccc3c6c45)c3ccccc23)c(-c2ccc3oc4ccccc4c3c2)c1 ; c1cc(-c2c3ccccc3c(-c3cc4cccc5sc6cccc3c6c45)c3ccccc23)cc(-c2cccc3oc4ccccc4c23)c1 ; c1ccc2c(c1)oc1cccc(-c3ccc(-c4c5ccccc5c(-c5cc6cccc7sc8cccc5c8c67)c5ccccc45)cc3)c12 ; c1ccc(-c2cccc3oc4ccccc4c23)c(-c2c3ccccc3c(-c3cc4cccc5sc6cccc3c6c45)c3ccccc23)c1] 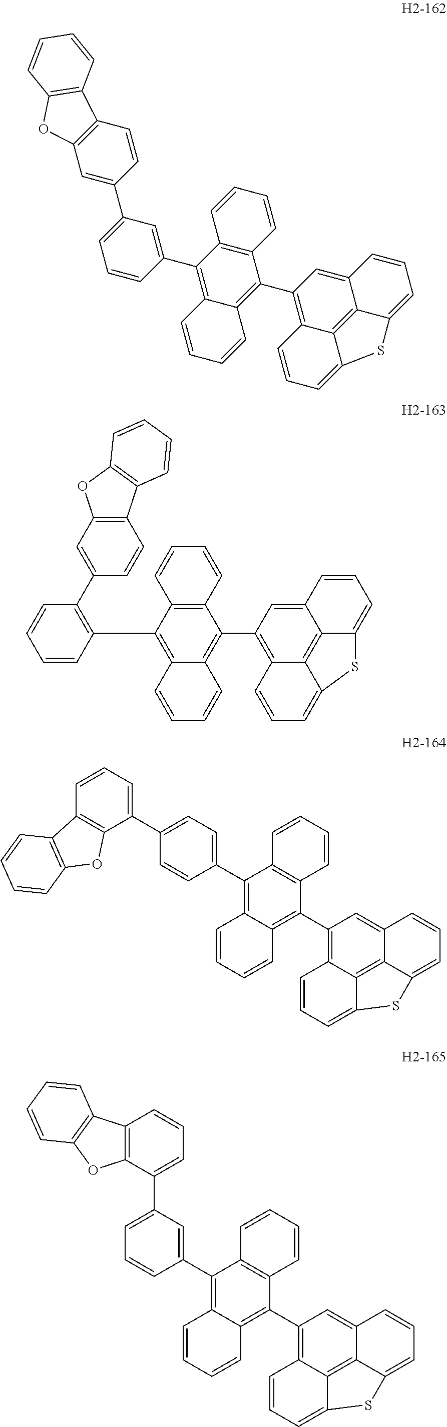 [SMILES: c1cc(-c2ccc3c(c2)oc2ccccc23)cc(-c2c3ccccc3c(-c3cc4cccc5sc6cccc3c6c45)c3ccccc23)c1 ; c1ccc(-c2c3ccccc3c(-c3cc4cccc5sc6cccc3c6c45)c3ccccc23)c(-c2ccc3c(c2)oc2ccccc23)c1 ; c1ccc2c(c1)oc1c(-c3ccc(-c4c5ccccc5c(-c5cc6cccc7sc8cccc5c8c67)c5ccccc45)cc3)cccc12 ; c1cc(-c2c3ccccc3c(-c3cc4cccc5sc6cccc3c6c45)c3ccccc23)cc(-c2cccc3c2oc2ccccc23)c1] 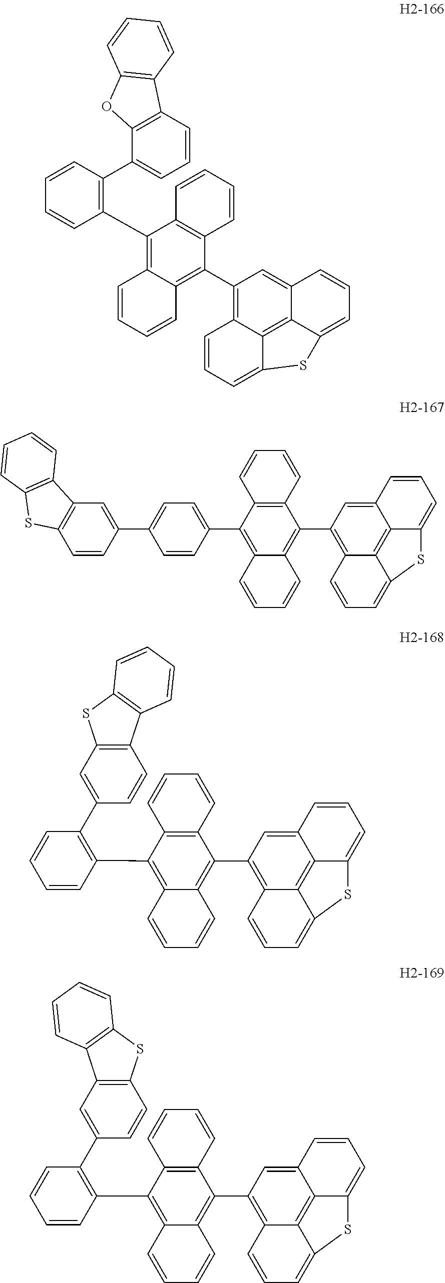 [SMILES: c1ccc(-c2cccc3c2oc2ccccc23)c(-c2c3ccccc3c(-c3cc4cccc5sc6cccc3c6c45)c3ccccc23)c1 ; c1ccc2c(c1)sc1ccc(-c3ccc(-c4c5ccccc5c(-c5cc6cccc7sc8cccc5c8c67)c5ccccc45)cc3)cc12 ; c1ccc(-c2c3ccccc3c(-c3cc4cccc5sc6cccc3c6c45)c3ccccc23)c(-c2ccc3c(c2)sc2ccccc23)c1 ; c1ccc(-c2c3ccccc3c(-c3cc4cccc5sc6cccc3c6c45)c3ccccc23)c(-c2ccc3sc4ccccc4c3c2)c1] 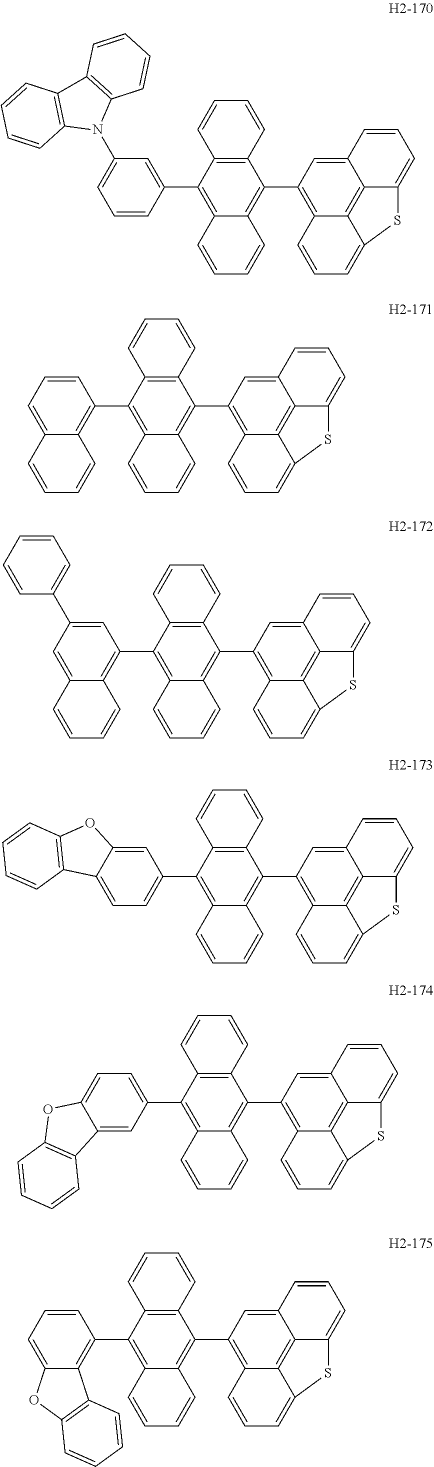 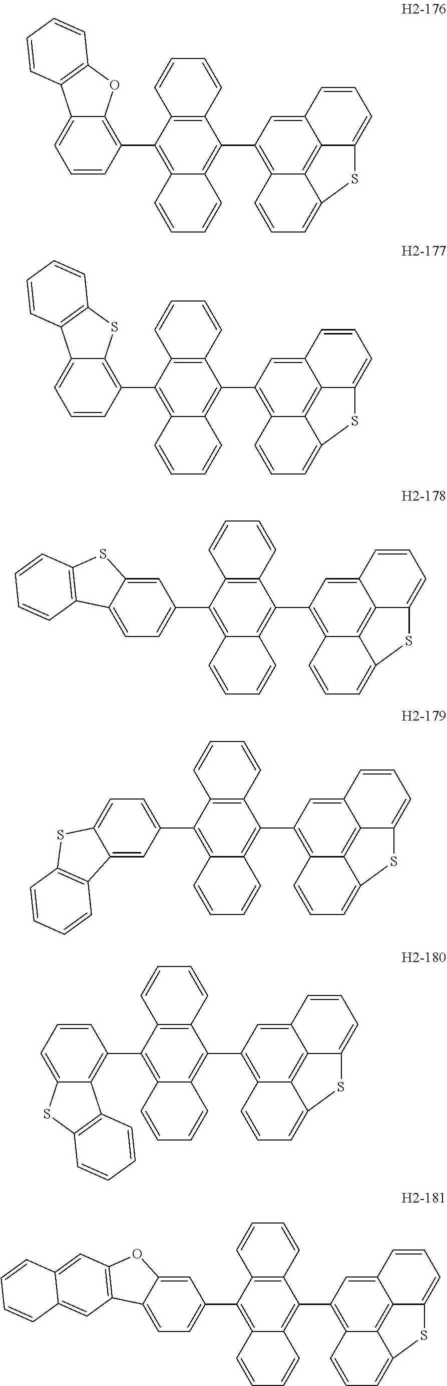 [SMILES: c1ccc2c(c1)oc1c(-c3c4ccccc4c(-c4cc5cccc6sc7cccc4c7c56)c4ccccc34)cccc12 ; c1ccc2c(c1)sc1c(-c3c4ccccc4c(-c4cc5cccc6sc7cccc4c7c56)c4ccccc34)cccc12 ; c1ccc2c(c1)sc1cc(-c3c4ccccc4c(-c4cc5cccc6sc7cccc4c7c56)c4ccccc34)ccc12 ; c1ccc2c(c1)sc1ccc(-c3c4ccccc4c(-c4cc5cccc6sc7cccc4c7c56)c4ccccc34)cc12 ; c1ccc2c(c1)sc1cccc(-c3c4ccccc4c(-c4cc5cccc6sc7cccc4c7c56)c4ccccc34)c12 ; c1ccc2cc3c(cc2c1)oc1cc(-c2c4ccccc4c(-c4cc5cccc6sc7cccc4c7c56)c4ccccc24)ccc13] 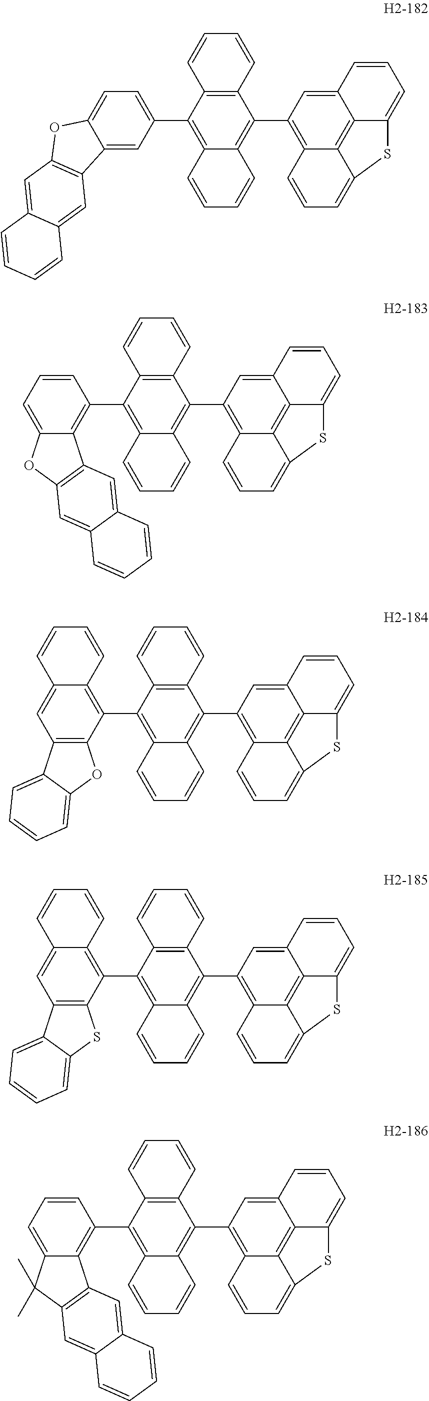 [SMILES: c1ccc2cc3c(cc2c1)oc1ccc(-c2c4ccccc4c(-c4cc5cccc6sc7cccc4c7c56)c4ccccc24)cc13 ; c1ccc2cc3c(cc2c1)oc1cccc(-c2c4ccccc4c(-c4cc5cccc6sc7cccc4c7c56)c4ccccc24)c13 ; c1ccc2c(-c3c4ccccc4c(-c4cc5cccc6sc7cccc4c7c56)c4ccccc34)c3oc4ccccc4c3cc2c1 ; c1ccc2c(-c3c4ccccc4c(-c4cc5cccc6sc7cccc4c7c56)c4ccccc34)c3sc4ccccc4c3cc2c1 ; CC1(C)c2cc3ccccc3cc2-c2c(-c3c4ccccc4c(-c4cc5cccc6sc7cccc4c7c56)c4ccccc34)cccc21] 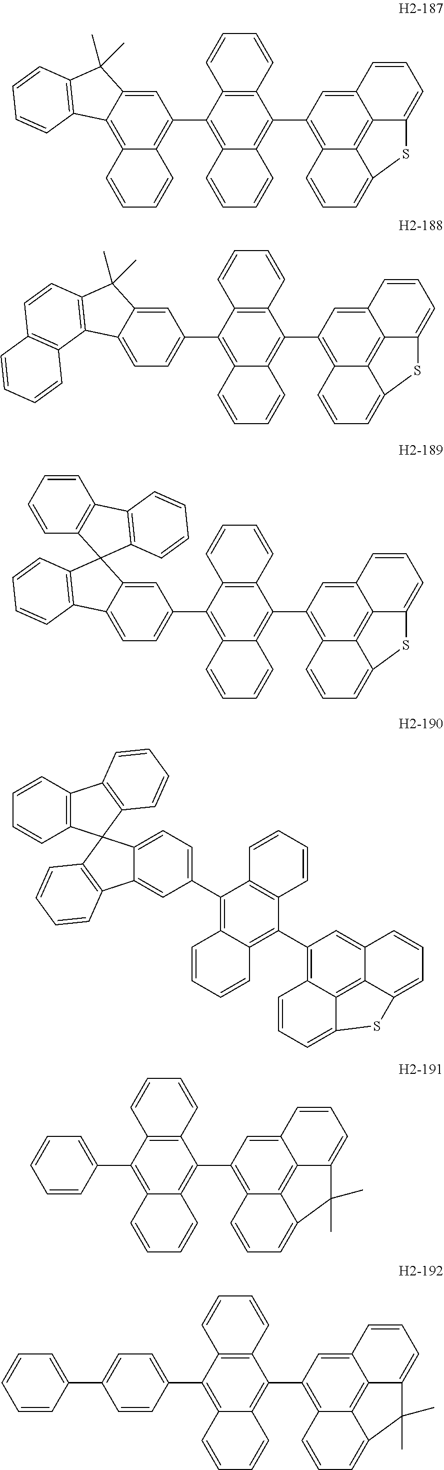 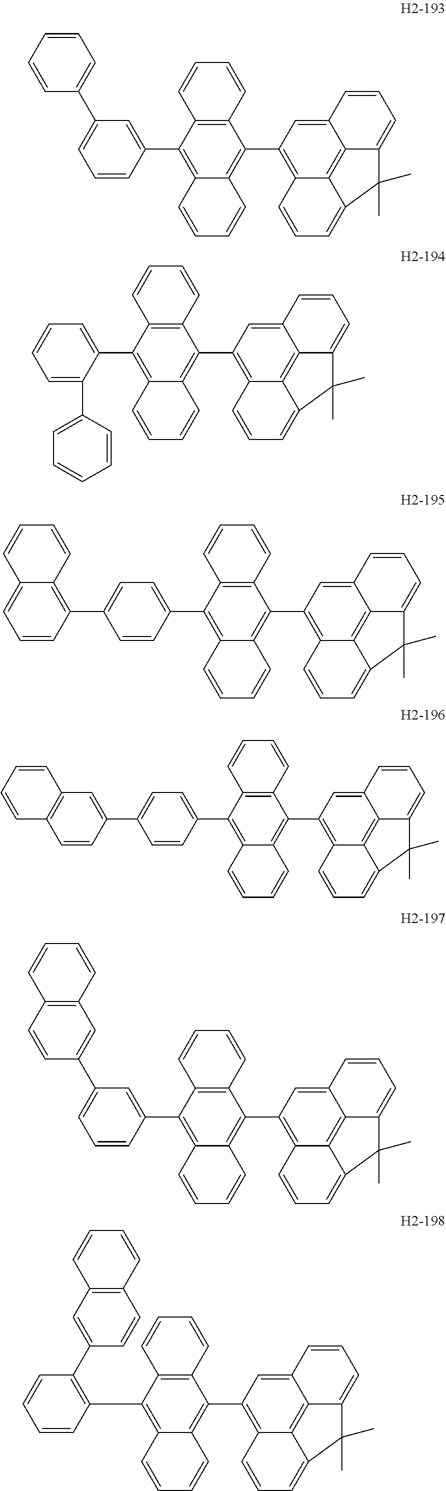 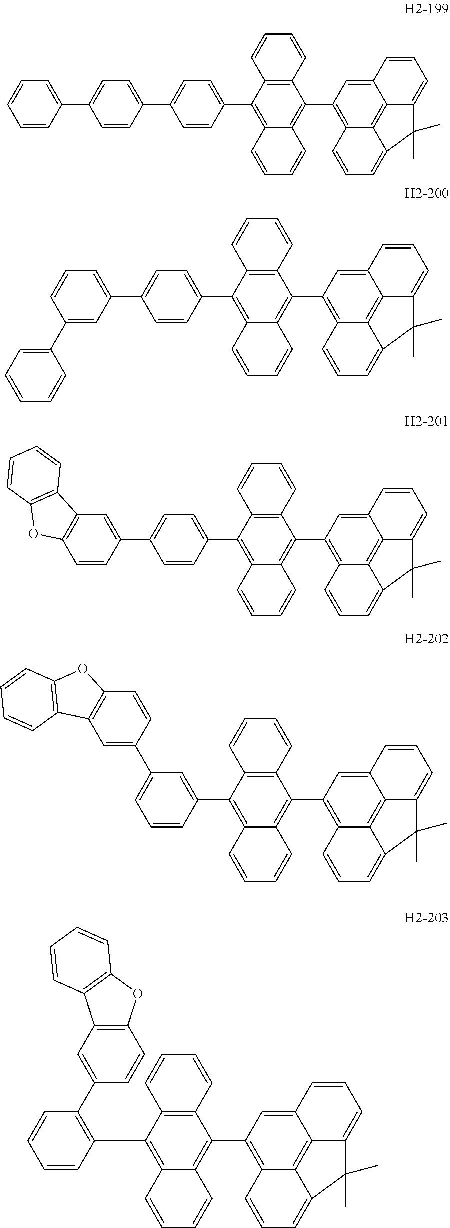 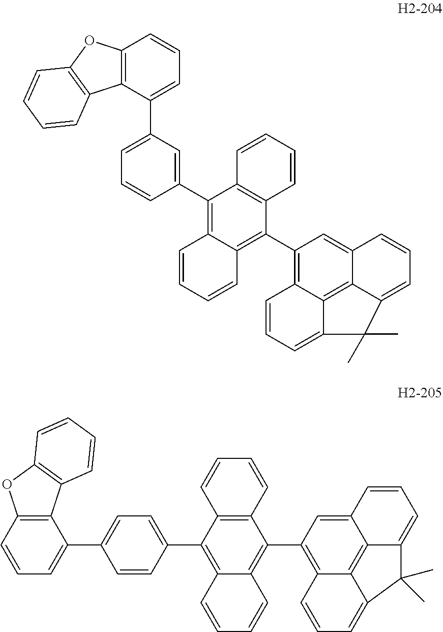 [SMILES: CC1(C)c2cccc3cc(-c4c5ccccc5c(-c5cccc(-c6cccc7oc8ccccc8c67)c5)c5ccccc45)c4cccc1c4c23 ; CC1(C)c2cccc3cc(-c4c5ccccc5c(-c5ccc(-c6cccc7oc8ccccc8c67)cc5)c5ccccc45)c4cccc1c4c23] 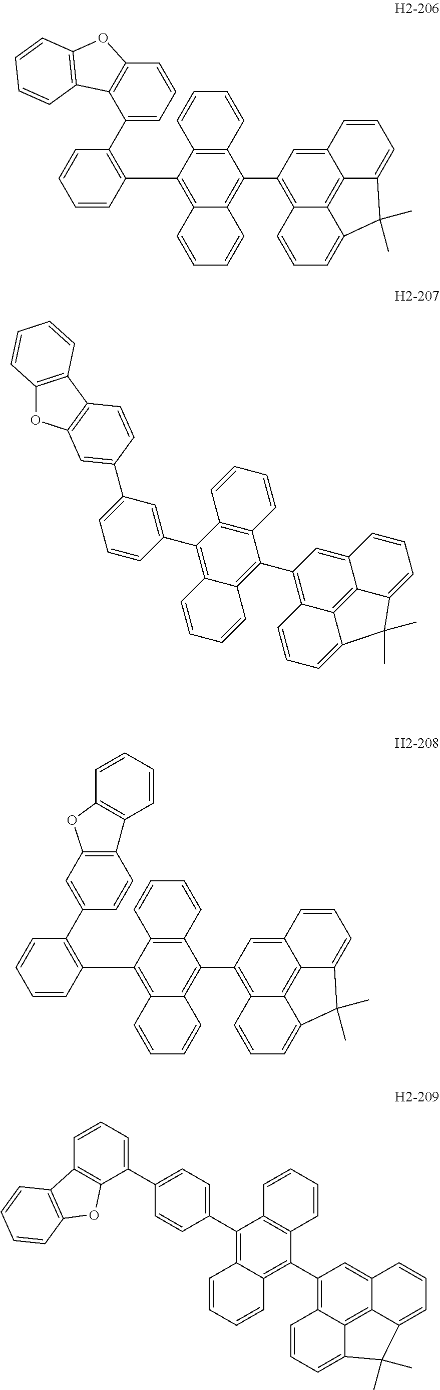 [SMILES: CC1(C)c2cccc3cc(-c4c5ccccc5c(-c5ccccc5-c5cccc6oc7ccccc7c56)c5ccccc45)c4cccc1c4c23 ; CC1(C)c2cccc3cc(-c4c5ccccc5c(-c5cccc(-c6ccc7c(c6)oc6ccccc67)c5)c5ccccc45)c4cccc1c4c23 ; CC1(C)c2cccc3cc(-c4c5ccccc5c(-c5ccccc5-c5ccc6c(c5)oc5ccccc56)c5ccccc45)c4cccc1c4c23 ; CC1(C)c2cccc3cc(-c4c5ccccc5c(-c5ccc(-c6cccc7c6oc6ccccc67)cc5)c5ccccc45)c4cccc1c4c23] 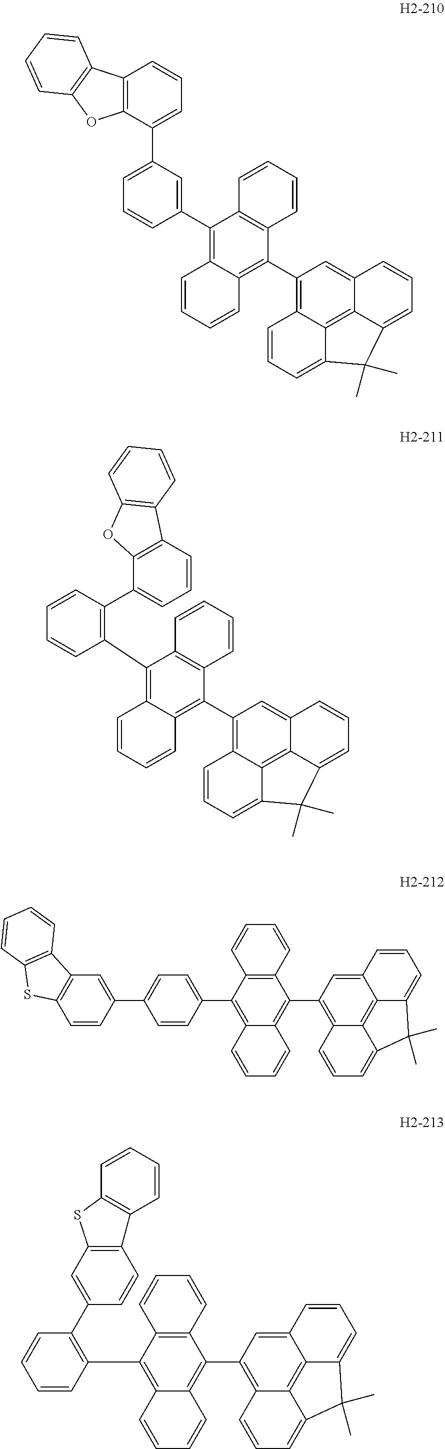 [SMILES: CC1(C)c2cccc3cc(-c4c5ccccc5c(-c5cccc(-c6cccc7c6oc6ccccc67)c5)c5ccccc45)c4cccc1c4c23 ; CC1(C)c2cccc3cc(-c4c5ccccc5c(-c5ccccc5-c5cccc6c5oc5ccccc56)c5ccccc45)c4cccc1c4c23 ; CC1(C)c2cccc3cc(-c4c5ccccc5c(-c5ccc(-c6ccc7sc8ccccc8c7c6)cc5)c5ccccc45)c4cccc1c4c23 ; CC1(C)c2cccc3cc(-c4c5ccccc5c(-c5ccccc5-c5ccc6c(c5)sc5ccccc56)c5ccccc45)c4cccc1c4c23] 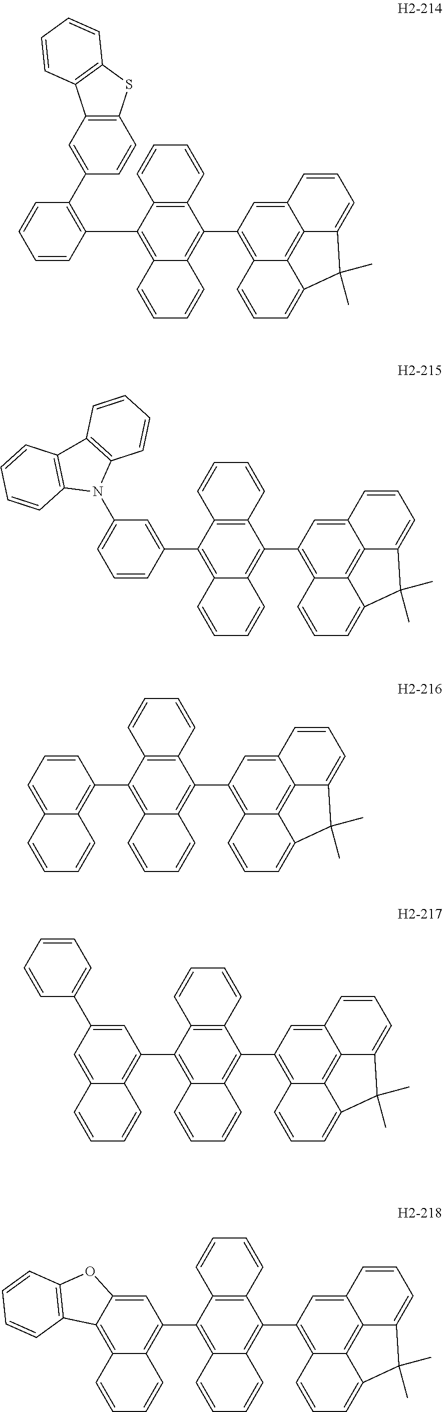 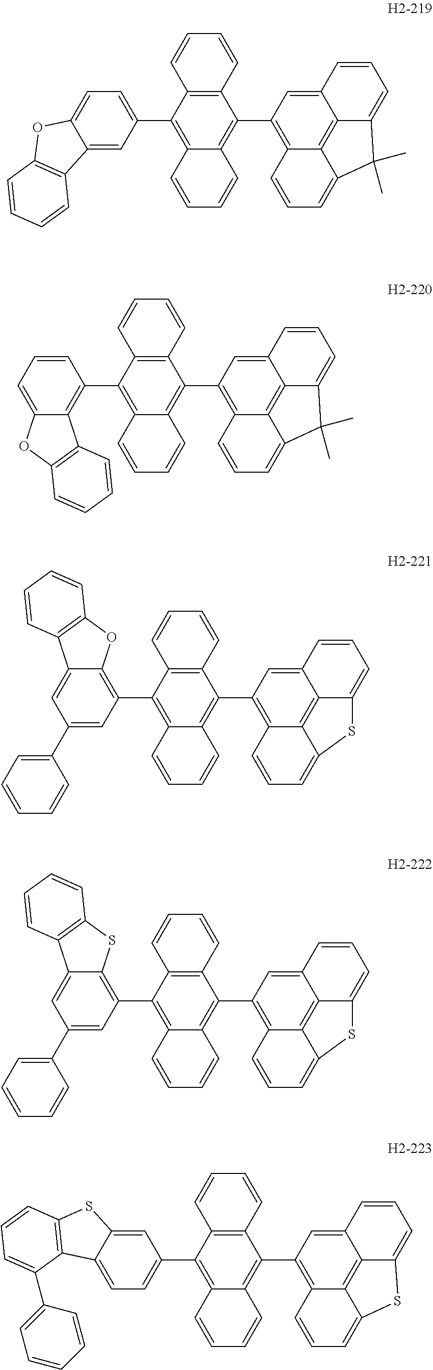 [SMILES: CC1(C)c2cccc3cc(-c4c5ccccc5c(-c5ccc6oc7ccccc7c6c5)c5ccccc45)c4cccc1c4c23 ; CC1(C)c2cccc3cc(-c4c5ccccc5c(-c5cccc6oc7ccccc7c56)c5ccccc45)c4cccc1c4c23 ; c1ccc(-c2cc(-c3c4ccccc4c(-c4cc5cccc6sc7cccc4c7c56)c4ccccc34)c3oc4ccccc4c3c2)cc1 ; c1ccc(-c2cc(-c3c4ccccc4c(-c4cc5cccc6sc7cccc4c7c56)c4ccccc34)c3sc4ccccc4c3c2)cc1 ; c1ccc(-c2cccc3sc4cc(-c5c6ccccc6c(-c6cc7cccc8sc9cccc6c9c78)c6ccccc56)ccc4c23)cc1] 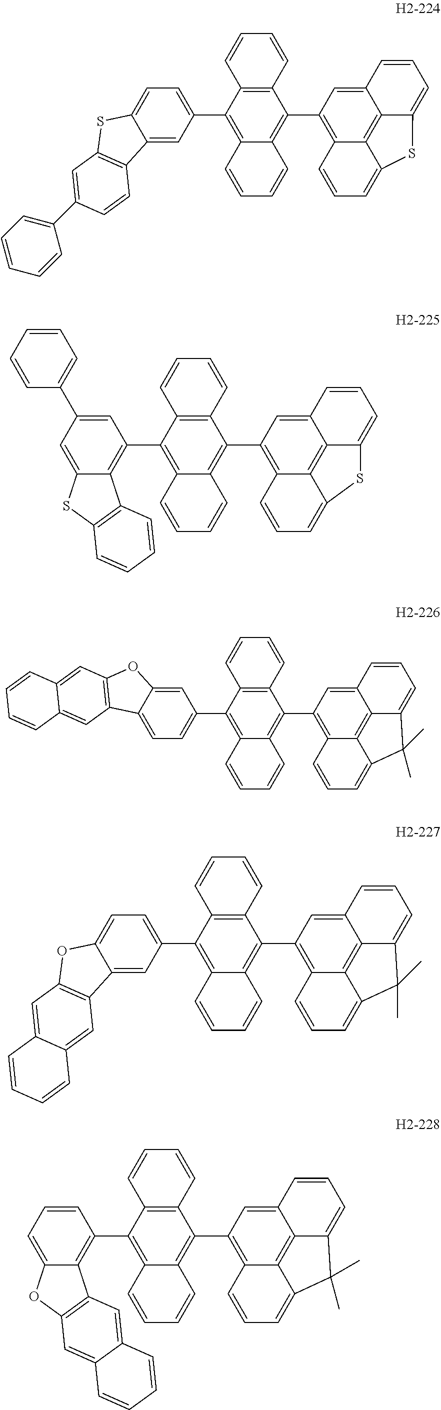 [SMILES: c1ccc(-c2ccc3c(c2)sc2ccc(-c4c5ccccc5c(-c5cc6cccc7sc8cccc5c8c67)c5ccccc45)cc23)cc1 ; c1ccc(-c2cc(-c3c4ccccc4c(-c4cc5cccc6sc7cccc4c7c56)c4ccccc34)c3c(c2)sc2ccccc23)cc1 ; CC1(C)c2cccc3cc(-c4c5ccccc5c(-c5ccc6c(c5)oc5cc7ccccc7cc56)c5ccccc45)c4cccc1c4c23 ; CC1(C)c2cccc3cc(-c4c5ccccc5c(-c5ccc6oc7cc8ccccc8cc7c6c5)c5ccccc45)c4cccc1c4c23 ; CC1(C)c2cccc3cc(-c4c5ccccc5c(-c5cccc6oc7cc8ccccc8cc7c56)c5ccccc45)c4cccc1c4c23] 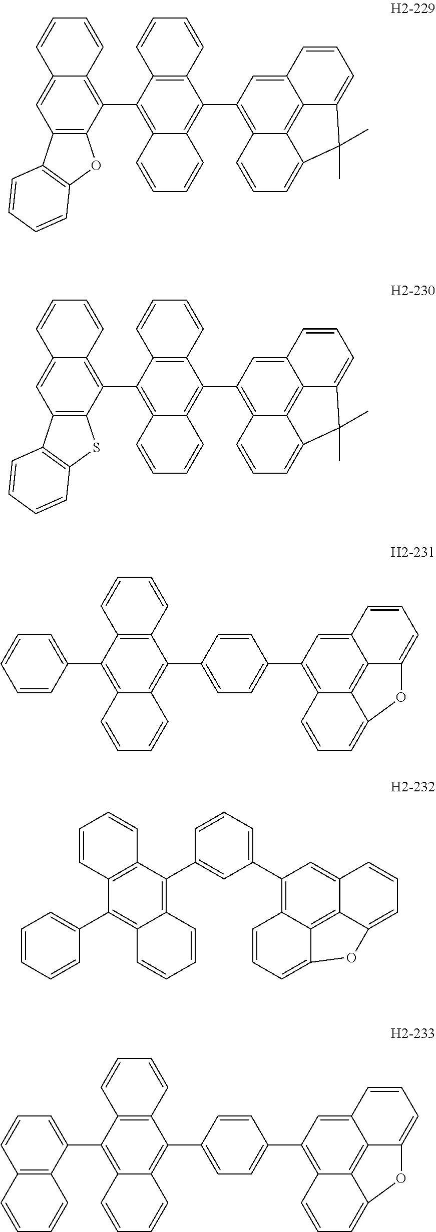 [SMILES: CC1(C)c2cccc3cc(-c4c5ccccc5c(-c5c6ccccc6cc6c5oc5ccccc56)c5ccccc45)c4cccc1c4c23 ; CC1(C)c2cccc3cc(-c4c5ccccc5c(-c5c6ccccc6cc6c5sc5ccccc56)c5ccccc45)c4cccc1c4c23 ; c1ccc(-c2c3ccccc3c(-c3ccc(-c4cc5cccc6oc7cccc4c7c56)cc3)c3ccccc23)cc1 ; c1ccc(-c2c3ccccc3c(-c3cccc(-c4cc5cccc6oc7cccc4c7c56)c3)c3ccccc23)cc1 ; c1ccc2c(-c3c4ccccc4c(-c4ccc(-c5cc6cccc7oc8cccc5c8c67)cc4)c4ccccc34)cccc2c1] 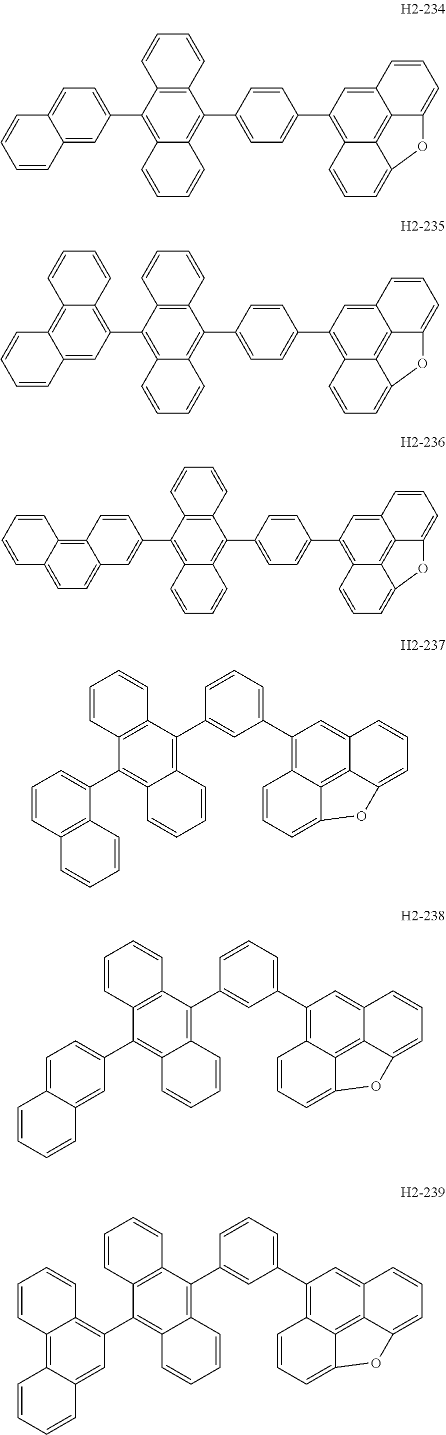 [SMILES: c1ccc2cc(-c3c4ccccc4c(-c4ccc(-c5cc6cccc7oc8cccc5c8c67)cc4)c4ccccc34)ccc2c1 ; c1ccc2c(c1)cc(-c1c3ccccc3c(-c3ccc(-c4cc5cccc6oc7cccc4c7c56)cc3)c3ccccc13)c1ccccc12 ; c1ccc2c(c1)ccc1cc(-c3c4ccccc4c(-c4ccc(-c5cc6cccc7oc8cccc5c8c67)cc4)c4ccccc34)ccc12 ; c1cc(-c2c3ccccc3c(-c3cccc4ccccc34)c3ccccc23)cc(-c2cc3cccc4oc5cccc2c5c34)c1 ; c1cc(-c2c3ccccc3c(-c3ccc4ccccc4c3)c3ccccc23)cc(-c2cc3cccc4oc5cccc2c5c34)c1 ; c1cc(-c2c3ccccc3c(-c3cc4ccccc4c4ccccc34)c3ccccc23)cc(-c2cc3cccc4oc5cccc2c5c34)c1] 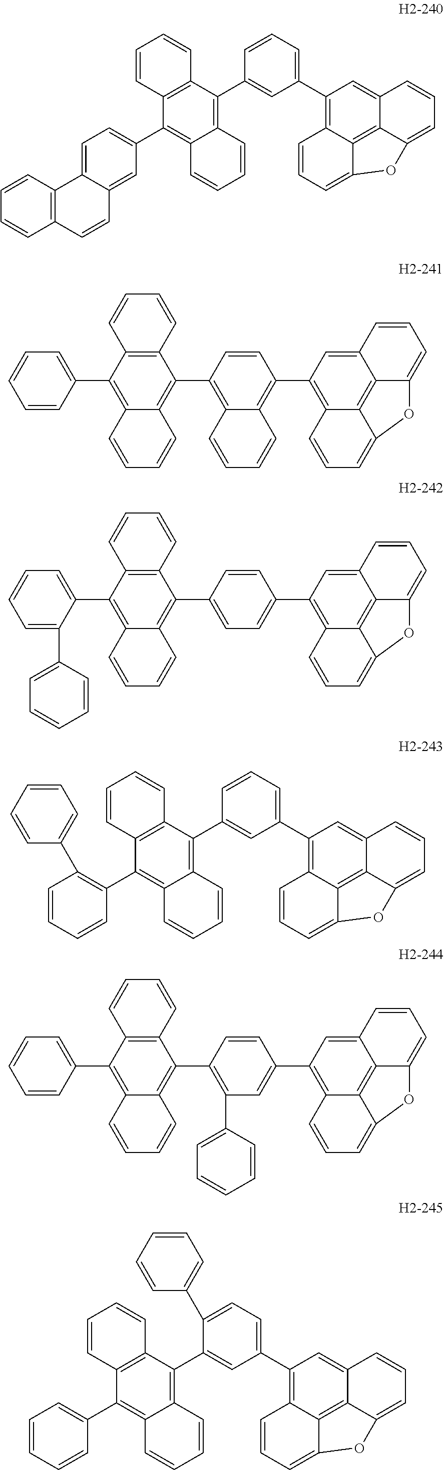 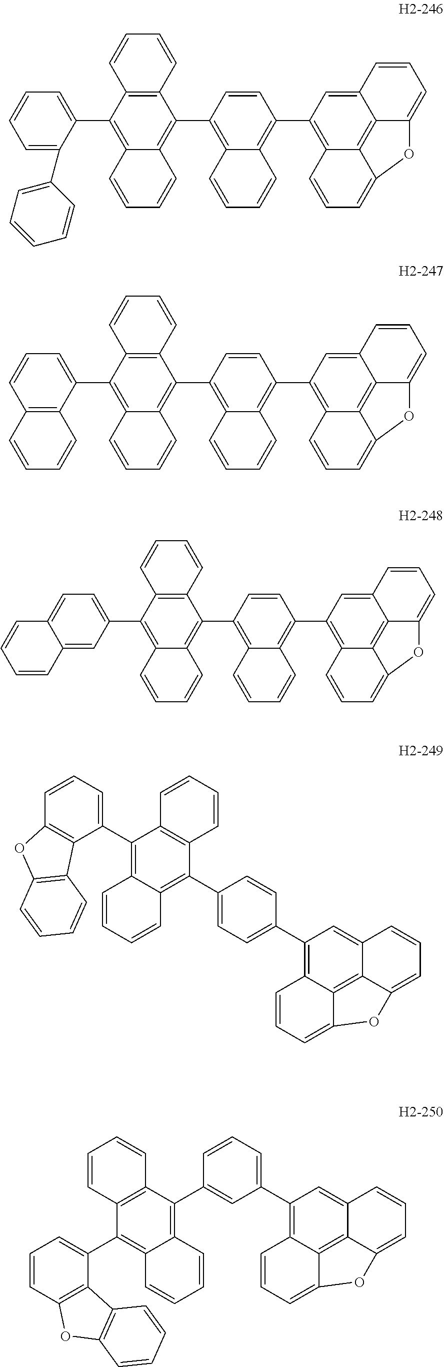 [SMILES: c1ccc(-c2ccccc2-c2c3ccccc3c(-c3ccc(-c4cc5cccc6oc7cccc4c7c56)c4ccccc34)c3ccccc23)cc1 ; c1ccc2c(-c3c4ccccc4c(-c4ccc(-c5cc6cccc7oc8cccc5c8c67)c5ccccc45)c4ccccc34)cccc2c1 ; c1ccc2cc(-c3c4ccccc4c(-c4ccc(-c5cc6cccc7oc8cccc5c8c67)c5ccccc45)c4ccccc34)ccc2c1 ; c1ccc2c(c1)oc1cccc(-c3c4ccccc4c(-c4ccc(-c5cc6cccc7oc8cccc5c8c67)cc4)c4ccccc34)c12 ; c1cc(-c2c3ccccc3c(-c3cccc4oc5ccccc5c34)c3ccccc23)cc(-c2cc3cccc4oc5cccc2c5c34)c1] 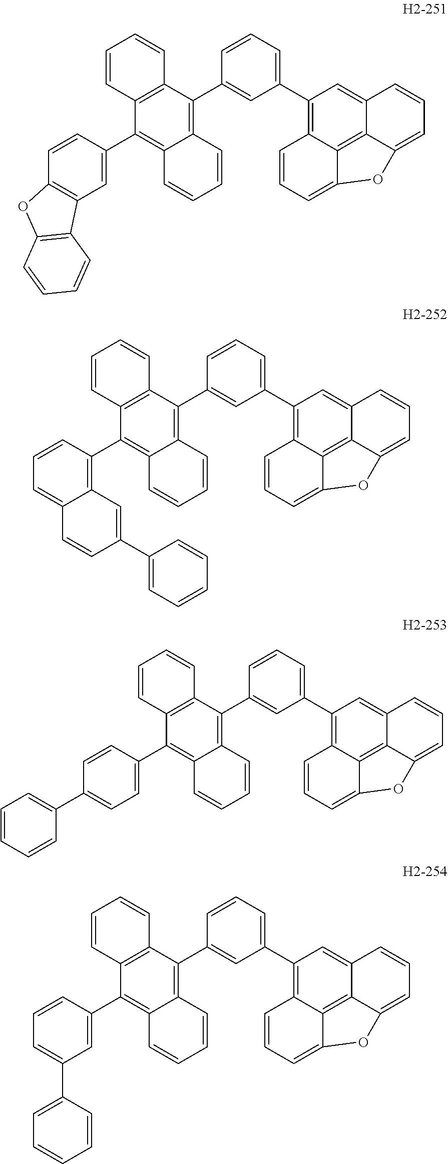 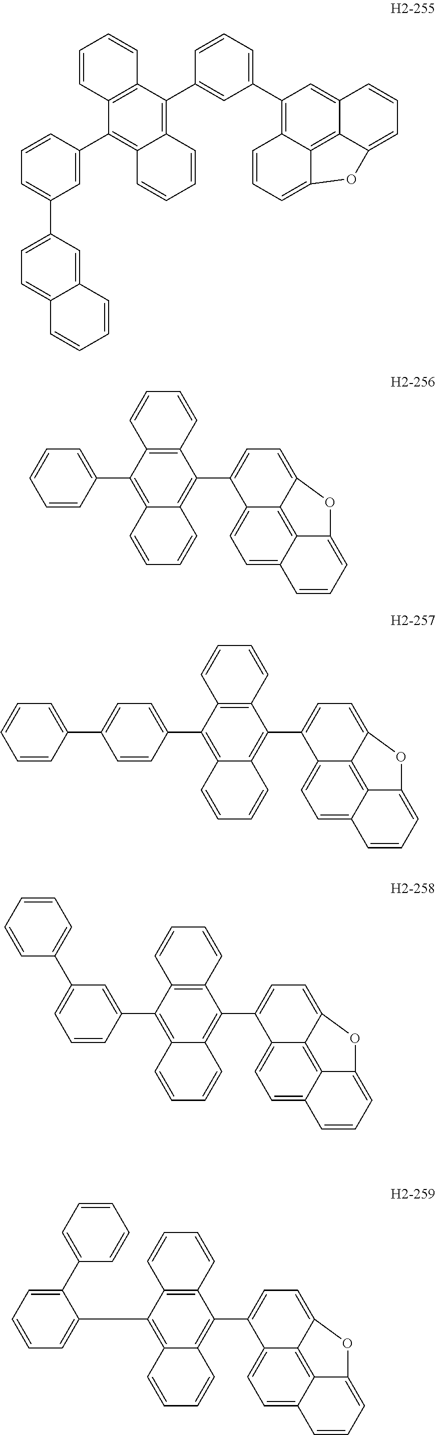 [SMILES: c1cc(-c2ccc3ccccc3c2)cc(-c2c3ccccc3c(-c3cccc(-c4cc5cccc6oc7cccc4c7c56)c3)c3ccccc23)c1 ; c1ccc(-c2c3ccccc3c(-c3ccc4oc5cccc6ccc3c4c65)c3ccccc23)cc1 ; c1ccc(-c2ccc(-c3c4ccccc4c(-c4ccc5oc6cccc7ccc4c5c76)c4ccccc34)cc2)cc1 ; c1ccc(-c2cccc(-c3c4ccccc4c(-c4ccc5oc6cccc7ccc4c5c76)c4ccccc34)c2)cc1 ; c1ccc(-c2ccccc2-c2c3ccccc3c(-c3ccc4oc5cccc6ccc3c4c65)c3ccccc23)cc1] 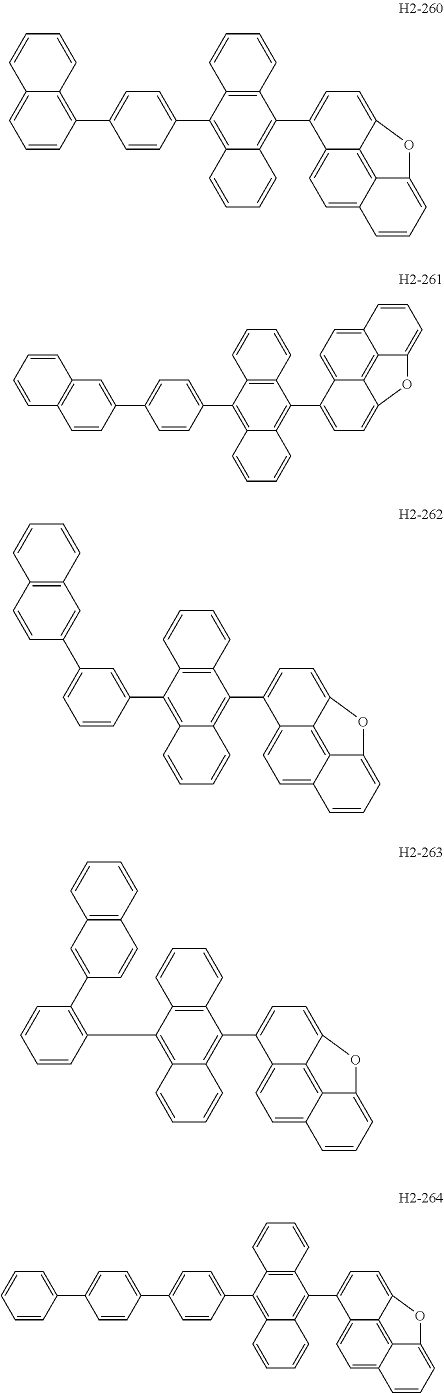 [SMILES: c1ccc2c(-c3ccc(-c4c5ccccc5c(-c5ccc6oc7cccc8ccc5c6c87)c5ccccc45)cc3)cccc2c1 ; c1ccc2cc(-c3ccc(-c4c5ccccc5c(-c5ccc6oc7cccc8ccc5c6c87)c5ccccc45)cc3)ccc2c1 ; c1cc(-c2ccc3ccccc3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3ccccc3c2)c1 ; c1ccc(-c2ccc(-c3ccc(-c4c5ccccc5c(-c5ccc6oc7cccc8ccc5c6c87)c5ccccc45)cc3)cc2)cc1] 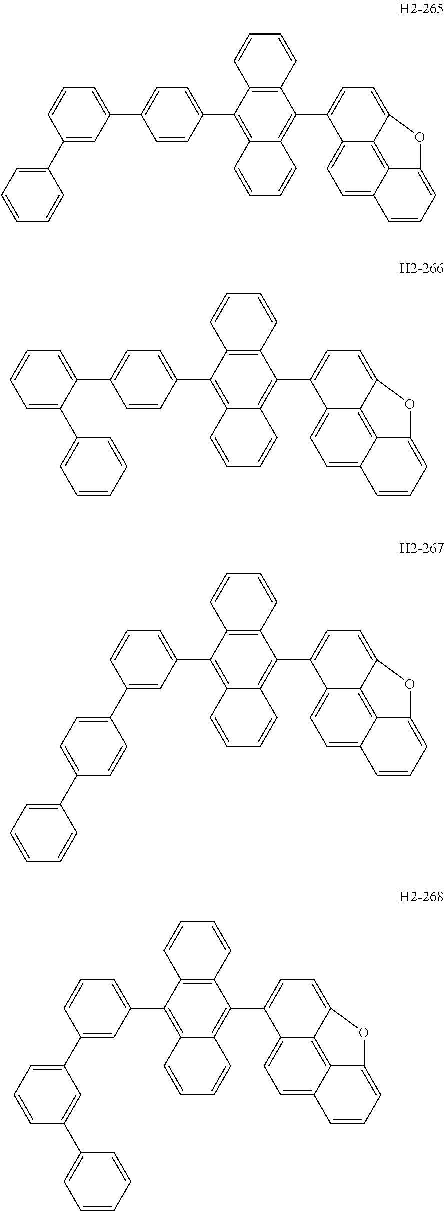 [SMILES: c1ccc(-c2cccc(-c3ccc(-c4c5ccccc5c(-c5ccc6oc7cccc8ccc5c6c87)c5ccccc45)cc3)c2)cc1 ; c1ccc(-c2ccccc2-c2ccc(-c3c4ccccc4c(-c4ccc5oc6cccc7ccc4c5c76)c4ccccc34)cc2)cc1 ; c1ccc(-c2ccc(-c3cccc(-c4c5ccccc5c(-c5ccc6oc7cccc8ccc5c6c87)c5ccccc45)c3)cc2)cc1 ; c1ccc(-c2cccc(-c3cccc(-c4c5ccccc5c(-c5ccc6oc7cccc8ccc5c6c87)c5ccccc45)c3)c2)cc1] 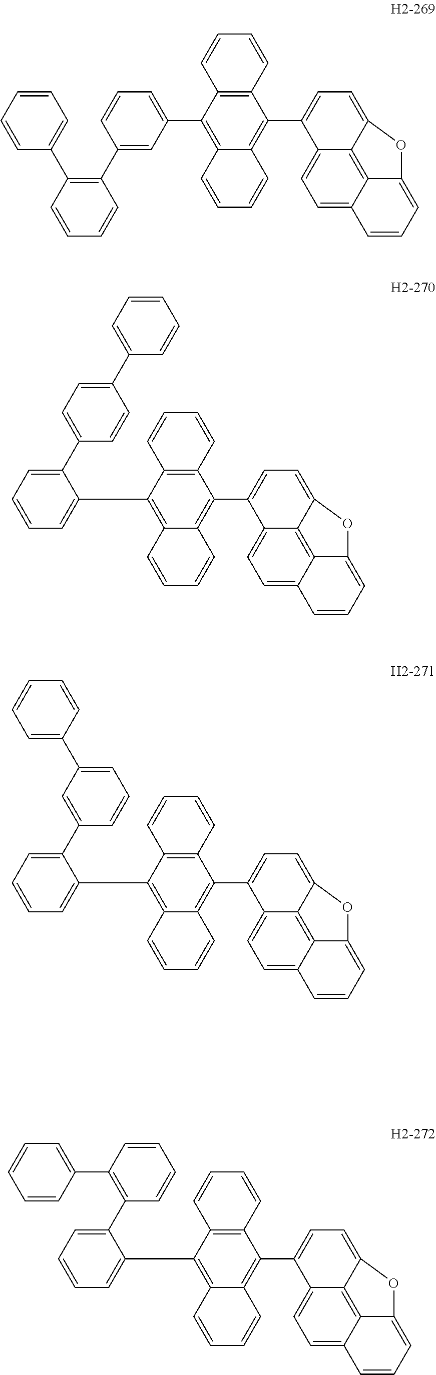 [SMILES: c1ccc(-c2ccccc2-c2cccc(-c3c4ccccc4c(-c4ccc5oc6cccc7ccc4c5c76)c4ccccc34)c2)cc1 ; c1ccc(-c2ccc(-c3ccccc3-c3c4ccccc4c(-c4ccc5oc6cccc7ccc4c5c76)c4ccccc34)cc2)cc1 ; c1ccc(-c2cccc(-c3ccccc3-c3c4ccccc4c(-c4ccc5oc6cccc7ccc4c5c76)c4ccccc34)c2)cc1 ; c1ccc(-c2ccccc2-c2ccccc2-c2c3ccccc3c(-c3ccc4oc5cccc6ccc3c4c65)c3ccccc23)cc1] 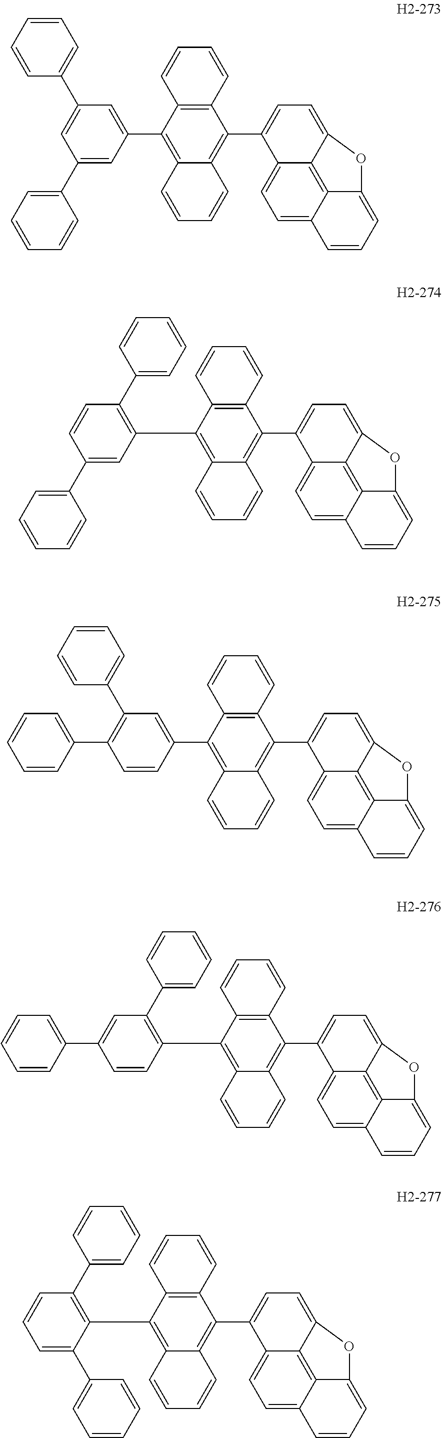 [SMILES: c1ccc(-c2cc(-c3ccccc3)cc(-c3c4ccccc4c(-c4ccc5oc6cccc7ccc4c5c76)c4ccccc34)c2)cc1 ; c1ccc(-c2ccc(-c3ccccc3)c(-c3c4ccccc4c(-c4ccc5oc6cccc7ccc4c5c76)c4ccccc34)c2)cc1 ; c1ccc(-c2ccc(-c3c4ccccc4c(-c4ccc5oc6cccc7ccc4c5c76)c4ccccc34)cc2-c2ccccc2)cc1 ; c1ccc(-c2ccc(-c3c4ccccc4c(-c4ccc5oc6cccc7ccc4c5c76)c4ccccc34)c(-c3ccccc3)c2)cc1 ; c1ccc(-c2cccc(-c3ccccc3)c2-c2c3ccccc3c(-c3ccc4oc5cccc6ccc3c4c65)c3ccccc23)cc1] 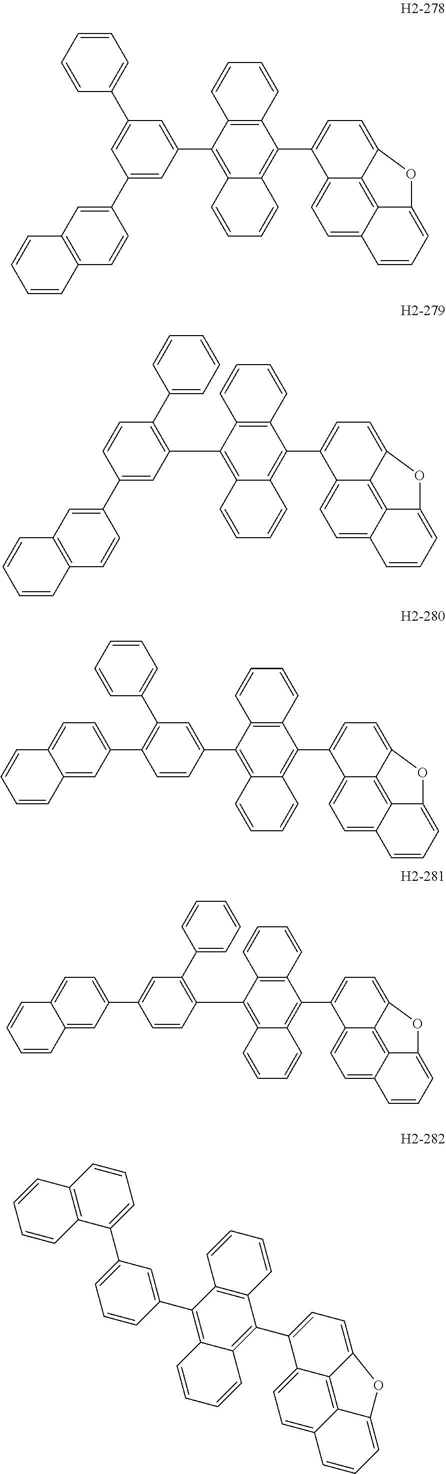 [SMILES: c1ccc(-c2cc(-c3ccc4ccccc4c3)cc(-c3c4ccccc4c(-c4ccc5oc6cccc7ccc4c5c76)c4ccccc34)c2)cc1 ; c1ccc(-c2ccc(-c3ccc4ccccc4c3)cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)ccc2-c2ccc3ccccc3c2)cc1 ; c1ccc(-c2cc(-c3ccc4ccccc4c3)ccc2-c2c3ccccc3c(-c3ccc4oc5cccc6ccc3c4c65)c3ccccc23)cc1 ; c1cc(-c2cccc3ccccc23)cc(-c2c3ccccc3c(-c3ccc4oc5cccc6ccc3c4c65)c3ccccc23)c1] 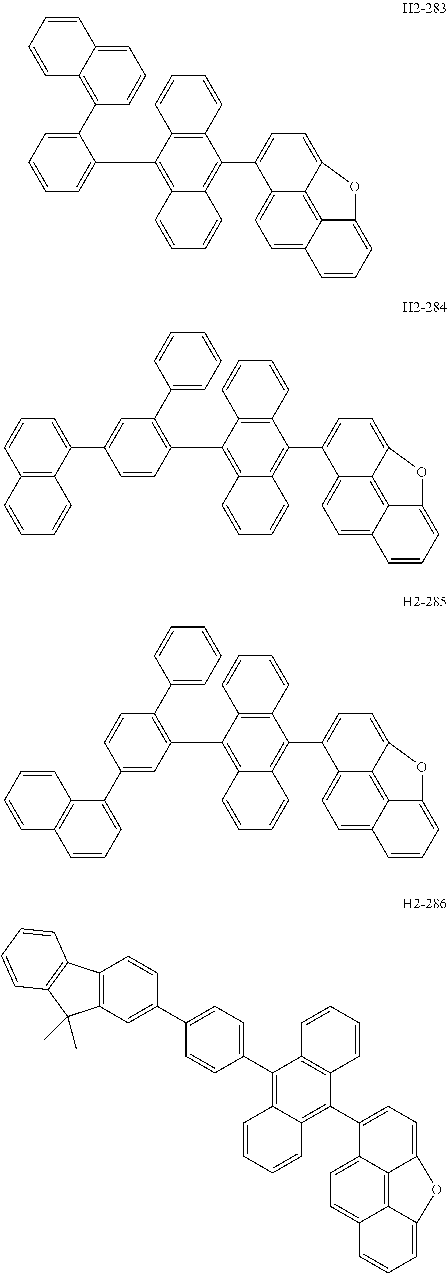 [SMILES: c1ccc(-c2c3ccccc3c(-c3ccc4oc5cccc6ccc3c4c65)c3ccccc23)c(-c2cccc3ccccc23)c1 ; c1ccc(-c2cc(-c3cccc4ccccc34)ccc2-c2c3ccccc3c(-c3ccc4oc5cccc6ccc3c4c65)c3ccccc23)cc1 ; c1ccc(-c2ccc(-c3cccc4ccccc34)cc2-c2c3ccccc3c(-c3ccc4oc5cccc6ccc3c4c65)c3ccccc23)cc1 ; CC1(C)c2ccccc2-c2ccc(-c3ccc(-c4c5ccccc5c(-c5ccc6oc7cccc8ccc5c6c87)c5ccccc45)cc3)cc21] 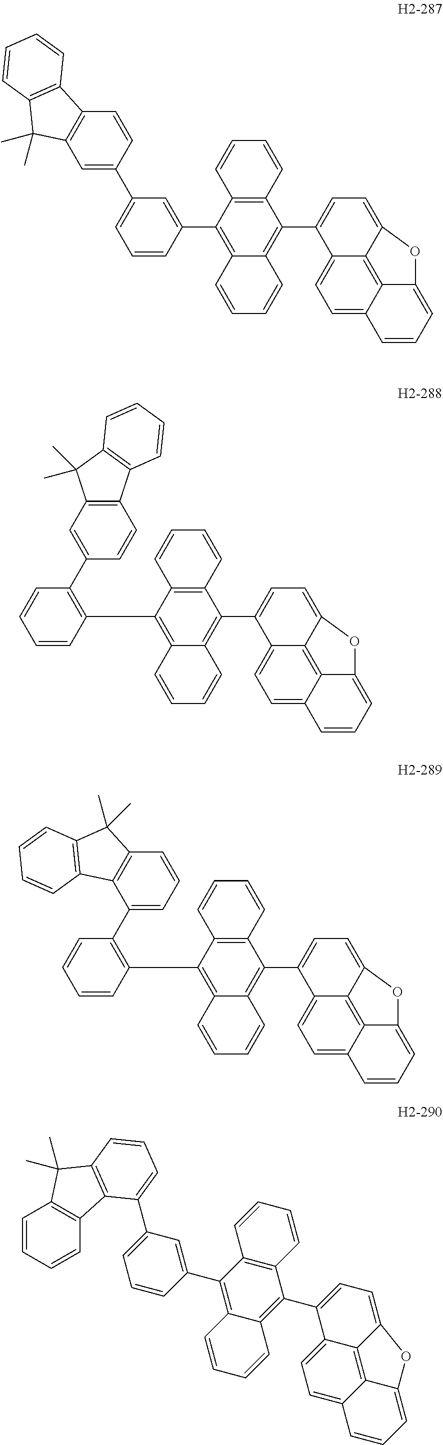 [SMILES: CC1(C)c2ccccc2-c2ccc(-c3cccc(-c4c5ccccc5c(-c5ccc6oc7cccc8ccc5c6c87)c5ccccc45)c3)cc21 ; CC1(C)c2ccccc2-c2ccc(-c3ccccc3-c3c4ccccc4c(-c4ccc5oc6cccc7ccc4c5c76)c4ccccc34)cc21 ; CC1(C)c2ccccc2-c2c(-c3ccccc3-c3c4ccccc4c(-c4ccc5oc6cccc7ccc4c5c76)c4ccccc34)cccc21 ; CC1(C)c2ccccc2-c2c(-c3cccc(-c4c5ccccc5c(-c5ccc6oc7cccc8ccc5c6c87)c5ccccc45)c3)cccc21] 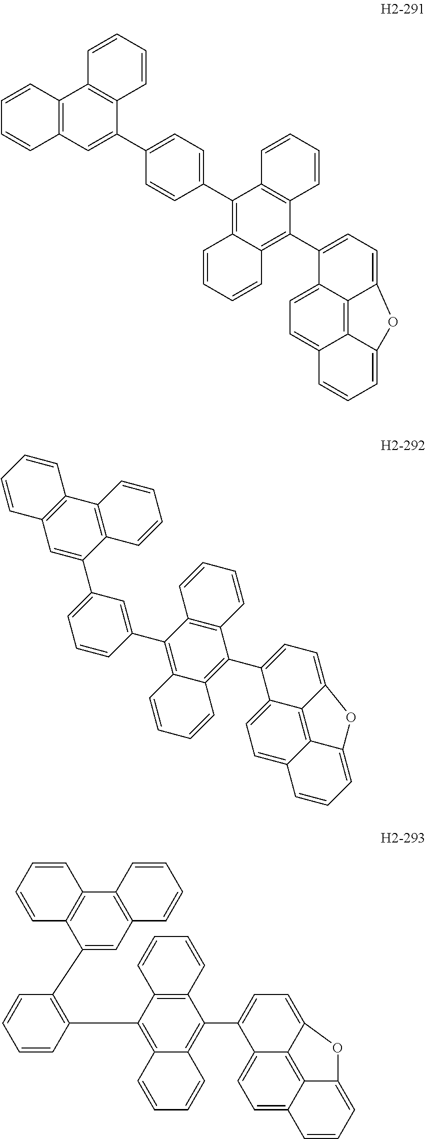 [SMILES: c1ccc2c(c1)cc(-c1ccc(-c3c4ccccc4c(-c4ccc5oc6cccc7ccc4c5c76)c4ccccc34)cc1)c1ccccc12 ; c1cc(-c2c3ccccc3c(-c3ccc4oc5cccc6ccc3c4c65)c3ccccc23)cc(-c2cc3ccccc3c3ccccc23)c1 ; c1ccc(-c2cc3ccccc3c3ccccc23)c(-c2c3ccccc3c(-c3ccc4oc5cccc6ccc3c4c65)c3ccccc23)c1] 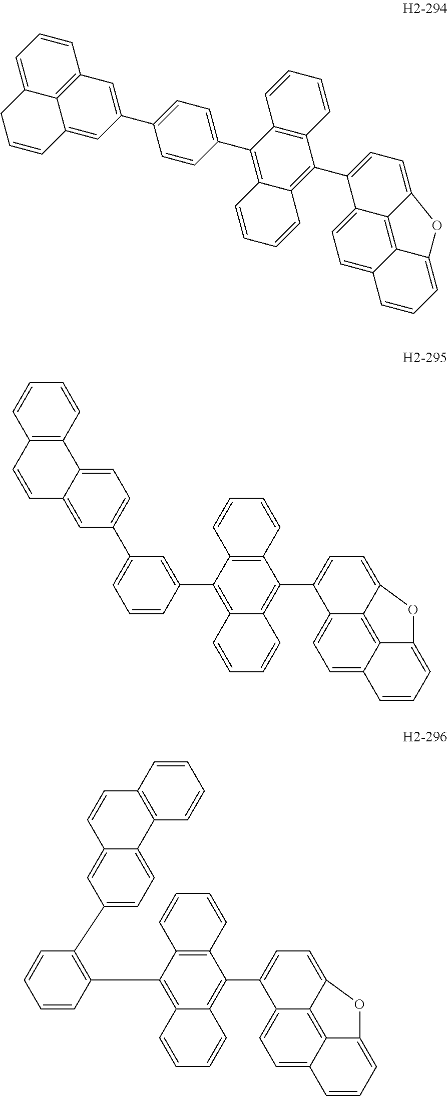 [SMILES: C1=Cc2cc(-c3ccc(-c4c5ccccc5c(-c5ccc6oc7cccc8ccc5c6c87)c5ccccc45)cc3)cc3cccc(c23)C1 ; c1cc(-c2ccc3c(ccc4ccccc43)c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(ccc4ccccc43)c2)c1] 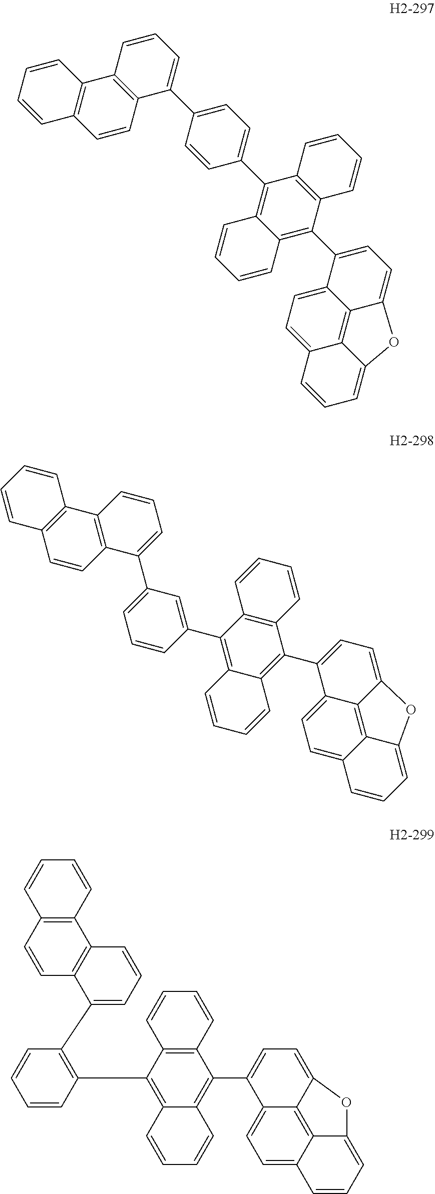 [SMILES: c1ccc2c(c1)ccc1c(-c3ccc(-c4c5ccccc5c(-c5ccc6oc7cccc8ccc5c6c87)c5ccccc45)cc3)cccc12 ; c1cc(-c2c3ccccc3c(-c3ccc4oc5cccc6ccc3c4c65)c3ccccc23)cc(-c2cccc3c2ccc2ccccc23)c1 ; c1ccc(-c2cccc3c2ccc2ccccc23)c(-c2c3ccccc3c(-c3ccc4oc5cccc6ccc3c4c65)c3ccccc23)c1] 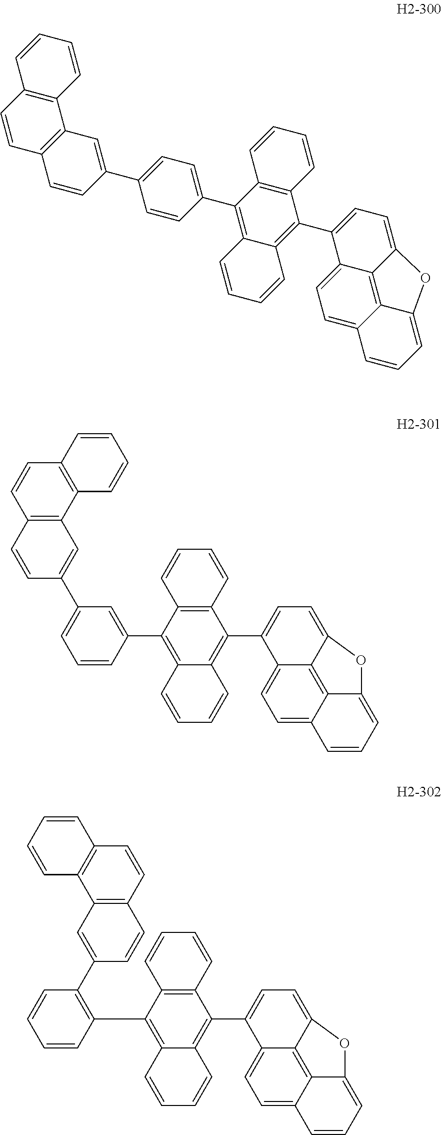 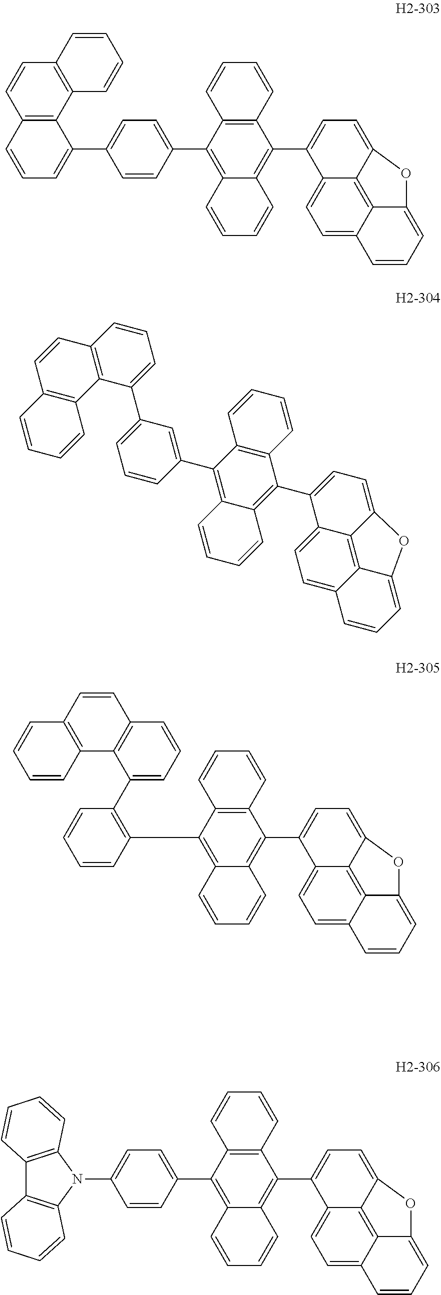 [SMILES: c1ccc2c(c1)ccc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3ccc4ccccc4c23)c1 ; c1ccc(-c2cccc3ccc4ccccc4c23)c(-c2c3ccccc3c(-c3ccc4oc5cccc6ccc3c4c65)c3ccccc23)c1 ; c1cc2ccc3c(-c4c5ccccc5c(-c5ccc(-n6c7ccccc7c7ccccc76)cc5)c5ccccc45)ccc4oc(c1)c2c43] 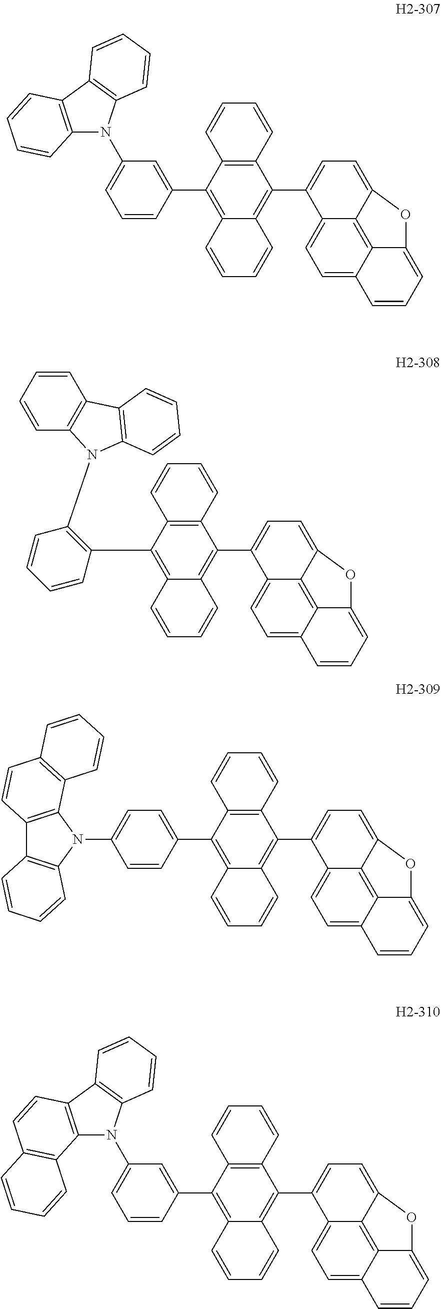 [SMILES: c1cc(-c2c3ccccc3c(-c3ccc4oc5cccc6ccc3c4c65)c3ccccc23)cc(-n2c3ccccc3c3ccccc32)c1 ; c1ccc(-n2c3ccccc3c3ccccc32)c(-c2c3ccccc3c(-c3ccc4oc5cccc6ccc3c4c65)c3ccccc23)c1 ; c1ccc2c(c1)ccc1c3ccccc3n(-c3ccc(-c4c5ccccc5c(-c5ccc6oc7cccc8ccc5c6c87)c5ccccc45)cc3)c21 ; c1cc(-c2c3ccccc3c(-c3ccc4oc5cccc6ccc3c4c65)c3ccccc23)cc(-n2c3ccccc3c3ccc4ccccc4c32)c1] 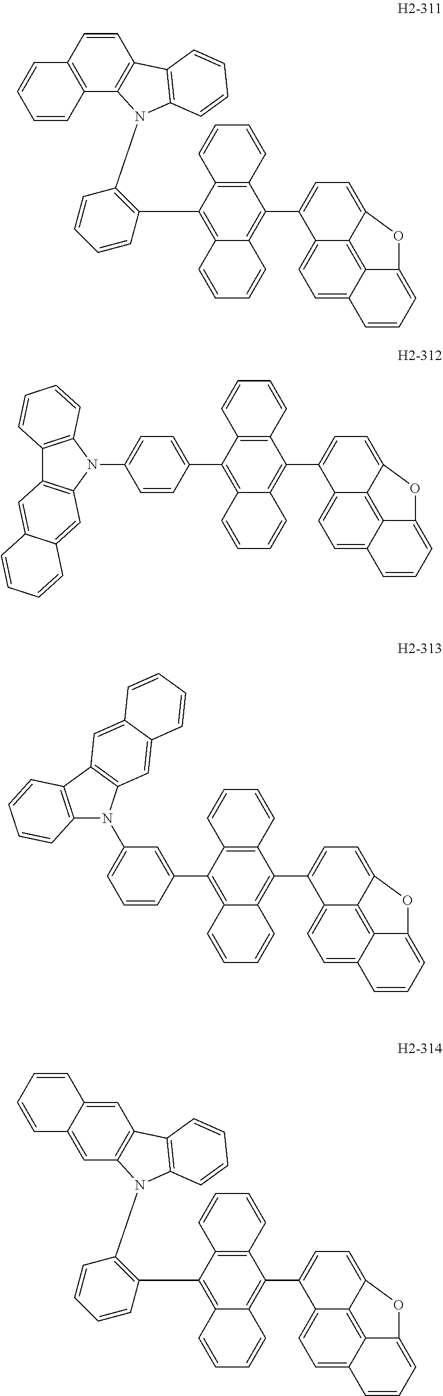 [SMILES: c1ccc(-n2c3ccccc3c3ccc4ccccc4c32)c(-c2c3ccccc3c(-c3ccc4oc5cccc6ccc3c4c65)c3ccccc23)c1 ; c1ccc2cc3c(cc2c1)c1ccccc1n3-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3cc4ccccc4cc32)c1 ; c1ccc(-n2c3ccccc3c3cc4ccccc4cc32)c(-c2c3ccccc3c(-c3ccc4oc5cccc6ccc3c4c65)c3ccccc23)c1] 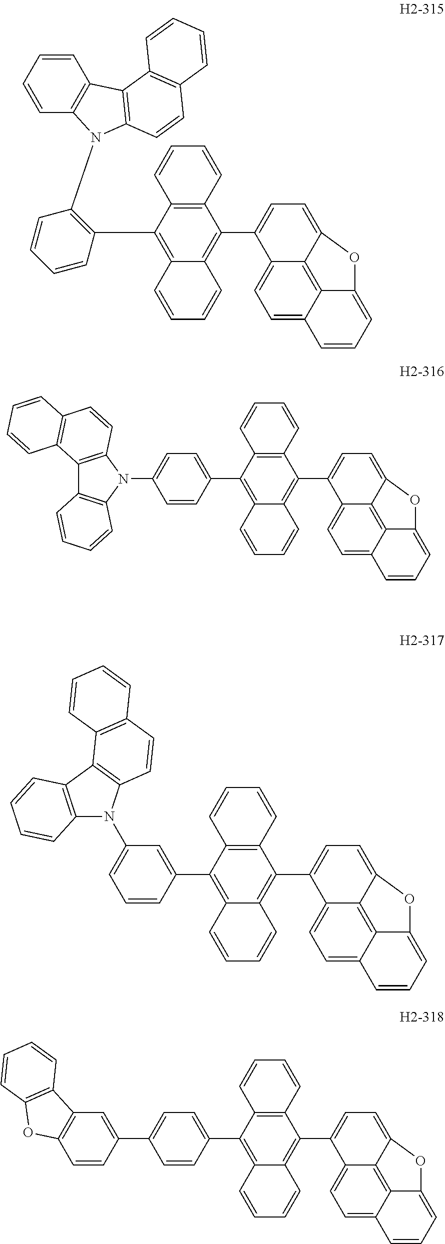 [SMILES: c1ccc(-n2c3ccccc3c3c4ccccc4ccc32)c(-c2c3ccccc3c(-c3ccc4oc5cccc6ccc3c4c65)c3ccccc23)c1 ; c1ccc2c(c1)ccc1c2c2ccccc2n1-c1ccc(-c2c3ccccc3c(-c3ccc4oc5cccc6ccc3c4c65)c3ccccc23)cc1 ; c1cc(-c2c3ccccc3c(-c3ccc4oc5cccc6ccc3c4c65)c3ccccc23)cc(-n2c3ccccc3c3c4ccccc4ccc32)c1 ; c1ccc2c(c1)oc1ccc(-c3ccc(-c4c5ccccc5c(-c5ccc6oc7cccc8ccc5c6c87)c5ccccc45)cc3)cc12] 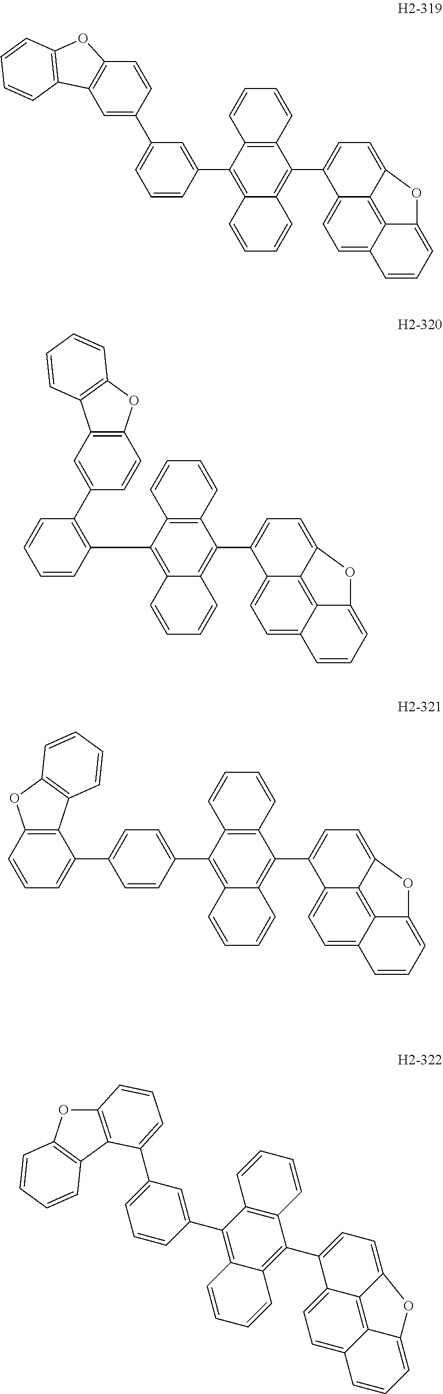 [SMILES: c1cc(-c2ccc3oc4ccccc4c3c2)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3oc4ccccc4c3c2)c1 ; c1ccc2c(c1)oc1cccc(-c3ccc(-c4c5ccccc5c(-c5ccc6oc7cccc8ccc5c6c87)c5ccccc45)cc3)c12 ; c1cc(-c2c3ccccc3c(-c3ccc4oc5cccc6ccc3c4c65)c3ccccc23)cc(-c2cccc3oc4ccccc4c23)c1] 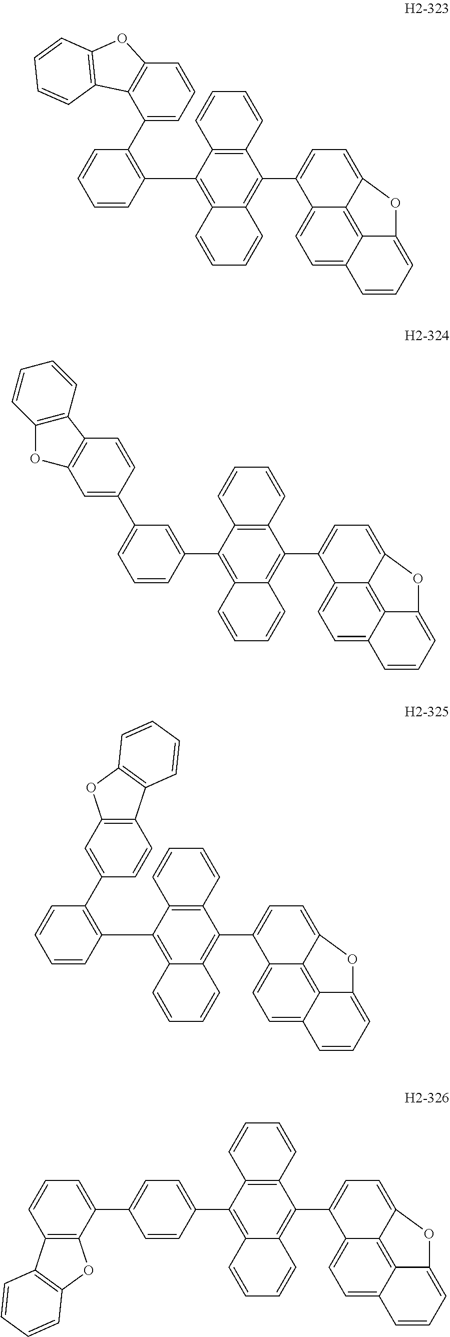 [SMILES: c1ccc(-c2cccc3oc4ccccc4c23)c(-c2c3ccccc3c(-c3ccc4oc5cccc6ccc3c4c65)c3ccccc23)c1 ; c1cc(-c2ccc3c(c2)oc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)oc2ccccc23)c1 ; c1ccc2c(c1)oc1c(-c3ccc(-c4c5ccccc5c(-c5ccc6oc7cccc8ccc5c6c87)c5ccccc45)cc3)cccc12] 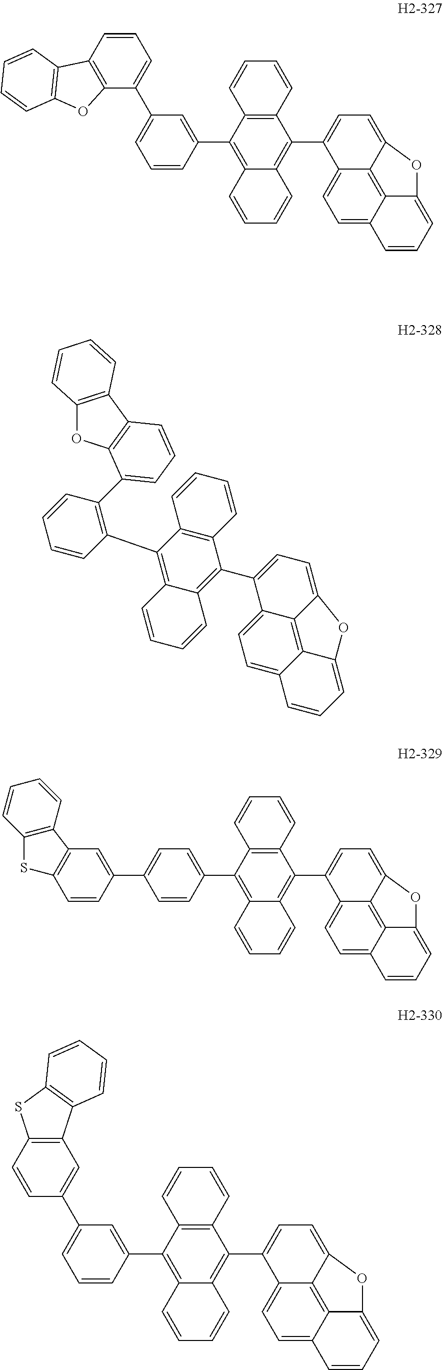 [SMILES: c1cc(-c2c3ccccc3c(-c3ccc4oc5cccc6ccc3c4c65)c3ccccc23)cc(-c2cccc3c2oc2ccccc23)c1 ; c1ccc(-c2cccc3c2oc2ccccc23)c(-c2c3ccccc3c(-c3ccc4oc5cccc6ccc3c4c65)c3ccccc23)c1 ; c1cc2ccc3c(-c4c5ccccc5c(-c5ccc(-c6ccc7sc8ccccc8c7c6)cc5)c5ccccc45)ccc4oc(c1)c2c43 ; c1cc(-c2ccc3sc4ccccc4c3c2)cc(-c2c3ccccc3c(-c3ccc4oc5cccc6ccc3c4c65)c3ccccc23)c1] 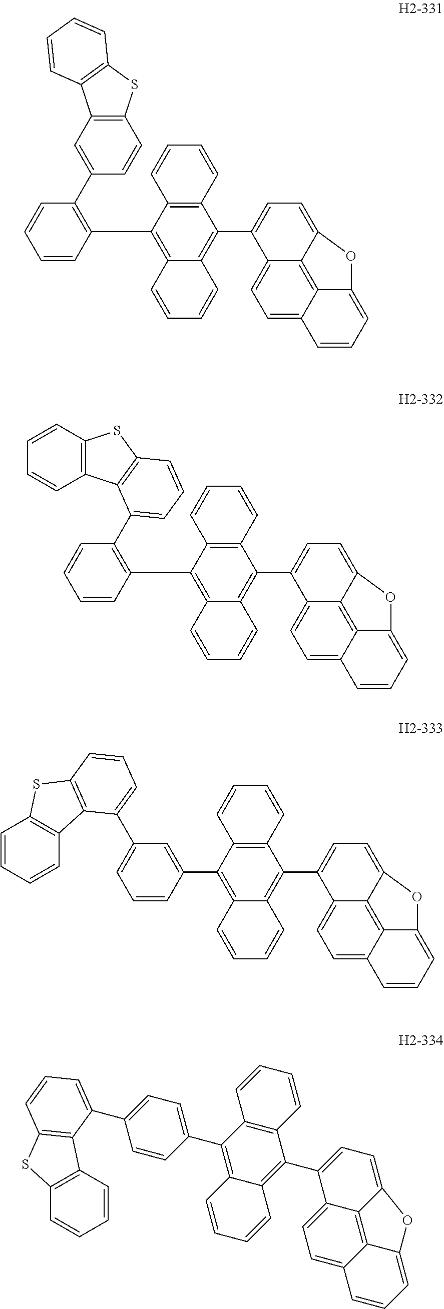 [SMILES: c1ccc(-c2c3ccccc3c(-c3ccc4oc5cccc6ccc3c4c65)c3ccccc23)c(-c2ccc3sc4ccccc4c3c2)c1 ; c1ccc(-c2cccc3sc4ccccc4c23)c(-c2c3ccccc3c(-c3ccc4oc5cccc6ccc3c4c65)c3ccccc23)c1 ; c1cc(-c2c3ccccc3c(-c3ccc4oc5cccc6ccc3c4c65)c3ccccc23)cc(-c2cccc3sc4ccccc4c23)c1 ; c1cc2ccc3c(-c4c5ccccc5c(-c5ccc(-c6cccc7sc8ccccc8c67)cc5)c5ccccc45)ccc4oc(c1)c2c43] 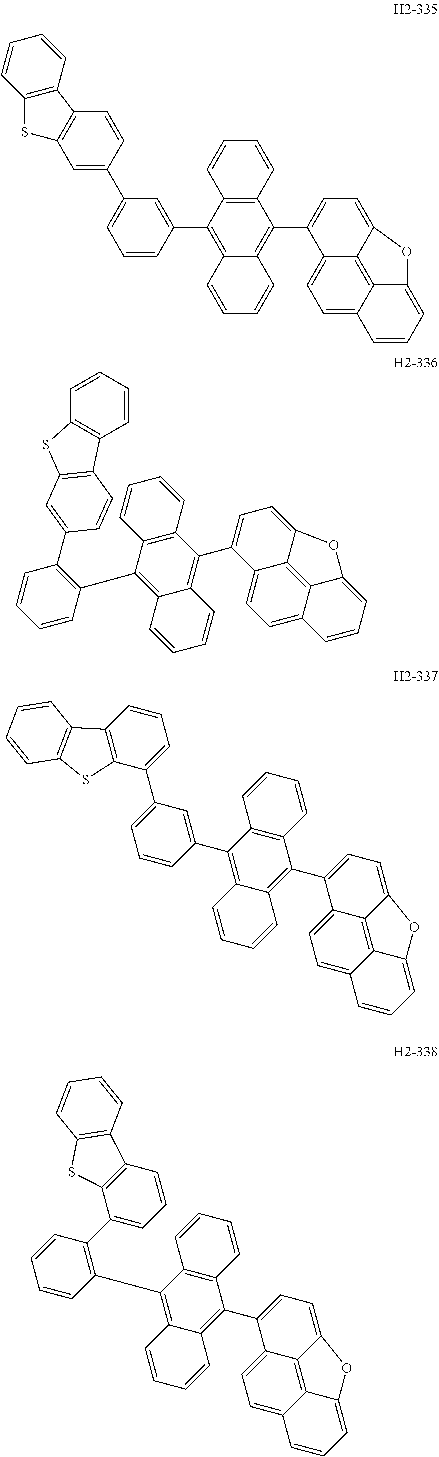 [SMILES: c1cc(-c2ccc3c(c2)sc2ccccc23)cc(-c2c3ccccc3c(-c3ccc4oc5cccc6ccc3c4c65)c3ccccc23)c1 ; c1ccc(-c2c3ccccc3c(-c3ccc4oc5cccc6ccc3c4c65)c3ccccc23)c(-c2ccc3c(c2)sc2ccccc23)c1 ; c1cc(-c2c3ccccc3c(-c3ccc4oc5cccc6ccc3c4c65)c3ccccc23)cc(-c2cccc3c2sc2ccccc23)c1 ; c1ccc(-c2cccc3c2sc2ccccc23)c(-c2c3ccccc3c(-c3ccc4oc5cccc6ccc3c4c65)c3ccccc23)c1] 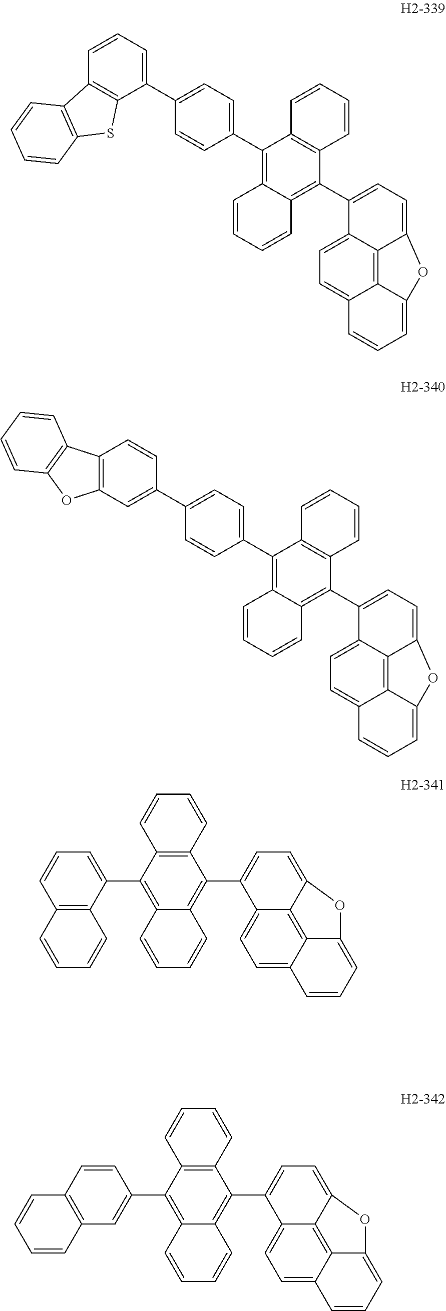 [SMILES: c1cc2ccc3c(-c4c5ccccc5c(-c5ccc(-c6cccc7c6sc6ccccc67)cc5)c5ccccc45)ccc4oc(c1)c2c43 ; c1ccc2c(c1)oc1cc(-c3ccc(-c4c5ccccc5c(-c5ccc6oc7cccc8ccc5c6c87)c5ccccc45)cc3)ccc12 ; c1ccc2c(-c3c4ccccc4c(-c4ccc5oc6cccc7ccc4c5c76)c4ccccc34)cccc2c1 ; c1ccc2cc(-c3c4ccccc4c(-c4ccc5oc6cccc7ccc4c5c76)c4ccccc34)ccc2c1] 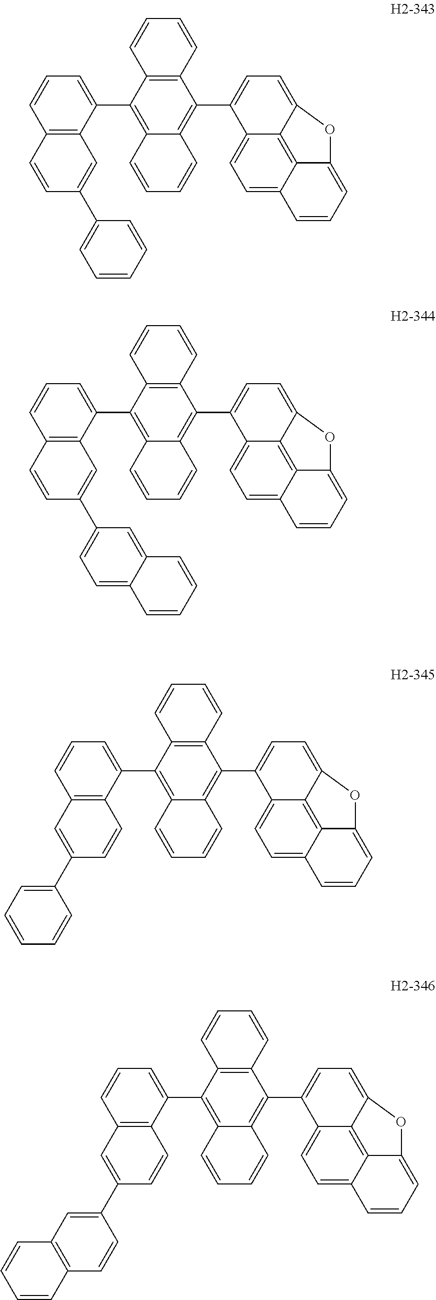 [SMILES: c1ccc(-c2ccc3cccc(-c4c5ccccc5c(-c5ccc6oc7cccc8ccc5c6c87)c5ccccc45)c3c2)cc1 ; c1ccc2cc(-c3ccc4cccc(-c5c6ccccc6c(-c6ccc7oc8cccc9ccc6c7c98)c6ccccc56)c4c3)ccc2c1 ; c1ccc(-c2ccc3c(-c4c5ccccc5c(-c5ccc6oc7cccc8ccc5c6c87)c5ccccc45)cccc3c2)cc1 ; c1ccc2cc(-c3ccc4c(-c5c6ccccc6c(-c6ccc7oc8cccc9ccc6c7c98)c6ccccc56)cccc4c3)ccc2c1] 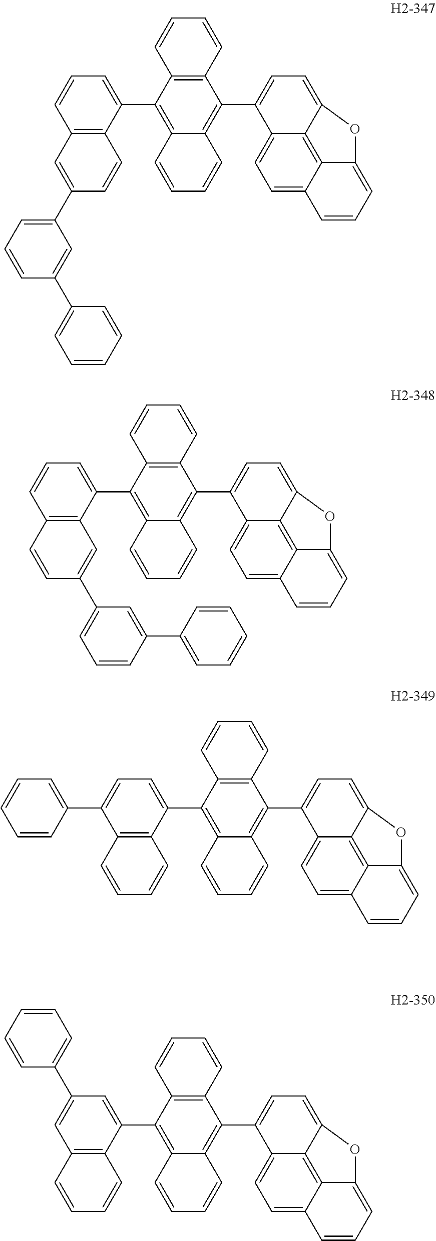 [SMILES: c1ccc(-c2cccc(-c3ccc4c(-c5c6ccccc6c(-c6ccc7oc8cccc9ccc6c7c98)c6ccccc56)cccc4c3)c2)cc1 ; c1ccc(-c2cccc(-c3ccc4cccc(-c5c6ccccc6c(-c6ccc7oc8cccc9ccc6c7c98)c6ccccc56)c4c3)c2)cc1 ; c1ccc(-c2ccc(-c3c4ccccc4c(-c4ccc5oc6cccc7ccc4c5c76)c4ccccc34)c3ccccc23)cc1 ; c1ccc(-c2cc(-c3c4ccccc4c(-c4ccc5oc6cccc7ccc4c5c76)c4ccccc34)c3ccccc3c2)cc1] 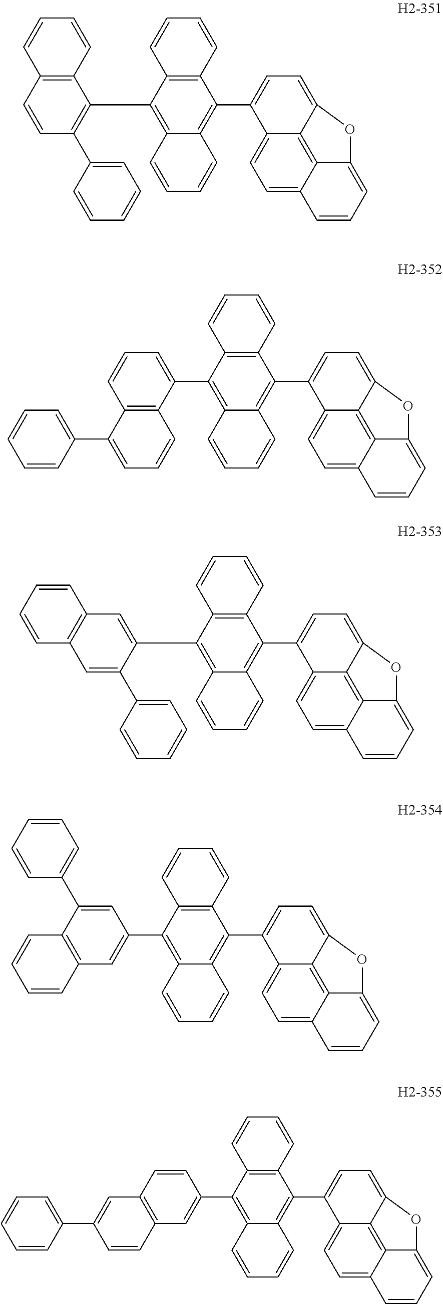 [SMILES: c1ccc(-c2ccc3ccccc3c2-c2c3ccccc3c(-c3ccc4oc5cccc6ccc3c4c65)c3ccccc23)cc1 ; c1ccc(-c2cccc3c(-c4c5ccccc5c(-c5ccc6oc7cccc8ccc5c6c87)c5ccccc45)cccc23)cc1 ; c1ccc(-c2cc3ccccc3cc2-c2c3ccccc3c(-c3ccc4oc5cccc6ccc3c4c65)c3ccccc23)cc1 ; c1ccc(-c2cc(-c3c4ccccc4c(-c4ccc5oc6cccc7ccc4c5c76)c4ccccc34)cc3ccccc23)cc1 ; c1ccc(-c2ccc3cc(-c4c5ccccc5c(-c5ccc6oc7cccc8ccc5c6c87)c5ccccc45)ccc3c2)cc1] 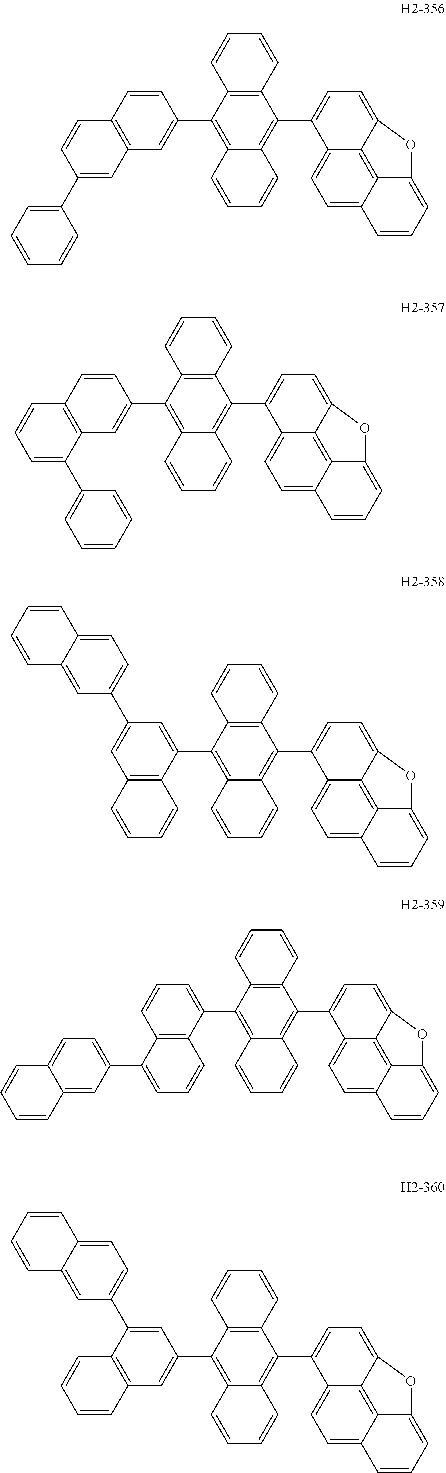 [SMILES: c1ccc(-c2ccc3ccc(-c4c5ccccc5c(-c5ccc6oc7cccc8ccc5c6c87)c5ccccc45)cc3c2)cc1 ; c1ccc(-c2cccc3ccc(-c4c5ccccc5c(-c5ccc6oc7cccc8ccc5c6c87)c5ccccc45)cc23)cc1 ; c1ccc2cc(-c3cc(-c4c5ccccc5c(-c5ccc6oc7cccc8ccc5c6c87)c5ccccc45)c4ccccc4c3)ccc2c1 ; c1ccc2cc(-c3cccc4c(-c5c6ccccc6c(-c6ccc7oc8cccc9ccc6c7c98)c6ccccc56)cccc34)ccc2c1 ; c1ccc2cc(-c3cc(-c4c5ccccc5c(-c5ccc6oc7cccc8ccc5c6c87)c5ccccc45)cc4ccccc34)ccc2c1] 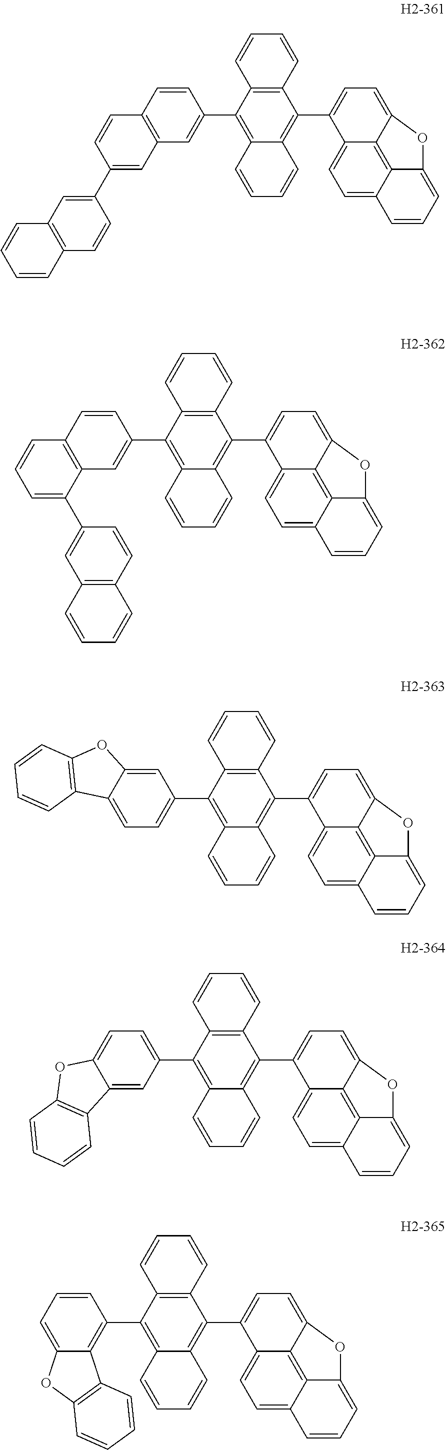 [SMILES: c1ccc2cc(-c3ccc4ccc(-c5c6ccccc6c(-c6ccc7oc8cccc9ccc6c7c98)c6ccccc56)cc4c3)ccc2c1 ; c1ccc2cc(-c3cccc4ccc(-c5c6ccccc6c(-c6ccc7oc8cccc9ccc6c7c98)c6ccccc56)cc34)ccc2c1 ; c1ccc2c(c1)oc1cc(-c3c4ccccc4c(-c4ccc5oc6cccc7ccc4c5c76)c4ccccc34)ccc12 ; c1ccc2c(c1)oc1ccc(-c3c4ccccc4c(-c4ccc5oc6cccc7ccc4c5c76)c4ccccc34)cc12 ; c1ccc2c(c1)oc1cccc(-c3c4ccccc4c(-c4ccc5oc6cccc7ccc4c5c76)c4ccccc34)c12] 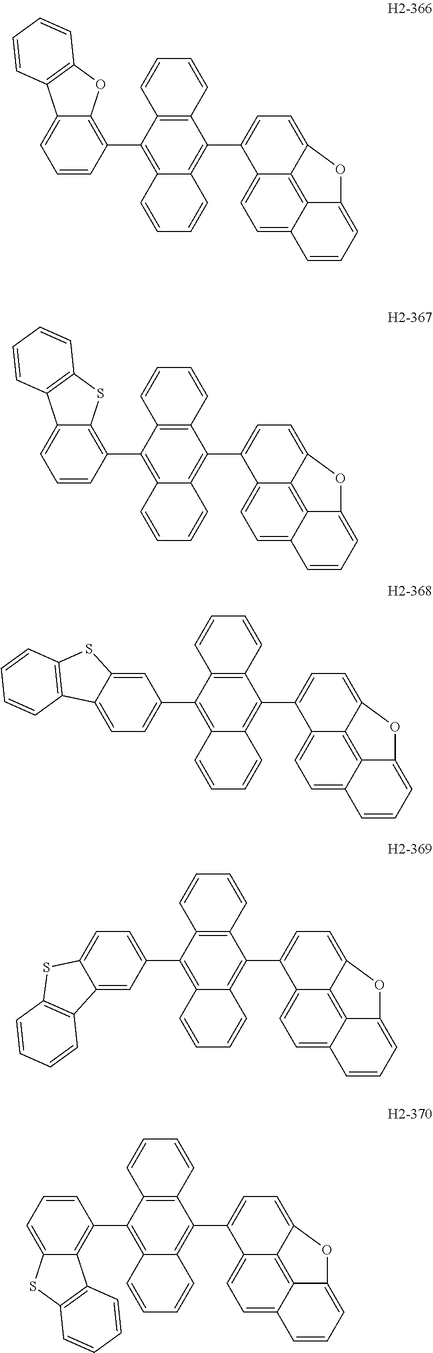 [SMILES: c1ccc2c(c1)oc1c(-c3c4ccccc4c(-c4ccc5oc6cccc7ccc4c5c76)c4ccccc34)cccc12 ; c1cc2ccc3c(-c4c5ccccc5c(-c5cccc6c5sc5ccccc56)c5ccccc45)ccc4oc(c1)c2c43 ; c1cc2ccc3c(-c4c5ccccc5c(-c5ccc6c(c5)sc5ccccc56)c5ccccc45)ccc4oc(c1)c2c43 ; c1cc2ccc3c(-c4c5ccccc5c(-c5ccc6sc7ccccc7c6c5)c5ccccc45)ccc4oc(c1)c2c43 ; c1cc2ccc3c(-c4c5ccccc5c(-c5cccc6sc7ccccc7c56)c5ccccc45)ccc4oc(c1)c2c43] 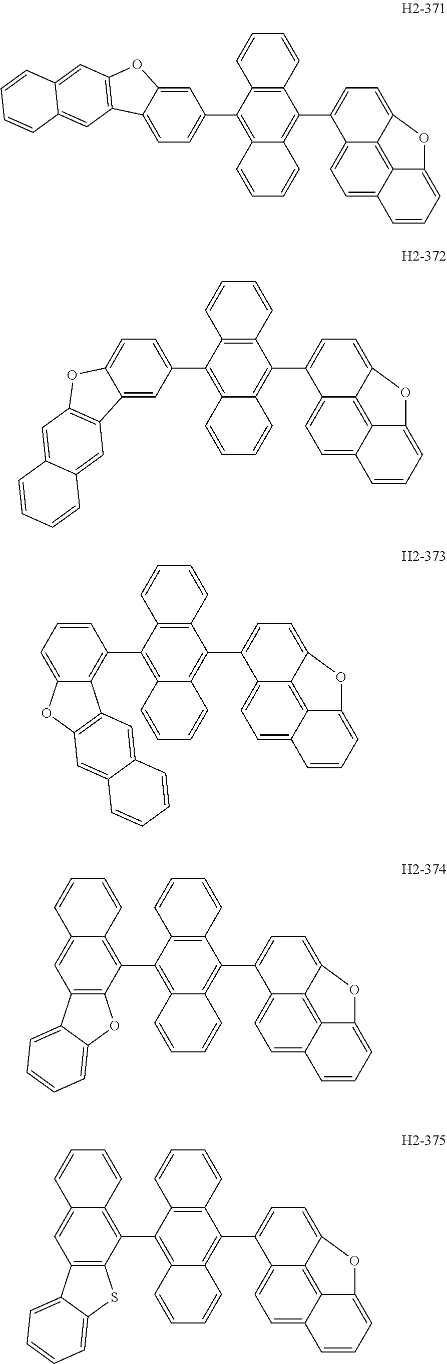 [SMILES: c1ccc2cc3c(cc2c1)oc1cc(-c2c4ccccc4c(-c4ccc5oc6cccc7ccc4c5c76)c4ccccc24)ccc13 ; c1ccc2cc3c(cc2c1)oc1ccc(-c2c4ccccc4c(-c4ccc5oc6cccc7ccc4c5c76)c4ccccc24)cc13 ; c1ccc2cc3c(cc2c1)oc1cccc(-c2c4ccccc4c(-c4ccc5oc6cccc7ccc4c5c76)c4ccccc24)c13 ; c1ccc2c(-c3c4ccccc4c(-c4ccc5oc6cccc7ccc4c5c76)c4ccccc34)c3oc4ccccc4c3cc2c1 ; c1ccc2c(-c3c4ccccc4c(-c4ccc5oc6cccc7ccc4c5c76)c4ccccc34)c3sc4ccccc4c3cc2c1] 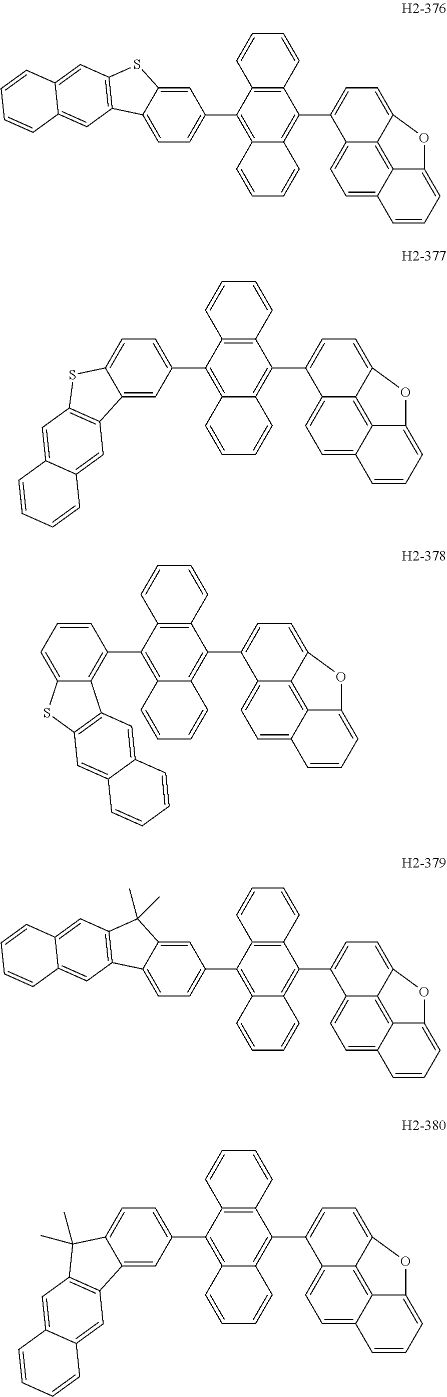 [SMILES: c1ccc2cc3c(cc2c1)sc1cc(-c2c4ccccc4c(-c4ccc5oc6cccc7ccc4c5c76)c4ccccc24)ccc13 ; c1ccc2cc3c(cc2c1)sc1ccc(-c2c4ccccc4c(-c4ccc5oc6cccc7ccc4c5c76)c4ccccc24)cc13 ; c1ccc2cc3c(cc2c1)sc1cccc(-c2c4ccccc4c(-c4ccc5oc6cccc7ccc4c5c76)c4ccccc24)c13 ; CC1(C)c2cc(-c3c4ccccc4c(-c4ccc5oc6cccc7ccc4c5c76)c4ccccc34)ccc2-c2cc3ccccc3cc21 ; CC1(C)c2ccc(-c3c4ccccc4c(-c4ccc5oc6cccc7ccc4c5c76)c4ccccc34)cc2-c2cc3ccccc3cc21] 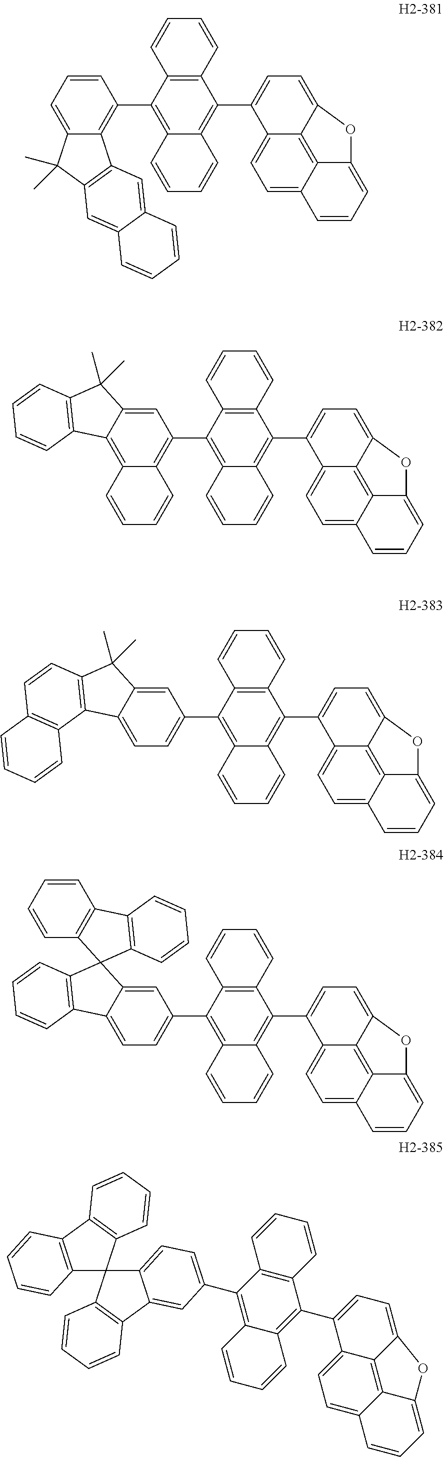 [SMILES: CC1(C)c2cc3ccccc3cc2-c2c(-c3c4ccccc4c(-c4ccc5oc6cccc7ccc4c5c76)c4ccccc34)cccc21 ; CC1(C)c2ccccc2-c2c1cc(-c1c3ccccc3c(-c3ccc4oc5cccc6ccc3c4c65)c3ccccc13)c1ccccc21 ; CC1(C)c2cc(-c3c4ccccc4c(-c4ccc5oc6cccc7ccc4c5c76)c4ccccc34)ccc2-c2c1ccc1ccccc21 ; c1ccc2c(c1)-c1ccccc1C21c2ccccc2-c2ccc(-c3c4ccccc4c(-c4ccc5oc6cccc7ccc4c5c76)c4ccccc34)cc21 ; c1ccc2c(c1)-c1ccccc1C21c2ccccc2-c2cc(-c3c4ccccc4c(-c4ccc5oc6cccc7ccc4c5c76)c4ccccc34)ccc21] 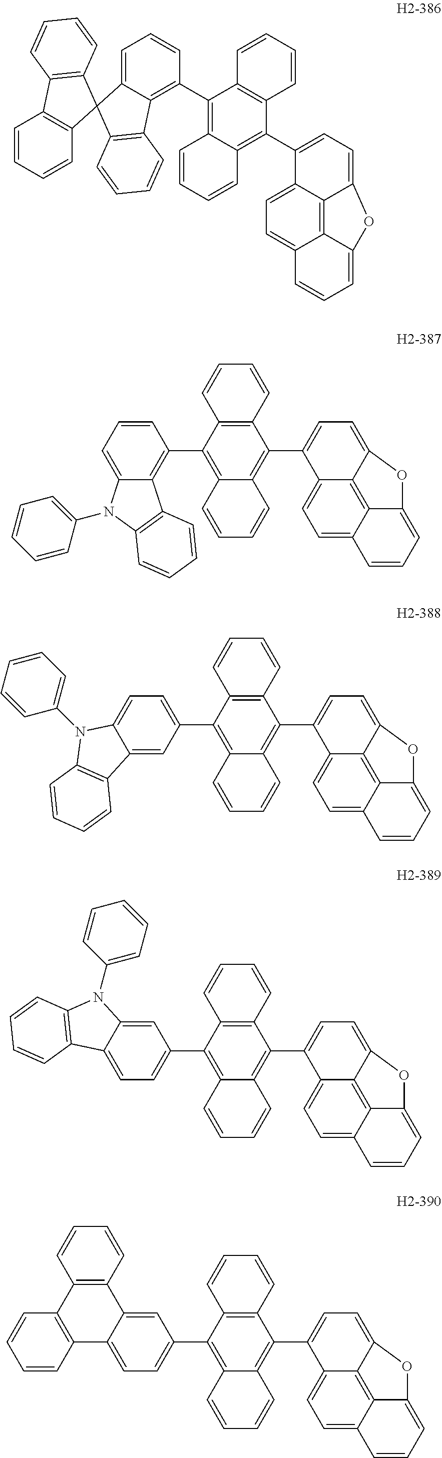 [SMILES: c1ccc2c(c1)-c1ccccc1C21c2ccccc2-c2c(-c3c4ccccc4c(-c4ccc5oc6cccc7ccc4c5c76)c4ccccc34)cccc21 ; c1ccc(-n2c3ccccc3c3c(-c4c5ccccc5c(-c5ccc6oc7cccc8ccc5c6c87)c5ccccc45)cccc32)cc1 ; c1ccc(-n2c3ccccc3c3cc(-c4c5ccccc5c(-c5ccc6oc7cccc8ccc5c6c87)c5ccccc45)ccc32)cc1 ; c1ccc(-n2c3ccccc3c3ccc(-c4c5ccccc5c(-c5ccc6oc7cccc8ccc5c6c87)c5ccccc45)cc32)cc1 ; c1cc2ccc3c(-c4c5ccccc5c(-c5ccc6c7ccccc7c7ccccc7c6c5)c5ccccc45)ccc4oc(c1)c2c43] 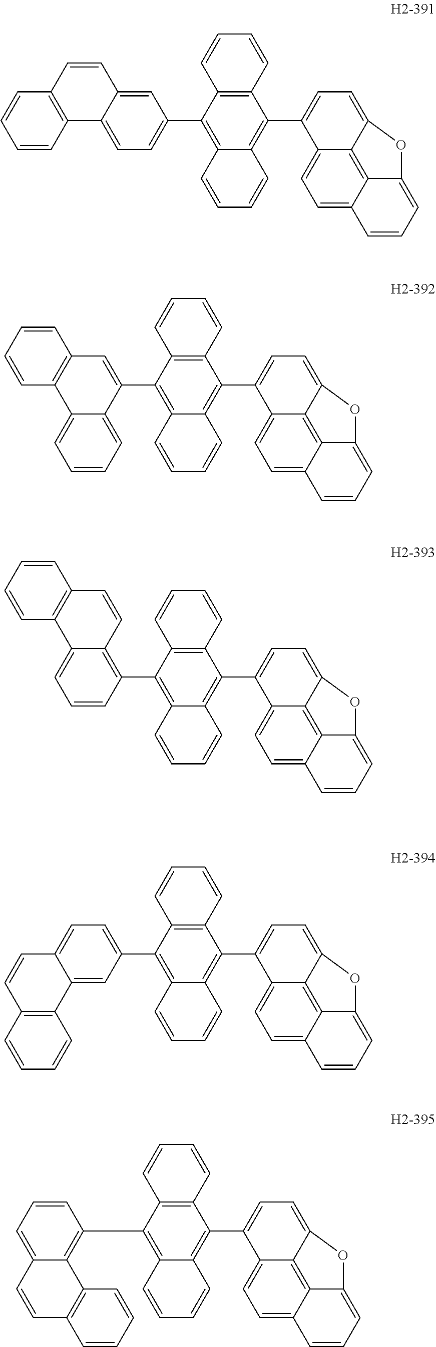 [SMILES: c1ccc2c(c1)ccc1cc(-c3c4ccccc4c(-c4ccc5oc6cccc7ccc4c5c76)c4ccccc34)ccc12 ; c1ccc2c(c1)cc(-c1c3ccccc3c(-c3ccc4oc5cccc6ccc3c4c65)c3ccccc13)c1ccccc12 ; c1ccc2c(c1)ccc1c(-c3c4ccccc4c(-c4ccc5oc6cccc7ccc4c5c76)c4ccccc34)cccc12 ; c1ccc2c(c1)ccc1ccc(-c3c4ccccc4c(-c4ccc5oc6cccc7ccc4c5c76)c4ccccc34)cc12 ; c1ccc2c(c1)ccc1cccc(-c3c4ccccc4c(-c4ccc5oc6cccc7ccc4c5c76)c4ccccc34)c12] 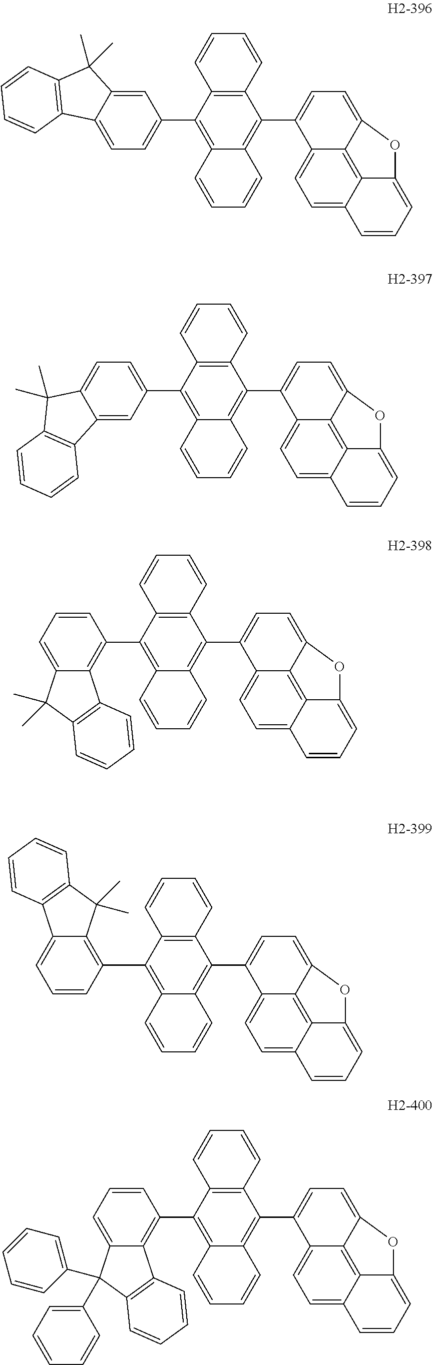 [SMILES: CC1(C)c2ccccc2-c2ccc(-c3c4ccccc4c(-c4ccc5oc6cccc7ccc4c5c76)c4ccccc34)cc21 ; CC1(C)c2ccccc2-c2cc(-c3c4ccccc4c(-c4ccc5oc6cccc7ccc4c5c76)c4ccccc34)ccc21 ; CC1(C)c2ccccc2-c2c(-c3c4ccccc4c(-c4ccc5oc6cccc7ccc4c5c76)c4ccccc34)cccc21 ; CC1(C)c2ccccc2-c2cccc(-c3c4ccccc4c(-c4ccc5oc6cccc7ccc4c5c76)c4ccccc34)c21 ; c1ccc(C2(c3ccccc3)c3ccccc3-c3c(-c4c5ccccc5c(-c5ccc6oc7cccc8ccc5c6c87)c5ccccc45)cccc32)cc1] 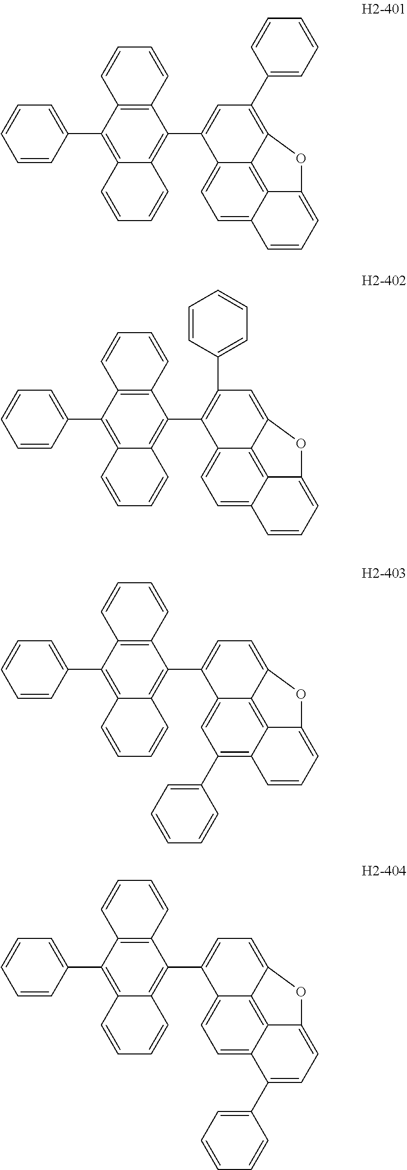 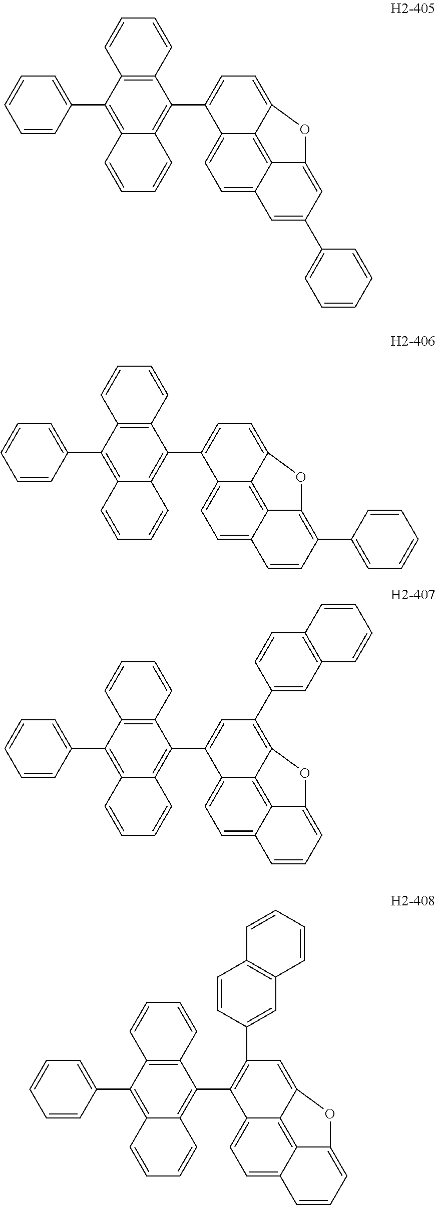 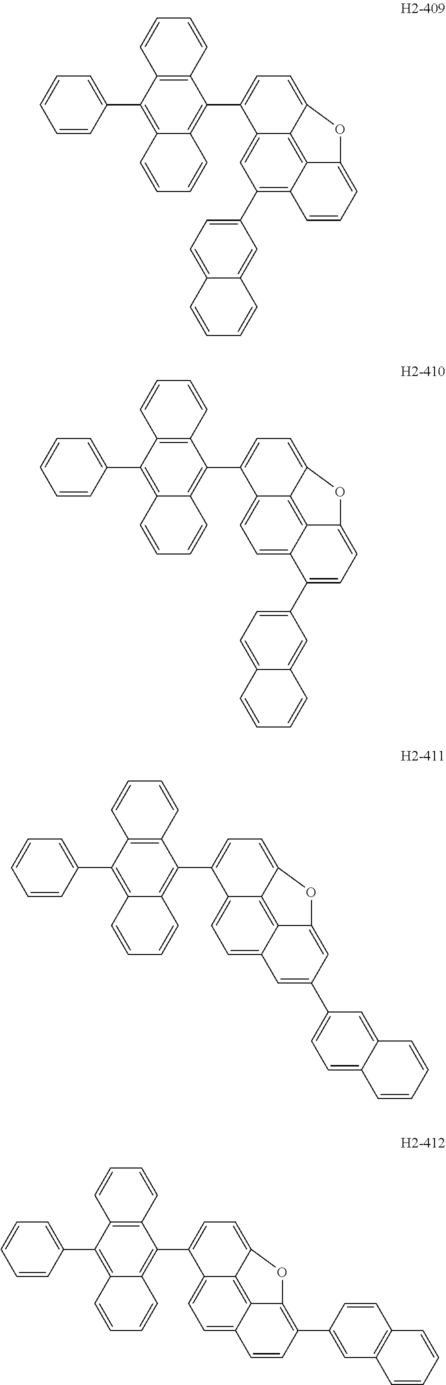 [SMILES: c1ccc(-c2c3ccccc3c(-c3ccc4oc5cccc6c(-c7ccc8ccccc8c7)cc3c4c56)c3ccccc23)cc1 ; c1ccc(-c2c3ccccc3c(-c3ccc4oc5ccc(-c6ccc7ccccc7c6)c6ccc3c4c56)c3ccccc23)cc1 ; c1ccc(-c2c3ccccc3c(-c3ccc4oc5cc(-c6ccc7ccccc7c6)cc6ccc3c4c65)c3ccccc23)cc1 ; c1ccc(-c2c3ccccc3c(-c3ccc4oc5c(-c6ccc7ccccc7c6)ccc6ccc3c4c65)c3ccccc23)cc1] 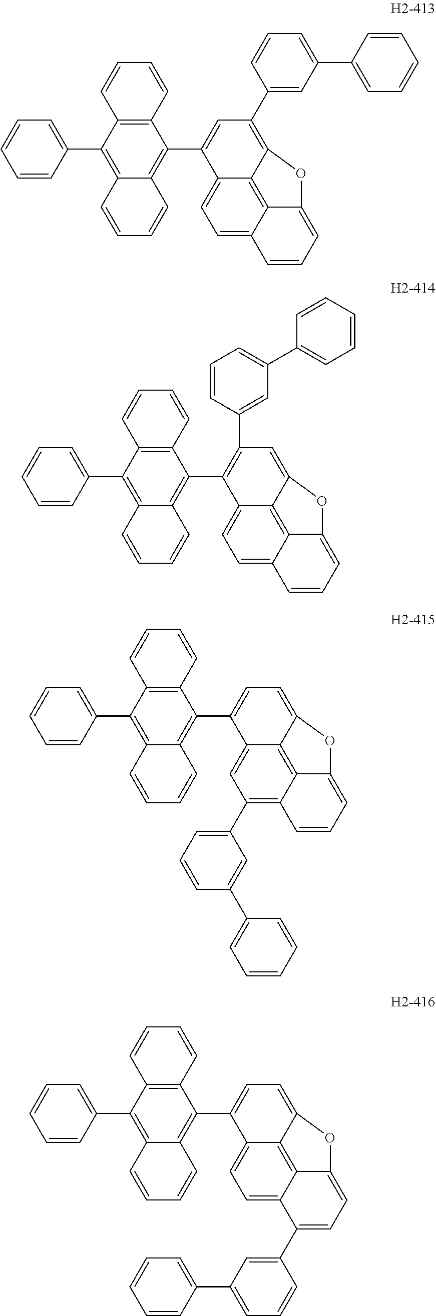 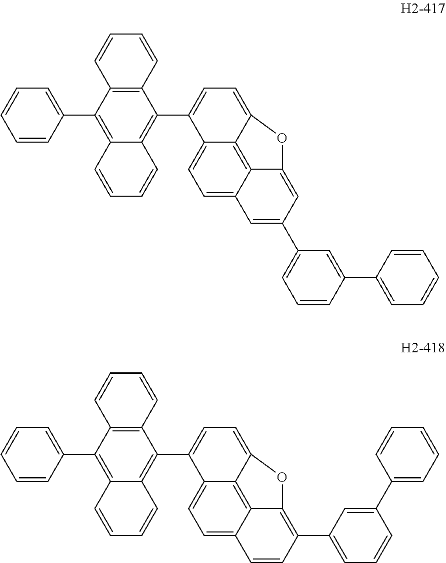 [SMILES: c1ccc(-c2cccc(-c3cc4ccc5c(-c6c7ccccc7c(-c7ccccc7)c7ccccc67)ccc6oc(c3)c4c65)c2)cc1 ; c1ccc(-c2cccc(-c3ccc4ccc5c(-c6c7ccccc7c(-c7ccccc7)c7ccccc67)ccc6oc3c4c65)c2)cc1] 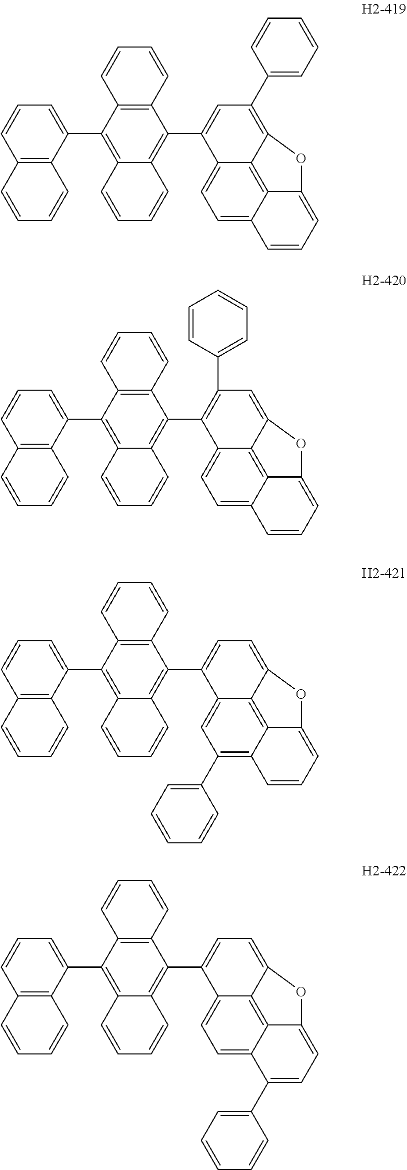 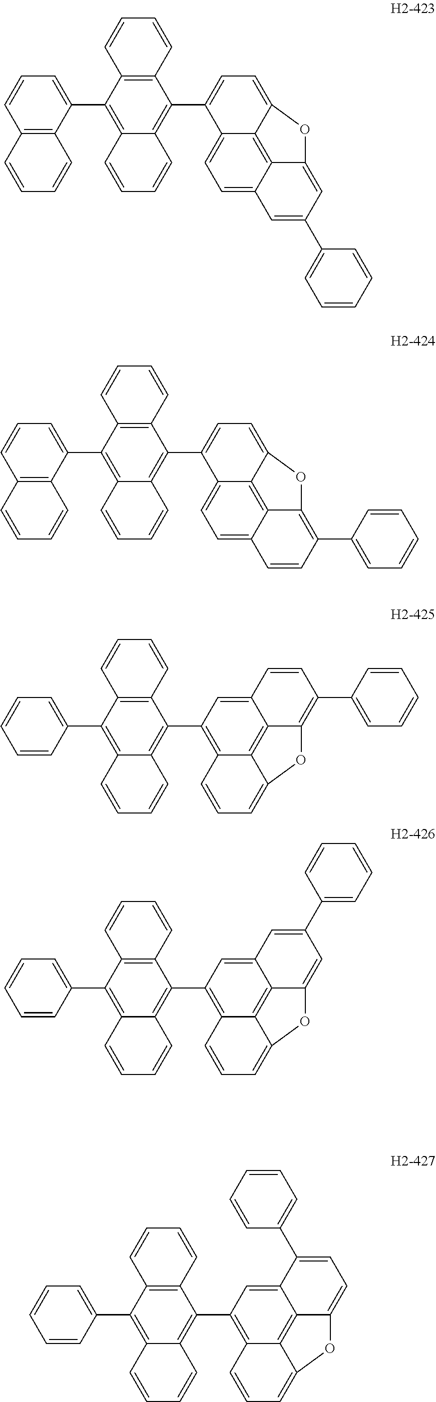 [SMILES: c1ccc(-c2cc3ccc4c(-c5c6ccccc6c(-c6cccc7ccccc67)c6ccccc56)ccc5oc(c2)c3c54)cc1 ; c1ccc(-c2ccc3ccc4c(-c5c6ccccc6c(-c6cccc7ccccc67)c6ccccc56)ccc5oc2c3c54)cc1 ; c1ccc(-c2c3ccccc3c(-c3cc4ccc(-c5ccccc5)c5oc6cccc3c6c45)c3ccccc23)cc1 ; c1ccc(-c2cc3cc(-c4c5ccccc5c(-c5ccccc5)c5ccccc45)c4cccc5oc(c2)c3c54)cc1 ; c1ccc(-c2c3ccccc3c(-c3cc4c(-c5ccccc5)ccc5oc6cccc3c6c54)c3ccccc23)cc1] 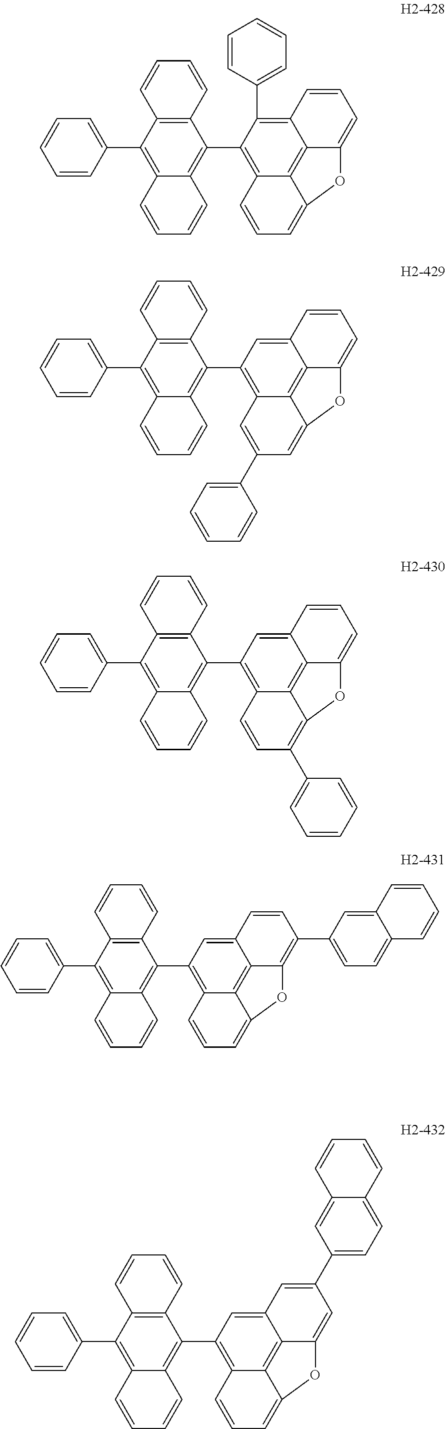 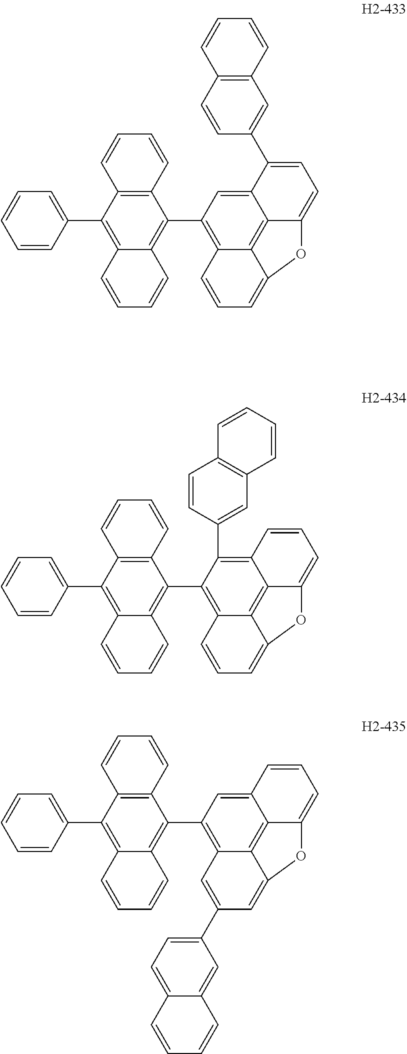 [SMILES: c1ccc(-c2c3ccccc3c(-c3cc4c(-c5ccc6ccccc6c5)ccc5oc6cccc3c6c54)c3ccccc23)cc1 ; c1ccc(-c2c3ccccc3c(-c3c(-c4ccc5ccccc5c4)c4cccc5oc6cccc3c6c54)c3ccccc23)cc1 ; c1ccc(-c2c3ccccc3c(-c3cc4cccc5oc6cc(-c7ccc8ccccc8c7)cc3c6c45)c3ccccc23)cc1] 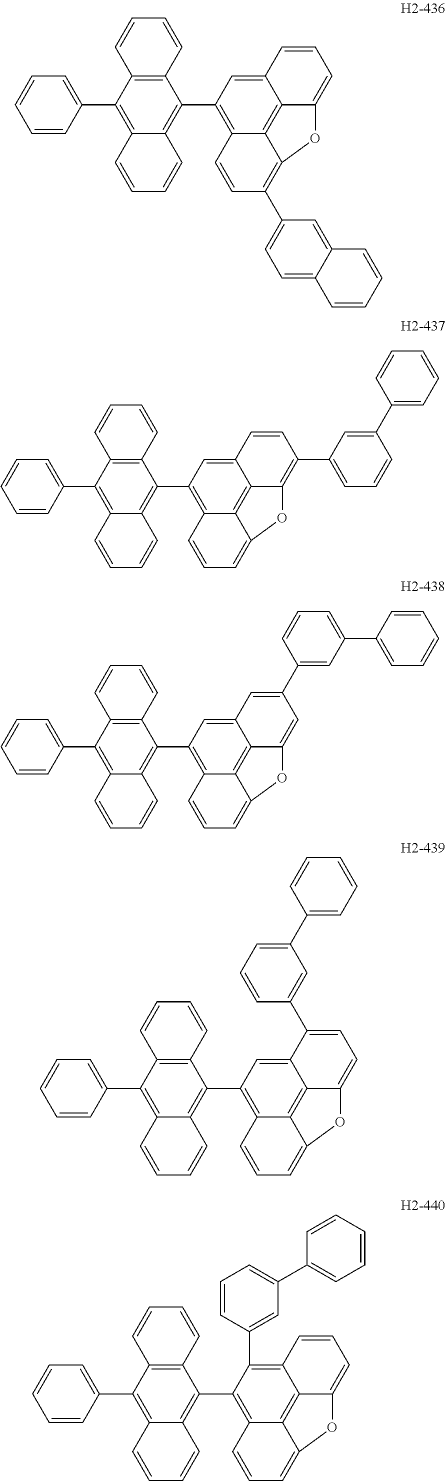 [SMILES: c1ccc(-c2c3ccccc3c(-c3cc4cccc5oc6c(-c7ccc8ccccc8c7)ccc3c6c45)c3ccccc23)cc1 ; c1ccc(-c2cccc(-c3ccc4cc(-c5c6ccccc6c(-c6ccccc6)c6ccccc56)c5cccc6oc3c4c65)c2)cc1 ; c1ccc(-c2cccc(-c3cc4cc(-c5c6ccccc6c(-c6ccccc6)c6ccccc56)c5cccc6oc(c3)c4c65)c2)cc1 ; c1ccc(-c2cccc(-c3ccc4oc5cccc6c(-c7c8ccccc8c(-c8ccccc8)c8ccccc78)cc3c4c56)c2)cc1 ; c1ccc(-c2cccc(-c3c(-c4c5ccccc5c(-c5ccccc5)c5ccccc45)c4cccc5oc6cccc3c6c54)c2)cc1] 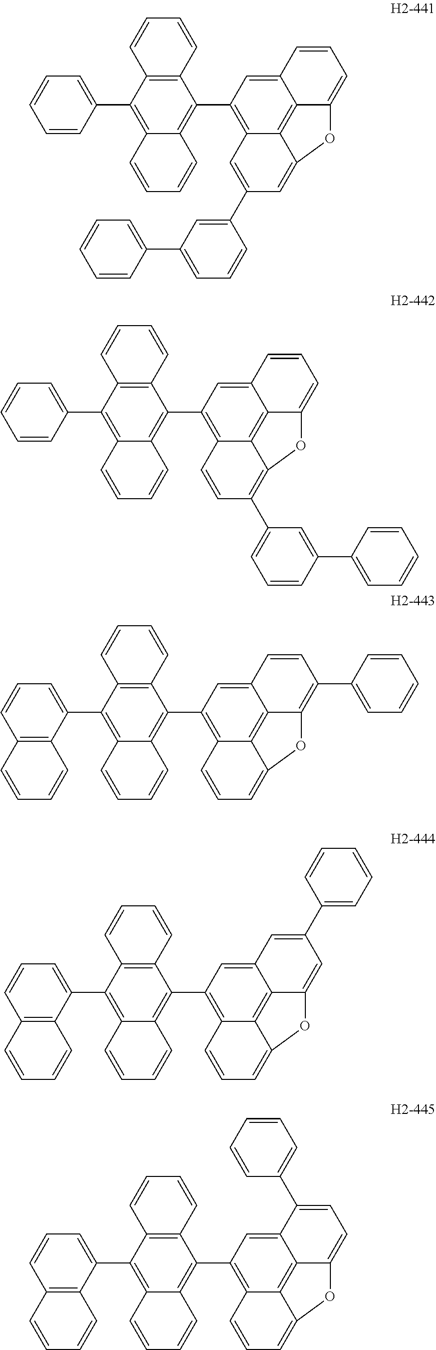 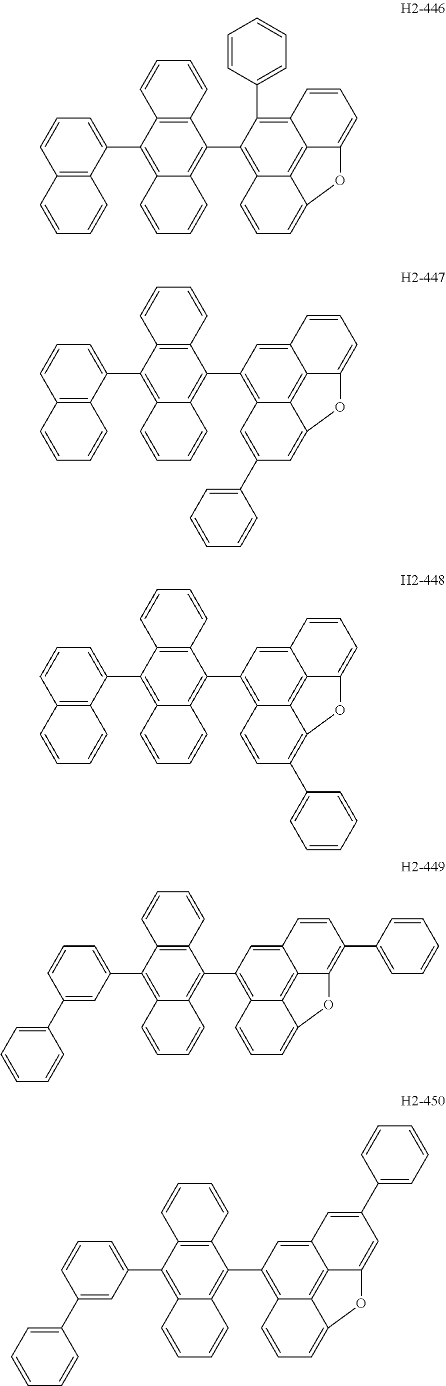 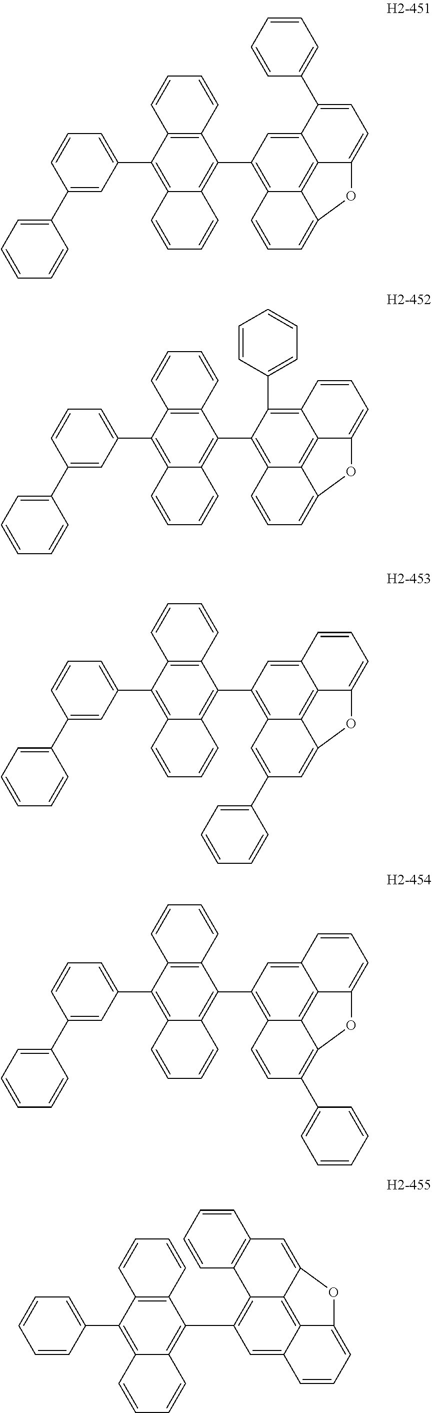 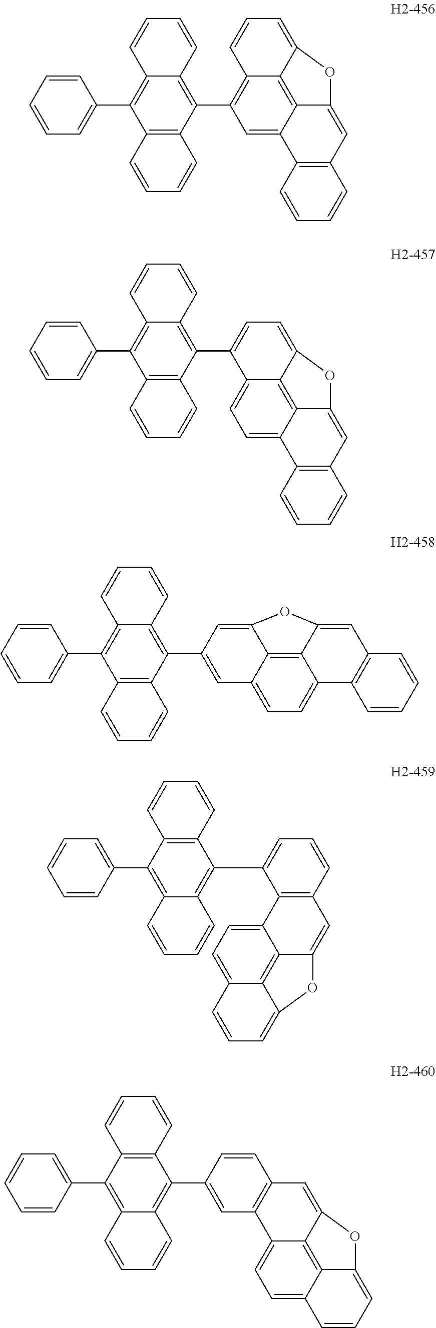 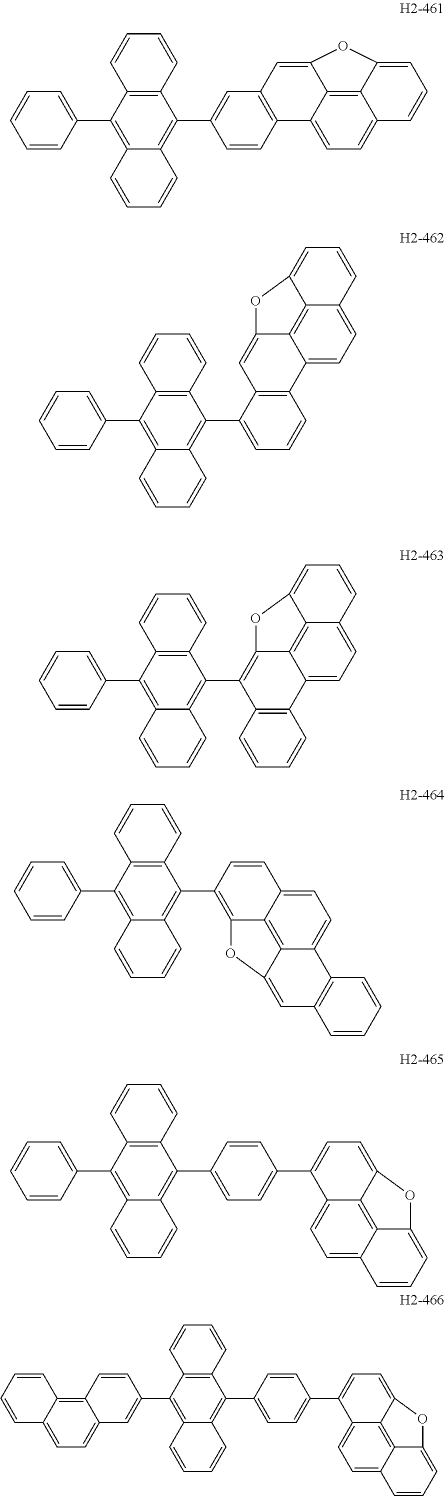 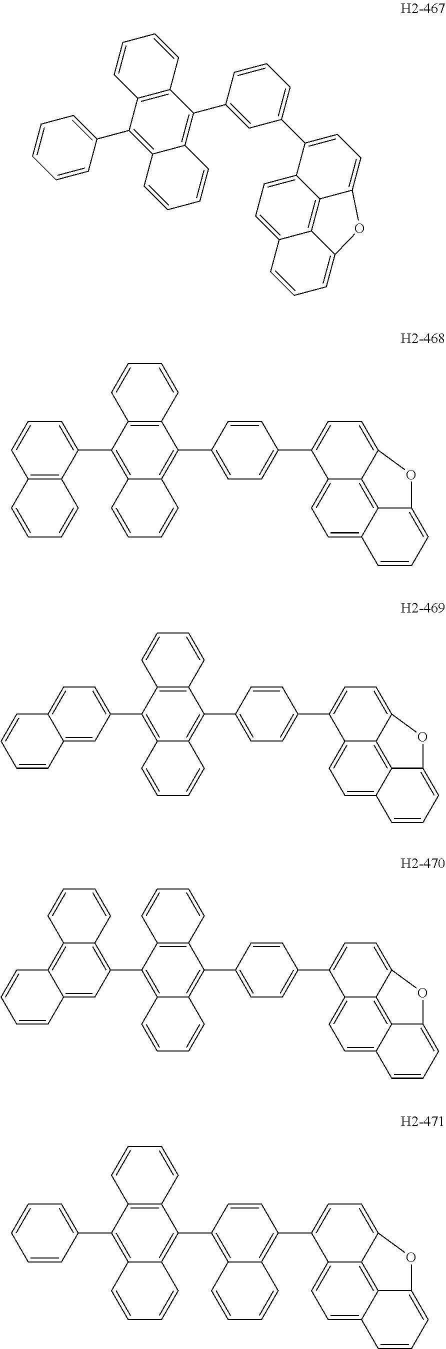 [SMILES: c1ccc(-c2c3ccccc3c(-c3cccc(-c4ccc5oc6cccc7ccc4c5c76)c3)c3ccccc23)cc1 ; c1ccc2c(-c3c4ccccc4c(-c4ccc(-c5ccc6oc7cccc8ccc5c6c87)cc4)c4ccccc34)cccc2c1 ; c1ccc2cc(-c3c4ccccc4c(-c4ccc(-c5ccc6oc7cccc8ccc5c6c87)cc4)c4ccccc34)ccc2c1 ; c1ccc2c(c1)cc(-c1c3ccccc3c(-c3ccc(-c4ccc5oc6cccc7ccc4c5c76)cc3)c3ccccc13)c1ccccc12 ; c1ccc(-c2c3ccccc3c(-c3ccc(-c4ccc5oc6cccc7ccc4c5c76)c4ccccc34)c3ccccc23)cc1] 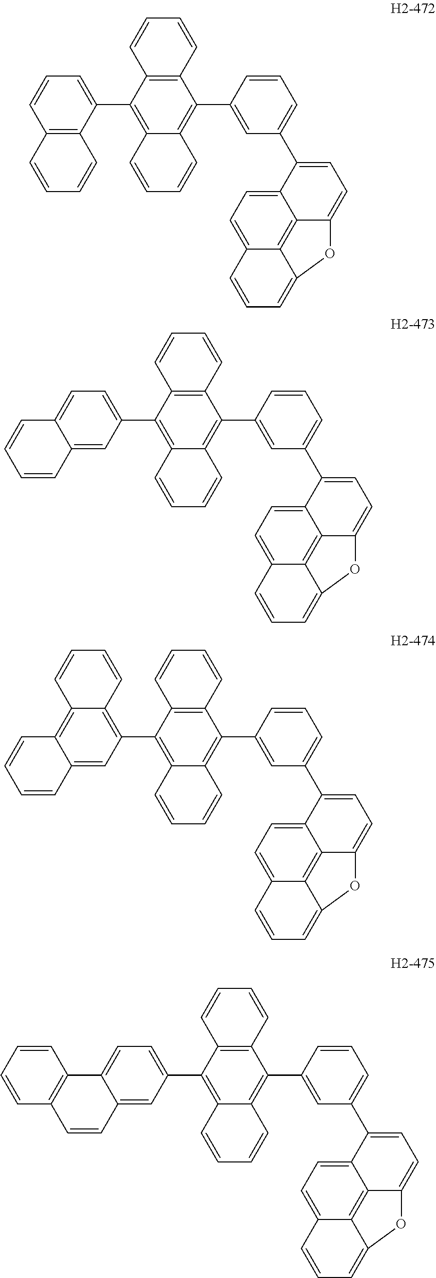 [SMILES: c1cc(-c2c3ccccc3c(-c3cccc4ccccc34)c3ccccc23)cc(-c2ccc3oc4cccc5ccc2c3c54)c1 ; c1cc(-c2c3ccccc3c(-c3ccc4ccccc4c3)c3ccccc23)cc(-c2ccc3oc4cccc5ccc2c3c54)c1 ; c1cc(-c2c3ccccc3c(-c3cc4ccccc4c4ccccc34)c3ccccc23)cc(-c2ccc3oc4cccc5ccc2c3c54)c1 ; c1cc(-c2c3ccccc3c(-c3ccc4c(ccc5ccccc54)c3)c3ccccc23)cc(-c2ccc3oc4cccc5ccc2c3c54)c1] 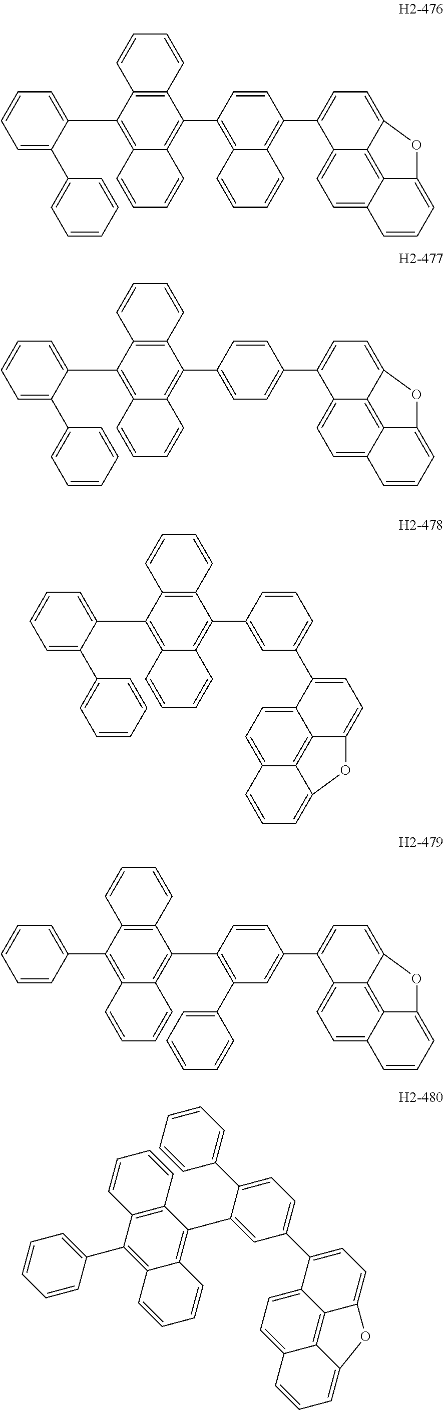 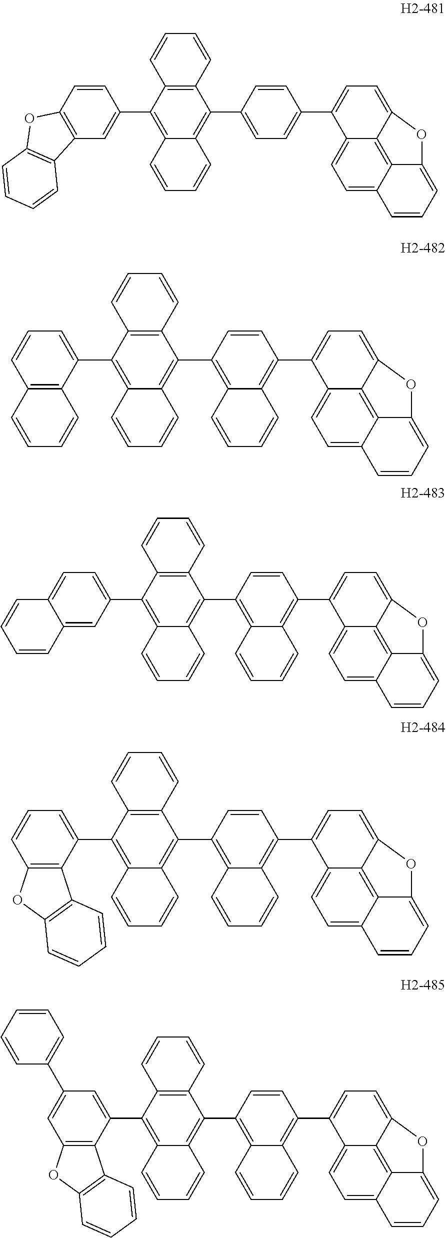 [SMILES: c1ccc2c(c1)oc1ccc(-c3c4ccccc4c(-c4ccc(-c5ccc6oc7cccc8ccc5c6c87)cc4)c4ccccc34)cc12 ; c1ccc2c(-c3c4ccccc4c(-c4ccc(-c5ccc6oc7cccc8ccc5c6c87)c5ccccc45)c4ccccc34)cccc2c1 ; c1ccc2cc(-c3c4ccccc4c(-c4ccc(-c5ccc6oc7cccc8ccc5c6c87)c5ccccc45)c4ccccc34)ccc2c1 ; c1ccc2c(c1)oc1cccc(-c3c4ccccc4c(-c4ccc(-c5ccc6oc7cccc8ccc5c6c87)c5ccccc45)c4ccccc34)c12 ; c1ccc(-c2cc(-c3c4ccccc4c(-c4ccc(-c5ccc6oc7cccc8ccc5c6c87)c5ccccc45)c4ccccc34)c3c(c2)oc2ccccc23)cc1] 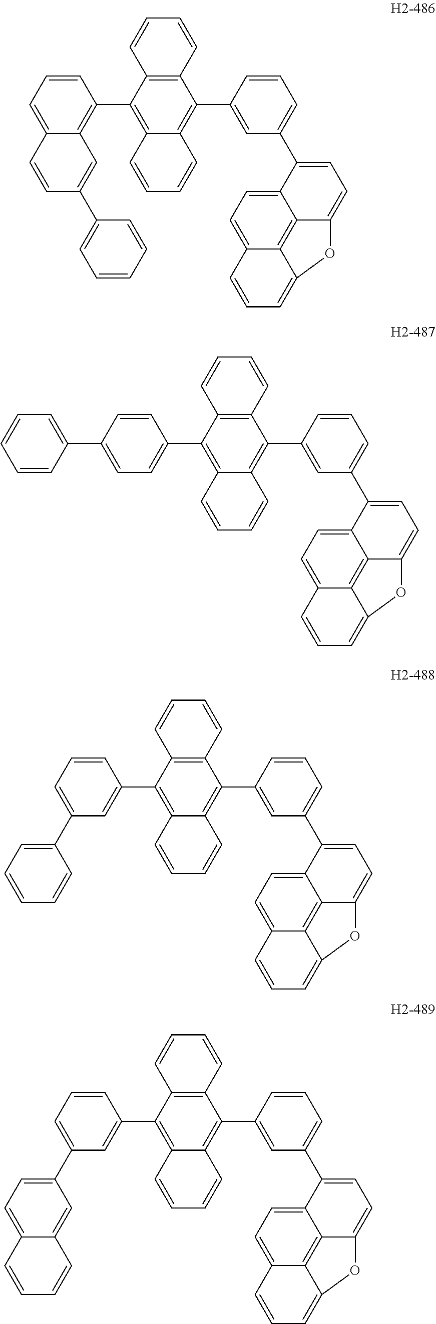 [SMILES: c1ccc(-c2ccc3cccc(-c4c5ccccc5c(-c5cccc(-c6ccc7oc8cccc9ccc6c7c98)c5)c5ccccc45)c3c2)cc1 ; c1ccc(-c2ccc(-c3c4ccccc4c(-c4cccc(-c5ccc6oc7cccc8ccc5c6c87)c4)c4ccccc34)cc2)cc1 ; c1ccc(-c2cccc(-c3c4ccccc4c(-c4cccc(-c5ccc6oc7cccc8ccc5c6c87)c4)c4ccccc34)c2)cc1 ; c1cc(-c2ccc3ccccc3c2)cc(-c2c3ccccc3c(-c3cccc(-c4ccc5oc6cccc7ccc4c5c76)c3)c3ccccc23)c1] 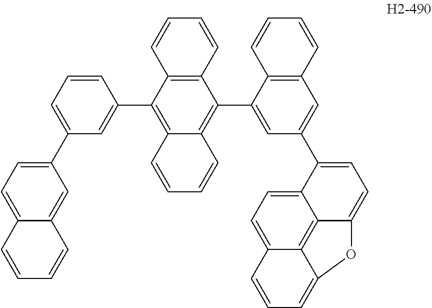 [SMILES: c1cc(-c2ccc3ccccc3c2)cc(-c2c3ccccc3c(-c3cc(-c4ccc5oc6cccc7ccc4c5c76)cc4ccccc34)c3ccccc23)c1] 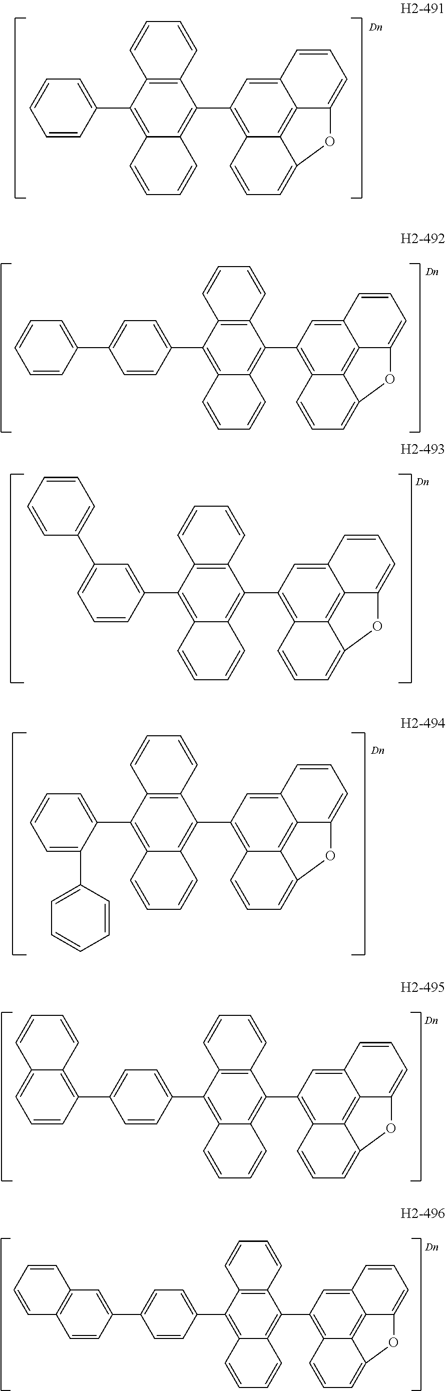 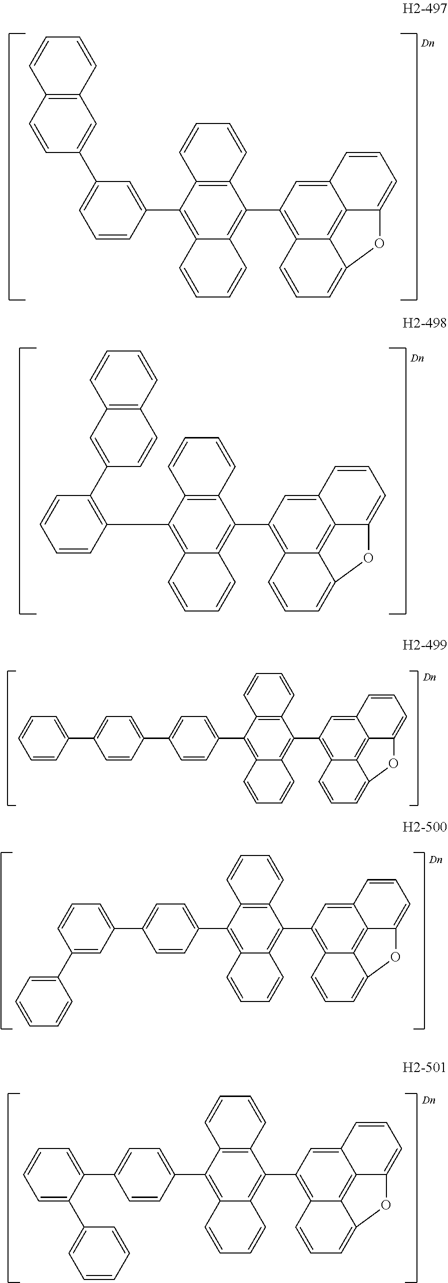 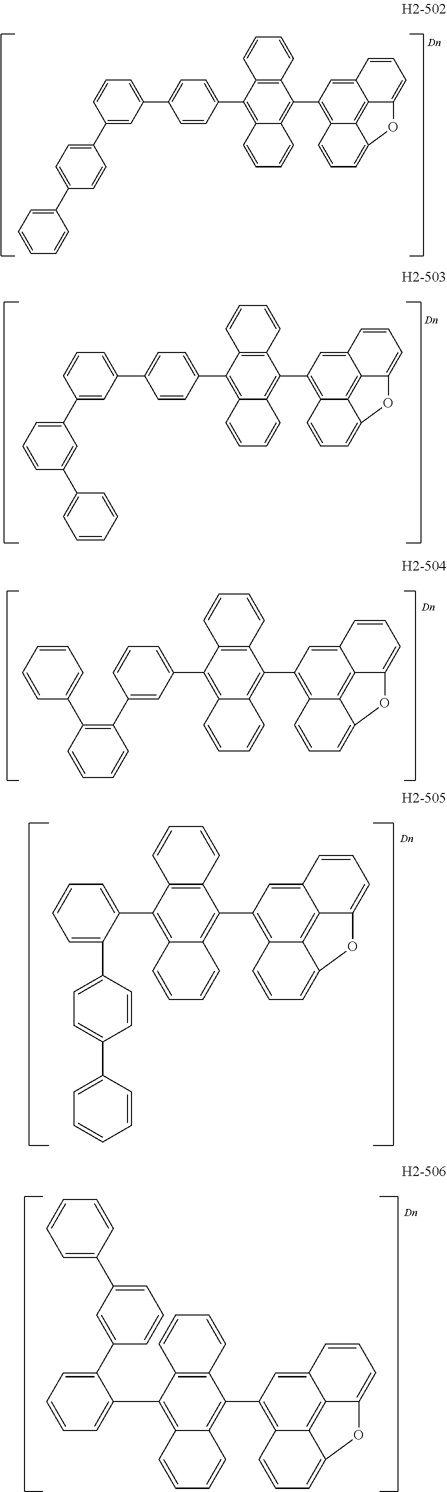 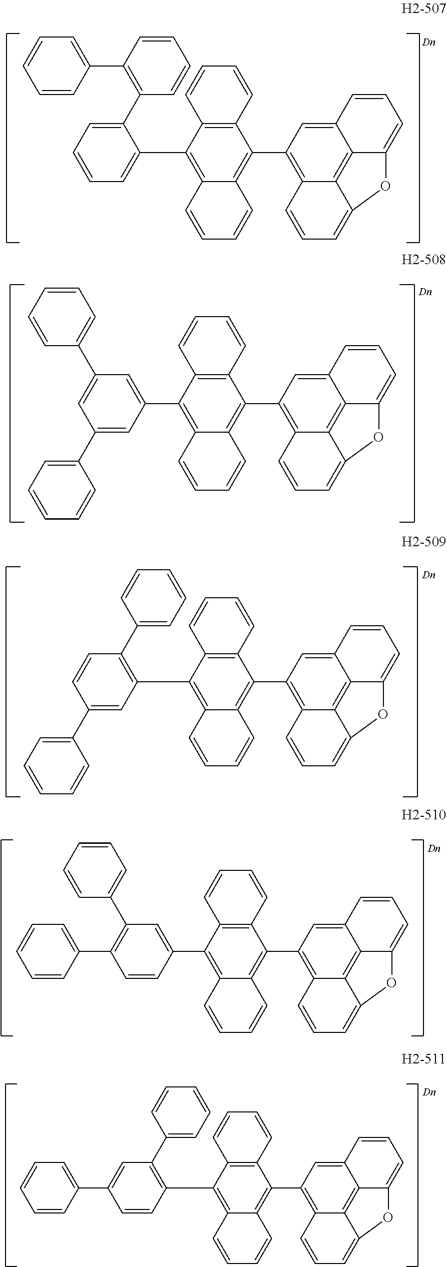 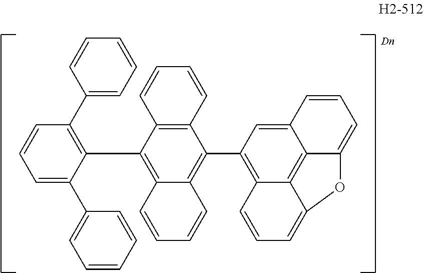 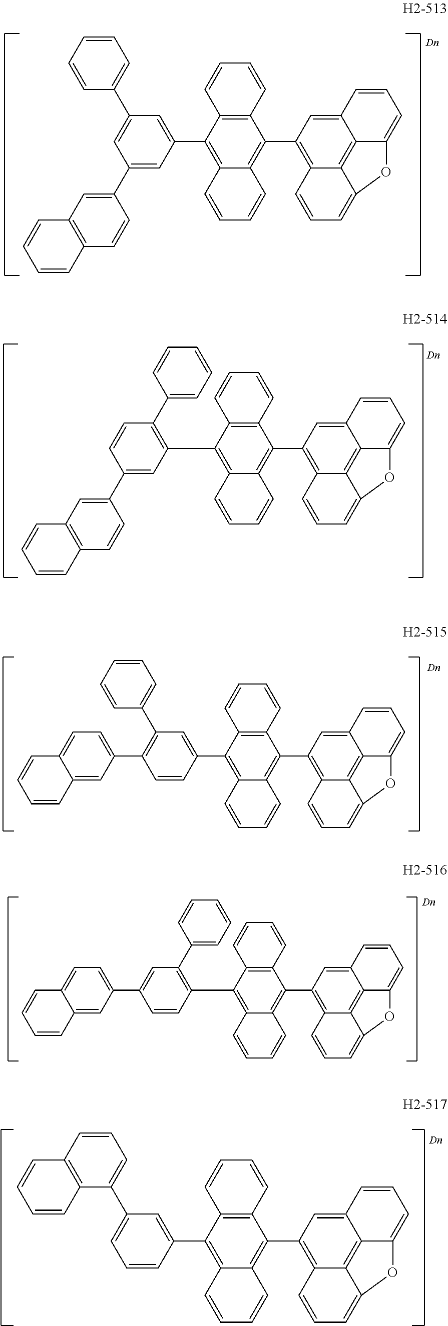 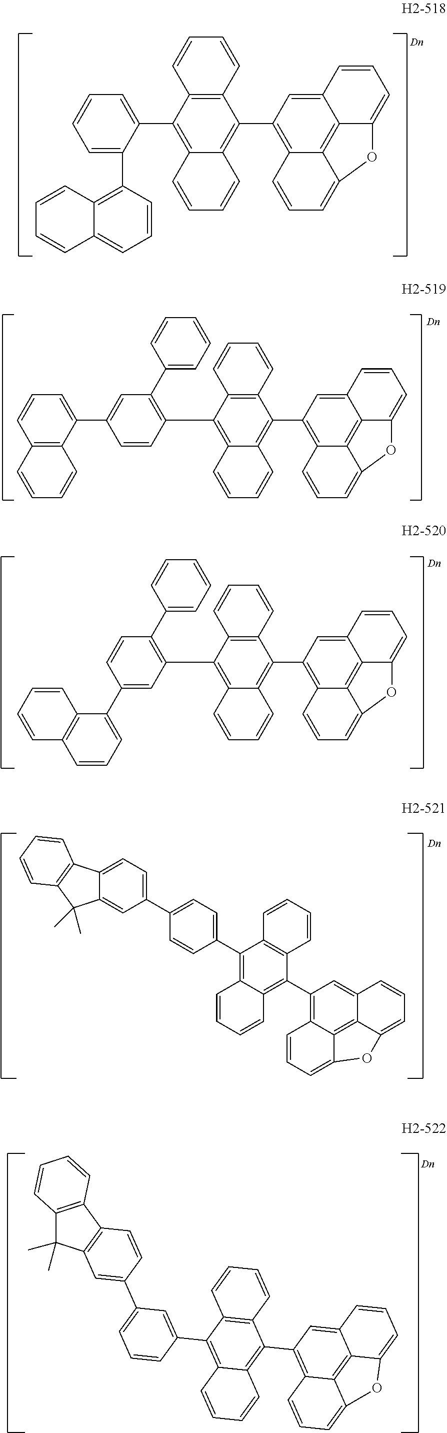 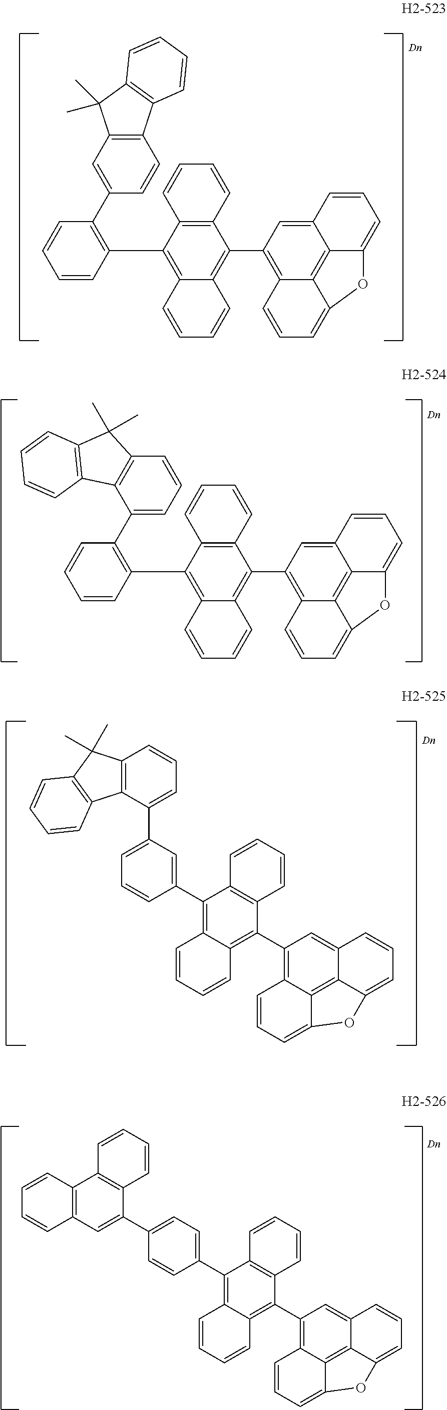 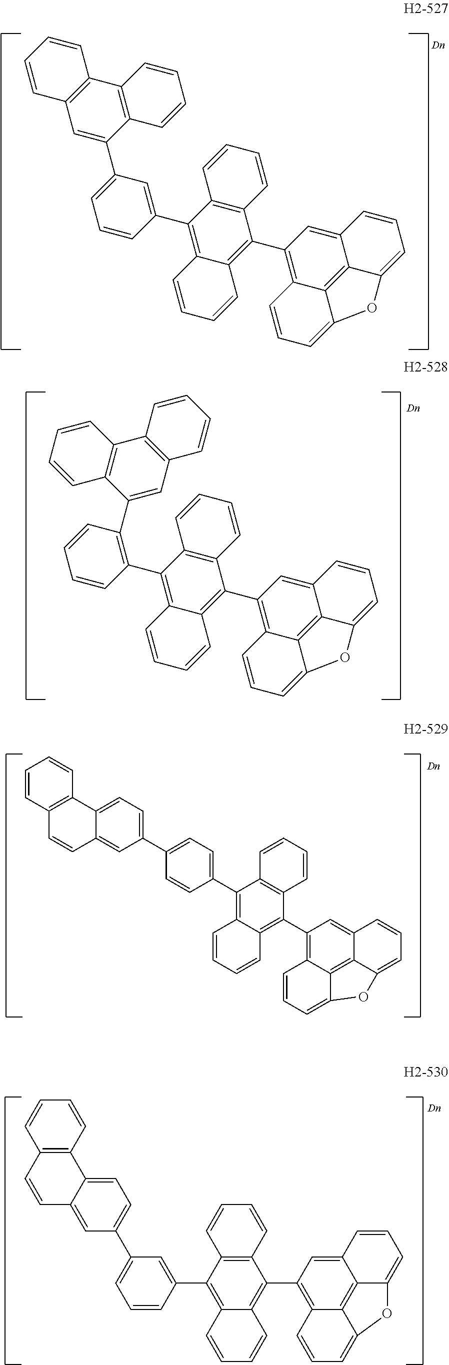 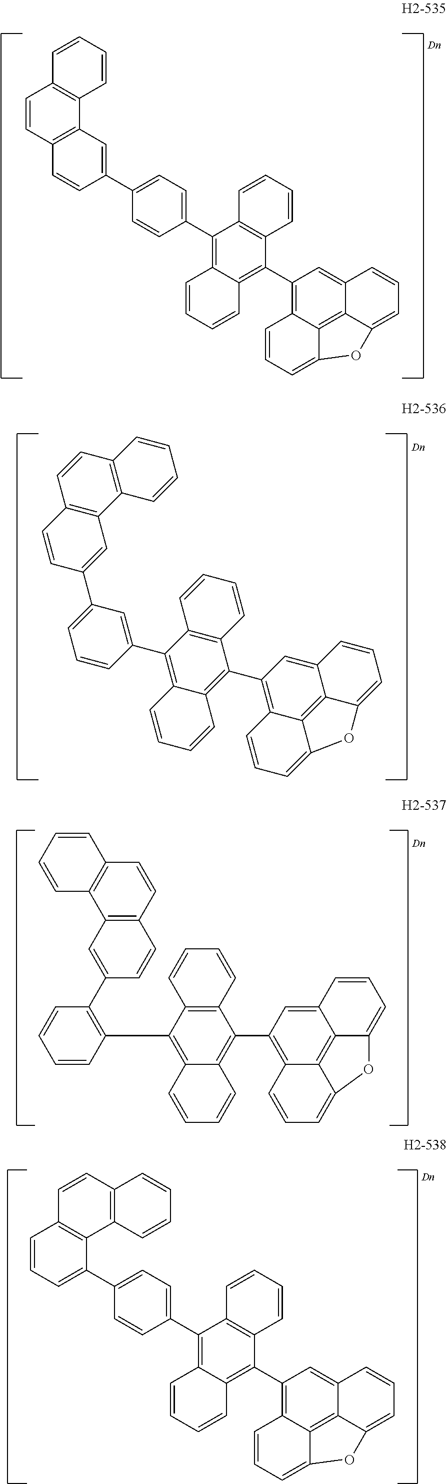 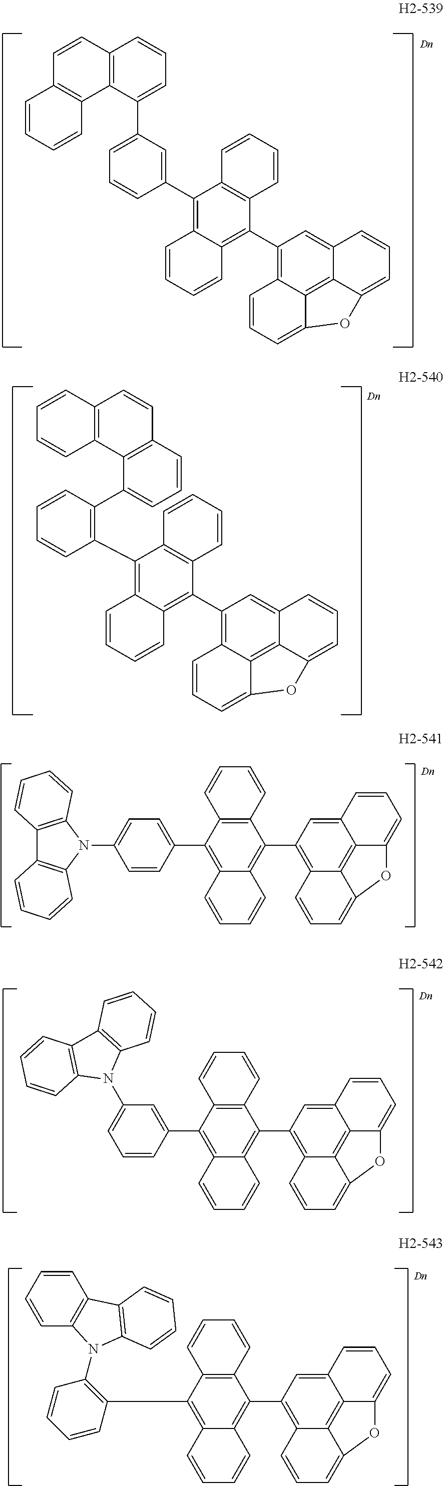 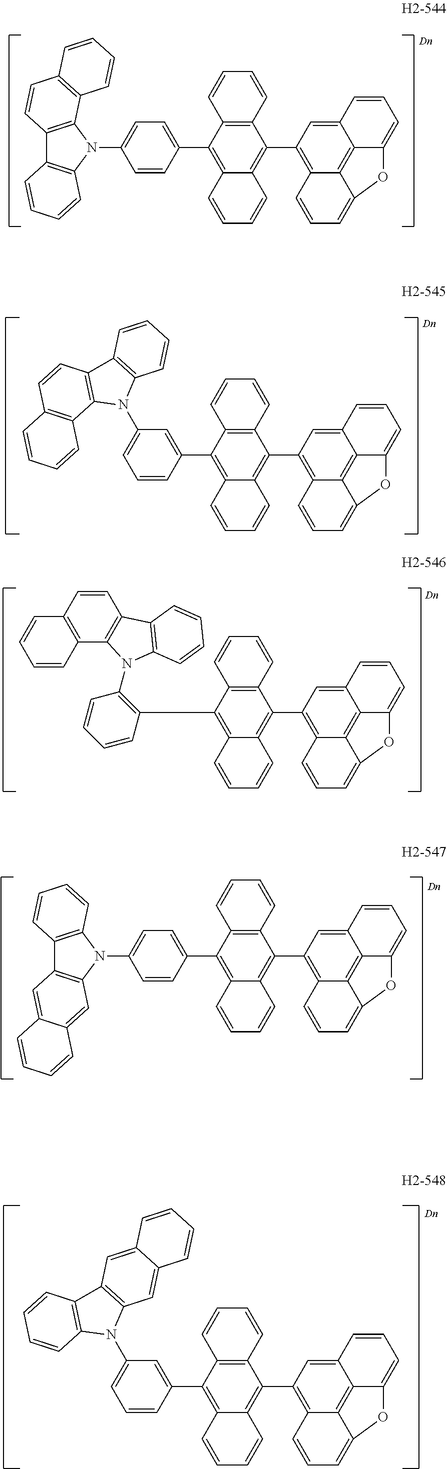 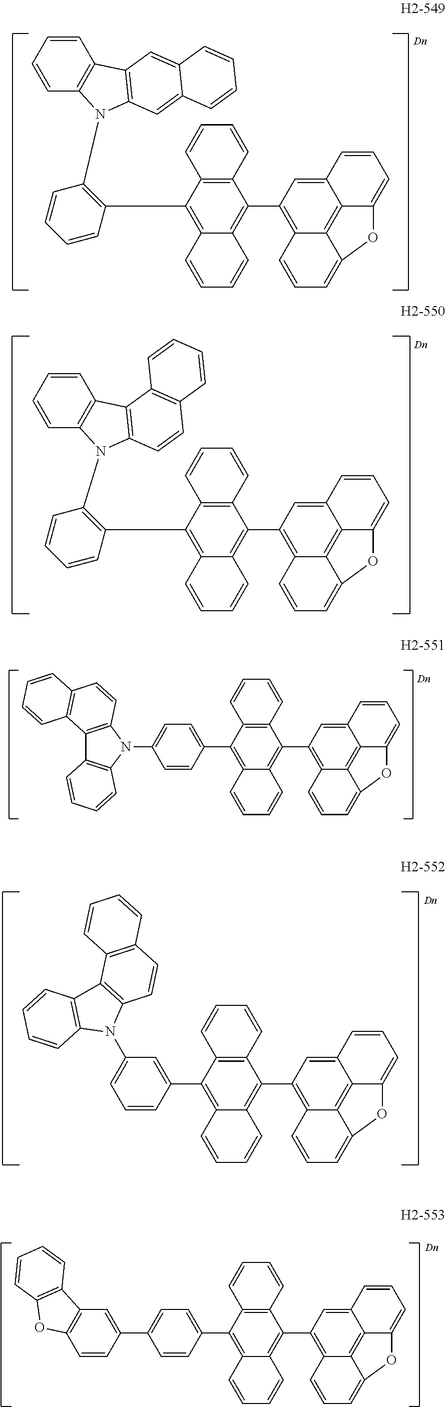 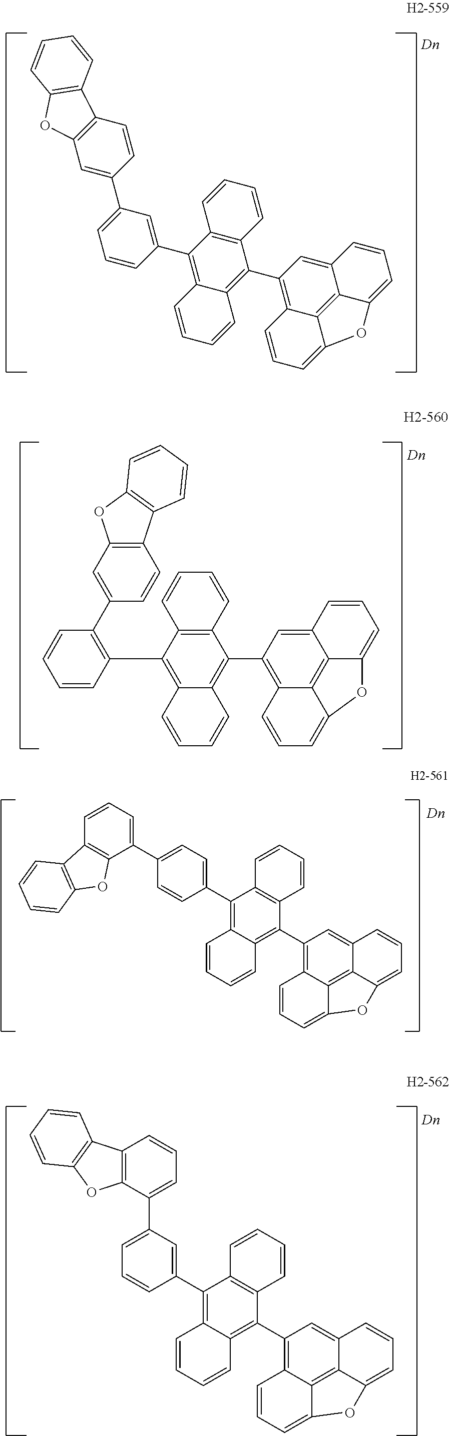 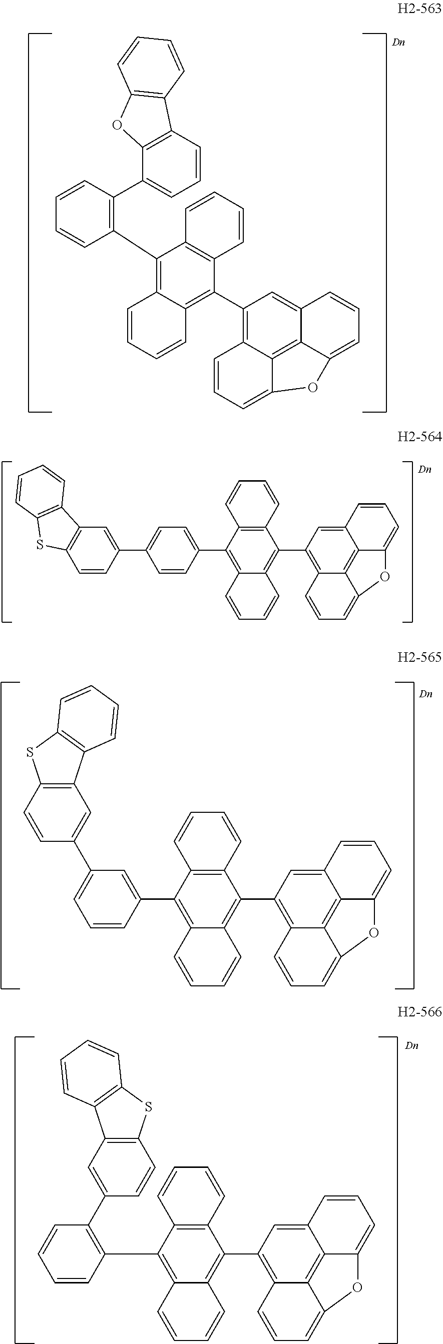 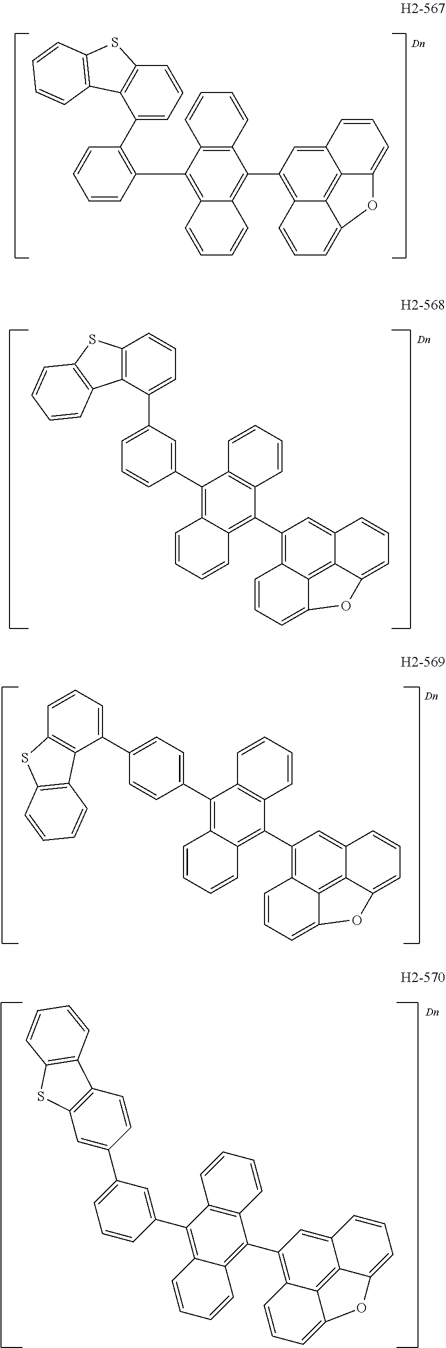 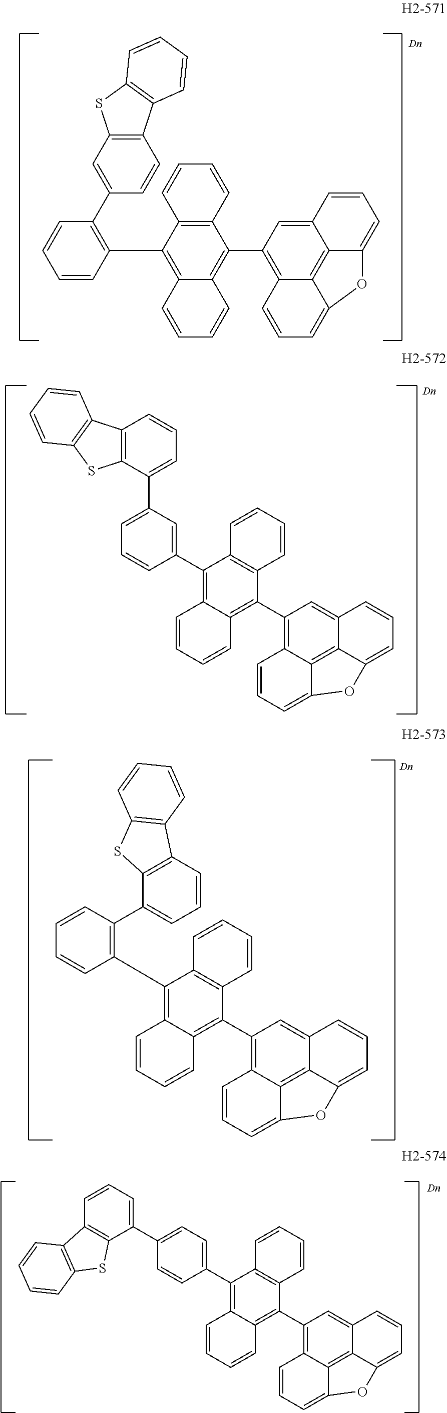 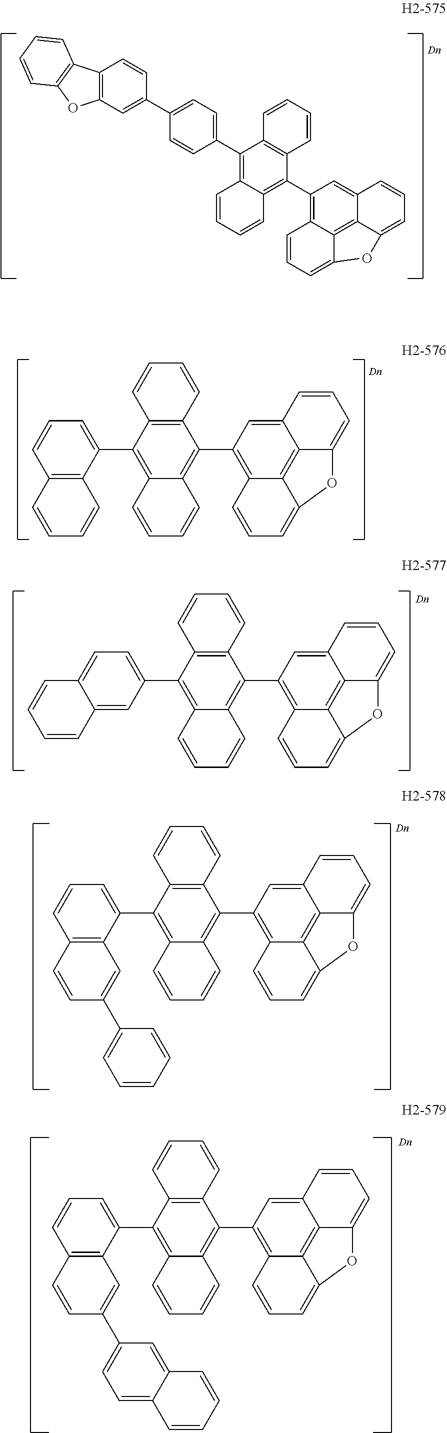 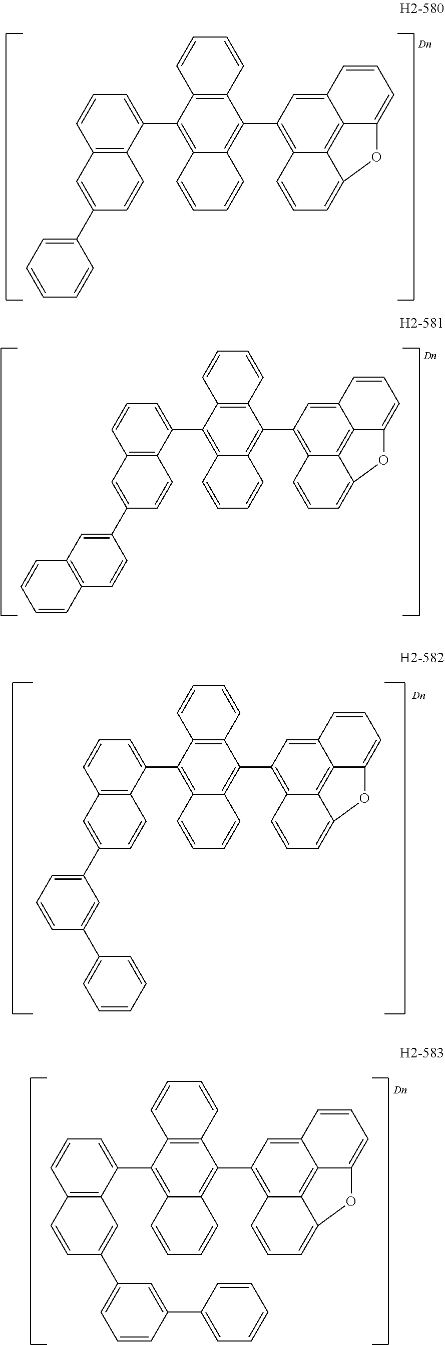 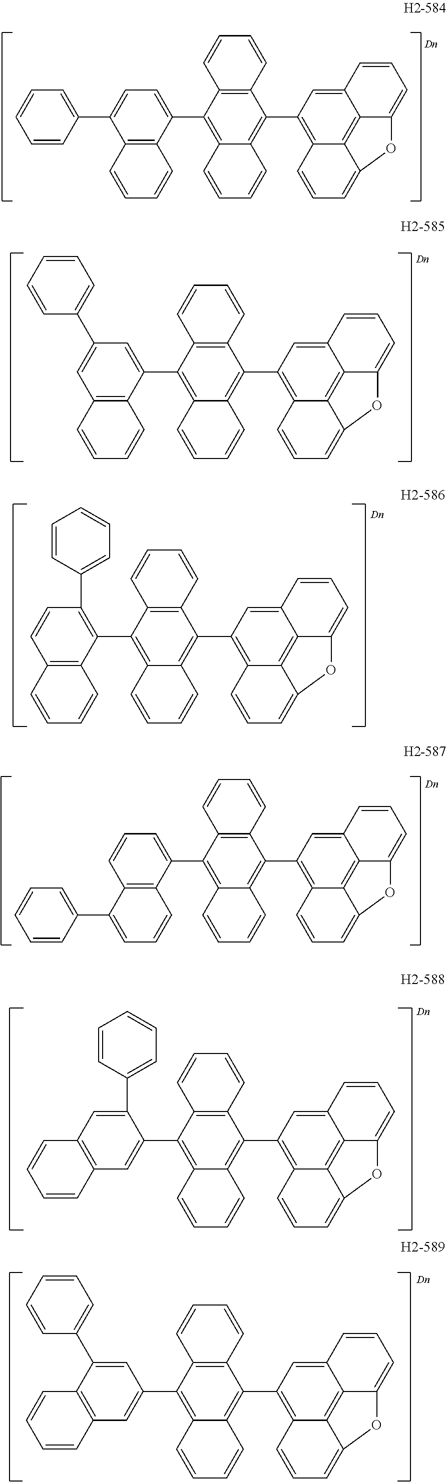 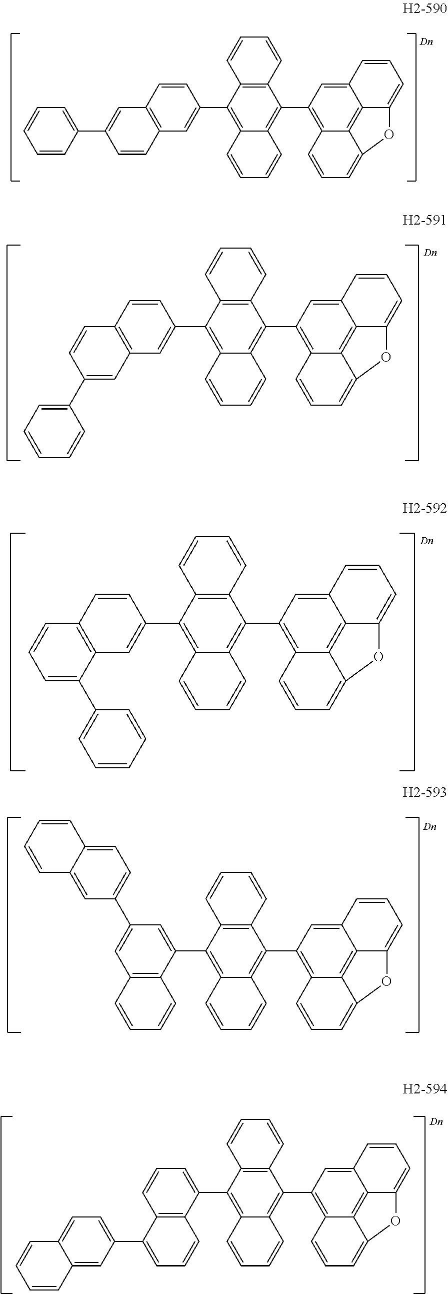 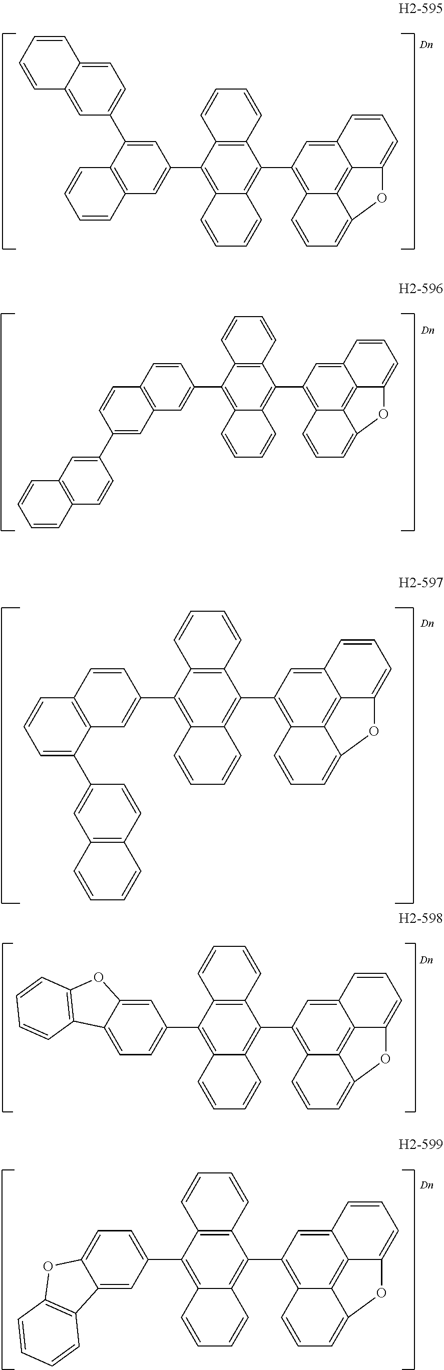 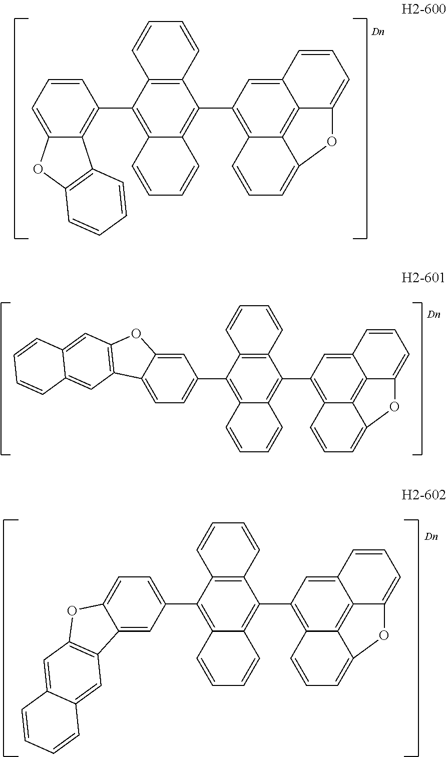 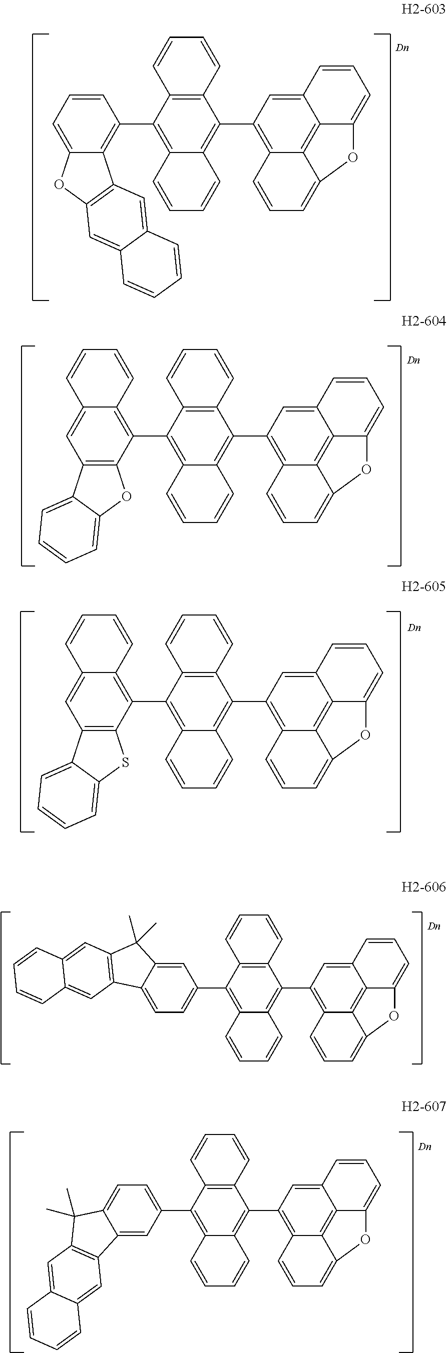 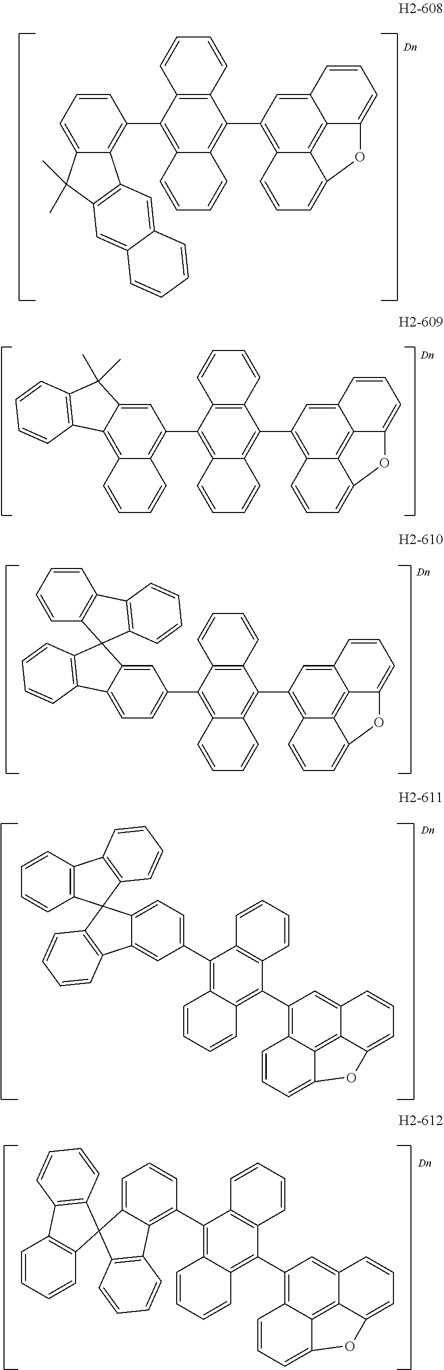 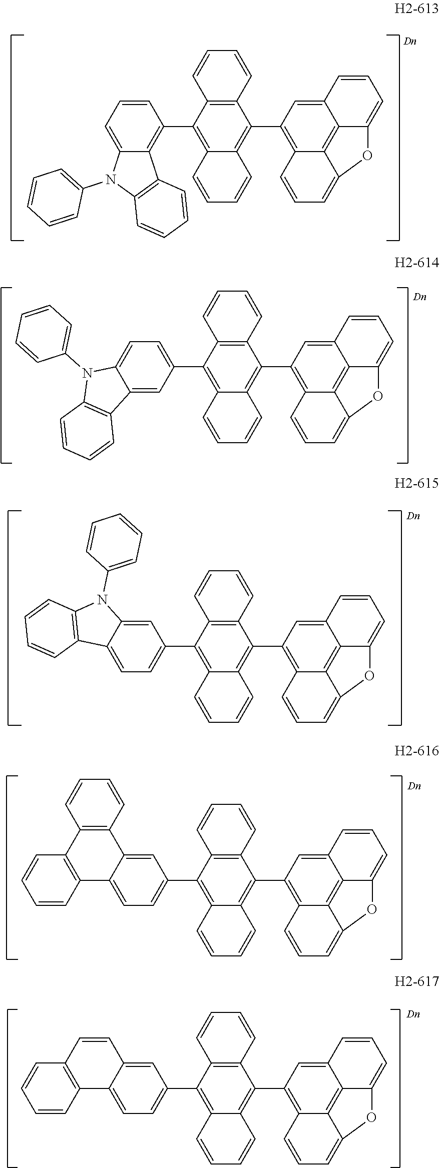 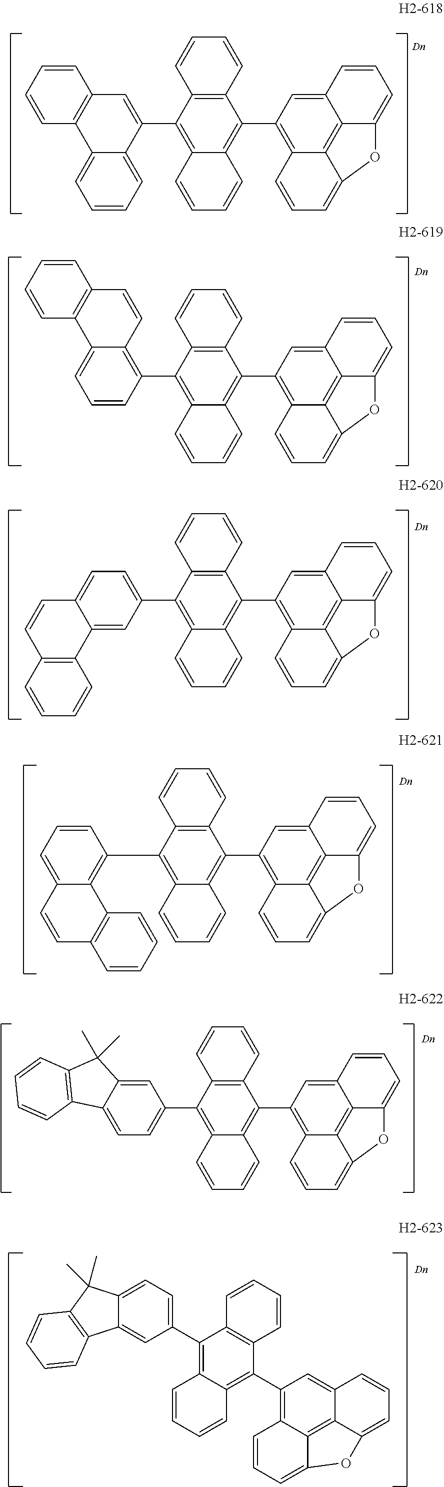 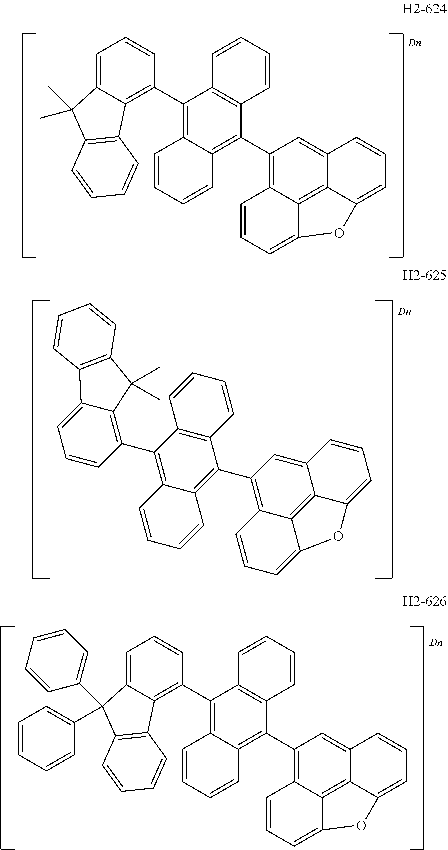 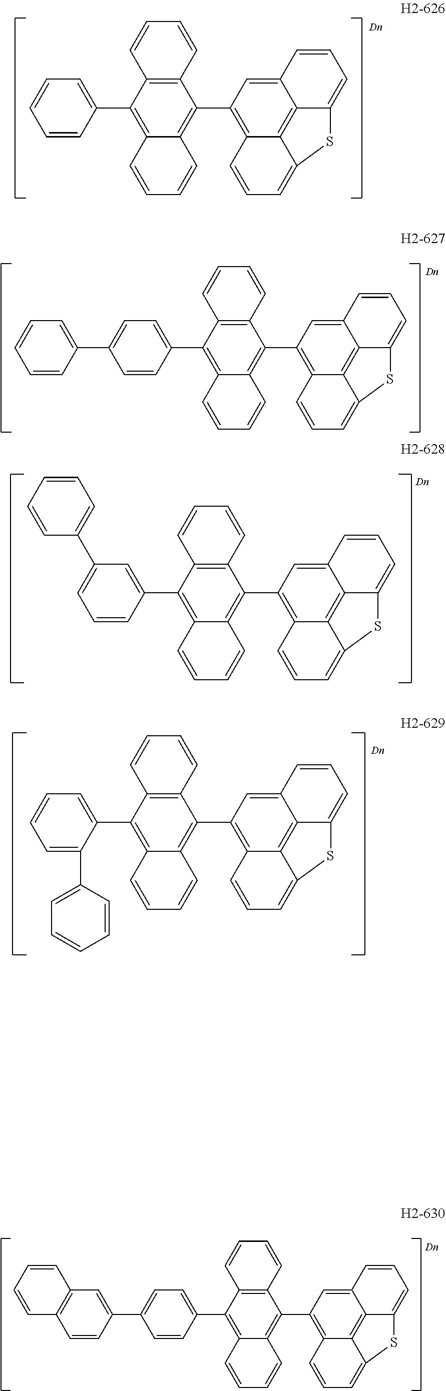 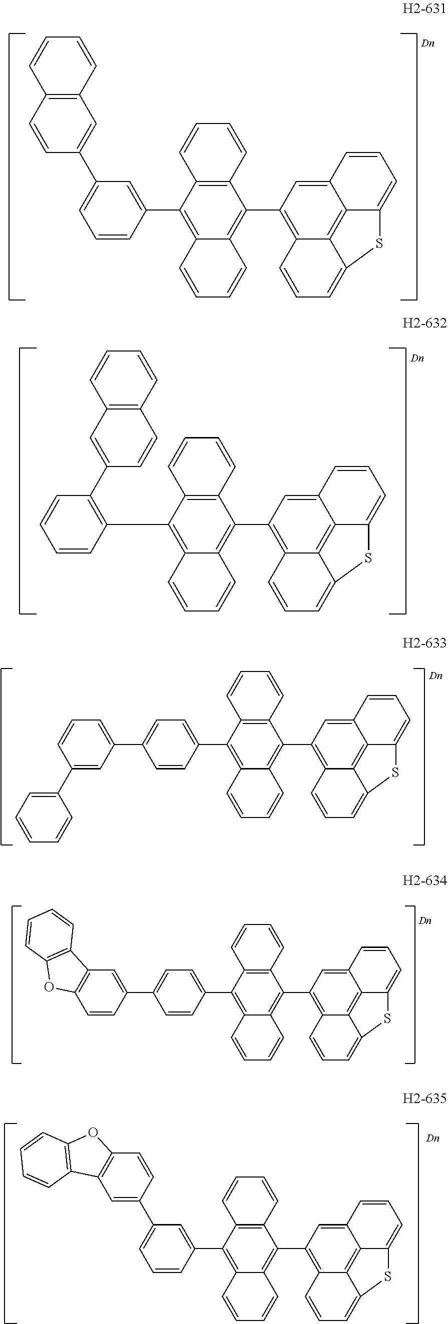 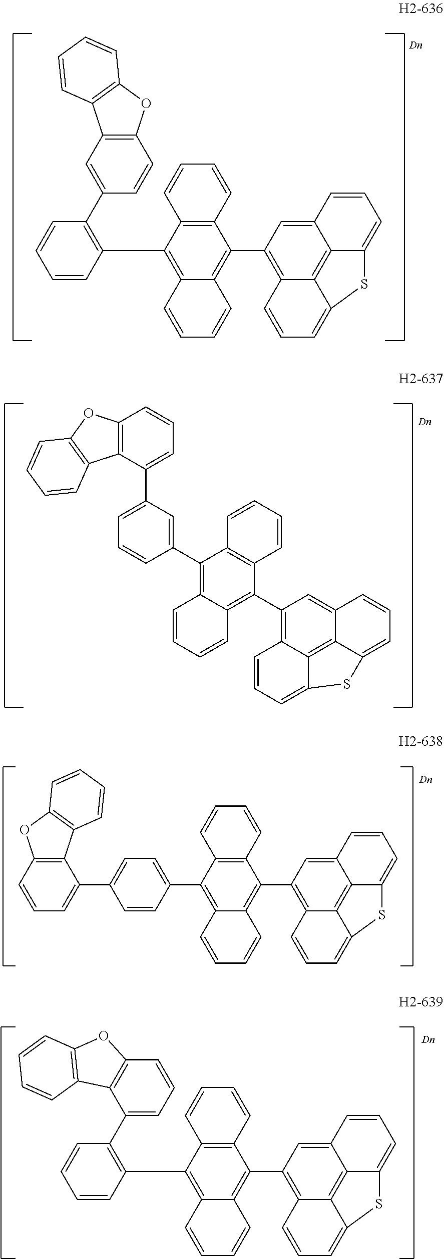 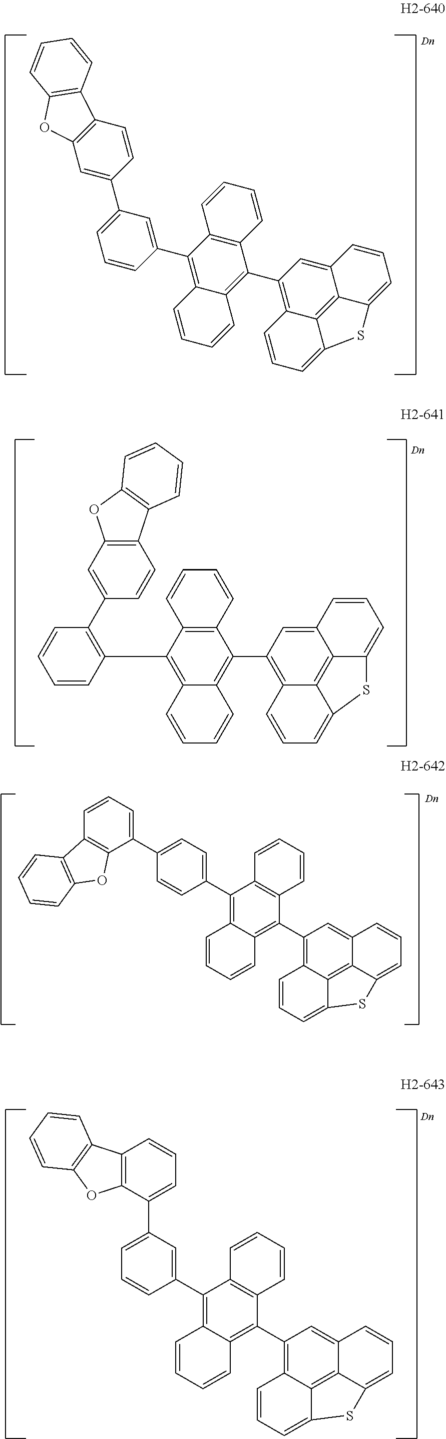 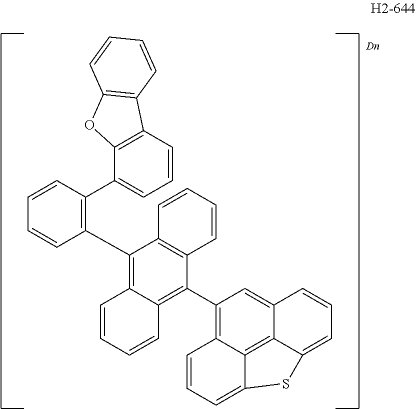 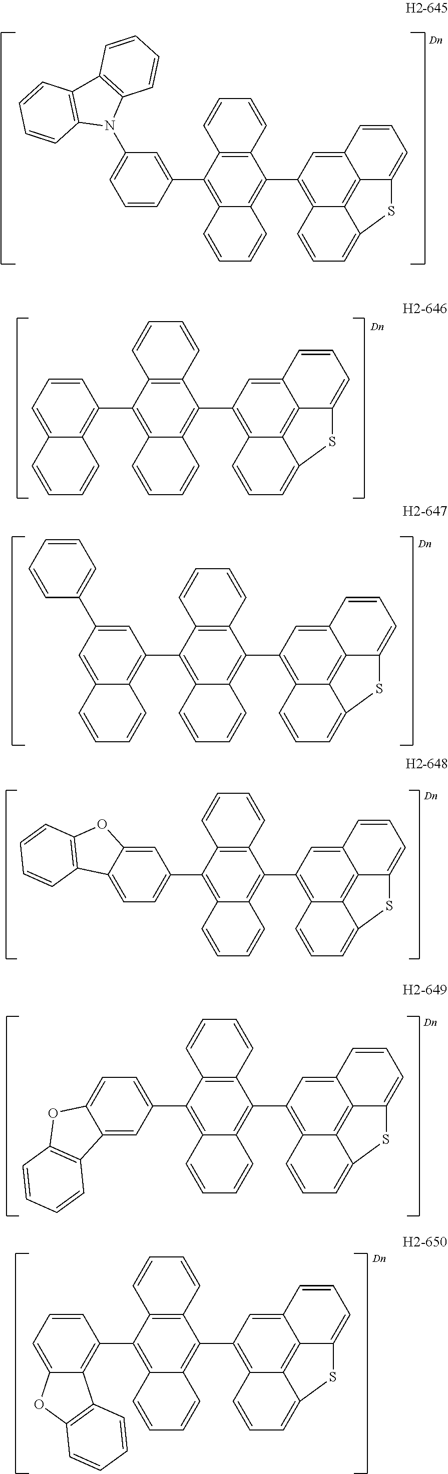 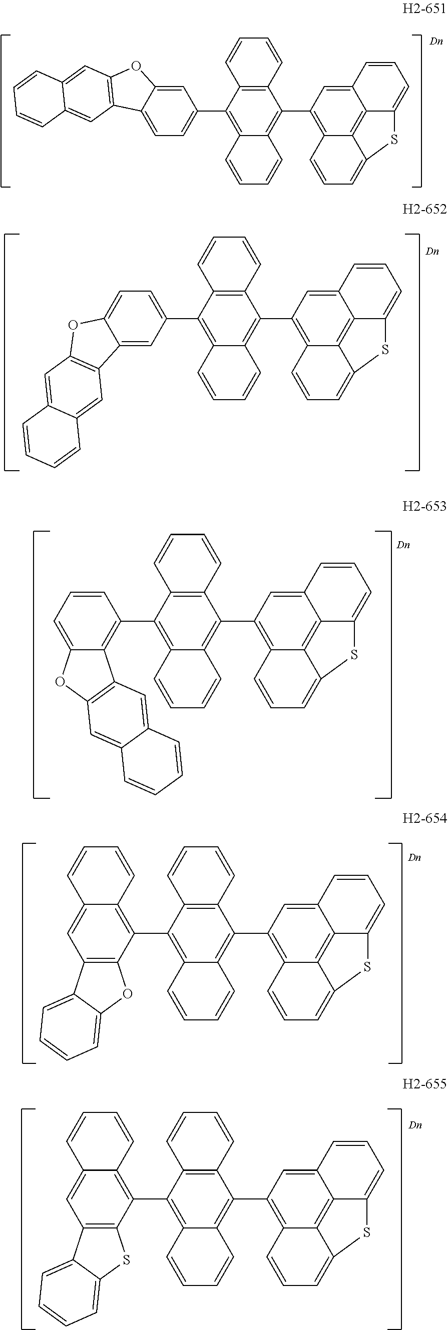 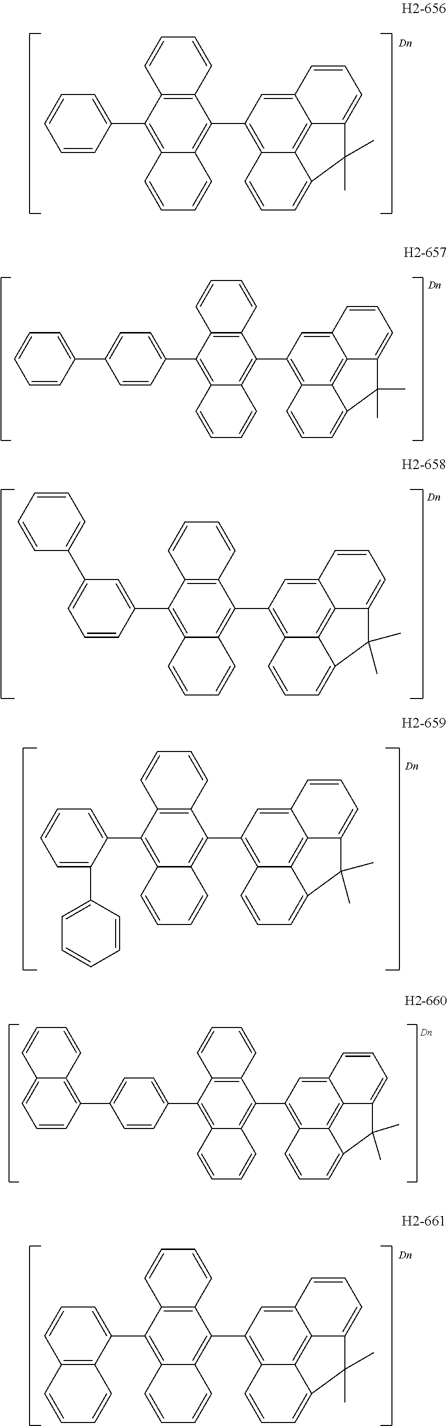 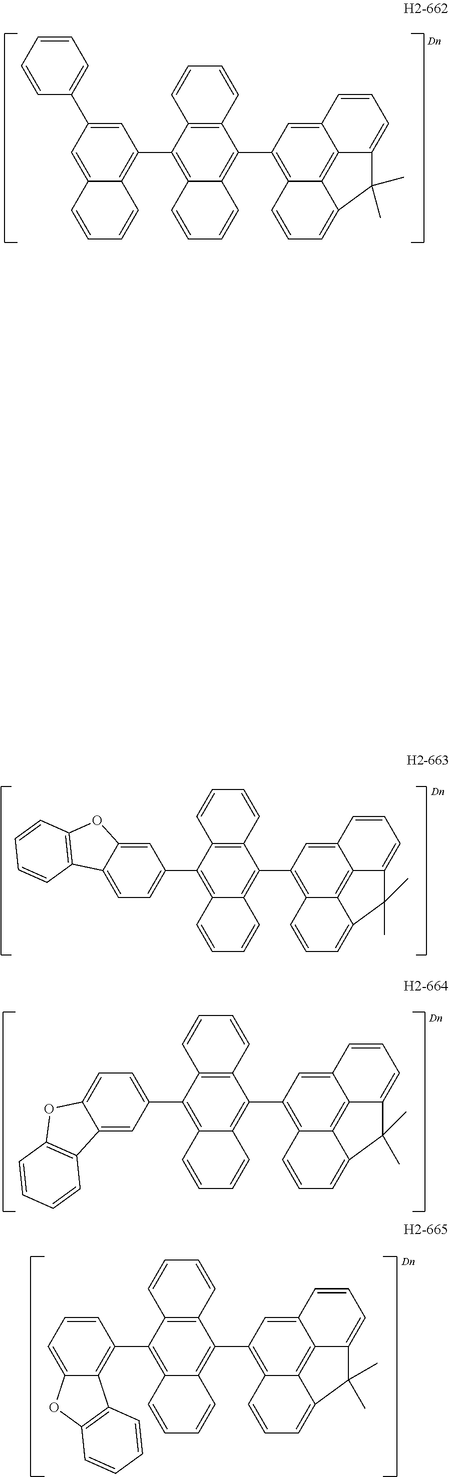 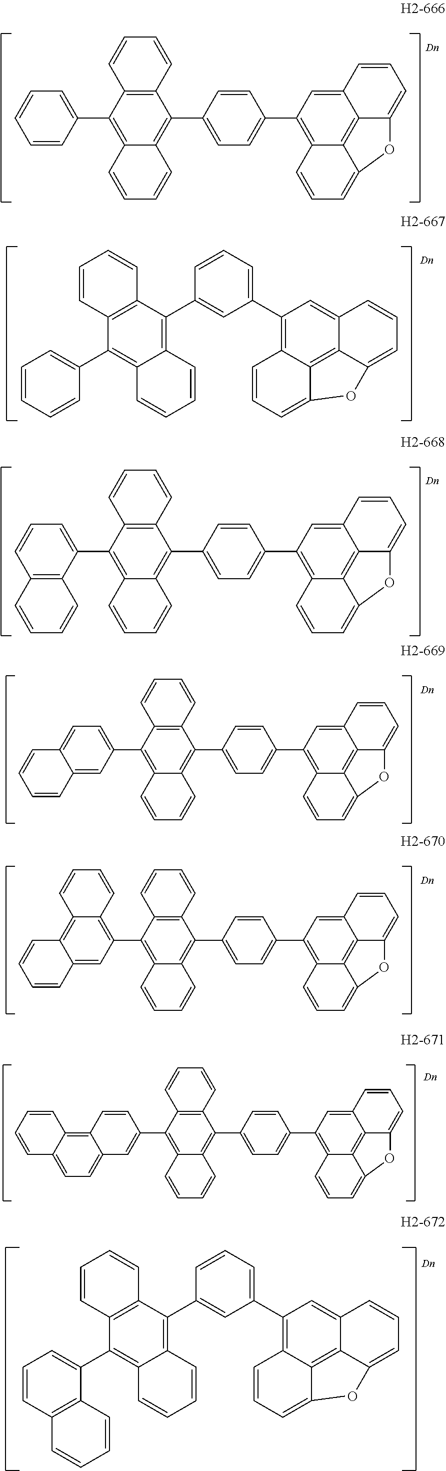 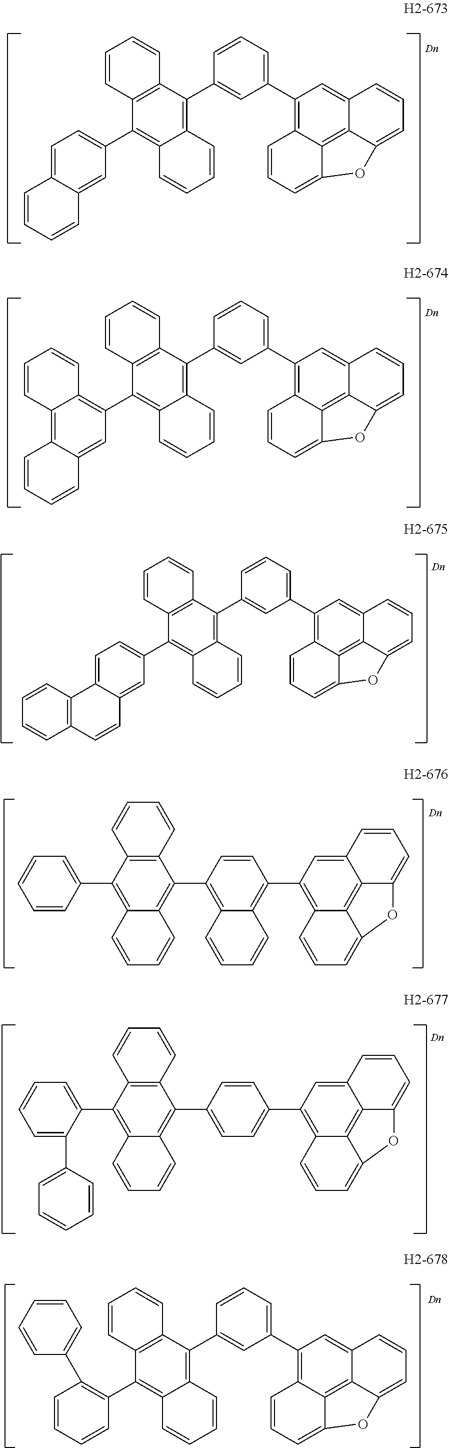 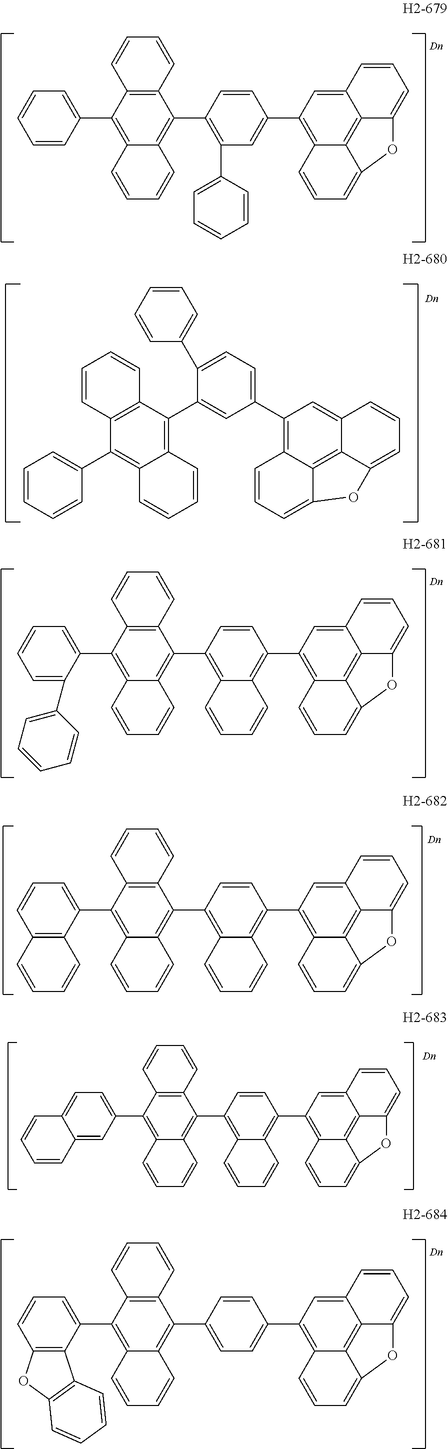 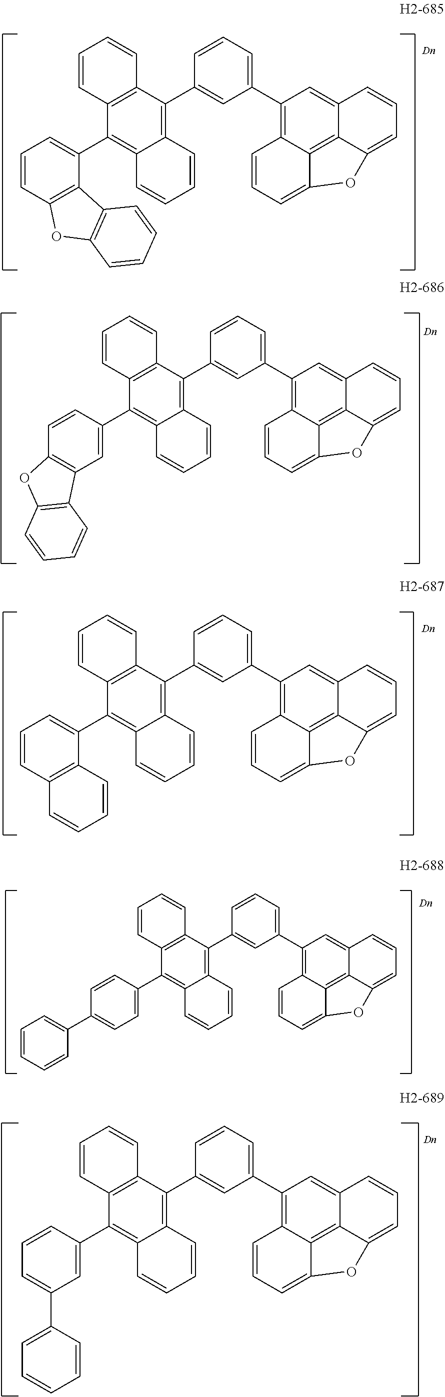 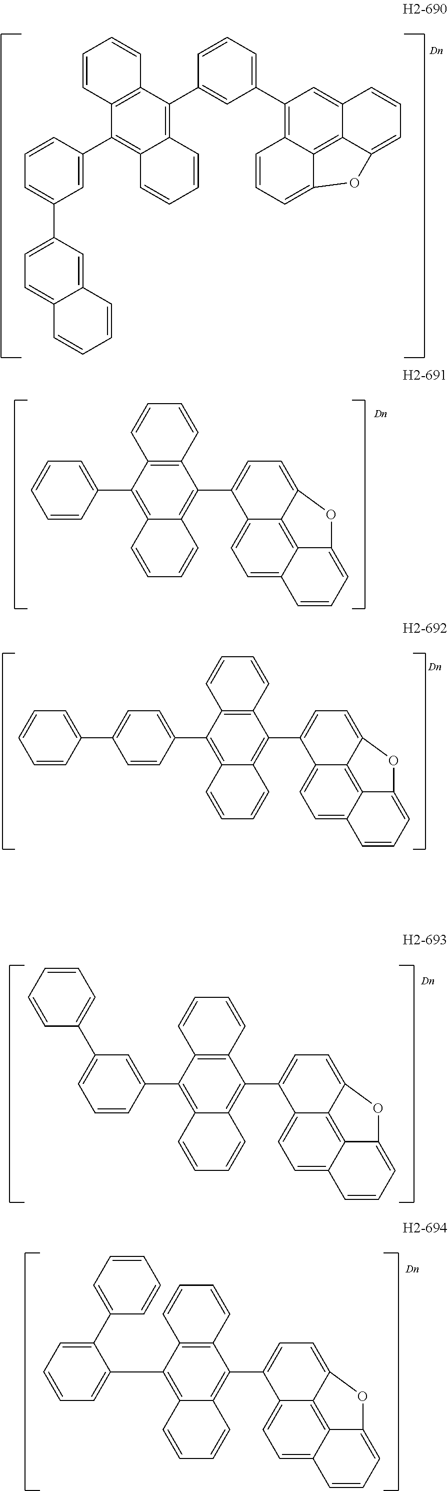 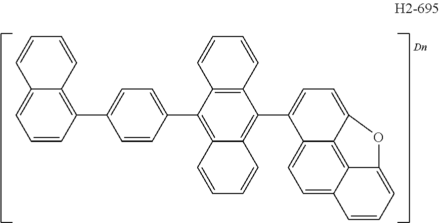 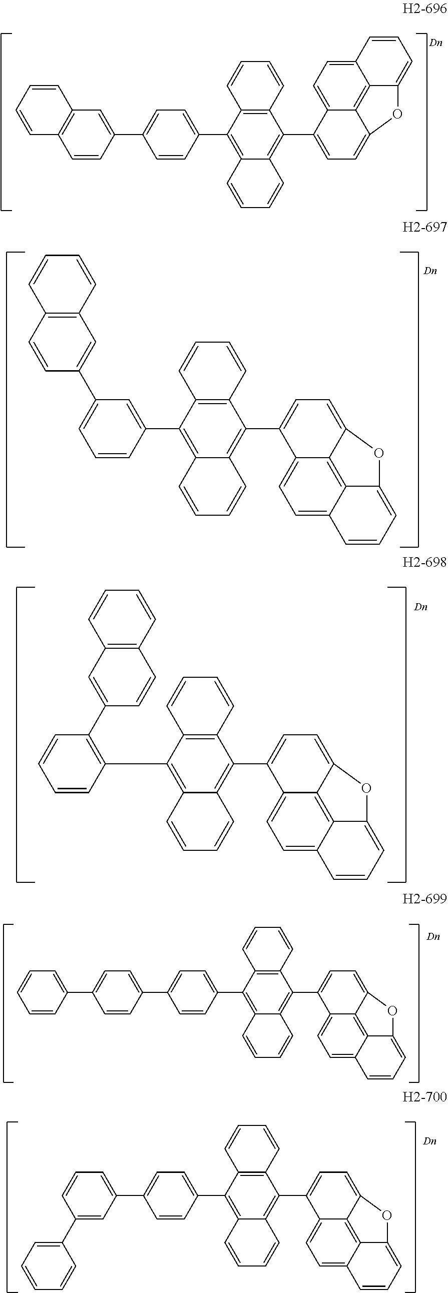 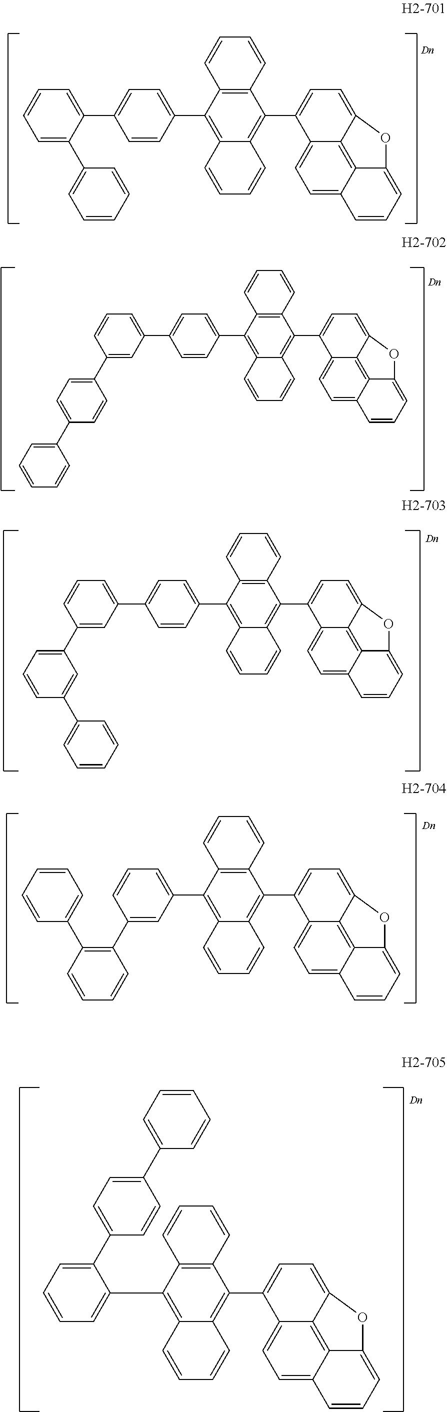 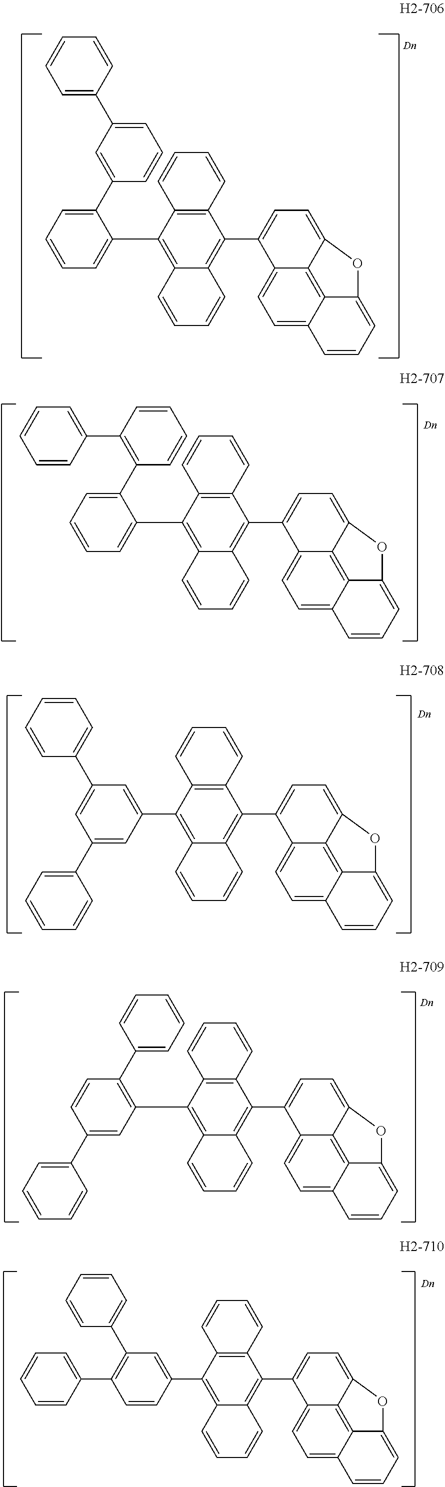 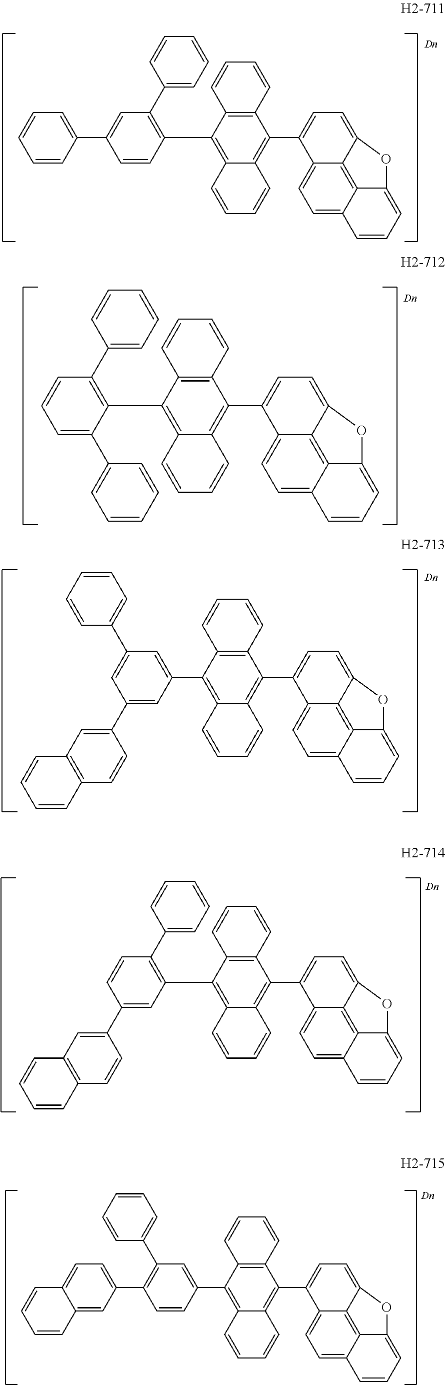 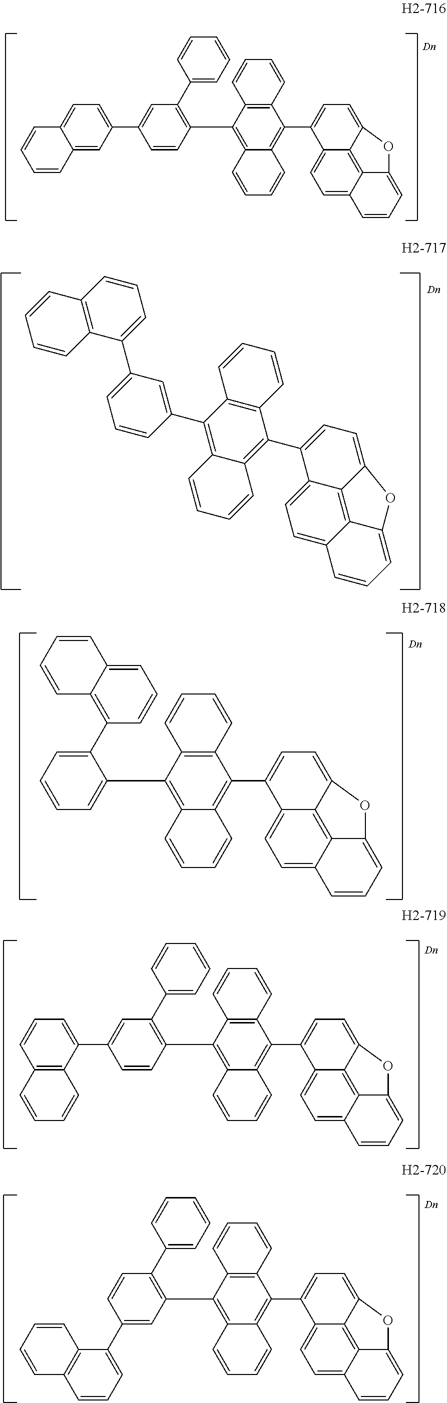 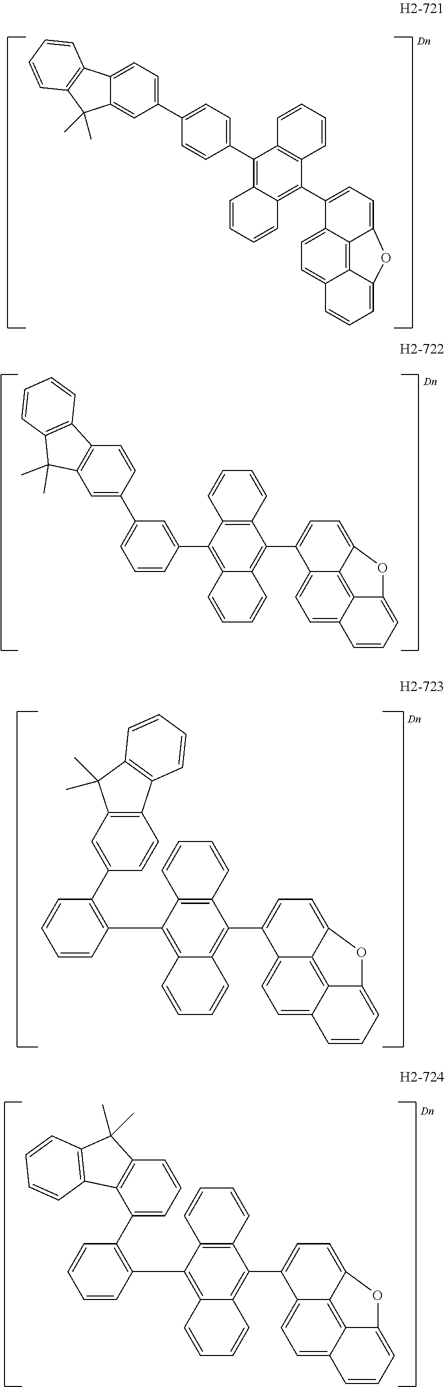 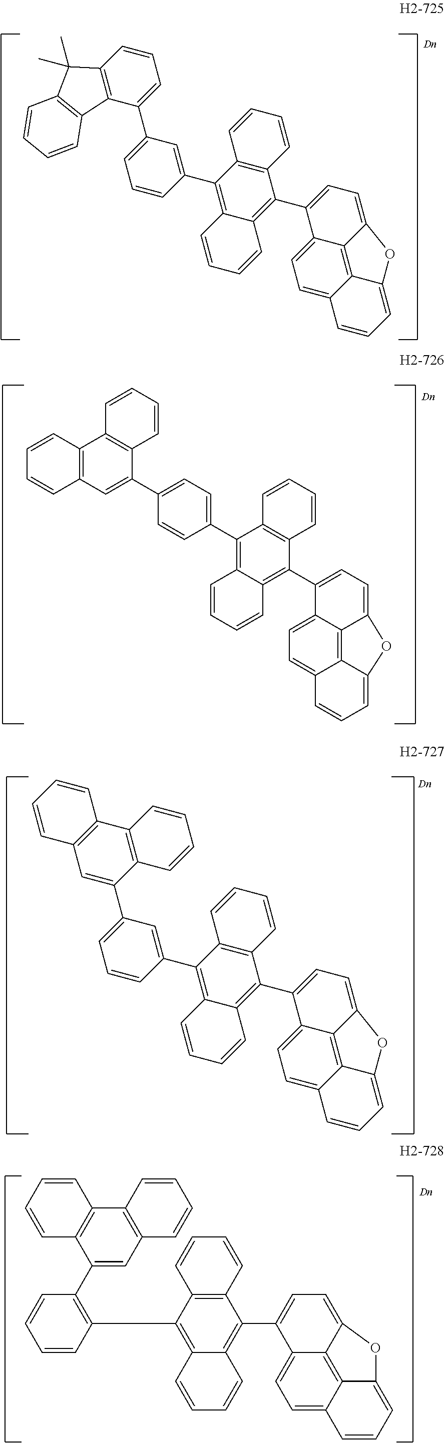 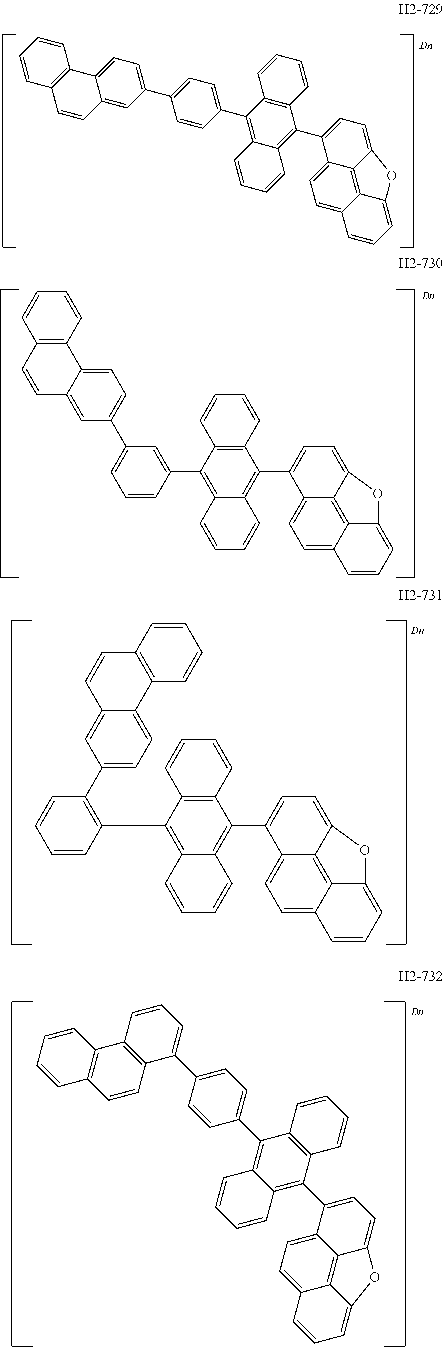 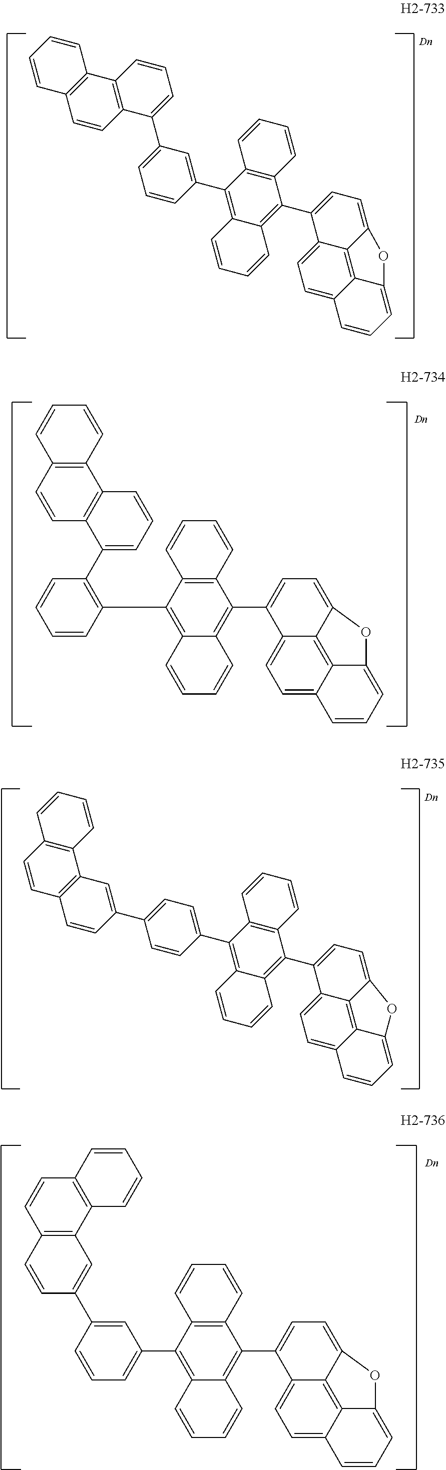 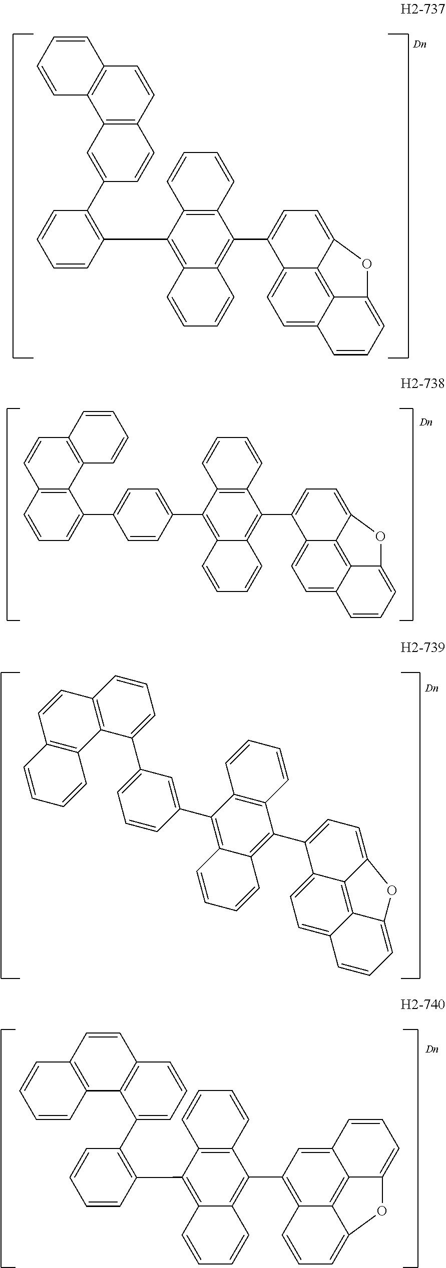 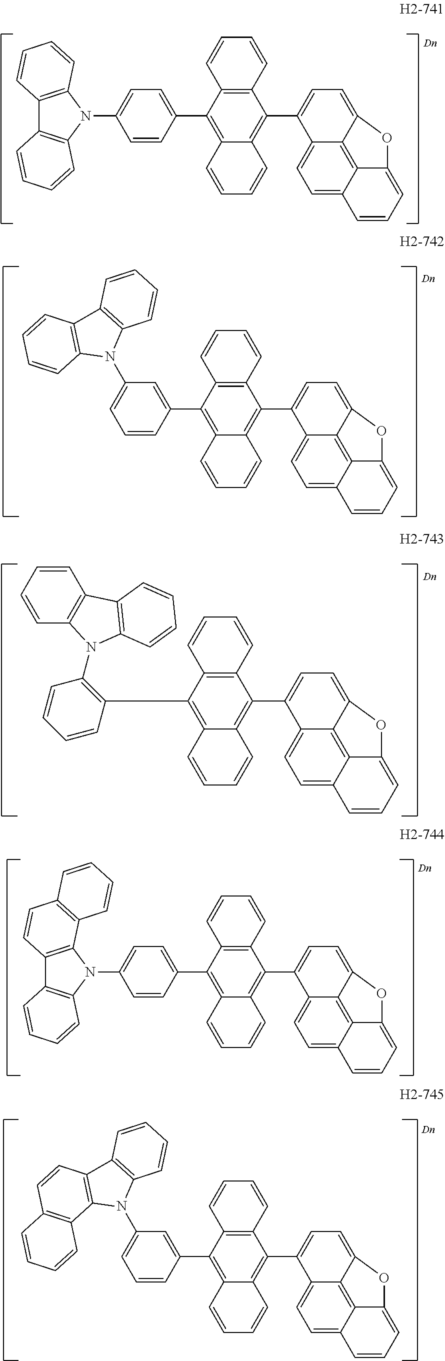 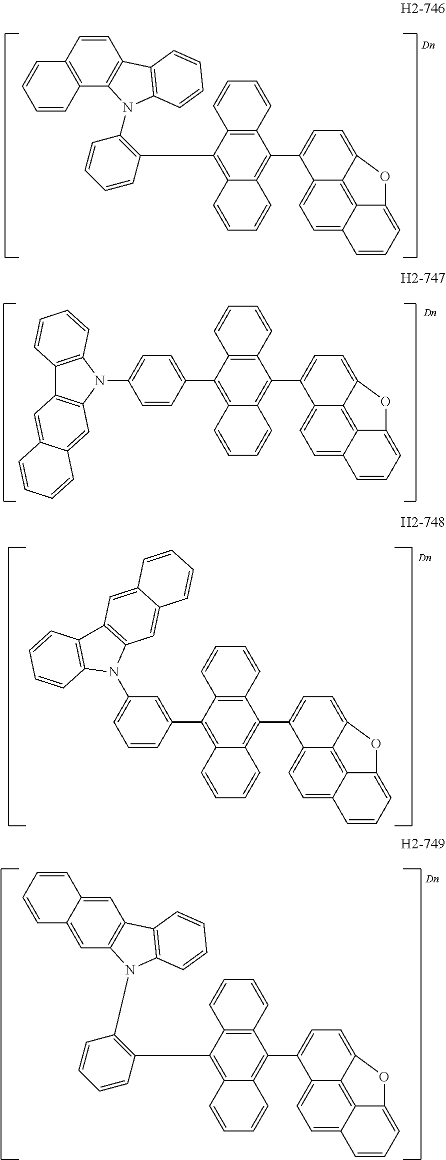 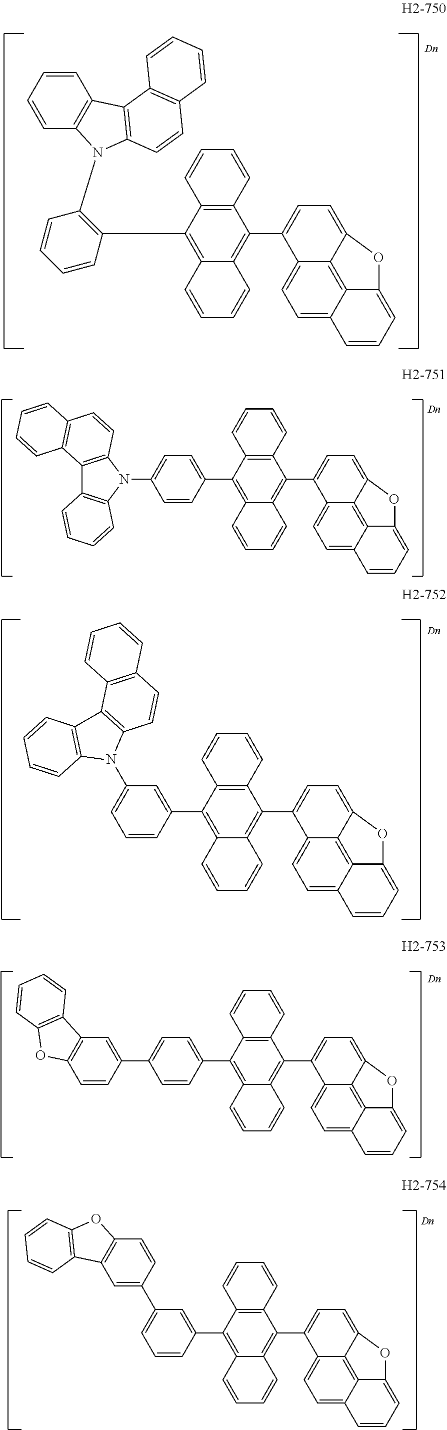 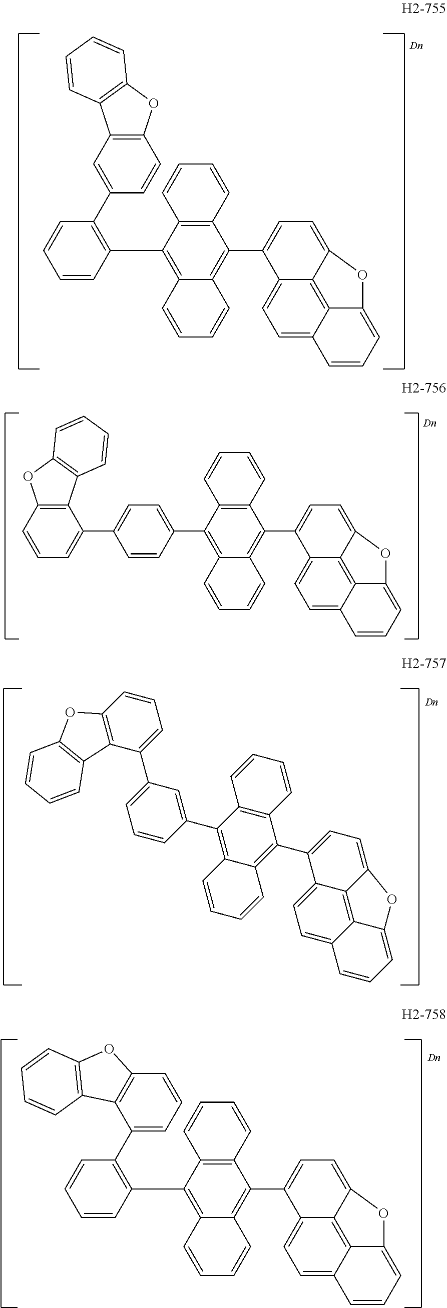 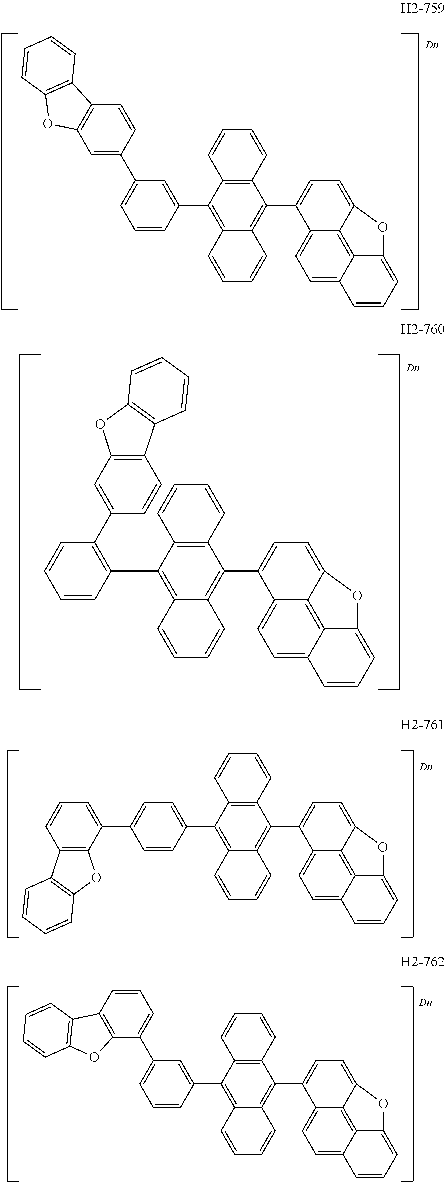 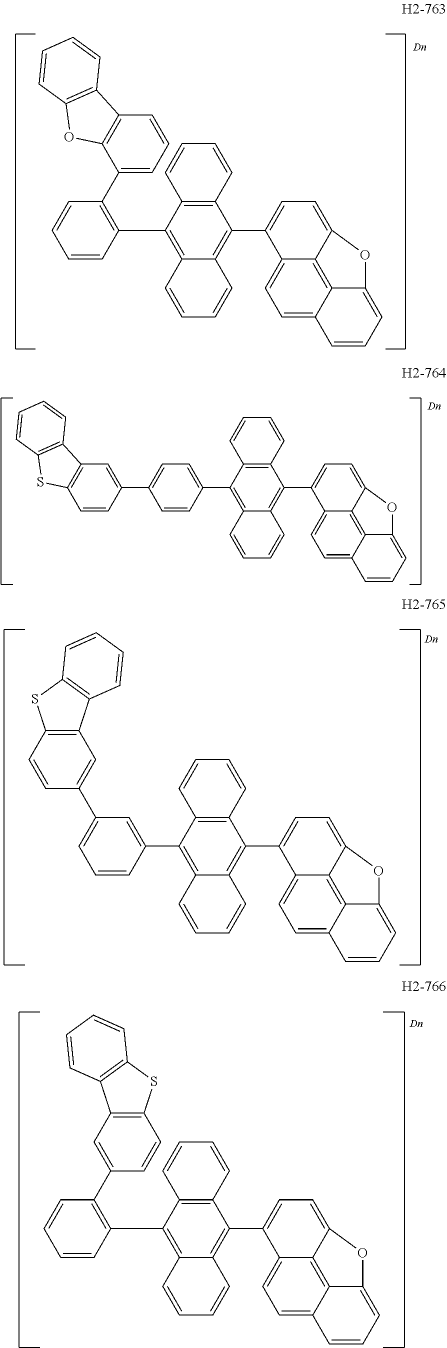 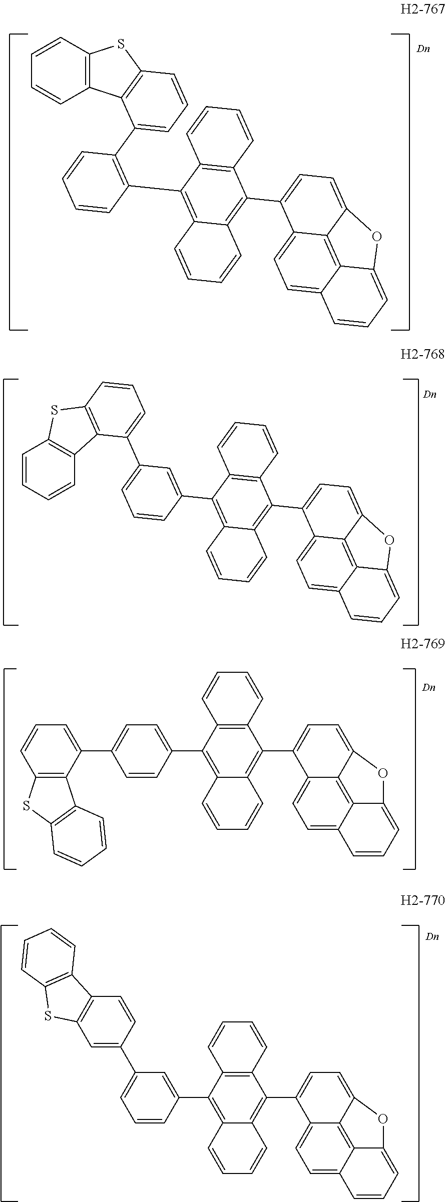 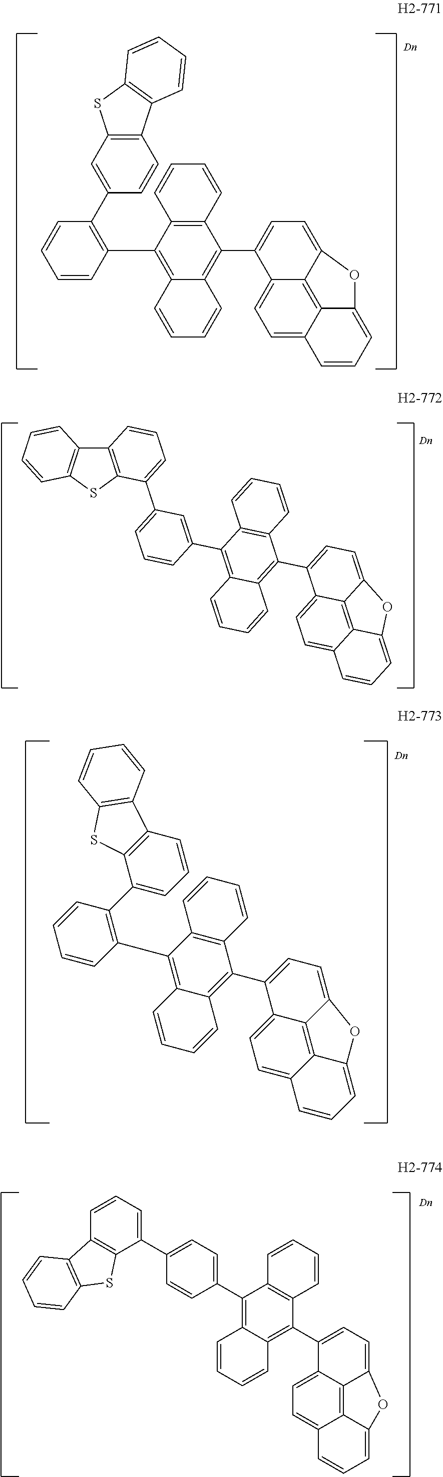 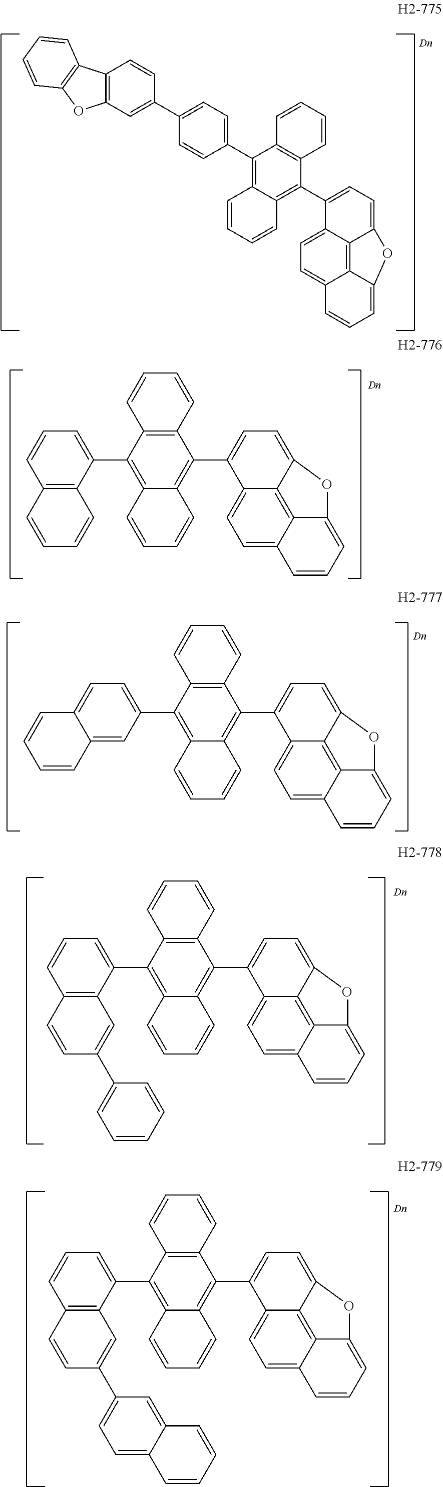 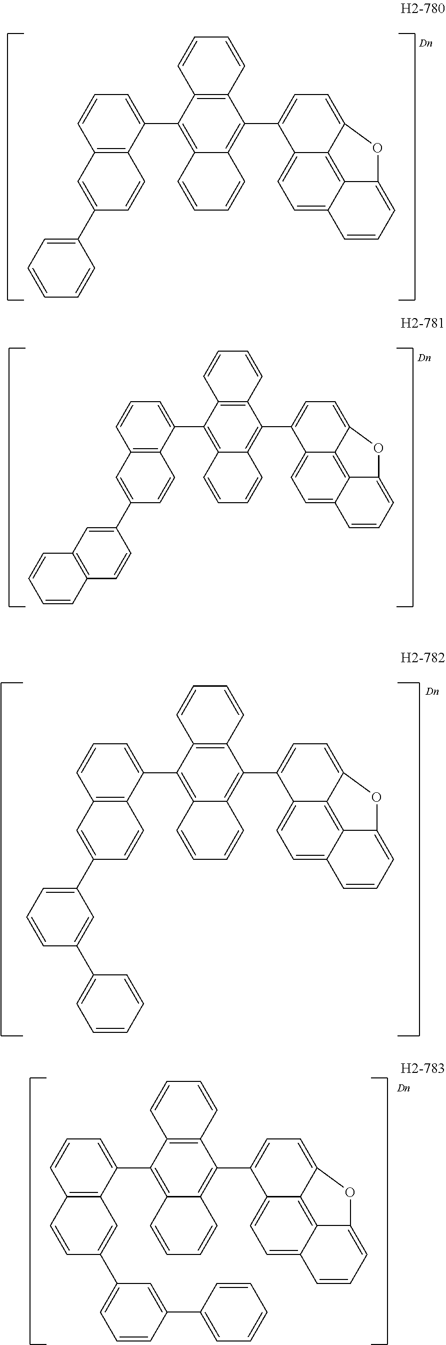 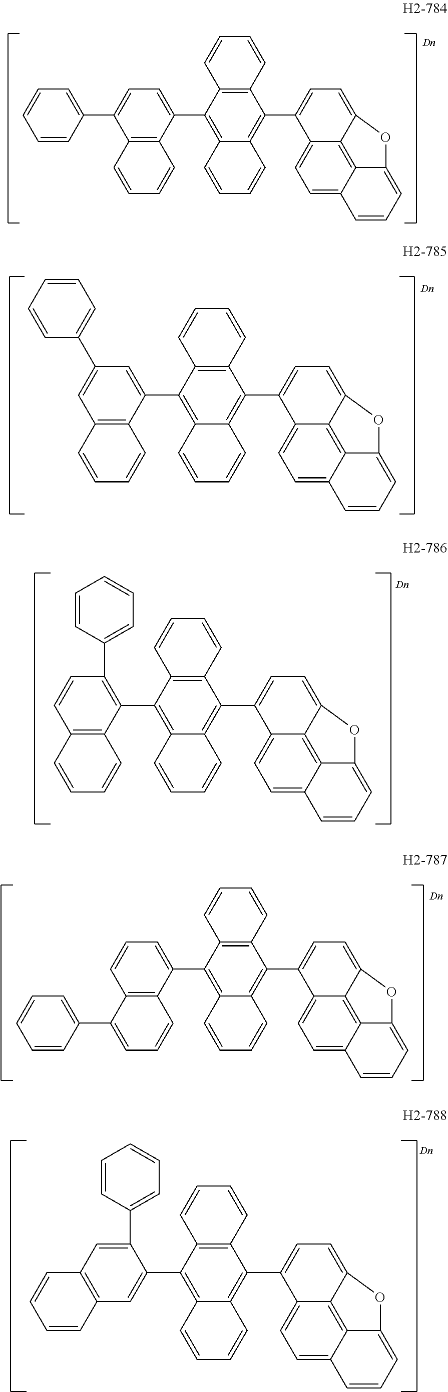 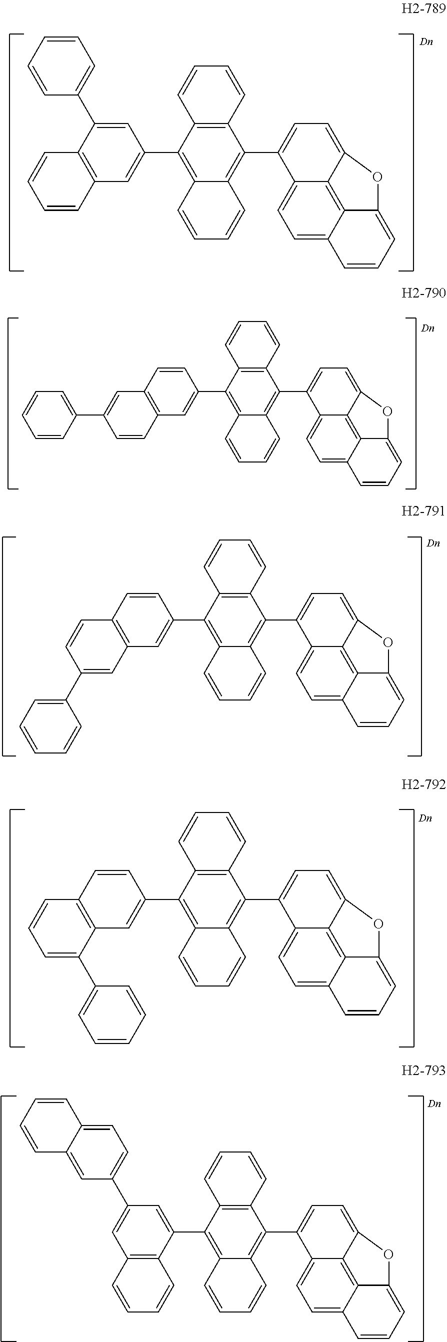 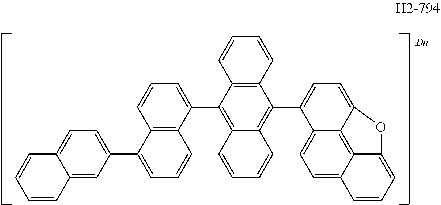 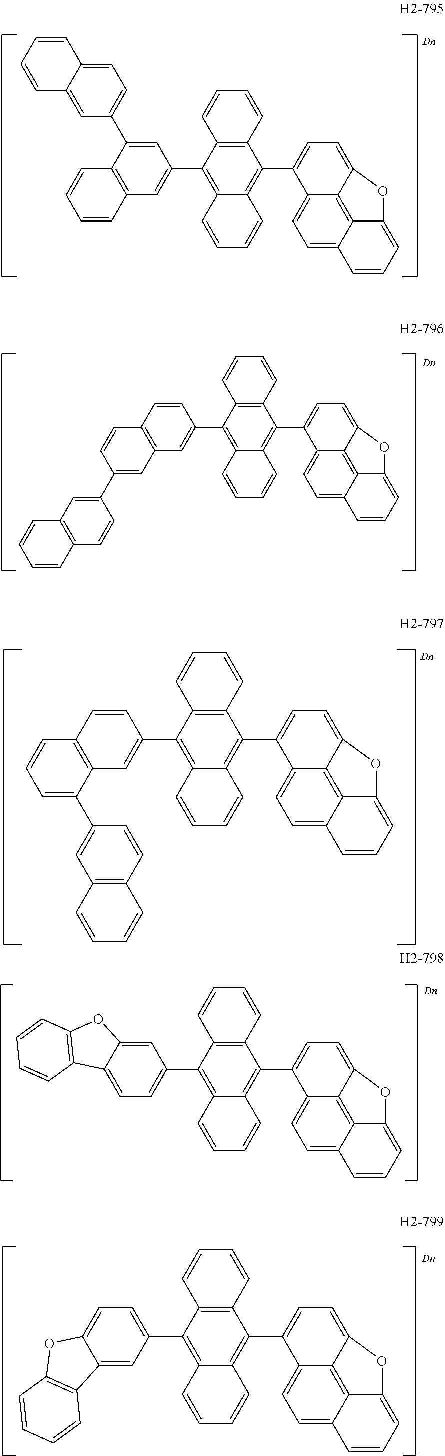 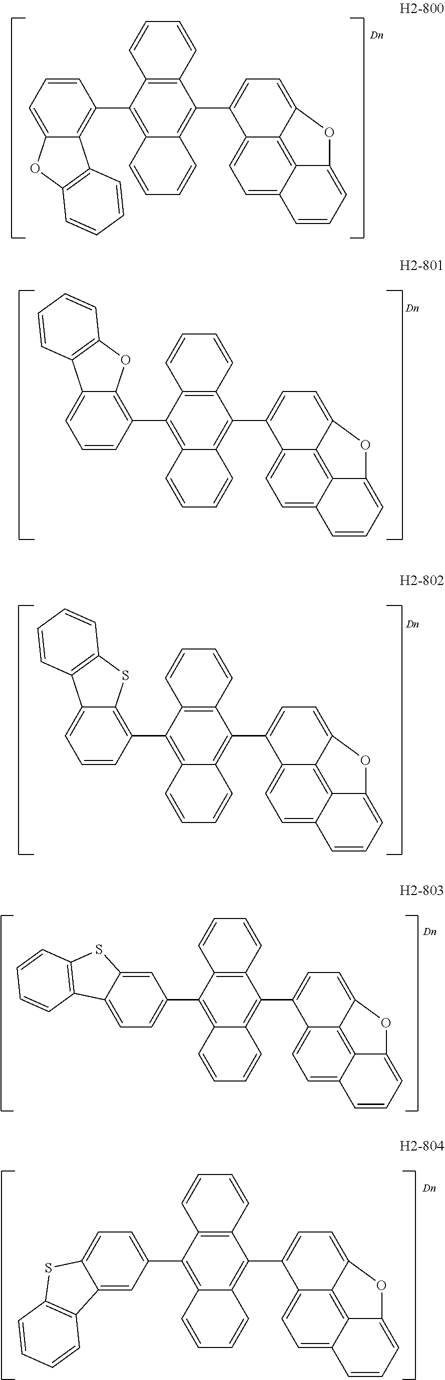 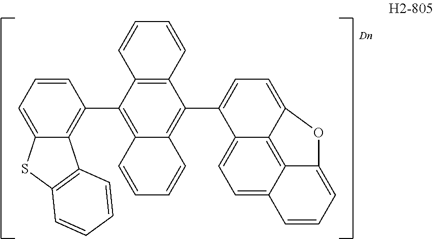 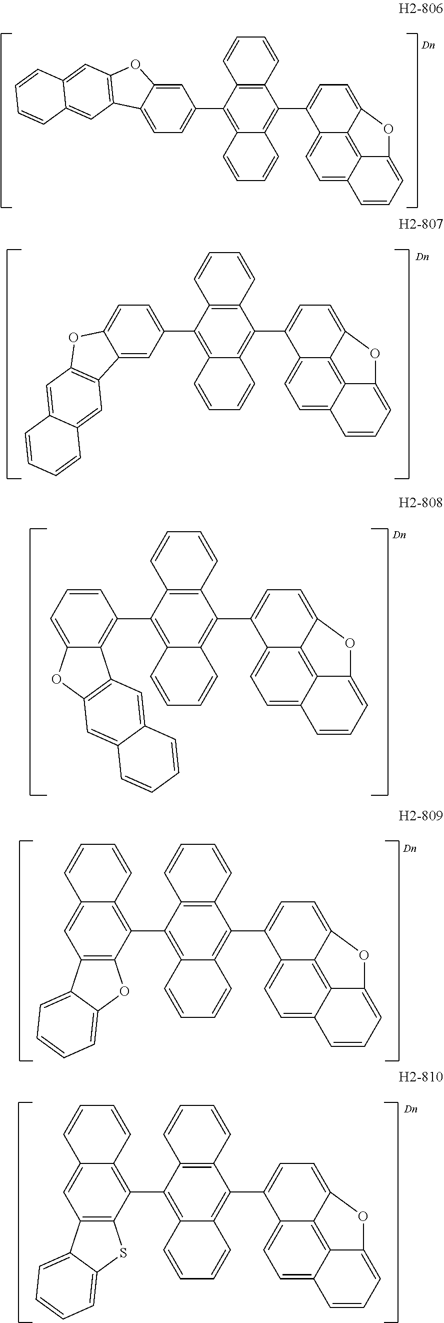 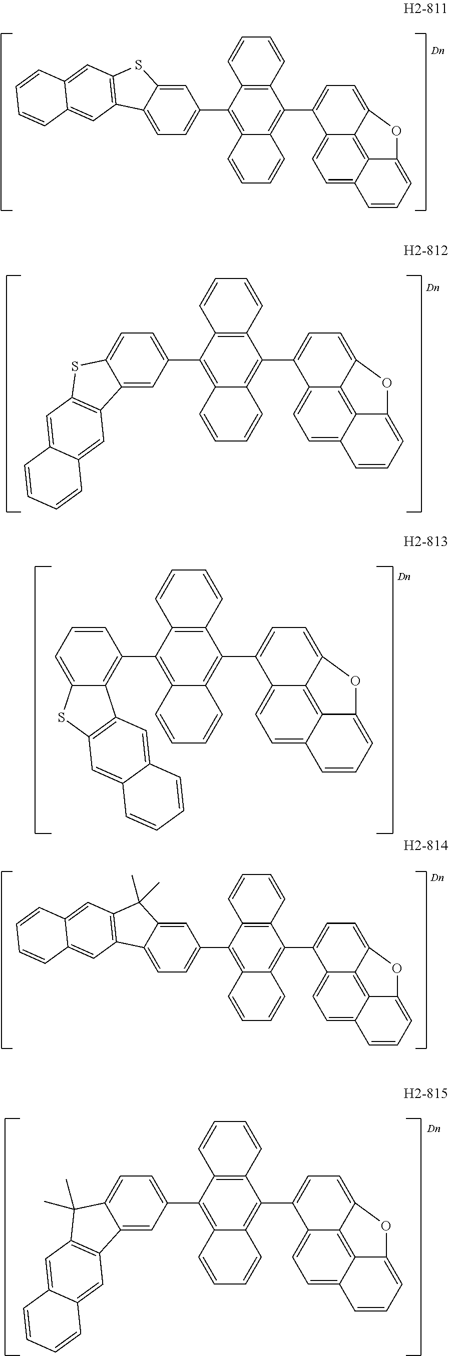 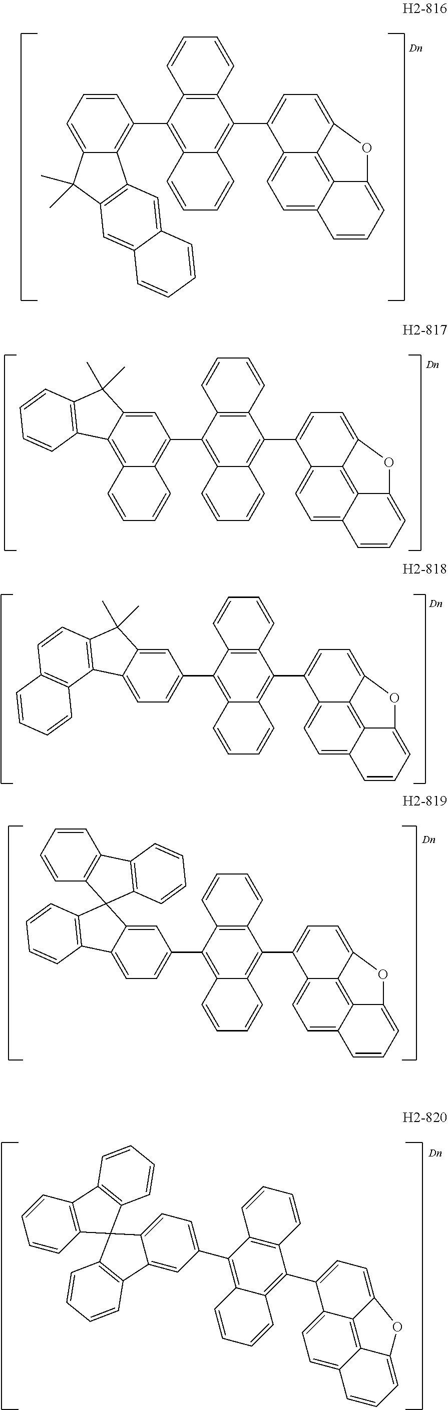 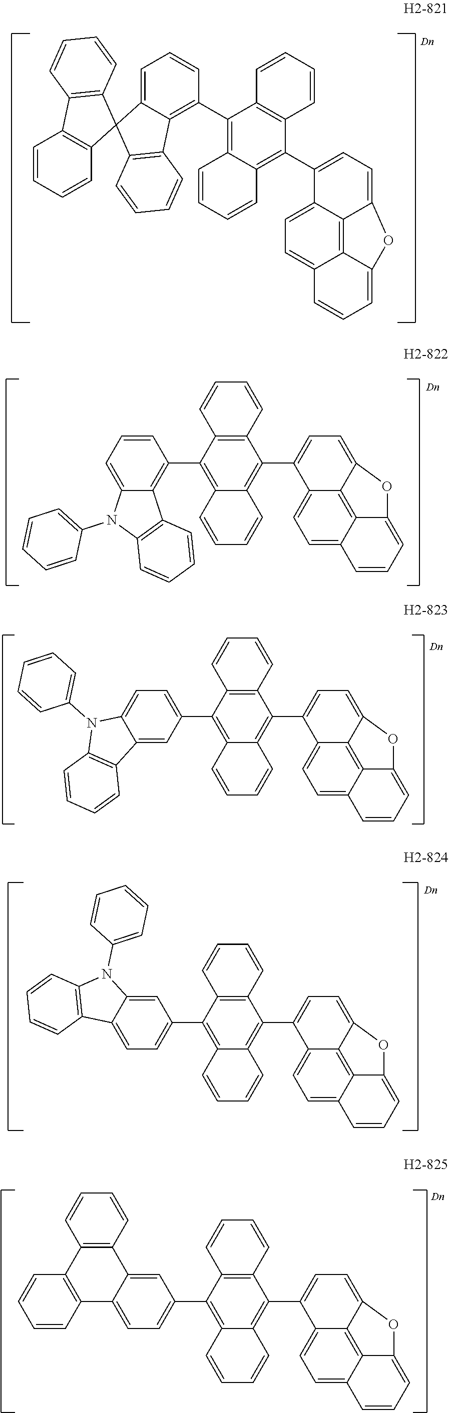 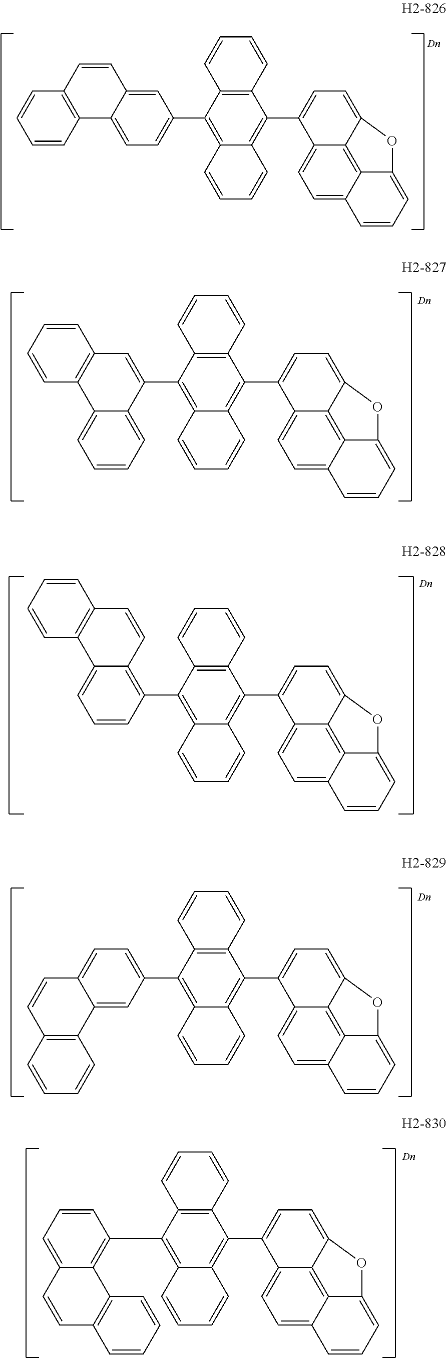 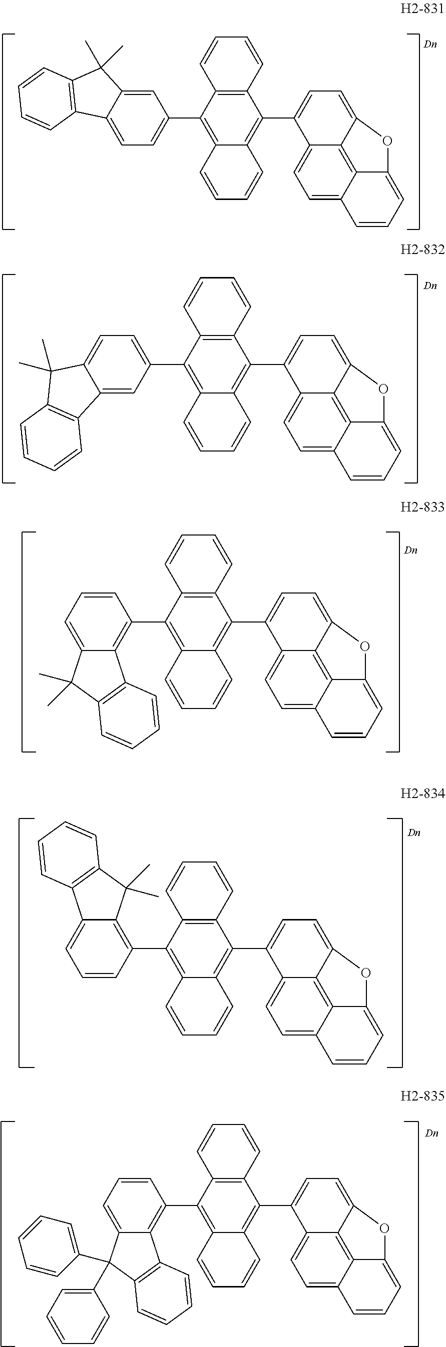 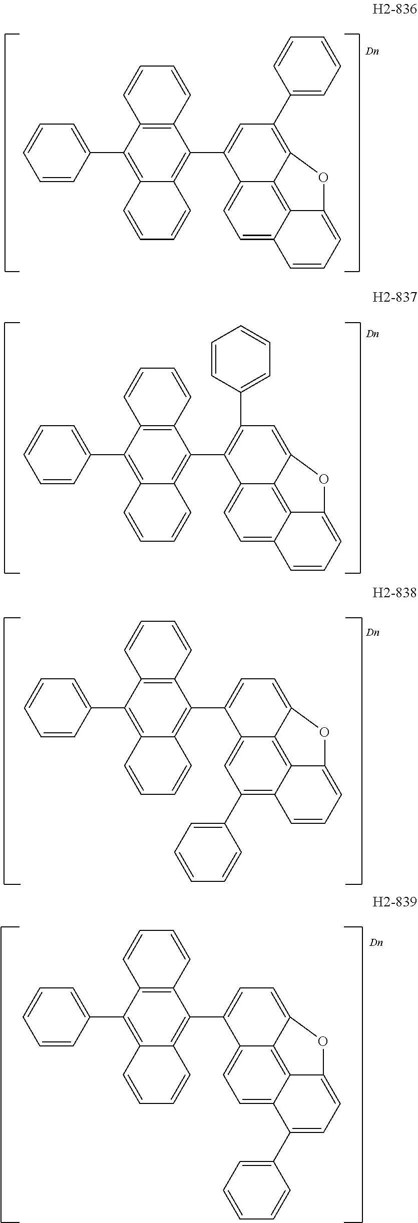 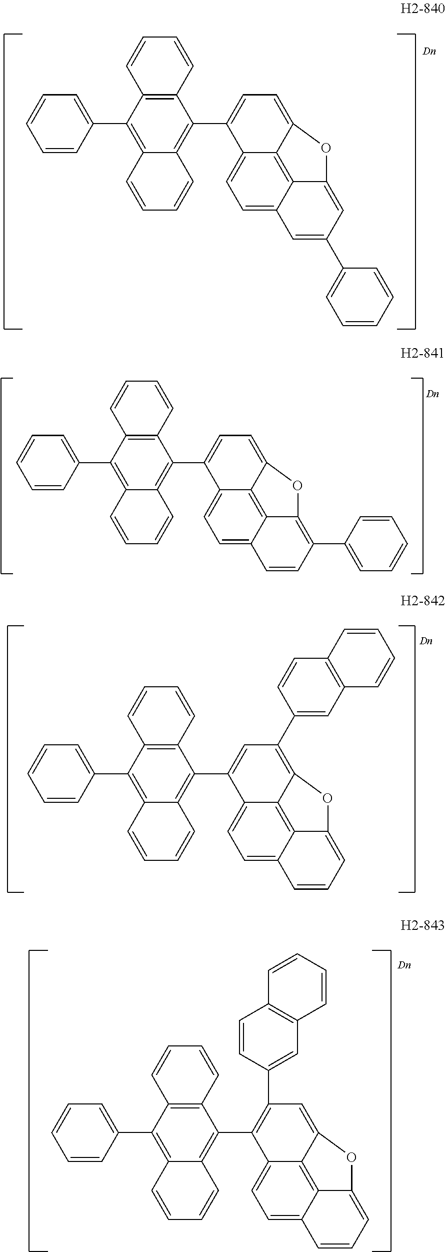 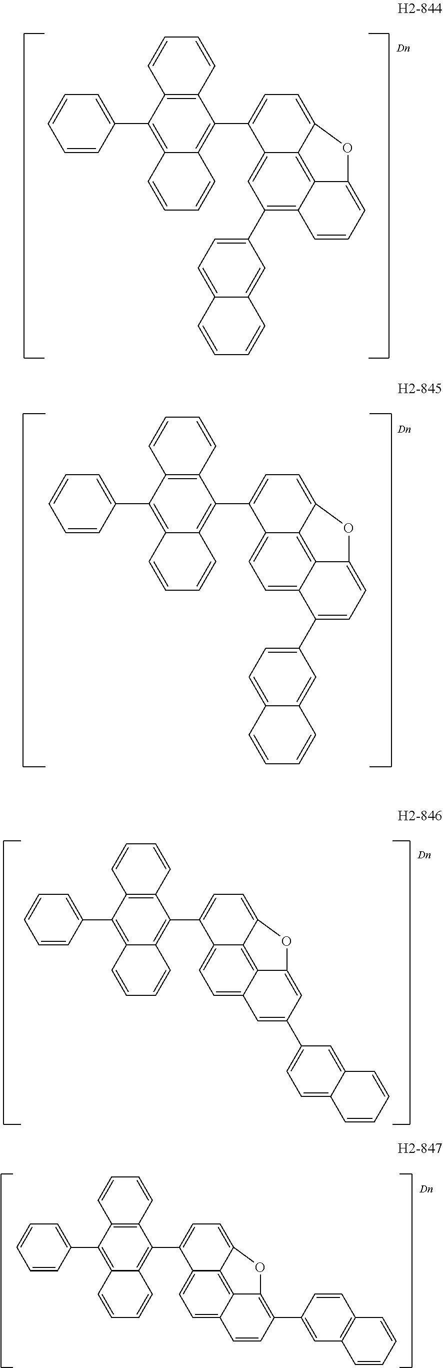 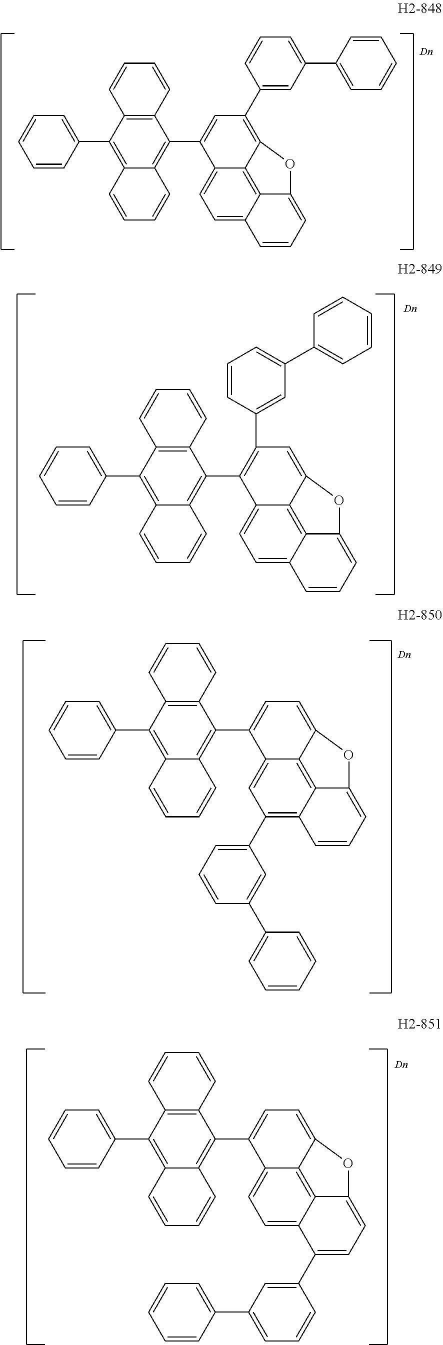 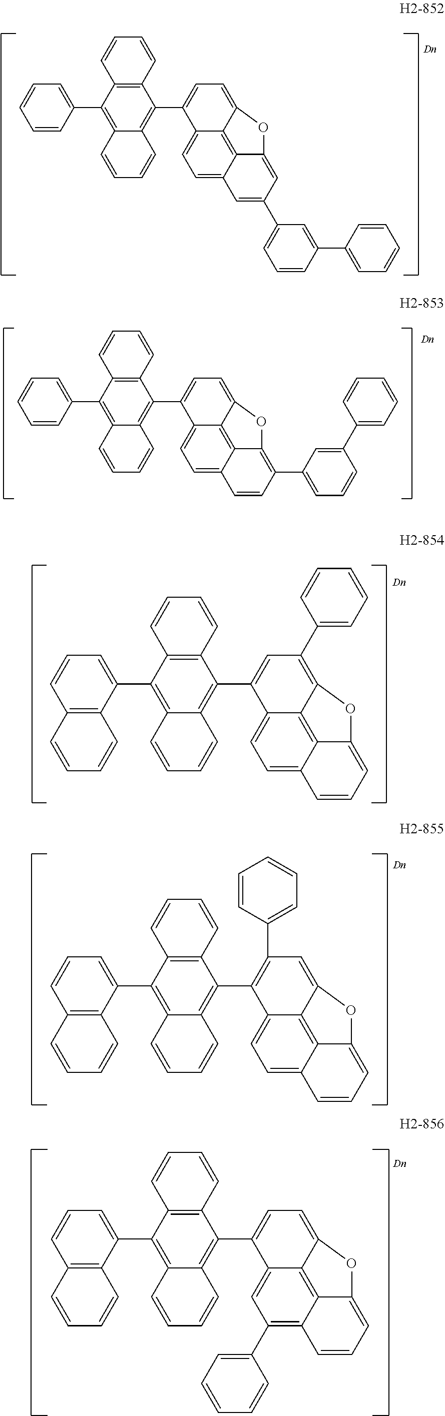 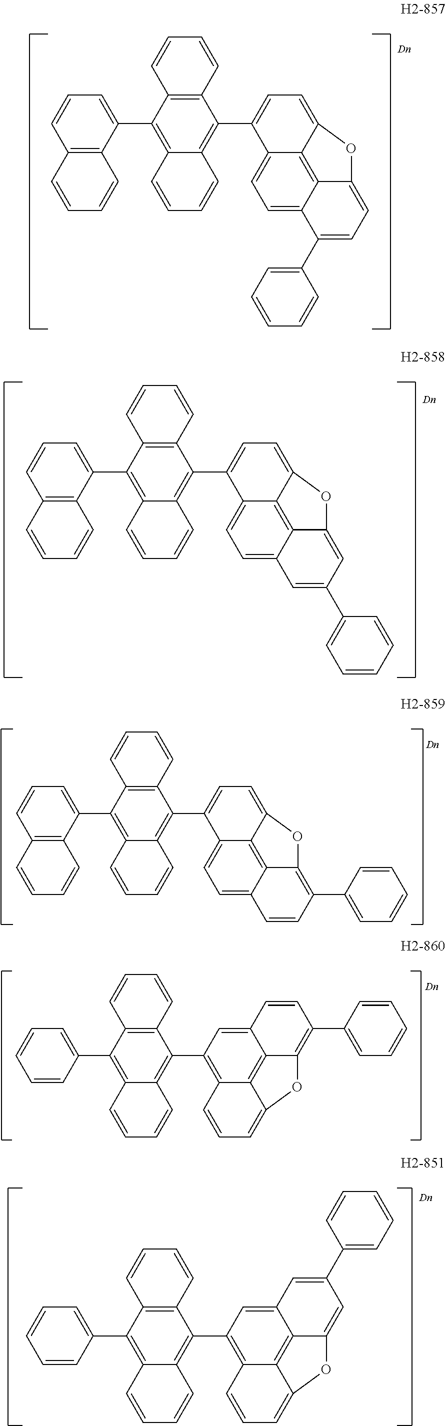 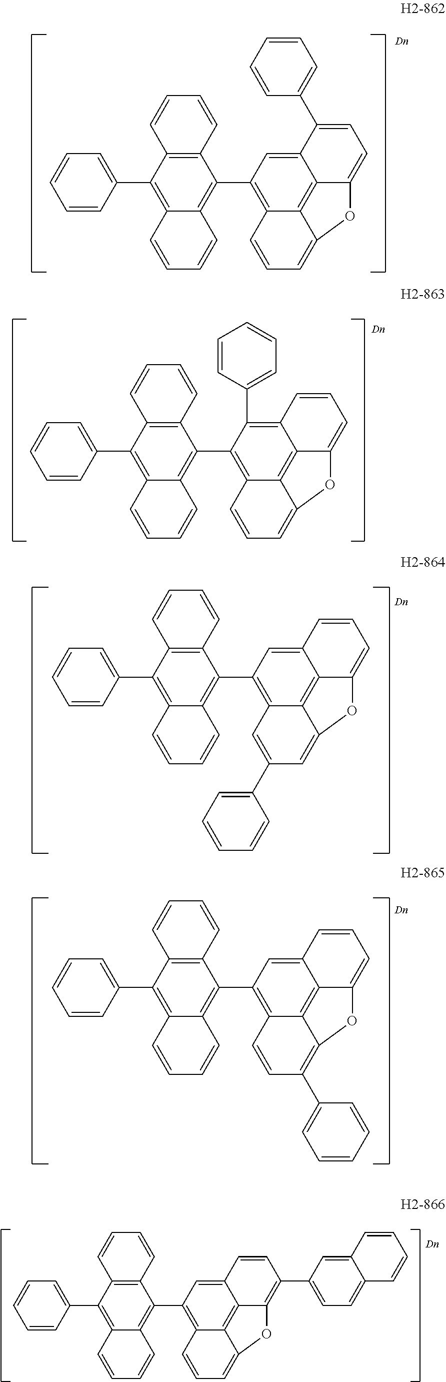 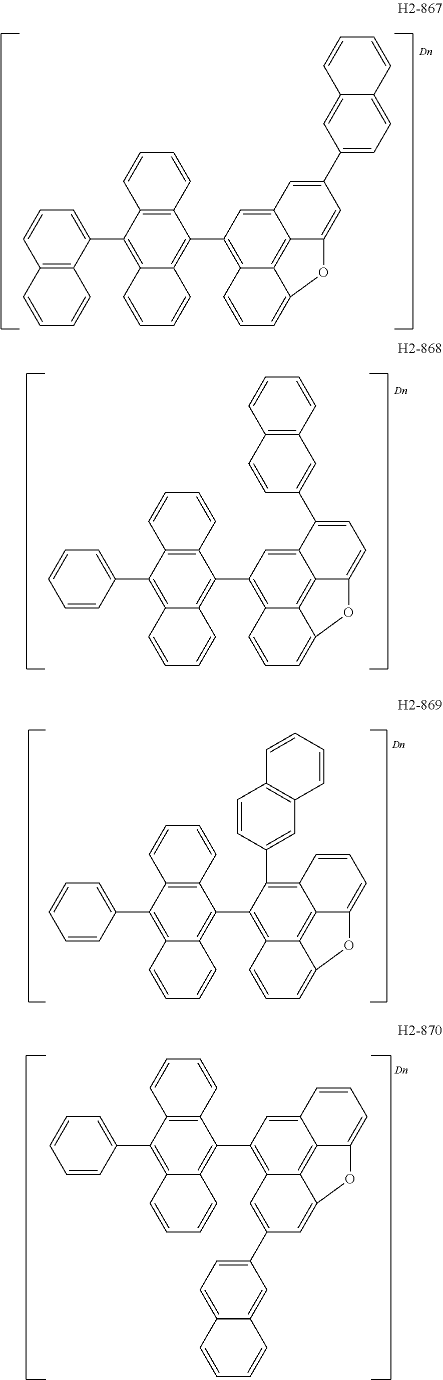 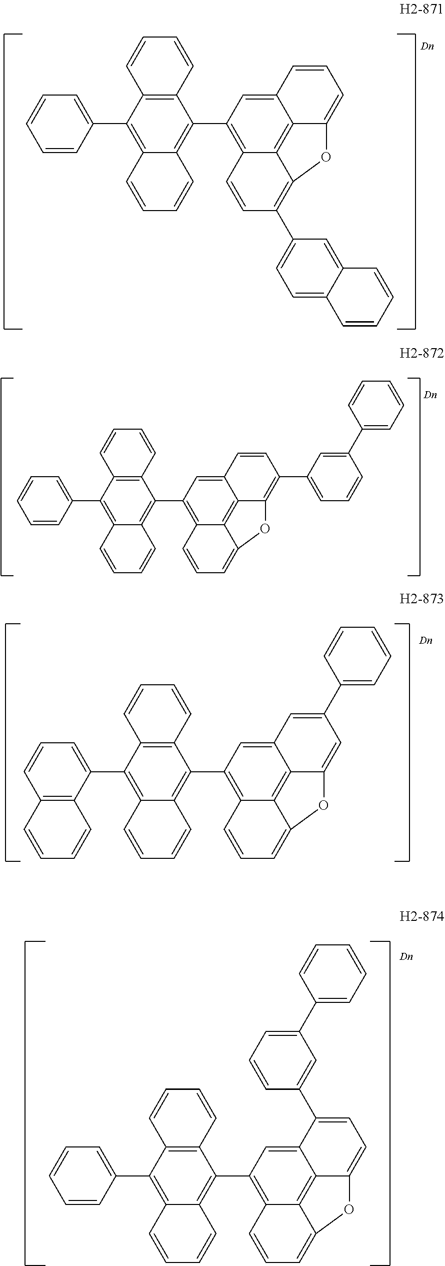 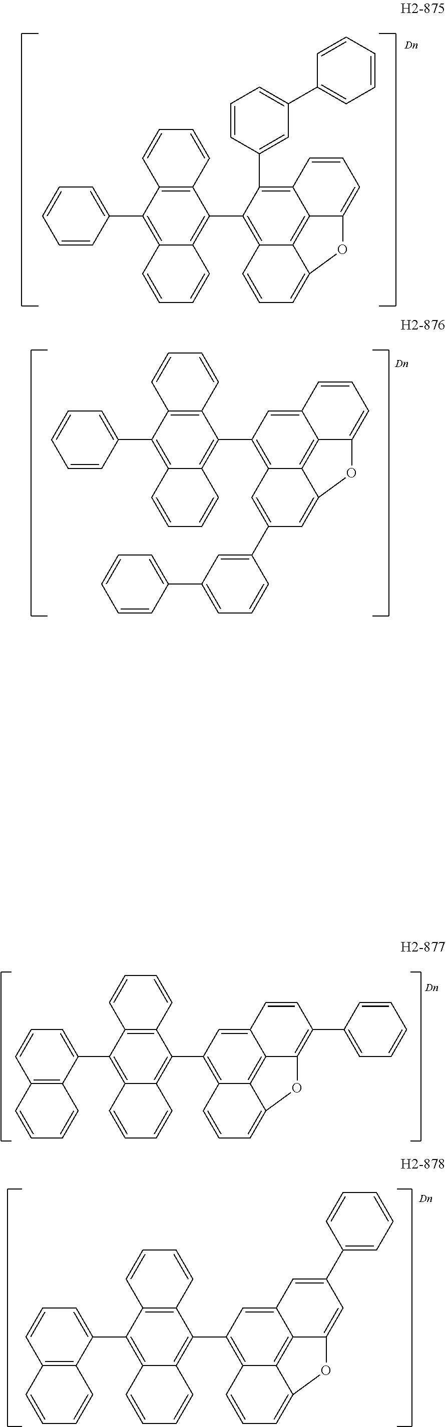 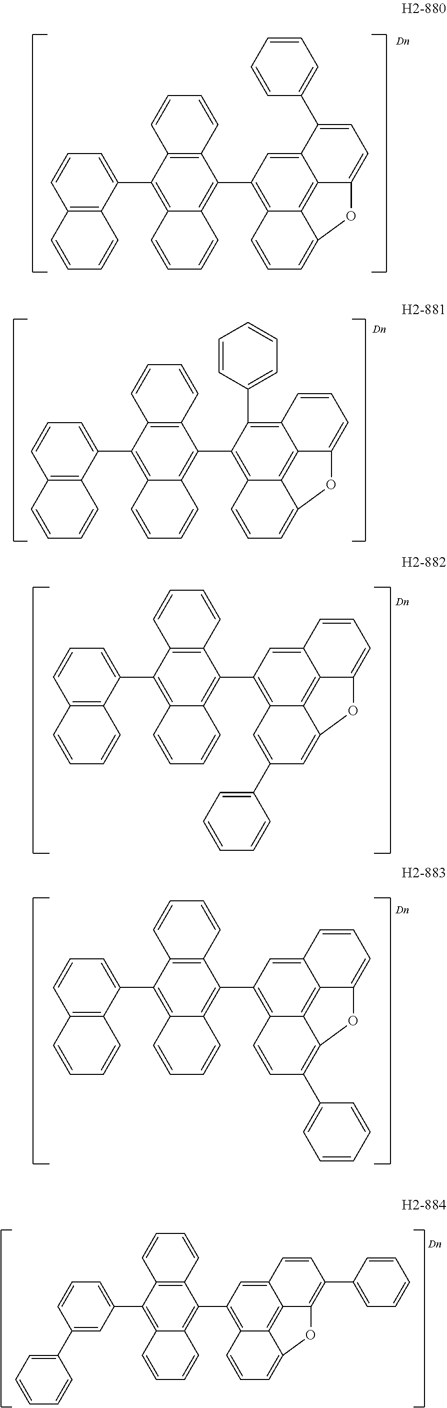 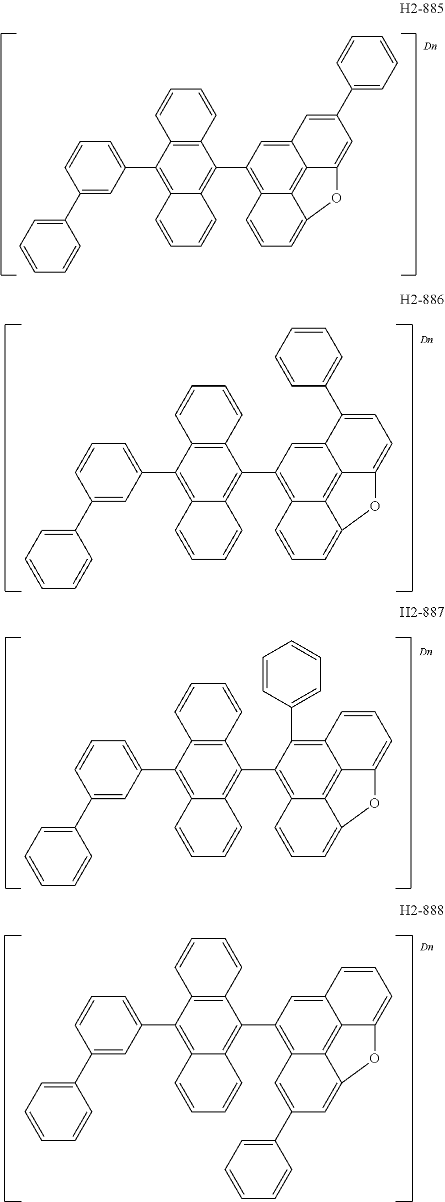 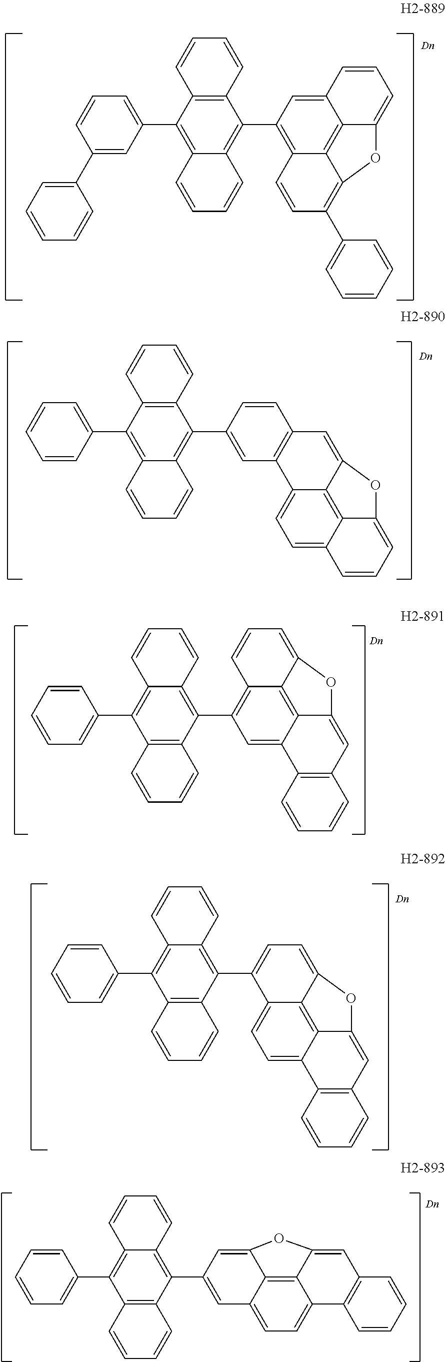 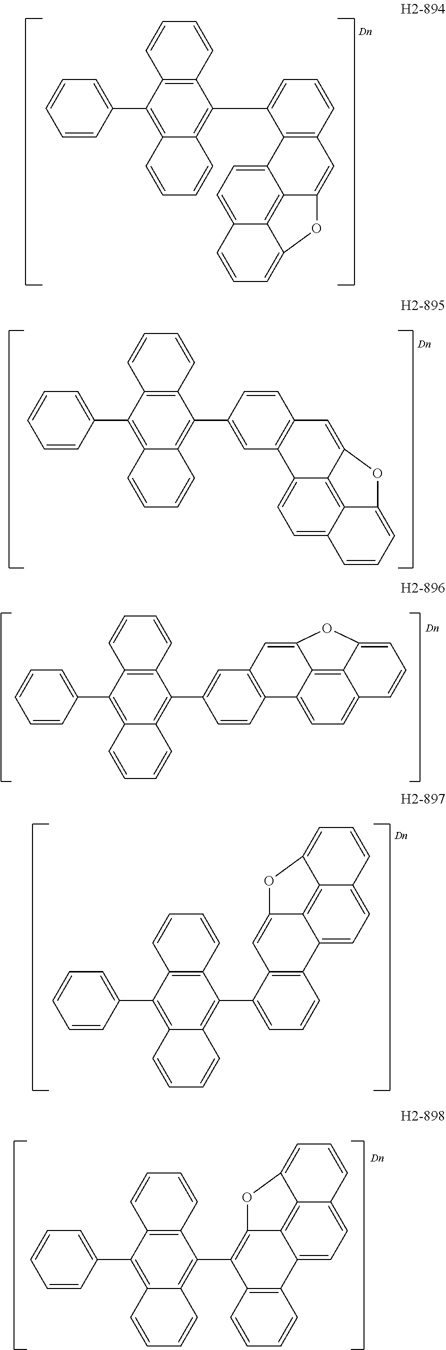 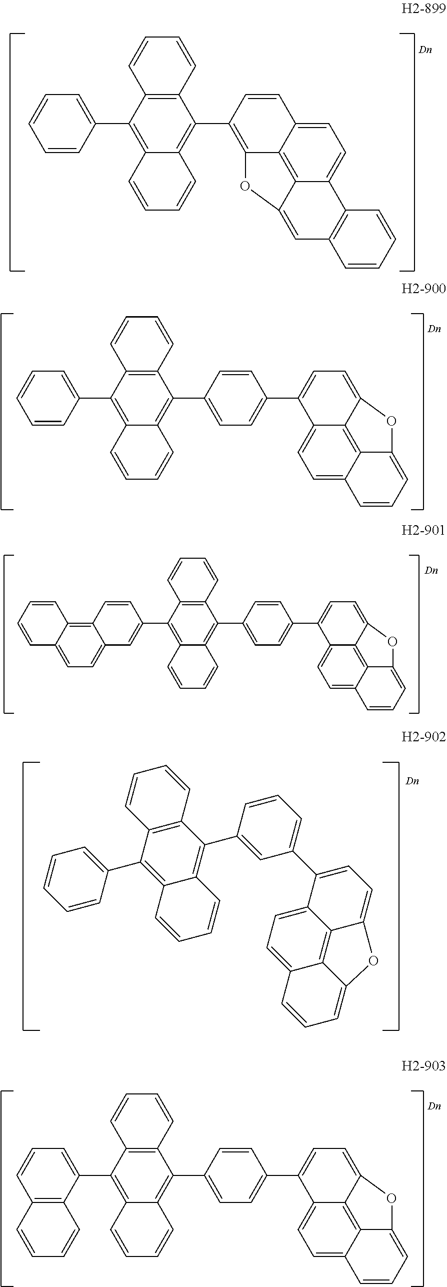 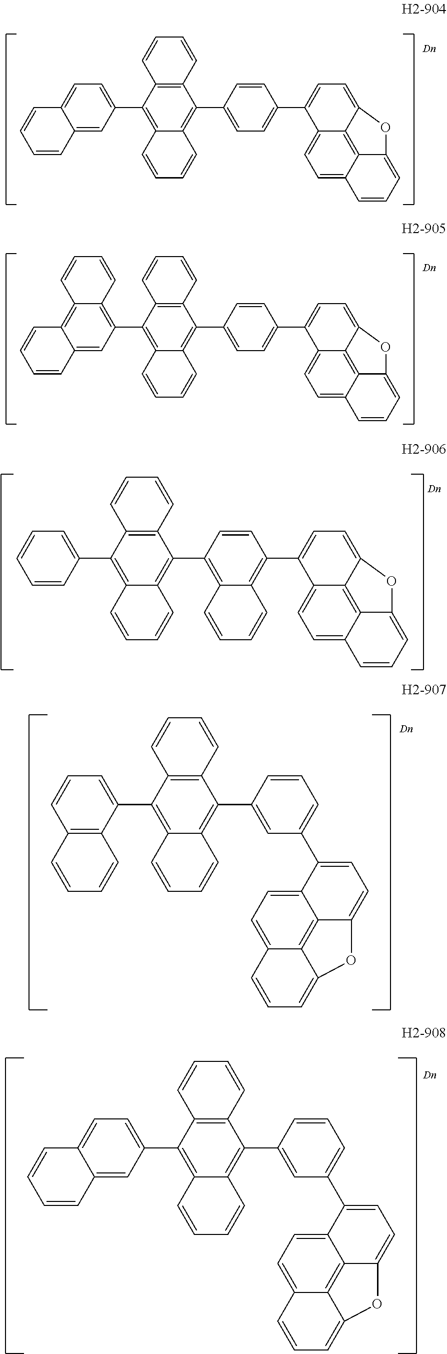 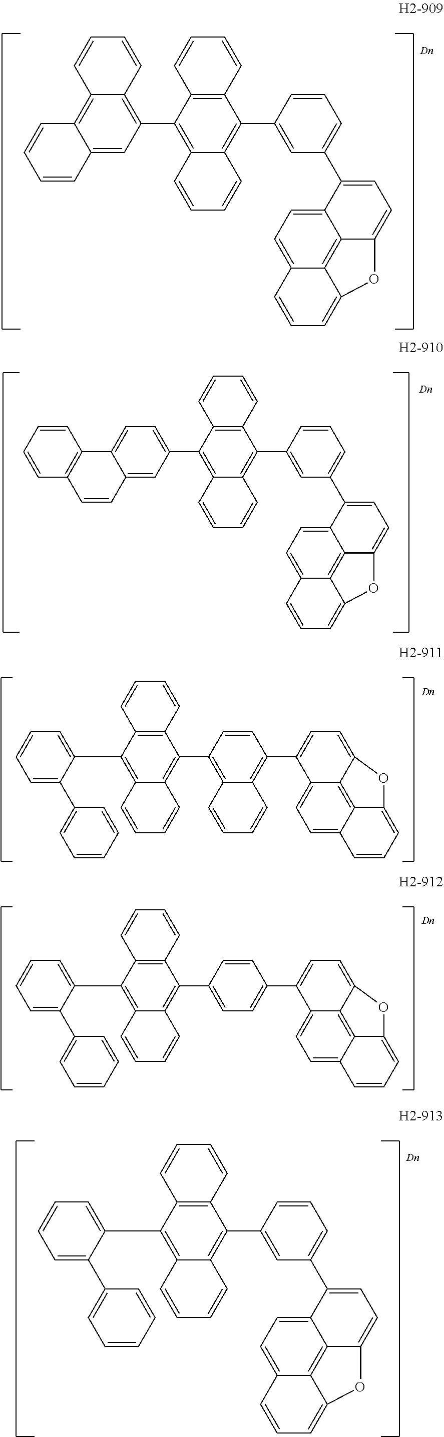 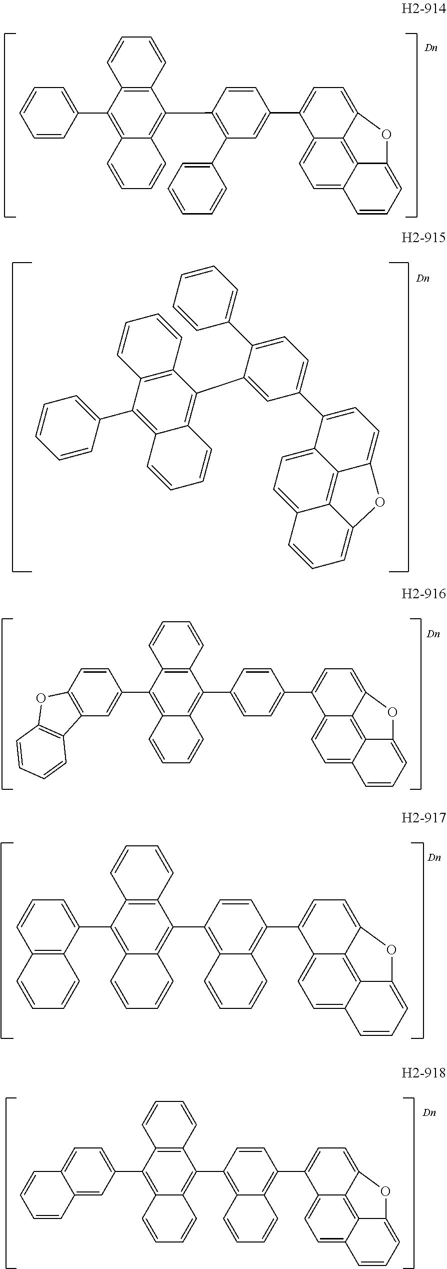 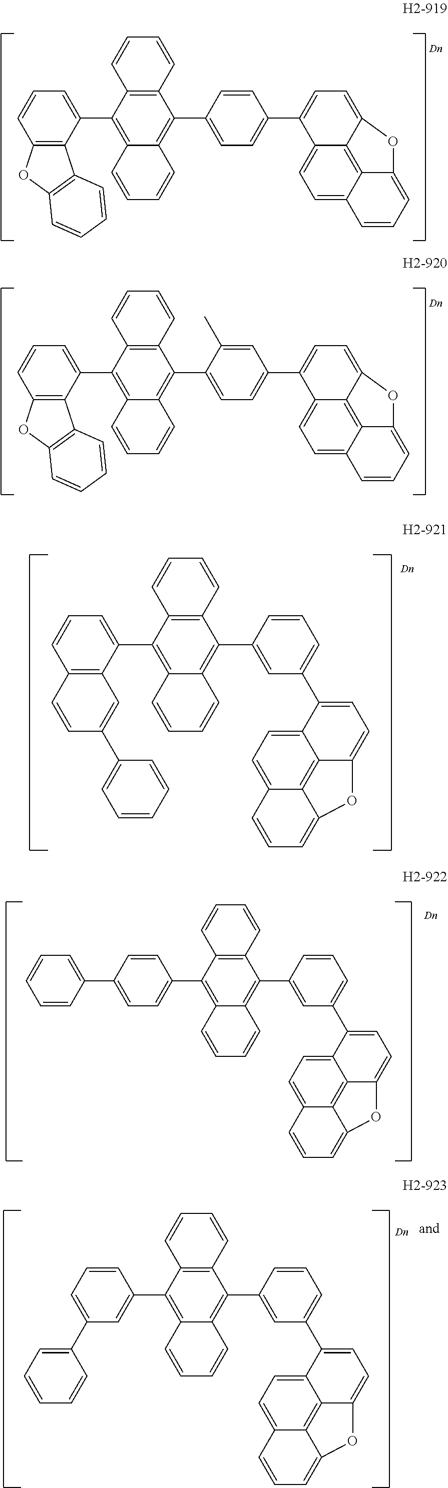 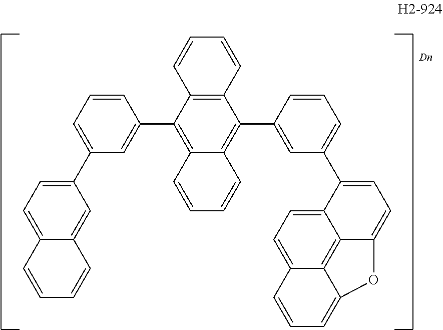
wherein Dₙ signifies that n hydrogens are replaced with deuterium, and n is an integer from 1 to the maximum number of hydrogens in the compound.

11. The plurality of host materials according to claim 1, wherein the compound represented by Formula 2 is at least one selected from the following compounds: 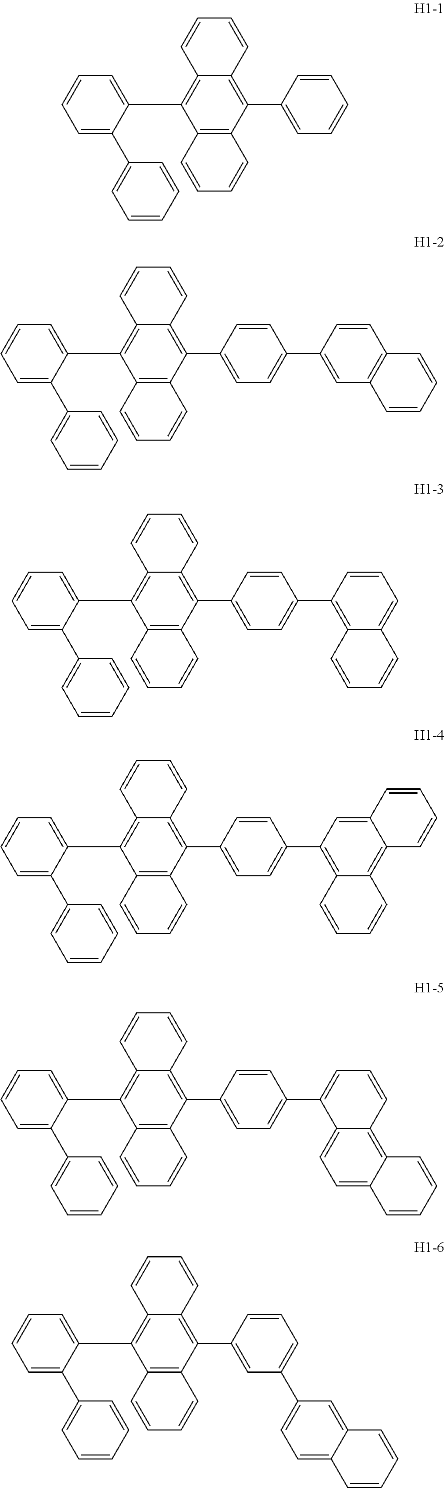 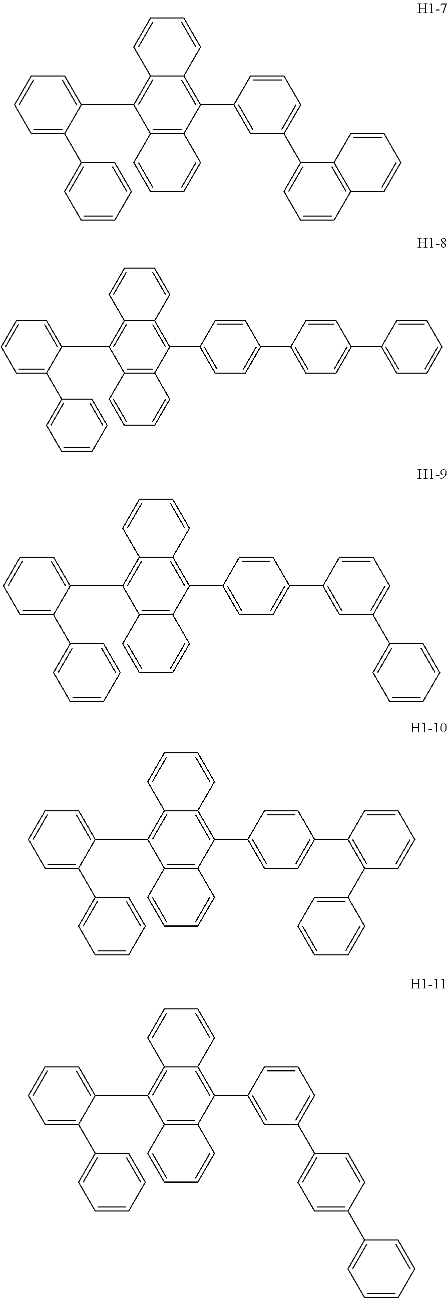 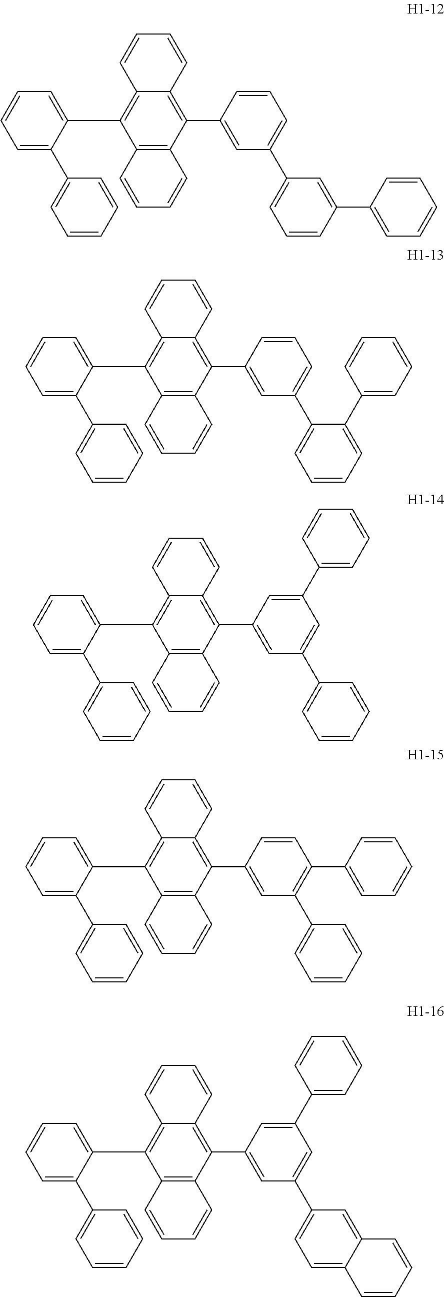 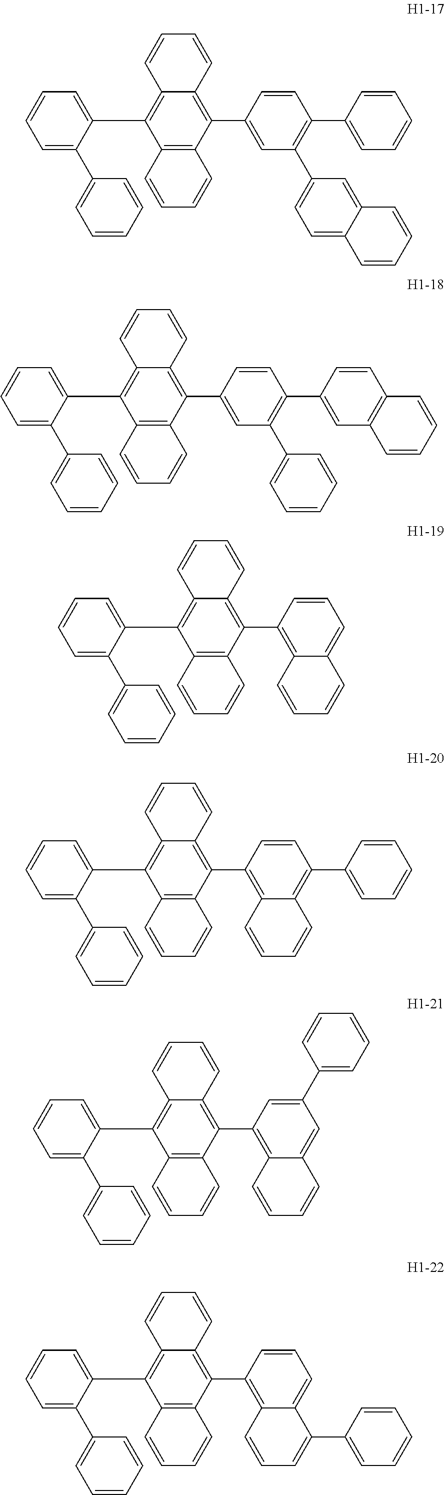 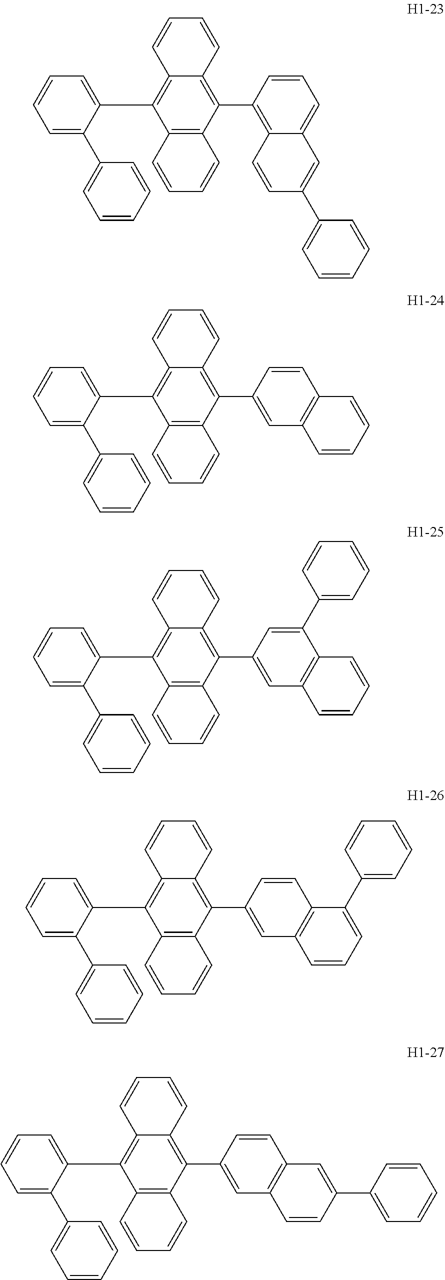 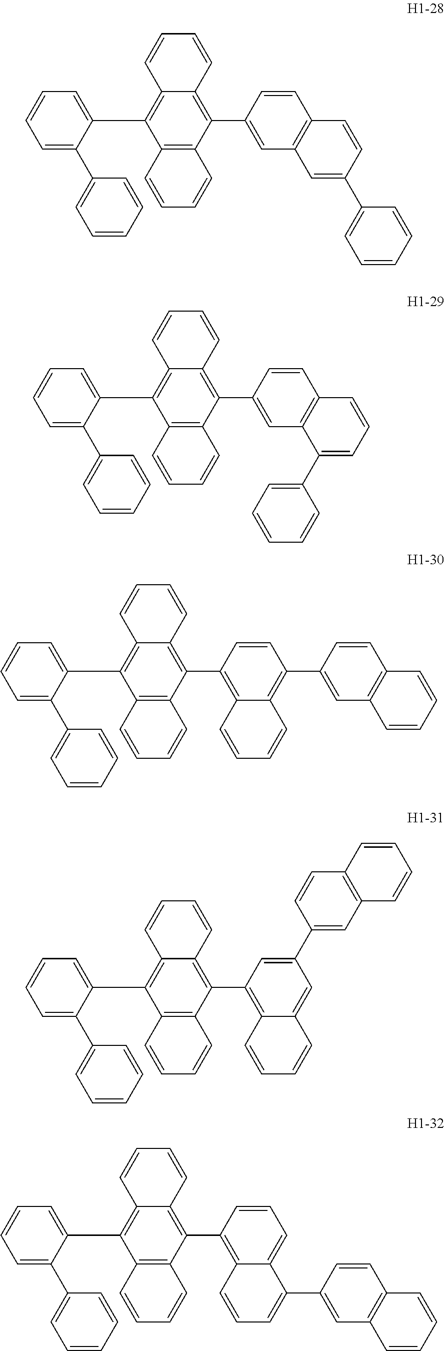 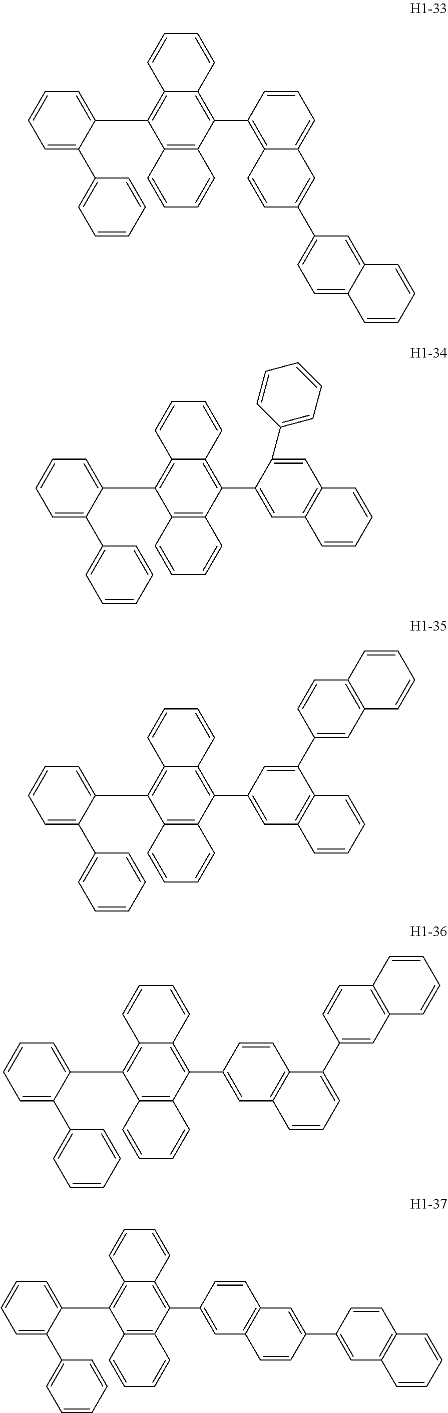 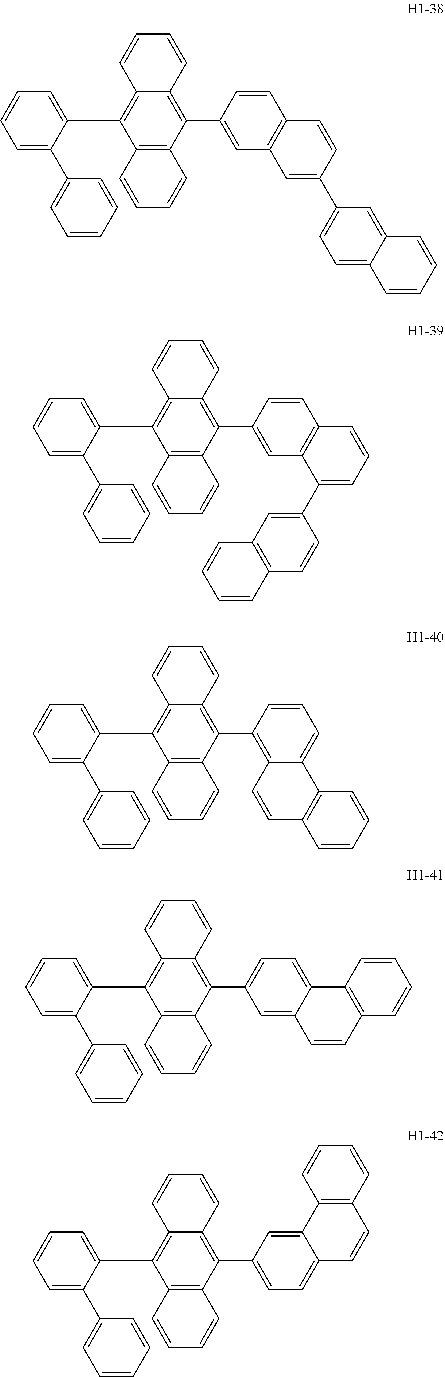 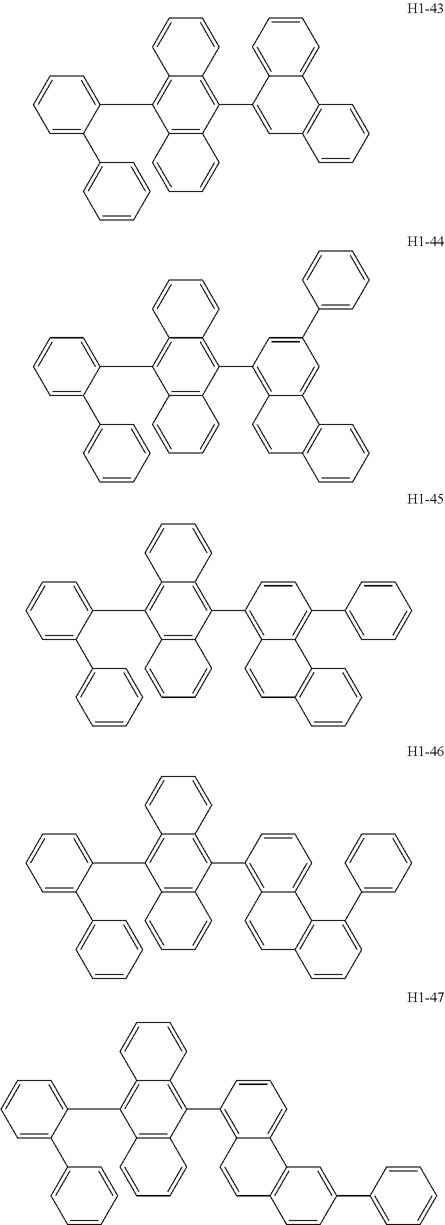 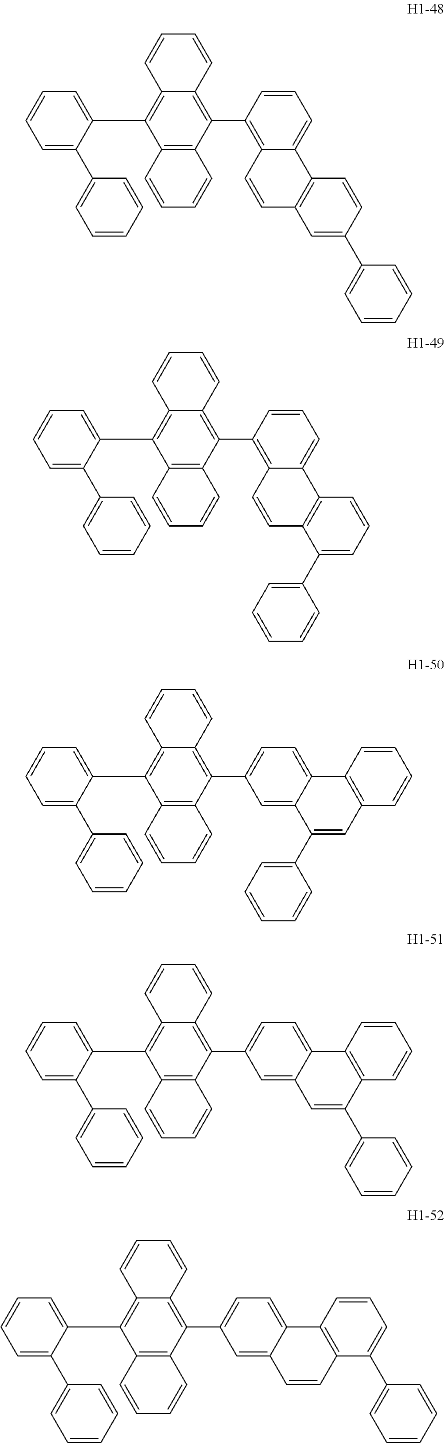 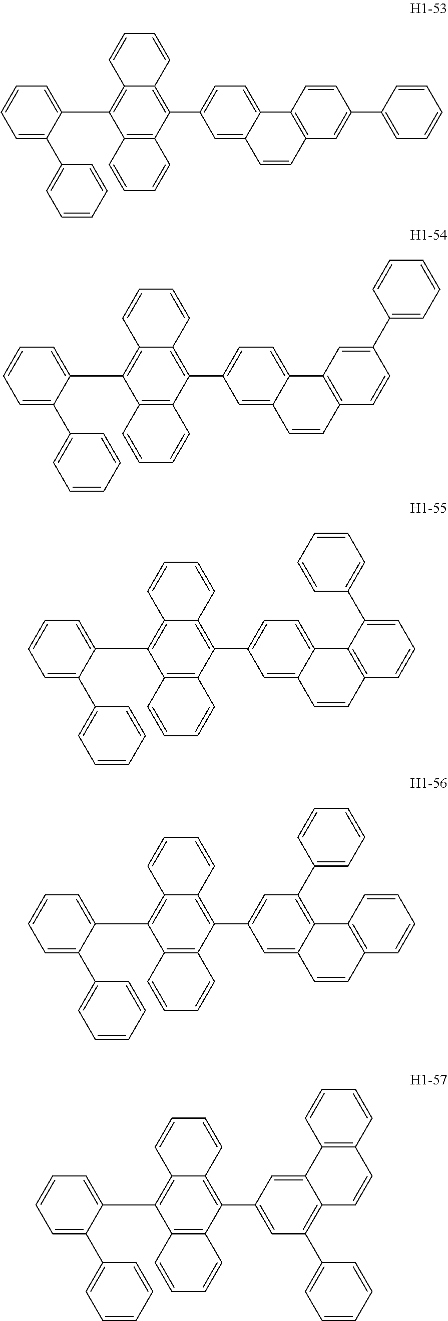 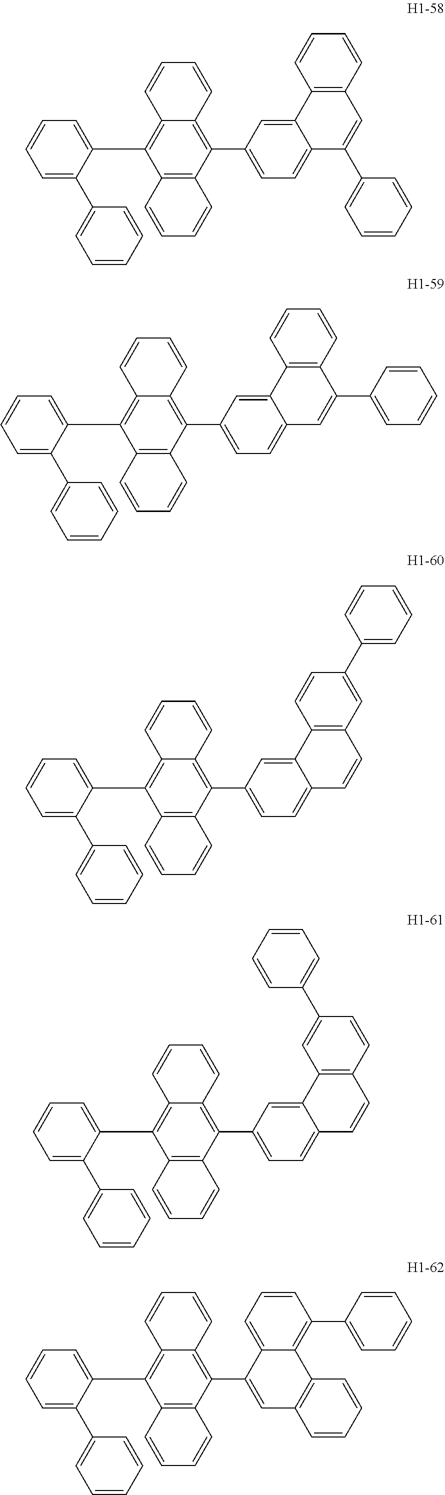 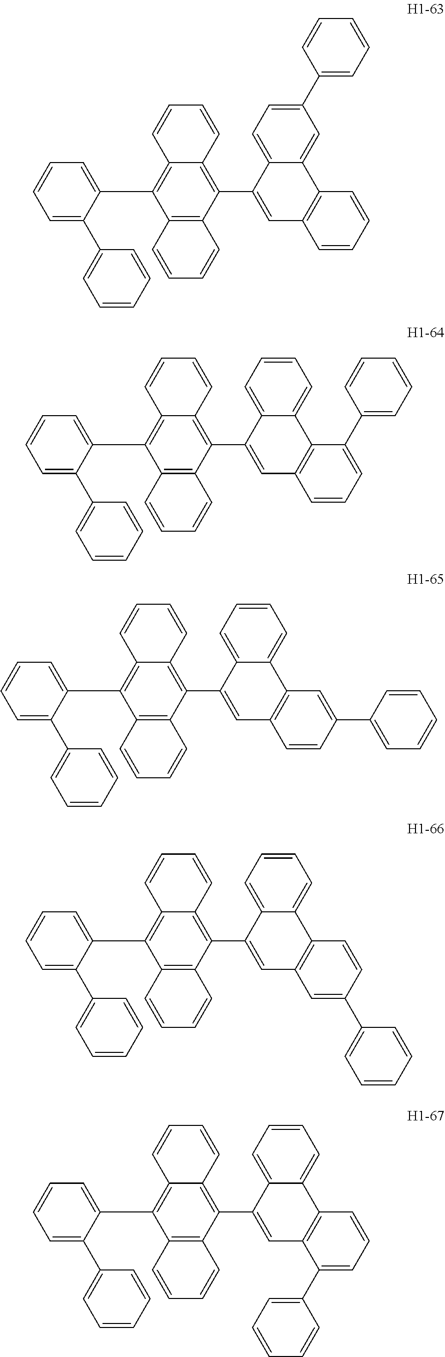 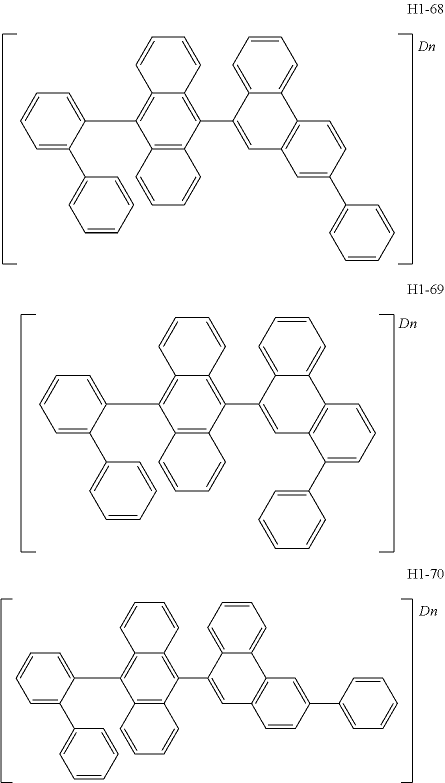 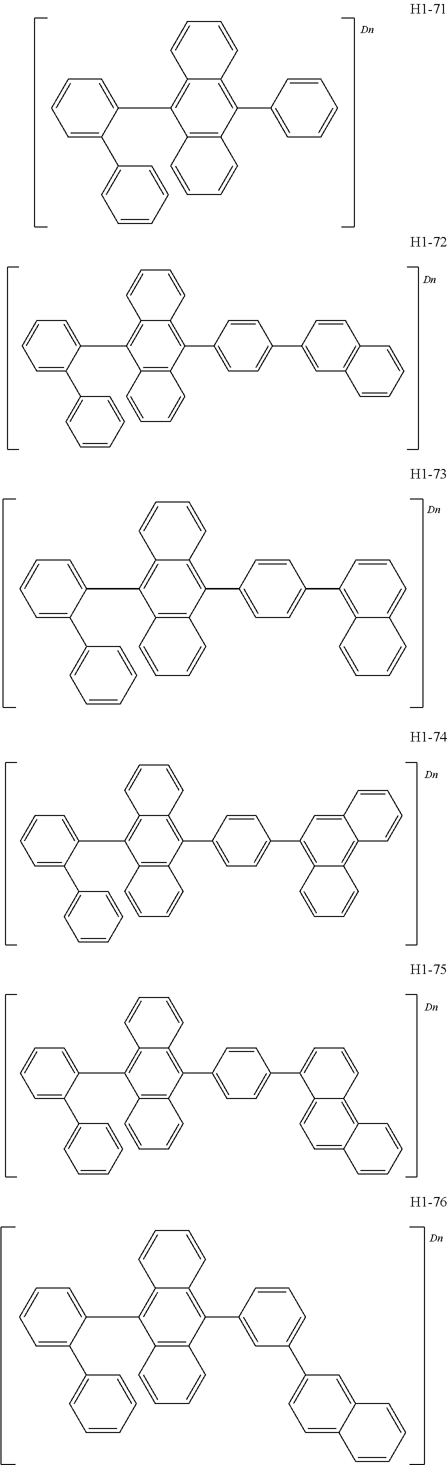 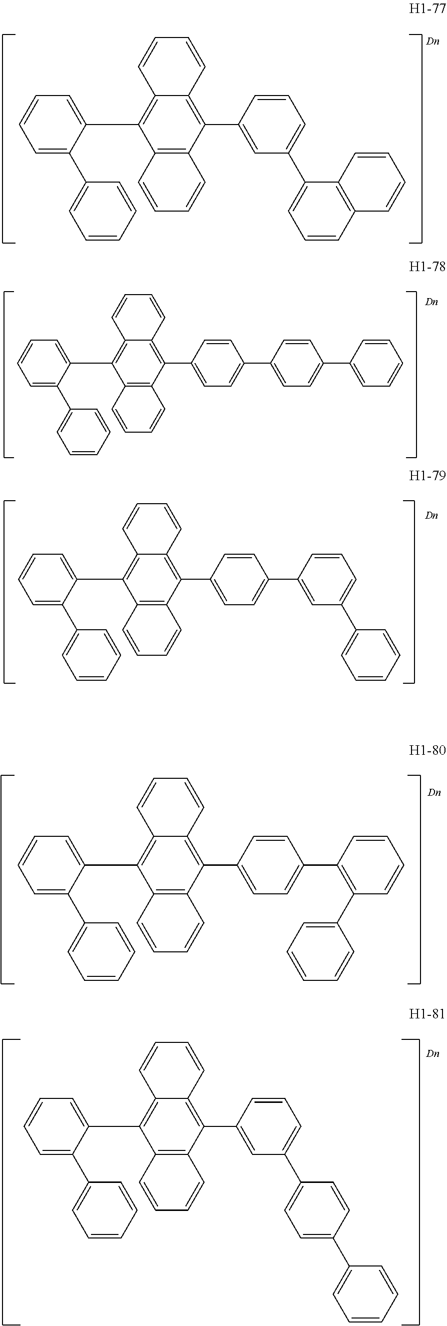 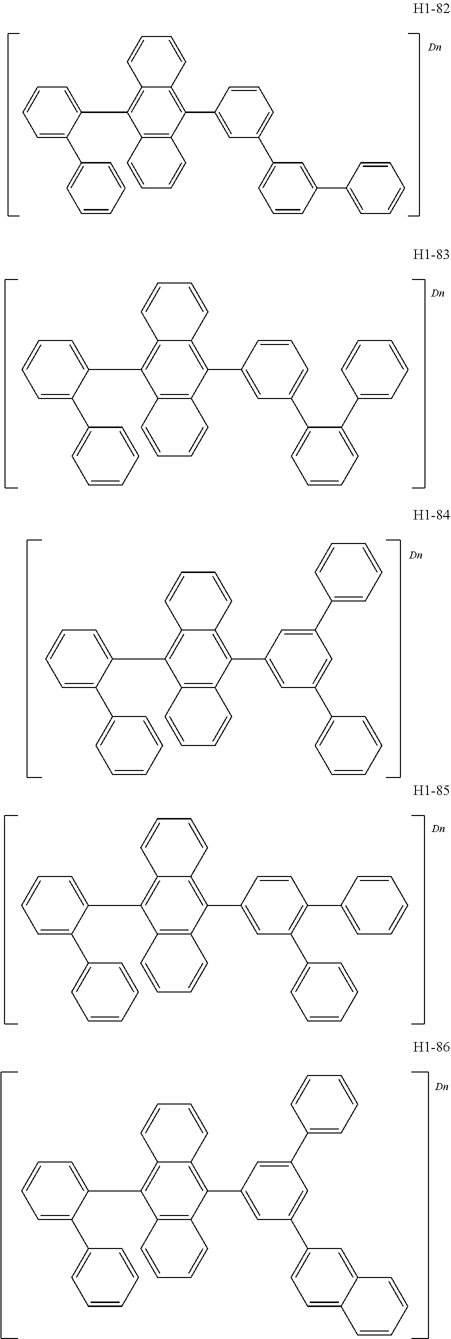 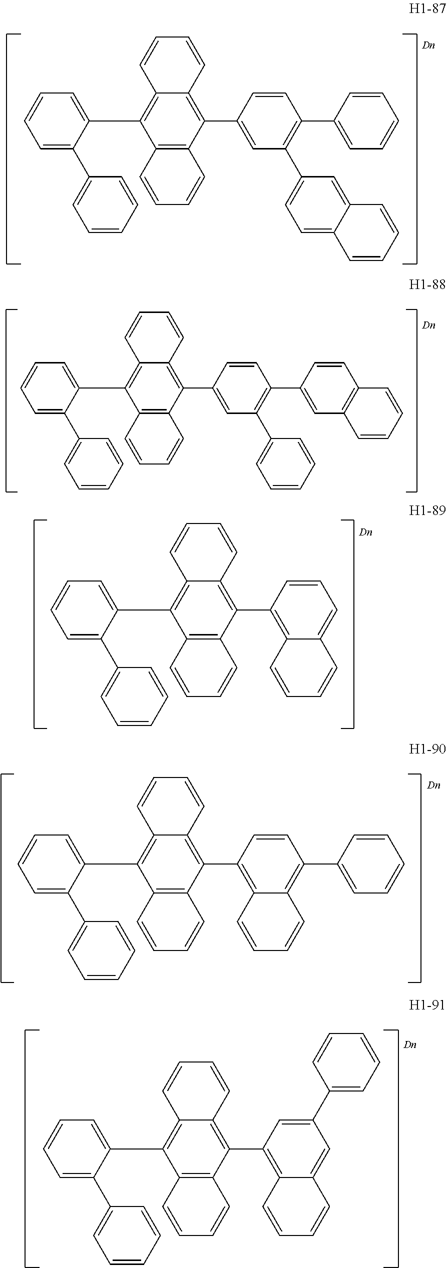 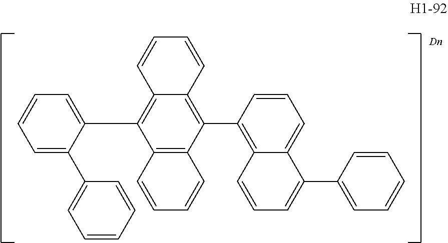 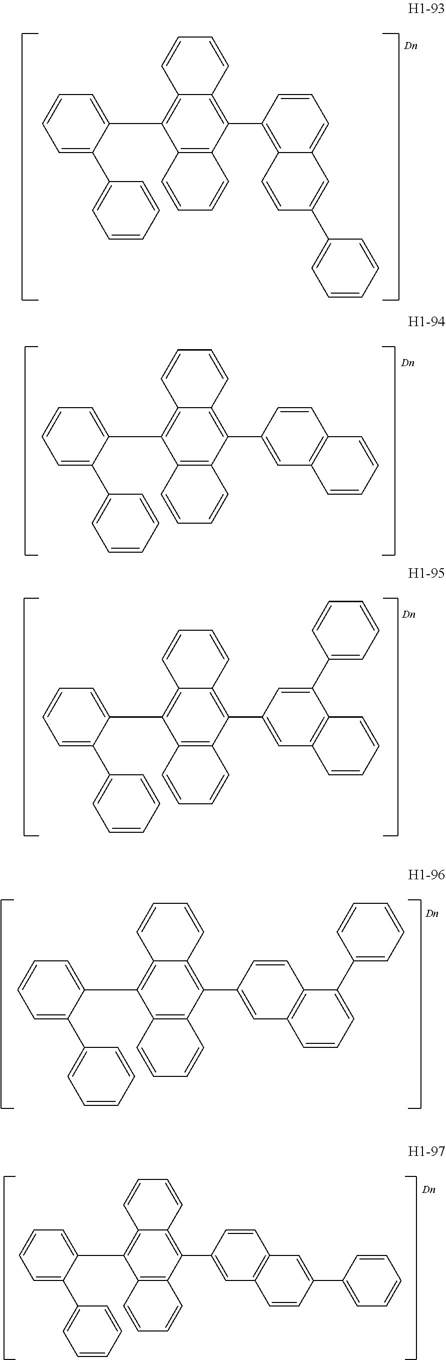 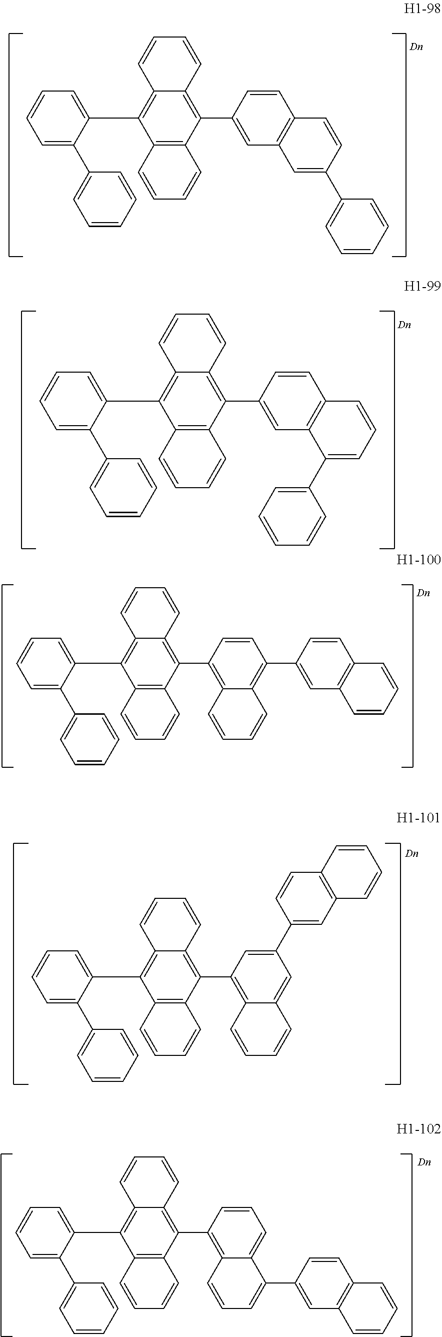 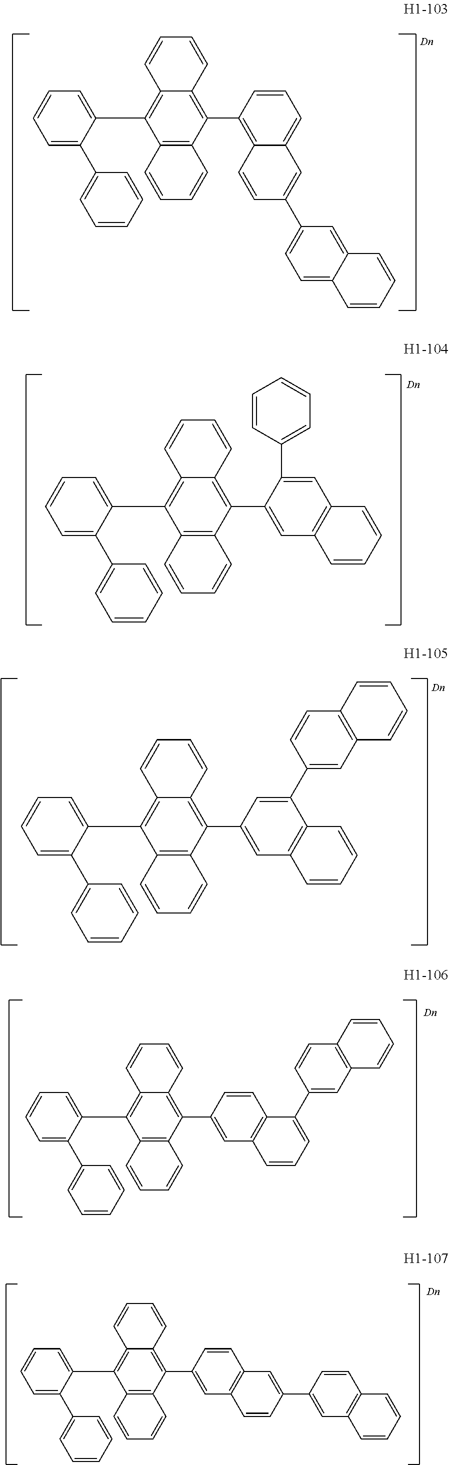 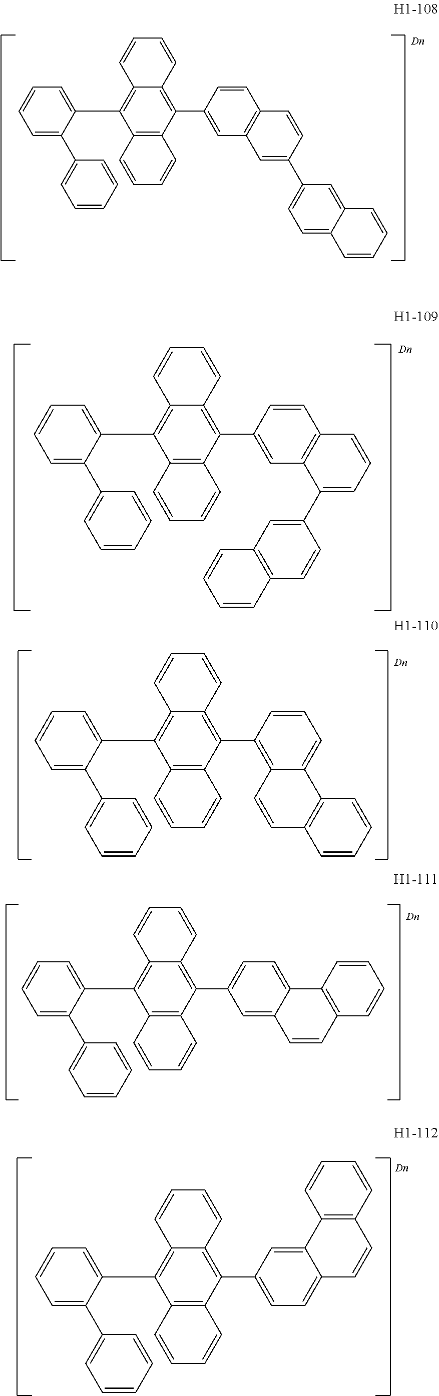 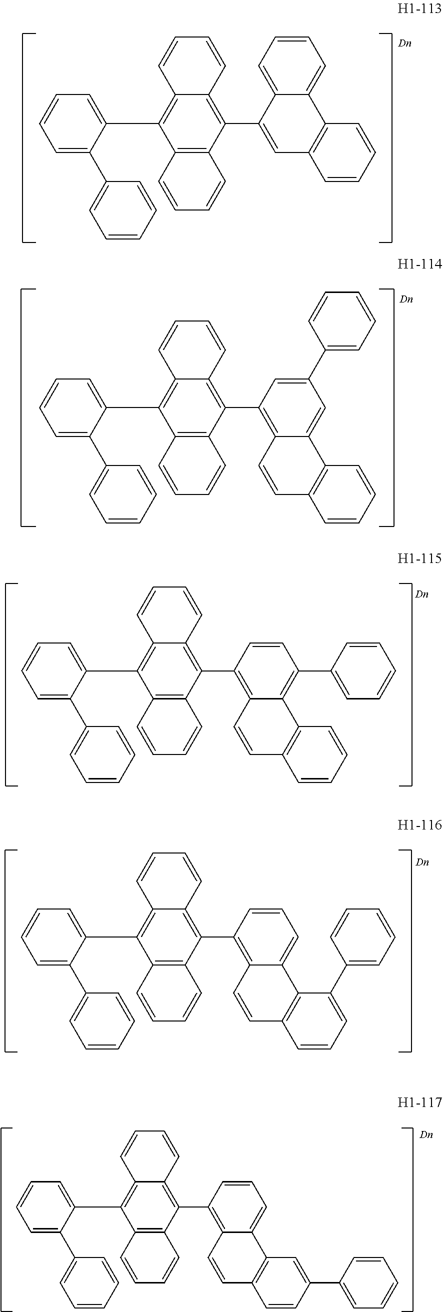 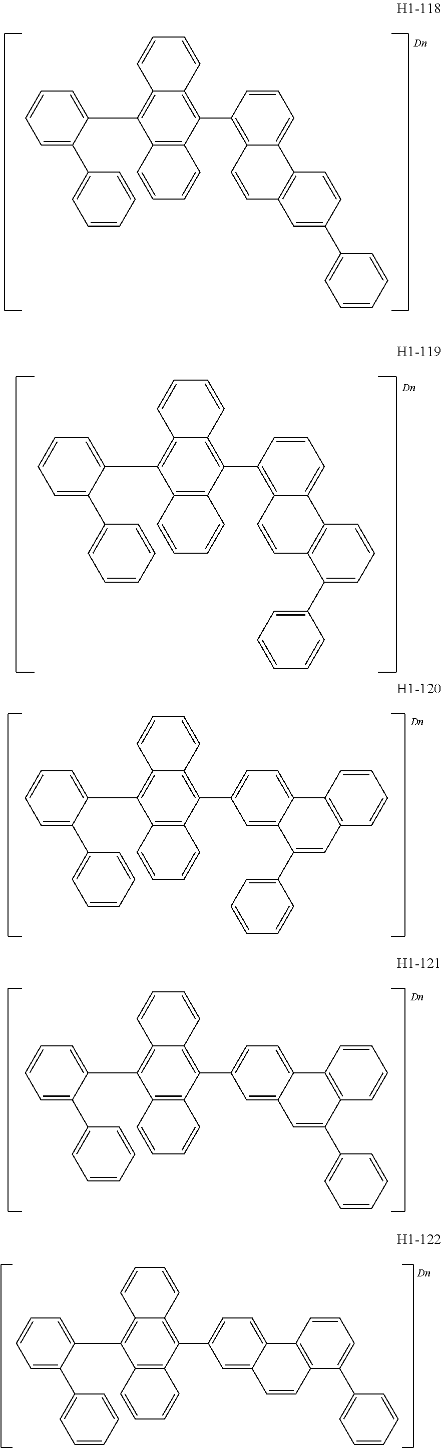 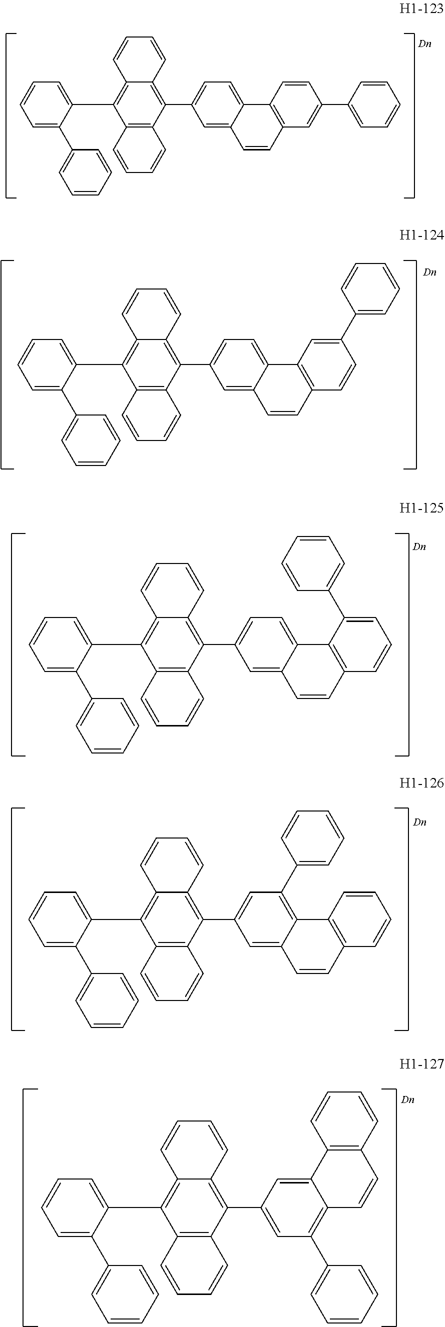 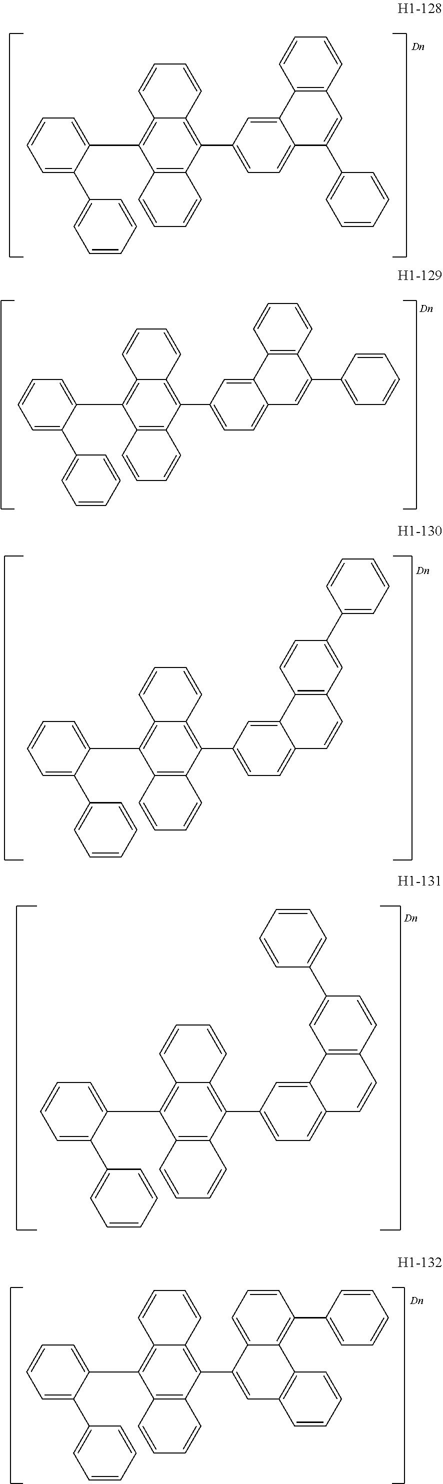 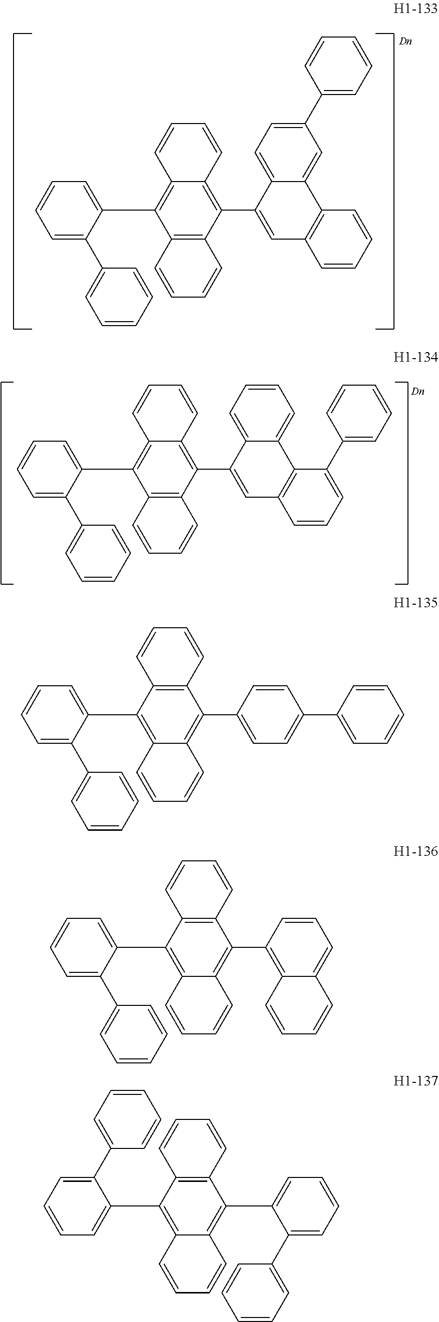 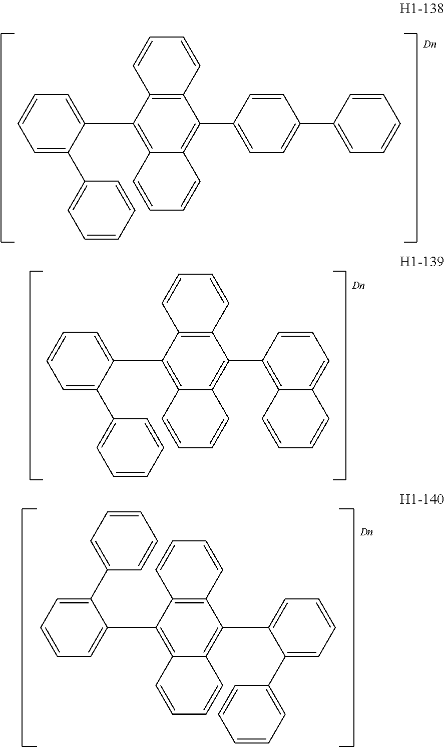 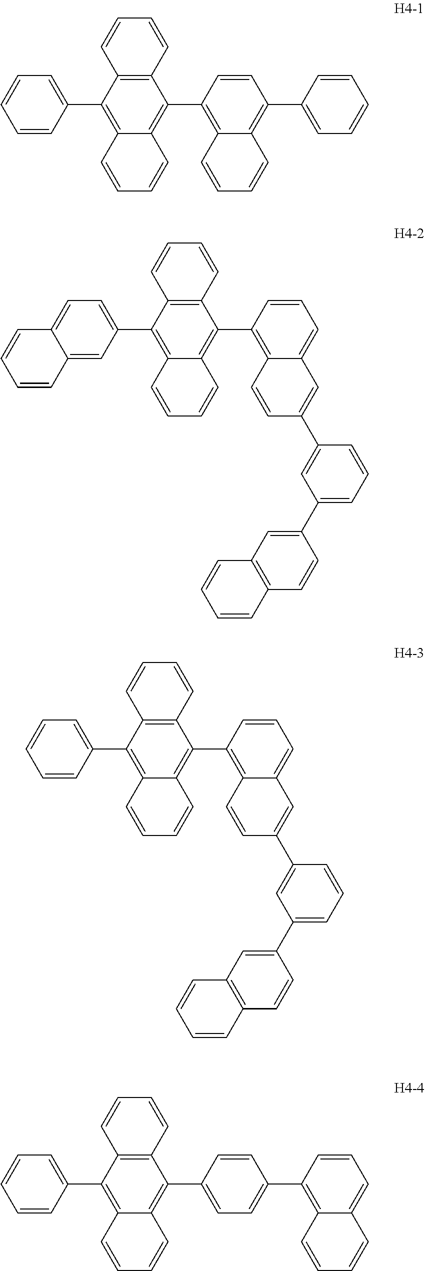 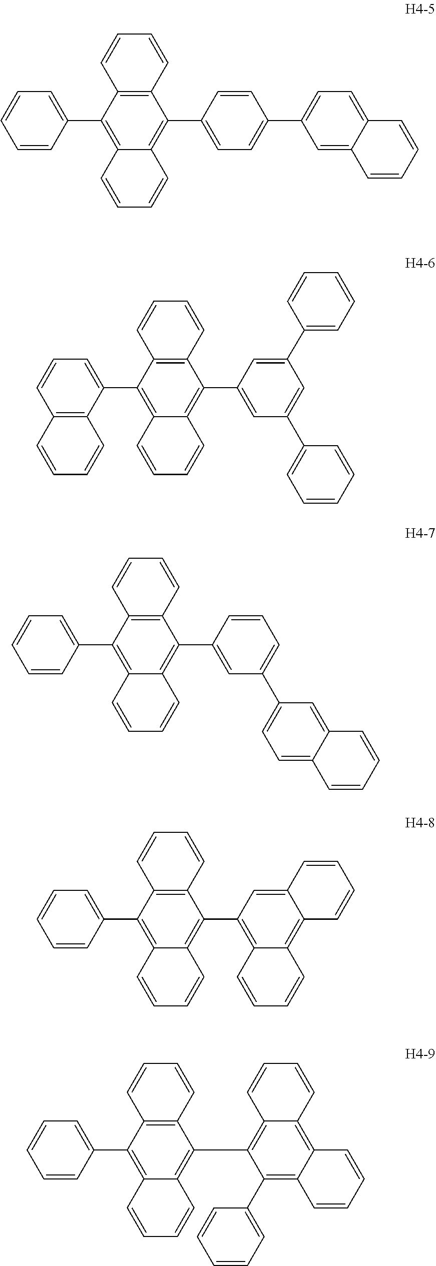 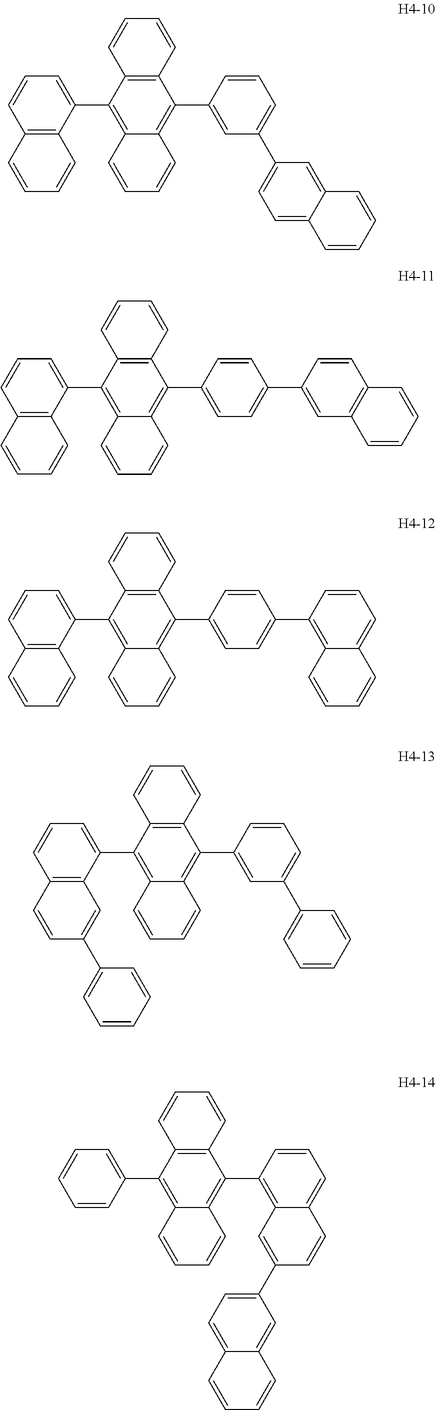 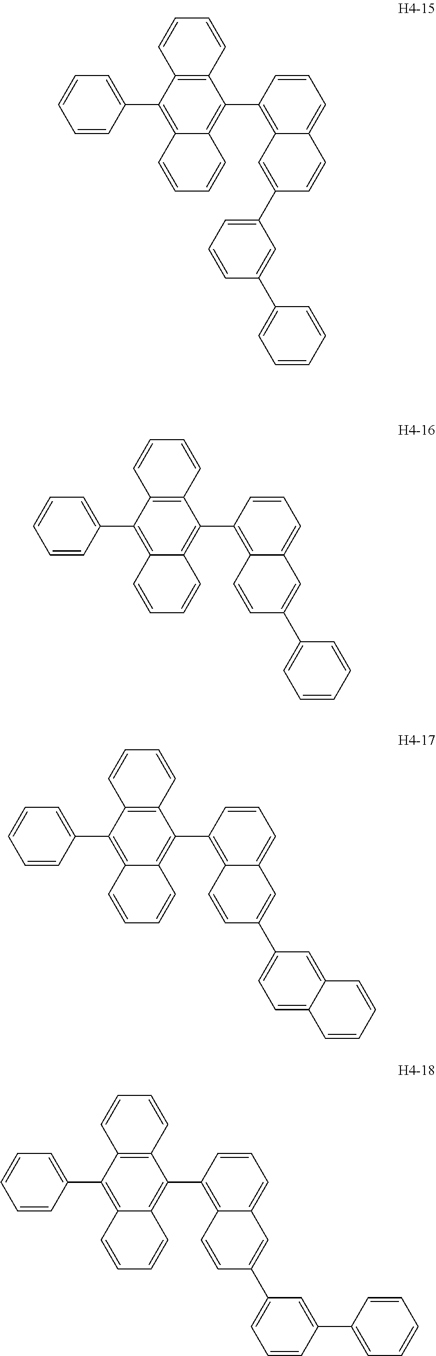 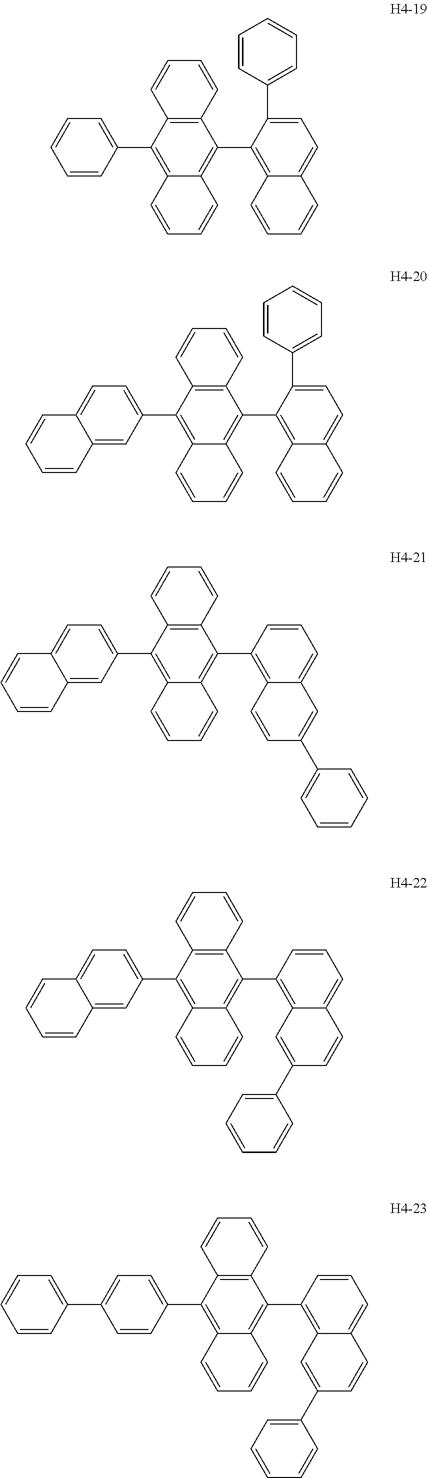 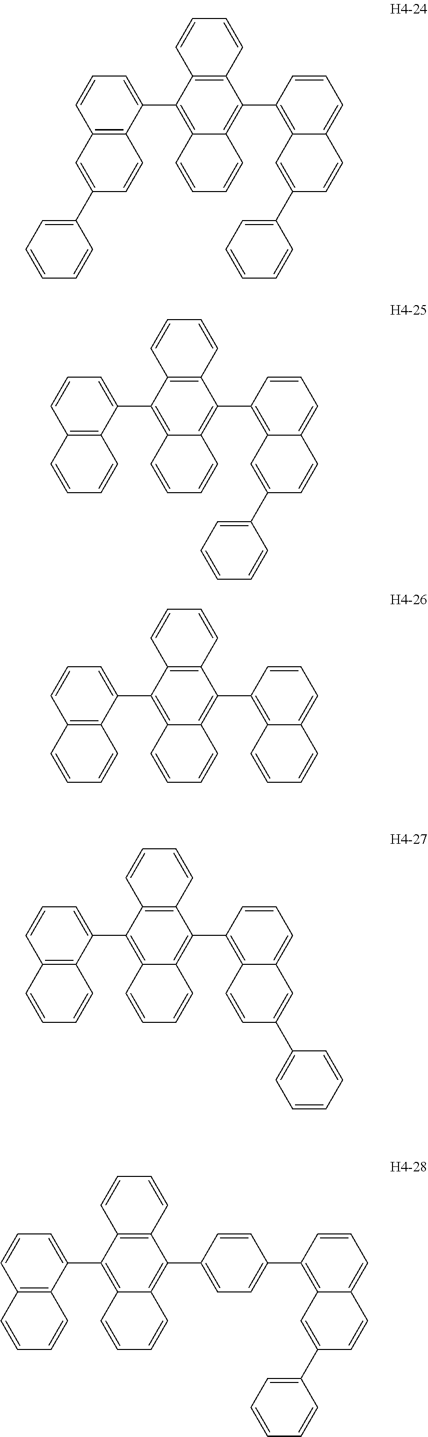 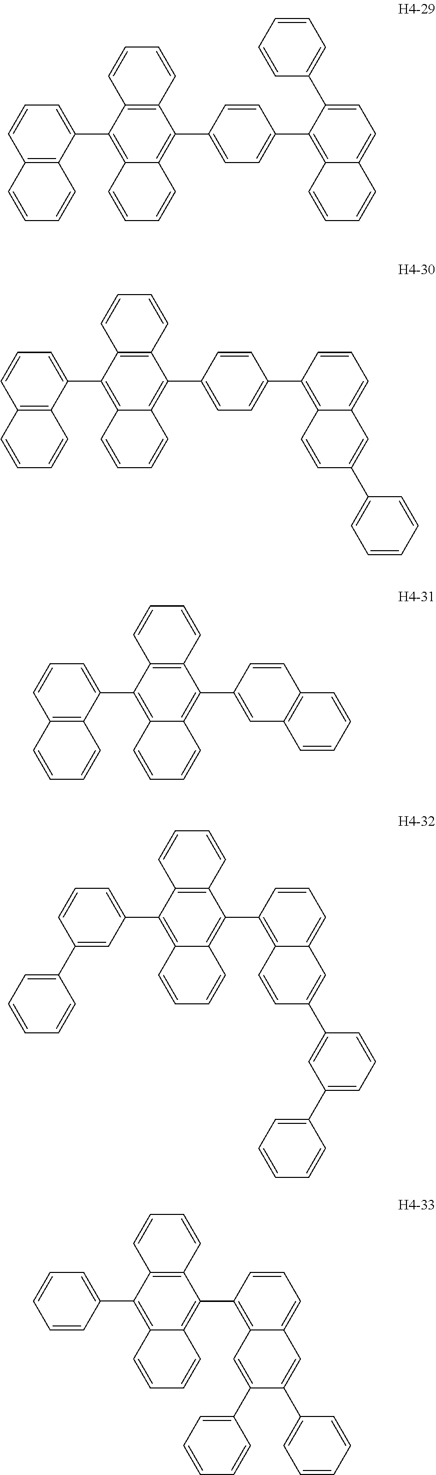 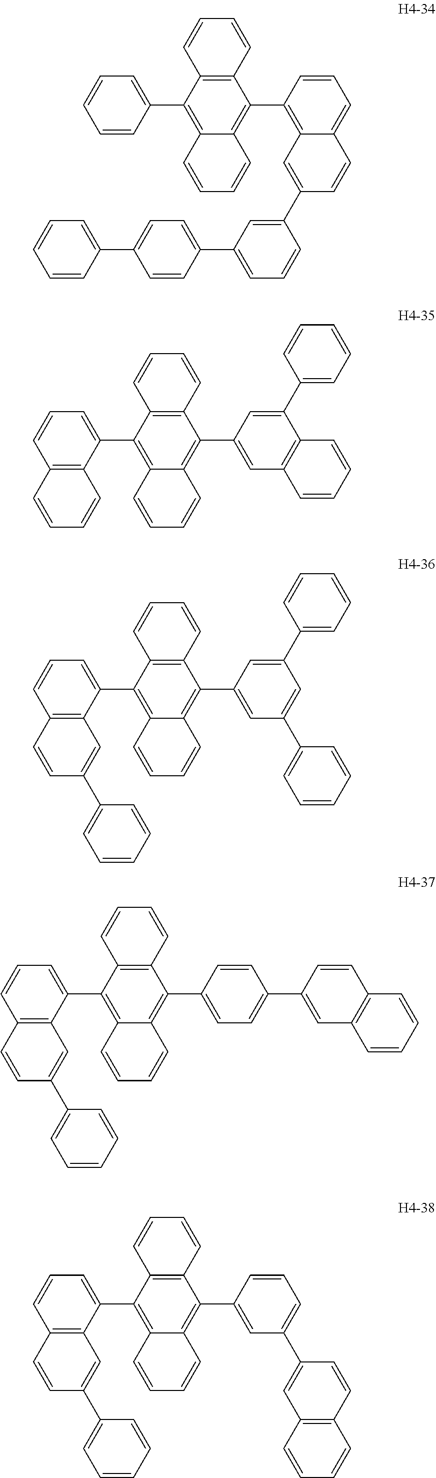 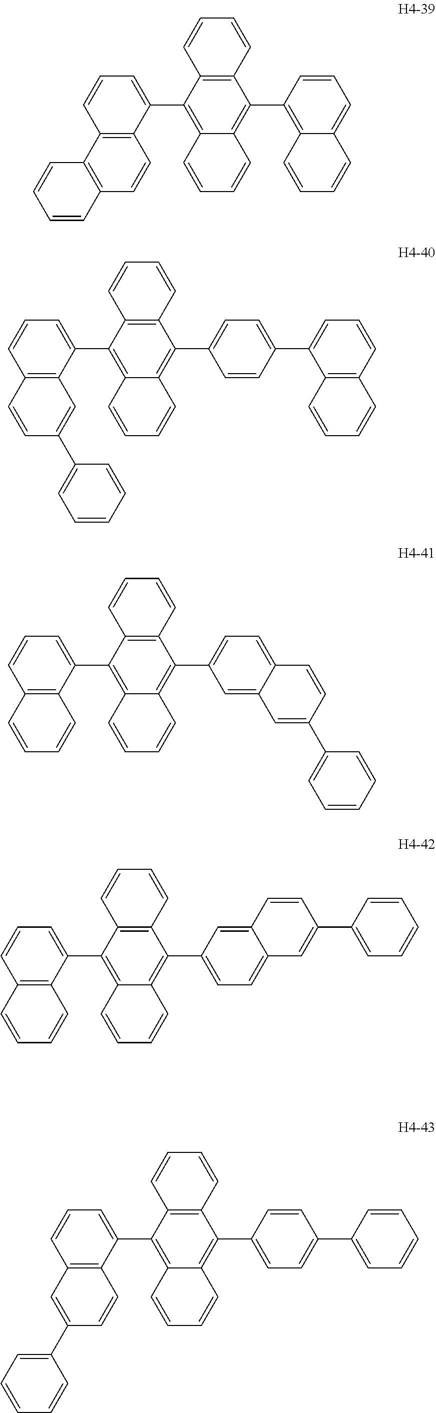 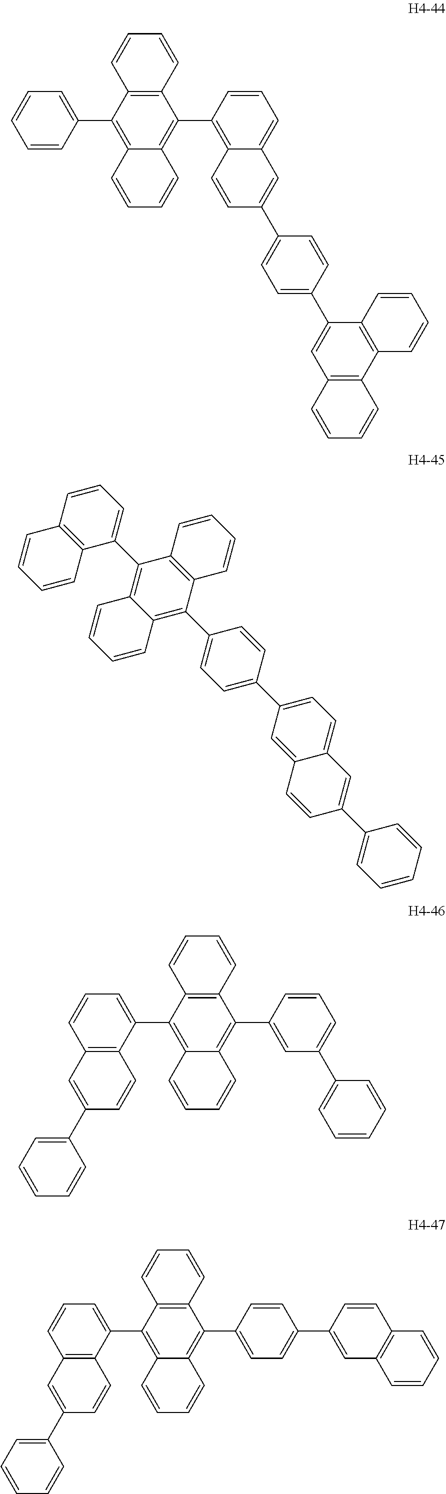 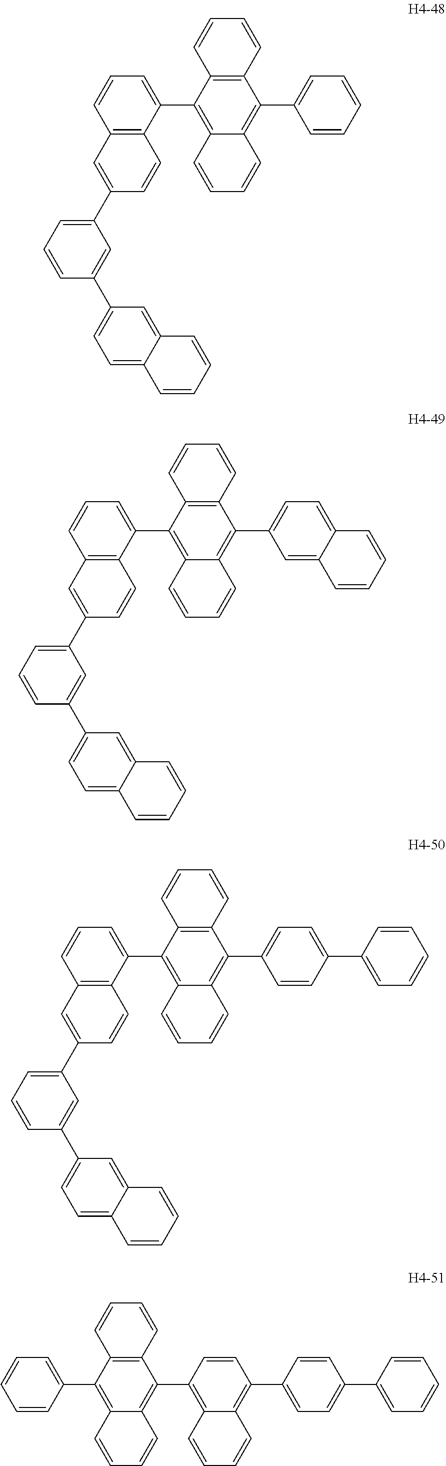 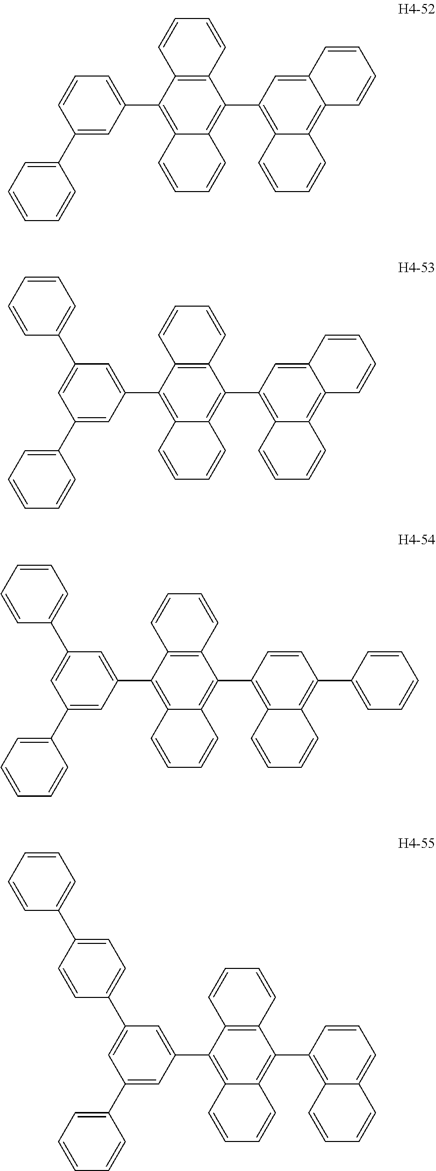 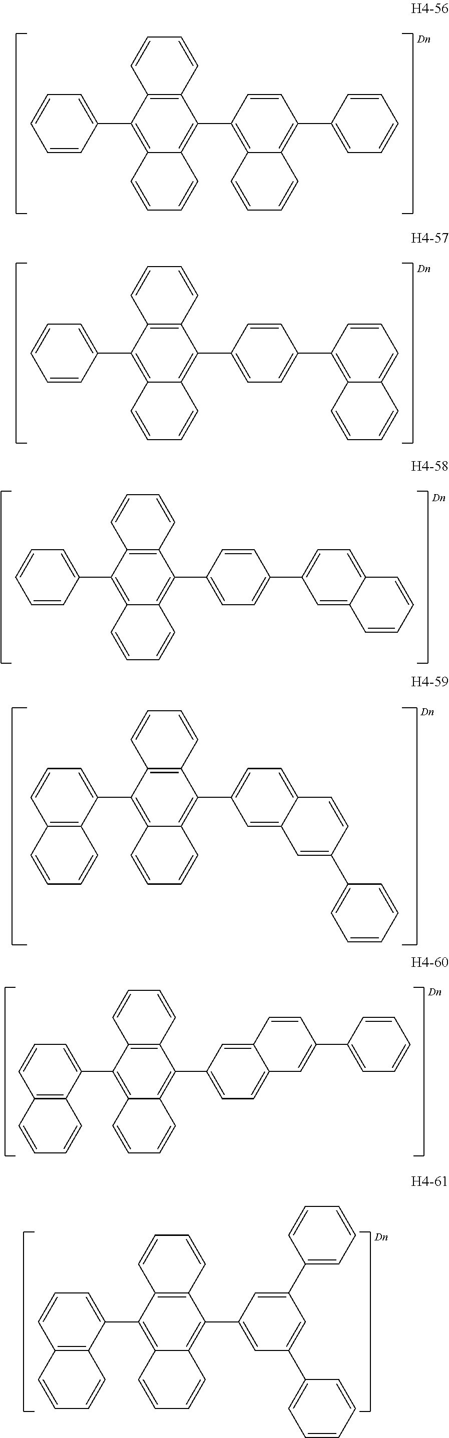 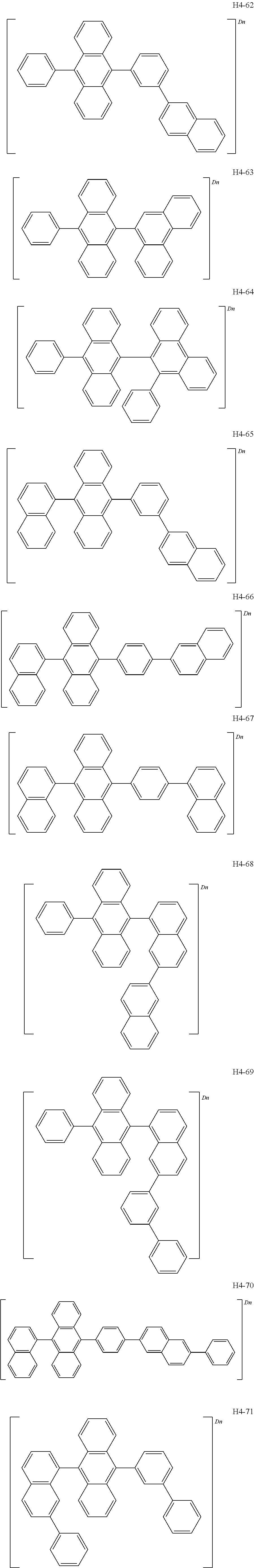 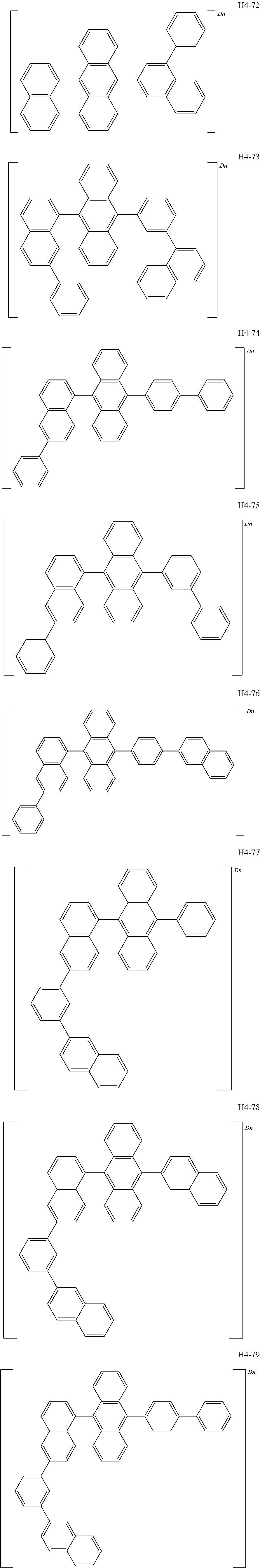 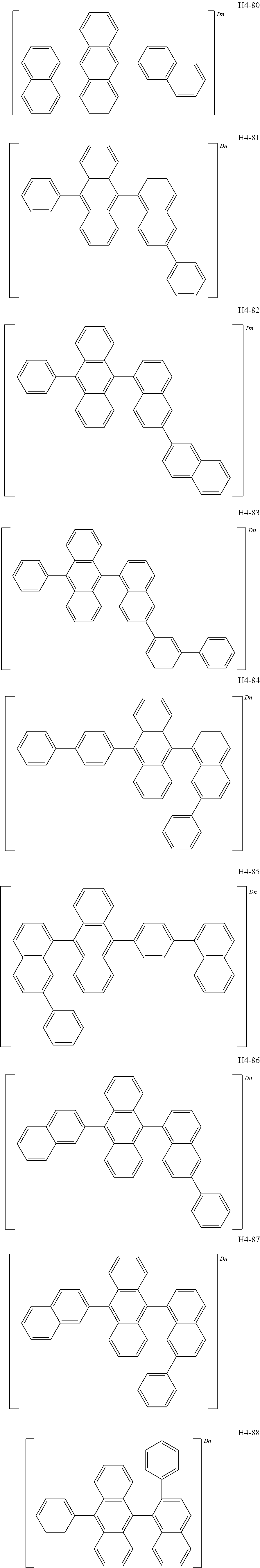 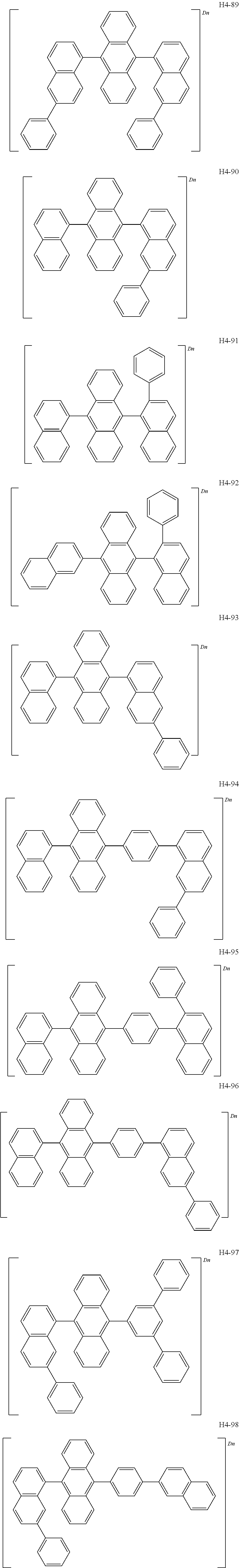 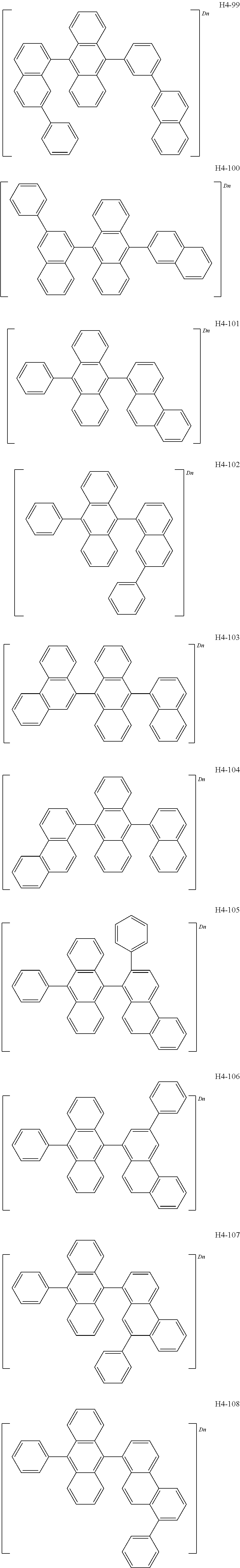 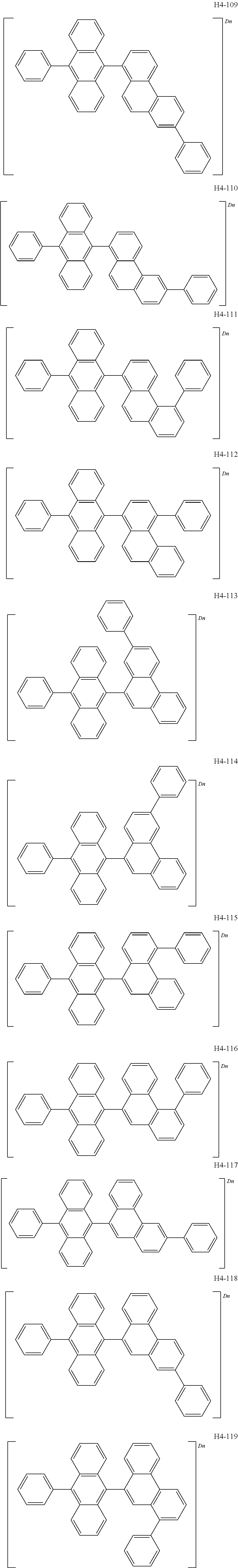 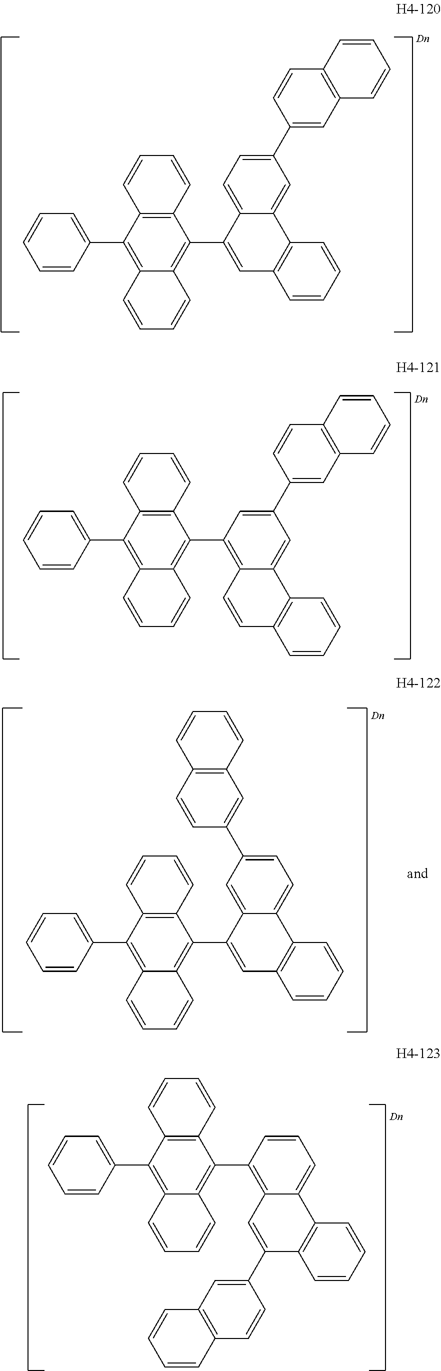
wherein Dₙ signifies that n hydrogens are replaced with deuterium, and n is an integer from 1 to the maximum number of hydrogens in the compound.

12. An organic electroluminescent device comprising an anode; a cathode; and at least one light-emitting layer between the anode and the cathode, wherein at least one layer of the light-emitting layers comprises the plurality of host materials according to claim 1.

13. The organic electroluminescent device according to claim 12, comprising an anode; a cathode; and at least two light-emitting layers between the anode and the cathode, wherein at least one layer of the light-emitting layers comprises the plurality of host materials according to claim 1.

14. An organic electroluminescent compound represented by the following Formula 11: 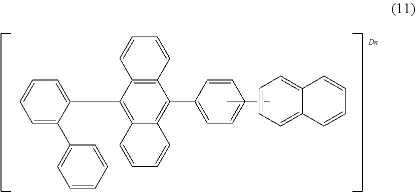
wherein Dₙ signifies that n hydrogens are replaced with deuterium, and n is an integer from 1 to 28.

15. The organic electroluminescent compound according to claim 14, wherein n is an integer from 8 to 27.

16. The organic electroluminescent compound according to claim 14, wherein the compound represented by Formula 11 is selected from the following compounds:

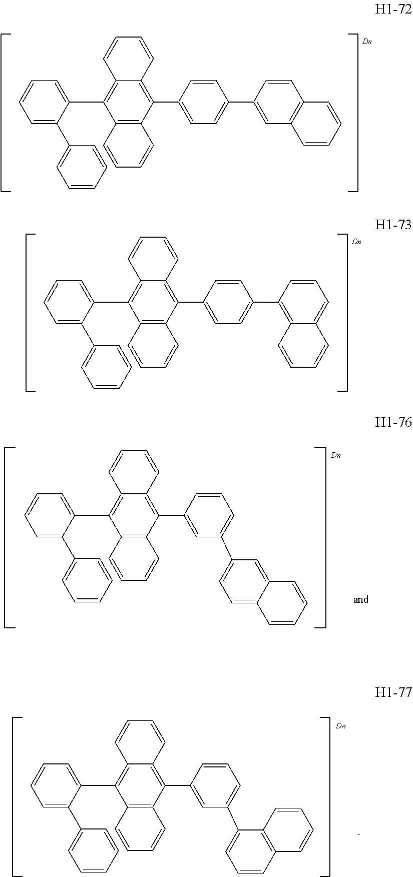

17. An organic electroluminescent device comprising an anode; a cathode; and at least one light-emitting layer between the anode and the cathode, wherein at least one layer of the light-emitting layers comprises the organic electroluminescent compound according to claim 14.

18. The organic electroluminescent device according to claim 17, comprising an anode; a cathode; and at least two light-emitting layers between the anode and the cathode, wherein at least one layer of the light-emitting layers comprises the organic electroluminescent compound according to claim 14.

19. An organic electroluminescent device comprising an anode; a cathode; and at least two light-emitting layers between the anode and the cathode, wherein at least one layer of the light-emitting layers comprises an organic electroluminescent compound represented by the following Formula 1 as a host material: 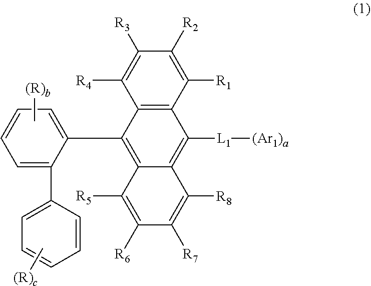
in Formula 1,
L₁ represents a single bond, a phenylene unsubstituted or substituted with deuterium, a naphthylene unsubstituted or substituted with deuterium, or a phenanthrenylene unsubstituted or substituted with deuterium;
Ar₁ each independently represents a substituted or unsubstituted (C₆-C₃₀)aryl, or a substituted or unsubstituted (3- to 30-membered)heteroaryl;
R and R₁ to R₈ each independently represent hydrogen or deuterium; and
a represents an integer of 1 or 2, b represents an integer of 4, and c represents an integer of 5; and each Ar₁ may be the same as or different from each other;
with a proviso that Formula 1 is not represented as

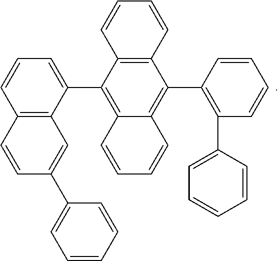 [SMILES: c1ccc(-c2ccc3cccc(-c4c5ccccc5c(-c5ccccc5-c5ccccc5)c5ccccc45)c3c2)cc1]

20. The organic electroluminescent device according to claim 19, wherein the compound represented by Formula 1 is selected from the following compounds: 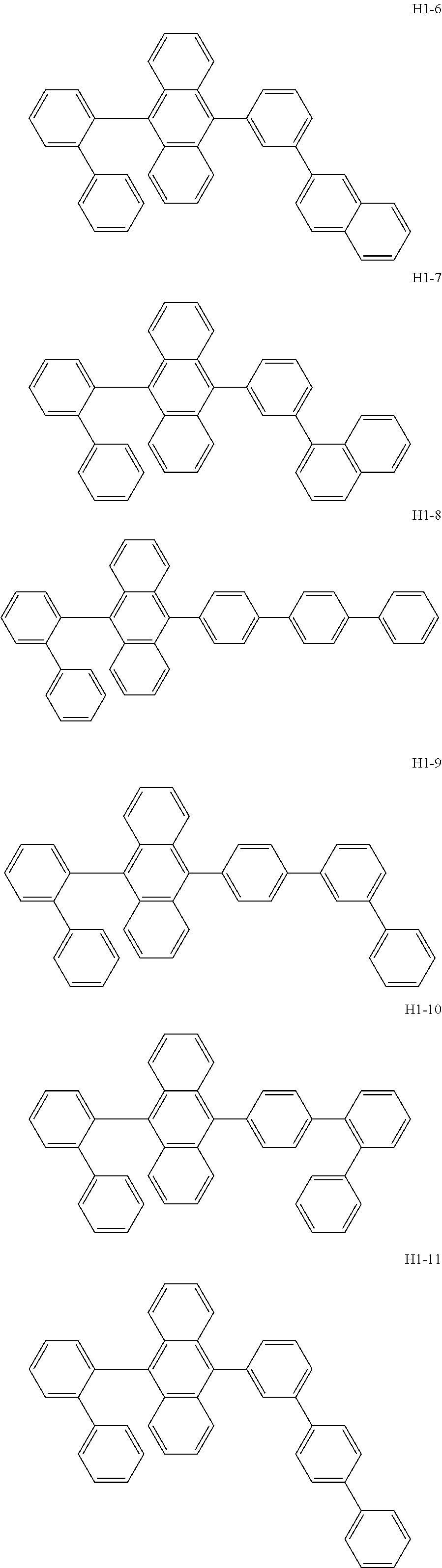 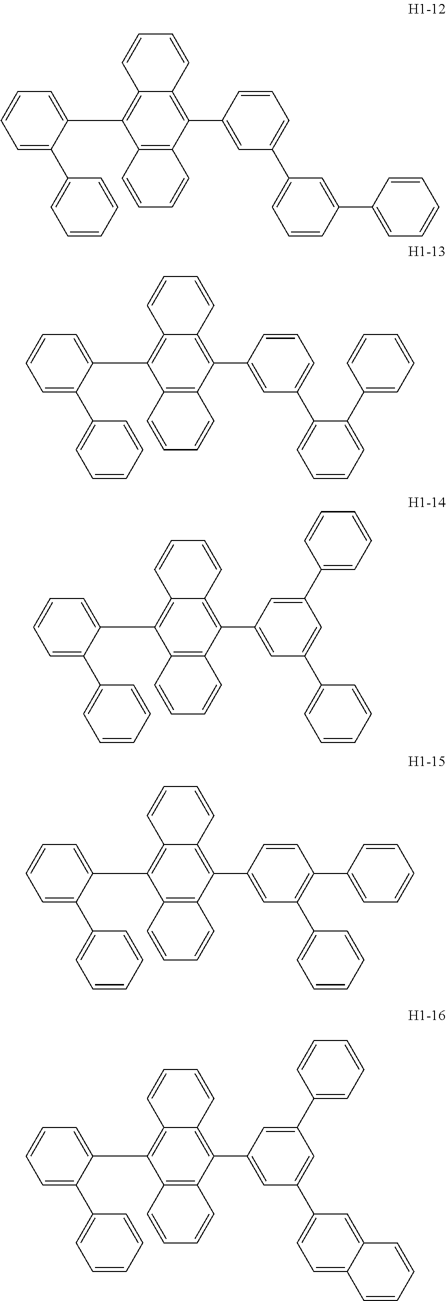 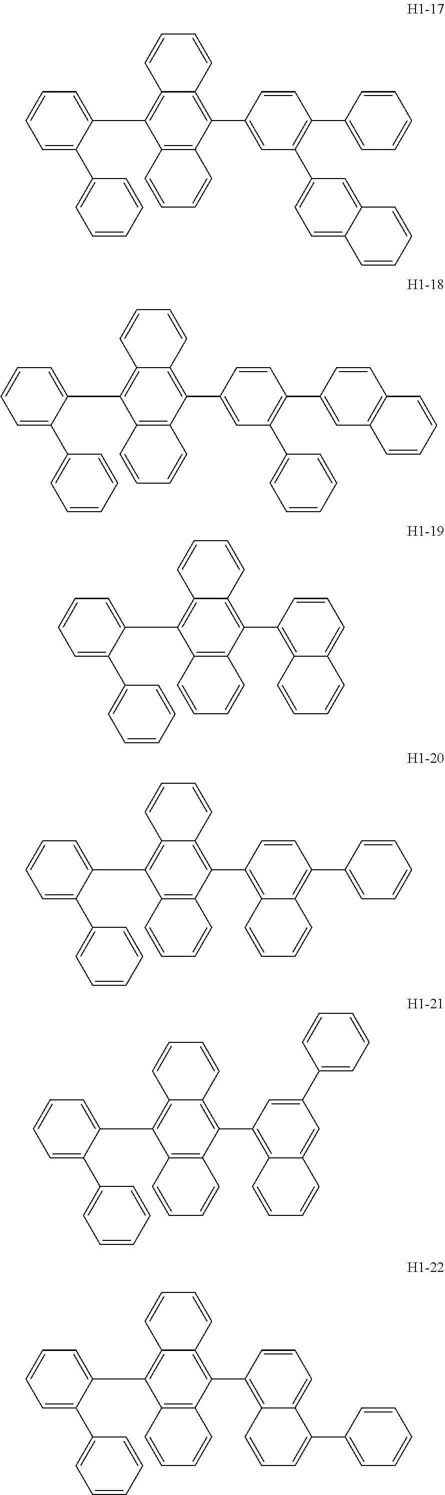 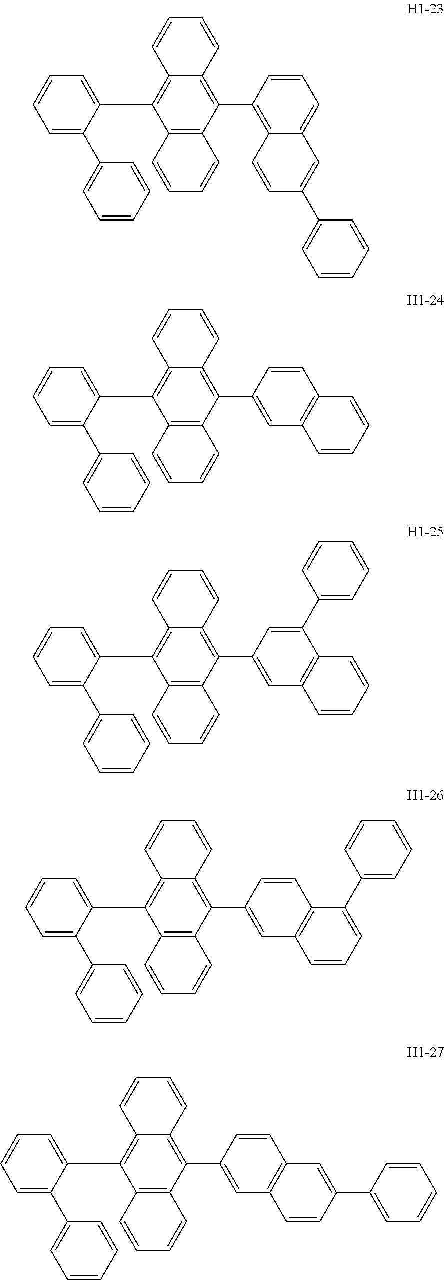 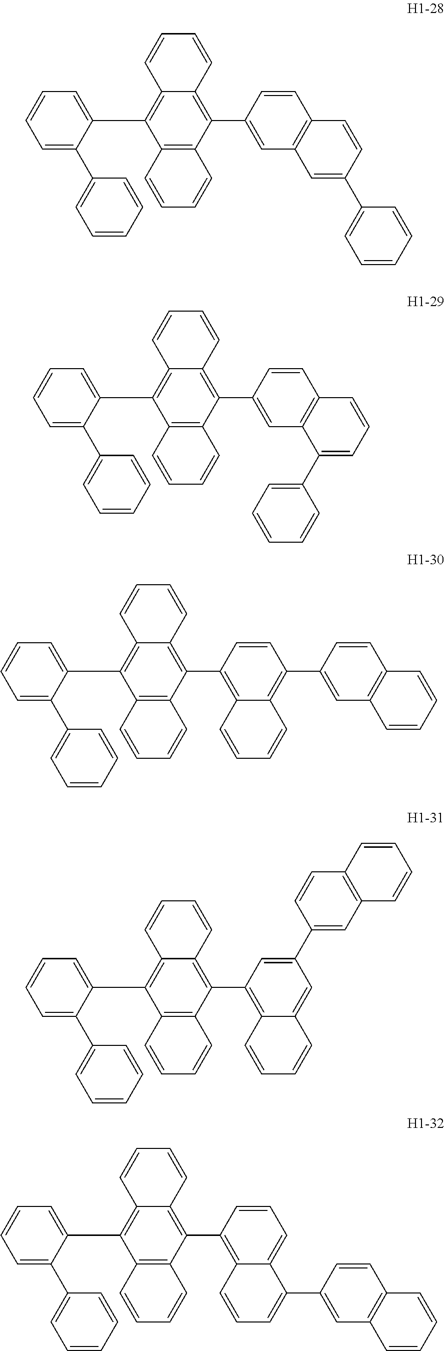 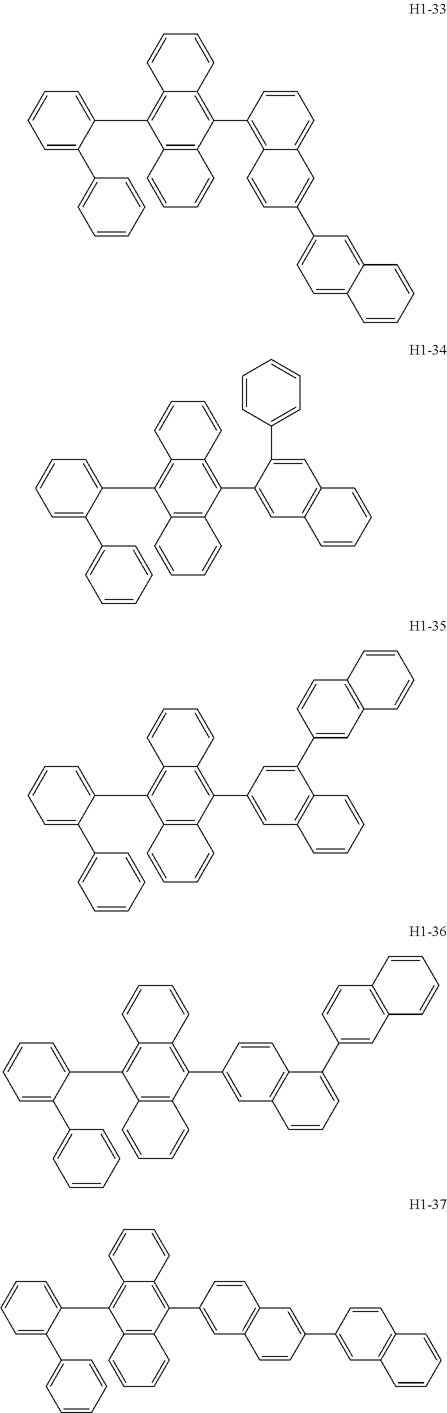 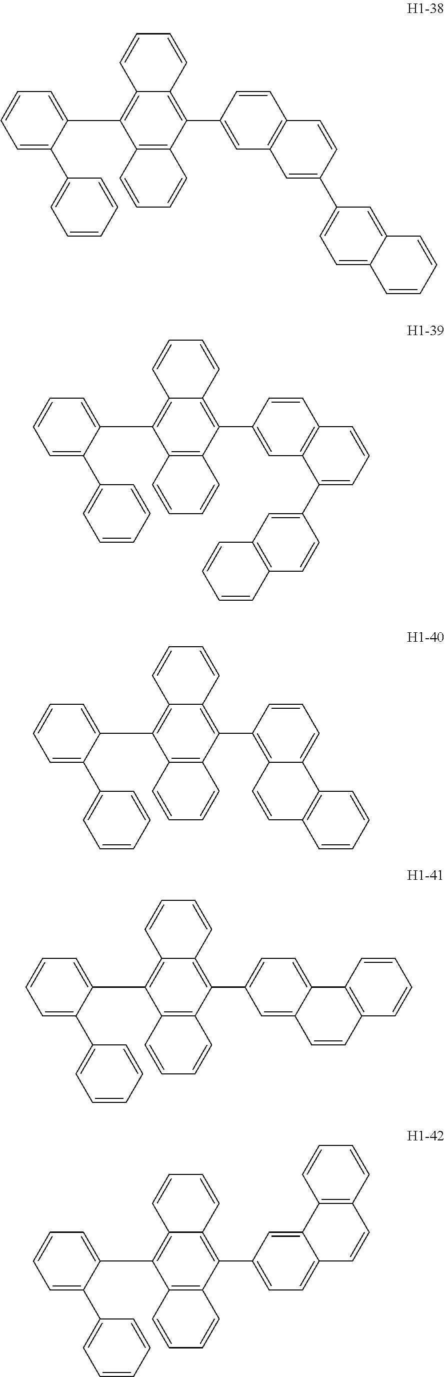 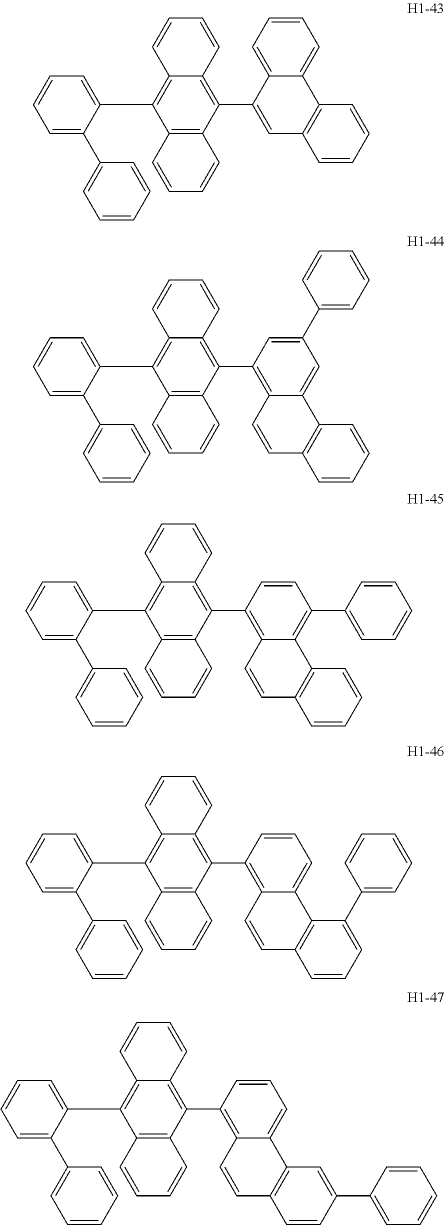 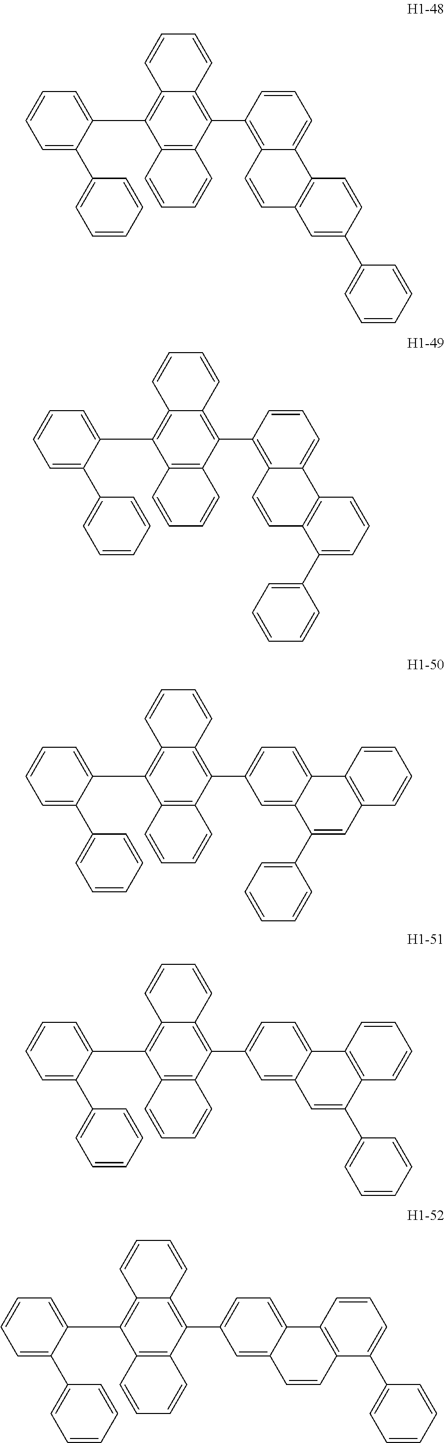 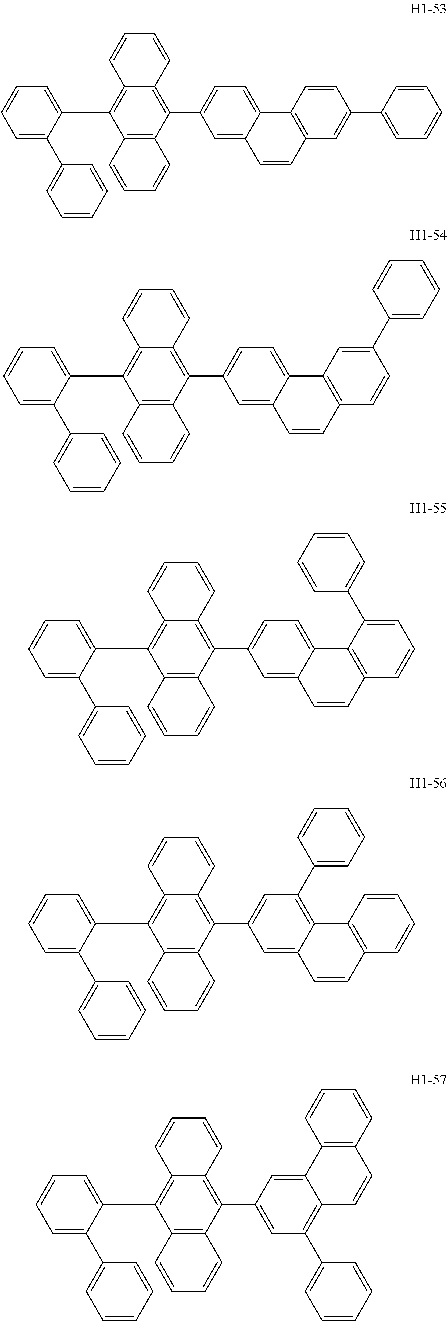 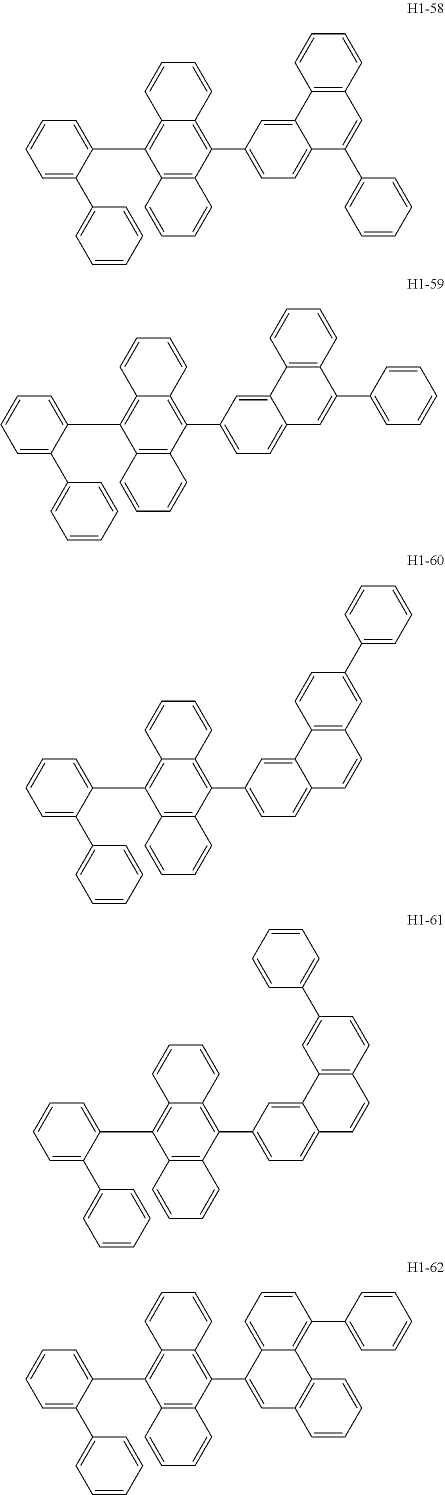 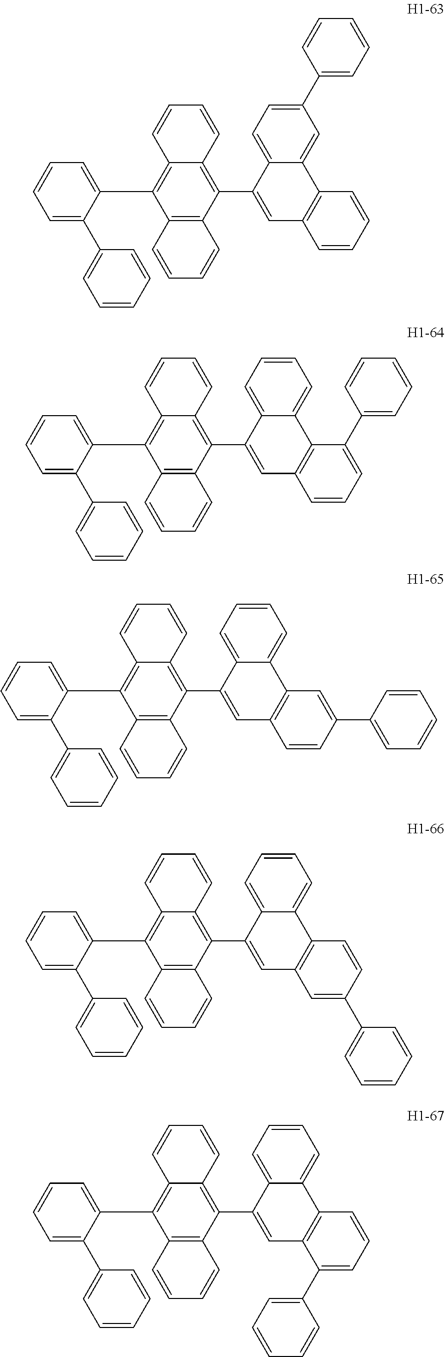 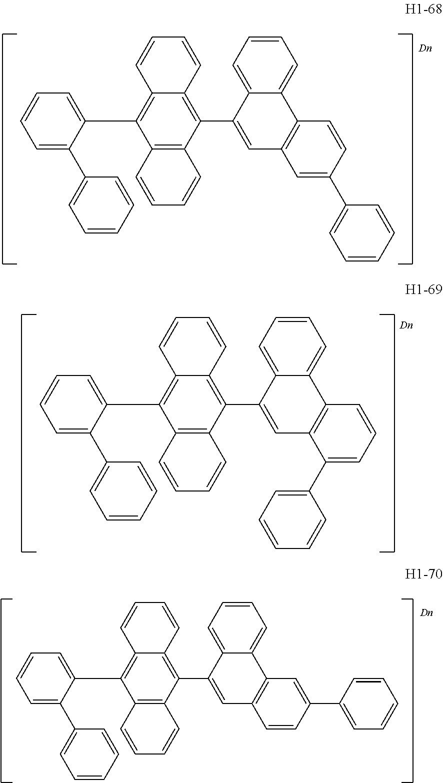 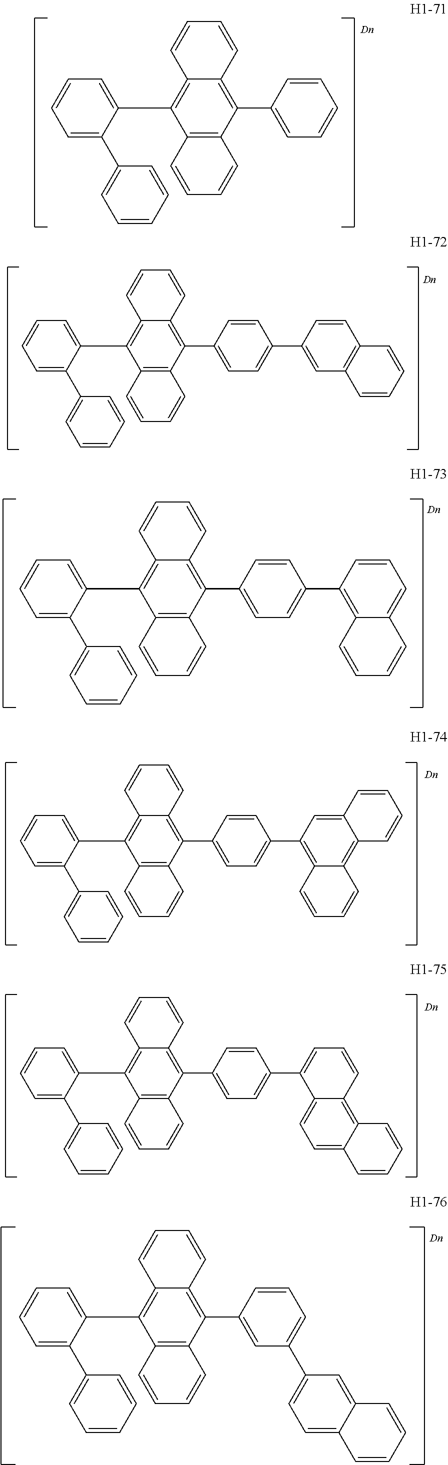 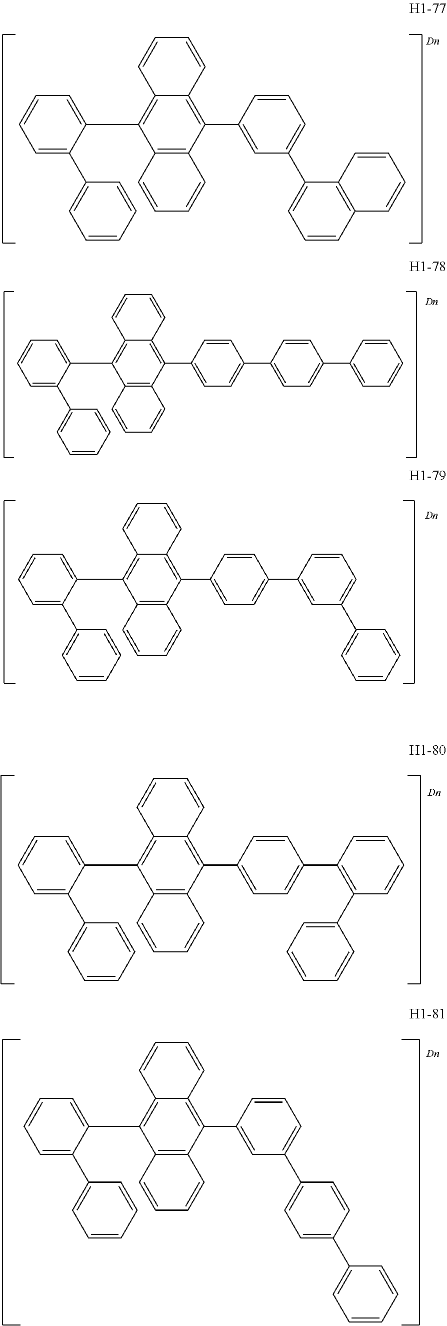 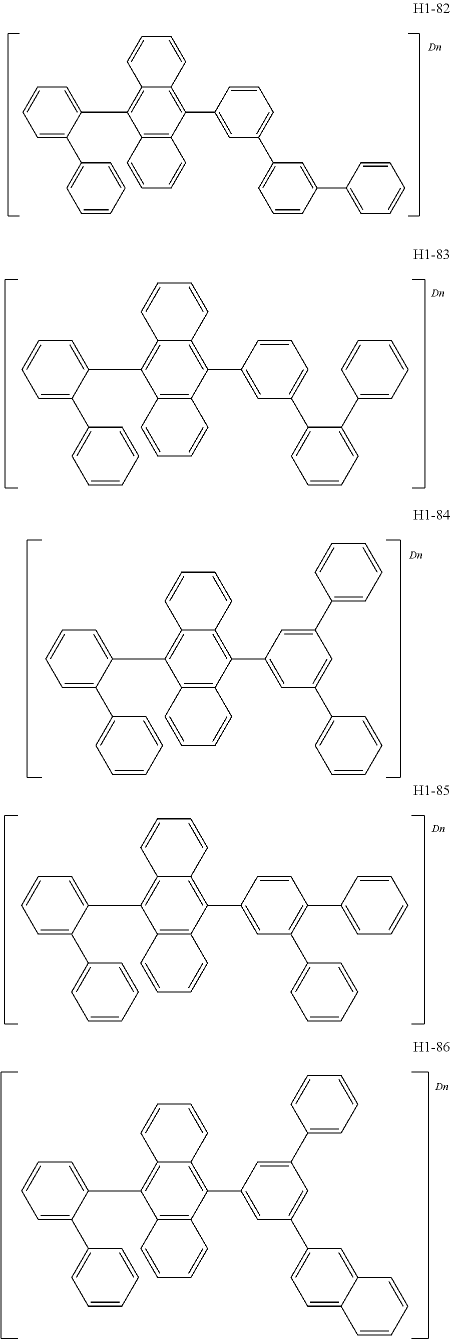 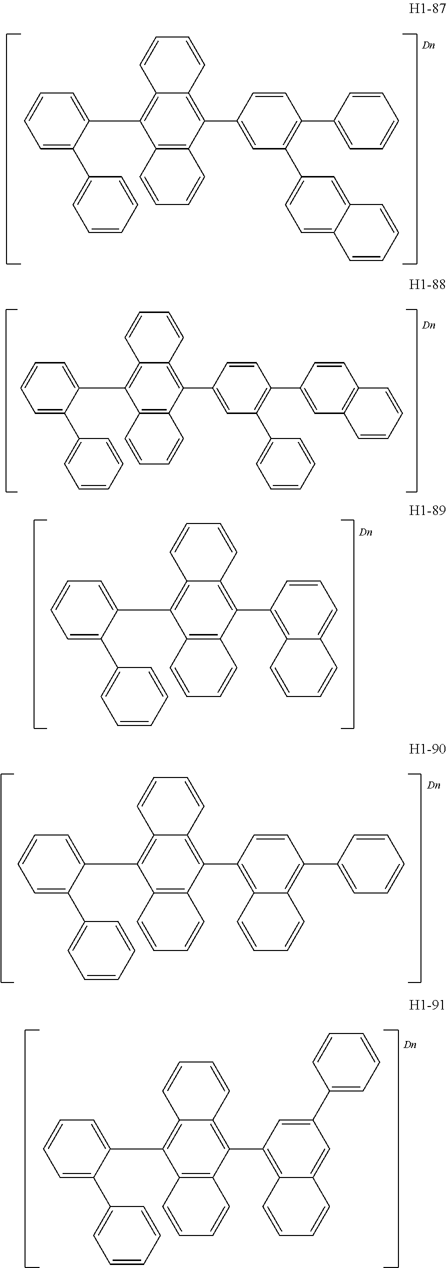 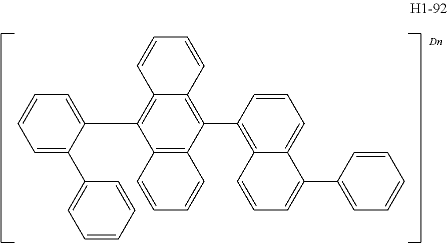 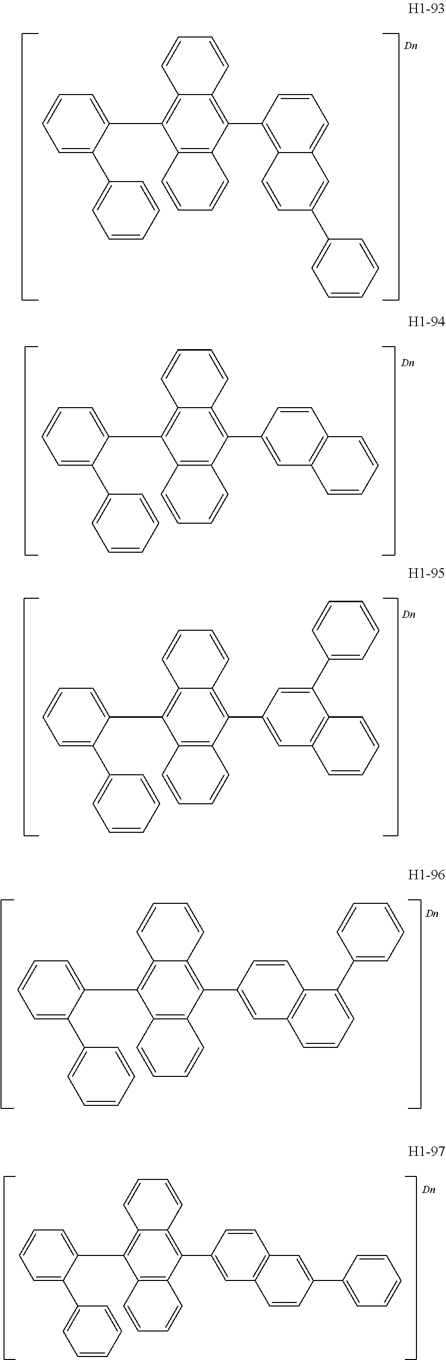 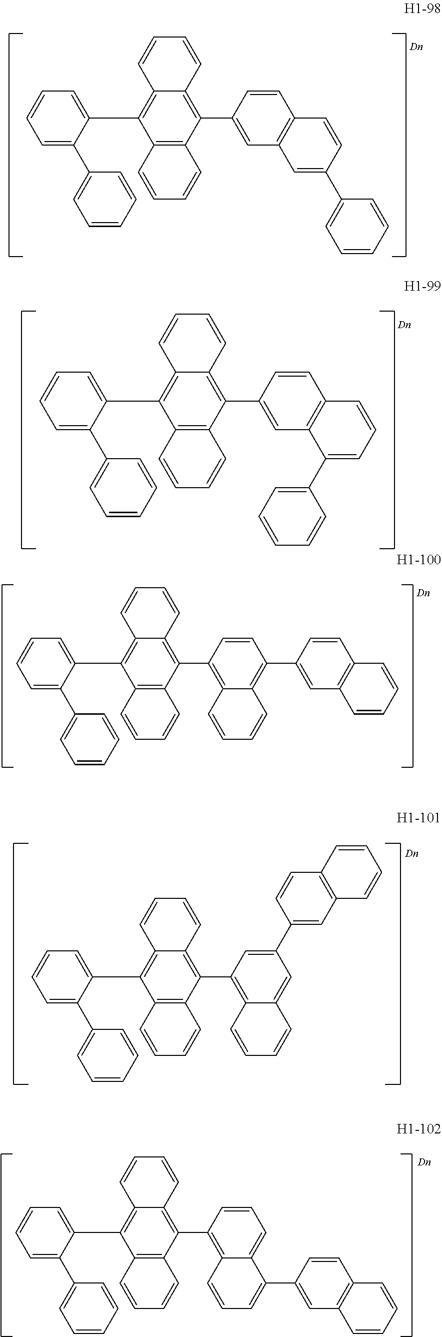 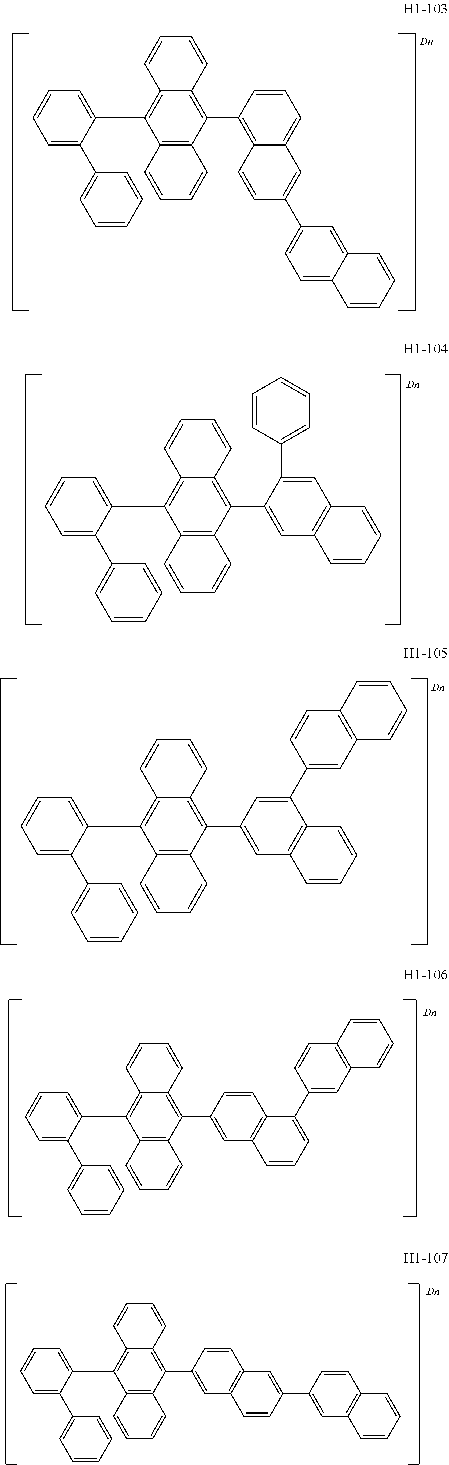 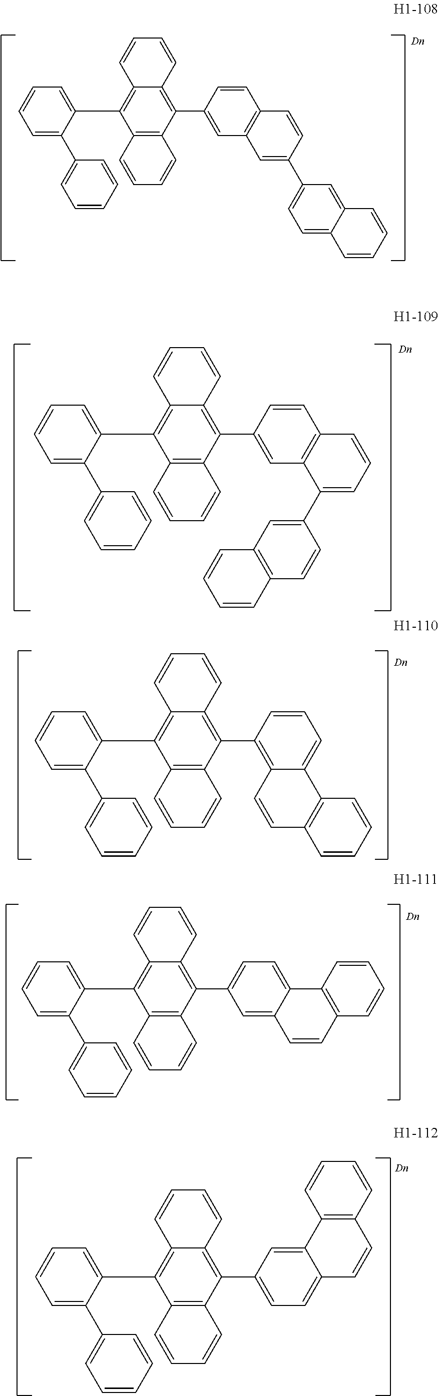 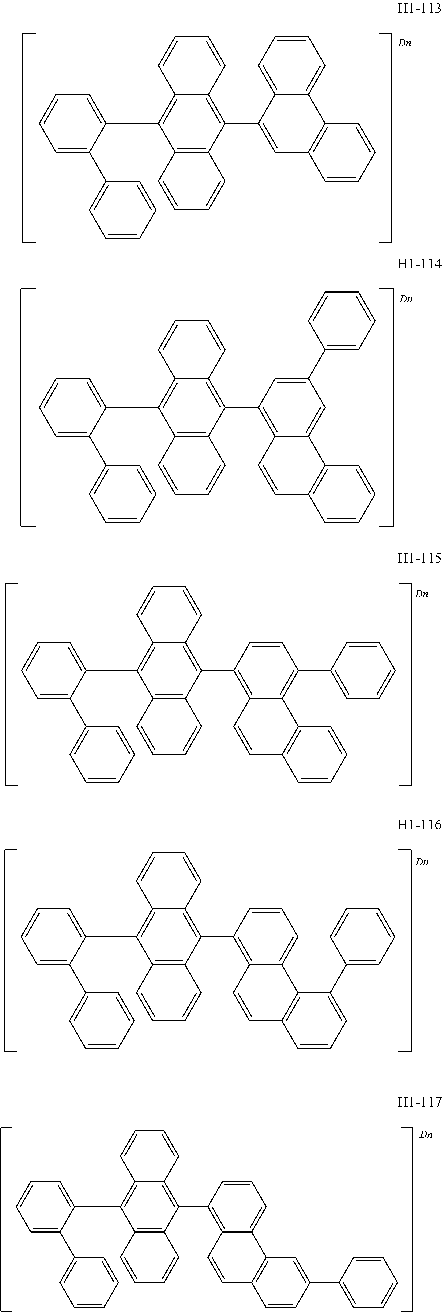 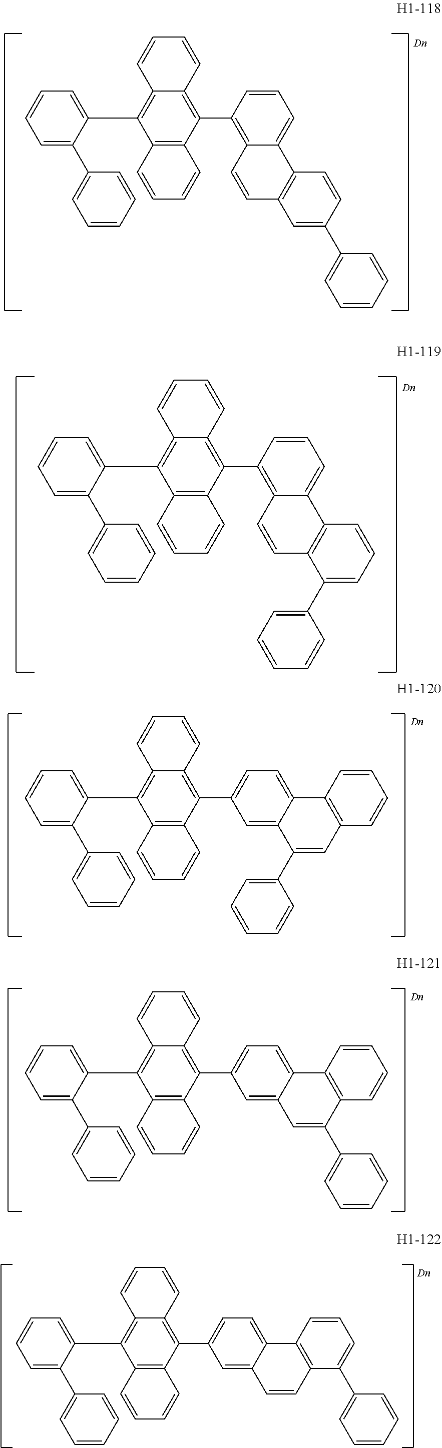 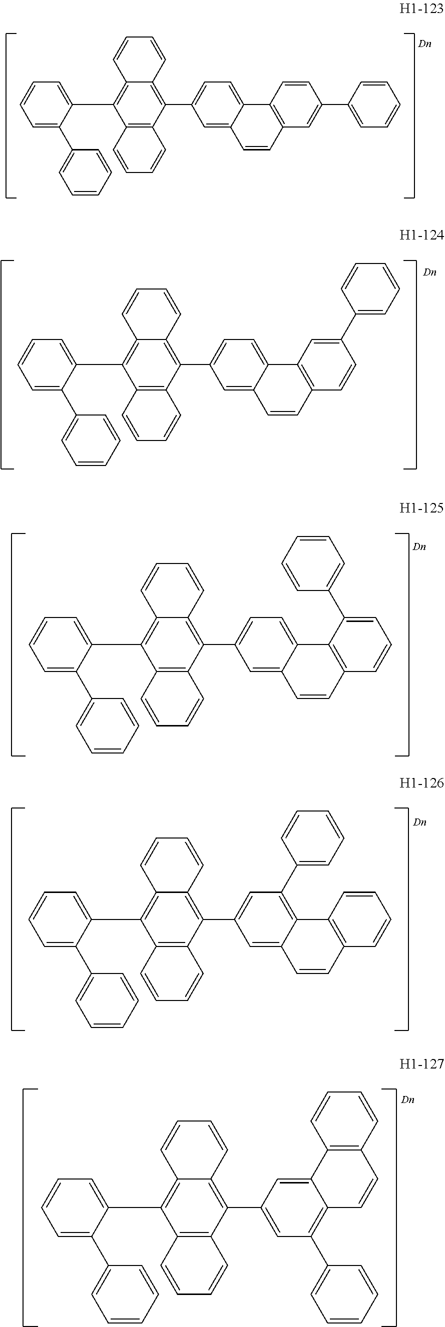 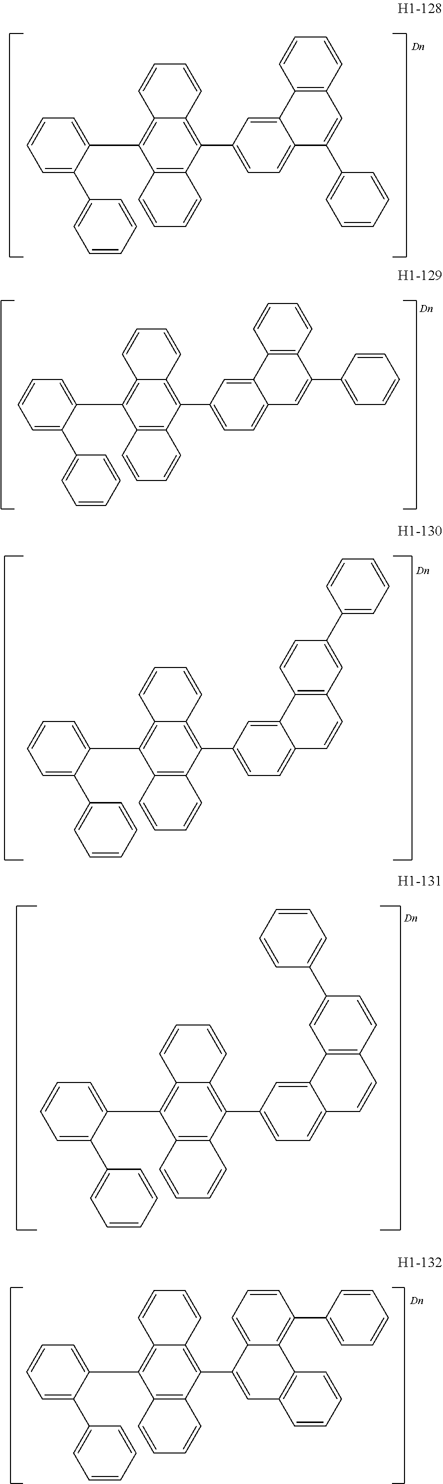 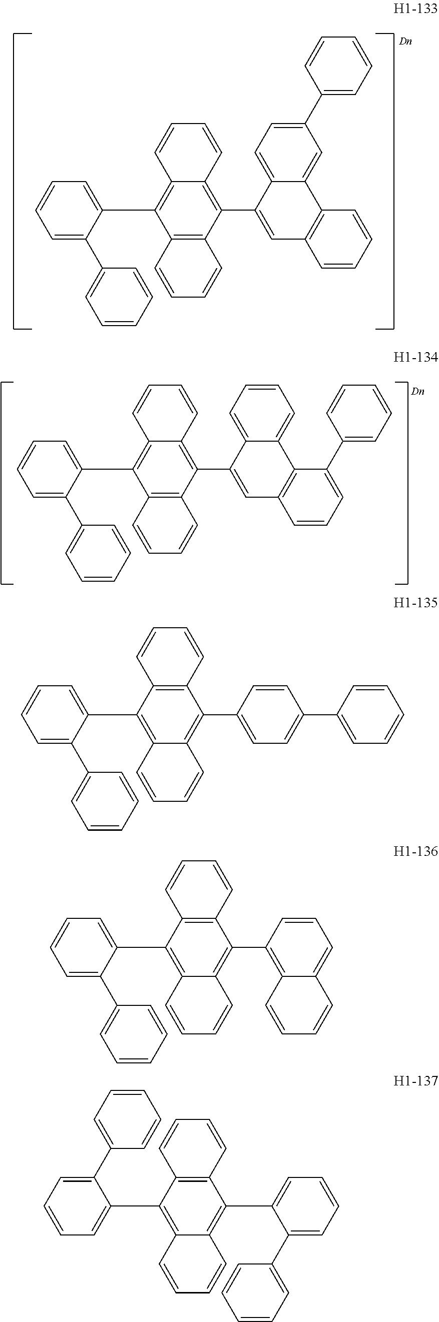 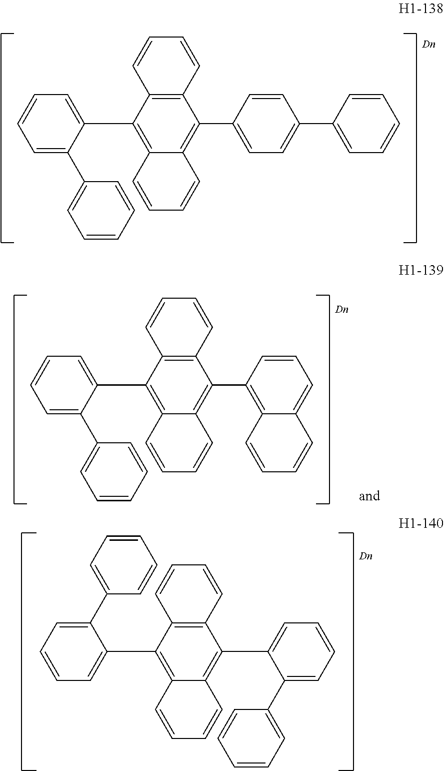
wherein Dₙ signifies that n hydrogens are replaced with deuterium, and n is an integer from 1 to the maximum number of hydrogens in the compound.
